(19) United States
(12) Patent Application Publication
(10) Pub. No.: US 2022/0389031 A1
(43) Pub. Date: Dec. 8, 2022

(54) MACROCYCLIC SULFONYLAMIDE DERIVATIVES USEFUL AS NLRP3 INHIBITORS

(71) Applicant: Inflazome Limited, Dublin 2 (IE)

(72) Inventors: Matthew COOPER, Cambridge (GB); David MILLER, Cambridge (GB); Angus MACLEOD, Cambridge (GB); Stephen THOM, Nottingham (GB); Jonathan SHANNON, Nottingham (GB); Celia Amparo INCERTI-PRADILLOS, Nottingham (GB); Christopher Gordon MCPHERSON, Nottingham (GB)

(21) Appl. No.: 17/635,928

(22) PCT Filed: Aug. 13, 2020
(86) PCT No.: PCT/EP2020/072745

§ 371 Date: Feb. 16, 2022

(30) Foreign Application Priority Data
Aug. 16, 2019 (GB) 1911781.1
Mar. 31, 2020 (GB) 2004682.7
May 22, 2020 (GB) 2007669.1
Jul. 14, 2020 (GB) 2010792.6

(51) Int. Cl. C07D 515/18; C07D 515/08; C07D 515/22; C07D 513/18

(52) U.S. Cl. CPC C07D 515/18; C07D 515/08; C07D 515/22; C07D 513/18

(57) ABSTRACT

The present invention relates to macrocyclic compounds, such as macrocyclic sulfonyl amides. The present invention further relates to associated salts, solvates, prodrugs and pharmaceutical compositions, and to the use of such compounds in the treatment and prevention of medical disorders and diseases, most especially by NLRP₃ inhibition.

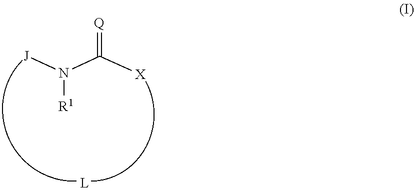

### FIELD OF THE INVENTION

The present invention relates to macrocyclic compounds, such as macrocyclic sulfonyl amides. The present invention further relates to associated salts, solvates, prodrugs and pharmaceutical compositions, and to the use of such compounds in the treatment and prevention of medical disorders and diseases, most especially by NLRP3 inhibition.

### BACKGROUND OF THE INVENTION

The NOD-like receptor (NLR) family, pyrin domain-containing protein 3 (NLRP3) inflammasome is a component of the inflammatory process, and its aberrant activity is pathogenic in inherited disorders such as cryopyrin-associated periodic syndromes (CAPS) and complex diseases such as multiple sclerosis, type 2 diabetes, Alzheimer's disease and atherosclerosis.

NLRP3 is an intracellular signalling molecule that senses many pathogen-derived, environmental and host-derived factors. Upon activation, NLRP3 binds to apoptosis-associated speck-like protein containing a caspase activation and recruitment domain (ASC). ASC then polymerises to form a large aggregate known as an ASC speck. Polymerised ASC in turn interacts with the cysteine protease caspase-1 to form a complex termed the inflammasome. This results in the activation of caspase-1, which cleaves the precursor forms of the proinflammatory cytokines IL-1β and IL-18 (termed pro-IL-1β and pro-IL-18 respectively) to thereby activate these cytokines. Caspase-1 also mediates a type of inflammatory cell death known as pyroptosis. The ASC speck can also recruit and activate caspase-8, which can process pro-IL-1β and pro-IL-18 and trigger apoptotic cell death.

Caspase-1 cleaves pro-IL-1β and pro-IL-18 to their active forms, which are secreted from the cell. Active caspase-1 also cleaves gasdermin-D to trigger pyroptosis. Through its control of the pyroptotic cell death pathway, caspase-1 also mediates the release of alarmin molecules such as IL-33 and high mobility group box 1 protein (HMGB1). Caspase-1 also cleaves intracellular IL-1R2 resulting in its degradation and allowing the release of IL-1α. In human cells caspase-1 may also control the processing and secretion of IL-37. A number of other caspase-1 substrates such as components of the cytoskeleton and glycolysis pathway may contribute to caspase-1-dependent inflammation.

NLRP3-dependent ASC specks are released into the extracellular environment where they can activate caspase-1, induce processing of caspase-1 substrates and propagate inflammation.

Active cytokines derived from NLRP3 inflammasome activation are important drivers of inflammation and interact with other cytokine pathways to shape the immune response to infection and injury. For example, IL-1β signalling induces the secretion of the pro-inflammatory cytokines IL-6 and TNF. IL-1β and IL-18 synergise with IL-23 to induce IL-17 production by memory CD4 Th17 cells and by γδ T cells in the absence of T cell receptor engagement. IL-18 and IL-12 also synergise to induce IFN-γ production from memory T cells and NK cells driving a Th1 response.

The inherited CAPS diseases Muckle-Wells syndrome (MWS), familial cold autoinflammatory syndrome (FCAS) and neonatal-onset multisystem inflammatory disease (NOMID) are caused by gain-of-function mutations in NLRP3, thus defining NLRP3 as a critical component of the inflammatory process. NLRP3 has also been implicated in the pathogenesis of a number of complex diseases, notably including metabolic disorders such as type 2 diabetes, atherosclerosis, obesity and gout.

A role for NLRP3 in diseases of the central nervous system is emerging, and lung diseases have also been shown to be influenced by NLRP3. Furthermore, NLRP3 has a role in the development of liver disease, kidney disease and aging. Many of these associations were defined using Nlrp3−/− mice, but there have also been insights into the specific activation of NLRP3 in these diseases. In type 2 diabetes mellitus (T2D), the deposition of islet amyloid polypeptide in the pancreas activates NLRP3 and IL-1β signalling, resulting in cell death and inflammation.

Several small molecules have been shown to inhibit the NLRP3 inflammasome. Glyburide inhibits IL-1β production at micromolar concentrations in response to the activation of NLRP3 but not NLRC4 or NLRP1. Other previously characterised weak NLRP3 inhibitors include parthenolide, 3,4-methylenedioxy-β-nitrostyrene and dimethyl sulfoxide (DMSO), although these agents have limited potency and are nonspecific.

Current treatments for NLRP3-related diseases include biologic agents that target IL-1. These are the recombinant IL-1 receptor antagonist anakinra, the neutralizing IL-1β antibody canakinumab and the soluble decoy IL-1 receptor rilonacept. These approaches have proven successful in the treatment of CAPS, and these biologic agents have been used in clinical trials for other IL-1β-associated diseases.

Certain sulfonylamide-containing compounds are also disclosed as inhibitors of NLRP3 (see for example WO 2017/184604 A1 and WO 2019/079119 A1), as are certain sulfoximine-containing compounds (see for example WO 2018/225018 A1, WO 2019/023145 A1, WO 2019/023147 A1, and WO 2019/068772 A1).

There is a need to provide compounds with improved pharmacological and/or physiological and/or physicochemical properties and/or those that provide a useful alternative to known compounds.

### SUMMARY OF THE INVENTION

A first aspect of the invention provides a compound of formula (I):

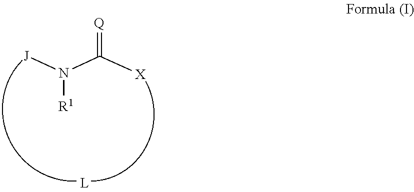

wherein:
J is —SO—, —SO₂— or —SO(═NR^{j})—;
Q is O or S;
X is —C(R²)₂—;
L is a saturated or unsaturated hydrocarbylene group, wherein the hydrocarbylene group may be straight-chained or branched, or be or include one or more cyclic groups, wherein the hydrocarbylene group may optionally be substituted, and wherein the hydrocarbylene group may optionally include one or more heteroatoms independently selected from N, O and S in its carbon skeleton;
J-N(R¹)—C(=Q)-X— and -L- together form a ring, such that the minimum single ring size that encompasses all or part of each of -J-, —N(R¹)—, —C(=Q)-, —X— and -L- is from 8 to 30 atoms;
each R^{j} and R¹ is independently selected from hydrogen or a saturated or unsaturated hydrocarbyl group, wherein the hydrocarbyl group may be straight-chained or branched, or be or include one or more cyclic groups, wherein the hydrocarbyl group may optionally be substituted, and wherein the hydrocarbyl group may optionally include one or more heteroatoms independently selected from N, O and S in its carbon skeleton; and
each R² is independently selected from hydrogen or a halo, —OH, —NO₂, —NH₂, —N₃, —SH, —SO₂H, —SO₂NH₂, or a saturated or unsaturated hydrocarbyl group, wherein the hydrocarbyl group may be straight-chained or branched, or be or include one or more cyclic groups, wherein the hydrocarbyl group may optionally be substituted, and wherein the hydrocarbyl group may optionally include one or more heteroatoms N, O or S in its carbon skeleton, or wherein two R² may, together with the carbon atom to which they are attached, form a cyclic group, wherein the cyclic group may optionally be substituted.

In the context of the present specification, a “hydrocarbyl” substituent group or a hydrocarbyl moiety in a substituent group only includes carbon and hydrogen atoms but, unless stated otherwise, does not include any heteroatoms, such as N, O or S, in its carbon skeleton. A hydrocarbyl group/moiety may be saturated or unsaturated (including aromatic), and may be straight-chained or branched, or be or include cyclic groups wherein, unless stated otherwise, the cyclic group does not include any heteroatoms, such as N, O or S, in its carbon skeleton. Examples of hydrocarbyl groups include alkyl, alkenyl, alkynyl, cycloalkyl, cycloalkenyl and aryl groups/moieties and combinations of all of these groups/moieties. Typically a hydrocarbyl group is a C₁-C₂₀ hydrocarbyl group. More typically a hydrocarbyl group is a C₁-C₁₅ hydrocarbyl group. More typically a hydrocarbyl group is a C₁-C₁₀ hydrocarbyl group. A “hydrocarbylene” group is similarly defined as a divalent hydrocarbyl group.

An “alkyl” substituent group or an alkyl moiety in a substituent group may be linear (i.e. straight-chained) or branched. Examples of alkyl groups/moieties include methyl, ethyl, n-propyl, i-propyl, n-butyl, i-butyl, t-butyl and n-pentyl groups/moieties. Unless stated otherwise, the term “alkyl” does not include “cycloalkyl”. Typically an alkyl group is a C₁-C₁₂ alkyl group. More typically an alkyl group is a C₁-C₆ alkyl group. An “alkylene” group is similarly defined as a divalent alkyl group.

An “alkenyl” substituent group or an alkenyl moiety in a substituent group refers to an unsaturated alkyl group or moiety having one or more carbon-carbon double bonds. Examples of alkenyl groups/moieties include ethenyl, propenyl, 1-butenyl, 2-butenyl, 1-pentenyl, 1-hexenyl, 1,3-butadienyl, 1,3-pentadienyl, 1,4-pentadienyl and 1,4-hexadienyl groups/moieties. Unless stated otherwise, the term “alkenyl” does not include “cycloalkenyl”. Typically an alkenyl group is a C₂-C₁₂ alkenyl group. More typically an alkenyl group is a C₂-C₆ alkenyl group. An “alkenylene” group is similarly defined as a divalent alkenyl group.

An “alkynyl” substituent group or an alkynyl moiety in a substituent group refers to an unsaturated alkyl group or moiety having one or more carbon-carbon triple bonds. Examples of alkynyl groups/moieties include ethynyl, propargyl, but-1-ynyl and but-2-ynyl groups/moieties. Typically an alkynyl group is a C₂-C₁₂ alkynyl group. More typically an alkynyl group is a C₂-C₆ alkynyl group. An “alkynylene” group is similarly defined as a divalent alkynyl group.

A “cyclic” substituent group or a cyclic moiety in a substituent group refers to any hydrocarbyl ring, wherein the hydrocarbyl ring may be saturated or unsaturated (including aromatic) and may include one or more heteroatoms, e.g. N, O or S, in its carbon skeleton. Examples of cyclic groups include cycloalkyl, cycloalkenyl, heterocyclic, aryl and heteroaryl groups as discussed below. A cyclic group may be monocyclic, bicyclic (e.g. bridged, fused or spiro), or polycyclic. Typically, a cyclic group is a 3- to 12-membered cyclic group, which means it contains from 3 to 12 ring atoms. More typically, a cyclic group is a 3- to 7-membered monocyclic group, which means it contains from 3 to 7 ring atoms.

As used herein, where it is stated that a monovalent cyclic group is monocyclic, it is to be understood that the monovalent cyclic group is not substituted with a divalent bridging substituent (e.g. —O—, —S—, —NH—, —N(R^{β})—, —N(O)(R^{β})—, —N⁺(R^{β})₂— or —R^{α}—) so as to form a bridged, fused or spiro substituent. However, unless stated otherwise, a substituted monovalent monocyclic group may be substituted with one or more further monovalent cyclic groups. Similarly, where it is stated that a monovalent cyclic group is bicyclic, it is to be understood that the monovalent cyclic group including any bridged, fused or spiro divalent bridging substituents attached to the monovalent cyclic group, but excluding any monovalent cyclic substituents, is bicyclic.

Likewise, where it is stated that a divalent cyclic group is monocyclic, it is to be understood that while one or more bridged, fused or spiro ring structures may be formed via the two positions of attachment of the divalent cyclic group to the remainder of the molecule, the divalent cyclic group is not substituted at other positions with a divalent bridging substituent (e.g. —O—, —S—, —NH—, —N(R^{β})—, —N(O)(R^{β})—, —N⁺(R^{β})₂— or —R^{α}—) so as to form a further bridged, fused or spiro substituent. However, unless stated otherwise, a substituted divalent monocyclic group may be substituted with one or more further monovalent cyclic groups. Similarly, where it is stated that a divalent cyclic group is bicyclic, it is to be understood that the divalent cyclic group including any bridged, fused or spiro divalent bridging substituents attached to the cyclic group, but excluding any monovalent cyclic substituents or any structures formed via the two positons of attachment of the divalent cyclic group to the remainder of the molecule, is bicyclic.

A “heterocyclic” substituent group or a heterocyclic moiety in a substituent group refers to a cyclic group or moiety including one or more carbon atoms and one or more (such as one, two, three or four) heteroatoms, e.g. N, O or S, in the ring structure. Examples of heterocyclic groups include heteroaryl groups as discussed below and non-aromatic heterocyclic groups such as azetinyl, azetidinyl, oxetanyl, thietanyl, pyrrolidinyl, tetrahydrofuranyl, tetrahydrothiophenyl, pyrazolidinyl, imidazolidinyl, dioxolanyl, oxathiolanyl, piperidinyl, tetrahydropyranyl, thianyl, piperazinyl, dioxanyl, morpholinyl and thiomorpholinyl groups.

A “cycloalkyl” substituent group or a cycloalkyl moiety in a substituent group refers to a saturated hydrocarbyl ring containing, for example, from 3 to 7 carbon atoms, examples of which include cyclopropyl, cyclobutyl, cyclopentyl and cyclohexyl. Unless stated otherwise, a cycloalkyl substituent group or moiety may include monocyclic, bicyclic or polycyclic hydrocarbyl rings.

A “cycloalkenyl” substituent group or a cycloalkenyl moiety in a substituent group refers to a non-aromatic unsaturated hydrocarbyl ring having one or more carbon-carbon double bonds and containing, for example, from 3 to 7 carbon atoms, examples of which include cyclopent-1-en-1-yl, cyclohex-1-en-1-yl and cyclohex-1,3-dien-1-yl.

Unless stated otherwise, a cycloalkenyl substituent group or moiety may include monocyclic, bicyclic or polycyclic hydrocarbyl rings.

An “aryl” substituent group or an aryl moiety in a substituent group refers to an aromatic hydrocarbyl ring. The term “aryl” includes monocyclic aromatic hydrocarbons and polycyclic fused ring aromatic hydrocarbons wherein all of the fused ring systems (excluding any ring systems which are part of or formed by optional substituents) are aromatic. Examples of aryl groups/moieties include phenyl, naphthyl, anthracenyl and phenanthrenyl. Unless stated otherwise, the term “aryl” does not include “heteroaryl”.

A “heteroaryl” substituent group or a heteroaryl moiety in a substituent group refers to an aromatic heterocyclic group or moiety. The term “heteroaryl” includes monocyclic aromatic heterocycles and polycyclic fused ring aromatic heterocycles wherein all of the fused ring systems (excluding any ring systems which are part of or formed by optional substituents) are aromatic. Examples of heteroaryl groups/moieties include the following:

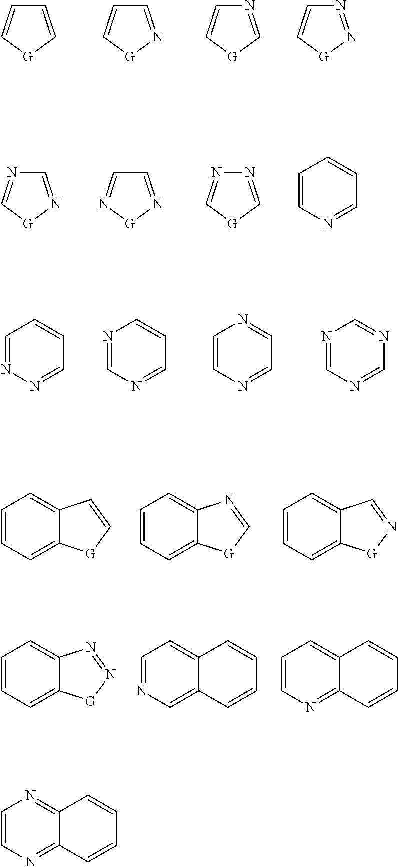

wherein G=O, S or NH. Particular examples of 5- or 6-membered heteroaryl groups include furanyl, thiophenyl, pyrrolyl, imidazolyl, pyrazolyl, oxazolyl, isoxazolyl, thiazolyl, isothiazolyl, triazolyl, furazanyl, oxadiazolyl, thiadiazolyl, tetrazolyl, pyridinyl, pyridazinyl, pyrimidinyl, pyrazinyl and triazinyl groups.

Unless stated otherwise, where a cyclic group or moiety is stated to be non-aromatic, such as a cycloalkyl, cycloalkenyl or non-aromatic heterocyclic group, it is to be understood that the group or moiety, excluding any ring systems which are part of or formed by substituents, is non-aromatic. Similarly, where a cyclic group or moiety is stated to be aromatic, such as an aryl or a heteroaryl group, it is to be understood that the group or moiety, excluding any ring systems which are part of or formed by substituents, is aromatic. A cyclic group or moiety is considered non-aromatic, when it does not have any tautomers that are aromatic. When a cyclic group or moiety has a tautomer that is aromatic, it is considered aromatic, even if it has tautomers that are not aromatic. Byway of example, the following are considered aromatic heterocyclic groups, because they have an aromatic tautomer:

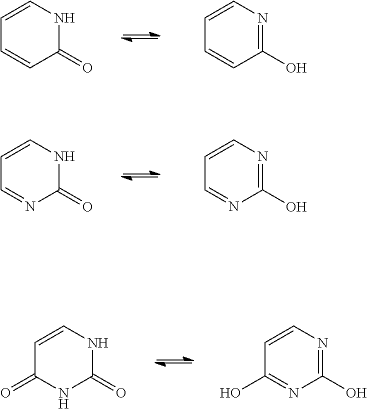 [SMILES: O=c1cccc[nH]1 ; Oc1ccccn1 ; O=c1nccc[nH]1 ; Oc1ncccn1 ; O=c1cc[nH]c(=O)[nH]1 ; Oc1ccnc(O)n1]

For the avoidance of doubt, the term “non-aromatic heterocyclic group” does not exclude heterocyclic groups or moieties which may possess aromatic character only by virtue of mesomeric charge separation. For example, the following is considered a non-aromatic heterocyclic group, because it does not have an aromatic tautomer:

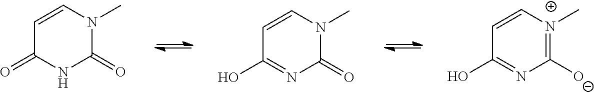

because the last shown structure is not taken into consideration because of mesomeric charge separation.

For the avoidance of doubt, where it is stated that a bicyclic or polycyclic group is “saturated” it is to be understood that all of the ring systems within the bicyclic or polycyclic group (excluding any ring systems which are part of or formed by optional substituents) are saturated.

For the purposes of the present specification, where a combination of moieties is referred to as one group, for example, arylalkyl, arylalkenyl, arylalkynyl, alkylaryl, alkenylaryl or alkynylaryl, the last mentioned moiety contains the atom by which the group is attached to the rest of the molecule. An example of an arylalkyl group is benzyl.

For the purposes of the present specification, in an optionally substituted group or moiety, such as L:

(i) each hydrogen atom may optionally be replaced by a monovalent substituent independently selected from halo; —CN; —NO₂; —N₃; —R^{β}; —OH; —OR^{β}; —R^{α}-halo; —R^{α}—CN; —R^{α}—NO₂; —R^{α}—N₃; —R^{α}—R^{β}; —R^{α}—OH; —R^{α}—OR^{β}; —SH; —SR^{β}; —SOR^{β}; —SO₂H; —SO₂R^{β}; —SO₂NH₂; —SO₂NHR^{β}; —SO₂N(R^{β})₂; —R^{α}—SH; —R^{α}—SR^{β}; —R^{α}—SOR^{β}; —R^{α}—SO₂H; —R^{α}—SO₂R^{β}; —R^{α}—SO₂NH₂; —R^{α}—SO₂NHR^{β}; —R^{α}—SO₂N(R^{β})₂; —Si(R^{β})₃; —O—Si(R^{β})₃; —R^{α}—Si(R^{β})₃; —R^{α}—O—Si(R^{β})₃; —NH₂; —NHR^{β}; —N(R^{β})₂; —N(O)(R^{β})₂; —N⁺(R^{β})₃; —R^{α}—NH₂; —R^{α}—NHR^{β}; —R^{α}—N(R^{β})₂; —R^{α}—N(O)(R^{β})₂; —R^{α}—N⁺(R^{β})₃; —CHO; —COR^{β}; —COOH; —COOR^{β}; —OCOR^{β}; —R^{α}—CHO; —R^{α}—COR^{β}; —R^{α}—COOH; —R^{α}—COOR^{β}; —R^{α}—OCOR^{β}; —C(═NH)R^{β}; —C(═NH)NH₂; —C(═NH)NHR^{β}; —C(═NH)N(R^{β})₂; —C(═NR^{β})R^{β}; —C(═NR^{β})NHR^{β}; —C(═NR^{β})N(R^{β})₂; —C(═NOH)R^{β}; —C(═NOR^{β})R^{β}; —C(N₂)R^{β}; —R^{α}—C(═NH)R^{β}; —R^{α}—C(═NH)NH₂; —R^{α}—C(═NH)NHR^{β}; —R^{α}—C(═NH)N(R^{β})₂; —R^{α}—C(═NR^{β})R^{β}; —R^{α}—C(═NR^{β})NHR^{β}; —R^{α}—C(═NR^{β})N(R^{β})₂; —R^{α}—C(═NOH)R^{β}; —R^{α}—C(═NOR^{β})R^{β}; —R^{α}—C(N₂)R^{β}; —NH—CHO; —NR^{β}—CHO; —NH—COR^{β}; —NR^{β}—COR^{β}; —NH—COOR^{β}; —NR^{β}—COOR^{β}; —NH—C(═NH)R^{β}; —NR^{β}—C(═NH)R^{β}; —NH—C(═NH)NH₂; —NR^{β}—C(═NH)NH₂; —NH—C(═NH)NHR^{β}; —NR^{β}—C(═NH)NHR^{β}; —NH—C(═NH)N(R^{β})₂; —NR^{β}—C(═NH)N(R^{β})₂; —NH—C(═NR^{β})R^{β}; —NR^{β}—C(═NR^{β})R^{β}; —NH—C(═NR^{β})NHR^{β}; —NR^{β}—C(═NR^{β})NHR^{β}; —NH—C(═NR^{β})N(R^{β})₂; —NR^{β}—C(═NR^{β})N(R^{β})₂; —NH—C(═NOH)R^{β}; —NR^{β}—C(═NOH)R^{β}; —NH—C(═NOR^{β})R^{β}; —NR^{β}—C(═NOR^{β})R^{β}; —CONH₂; —CONHR^{β}; —CON(R^{β})₂; —NH—CONH₂; —NR^{β}—CONH₂; —NH—CONHR^{β}; —NR^{β}—CONHR^{β}; —NH—CON(R^{β})₂; —NR^{β}—CON(R^{β})₂; —R^{α}—NH—CHO; —R^{α}—NR^{β}—CHO; —R^{α}—NH—COR^{β}; —R^{α}—NR^{β}—COR^{β}; —R^{α}—NH—COOR^{β}; —R^{α}—NR^{β}—COOR^{β}; —R^{α}—NH—C(═NH)R^{β}; —R^{α}—NR^{β}—C(═NH)R^{β}; —R^{α}—NH—C(═NH)NH₂; —R^{α}—NR^{β}—C(═NH)NH₂; —R^{α}—NH—C(═NH)NHR^{β}; —R^{α}—NR^{β}—C(═NH)NHR^{β}; —R^{α}—NH—C(═NH)N(R^{β})₂; —R^{α}—NR^{β}—C(═NH)N(R^{β})₂; —R^{α}—NH—C(═NR^{β})R^{β}; —R^{α}—NR^{β}—C(═NR^{β})R^{β}; —R^{α}—NH—C(═NR^{β})NHR^{β}; —R^{α}—NR^{β}—C(═NR^{β})NHR^{β}; —R^{α}—NH—C(═NR^{β})N(R^{β})₂; —R^{α}—NR^{β}—C(═NR^{β})N(R^{β})₂; —R^{α}—NH—C(═NOH)R^{β}; —R^{α}—NR^{β}—C(═NOH)R^{β}; —R^{α}—NH—C(═NOR^{β})R^{β}; —R^{α}—NR^{β}—C(═NOR^{β})R^{β}; —R^{α}—CONH₂; —R^{α}—CONHR^{β}; —R^{α}—CON(R^{β})₂; —R^{α}—NH—CONH₂; —R^{α}—NR^{β}—CONH₂; —R^{α}—NH—CONHR^{β}; —R^{α}—NR^{β}—CONHR^{β}; —R^{α}—NH—CON(R^{β})₂; —R^{α}—NR^{β}—CON(R^{β})₂; —O—R^{α}—OH; —O—R^{α}—OR^{β}; —O—R^{α}—NH₂; —O—R^{α}—NHR^{β}; —O—R^{α}—N(R^{β})₂; —O—R^{α}—N(O)(R^{β})₂; —O—R^{α}—N⁺(R^{β})₃; —NH—R^{α}—OH; —NH—R^{α}—OR^{β}; —NH—R^{α}—NH₂; —NH—R^{α}—NHR^{β}; —NH—R^{α}—N(R^{β})₂; —NH—R^{α}—N(O)(R^{β})₂; —NH—R^{α}—N⁺(R^{β})₃; —NR^{β}—R^{α}—OH; —NR^{β}—R^{α}—OR^{β}; —NR^{β}—R^{α}—NH₂; —NR^{β}—R^{α}—NHR^{β}; —NR^{β}—R^{α}—N(R^{β})₂; —NR^{β}—R^{α}—N(O)(R^{β})₂; —NR^{β}—R^{α}—N⁺(R^{β})₃; —N(O)R^{β}—R^{α}—OH; —N(O)R^{β}—R^{α}—OR^{β}; —N(O)R^{β}—R^{α}—NH₂; —N(O)R^{β}—R^{α}—NHR^{β}; —N(O)R^{β}—R^{α}—N(R^{β})₂; —N(O)R^{β}—R^{α}—N(O)(R^{β})₂; —N(O)R^{β}—R^{α}—N⁺(R^{β})₃; —N⁺(R^{β})₂—R^{α}—OH; —N⁺(R^{β})₂—R^{α}—OR^{β}; —N⁺(R^{β})₂—R^{α}—NH₂; —N⁺(R^{β})₂—R^{α}—NHR^{β}; —N⁺(R^{β})₂—R^{α}—N(R^{β})₂; or —N⁺(R^{β})₂—R^{α}—N(O)(R^{β})₂; and/or

(ii) any two hydrogen atoms attached to the same carbon or nitrogen atom may optionally be replaced by a π-bonded substituent independently selected from oxo (═O), ═S, ═NH or ═NR^{β}; and/or

(iii) any sulfur atom may optionally be substituted with one or two π-bonded substituents independently selected from oxo (═O), =NH or ═NR^{β}; and/or

(iv) any two hydrogen atoms attached to the same or different atoms, within the same optionally substituted group or moiety, may optionally be replaced by a bridging substituent independently selected from —O—, —S—, —NH—, —N═N—, —N(R^{β})—, —N(O)(R^{β})—, —N⁺(R^{β})₂— or —R^{α}—;
wherein each —R^{α}— is independently selected from an alkylene, alkenylene or alkynylene group, wherein the alkylene, alkenylene or alkynylene group contains from 1 to 6 atoms in its backbone, wherein one or more carbon atoms in the backbone of the alkylene, alkenylene or alkynylene group may optionally be replaced by one or more heteroatoms N, O or S, wherein one or more —CH₂— groups in the backbone of the alkylene, alkenylene or alkynylene group may optionally be replaced by one or more —N(O)(R^{β})— or —N⁺(R^{β})₂— groups, and wherein the alkylene, alkenylene or alkynylene group may optionally be substituted with one or more halo and/or —R³ groups; and
wherein each —RR is independently selected from a C₁-C₆ alkyl, C₂-C₆ alkenyl, C₂-C₆ alkynyl or C₂-C₆ cyclic group, or wherein any two or three —R^{β} attached to the same nitrogen atom may, together with the nitrogen atom to which they are attached, form a C₂-C₇ cyclic group, and wherein any —R³ may optionally be substituted with one or more C₁-C₄ alkyl, C₁-C₄ haloalkyl, C₃-C₇ cycloalkyl, C₃-C₇ halocycloalkyl, —O(C₁-C₄ alkyl), —O(C₁-C₄ haloalkyl), —O(C₃-C₇ cycloalkyl), —O(C₃-C₇ halocycloalkyl), —CO(C₁-C₄ alkyl), —CO(C₁-C₄ haloalkyl), —CO(C₃-C₇ cycloalkyl), —CO(C₃-C₇ halocycloalkyl), —COO(C₁-C₄ alkyl), —COO(C₁-C₄ haloalkyl), —COO(C₃-C₇ cycloalkyl), —COO(C₃-C₇ halocycloalkyl), halo, —OH, —NH₂, —CN, —C—CH, oxo (═O), phenyl, halophenyl, or optionally halo-substituted 4- to 6-membered heterocyclic group.

Typically, the compounds of the present invention comprise at most one quaternary ammonium group such as —N⁺(R^{β})₃ or —N⁺(R^{β})₂—.

Where reference is made to a —R^{α}—C(N₂)R^{β} group, what is intended is:

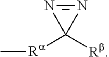

Typically a substituted group comprises 1, 2, 3 or 4 substituents, more typically 1, 2 or 3 substituents, more typically 1 or 2 substituents, and more typically 1 substituent.

Unless stated otherwise, any optional substituent is only attached to the group or moiety which is optionally substituted. For example, any divalent bridging substituent (e.g. —O—, —S—, —NH—, —N(R^{β})—, —N(O)(R^{β})—, —N⁺(R^{β})₂— or —R^{α}—) of an optionally substituted group or moiety (e.g. R¹) must only be attached to the specified group or moiety and may not be attached to a second group or moiety (e.g. R²), even if the second group or moiety can itself be optionally substituted.

The term “halo” includes fluoro, chloro, bromo and iodo.

Unless stated otherwise, where a group is prefixed by the term “halo”, such as a haloalkyl or halomethyl group, it is to be understood that the group in question is substituted with one or more halo groups independently selected from fluoro, chloro, bromo and iodo. Typically, the maximum number of halo substituents is limited only by the number of hydrogen atoms available for substitution on the corresponding group without the halo prefix. For example, a halomethyl group may contain one, two or three halo substituents. A haloethyl or halophenyl group may contain one, two, three, four or five halo substituents. Similarly, unless stated otherwise, where a group is prefixed by a specific halo group, it is to be understood that the group in question is substituted with one or more of the specific halo groups. For example, the term “fluoromethyl” refers to a methyl group substituted with one, two or three fluoro groups.

Similarly, unless stated otherwise, where a group is said to be “halo-substituted”, it is to be understood that the group in question is substituted with one or more halo groups independently selected from fluoro, chloro, bromo and iodo. Typically, the maximum number of halo substituents is limited only by the number of hydrogen atoms available for substitution on the group said to be halo-substituted. For example, a halo-substituted methyl group may contain one, two or three halo substituents. A halo-substituted ethyl or halo-substituted phenyl group may contain one, two, three, four or five halo substituents.

Unless stated otherwise, any reference to an element is to be considered a reference to all isotopes of that element. Thus, for example, unless stated otherwise any reference to hydrogen is considered to encompass all isotopes of hydrogen including deuterium and tritium.

Unless stated otherwise, any reference to a compound or group is to be considered a reference to all tautomers of that compound or group.

Where reference is made to a hydrocarbyl or other group including one or more heteroatoms N, O or S in its carbon skeleton, or where reference is made to a carbon atom of a hydrocarbyl or other group being replaced by an N, O or S atom, what is intended is that:

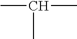

is replaced by

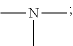

—CH₂— is replaced by —NH—, —O— or —S—;
—CH₃ is replaced by —NH₂, —OH or —SH;
—CH═ is replaced by —N═;
CH₂═ is replaced by NH═, O═ or S═; or
CH≡ is replaced by N≡;

provided that the resultant group comprises at least one carbon atom. For example, methoxy, dimethylamino and aminoethyl groups are considered to be hydrocarbyl groups including one or more heteroatoms N, O or S in their carbon skeleton.

Where reference is made to a —CH₂— group in the backbone of a hydrocarbyl or other group being replaced by a —N(O)(R^{β})— or —N⁺(R^{β})₂— group, what is intended is that:
—CH₂— is replaced by

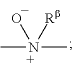

—CH₂— is replaced by

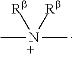

In the context of the present specification, unless otherwise stated, a Cₓ-C_{y} group is defined as a group containing from x to y carbon atoms. For example, a C₁-C₄ alkyl group is defined as an alkyl group containing from 1 to 4 carbon atoms. Optional substituents and moieties are not taken into account when calculating the total number of carbon atoms in the parent group substituted with the optional substituents and/or containing the optional moieties. For the avoidance of doubt, replacement heteroatoms, e.g. N, O or S, are not to be counted as carbon atoms when calculating the number of carbon atoms in a Cₓ-C_{y} group. For example, a morpholinyl group is to be considered a C₄ heterocyclic group, not a C₆ heterocyclic group.

For the purposes of the present specification, where it is stated that a first atom or group is “directly attached” to a second atom or group it is to be understood that the first atom or group is covalently bonded to the second atom or group with no intervening atom(s) or group(s) being present. So, for example, for the group —(C═O)N(CH₃)₂, the carbon atom of each methyl group is directly attached to the nitrogen atom and the carbon atom of the carbonyl group is directly attached to the nitrogen atom, but the carbon atom of the carbonyl group is not directly attached to the carbon atom of either methyl group.

For the avoidance of doubt, where it is stated that a compound or a group, such as R¹, R² or L, contains from x to y atoms other than hydrogen or halogen, it is to be understood that the compound or group as a whole, including any optional substituents, contains from x to y atoms other than hydrogen or halogen. Such a compound or group may contain any number of hydrogen or halogen atoms. Similarly, where it is stated that a compound or a group, such as R¹, R² or L, contains from x to y atoms other than hydrogen, it is to be understood that the compound or group as a whole, including any optional substituents, contains from x to y atoms other than hydrogen. Such a compound or group may contain any number of hydrogen atoms.

As stated, J is —SO—, —SO₂— or —SO(═NR^{j})—. More typically, J is —SO₂— or —SO(═NR^{j})—.

As stated, R^{j} is selected from hydrogen or a saturated or unsaturated hydrocarbyl group, wherein the hydrocarbyl group may be straight-chained or branched, or be or include one or more cyclic groups, wherein the hydrocarbyl group may optionally be substituted, and wherein the hydrocarbyl group may optionally include one or more heteroatoms independently selected from N, O and S in its carbon skeleton.

In one embodiment, R^{j} is selected from hydrogen, —CN or a saturated C₁-C₆ hydrocarbyl group, wherein the saturated C₁-C₆ hydrocarbyl group may be straight-chained or branched, or be or include a cyclic group, wherein the saturated C₁-C₆ hydrocarbyl group may optionally include one or two heteroatoms independently selected from N and O in its carbon skeleton, and wherein the saturated C₁-C₆ hydrocarbyl group may optionally be substituted with one or more groups independently selected from halo, —CN, —OH, —NH₂ and oxo (═O).

More typically, R^{j} is selected from hydrogen, —CN or a C₁-C₄ alkyl, C₁-C₄ fluoroalkyl, C₃-C₄ cycloalkyl or C₃-C₄ fluorocycloalkyl group. For example, R^{j} may be selected from hydrogen, —CN, or a methyl, ethyl, n-propyl, isopropyl or cyclopropyl group, wherein any methyl, ethyl, n-propyl, isopropyl or cyclopropyl group may optionally be substituted with one or more fluoro groups.

Yet more typically, R^{j} is selected from hydrogen or —CN. Most typically, R^{j} is hydrogen.

In one embodiment, J is —SO—, —SO₂— or —SO(═NH)—. More typically in such an embodiment, J is —SO₂— or —SO(═NH)—.

Most typically, J is —SO₂—.

As stated, Q is O or S. Most typically, Q is O.

As stated, R¹ is selected from hydrogen or a saturated or unsaturated hydrocarbyl group, wherein the hydrocarbyl group may be straight-chained or branched, or be or include one or more cyclic groups, wherein the hydrocarbyl group may optionally be substituted, and wherein the hydrocarbyl group may optionally include one or more heteroatoms independently selected from N, O and S in its carbon skeleton.

In one embodiment, R¹ is selected from hydrogen or a saturated C₁-C₆ hydrocarbyl group, wherein the saturated C₁-C₆ hydrocarbyl group may be straight-chained or branched, or be or include a cyclic group, wherein the saturated C₁-C₆ hydrocarbyl group may optionally include one or two heteroatoms independently selected from N and O in its carbon skeleton, and wherein the saturated C₁-C₆ hydrocarbyl group may optionally be substituted with one or more groups independently selected from halo, —CN, —OH, —NH₂ and oxo (═O).

More typically, R¹ is selected from hydrogen or a C₁-C₄ alkyl, C₁-C₄ fluoroalkyl, C₃-C₄ cycloalkyl or C₃-C₄ fluorocycloalkyl group. For example, R¹ may be selected from hydrogen or a methyl, ethyl, n-propyl, isopropyl or cyclopropyl group, wherein any methyl, ethyl, n-propyl, isopropyl or cyclopropyl group may optionally be substituted with one or more fluoro groups.

Yet more typically, R¹ is selected from hydrogen or a methyl group, wherein the methyl group may optionally be substituted with one or more fluoro groups. More typically still, R¹ is hydrogen.

As stated, each R² is independently selected from hydrogen or a halo, —OH, —NO₂, —NH₂, —N₃, —SH, —SO₂H, —SO₂NH₂, or a saturated or unsaturated hydrocarbyl group, wherein the hydrocarbyl group may be straight-chained or branched, or be or include one or more cyclic groups, wherein the hydrocarbyl group may optionally be substituted, and wherein the hydrocarbyl group may optionally include one or more heteroatoms N, O or S in its carbon skeleton, or wherein two R² may, together with the carbon atom to which they are attached, form a cyclic group, wherein the cyclic group may optionally be substituted.

In one embodiment, each R² is independently selected from hydrogen or a halo, —OH, —NO₂, —NH₂, —N₃, —SH, —SO₂H, —SO₂NH₂, or a saturated or unsaturated hydrocarbyl group, wherein the hydrocarbyl group may be straight-chained or branched, or be or include one or more cyclic groups, wherein the hydrocarbyl group may optionally be substituted, and wherein the hydrocarbyl group may optionally include one or more heteroatoms N, O or S in its carbon skeleton. Typically in such an embodiment, each R² is independently selected from hydrogen or a halo, —CN, —OH, —NH₂, or a saturated C₁-C₆ hydrocarbyl group, wherein the saturated C₁-C₆ hydrocarbyl group may be straight-chained or branched, or be or include a cyclic group, wherein the saturated C₁-C₆ hydrocarbyl group may optionally include one or two heteroatoms independently selected from N and O in its carbon skeleton, and wherein the saturated C₁-C₆ hydrocarbyl group may optionally be substituted with one or more groups independently selected from halo, —CN, —OH, —NH₂ and oxo (═O).

In another embodiment, two R², together with the carbon atom to which they are attached, form a cyclic group, wherein the cyclic group may optionally be substituted. As will be understood, such a cyclic group is attached to the ring formed by -J-N(R¹)—C(=Q)-X- and -L- as a spiro-group. Typically in such an embodiment, two R², together with the carbon atom to which they are attached, together form a 3- to 7-membered saturated cyclic group, wherein the saturated cyclic group may optionally include one or two ring heteroatoms independently selected from N and O in its carbon skeleton, and wherein the saturated cyclic group may optionally be substituted with one or more groups independently selected from halo, —CN, —OH, —NH₂ and oxo (═O).

More typically, each R² is independently selected from hydrogen or a fluoro or a C₁-C₄ alkyl, C₁-C₄ fluoroalkyl, C₃-C₄ cycloalkyl or C₃-C₄ fluorocycloalkyl group, or two R² may, together with the carbon atom to which they are attached, form a 3- or 4-membered cycloalkyl group, or form an oxetanyl group, wherein the 3- or 4-membered cycloalkyl group or the oxetanyl group may optionally be fluoro-substituted. For example, each R² may independently be selected from hydrogen or a fluoro or a methyl, ethyl, n-propyl, isopropyl or cyclopropyl group, or two R² may, together with the carbon atom to which they are attached, form a cyclopropyl group, wherein any methyl, ethyl, n-propyl, isopropyl or cyclopropyl group may optionally be substituted with one or more fluoro groups.

Yet more typically, each R² is independently selected from hydrogen or a fluoro or a methyl group, wherein the methyl group may optionally be substituted with one or more fluoro groups. More typically still, each R² is hydrogen, i.e. X is —CH₂—.

Most typically, in accordance with any of the above embodiments, R¹ is hydrogen and X is —CH₂—.

As stated, L is a saturated or unsaturated hydrocarbylene group, wherein the hydrocarbylene group may be straight-chained or branched, or be or include one or more cyclic groups, wherein the hydrocarbylene group may optionally be substituted, and wherein the hydrocarbylene group may optionally include one or more heteroatoms independently selected from N, O and S in its carbon skeleton. Typically, the atom of the hydrocarbylene group that is directly attached to X is a carbon or a nitrogen atom. Typically, the atom of the hydrocarbylene group that is directly attached to J is a carbon or a nitrogen atom.

In one embodiment, L is a saturated or unsaturated hydrocarbylene group, wherein the hydrocarbylene group may be straight-chained or branched, or be or include one or more cyclic groups, wherein the hydrocarbylene group may optionally be substituted, and wherein the hydrocarbylene group may optionally include one or more heteroatoms independently selected from N and O in its carbon skeleton.

Typically the hydrocarbylene group of L includes at least one cyclic group. For example, L may be a saturated or unsaturated hydrocarbylene group, wherein the hydrocarbylene group may be straight-chained or branched, wherein the hydrocarbylene group includes a cyclic group directly attached to X, wherein the hydrocarbylene group may optionally include one or more further cyclic groups, wherein the hydrocarbylene group may optionally be substituted, and wherein the hydrocarbylene group may optionally include one or more heteroatoms independently selected from N, O and S in its carbon skeleton. Typically in such an embodiment, the cyclic group directly attached to X is aromatic.

Typically L, including any optional substituents, contains in total from 1 to 10 nitrogen, oxygen and sulfur atoms. More typically L, including any optional substituents, contains in total from 2 to 8 nitrogen, oxygen and sulfur atoms. Yet more typically L, including any optional substituents, contains in total from 2 to 6 nitrogen, oxygen and sulfur atoms.

In one embodiment, L contains only atoms selected from the group consisting of hydrogen, halo, carbon, nitrogen and oxygen atoms. Typically in such an embodiment L, including any optional substituents, contains in total from 1 to 10 nitrogen and oxygen atoms. More typically L, including any optional substituents, contains in total from 2 to 8 nitrogen and oxygen atoms. Yet more typically L, including any optional substituents, contains in total from 2 to 6 nitrogen and oxygen atoms.

Typically L, including any optional substituents, contains in total from 10 to 40 carbon atoms. More typically L, including any optional substituents, contains in total from 15 to 30 carbon atoms.

Typically L, including any optional substituents, contains in total from 4 to 50 carbon, nitrogen, oxygen and sulfur atoms. More typically L, including any optional substituents, contains in total from 10 to 40 carbon, nitrogen, oxygen and sulfur atoms. More typically still L, including any optional substituents, contains in total from 20 to 35 carbon, nitrogen, oxygen and sulfur atoms.

As stated, -J-N(R¹)—C(=Q)-X- and -L- together form a ring, such that the minimum single ring size that encompasses all or part of each of -J-, —N(R¹)—, —C(=Q)-, —X— and -L- is from 8 to 30 atoms. Typically, the minimum single ring size that encompasses all or part of each of -J-, —N(R¹)—, —C(=Q)-, —X— and -L- is from 12 to 24 atoms. More typically, the minimum single ring size that encompasses all or part of each of -J-, —N(R¹)—, —C(=Q)-, —X— and -L- is from 14 to 20 atoms.

As will be understood, the compounds of the invention may be monocyclic ring systems, or may be bicyclic, tricyclic or polycyclic ring systems, for example due to the presence of cyclic groups within -L-. However, the compounds of formula (I) must meet the criteria that -J-N(R¹)—C(=Q)-X- and -L- together form a ring, such that the minimum single ring size that encompasses all or part of each of -J-, —N(R¹)—, —C(=Q)-, —X— and -L- is from 8 to 30 atoms. It will be appreciated that for bicyclic, tricyclic or polycyclic ring systems, alternate single ring sizes that encompass all or part of each of -J-, —N(R¹)—, —C(=Q)-, —X— and -L- may be identified; it is the smallest of these possible alternate single ring sizes that is relevant for determining the minimum ring size. By way of example, consider the bicyclic structure (A) below:

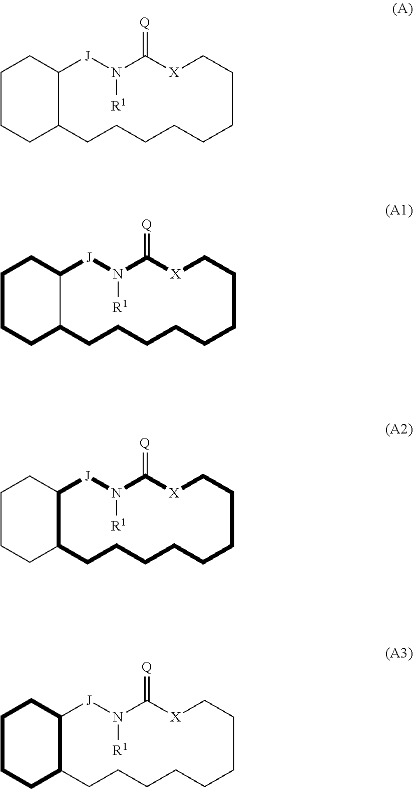

Three single ring sizes within the bicyclic structure may be identified, namely a 18-atom ring illustrated in bold in structure (A1), a 14-atom ring illustrated in bold in structure (A2), and a 6-atom ring illustrated in bold in structure (A3). Of these three single ring sizes, only the two rings illustrated in bold in (A1) and (A2) encompass all or part of each of -J-, —N(R¹)—, —C(=Q)-, —X— and -L-. Of these two rings, the ring illustrated in bold in structure (A2) is the smallest. Hence for structure (A), the minimum single ring size that encompasses all or part of each of -J-, —N(R¹)—, —C(=Q)-, —X— and -L- is 14 atoms. In one embodiment of the first aspect of the invention, the compound has the formula (Ia):

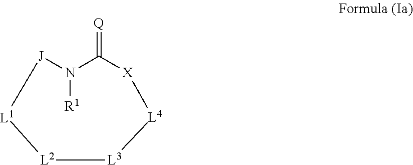

wherein:
J, R¹, Q and X are as previously defined;
J-N(R¹)—C(=Q)-X- and -L¹-L²-L³-L⁴- together form a ring, such that the minimum single ring size that encompasses all or part of each of -J-, —N(R¹)—, —C(=Q)-, —X—, -L¹-, -L²-, -L³- and -L⁴- is from 8 to 30 atoms;
L¹ is a bond, a divalent 3- to 7-membered monocyclic group, a divalent 5- to 12-membered bicyclic group, or a divalent 7- to 18-membered tricyclic group, any of which may optionally be substituted with one or more monovalent substituents and/or π-bonded substituents;
L² is an alkylene, alkenylene or alkynylene group, wherein the alkylene, alkenylene or alkynylene group may be straight-chained or branched, or be or include one or more cyclic groups, wherein one or more carbon atoms in the backbone of the alkylene, alkenylene or alkynylene group may optionally be replaced by one or more heteroatoms independently selected from N, O and S, and wherein the alkylene, alkenylene or alkynylene group may optionally be substituted with one or more monovalent substituents, and/or one or more π-bonded substituents;
L³ is a bond, a divalent 3- to 7-membered monocyclic group, a divalent 5- to 12-membered bicyclic group, or a divalent 7- to 18-membered tricyclic group, any of which may optionally be substituted with one or more monovalent substituents and/or π-bonded substituents; and
L⁴ is a divalent 3- to 7-membered monocyclic group, a divalent 5- to 12-membered bicyclic group, or a divalent 7- to 18-membered tricyclic group, any of which may optionally be substituted with one or more monovalent substituents and/or π-bonded substituents.

In one aspect of such an embodiment, where the compound has the formula (Ia):
L¹ is a bond, a divalent 3- to 7-membered monocyclic group, a divalent 5- to 11-membered bicyclic group, or a divalent 7- to 16-membered tricyclic group, any of which may optionally be substituted with one or more monovalent substituents and/or π-bonded substituents;
L³ is a bond, a divalent 3- to 7-membered monocyclic group, a divalent 5- to 11-membered bicyclic group, or a divalent 7- to 16-membered tricyclic group, any of which may optionally be substituted with one or more monovalent substituents and/or π-bonded substituents; and
L⁴ is a divalent 3- to 7-membered monocyclic group, a divalent 5- to 11-membered bicyclic group, or a divalent 7- to 16-membered tricyclic group, any of which may optionally be substituted with one or more monovalent substituents and/or π-bonded substituents.

For the avoidance of doubt:
where L¹ is a divalent 3- to 7-membered monocyclic group, a divalent 5- to 11- or 5- to 12-membered bicyclic group, or a divalent 7- to 16- or 7- to 18-membered tricyclic group, a ring atom of the monocyclic, bicyclic or tricyclic group of L¹ is directly attached to the sulfur atom of J, and the same or a different ring atom of the monocyclic, bicyclic or tricyclic group of L¹ is directly attached to L²;
where L³ is a divalent 3- to 7-membered monocyclic group, a divalent 5- to 11- or 5- to 12-membered bicyclic group, or a divalent 7- to 16- or 7- to 18-membered tricyclic group, a ring atom of the monocyclic, bicyclic or tricyclic group of L³ is directly attached to a ring atom of the monocyclic, bicyclic or tricyclic group of L⁴, and the same or a different ring atom of the monocyclic, bicyclic or tricyclic group of L³ is directly attached to L²; and
a ring atom of the divalent 3- to 7-membered monocyclic group, divalent 5- to 11- or 5- to 12-membered bicyclic group, or divalent 7- to 16- or 7- to 18-membered tricyclic group of L⁴ is directly attached to the carbon atom of X (i.e. the carbon atom underlined in —C(R²)₂—), and the same or a different ring atom of the monocyclic, bicyclic or tricyclic group of L⁴ is either (i) directly attached to a ring atom of the divalent 3- to 7-membered monocyclic group, divalent 5- to 11- or 5- to 12-membered bicyclic group, or divalent 7- to 16- or 7- to 18-membered tricyclic group of L³, or (ii), where L³ is a bond, directly attached to L².

Where L¹ is a cyclic group, such as a divalent 3- to 7-membered monocyclic group, a divalent 5- to 11- or 5- to 12-membered bicyclic group, or a divalent 7- to 16- or 7- to 18-membered tricyclic group, the ring atom of the cyclic group that is directly attached to the sulfur atom of J may be a nitrogen or a carbon atom. In one embodiment, the ring atom of the cyclic group of L¹ that is directly attached to the sulfur atom of J is a carbon atom.

The ring atom of the divalent 3- to 7-membered monocyclic group, divalent 5- to 11- or 5- to 12-membered bicyclic group, or divalent 7- to 16- or 7- to 18-membered tricyclic group of L⁴ that is directly attached to the carbon atom of X (i.e. the carbon atom underlined in —C(R²)₂—) may be a nitrogen or a carbon atom. Typically, the ring atom of the cyclic group of L⁴ that is directly attached to the carbon atom of X is a carbon atom.

As stated, -J-N(R¹)—C(=Q)-X- and -L¹-L²-L³-L⁴- together form a ring, such that the minimum single ring size that encompasses all or part of each of -J-, —N(R¹)—, —C(=Q)-, —X—, -L¹-, -L²-, -L³- and -L⁴- is from 8 to 30 atoms. Typically, the minimum single ring size that encompasses all or part of each of -J-, —N(R¹)—, —C(=Q)-, —X—, -L¹-, -L²-, -L³- and -L⁴- is from 12 to 24 atoms. More typically, the minimum single ring size that encompasses all or part of each of -J-, —N(R¹)—, —C(=Q)-, —X—, -L¹-, -L²-, -L³- and -L⁴- is from 14 to 20 atoms.

As stated, L¹ is a bond, a divalent 3- to 7-membered monocyclic group, a divalent 5- to 12-membered bicyclic group, or a divalent 7- to 18-membered tricyclic group, any of which may optionally be substituted with one or more monovalent substituents and/or π-bonded substituents. Typically, L¹ is a bond, a divalent 3- to 7-membered monocyclic group, a divalent 5- to 11-membered bicyclic group, or a divalent 7- to 16-membered tricyclic group, any of which may optionally be substituted with one or more monovalent substituents and/or π-bonded substituents. More typically, L¹ is a bond, a divalent 3- to 7-membered monocyclic group, a divalent 7- to 11-membered bicyclic group, or a divalent 9- to 16-membered tricyclic group, any of which may optionally be substituted with one or more monovalent substituents and/or π-bonded substituents.

In one embodiment, L² is a bond.

In one embodiment, where L¹ is a bond, the atom of L² that is directly attached to the sulfur atom of J is a nitrogen or a carbon atom. In a further embodiment, where L¹ is a bond, the atom of L² that is directly attached to the sulfur atom of J is a carbon atom.

In another embodiment, L¹ is a divalent 3- to 7-membered monocyclic group, a divalent 5- to 12-membered bicyclic group, or a divalent 7- to 18-membered tricyclic group, any of which may optionally be substituted with one or more monovalent substituents and/or π-bonded substituents. Typically in such an embodiment, L¹ is a divalent 3- to 7-membered monocyclic group, a divalent 5- to 11-membered bicyclic group, or a divalent 7- to 16-membered tricyclic group, any of which may optionally be substituted with one or more monovalent substituents and/or π-bonded substituents. More typically in such an embodiment, L¹ is a divalent 3- to 7-membered monocyclic group, a divalent 7- to 11-membered bicyclic group, or a divalent 9- to 16-membered tricyclic group, any of which may optionally be substituted with one or more monovalent substituents and/or π-bonded substituents.

In one embodiment, L¹ is a divalent 3- to 7-membered monocyclic group, or a divalent 7- to 11-membered bicyclic group, either of which may optionally be substituted with one or more monovalent substituents and/or π-bonded substituents.

In one aspect of such an embodiment, L¹ is a divalent phenyl, naphthalene, 5- or 6-membered monocyclic heteroaryl, or 8- to 10-membered (e.g. 9- or 10-membered) bicyclic heteroaryl group, any of which may optionally be substituted with one or more monovalent substituents. More typically in such an embodiment, L¹ is a divalent phenyl, or 5- or 6-membered monocyclic heteroaryl group, any of which may optionally be substituted with one or more monovalent substituents.

In another aspect of such an embodiment, L¹ is a divalent fused 7- to 11-membered bicyclic group, wherein a first ring in the bicyclic structure is aromatic and a second ring in the bicyclic structure is non-aromatic, wherein the first ring may optionally be substituted with one or more monovalent substituents, and wherein the second ring may optionally be substituted with one or more monovalent substituents and/or π-bonded substituents. Typically in such an embodiment, the first ring is a 5- or 6-membered ring and the second ring is a 5- or 6-membered ring.

In yet another aspect of such an embodiment, L¹ is a divalent saturated 3- to 7-membered monocyclic group, or a divalent saturated 7- to 11-membered bicyclic group, any of which may optionally be substituted with one or more monovalent substituents and/or π-bonded substituents. For example, L¹ may be a 3- to 7-membered monocyclic cycloalkylene group, a divalent saturated 4- to 7-membered monocyclic heterocyclic group, a 7- to 11-membered bicyclic cycloalkylene group, or a divalent saturated 7- to 11-membered bicyclic heterocyclic group, any of which may optionally be substituted with one or more monovalent substituents and/or π-bonded substituents.

In one embodiment, L¹ is a divalent saturated 3- to 7-membered monocyclic group, which may optionally be substituted with one or more monovalent substituents and/or π-bonded substituents. In one aspect of such an embodiment, L¹ is a divalent saturated 4- to 7-membered monocyclic heterocyclic group (such as a divalent azetidinyl, oxetanyl, thietanyl, pyrrolidinyl, tetrahydrofuranyl, tetrahydrothiophenyl, pyrazolidinyl, imidazolidinyl, oxazolidinyl, isoxazolidinyl, thiazolidinyl, isothiazolidinyl, dioxolanyl, dithiolanyl, piperidinyl, tetrahydropyranyl, thianyl, piperazinyl, morpholinyl, thiomorpholinyl, dioxanyl, dithianyl, azepanyl, diazepanyl, oxepanyl or thiepanyl group), which may optionally be substituted with one or more monovalent substituents and/or π-bonded substituents.

In another embodiment, L¹ is a divalent saturated 7- to 11-membered fused bicyclic group, which may optionally be substituted with one or more monovalent substituents and/or π-bonded substituents. In one aspect of such an embodiment, L¹ is a divalent saturated 7- to 11-membered fused bicyclic heterocyclic group, which may optionally be substituted with one or more monovalent substituents and/or π-bonded substituents.

In yet another embodiment, L¹ is a divalent 5- to 12-membered spiro bicyclic group, which may optionally be substituted with one or more monovalent substituents and/or π-bonded substituents. Typically in such an embodiment, L¹ is a divalent 7- to 11-membered spiro bicyclic group, which may optionally be substituted with one or more monovalent substituents and/or π-bonded substituents. For example, L¹ may be a divalent saturated 7- to 11-membered spiro bicyclic group, which may optionally be substituted with one or more monovalent substituents and/or π-bonded substituents. In one aspect of such an embodiment, L¹ is a divalent saturated 7- to 11-membered spiro bicyclic heterocyclic group, which may optionally be substituted with one or more monovalent substituents and/or π-bonded substituents.

In a further embodiment, L¹ is a divalent 6- to 10-membered bridged bicyclic group, which may optionally be substituted with one or more monovalent substituents and/or π-bonded substituents. For example, L¹ may be a divalent saturated 7- to 9-membered bridged bicyclic group, which may optionally be substituted with one or more monovalent substituents and/or π-bonded substituents. In one aspect of such an embodiment, L¹ is a divalent saturated 7- to 9-membered bridged bicyclic heterocyclic group, which may optionally be substituted with one or more monovalent substituents and/or π-bonded substituents.

As stated, L² is an alkylene, alkenylene or alkynylene group, wherein the alkylene, alkenylene or alkynylene group may be straight-chained or branched, or be or include one or more cyclic groups, wherein one or more carbon atoms in the backbone of the alkylene, alkenylene or alkynylene group may optionally be replaced by one or more heteroatoms independently selected from N, O and S, and wherein the alkylene, alkenylene or alkynylene group may optionally be substituted with one or more monovalent substituents, and/or one or more π-bonded substituents.

As will be understood, where an alkylene, alkenylene or alkynylene group of L² is or includes one or more cyclic groups, the one or more cyclic groups may be monocyclic, bicyclic or polycyclic and selected from cycloalkyl, saturated heterocyclic, cycloalkenyl, partially unsaturated heterocyclic, aryl and heteroaryl groups. Typically, the alkylene, alkenylene or alkynylene group of L² is straight-chained or branched, or is or includes one or two monocyclic groups, or is or includes a single bicyclic group. More typically, the alkylene, alkenylene or alkynylene group of L² is straight-chained or branched, or is or includes a single monocyclic group.

In one embodiment, L² is an alkylene or alkenylene group, wherein the alkylene or alkenylene group may be straight-chained or branched, or be or include one or more cyclic groups, wherein one or more carbon atoms in the backbone of the alkylene or alkenylene group may optionally be replaced by one or more heteroatoms independently selected from N, O and S, and wherein the alkylene or alkenylene group may optionally be substituted with one or more monovalent substituents, and/or one or more π-bonded substituents.

In another embodiment, L² is an alkylene or alkenylene group, wherein the alkylene or alkenylene group may be straight-chained or branched, or include a single cyclic group, wherein one or more carbon atoms in the backbone of the alkylene or alkenylene group may optionally be replaced by one or more heteroatoms independently selected from N, O and S, and wherein the alkylene or alkenylene group may optionally be substituted with one or more monovalent substituents, and/or one or more π-bonded substituents. Typically in such an embodiment, the single cyclic group where present is monocyclic or bicyclic. More typically, the single cyclic group where present is monocyclic. More typically still, the single cyclic group where present is selected from a phenyl, 5- or 6-membered monocyclic heteroaryl, 3- to 7-membered monocyclic cycloalkyl or saturated 4- to 7-membered monocyclic heterocyclic group.

In another embodiment, L² is an alkylene or alkenylene group, wherein the alkylene or alkenylene group is straight-chained or branched, wherein one or more carbon atoms in the backbone of the alkylene or alkenylene group may optionally be replaced by one or more heteroatoms independently selected from N, O and S, and wherein the alkylene or alkenylene group may optionally be substituted with one or more monovalent substituents, and/or one or more π-bonded substituents.

In a further embodiment, L² is an alkylene or alkenylene group, wherein the alkylene or alkenylene group may be straight-chained or branched, or be or include one or more cyclic groups, wherein one or more carbon atoms in the backbone of the alkylene or alkenylene group may optionally be replaced by one or more heteroatoms independently selected from N and O, and wherein the alkylene or alkenylene group may optionally be substituted with one or more monovalent substituents, and/or one or more π-bonded substituents.

In another embodiment, L² is an alkylene or alkenylene group, wherein the alkylene or alkenylene group may be straight-chained or branched, or include a single cyclic group, wherein one or more carbon atoms in the backbone of the alkylene or alkenylene group may optionally be replaced by one or more heteroatoms independently selected from N and O, and wherein the alkylene or alkenylene group may optionally be substituted with one or more monovalent substituents, and/or one or more π-bonded substituents. Typically in such an embodiment, the single cyclic group where present is monocyclic or bicyclic. More typically, the single cyclic group where present is monocyclic. More typically still, the single cyclic group where present is selected from a phenyl, 5- or 6-membered monocyclic heteroaryl, 3- to 7-membered monocyclic cycloalkyl or saturated 4- to 7-membered monocyclic heterocyclic group.

In a further embodiment, L² is an alkylene or alkenylene group, wherein the alkylene or alkenylene group is straight-chained or branched, wherein one or more carbon atoms in the backbone of the alkylene or alkenylene group may optionally be replaced by one or more heteroatoms independently selected from N and O, and wherein the alkylene or alkenylene group may optionally be substituted with one or more monovalent substituents, and/or one or more π-bonded substituents.

In one embodiment, L² is an alkylene group, wherein the alkylene group may be straight-chained or branched, or include a single cyclic group, wherein the alkylene group optionally includes one, two or three heteroatoms independently selected from O and N in its carbon skeleton, and wherein the alkylene group may optionally be substituted with one or more monovalent substituents, and/or one or more π-bonded substituents. As will be understood, in such an embodiment the single cyclic group where present may be a cycloalkyl or a saturated heterocyclic group. Typically in such an embodiment, the single cyclic group where present is monocyclic. More typically, the single cyclic group where present is selected from a 3- to 7-membered monocyclic cycloalkyl or a saturated 4- to 7-membered monocyclic heterocyclic group (such as a divalent azetidinyl, oxetanyl, thietanyl, pyrrolidinyl, tetrahydrofuranyl, tetrahydrothiophenyl, pyrazolidinyl, imidazolidinyl, oxazolidinyl, isoxazolidinyl, thiazolidinyl, isothiazolidinyl, dioxolanyl, dithiolanyl, piperidinyl, tetrahydropyranyl, thianyl, piperazinyl, morpholinyl, thiomorpholinyl, dioxanyl, dithianyl, azepanyl, diazepanyl, oxepanyl or thiepanyl group).

In another embodiment, L² is a straight-chained alkylene group, wherein the straight-chained alkylene group optionally includes one or two heteroatoms independently selected from O and N in its carbon skeleton, and wherein the straight-chained alkylene group may optionally be substituted with one or more monovalent substituents, and/or one or more π-bonded substituents.

Typically, any alkylene, alkenylene or alkynylene group of L² includes at least one heteroatom independently selected from O and N in its carbon skeleton. In one embodiment, the atom of L² that is directly attached to L³ is O or N. In a further embodiment, the atom of L² that is directly attached to L³ is O.

Typically L², including any optional substituents, contains in total from 1 to 5 nitrogen, oxygen and sulfur atoms. More typically L², including any optional substituents, contains in total from 1 to 3 nitrogen, oxygen and sulfur atoms.

In one embodiment, L² contains only atoms selected from the group consisting of hydrogen, halo, carbon, nitrogen and oxygen atoms. Typically in such an embodiment L², including any optional substituents, contains in total from 1 to 5 nitrogen and oxygen atoms. More typically L², including any optional substituents, contains in total from 1 to 3 nitrogen and oxygen atoms.

Typically L², including any optional substituents, contains in total from 1 to 15 carbon atoms. More typically L², including any optional substituents, contains in total from 1 to 8 carbon atoms.

Typically L², including any optional substituents, contains in total from 1 to 20 carbon, nitrogen, oxygen and sulfur atoms. More typically L², including any optional substituents, contains in total from 2 to 15 carbon, nitrogen, oxygen and sulfur atoms. More typically still L², including any optional substituents, contains in total from 2 to 10 carbon, nitrogen, oxygen and sulfur atoms.

Typically, L² has a chain length of from 1 to 15 atoms. More typically, L² has a chain length of from 2 to 12 atoms. More typically still, L² has a chain length of from 2 to 8 atoms. As will be understood, the “chain length” of L² refers to the number of atoms of L² that are bonded to each other in a continuous chain between L¹ and L³, as measured by the shortest route. By way of example, structure (C) has a chain length of 3 atoms, whereas structure (D) has a chain length of 5 atoms:

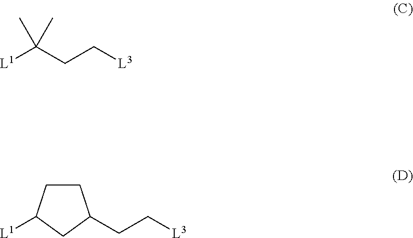

As stated, L³ is a bond, a divalent 3- to 7-membered monocyclic group, a divalent 5- to 12-membered bicyclic group, or a divalent 7- to 18-membered tricyclic group, any of which may optionally be substituted with one or more monovalent substituents and/or π-bonded substituents. Typically, L³ is a bond, a divalent 3- to 7-membered monocyclic group, a divalent 5- to 11-membered bicyclic group, or a divalent 7- to 16-membered tricyclic group, any of which may optionally be substituted with one or more monovalent substituents and/or π-bonded substituents. More typically, L³ is a bond, a divalent 3- to 7-membered monocyclic group, a divalent 7- to 11-membered bicyclic group, or a divalent 9- to 16-membered tricyclic group, any of which may optionally be substituted with one or more monovalent substituents and/or π-bonded substituents.

In one embodiment, L³ is a bond.

In another embodiment, L³ is a divalent 3- to 7-membered monocyclic group, a divalent 5- to 12-membered bicyclic group, or a divalent 7- to 18-membered tricyclic group, any of which may optionally be substituted with one or more monovalent substituents and/or π-bonded substituents. Typically in such an embodiment, L³ is a divalent 3- to 7-membered monocyclic group, a divalent 5- to 11-membered bicyclic group, or a divalent 7- to 16-membered tricyclic group, any of which may optionally be substituted with one or more monovalent substituents and/or π-bonded substituents. More typically in such an embodiment, L³ is a divalent 3- to 7-membered monocyclic group, a divalent 7- to 11-membered bicyclic group, or a divalent 9- to 16-membered tricyclic group, any of which may optionally be substituted with one or more monovalent substituents and/or π-bonded substituents.

In one embodiment, L³ is a divalent 3- to 7-membered monocyclic group, or a divalent 7- to 11-membered bicyclic group, either of which may optionally be substituted with one or more monovalent substituents and/or π-bonded substituents. In one aspect of such an embodiment, L³ is a divalent phenyl, naphthalene, 5- or 6-membered monocyclic heteroaryl, or 8- to 10 membered (e.g. 9- or 10-membered) bicyclic heteroaryl group, any of which may optionally be substituted with one or more monovalent substituents. Typically in such an embodiment, L³ is a divalent phenyl or 5- or 6-membered monocyclic heteroaryl group, any of which may optionally be substituted with one or more monovalent substituents. More typically, L³ is a divalent phenyl or 6-membered monocyclic heteroaryl group, such as a divalent pyridazinyl or divalent pyridinyl group, any of which may optionally be substituted with one or more monovalent substituents.

Typically, where L³ is a divalent 3- to 7-membered monocyclic group, a divalent 5- to 12-membered (e.g. 5- to 11-membered or 7- to 11-membered) bicyclic group, or a divalent 7- to 18-membered (e.g. 7- to 16-membered or 9- to 16-membered) tricyclic group, the atom of L² that is directly attached to L³ is O or N. More typically in such an embodiment, the atom of L² that is directly attached to L³ is O.

As stated, L⁴ is a divalent 3- to 7-membered monocyclic group, a divalent 5- to 12-membered bicyclic group, or a divalent 7- to 18-membered tricyclic group, any of which may optionally be substituted with one or more monovalent substituents and/or π-bonded substituents. Typically, L⁴ is a divalent 3- to 7-membered monocyclic group, a divalent 5- to 11-membered bicyclic group, or a divalent 7- to 16-membered tricyclic group, any of which may optionally be substituted with one or more monovalent substituents and/or π-bonded substituents. More typically, L⁴ is a divalent 3- to 7-membered monocyclic group, a divalent 7- to 11-membered bicyclic group, or a divalent 9- to 16-membered tricyclic group, any of which may optionally be substituted with one or more monovalent substituents and/or π-bonded substituents.

In one embodiment, the ring of the divalent monocyclic, bicyclic or tricyclic group of L⁴ that is directly attached to X is aromatic. For example, L⁴ may be selected from:

(i) a divalent phenyl or 5- or 6-membered heteroaryl group, wherein the divalent phenyl or 5- or 6-membered heteroaryl group may optionally be substituted with one or more monovalent substituents; or

(ii) a divalent 7- to 11-membered bicyclic group, wherein a first ring in the bicyclic structure is aromatic, and a second ring in the bicyclic structure is aromatic or non-aromatic, wherein X is directly attached to a ring atom of the first ring, wherein L³ is directly attached to a ring atom of either the first or the second ring, and wherein the divalent 7- to 11-membered bicyclic group may optionally be substituted with one or more monovalent substituents and/or π-bonded substituents; or

(iii) a divalent 9- to 16-membered tricyclic group, such as a divalent 9- to 16-membered fused tricyclic group, wherein a first ring in the tricyclic structure is aromatic, a second ring in the tricyclic structure is aromatic or non-aromatic, and a third ring in the tricyclic structure is aromatic or non-aromatic, wherein X is directly attached to a ring atom of the first ring, wherein L³ is directly attached to a ring atom of any of the first, second or third rings, and wherein the divalent 9- to 16-membered tricyclic group may optionally be substituted with one or more monovalent substituents and/or π-bonded substituents.

In one embodiment, L⁴ is a divalent 3- to 7-membered monocyclic group, or a divalent 7- to 11-membered bicyclic group, either of which may optionally be substituted with one or more monovalent substituents and/or π-bonded substituents. Typically in such an embodiment, L⁴ is a divalent 3- to 7-membered monocyclic group, or a divalent 7- to 11-membered fused bicyclic group, either of which may optionally be substituted with one or more monovalent substituents and/or π-bonded substituents. More typically in such an embodiment, L⁴ is a divalent 5- or 6-membered monocyclic group, or a divalent 8- to 10-membered fused bicyclic group, either of which may optionally be substituted with one or more monovalent substituents and/or π-bonded substituents. For example, L⁴ may be a phenyl or 5- or 6-membered heteroaryl group, optionally wherein a 5- or 6-membered cyclic group is fused to the phenyl or 5- or 6-membered heteroaryl group, wherein X is directly attached to a ring atom of the phenyl or 5- or 6-membered heteroaryl group, wherein L³ is directly attached to a ring atom of any of the phenyl, 5- or 6-membered heteroaryl or fused 5- or 6-membered cyclic groups, wherein the phenyl or 5- or 6-membered heteroaryl group may optionally be further substituted with one or more monovalent substituents, and wherein the fused 5- or 6-membered cyclic group may optionally be substituted with one or more monovalent substituents and/or π-bonded substituents.

In one aspect of such an embodiment, X and L³ are directly attached to the same ring of L⁴. For example, L⁴ may be a phenyl or 5- or 6-membered heteroaryl group, optionally wherein a 5- or 6-membered cyclic group is fused to the phenyl or 5- or 6-membered heteroaryl group, wherein X is directly attached to a first ring atom of the phenyl or 5- or 6-membered heteroaryl group, wherein L³ is directly attached to a second ring atom of the phenyl or 5- or 6-membered heteroaryl group, wherein the phenyl or 5- or 6-membered heteroaryl group may optionally be further substituted with one or more monovalent substituents, and wherein the fused 5- or 6-membered cyclic group may optionally be substituted with one or more monovalent substituents and/or π-bonded substituents.

Typically, where X and L³ are directly attached to the same ring of L⁴, L³ is not a bond.

Typically, where X and L³ are directly attached to the same ring of L⁴, the ring atom of L⁴ that is directly attached to L³ is at the α-position relative to the ring atom of L⁴ that is directly attached to X. Typically in such an embodiment, the ring to which X and L³ are directly attached is further substituted at the α′-position, typically wherein the substituent at the α′-position comprises at least one carbon atom and/or forms part of a ring structure that is ortho-fused to the ring to which X and L³ are directly attached across the α′,β′ positions. For example, L⁴ may be a divalent phenyl or 5- or 6-membered heteroaryl group, wherein the ring atom of L⁴ that is directly attached to L³ is at the α-position relative to the ring atom of L⁴ that is directly attached to X, wherein either
(i) a 5- or 6-membered cyclic group is fused to the divalent phenyl or 5- or 6-membered heteroaryl group across the α′,β′ positions, wherein the fused 5- or 6-membered cyclic group may optionally be substituted with one or more monovalent substituents and/or π-bonded substituents; or
(ii) the divalent phenyl or 5- or 6-membered heteroaryl group is substituted at the α′-position with a monovalent substituent comprising at least one carbon atom;
   and wherein the divalent phenyl or 5- or 6-membered heteroaryl group may optionally be further substituted with one or more monovalent substituents.

As used herein, the nomenclature α, β, α′, β′ refers to the position of the atoms of a cyclic group, such as L⁴, relative to the specified point of attachment of the cyclic group to the remainder of the molecule. For example, where L⁴ is a divalent 2,3-dihydro-1H-indenyl moiety attached to X at the 4-position and to L³ at the 5-position, the α, β, α′ and β′ positions relative to the ring atom of L⁴ that is directly attached to X are as follows:

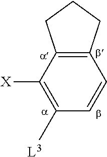

For the avoidance of doubt, where it is stated that a cyclic group, such as a phenyl or a heteroaryl group, is substituted at the α and/or α′ positions, it is to be understood that one or more hydrogen atoms at the α and/or α′ positions respectively are replaced by one or more substituents, such as any optional substituent as defined herein. Unless stated otherwise, the term “substituted” does not include the replacement of one or more ring carbon atoms by one or more ring heteroatoms.

In another embodiment, L⁴ is a divalent 7- to 11-membered bicyclic group, or a divalent 9- to 16-membered tricyclic group, either of which may optionally be substituted with one or more monovalent substituents and/or π-bonded substituents. Typically in such an embodiment, L⁴ is a divalent 7- to 11-membered fused bicyclic group, or a divalent 9- to 16-membered fused tricyclic group, either of which may optionally be substituted with one or more monovalent substituents and/or π-bonded substituents. More typically in such an embodiment, L⁴ is a divalent 8- to 10-membered fused bicyclic group or a divalent 11- to 14-membered fused tricyclic group, either of which may optionally be substituted with one or more monovalent substituents and/or π-bonded substituents. For example, L⁴ may be a phenyl or 5- or 6-membered heteroaryl group, wherein a first 5- or 6-membered cyclic group is fused to the phenyl or 5- or 6-membered heteroaryl group, optionally wherein a second 5- or 6-membered cyclic group is fused to the phenyl or 5- or 6-membered heteroaryl group, wherein X is directly attached to a ring atom of the phenyl or 5- or 6-membered heteroaryl group, wherein L³ is directly attached to a ring atom of any of the phenyl, 5- or 6-membered heteroaryl or fused 5- or 6-membered cyclic groups, wherein the phenyl or 5- or 6-membered heteroaryl group may optionally be further substituted with one or more monovalent substituents, and wherein the fused 5- or 6-membered cyclic groups may optionally be substituted with one or more monovalent substituents and/or π-bonded substituents.

In one aspect of such an embodiment, X and L³ are directly attached to different rings within the bicyclic or tricyclic group. For example, L⁴ may be a phenyl or 5- or 6-membered heteroaryl group, wherein a ring atom of the phenyl or 5- or 6-membered heteroaryl group is directly attached to X, wherein a first 5- or 6-membered cyclic group is fused to the phenyl or 5- or 6-membered heteroaryl group, wherein a ring atom of the first fused 5- or 6-membered cyclic group is directly attached to L³, wherein optionally a second 5- or 6-membered cyclic group is fused to the phenyl or 5- or 6-membered heteroaryl group, wherein the phenyl or 5- or 6-membered heteroaryl group may optionally be further substituted with one or more monovalent substituents, and wherein either fused 5- or 6-membered cyclic group may optionally be substituted with one or more monovalent substituents and/or π-bonded substituents.

Typically, where X and L³ are directly attached to different rings within the divalent bicyclic or tricyclic group of L⁴, L³ is a bond, such that X and L² are directly attached to different rings within the bicyclic or tricyclic group of L⁴. Typically, X is directly attached to a ring atom of a first ring of the bicyclic or tricyclic group, a second ring of the bicyclic or tricyclic group is ortho-fused to the first ring across the α,β positions of the first ring, relative to the ring atom of the first ring that is directly attached to X, and L³ (or L² where L³ is a bond) is directly attached to a ring atom of the second ring that is not also a ring atom of the first ring. Typically, the ring atom of the second ring that is directly attached to L³ (or directly attached to L² where L³ is a bond) is also directly attached to the ring atom at the α-position of the first ring. For example, L⁴ may be a phenyl or 5- or 6-membered heteroaryl group, wherein a ring atom of the phenyl or 5- or 6-membered heteroaryl group is directly attached to X, wherein a first 5- or 6-membered cyclic group is fused to the phenyl or 5- or 6-membered heteroaryl group across the α,β positions of the phenyl or 5- or 6-membered heteroaryl group, relative to the ring atom that is directly attached to X, wherein a ring atom of the first fused 5- or 6-membered cyclic group is directly attached to L², wherein either
(i) a second 5- or 6-membered cyclic group is fused to the phenyl or 5- or 6-membered heteroaryl group across the α′,β′ positions; or
(ii) the phenyl or 5- or 6-membered heteroaryl group is substituted at the α′-position with a monovalent substituent comprising at least one carbon atom;
   wherein the phenyl or 5- or 6-membered heteroaryl group may optionally be further substituted with one or two monovalent substituents, and wherein either fused 5- or 6-membered cyclic group may optionally be substituted with one or more monovalent substituents and/or π-bonded substituents. Typically, the ring atom of the first fused 5- or 6-membered cyclic group that is directly attached to L² is also directly attached to the ring atom at the α-position of the phenyl or 5- or 6-membered heteroaryl group.

Where L¹, L², L³ or L⁴ is substituted with one or more monovalent substituents, the monovalent substituents may be independently selected from any monovalent substituent as discussed above. Typically, where any moiety selected from L¹, L², L³ or L⁴ is substituted with one or more monovalent substituents, the moiety is substituted with one, two, three or four monovalent substituents. More typically, where any moiety selected from L¹, L², L³ or L⁴ is substituted with one or more monovalent substituents, the moiety is substituted with one, two, or three monovalent substituents. In one embodiment, where L¹, L², L³ or L⁴ is substituted with one or more monovalent substituents, each monovalent substituent is independently selected from a halo, C₁-C₆ alkyl, C₁-C₆ haloalkyl, C₂-C₆ alkenyl, C₂-C₆ haloalkenyl, —R¹¹-R¹², —R¹¹—CN, —R¹¹—N₃, —R¹¹—NO₂, —R¹¹—N(R¹³)₂, —R¹¹—OR¹³, —R¹¹—COR¹³, —R¹¹—COOR¹³, —R¹¹—CON(R¹³)₂, —R¹¹—C(═NR¹³)R¹³, —R¹¹—C(═NR¹³)N(R¹³)₂, —R¹¹—C(═NOR¹³)R¹³, —R¹¹—SO₂R¹³ or —R¹¹—SO₂N(R¹³)₂ group, wherein:
each R¹¹ is independently selected from a bond, or a C₁-C₄ alkylene group, wherein the C₁-C₄ alkylene group may be straight-chained or branched, or be or include a C₃-C₄ cycloalkylene group, and wherein the C₁-C₄ alkylene group may optionally be substituted with one or more halo groups;
each R¹² is independently selected from a 3- to 6-membered cyclic group, wherein the 3- to 6-membered cyclic group may optionally be substituted with one or more halo groups and/or one, two or three substituents independently selected from —CN, —NO₂, —R¹⁴, —OH, —OR¹⁴, —NH₂, —NHR¹⁴ and —N(R¹⁴)₂;
each R¹³ is independently selected from hydrogen or a C₁-C₆ alkyl, C₁-C₆ haloalkyl, C₂-C₆ alkenyl, C₂-C₆ haloalkenyl, or 3- to 6-membered cyclic group, wherein the 3- to 6-membered cyclic group may optionally be substituted with one or more halo groups and/or one, two or three substituents independently selected from —CN, —NO₂, —R¹⁴, —OH, —OR¹⁴, —NH₂, —NHR¹⁴ and —N(R¹⁴)₂, or any two R¹³ attached to the same nitrogen atom may together form a C₂-C₅ alkylene or C₂-C₅ haloalkylene group; and
each R¹⁴ is independently selected from a C₁-C₄ alkyl or C₁-C₄ haloalkyl group.

Where L¹, L², L³ or L⁴ is substituted with one or more π-bonded substituents, the π-bonded substituents may be independently selected from any π-bonded substituent as discussed above. Typically, where any moiety selected from L¹, L², L³ or L⁴ is substituted with one or more π-bonded substituents, the moiety is substituted with one or two π-bonded substituents. More typically, where any moiety selected from L¹, L², L³ or L⁴ is substituted with one or more π-bonded substituents, the moiety is substituted with a single π-bonded substituent. In one embodiment, where L¹, L², L³ or L⁴ is substituted with one or more π-bonded substituents, each π-bonded substituent is independently selected from ═O or ═NR¹³, wherein R¹³ is as defined above.

In one embodiment of the first aspect of the invention, the compound has the formula (Ib):

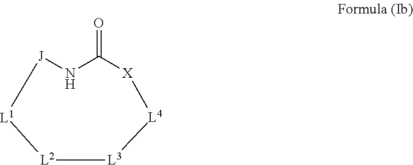

wherein:
J is —SO—, —SO₂— or —SO(═NH)—;
X is —CH₂—;
J-NH—C(═O)—X- and -L¹-L²-L³-L⁴- together form a ring, such that the minimum single ring size that encompasses all or part of each of -J-NH—C(═O)—X—, -L¹-, -L²-, -L³- and -L⁴- is from 8 to 30 atoms; and
L¹, L², L³ and L⁴ are as previously defined.

Typically, where the compound has the formula (Ib), J is —SO₂—.

Typically, the minimum single ring size that encompasses all or part of each of -J-NH—C(═O)—X—, -L¹-, -L²-, -L³- and -L⁴- is from 12 to 24 atoms. More typically, the minimum single ring size that encompasses all or part of each of -J-NH—C(═O)—X—, -L¹-, -L²-, -L³- and -L⁴- is from 14 to 20 atoms.

In a first exemplary embodiment, where the compound has the formula (Ib):
L¹ is a bond, a divalent 3- to 7-membered monocyclic group, or a divalent 7- to 11-membered bicyclic group, wherein the divalent 3- to 7-membered monocyclic group or divalent 7- to 11-membered bicyclic group may optionally be substituted with one or more halo groups and/or one or more oxo (═O) groups and/or one or more substituents R^{L};
L² is an alkylene or alkenylene group, wherein the alkylene or alkenylene group may be straight-chained or branched, or be or include one or more cyclic groups, wherein one or more carbon atoms in the backbone of the alkylene or alkenylene group may optionally be replaced by one or more heteroatoms independently selected from N and O, and wherein the alkylene or alkenylene group may optionally be substituted with one or more halo groups;
L³ is a divalent phenyl or 5- or 6-membered heteroaryl group, wherein the divalent phenyl or 5- or 6-membered heteroaryl group may optionally be substituted with one or more halo groups and/or one or more substituents R^{L};
L⁴ is a divalent phenyl or 5- or 6-membered heteroaryl group, wherein the divalent phenyl or 5- or 6-membered heteroaryl group may optionally be substituted with one or more halo groups and/or one or more substituents R^{L};
the ring atom of L⁴ that is directly attached to L³ is at the α-position relative to the ring atom of L⁴ that is directly attached to X;
each R^{L} is independently selected from a C₁-C₆ alkyl, C₁-C₆ haloalkyl, C₂-C₆ alkenyl, C₂-C₆ haloalkenyl, —R¹¹-R¹², —R¹¹—CN, —R¹¹—N₃, —R¹¹—NO₂, —R¹¹—N(R¹³)₂, —R¹¹—OR¹³, —R¹¹—COR¹³, —R¹¹—COOR¹³, —R¹¹—CON(R¹³)₂, —R¹¹—C(═NR¹³)R¹³, —R¹¹—C(═NR¹³)N(R¹³)₂, —R¹¹—C(═NOR¹³)R¹³, —R¹¹—SO₂R¹³ or —R¹¹—SO₂N(R¹³)₂ group, and/or any two R^{L} attached to the same divalent phenyl or 5- or 6-membered heteroaryl group of L³ or L⁴ may, together with the atoms of the divalent phenyl or 5- or 6-membered heteroaryl group to which they are attached, form a fused 5- or 6-membered cyclic group, wherein the fused 5- or 6-membered cyclic group may optionally be substituted with one or more halo groups and/or one or two oxo (═O) groups and/or one, two or three substituents independently selected from a C₁-C₆ alkyl, C₁-C₆ haloalkyl, C₂-C₆ alkenyl, C₂-C₆ haloalkenyl, —R¹¹-R¹², —R¹¹—CN, —R¹¹—N₃, —R¹¹—NO₂, —R¹¹—N(R¹³)₂, —R¹¹—OR¹³, —R¹¹—COR¹³, —R¹¹—COOR¹³, —R¹¹—CON(R¹³)₂, —R¹¹—C(═NR¹³)R¹³, —R¹¹—C(═NR¹³)N(R¹³)₂, —R¹¹—C(═NOR¹³)R¹³, —R¹¹—SO₂R¹³ or —R¹¹—SO₂N(R¹³)₂ group; and
R¹¹, R¹² and R¹³ are as previously defined.

Typically in accordance with the first exemplary embodiment, each R^{L} is independently selected from a C₁-C₆ alkyl, C₁-C₆ haloalkyl, C₂-C₆ alkenyl, C₂-C₆ haloalkenyl, —R¹⁵-R¹⁶, —R¹⁵—CN, —R¹⁵—N(R¹⁷)₂, —R¹⁵—OR¹⁷, —R¹⁵—COR¹⁷, —R¹⁵—COOR¹⁷, —R¹⁵—CON(R¹⁷)₂, —R¹⁵—C(═NR¹⁷)R¹⁷, —R¹⁵—C(═NR¹⁷)N(R¹⁷)₂, —R¹⁵—C(═NOR¹⁷)R¹⁷, —R¹⁵—SO₂R¹⁷ or —R¹⁵—SO₂N(R¹⁷)₂ group, and/or any two R^{L} attached to the same divalent phenyl or 5- or 6-membered heteroaryl group of L³ or L⁴ may, together with the atoms of the divalent phenyl or 5- or 6-membered heteroaryl group to which they are attached, form a fused 5- or 6-membered cyclic group, wherein the fused 5- or 6-membered cyclic group may optionally be substituted with one or more halo groups and/or one or two oxo (═O) groups and/or one, two or three substituents independently selected from a C₁-C₆ alkyl, C₁-C₆ haloalkyl, C₂-C₆ alkenyl, C₂-C₆ haloalkenyl, —R¹⁵-R¹⁶, —R¹⁵—CN, —R¹⁵—N(R¹⁷)₂, —R¹⁵—OR¹⁷, —R¹⁵—COR¹⁷, —R¹⁵—COOR¹⁷, —R¹⁵—CON(R¹⁷)₂, —R¹⁵—C(═NR¹⁷)R¹⁷, —R¹⁵—C(═NR¹⁷)N(R¹⁷)₂, —R¹⁵—C(═NOR¹⁷)R¹⁷, —R¹⁵—SO₂R¹⁷ or —R¹⁵—SO₂N(R¹⁷)₂ group, wherein:
each R¹⁵ is independently selected from a bond, or a C₁-C₄ alkylene group, wherein the C₁-C₄ alkylene group may be straight-chained or branched, or be or include a C₃-C₄ cycloalkylene group, and wherein the C₁-C₄ alkylene group may optionally be substituted with one or more halo groups;
each R¹⁶ is independently selected from a 3- to 6-membered cyclic group, wherein the 3- to 6-membered cyclic group may optionally be substituted with one or more halo groups and/or one, two or three substituents independently selected from —CN, —R¹⁸, —OH, —OR¹⁸, —NH₂, —NHR¹⁸ and —N(R¹⁸)₂;
each R¹⁷ is independently selected from hydrogen or a C₁-C₆ alkyl, C₁-C₆ haloalkyl, C₂-C₆ alkenyl, C₂-C₆ haloalkenyl, or 3- to 6-membered cyclic group, wherein the 3- to 6-membered cyclic group may optionally be substituted with one or more halo groups and/or one, two or three substituents independently selected from —CN, —R¹⁸, —OH, —OR¹⁸, —NH₂, —NHR¹⁸ and —N(R¹⁸)₂, or any two R¹⁷ attached to the same nitrogen atom may together form a C₂-C₅ alkylene or C₂-C₅ haloalkylene group; and
each R¹⁸ is independently selected from a C₁-C₄ alkyl or C₁-C₄ haloalkyl group.

In one aspect of the first exemplary embodiment, L¹ is a bond.

In another aspect of the first exemplary embodiment, L¹ is a divalent 3- to 7-membered monocyclic group, or a divalent 7- to 11-membered bicyclic group, wherein the divalent 3- to 7-membered monocyclic group or divalent 7- to 11-membered bicyclic group may optionally be substituted with one or more halo groups and/or one or more oxo (═O) groups and/or one or more substituents R^{L}.

In one aspect of the first exemplary embodiment, L¹ is a divalent phenyl, or 5- or 6-membered heteroaryl group, wherein the divalent phenyl or 5- or 6-membered heteroaryl group may optionally be substituted with one or more halo groups and/or one or more substituents R^{L}. Typically, where L¹ is a divalent phenyl, or 5- or 6-membered heteroaryl group, it is unsubstituted or substituted with one or more halo groups and/or one or two substituents R^{L}.

In another aspect of the first exemplary embodiment, L¹ is a divalent phenyl, or 5- or 6-membered heteroaryl group, wherein the divalent phenyl or 5- or 6-membered heteroaryl group is ortho-fused to a 5- or 6-membered cyclic group, wherein the divalent phenyl or 5- or 6-membered heteroaryl group may optionally be further substituted with one or two substituents independently selected from halo groups and R^{L}, and wherein the fused 5- or 6-membered cyclic group may optionally be substituted with one or more halo groups and/or one or more oxo (═O) groups and/or one or more substituents R^{L}. Typically, the divalent phenyl or 5- or 6-membered heteroaryl group is (aside from the fused 5- or 6-membered cyclic group) unsubstituted or further substituted with one or two halo groups and/or a single substituent R^{L}. Typically, the fused 5- or 6-membered cyclic group is unsubstituted or substituted with one or more halo groups and/or one or two substituents independently selected from oxo (═O) and R^{L}. In one embodiment, the fused 5- or 6-membered cyclic group is non-aromatic, such as a fused non-aromatic 5- or 6-membered heterocyclic group. In another embodiment, the fused 5- or 6-membered cyclic group is aromatic, such as a fused 5- or 6-membered heteroaryl group.

In another aspect of the first exemplary embodiment, L¹ is a divalent saturated 4- to 7-membered monocyclic heterocyclic group, wherein the divalent saturated 4- to 7-membered monocyclic heterocyclic group may optionally be substituted with one or more halo groups and/or one or more oxo (═O) groups and/or one or more substituents R^{L}. For example, L¹ may be a divalent saturated 4- to 7-membered monocyclic heterocyclic group, wherein the divalent saturated 4- to 7-membered monocyclic heterocyclic group includes one or two heteroatoms independently selected from nitrogen and oxygen in its ring structure, and wherein the divalent saturated 4- to 7-membered monocyclic heterocyclic group may optionally be substituted with one or more halo groups and/or one or more oxo (═O) groups and/or one or more substituents R^{L}. Typically, the divalent saturated 4- to 7-membered monocyclic heterocyclic group includes at least one nitrogen atom in its ring structure. For example, L¹ may be selected from a divalent azetidinyl, pyrrolidinyl, piperidinyl, piperazinyl or morpholinyl group, any of which may optionally be substituted with one or more halo groups and/or one or more oxo (═O) groups and/or one or more substituents R^{L}. Typically, where L¹ is a divalent saturated 4- to 7-membered monocyclic heterocyclic group, such as a divalent azetidinyl, pyrrolidinyl, piperidinyl, piperazinyl or morpholinyl group, it is unsubstituted or substituted with one or more halo groups and/or one or two oxo (═O) groups and/or one or two substituents R^{L}. In one embodiment, where the divalent saturated 4- to 7-membered monocyclic heterocyclic group includes at least one nitrogen atom in its ring structure, the ring atom of L¹ that is directly attached to the sulfur atom of J is a nitrogen atom. Typically, where L¹ is a divalent saturated 4- to 7-membered monocyclic heterocyclic group, the ring atom of L¹ that is directly attached to L² is at the α-, β- or γ-position relative to the ring atom of L¹ that is directly attached to the sulfur atom of J. In one embodiment, where L¹ is a divalent saturated 4- to 7-membered monocyclic heterocyclic group, the ring atom of L¹ that is directly attached to L² is at the β-position relative to the ring atom of L¹ that is directly attached to the sulfur atom of J.

Typically in accordance with the first exemplary embodiment, L² contains in total (i.e. including any optional substituents) from 2 to 15 carbon, nitrogen and oxygen atoms. More typically, L² contains in total from 2 to 10 carbon, nitrogen and oxygen atoms. Typically, L² includes at least one heteroatom independently selected from O and N in its carbon skeleton. Typically, L² contains in total from 1 to 3 nitrogen and oxygen atoms. Typically, the atom of L² that is directly attached to L³ is O or N. More typically, the atom of L² that is directly attached to L³ is O.

Typically in accordance with the first exemplary embodiment, L² has a chain length of from 2 to 12 atoms. More typically, L² has a chain length of from 2 to 8 atoms.

In one aspect of the first exemplary embodiment, L² is an alkylene or alkenylene group, wherein the alkylene or alkenylene group may be straight-chained or branched, or include a single monocyclic group, wherein one or more carbon atoms in the backbone of the alkylene or alkenylene group may optionally be replaced by one or more heteroatoms independently selected from N and O, wherein the alkylene or alkenylene group may optionally be substituted with one or more halo groups, and wherein L² contains in total from 2 to 15 carbon, nitrogen and oxygen atoms. Typically, the single monocyclic group where present is selected from a phenyl, 5- or 6-membered monocyclic heteroaryl, 3- to 7-membered monocyclic cycloalkyl or saturated 4- to 7-membered monocyclic heterocyclic group.

In another aspect of the first exemplary embodiment, L² is an alkylene group, wherein the alkylene group may be straight-chained or branched, or include a single monocyclic group, wherein the alkylene group includes one, two or three heteroatoms independently selected from O and N in its carbon skeleton, wherein the alkylene group may optionally be substituted with one or more halo groups, and/or one or two oxo (═O) groups, and wherein L² contains in total from 2 to 15 carbon, nitrogen and oxygen atoms. Typically, the single monocyclic group where present is selected from a 3- to 7-membered monocyclic cycloalkyl or saturated 4- to 7-membered monocyclic heterocyclic group.

In one aspect of the first exemplary embodiment, the divalent phenyl or 5- or 6-membered heteroaryl group of L⁴ is substituted at the α′-position, relative to the ring atom of L⁴ that is directly attached to X, with a substituent R^{L}, wherein R^{L} is as defined above. Typically, the substituent at the α′-position is selected from a C₁-C₆ alkyl, C₁-C₆ haloalkyl, C₂-C₆ alkenyl, C₂-C₆ haloalkenyl, —R¹⁵-R¹⁶, —R¹⁵—CN, —R¹⁵—N(R¹⁷)₂, —R¹⁵—OR¹⁷, —R¹⁵—COR¹⁷, —R¹⁵—COOR¹⁷ or —R¹⁵—CON(R¹⁷)₂ group, wherein R¹⁵, R¹⁶ and R¹⁷ are as previously defined. More typically, the substituent at the α′-position is selected from a C₁-C₆ alkyl, C₁-C₆ haloalkyl, C₂-C₆ alkenyl, C₂-C₆ haloalkenyl, or 3- to 6-membered cyclic group, wherein the 3- to 6-membered cyclic group may optionally be substituted with one or more halo groups.

In another aspect of the first exemplary embodiment, the divalent phenyl or 5- or 6-membered heteroaryl group of L⁴ is ortho-fused to a 5- or 6-membered cyclic group across the α′,ρ′-positions, relative to the ring atom of L⁴ that is directly attached to X, wherein the ortho-fused 5- or 6-membered cyclic group is optionally substituted with one or more halo groups and/or one or two oxo (═O) groups and/or one, two or three substituents independently selected from a C₁-C₆ alkyl, C₁-C₆ haloalkyl, C₂-C₆ alkenyl, C₂-C₆ haloalkenyl, —R¹¹-R¹², —R¹¹—CN, —R¹¹—N₃, —R¹¹—NO₂, —R¹¹—N(R¹³)₂, —R¹¹—OR¹³, —R¹¹—COR¹³, —R¹¹—COOR¹³, —R¹¹—CON(R¹³)₂, —R¹¹—C(═NR¹³)R¹³, —R¹¹—C(═NR¹³)N(R¹³)₂, —R¹¹—C(═NOR¹³)R¹³, —R¹¹—SO₂R¹³ or —R¹¹—SO₂N(R¹³)₂ group, wherein R¹¹, R¹² and R¹³ are as previously defined. Typically, the ortho-fused 5- or 6-membered cyclic group is non-aromatic. For example, the ortho-fused 5- or 6-membered cyclic group may be an ortho-fused 5- or 6-membered cycloalkyl group or an ortho-fused non-aromatic 5- or 6-membered heterocyclic group. Typically, the ortho-fused 5- or 6-membered cyclic group is unsubstituted or is substituted with one or more halo groups and/or one oxo (═O) group and/or one, two or three substituents independently selected from a C₁-C₆ alkyl, C₁-C₆ haloalkyl, C₂-C₆ alkenyl, C₂-C₆ haloalkenyl, —R¹⁵-R¹⁶, —R¹⁵—CN, —R¹⁵—N(R¹⁷)₂, —R¹⁵—OR¹⁷, —R¹⁵—COR¹⁷, —R¹⁵—COOR¹⁷, —R¹⁵—CON(R¹⁷)₂, —R¹⁵—C(═NR¹⁷)R¹⁷, —R¹⁵—C(═NR¹⁷)N(R¹⁷)₂, —R¹⁵—C(═NOR¹⁷)R¹⁷, —R¹⁵—SO₂R¹⁷ or —R¹⁵—SO₂N(R¹⁷)₂ group, wherein R¹⁵, R¹⁶ and R¹⁷ are as previously defined. More typically, the ortho-fused 5- or 6-membered cyclic group is unsubstituted or is substituted with one or more halo groups and/or one oxo (═O) group and/or one, two or three substituents independently selected from a —OH, —CN, C₁-C₄ alkyl, C₁-C₄ haloalkyl, —O(C₁-C₄ alkyl) or —O(C₁-C₄ haloalkyl) group. More typically still, the ortho-fused 5- or 6-membered cyclic group is unsubstituted or substituted with one or more halo groups.

As will be understood, in either of the above two aspects of the first exemplary embodiment, the divalent phenyl or 5- or 6-membered heteroaryl group of L⁴ may optionally be further substituted with one or more halo groups and/or one or more further substituents R^{L}. Typically, the divalent phenyl or 5- or 6-membered heteroaryl group of L⁴ may optionally be further substituted with one or more halo groups and/or one or two substituents each independently selected from a —CN, methyl, halomethyl, —OC(R¹⁹)₃ or —C(R¹⁹)₂—OC(R¹⁹)₃ group, wherein each R¹⁹ is independently selected from hydrogen or a halo group. More typically, the divalent phenyl or 5- or 6-membered heteroaryl group of L⁴ may optionally be further substituted with one or more halo groups and/or one or two substituents each independently selected from a —CN, methyl, halomethyl, —OMe or —O-(halomethyl) group.

In a second exemplary embodiment, where the compound has the formula (Ib):
L¹ is a bond or a divalent phenyl or 5- or 6-membered heteroaryl group;
L² is an alkylene or alkenylene group, wherein one or more carbon atoms in the backbone of the alkylene or alkenylene group may optionally be replaced by one or more heteroatoms independently selected from N and O, and wherein the alkylene or alkenylene group may optionally be substituted with one or more halo groups;
L³ is a divalent phenyl or 5- or 6-membered heteroaryl group;
L⁴ is a divalent phenyl or 5- or 6-membered heteroaryl group;
the ring atom of L⁴ that is directly attached to L³ is at the α-position relative to the ring atom of L⁴ that is directly attached to X;
any divalent phenyl or 5- or 6-membered heteroaryl group may optionally be substituted with one or more halo groups and/or one or more substituents R^{L}, wherein each R^{L} is independently selected from a C₁-C₆ alkyl, C₁-C₆ haloalkyl, C₂-C₆ alkenyl, C₂-C₆ haloalkenyl, —R¹¹-R¹², —R¹¹—CN, —R¹¹—N₃, —R¹¹—NO₂, —R¹¹—N(R¹³)₂, —R¹¹—OR¹³, —R¹¹—COR¹³, —R¹¹—COOR¹³, —R¹¹—CON(R¹³)₂, —R¹¹—C(═NR¹³)R¹³, —R¹¹—C(═NR¹³)N(R¹³)₂, —R¹¹—C(═NOR¹³)R¹³, —R¹¹—SO₂R¹³ or —R¹¹—SO₂N(R¹³)₂ group, and/or any two R^{L} attached to the same divalent phenyl or 5- or 6-membered heteroaryl group may, together with the atoms of the divalent phenyl or 5- or 6-membered heteroaryl group to which they are attached, form a fused 5- or 6-membered cyclic group, wherein the fused 5- or 6-membered cyclic group may optionally be substituted with one or more halo groups and/or one, two or three substituents independently selected from a C₁-C₆ alkyl, C₁-C₆ haloalkyl, C₂-C₆ alkenyl, C₂-C₆ haloalkenyl, —R¹¹-R¹², —R¹¹—CN, —R¹¹—N₃, —R¹¹—NO₂, —R¹¹—N(R¹³)₂, —R¹¹—OR¹³, —R¹¹—COR¹³, —R¹¹—COOR¹³, —R¹¹—CON(R¹³)₂, —R¹¹—C(═NR¹³)R¹³, —R¹¹—C(═NR¹³)N(R¹³)₂, —R¹¹—C(═NOR¹³)R¹³, —R¹¹—SO₂R¹³ or —R¹¹—SO₂N(R¹³)₂ group; and
R¹¹, R¹² and R¹³ are as previously defined.

Typically in accordance with the second exemplary embodiment, each R^{L} is independently selected from a C₁-C₆ alkyl, C₁-C₆ haloalkyl, C₂-C₆ alkenyl, C₂-C₆ haloalkenyl, —R¹⁵-R¹⁶, —R¹⁵—CN, —R¹⁵—N(R¹⁷)₂, —R¹⁵—OR¹⁷, —R¹⁵—COR¹⁷, —R¹⁵—COOR¹⁷, —R¹⁵—CON(R¹⁷)₂, —R¹⁵—C(═NR¹⁷)R¹⁷, —R¹⁵—C(═NR¹⁷)N(R¹⁷)₂, —R¹⁵—C(═NOR¹⁷)R¹⁷, —R¹⁵—SO₂R¹⁷ or —R¹⁵—SO₂N(R¹⁷)₂ group, and/or any two R^{L} attached to the same divalent phenyl or 5- or 6-membered heteroaryl group may, together with the atoms of the divalent phenyl or 5- or 6-membered heteroaryl group to which they are attached, form a fused 5- or 6-membered cyclic group, wherein the fused 5- or 6-membered cyclic group may optionally be substituted with one or more halo groups and/or one, two or three substituents independently selected from a C₁-C₆ alkyl, C₁-C₆ haloalkyl, C₂-C₆ alkenyl, C₂-C₆ haloalkenyl, —R¹⁵-R¹⁶, —R¹⁵—CN, —R¹⁵—N(R¹⁷)₂, —R¹⁵—OR¹⁷, —R¹⁵—COR¹⁷, —R¹⁵—COOR¹⁷, —R¹⁵—CON(R¹⁷)₂, —R¹⁵—C(═NR¹⁷)R¹⁷, —R¹⁵—C(═NR¹⁷)N(R¹⁷)₂, —R¹⁵—C(═NOR¹⁷)R¹⁷, —R¹⁵—SO₂R¹⁷ or —R¹⁵—SO₂N(R¹⁷)₂ group, wherein R¹⁵, R¹⁶ and R¹⁷ are as previously defined.

In one aspect of the second exemplary embodiment, L¹ is a bond.

In another aspect of the second exemplary embodiment, L¹ is a divalent phenyl or 5- or 6-membered heteroaryl group, wherein the divalent phenyl or 5- or 6-membered heteroaryl group may optionally be substituted with one or more halo groups and/or one or more substituents R^{L}, as set out above. Typically, where L¹ is a divalent phenyl, or 5- or 6-membered heteroaryl group, it is unsubstituted or substituted with one or more halo groups and/or one or two substituents R^{L}.

As will be understood, in accordance with the second exemplary embodiment the alkylene or alkenylene group of L² may be straight-chained or branched. Typically in such an embodiment L² contains in total (i.e. including any optional substituents) from 2 to 15 carbon, nitrogen and oxygen atoms. More typically, L² contains in total from 2 to 10 carbon, nitrogen and oxygen atoms. Typically, L² includes at least one heteroatom independently selected from O and N in its carbon skeleton. Typically, L² contains in total from 1 to 3 nitrogen and oxygen atoms. Typically, the atom of L² that is directly attached to L³ is O or N. More typically, the atom of L² that is directly attached to L³ is O.

Typically in accordance with the second exemplary embodiment, L² has a chain length of from 2 to 12 atoms. More typically, L² has a chain length of from 2 to 8 atoms.

In one aspect of the second exemplary embodiment, L² is an alkylene group, wherein the alkylene group may be straight-chained or branched, wherein the alkylene group includes one, two or three heteroatoms independently selected from O and N in its carbon skeleton, wherein the alkylene group may optionally be substituted with one or more halo groups, and/or one or two oxo (═O) groups, and wherein L² contains in total from 2 to 15 carbon, nitrogen and oxygen atoms.

In one aspect of the second exemplary embodiment, the divalent phenyl or 5- or 6-membered heteroaryl group of L⁴ is substituted at the α′-position, relative to the ring atom of L⁴ that is directly attached to X, with a substituent R^{L}, wherein R^{L} is as defined above. Typically, the substituent at the α′-position is selected from a C₁-C₆ alkyl, C₁-C₆ haloalkyl, C₂-C₆ alkenyl, C₂-C₆ haloalkenyl, —R¹¹-R¹², —R¹¹—CN, —R¹¹—N(R¹³)₂, —R¹¹—OR¹³, —R¹¹—COR¹³, —R¹¹—COOR¹³ or —R¹¹—CON(R¹³)₂ group, wherein R¹¹, R¹² and R¹³ are as previously defined. More typically, the substituent at the α′-position is selected from a C₁-C₆ alkyl, C₁-C₆ haloalkyl, C₂-C₆ alkenyl, C₂-C₆ haloalkenyl, —R¹⁵-R¹⁶, —R¹⁵—CN, —R¹⁵—N(R¹⁷)₂, —R¹⁵—OR¹⁷, —R¹⁵—COR¹⁷, —R¹⁵—COOR¹⁷ or —R¹⁵—CON(R¹⁷)₂ group, wherein R¹⁵, R¹⁶ and R¹⁷ are as previously defined. More typically still, the substituent at the α′-position is selected from a C₁-C₆ alkyl, C₁-C₆ haloalkyl, C₂-C₆ alkenyl, C₂-C₆ haloalkenyl, or 3- to 6-membered cyclic group, wherein the 3- to 6-membered cyclic group may optionally be substituted with one or more halo groups.

In another aspect of the second exemplary embodiment, the divalent phenyl or 5- or 6-membered heteroaryl group of L⁴ is ortho-fused to a 5- or 6-membered cyclic group across the α′,β′-positions, relative to the ring atom of L⁴ that is directly attached to X, wherein the ortho-fused 5- or 6-membered cyclic group is optionally substituted with one or more halo groups and/or one, two or three substituents independently selected from a C₁-C₆ alkyl, C₁-C₆ haloalkyl, C₂-C₆ alkenyl, C₂-C₆ haloalkenyl, —R¹¹-R¹², —R¹¹—CN, —R¹¹—N₃, —R¹¹—NO₂, —R¹¹—N(R¹³)₂, —R¹¹—OR¹³, —R¹¹—COR¹³, —R¹¹—COOR¹³, —R¹¹—CON(R¹³)₂, —R¹¹—C(═NR¹³)R¹³, —R¹¹—C(═NR¹³)N(R¹³)₂, —R¹¹—C(═NOR¹³)R¹³, —R¹¹—SO₂R¹³ or —R¹¹—SO₂N(R¹³)₂ group, wherein R¹¹, R¹² and R¹³ are as previously defined. Typically, the ortho-fused 5- or 6-membered cyclic group is non-aromatic. For example, the ortho-fused 5- or 6-membered cyclic group may be an ortho-fused 5- or 6-membered cycloalkyl group or an ortho-fused non-aromatic 5- or 6-membered heterocyclic group. In one embodiment, the ortho-fused 5- or 6-membered cyclic group is unsubstituted or substituted with one or more halo groups and/or one, two or three substituents independently selected from a C₁-C₆ alkyl, C₁-C₆ haloalkyl, C₂-C₆ alkenyl, C₂-C₆ haloalkenyl, —R¹⁵-R¹⁶, —R¹⁵—CN, —R¹⁵—N(R¹⁷)₂, —R¹⁵—OR¹⁷, —R¹⁵—COR¹⁷, —R¹⁵—COOR¹⁷, —R¹⁵—CON(R¹⁷)₂, —R¹⁵—C(═NR¹⁷)R¹⁷, —R¹⁵—C(═NR¹⁷)N(R¹⁷)₂, —R¹⁵—C(═NOR¹⁷)R¹⁷, —R¹⁵—SO₂R¹⁷ or —R¹⁵—SO₂N(R¹⁷)₂ group, wherein R¹⁵, R¹⁶ and R¹⁷ are as previously defined. In another embodiment, the ortho-fused 5- or 6-membered cyclic group is unsubstituted or substituted with one or more halo groups and/or one, two or three substituents independently selected from a —OH, —CN, —NO₂, C₁-C₄ alkyl, C₁-C₄ haloalkyl, —O(C₁-C₄ alkyl) or —O(C₁-C₄ haloalkyl) group. Typically, the ortho-fused 5- or 6-membered cyclic group is unsubstituted or substituted with one or more halo groups and/or one, two or three substituents independently selected from a —OH, —CN, C₁-C₄ alkyl, C₁-C₄ haloalkyl, —O(C₁-C₄ alkyl) or —O(C₁-C₄ haloalkyl) group. More typically, the ortho-fused 5- or 6-membered cyclic group is unsubstituted or substituted with one or more halo groups.

As will be understood, in accordance with either of the above two aspects of the second exemplary embodiment, the divalent phenyl or 5- or 6-membered heteroaryl group of L⁴ may optionally be further substituted with one or more halo groups and/or one or more further substituents R^{L}. Typically, the divalent phenyl or 5- or 6-membered heteroaryl group of L⁴ may optionally be further substituted with one or more halo groups and/or one or two substituents each independently selected from a —CN, methyl, halomethyl, —OC(R¹⁹)₃ or —C(R¹⁹)₂—OC(R¹⁹)₃ group, wherein each R¹⁹ is independently selected from hydrogen or a halo group. More typically, the divalent phenyl or 5- or 6-membered heteroaryl group of L⁴ may optionally be further substituted with one or more halo groups and/or one or two substituents each independently selected from a —CN, methyl, halomethyl, —OMe or —O-(halomethyl) group. More typically still, the divalent phenyl or 5- or 6-membered heteroaryl group of L⁴ may optionally be further substituted with one or more halo groups and/or one or two methyl and/or halomethyl substituents.

In one aspect of either the first or second exemplary embodiment, where L¹ is a divalent phenyl or 5- or 6-membered heteroaryl group, the ring atom of L¹ that is directly attached to L² is at the α- or β-position relative to the ring atom of L¹ that is directly attached to the sulfur atom of J. In a further aspect of either the first or second exemplary embodiment, where L¹ is a divalent phenyl or 5- or 6-membered heteroaryl group, the ring atom of L¹ that is directly attached to L² is at the β-position relative to the ring atom of L¹ that is directly attached to the sulfur atom of J.

In another aspect of either the first or second exemplary embodiment, where L³ is a divalent phenyl, or 5- or 6-membered heteroaryl group, it is unsubstituted or substituted with one or more halo groups and/or one or two substituents R^{L}. In one aspect, where L³ is a divalent phenyl or 5- or 6-membered heteroaryl group, the ring atom of L³ that is directly attached to L² is at the α- or β-position relative to the ring atom of L³ that is directly attached to L⁴. In a further aspect of either the first or second exemplary embodiment, where L³ is a divalent phenyl or 5- or 6-membered heteroaryl group, the ring atom of L³ that is directly attached to L² is at the β-position relative to the ring atom of L³ that is directly attached to L⁴.

In yet another aspect of either the first or second exemplary embodiment, L³ is a divalent phenyl or 6-membered heteroaryl group, wherein the divalent phenyl or 6-membered heteroaryl group may optionally be substituted with one or more halo groups and/or one or more substituents R^{L}. Typically in such an aspect, the divalent phenyl or 6-membered heteroaryl group of L³ is unsubstituted or substituted with one or more halo groups and/or one or two substituents R^{L}. Typically in such an aspect, the ring atom of L³ that is directly attached to L² is at the α- or β-position relative to the ring atom of L³ that is directly attached to L⁴. More typically in such an aspect, the ring atom of L³ that is directly attached to L² is at the β-position relative to the ring atom of L³ that is directly attached to L⁴.

In a third exemplary embodiment, where the compound has the formula (Ib):
L¹ is a bond, a divalent 3- to 7-membered monocyclic group, or a divalent 7- to 11-membered bicyclic group, wherein the divalent 3- to 7-membered monocyclic group or divalent 7- to 11-membered bicyclic group may optionally be substituted with one or more halo groups and/or one or more oxo (═O) groups and/or one or more substituents R^{L};
L² is an alkylene or alkenylene group, wherein the alkylene or alkenylene group may be straight-chained or branched, or be or include one or more cyclic groups, wherein one or more carbon atoms in the backbone of the alkylene or alkenylene group may optionally be replaced by one or more heteroatoms independently selected from N and O, and wherein the alkylene or alkenylene group may optionally be substituted with one or more halo groups;
L³ is a bond;
L⁴ is a phenyl or 5- or 6-membered heteroaryl group, wherein a ring atom of the phenyl or 5- or 6-membered heteroaryl group is directly attached to X, wherein a first 5- or 6-membered cyclic group is fused to the phenyl or 5- or 6-membered heteroaryl group across the α,β positions of the phenyl or 5- or 6-membered heteroaryl group, relative to the ring atom that is directly attached to X, wherein a ring atom of the first fused 5- or 6-membered cyclic group is directly attached to L², wherein optionally a second 5- or 6-membered cyclic group is fused to the phenyl or 5- or 6-membered heteroaryl group, wherein the phenyl or 5- or 6-membered heteroaryl group may optionally be further substituted with one or more halo groups and/or one or more substituents R^{L}, and wherein either fused 5- or 6-membered cyclic group may optionally be substituted with one or more halo groups and/or one or more oxo (═O) groups and/or one or more substituents R^{L};
each R^{L} is independently selected from a C₁-C₆ alkyl, C₁-C₆ haloalkyl, C₂-C₆ alkenyl, C₂-C₆ haloalkenyl, —R¹¹-R¹², —R¹¹—CN, —R¹¹—N₃, —R¹¹—NO₂, —R¹¹—N(R¹³)₂, —R¹¹—OR¹³, —R¹¹—COR¹³, —R¹¹—COOR¹³, —R¹¹—CON(R¹³)₂, —R¹¹—C(═NR¹³)R¹³, —R¹¹—C(═NR¹³)N(R¹³)₂, —R¹¹—C(═NOR¹³)R¹³, —R¹¹—SO₂R¹³ or —R¹¹—SO₂N(R¹³)₂ group; and
R¹¹, R¹² and R¹³ are as previously defined.

Typically in accordance with the third exemplary embodiment, each R^{L} is independently selected from a C₁-C₆ alkyl, C₁-C₆ haloalkyl, C₂-C₆ alkenyl, C₂-C₆ haloalkenyl, —R¹⁵-R¹⁶, —R¹⁵—CN, —R¹⁵—N(R¹⁷)₂, —R¹⁵—OR¹⁷, —R¹⁵—COR¹⁷, —R¹⁵—COOR¹⁷, —R¹⁵—CON(R¹⁷)₂, —R¹⁵—C(═NR¹⁷)R¹⁷, —R¹⁵—C(═NR¹⁷)N(R¹⁷)₂, —R¹⁵—C(═NOR¹⁷)R¹⁷, —R¹⁵—SO₂R¹⁷ or —R¹⁵—SO₂N(R¹⁷)₂ group, wherein R¹⁵, R¹⁶ and R¹⁷ are as previously defined.

In one aspect of the third exemplary embodiment, L¹ is a bond.

In another aspect of the third exemplary embodiment, L¹ is a divalent 3- to 7-membered monocyclic group, or a divalent 7- to 11-membered bicyclic group, wherein the divalent 3- to 7-membered monocyclic group or divalent 7- to 11-membered bicyclic group may optionally be substituted with one or more halo groups and/or one or more oxo (═O) groups and/or one or more substituents R^{L}.

In one aspect of the third exemplary embodiment, L¹ is a divalent phenyl, or 5- or 6-membered heteroaryl group, wherein the divalent phenyl or 5- or 6-membered heteroaryl group may optionally be substituted with one or more halo groups and/or one or more substituents R^{L}. Typically, where L¹ is a divalent phenyl, or 5- or 6-membered heteroaryl group, it is unsubstituted or substituted with one or more halo groups and/or one or two substituents R^{L}.

In another aspect of the third exemplary embodiment, L¹ is a divalent phenyl, or 5- or 6-membered heteroaryl group, wherein the divalent phenyl or 5- or 6-membered heteroaryl group is ortho-fused to a 5- or 6-membered cyclic group, wherein the divalent phenyl or 5- or 6-membered heteroaryl group may optionally be further substituted with one or two substituents independently selected from halo groups and R^{L}, and wherein the fused 5- or 6-membered cyclic group may optionally be substituted with one or more halo groups and/or one or more oxo (═O) groups and/or one or more substituents R^{L}. Typically, the divalent phenyl or 5- or 6-membered heteroaryl group is (aside from the fused 5- or 6-membered cyclic group) unsubstituted or further substituted with one or two halo groups and/or a single substituent R^{L}. Typically, the fused 5- or 6-membered cyclic group is unsubstituted or substituted with one or more halo groups and/or one or two substituents independently selected from oxo (═O) and R^{L}. In one embodiment, the fused 5- or 6-membered cyclic group is non-aromatic, such as a fused non-aromatic 5- or 6-membered heterocyclic group. In another embodiment, the fused 5- or 6-membered cyclic group is aromatic, such as a fused 5- or 6-membered heteroaryl group.

Typically, in either of the above two aspects of the third exemplary embodiment, where L¹ is a divalent phenyl or 5- or 6-membered heteroaryl group, the ring atom of L¹ that is directly attached to L² is at the α- or β-position relative to the ring atom of L¹ that is directly attached to the sulfur atom of J. More typically, where L¹ is a divalent phenyl or 5- or 6-membered heteroaryl group, the ring atom of L¹ that is directly attached to L² is at the β-position relative to the ring atom of L¹ that is directly attached to the sulfur atom of J.

In another aspect of the third exemplary embodiment, L¹ is a divalent saturated 4- to 7-membered monocyclic heterocyclic group, wherein the divalent saturated 4- to 7-membered monocyclic heterocyclic group may optionally be substituted with one or more halo groups and/or one or more oxo (═O) groups and/or one or more substituents R^{L}. For example, L¹ may be a divalent saturated 4- to 7-membered monocyclic heterocyclic group, wherein the divalent saturated 4- to 7-membered monocyclic heterocyclic group includes one or two heteroatoms independently selected from nitrogen and oxygen in its ring structure, and wherein the divalent saturated 4- to 7-membered monocyclic heterocyclic group may optionally be substituted with one or more halo groups and/or one or more oxo (═O) groups and/or one or more substituents R^{L}. Typically, the divalent saturated 4- to 7-membered monocyclic heterocyclic group includes at least one nitrogen atom in its ring structure. For example, L¹ may be selected from a divalent azetidinyl, pyrrolidinyl, piperidinyl, piperazinyl or morpholinyl group, any of which may optionally be substituted with one or more halo groups and/or one or more oxo (═O) groups and/or one or more substituents R^{L}. Typically, where L¹ is a divalent saturated 4- to 7-membered monocyclic heterocyclic group, such as a divalent azetidinyl, pyrrolidinyl, piperidinyl, piperazinyl or morpholinyl group, it is unsubstituted or substituted with one or more halo groups and/or one or two oxo (═O) groups and/or one or two substituents R^{L}. In one embodiment, where the divalent saturated 4- to 7-membered monocyclic heterocyclic group includes at least one nitrogen atom in its ring structure, the ring atom of L¹ that is directly attached to the sulfur atom of J is a nitrogen atom. Typically, where L¹ is a divalent saturated 4- to 7-membered monocyclic heterocyclic group, the ring atom of L¹ that is directly attached to L² is at the α-, β- or γ-position relative to the ring atom of L¹ that is directly attached to the sulfur atom of J. In one embodiment, where L¹ is a divalent saturated 4- to 7-membered monocyclic heterocyclic group, the ring atom of L¹ that is directly attached to L² is at the β-position relative to the ring atom of L¹ that is directly attached to the sulfur atom of J.

Typically in accordance with the third exemplary embodiment, L² contains in total from 2 to 15 carbon, nitrogen and oxygen atoms. More typically, L² contains in total from 2 to 10 carbon, nitrogen and oxygen atoms. Typically, L² contains in total from 0 to 3 nitrogen and oxygen atoms.

Typically in accordance with the third exemplary embodiment, L² has a chain length of from 2 to 12 atoms. More typically, L² has a chain length of from 2 to 8 atoms. In one aspect of the third exemplary embodiment, L² is an alkylene or alkenylene group, wherein the alkylene or alkenylene group may be straight-chained or branched, or be or include one or more cyclic groups, and wherein the alkylene or alkenylene group may optionally be substituted with one or more halo groups and/or one or two oxo (═O) groups. More typically in such an aspect, L² is an alkylene group, wherein the alkylene group may be straight-chained or branched, or include a single monocyclic group, wherein the alkylene group may optionally be substituted with one or more halo groups, and wherein L² contains in total from 2 to 15 carbon atoms. More typically still, L² is an alkylene group, wherein the alkylene group may be straight-chained or branched, wherein the alkylene group may optionally be substituted with one or more halo groups, and wherein L² contains in total from 2 to 15 carbon atoms.

Typically, in accordance with the third exemplary embodiment, the first fused 5- or 6-membered cyclic group of L⁴ and, if present, the second fused 5- or 6-membered cyclic group of L⁴ are non-aromatic. For example, the first and the second fused 5- or 6-membered cyclic groups may each be independently selected from an ortho-fused 5- or 6-membered cycloalkyl group or an ortho-fused non-aromatic 5- or 6-membered heterocyclic group.

In one aspect of the third exemplary embodiment, L⁴ is a phenyl or 5- or 6-membered heteroaryl group, wherein a ring atom of the phenyl or 5- or 6-membered heteroaryl group is directly attached to X, wherein a 5- or 6-membered cyclic group is fused to the phenyl or 5- or 6-membered heteroaryl group across the α,β positions of the phenyl or 5- or 6-membered heteroaryl group, relative to the ring atom that is directly attached to X, wherein a ring atom of the fused 5- or 6-membered cyclic group is directly attached to L², wherein the phenyl or 5- or 6-membered heteroaryl group of L⁴ is substituted at the α′-position with a substituent R^{L}, wherein the phenyl or 5- or 6-membered heteroaryl group may optionally be further substituted with one or two halo groups and/or one or two further substituents R^{L}, and wherein the fused 5- or 6-membered cyclic group may optionally be substituted with one or more halo groups and/or one or more oxo (═O) groups and/or one or more substituents R^{L}. Typically, the substituent at the α′-position is selected from a C₁-C₆ alkyl, C₁-C₆ haloalkyl, C₂-C₆ alkenyl, C₂-C₆ haloalkenyl, —R¹⁵-R¹⁶, —R¹⁵—CN, —R¹⁵—N(R¹⁷)₂, —R¹⁵—OR¹⁷, —R¹⁵—COR¹⁷, —R¹⁵—COOR¹⁷ or —R¹⁵—CON(R¹⁷)₂ group, wherein R¹⁵, R¹⁶ and R¹⁷ are as previously defined. More typically, the substituent at the α′-position is selected from a C₁-C₆ alkyl, C₁-C₆ haloalkyl, C₂-C₆ alkenyl, C₂-C₆ haloalkenyl, or 3- to 6-membered cyclic group, wherein the 3- to 6-membered cyclic group may optionally be substituted with one or more halo groups. Typically, where the phenyl or 5- or 6-membered heteroaryl group is further substituted with one or two halo groups and/or one or two further substituents R^{L}, the phenyl or 5- or 6-membered heteroaryl group is further substituted with one or two substituents each independently selected from a halo, —CN, methyl, halomethyl, —OC(R¹⁹)₃ or —C(R¹⁹)₂—OC(R¹⁹)₃ group, wherein each R¹⁹ is independently selected from hydrogen or a halo group. More typically where the phenyl or 5- or 6-membered heteroaryl group is further substituted with one or two halo groups and/or one or two further substituents R^{L}, the phenyl or 5- or 6-membered heteroaryl group is further substituted with one or two substituents each independently selected from a halo, —CN, methyl, halomethyl, —OMe or —O-(halomethyl) group.

In another aspect of the third exemplary embodiment, L⁴ is a phenyl or 5- or 6-membered heteroaryl group, wherein a ring atom of the phenyl or 5- or 6-membered heteroaryl group is directly attached to X, wherein a first 5- or 6-membered cyclic group is fused to the phenyl or 5- or 6-membered heteroaryl group across the α,β positions of the phenyl or 5- or 6-membered heteroaryl group, relative to the ring atom that is directly attached to X, wherein a ring atom of the first fused 5- or 6-membered cyclic group is directly attached to L², wherein a second 5- or 6-membered cyclic group is fused to the phenyl or 5- or 6-membered heteroaryl group across the α′,β′-positions of the phenyl or 5- or 6-membered heteroaryl group, wherein the phenyl group of L⁴ may optionally be further substituted with a halo group or a substituent R^{L}, and wherein either fused 5- or 6-membered cyclic group may optionally be substituted with one or more halo groups and/or one or more oxo (═O) groups and/or one or more substituents R^{L}. Typically, where the phenyl group of L⁴ is further substituted with a halo group or a substituent R^{L}, the phenyl group is further substituted with a halo, —CN, methyl, halomethyl, —OC(R¹⁹)₃ or —C(R¹⁹)₂—OC(R¹⁹)₃ group, wherein each R¹⁹ is independently selected from hydrogen or a halo group. More typically where the phenyl group of L⁴ is further substituted with a halo group or a substituent R^{L}, the phenyl group is further substituted with a halo, —CN, methyl, halomethyl, —OMe or —O-(halomethyl) group.

Typically, in either of the above two aspects of the third exemplary embodiment, any 5- or 6-membered cyclic group that is fused to the phenyl or 5- or 6-membered heteroaryl group of L⁴ is unsubstituted or is substituted with one or more halo groups and/or one or two oxo (═O) groups and/or one, two or three substituents independently selected from a C₁-C₆ alkyl, C₁-C₆ haloalkyl, C₂-C₆ alkenyl, C₂-C₆ haloalkenyl, —R¹¹-R¹², —R¹¹—CN, —R¹¹—N₃, —R¹¹—NO₂, —R¹¹—N(R¹³)₂, —R¹¹—OR¹³, —R¹¹—COR¹³, —R¹¹—COOR¹³, —R¹¹—CON(R¹³)₂, —R¹¹—C(═NR¹³)R¹³, —R¹¹—C(═NR¹³)N(R¹³)₂, —R¹¹—C(═NOR¹³)R¹³, —R¹¹—SO₂R¹³ or —R¹¹—SO₂N(R¹³)₂ group, wherein R¹¹, R¹² and R¹³ are as previously defined. Typically, any such fused 5- or 6-membered cyclic group is unsubstituted or is substituted with one or more halo groups and/or one oxo (═O) group and/or one, two or three substituents independently selected from a C₁-C₆ alkyl, C₁-C₆ haloalkyl, C₂-C₆ alkenyl, C₂-C₆ haloalkenyl, —R¹⁵-R¹⁶, —R¹⁵—CN, —R¹⁵—N(R¹⁷)₂, —R¹⁵—OR¹⁷, —R¹⁵—COR¹⁷, —R¹⁵—COOR¹⁷, —R¹⁵—CON(R¹⁷)₂, —R¹⁵—C(═NR¹⁷)R¹⁷, —R¹⁵—C(═NR¹⁷)N(R¹⁷)₂, —R¹⁵—C(═NOR¹⁷)R¹⁷, —R¹⁵—SO₂R¹⁷ or —R¹⁵—SO₂N(R¹⁷)₂ group, wherein R¹⁵, R¹⁶ and R¹⁷ are as previously defined. More typically, any such fused 5- or 6-membered cyclic group is unsubstituted or is substituted with one or more halo groups and/or one oxo (═O) group and/or one, two or three substituents independently selected from a —OH, —CN, C₁-C₄ alkyl, C₁-C₄ haloalkyl, —O(C₁-C₄ alkyl) or —O(C₁-C₄ haloalkyl) group. More typically still, any such fused 5- or 6-membered cyclic group is unsubstituted or substituted with one or more halo groups.

Typically, in accordance with the third exemplary embodiment, the ring atom of the (first) fused 5- or 6-membered cyclic group of L⁴ that is directly attached to L² is also directly attached to the ring atom at the α-position of the phenyl or 5- or 6-membered heteroaryl group of L⁴.

In a fourth exemplary embodiment of the first aspect of the invention, the compound has the formula (Ic):

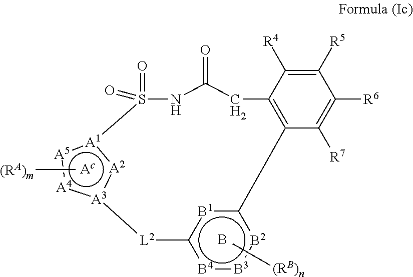

wherein:
A¹ and A³ are each independently selected from C and N, and A², A⁴ and A⁵ are each independently selected from N, C—H, C-Hal and N—H, such that ring A^{c} is a 5-membered heteroaryl ring containing one, two or three nitrogen atoms in its ring structure;
B¹, B², B³ and B⁴ are each independently selected from N, C—H and C-Hal, such that ring B is a 6-membered aryl ring or a 6-membered heteroaryl ring containing one, two or three nitrogen atoms in its ring structure;
m is 0, 1 or 2;
n is 0, 1 or 2;
each R^{A} is independently selected from —OH, —NH₂, —CN or a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{A} contains, in total, from 1 to 10 carbon, nitrogen and oxygen atoms, or wherein any two R^{A} attached to A⁴ and A⁵ may together form a fused 5- or 6-membered cyclic group, wherein the fused 5- or 6-membered cyclic group may optionally be substituted with one or more Hal groups and/or one or two groups independently selected from oxo (═O) and R^{AA};
each R^{AA} is independently selected from —OH, —NH₂, —CN or a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{AA} contains, in total, from 1 to 10 carbon, nitrogen and oxygen atoms;
each R^{B} is independently selected from a —CN, —NO₂, —R^{B1}, —OH, —OR^{B1}, —NH₂, —NHR^{B1} or —N(R^{B1})₂ group, wherein each R^{B1} is independently selected from a C₁-C₄ alkyl or C₁-C₄ fluoroalkyl group;
each Hal is independently selected from F, Cl or Br;
L² is a straight-chained alkylene or alkenylene group, wherein the straight-chained alkylene or alkenylene group optionally includes one or two heteroatoms independently selected from O and N in its carbon skeleton, wherein L² has a chain length of from 2 to 8 atoms, and wherein L² may optionally be substituted with one or two oxo (═O) groups and/or with one or more groups R^{L2}, wherein each R^{L2} is independently selected from a fluoro, C₁-C₄ alkyl, —O—(C₁-C₄ alkyl), C₁-C₄ fluoroalkyl or —O—(C₁-C₄ fluoroalkyl) group, or wherein any two R^{L2} may together with the atom(s) of the alkylene or alkenylene group to which they are attached form a 3- to 7-membered cyclic group, wherein the 3- to 7-membered cyclic group may optionally be substituted with one or more Hal groups and/or one or two oxo (═O) groups;
R⁴ is selected from a C₁-C₄ alkyl, C₁-C₄ fluoroalkyl, C₃-C₆ cycloalkyl or C₃-C₆ fluorocycloalkyl group, and R⁵ is selected from hydrogen, F, Cl, Br, or a —CN, methyl, fluoromethyl, —OC(R²⁰)₃ or —C(R²⁰)₂—OC(R²⁰)₃ group, or R⁴ and R⁵ together form a divalent group selected from —CH₂CH₂CH₂—, —CH═CHCH₂—, —CH₂CH═CH—, —CH₂CH₂O— and —OCH₂CH₂—, wherein the divalent group formed by R⁴ and R⁵ may optionally be fluoro-substituted;
R⁶ and R⁷ are each independently selected from hydrogen, F, Cl, Br, or a —CN, methyl, fluoromethyl, —OC(R²⁰)₃ or —C(R²⁰)₂—OC(R²⁰)₃ group; and
each R²⁰ is independently selected from hydrogen or F.

In one aspect of the fourth exemplary embodiment:
each R^{A} is independently selected from —OH, —NH₂, —CN or a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{A} contains, in total, from 1 to 6 carbon, nitrogen and oxygen atoms, or wherein any two R^{A} attached to A⁴ and A⁵ may together form a fused 5- or 6-membered cyclic group, wherein the fused 5- or 6-membered cyclic group may optionally be substituted with one or more Hal groups and/or one or two groups independently selected from oxo (═O) and R^{AA};
each R^{AA} is independently selected from —OH, —NH₂, —CN or a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{AA} contains, in total, from 1 to 6 carbon, nitrogen and oxygen atoms;
R⁴ is selected from a C₁-C₄ alkyl, C₁-C₄ fluoroalkyl, C₃-C₆ cycloalkyl or C₃-C₆ fluorocycloalkyl group, and R⁵ is selected from hydrogen, F, Cl, Br, or a —CN, methyl, fluoromethyl, —OMe or —O-(fluoromethyl) group, or R⁴ and R⁵ together form a divalent group selected from —CH₂CH₂CH₂—, —CH═CHCH₂—, —CH₂CH═CH—, —CH₂CH₂O— and —OCH₂CH₂—, wherein the divalent group formed by R⁴ and R⁵ may optionally be fluoro-substituted; and
R⁶ and R⁷ are each independently selected from hydrogen, F, Cl, Br, or a —CN, methyl, fluoromethyl, —OMe or —O-(fluoromethyl) group.

In another aspect of the fourth exemplary embodiment:
each R^{A} is independently selected from a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally fluoro-substituted, and wherein each R^{A} contains, in total, from 1 to 6 carbon, nitrogen and oxygen atoms;
L² is a straight-chained alkylene group, wherein the straight-chained alkylene group optionally includes one or two heteroatoms independently selected from O and N in its carbon skeleton, wherein L² has a chain length of from 2 to 8 atoms, and wherein L² may optionally be substituted with one or two oxo (═O) groups and/or with one or more groups R^{L2}, wherein each R^{L2} is independently selected from a fluoro, methyl or fluoromethyl group, or wherein any two R^{L2} attached to the same carbon atom may together with the carbon atom to which they are attached form a cyclopropyl group, wherein the cyclopropyl group may optionally be fluoro-substituted;
R⁴ is selected from a C₁-C₄ alkyl, C₁-C₄ fluoroalkyl, C₃-C₆ cycloalkyl or C₃-C₆ fluorocycloalkyl group, and R⁵ is selected from hydrogen, F, Cl, Br or a methyl or fluoromethyl group, or R⁴ and R⁵ together form a divalent group selected from —CH₂CH₂CH₂—, —CH═CHCH₂—, —CH₂CH═CH—, —CH₂CH₂O— and —OCH₂CH₂—, wherein the divalent group formed by R⁴ and R⁵ may optionally be fluoro-substituted; and
R⁶ and R⁷ are each independently selected from hydrogen, F, Cl, Br or a methyl or fluoromethyl group.

For the purposes of the present specification, where it is stated that A², A⁴ or A⁵ may be N—H or C—H, it is to be understood that this refers to A², A⁴ and A⁵ before possible substitution with R^{A} is considered. Thus, where it is stated that A², A⁴ or A⁵ may be N—H, it is to be understood that A², A⁴ or A⁵ may be N—H or N—R^{A} after substitution is considered. Similarly, where it is stated that A², A⁴ or A⁵ may be C—H, it is to be understood that A², A⁴ or A⁵ may be C—H or C—R^{A} after substitution is considered.

Likewise, where it is stated that B¹, B², B³ or B⁴ may be C—H, it is to be understood that this refers to B¹, B², B³ and B⁴ before possible substitution with R^{B} is considered. Thus, where it is stated that B¹, B², B³ or B⁴ may be C—H, it is to be understood that B¹, B², B³ or B⁴ may be C—H or C—R^{B} after substitution is considered.

In one aspect of the fourth exemplary embodiment, ring A^{c} is a 5-membered heteroaryl ring containing two or three nitrogen atoms in its ring structure.

In another aspect of the fourth exemplary embodiment, A¹ is C, A³ is independently selected from C and N, and A², A⁴ and A⁵ are each independently selected from N, C—H, C-Hal and N—H, such that ring A^{c} is a 5-membered heteroaryl ring containing two or three nitrogen atoms in its ring structure. Typically in such an aspect, ring A^{c} is a 5-membered heteroaryl ring containing two nitrogen atoms in its ring structure. In one embodiment of such an aspect, ring A^{c} is a pyrazole ring.

In one aspect of the fourth exemplary embodiment:

each R^{A} is independently selected from a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{A} contains, in total, from 1 to 10 carbon, nitrogen and oxygen atoms, or wherein any two R^{A} attached to A⁴ and A⁵ may together form a fused 5- or 6-membered cyclic group, wherein the fused 5- or 6-membered cyclic group may optionally be substituted with one or more Hal groups and/or one or two groups independently selected from oxo (═O) and R^{AA}; and

each R^{AA} is independently selected from a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{AA} contains, in total, from 1 to 10 carbon, nitrogen and oxygen atoms.

In a further aspect of the fourth exemplary embodiment:
each R^{A} is independently selected from a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{A} contains, in total, from 1 to 6 carbon, nitrogen and oxygen atoms, or wherein any two R^{A} attached to A⁴ and A⁵ may together form a fused 5- or 6-membered cyclic group, wherein the fused 5- or 6-membered cyclic group may optionally be substituted with one or more Hal groups and/or one or two groups independently selected from oxo (═O) and R^{AA}; and
each R^{AA} is independently selected from a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{AA} contains, in total, from 1 to 6 carbon, nitrogen and oxygen atoms.

In one aspect of the fourth exemplary embodiment, m is 0 or 1.

In another aspect of the fourth exemplary embodiment, m is 0.

In one aspect of the fourth exemplary embodiment, each R^{A} is independently selected from —OH, —NH₂, —CN or a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{A} contains, in total, from 1 to 10 carbon, nitrogen and oxygen atoms. Typically in such an aspect, each R^{A} contains, in total, from 1 to 6 carbon, nitrogen and oxygen atoms. Typically in such an aspect, each R^{A} is independently selected from a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{A} contains, in total, from 1 to 10 (or, more typically, from 1 to 6) carbon, nitrogen and oxygen atoms.

In another aspect of the fourth exemplary embodiment, each R^{A} is independently selected from a saturated hydrocarbyl group, wherein the saturated hydrocarbyl group is straight-chained or branched, or is or includes a cyclic group, wherein the saturated hydrocarbyl group optionally includes a single heteroatom O or N in its carbon skeleton, wherein the saturated hydrocarbyl group is optionally substituted with one or more fluoro groups and/or a single oxo (═O) group, and wherein each R^{A} contains, in total, from 1 to 5 carbon, nitrogen and oxygen atoms. In one embodiment of such an aspect, each R^{A} is independently selected from a saturated hydrocarbyl group, wherein the saturated hydrocarbyl group is straight-chained or branched, or is or includes a cyclic group, wherein the saturated hydrocarbyl group optionally includes a single heteroatom O or N in its carbon skeleton, wherein the saturated hydrocarbyl group is optionally fluoro-substituted, and wherein each R^{A} contains, in total, from 1 to 4 carbon, nitrogen and oxygen atoms.

In one aspect of the fourth exemplary embodiment, two R^{A} attached to A4 and A5 together form a fused 5- or 6-membered cyclic group, wherein the fused 5- or 6-membered cyclic group may optionally be substituted with one or more Hal groups and/or one or two groups independently selected from oxo (═O) and R^{AA}. Typically, the fused 5- or 6-membered cyclic group is unsubstituted or is substituted with one or more fluoro groups and/or a single oxo (═O) group and/or a single R^{AA} group. In one embodiment of such an aspect, the fused 5- or 6-membered cyclic group is a fused phenyl or 5- or 6-membered heteroaryl group, such as a fused pyridinyl group. In another embodiment of such an aspect, the fused 5- or 6-membered cyclic group is a fused 5- or 6-membered cycloalkyl group or a fused non-aromatic 5- or 6-membered heterocyclic group, such as a fused piperidine group.

In one aspect of the fourth exemplary embodiment, each R^{AA} contains, in total, from 1 to 6 carbon, nitrogen and oxygen atoms.

In another aspect of the fourth exemplary embodiment, each R^{AA} is independently selected from a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{AA} contains, in total, from 1 to 1 (or, more typically, from 1 to 6) carbon, nitrogen and oxygen atoms.

In another aspect of the fourth exemplary embodiment, each R^{AA} is independently selected from a saturated hydrocarbyl group, wherein the saturated hydrocarbyl group is straight-chained or branched, or is or includes a cyclic group, wherein the saturated hydrocarbyl group optionally includes a single heteroatom O or N in its carbon skeleton, wherein the saturated hydrocarbyl group is optionally substituted with one or more fluoro groups and/or a single oxo (═O) group, and wherein each R^{AA} contains, in total, from 1 to 5 carbon, nitrogen and oxygen atoms. In one embodiment of such an aspect, each R^{AA} is independently selected from a saturated hydrocarbyl group, wherein the saturated hydrocarbyl group is straight-chained or branched, or is or includes a cyclic group, wherein the saturated hydrocarbyl group optionally includes a single heteroatom O or N in its carbon skeleton, wherein the saturated hydrocarbyl group is optionally fluoro-substituted, and wherein each R^{AA} contains, in total, from 1 to 4 carbon, nitrogen and oxygen atoms. In a further embodiment of such an aspect, each R^{AA} is independently selected from a methyl or ethyl group, wherein the methyl or ethyl group may optionally be fluoro-substituted.

In a fifth exemplary embodiment of the first aspect of the invention, the compound has the formula (Id):

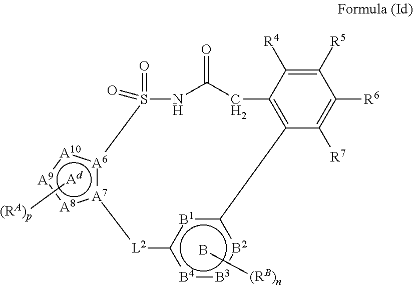

wherein:
A⁶ and A⁷ are each independently selected from C and N, and A⁸, A⁹ and A¹⁰ are each independently selected from N, C—H, C-Hal and N—H, such that ring A^{d} is a 5-membered heteroaryl ring containing one, two or three nitrogen atoms in its ring structure;
B¹, B², B³ and B⁴ are each independently selected from N, C—H and C-Hal, such that ring B is a 6-membered aryl ring or a 6-membered heteroaryl ring containing one, two or three nitrogen atoms in its ring structure;
p is 0, 1 or 2;
n is 0, 1 or 2;
each R^{A} is independently selected from —OH, —NH₂, —CN or a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{A} contains, in total, from 1 to 10 carbon, nitrogen and oxygen atoms, or wherein any two R^{A} attached to A⁸ and A⁹ or to A⁹ and A¹⁰ may together form a fused 5- or 6-membered cyclic group, wherein the fused 5- or 6-membered cyclic group may optionally be substituted with one or more Hal groups and/or one or two groups independently selected from oxo (═O) and R^{AA};
each R^{AA} is independently selected from —OH, —NH₂, —CN or a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{AA} contains, in total, from 1 to 10 carbon, nitrogen and oxygen atoms;
each R^{B} is independently selected from a —CN, —NO₂, —R^{B1}, —OH, —OR^{B1}, —NH₂, —NHR^{B1} or —N(R^{B1})₂ group, wherein each R^{B1} is independently selected from a C₁-C₄ alkyl or C₁-C₄ fluoroalkyl group;
each Hal is independently selected from F, Cl or Br;
L² is a straight-chained alkylene or alkenylene group, wherein the straight-chained alkylene or alkenylene group optionally includes one or two heteroatoms independently selected from O and N in its carbon skeleton, wherein L² has a chain length of from 2 to 8 atoms, and wherein L² may optionally be substituted with one or two oxo (═O) groups and/or with one or more groups R^{L2}, wherein each R^{L2} is independently selected from a fluoro, C₁-C₄ alkyl, —O—(C₁-C₄ alkyl), C₁-C₄ fluoroalkyl or —O—(C₁-C₄ fluoroalkyl) group, or wherein any two R^{L2} may together with the atom(s) of the alkylene or alkenylene group to which they are attached form a 3- to 7-membered cyclic group, wherein the 3- to 7-membered cyclic group may optionally be substituted with one or more Hal groups and/or one or two oxo (═O) groups;
R⁴ is selected from a C₁-C₄ alkyl, C₁-C₄ fluoroalkyl, C₃-C₆ cycloalkyl or C₃-C₆ fluorocycloalkyl group, and R⁵ is selected from hydrogen, F, Cl, Br, or a —CN, methyl, fluoromethyl, —OC(R²⁰)₃ or —C(R²⁰)₂—OC(R²⁰)₃ group, or R⁴ and R⁵ together form a divalent group selected from —CH₂CH₂CH₂—, —CH═CHCH₂—, —CH₂CH═CH—, —CH₂CH₂O— and —OCH₂CH₂—, wherein the divalent group formed by R⁴ and R⁵ may optionally be fluoro-substituted;
R⁶ and R⁷ are each independently selected from hydrogen, F, Cl, Br, or a —CN, methyl, fluoromethyl, —OC(R²⁰)₃ or —C(R²⁰)₂—OC(R²⁰)₃ group; and
each R²⁰ is independently selected from hydrogen or F.

In one aspect of the fifth exemplary embodiment:
each R^{A} is independently selected from —OH, —NH₂, —CN or a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{A} contains, in total, from 1 to 6 carbon, nitrogen and oxygen atoms, or wherein any two R^{A} attached to A⁸ and A⁹ or to A⁹ and A¹⁰ may together form a fused 5- or 6-membered cyclic group, wherein the fused 5- or 6-membered cyclic group may optionally be substituted with one or more Hal groups and/or one or two groups independently selected from oxo (═O) and R^{AA};
each R^{AA} is independently selected from —OH, —NH₂, —CN or a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{AA} contains, in total, from 1 to 6 carbon, nitrogen and oxygen atoms;
R⁴ is selected from a C₁-C₄ alkyl, C₁-C₄ fluoroalkyl, C₃-C₆ cycloalkyl or C₃-C₆ fluorocycloalkyl group, and R⁵ is selected from hydrogen, F, Cl, Br, or a —CN, methyl, fluoromethyl, —OMe or —O-(fluoromethyl) group, or R⁴ and R⁵ together form a divalent group selected from —CH₂CH₂CH₂—, —CH═CHCH₂—, —CH₂CH═CH—, —CH₂CH₂O— and —OCH₂CH₂—, wherein the divalent group formed by R⁴ and R⁵ may optionally be fluoro-substituted; and
R⁶ and R⁷ are each independently selected from hydrogen, F, Cl, Br, or a —CN, methyl, fluoromethyl, —OMe or —O-(fluoromethyl) group.

For the purposes of the present specification, where it is stated that A⁸, A⁹ or A¹⁰ may be N—H or C—H, it is to be understood that this refers to A⁸, A⁹ and A¹⁰ before possible substitution with R^{A} is considered. Thus, where it is stated that A⁸, A⁹ or A¹⁰ may be N—H, it is to be understood that A⁸, A⁹ or A¹⁰ may be N—H or N—R^{A} after substitution is considered. Similarly, where it is stated that A⁸, A⁹ or A¹⁰ may be C—H, it is to be understood that A⁸, A⁹ or A¹⁰ may be C—H or C—R^{A} after substitution is considered.

In one aspect of the fifth exemplary embodiment, ring A^{d} is a 5-membered heteroaryl ring containing two or three nitrogen atoms in its ring structure.

In another aspect of the fifth exemplary embodiment, A⁶ is C, A⁷ is independently selected from C and N, and A⁸, A⁹ and A¹⁰ are each independently selected from N, C—H, C-Hal and N—H, such that ring A^{d} is a 5-membered heteroaryl ring containing two or three nitrogen atoms in its ring structure. Typically in such an aspect, ring A^{d} is a 5-membered heteroaryl ring containing two nitrogen atoms in its ring structure. In one embodiment of such an aspect, ring A^{d} is a pyrazole ring.

In one aspect of the fifth exemplary embodiment:
each R^{A} is independently selected from a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{A} contains, in total, from 1 to 10 carbon, nitrogen and oxygen atoms, or wherein any two R^{A} attached to A⁸ and A⁹ or to A⁹ and A¹⁰ may together form a fused 5- or 6-membered cyclic group, wherein the fused 5- or 6-membered cyclic group may optionally be substituted with one or more Hal groups and/or one or two groups independently selected from oxo (═O) and R^{AA}; and
each R^{AA} is independently selected from a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{AA} contains, in total, from 1 to 10 carbon, nitrogen and oxygen atoms.

In a further aspect of the fifth exemplary embodiment:
each R^{A} is independently selected from a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{A} contains, in total, from 1 to 6 carbon, nitrogen and oxygen atoms, or wherein any two R^{A} attached to A⁸ and A⁹ or to A⁹ and A¹⁰ may together form a fused 5- or 6-membered cyclic group, wherein the fused 5- or 6-membered cyclic group may optionally be substituted with one or more Hal groups and/or one or two groups independently selected from oxo (═O) and R^{AA}; and
each R^{AA} is independently selected from a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{AA} contains, in total, from 1 to 6 carbon, nitrogen and oxygen atoms.

In one aspect of the fifth exemplary embodiment, p is 0 or 1.

In another aspect of the fifth exemplary embodiment, p is 0.

In one aspect of the fifth exemplary embodiment, each R^{A} is independently selected from —OH, —NH₂, —CN or a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{A} contains, in total, from 1 to 10 carbon, nitrogen and oxygen atoms. Typically in such an aspect, each R^{A} contains, in total, from 1 to 6 carbon, nitrogen and oxygen atoms. Typically in such an aspect, each R^{A} is independently selected from a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{A} contains, in total, from 1 to 10 (or, more typically, from 1 to 6) carbon, nitrogen and oxygen atoms.

In another aspect of the fifth exemplary embodiment, each R^{A} is independently selected from a saturated hydrocarbyl group, wherein the saturated hydrocarbyl group is straight-chained or branched, or is or includes a cyclic group, wherein the saturated hydrocarbyl group optionally includes a single heteroatom O or N in its carbon skeleton, wherein the saturated hydrocarbyl group is optionally substituted with one or more fluoro groups and/or a single oxo (═O) group, and wherein each R^{A} contains, in total, from 1 to 5 carbon, nitrogen and oxygen atoms. In one embodiment of such an aspect, each R^{A} is independently selected from a saturated hydrocarbyl group, wherein the saturated hydrocarbyl group is straight-chained or branched, or is or includes a cyclic group, wherein the saturated hydrocarbyl group optionally includes a single heteroatom O or N in its carbon skeleton, wherein the saturated hydrocarbyl group is optionally fluoro-substituted, and wherein each R^{A} contains, in total, from 1 to 4 carbon, nitrogen and oxygen atoms.

In a sixth exemplary embodiment of the first aspect of the invention, the compound has the formula (Ie):

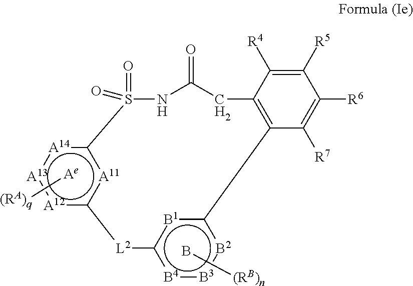

wherein:
A¹¹, A¹², A¹³ and A¹⁴ are each independently selected from N, C—H and C-Hal, such that ring A^{e} is a 6-membered aryl ring or a 6-membered heteroaryl ring containing one, two or three nitrogen atoms in its ring structure;
B¹, B², B³ and B⁴ are each independently selected from N, C—H and C-Hal, such that ring B is a 6-membered aryl ring or a 6-membered heteroaryl ring containing one, two or three nitrogen atoms in its ring structure;
q is 0, 1 or 2;
n is 0, 1 or 2;
each R^{A} is independently selected from —OH, —NH₂, —CN or a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{A} contains, in total, from 1 to 10 carbon, nitrogen and oxygen atoms, or wherein any two R^{A} attached to A¹² and A¹³ or to A¹³ and A¹⁴ may together form a fused 5- or 6-membered cyclic group, wherein the fused 5- or 6-membered cyclic group may optionally be substituted with one or more Hal groups and/or one or two groups independently selected from oxo (═O) and R^{AA};
each R^{AA} is independently selected from —OH, —NH₂, —CN or a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{AA} contains, in total, from 1 to 10 carbon, nitrogen and oxygen atoms;
each R^{B} is independently selected from a —CN, —NO₂, —R^{B1}, —OH, —OR^{B1}, —NH₂, —NHR^{B1} or —N(R^{B1})₂ group, wherein each R^{B1} is independently selected from a C₁-C₄ alkyl or C₁-C₄ fluoroalkyl group;
each Hal is independently selected from F, Cl or Br;
L² is a straight-chained alkylene or alkenylene group, wherein the straight-chained alkylene or alkenylene group optionally includes one or two heteroatoms independently selected from O and N in its carbon skeleton, wherein L² has a chain length of from 2 to 8 atoms, and wherein L² may optionally be substituted with one or two oxo (═O) groups and/or with one or more groups R^{L2}, wherein each R^{L2} is independently selected from a fluoro, C₁-C₄ alkyl, —O—(C₁-C₄ alkyl), C₁-C₄ fluoroalkyl or —O—(C₁-C₄ fluoroalkyl) group, or wherein any two R^{L2} may together with the atom(s) of the alkylene or alkenylene group to which they are attached form a 3- to 7-membered cyclic group, wherein the 3- to 7-membered cyclic group may optionally be substituted with one or more Hal groups and/or one or two oxo (═O) groups;
R⁴ is selected from a C₁-C₄ alkyl, C₁-C₄ fluoroalkyl, C₃-C₆ cycloalkyl or C₃-C₆ fluorocycloalkyl group, and R⁵ is selected from hydrogen, F, Cl, Br, or a —CN, methyl, fluoromethyl, —OC(R²⁰)₃ or —C(R²⁰)₂—OC(R²⁰)₃ group, or R⁴ and R⁵ together form a divalent group selected from —CH₂CH₂CH₂—, —CH═CHCH₂—, —CH₂CH═CH—, —CH₂CH₂O— and —OCH₂CH₂—, wherein the divalent group formed by R⁴ and R⁵ may optionally be fluoro-substituted;
R⁶ and R⁷ are each independently selected from hydrogen, F, Cl, Br, or a —CN, methyl, fluoromethyl, —OC(R²⁰)₃ or —C(R²⁰)₂—OC(R²⁰)₃ group; and
each R²⁰ is independently selected from hydrogen or F.

In one aspect of the sixth exemplary embodiment:
each R^{A} is independently selected from —OH, —NH₂, —CN or a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{A} contains, in total, from 1 to 6 carbon, nitrogen and oxygen atoms, or wherein any two R^{A} attached to A¹² and A¹³ or to A¹³ and A¹⁴ may together form a fused 5- or 6-membered cyclic group, wherein the fused 5- or 6-membered cyclic group may optionally be substituted with one or more Hal groups and/or one or two groups independently selected from oxo (═O) and R^{AA};
each R^{AA} is independently selected from —OH, —NH₂, —CN or a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{AA} contains, in total, from 1 to 6 carbon, nitrogen and oxygen atoms;
R⁴ is selected from a C₁-C₄ alkyl, C₁-C₄ fluoroalkyl, C₃-C₆ cycloalkyl or C₃-C₆ fluorocycloalkyl group, and R⁵ is selected from hydrogen, F, Cl, Br, or a —CN, methyl, fluoromethyl, —OMe or —O-(fluoromethyl) group, or R⁴ and R⁵ together form a divalent group selected from —CH₂CH₂CH₂—, —CH═CHCH₂—, —CH₂CH═CH—, —CH₂CH₂O— and —OCH₂CH₂—, wherein the divalent group formed by R⁴ and R⁵ may optionally be fluoro-substituted; and
R⁶ and R⁷ are each independently selected from hydrogen, F, Cl, Br, or a —CN, methyl, fluoromethyl, —OMe or —O-(fluoromethyl) group.

For the purposes of the present specification, where it is stated that A¹¹, A¹², A¹³ or A¹⁴ may be C—H, it is to be understood that this refers to A¹¹, A¹², A¹³ and A¹⁴ before possible substitution with R^{A} is considered. Thus, where it is stated that A¹¹, A¹², A¹³ or A¹⁴ may be C—H, it is to be understood that A¹¹, A¹², A¹³ or A¹⁴ may be C—H or C—R^{A} after substitution is considered.

In one aspect of the sixth exemplary embodiment, ring A^{e} is a 6-membered aryl ring or a 6-membered heteroaryl ring containing one or two nitrogen atoms in its ring structure.

In a further aspect of the sixth exemplary embodiment, ring Ae is a 6-membered aryl ring or a 6-membered heteroaryl ring containing one nitrogen atom in its ring structure. As will be understood, in such an aspect ring Ae is a phenyl or a pyridinyl ring. In one embodiment, A¹¹, A¹², A¹³ and A¹⁴ are each independently selected from C—H and C-Hal, such that ring A^{e} is a 6-membered aryl ring.

In one aspect of the sixth exemplary embodiment, q is 0 or 1.

In another aspect of the sixth exemplary embodiment, q is 0.

In one aspect of the sixth exemplary embodiment, each R^{A} is independently selected from —OH, —NH₂, —CN or a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{A} contains, in total, from 1 to 10 carbon, nitrogen and oxygen atoms. Typically in such an aspect, each R^{A} contains, in total, from 1 to 6 carbon, nitrogen and oxygen atoms. Typically in such an aspect, each R^{A} is independently selected from a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{A} contains, in total, from 1 to 10 (or, more typically, from 1 to 6) carbon, nitrogen and oxygen atoms.

In another aspect of the sixth exemplary embodiment, each R^{A} is independently selected from a saturated hydrocarbyl group, wherein the saturated hydrocarbyl group is straight-chained or branched, or is or includes a cyclic group, wherein the saturated hydrocarbyl group optionally includes a single heteroatom O or N in its carbon skeleton, wherein the saturated hydrocarbyl group is optionally substituted with one or more fluoro groups and/or a single oxo (═O) group, and wherein each R^{A} contains, in total, from 1 to 5 carbon, nitrogen and oxygen atoms. In one embodiment of such an aspect, each R^{A} is independently selected from a saturated hydrocarbyl group, wherein the saturated hydrocarbyl group is straight-chained or branched, or is or includes a cyclic group, wherein the saturated hydrocarbyl group optionally includes a single heteroatom O or N in its carbon skeleton, wherein the saturated hydrocarbyl group is optionally fluoro-substituted, and wherein each R^{A} contains, in total, from 1 to 4 carbon, nitrogen and oxygen atoms.

In a seventh exemplary embodiment of the first aspect of the invention, the compound has the formula (If):

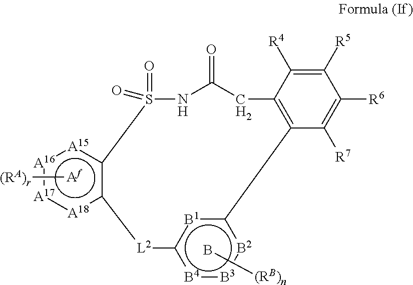

wherein:
A¹⁵, A¹⁶, A¹⁷ and A¹⁸ are each independently selected from N, C—H and C-Hal, such that ring A^{f} is a 6-membered aryl ring or a 6-membered heteroaryl ring containing one, two or three nitrogen atoms in its ring structure;
B¹, B², B³ and B⁴ are each independently selected from N, C—H and C-Hal, such that ring B is a 6-membered aryl ring or a 6-membered heteroaryl ring containing one, two or three nitrogen atoms in its ring structure;
r is 0, 1 or 2;
n is 0, 1 or 2;
each R^{A} is independently selected from —OH, —NH₂, —CN or a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{A} contains, in total, from 1 to 10 carbon, nitrogen and oxygen atoms, or wherein any two R^{A} attached to A¹⁵ and A¹⁶ or to A¹⁶ and A¹⁷ or to A¹⁷ and A¹⁸ may together form a fused 5- or 6-membered cyclic group, wherein the fused 5- or 6-membered cyclic group may optionally be substituted with one or more Hal groups and/or one or two groups independently selected from oxo (═O) and R^{AA};
each R^{AA} is independently selected from —OH, —NH₂, —CN or a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{AA} contains, in total, from 1 to 10 carbon, nitrogen and oxygen atoms;
each R^{B} is independently selected from a —CN, —NO₂, —R^{B1}, —OH, —OR^{B1}, —NH₂, —NHR^{B1} or —N(R^{B1})₂ group, wherein each R^{B1} is independently selected from a C₁-C₄ alkyl or C₁-C₄ fluoroalkyl group;
each Hal is independently selected from F, Cl or Br;
L² is a straight-chained alkylene or alkenylene group, wherein the straight-chained alkylene or alkenylene group optionally includes one or two heteroatoms independently selected from O and N in its carbon skeleton, wherein L² has a chain length of from 2 to 8 atoms, and wherein L² may optionally be substituted with one or two oxo (═O) groups and/or with one or more groups R^{L2}, wherein each R^{L2} is independently selected from a fluoro, C₁-C₄ alkyl, —O—(C₁-C₄ alkyl), C₁-C₄ fluoroalkyl or —O—(C₁-C₄ fluoroalkyl) group, or wherein any two R^{L2} may together with the atom(s) of the alkylene or alkenylene group to which they are attached form a 3- to 7-membered cyclic group, wherein the 3- to 7-membered cyclic group may optionally be substituted with one or more Hal groups and/or one or two oxo (═O) groups;
R⁴ is selected from a C₁-C₄ alkyl, C₁-C₄ fluoroalkyl, C₃-C₆ cycloalkyl or C₃-C₆ fluorocycloalkyl group, and R⁵ is selected from hydrogen, F, Cl, Br, or a —CN, methyl, fluoromethyl, —OC(R²⁰)₃ or —C(R²⁰)₂—OC(R²⁰)₃ group, or R⁴ and R⁵ together form a divalent group selected from —CH₂CH₂CH₂—, —CH═CHCH₂—, —CH₂CH═CH—, —CH₂CH₂O— and —OCH₂CH₂—, wherein the divalent group formed by R⁴ and R⁵ may optionally be fluoro-substituted;
R⁶ and R⁷ are each independently selected from hydrogen, F, Cl, Br, or a —CN, methyl, fluoromethyl, —OC(R²⁰)₃ or —C(R²⁰)₂—OC(R²⁰)₃ group; and
each R²⁰ is independently selected from hydrogen or F.

In one aspect of the seventh exemplary embodiment:
each R^{A} is independently selected from —OH, —NH₂, —CN or a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{A} contains, in total, from 1 to 6 carbon, nitrogen and oxygen atoms, or wherein any two R^{A} attached to A¹⁵ and A¹⁶ or to A¹⁶ and A¹⁷ or to A¹⁷ and A¹⁸ may together form a fused 5- or 6-membered cyclic group, wherein the fused 5- or 6-membered cyclic group may optionally be substituted with one or more Hal groups and/or one or two groups independently selected from oxo (═O) and R^{AA};
each R^{AA} is independently selected from —OH, —NH₂, —CN or a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{AA} contains, in total, from 1 to 6 carbon, nitrogen and oxygen atoms;
R⁴ is selected from a C₁-C₄ alkyl, C₁-C₄ fluoroalkyl, C₃-C₆ cycloalkyl or C₃-C₆ fluorocycloalkyl group, and R⁵ is selected from hydrogen, F, Cl, Br, or a —CN, methyl, fluoromethyl, —OMe or —O-(fluoromethyl) group, or R⁴ and R⁵ together form a divalent group selected from —CH₂CH₂CH₂—, —CH═CHCH₂—, —CH₂CH═CH—, —CH₂CH₂O— and —OCH₂CH₂—, wherein the divalent group formed by R⁴ and R⁵ may optionally be fluoro-substituted; and
R⁶ and R⁷ are each independently selected from hydrogen, F, Cl, Br, or a —CN, methyl, fluoromethyl, —OMe or —O-(fluoromethyl) group.

For the purposes of the present specification, where it is stated that A¹⁵, A¹⁶, A¹⁷ or A¹⁸ may be C—H, it is to be understood that this refers to A¹⁵, A¹⁶, A¹⁷ and A¹⁸ before possible substitution with R^{A} is considered. Thus, where it is stated that A¹⁵, A¹⁶, A¹⁷ or A¹⁸ may be C—H, it is to be understood that A¹⁵, A¹⁶, A¹⁷ or A¹⁸ may be C—H or C—R^{A} after substitution is considered.

In one aspect of the seventh exemplary embodiment, ring A^{f} is a 6-membered aryl ring or a 6-membered heteroaryl ring containing one or two nitrogen atoms in its ring structure.

In a further aspect of the seventh exemplary embodiment, ring A^{f} is a 6-membered aryl ring or a 6-membered heteroaryl ring containing one nitrogen atom in its ring structure. As will be understood, in such an aspect ring A^{f} is a phenyl or a pyridinyl ring. In one embodiment, A¹⁸ is N, and A¹⁵, A¹⁶ and A¹⁷ are each independently selected from C—H and C-Hal, such that ring A^{f} is a pyridinyl ring.

In one aspect of the seventh exemplary embodiment, r is 0 or 1.

In another aspect of the seventh exemplary embodiment, r is 0.

In one aspect of the seventh exemplary embodiment, each R^{A} is independently selected from —OH, —NH₂, —CN or a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{A} contains, in total, from 1 to 10 carbon, nitrogen and oxygen atoms. Typically in such an aspect, each R^{A} contains, in total, from 1 to 6 carbon, nitrogen and oxygen atoms. Typically in such an aspect, each R^{A} is independently selected from a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{A} contains, in total, from 1 to 10 (or, more typically, from 1 to 6) carbon, nitrogen and oxygen atoms.

In another aspect of the seventh exemplary embodiment, each R^{A} is independently selected from a saturated hydrocarbyl group, wherein the saturated hydrocarbyl group is straight-chained or branched, or is or includes a cyclic group, wherein the saturated hydrocarbyl group optionally includes a single heteroatom O or N in its carbon skeleton, wherein the saturated hydrocarbyl group is optionally substituted with one or more fluoro groups and/or a single oxo (═O) group, and wherein each R^{A} contains, in total, from 1 to 5 carbon, nitrogen and oxygen atoms. In one embodiment of such an aspect, each R^{A} is independently selected from a saturated hydrocarbyl group, wherein the saturated hydrocarbyl group is straight-chained or branched, or is or includes a cyclic group, wherein the saturated hydrocarbyl group optionally includes a single heteroatom O or N in its carbon skeleton, wherein the saturated hydrocarbyl group is optionally fluoro-substituted, and wherein each R^{A} contains, in total, from 1 to 4 carbon, nitrogen and oxygen atoms.

In an eighth exemplary embodiment of the first aspect of the invention, the compound has the formula (Ig):

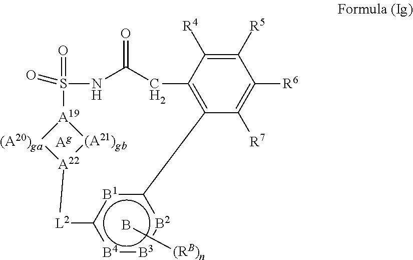

wherein:
A¹⁹ and A²² are each independently selected from N, CH, CY and CR^{AG}, and each A²⁰ and A²¹ is independently selected from O, NH, NR^{AGG}, C═O, CH₂, CH(Y), CH(R^{AG}), C(Y)₂, C(Y)(R^{AG}) and C(R^{AG})₂, such that ring A^{g} contains one or two atoms independently selected from oxygen and nitrogen in its ring structure;
ga is 1, 2 or 3, and gb is 1, 2 or 3, provided that ga+gb≤5;
each Y is independently selected from F, Cl or Br;
each R^{AG} is independently selected from —OH, —NH₂, —CN, or a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{AG} contains, in total, from 1 to 10 carbon, nitrogen and oxygen atoms;
each R^{AGG} is independently selected from a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{AGG} contains, in total, from 1 to 10 carbon, nitrogen and oxygen atoms;
B¹, B², B³ and B⁴ are each independently selected from N, C—H and C-Hal, such that ring B is a 6-membered aryl ring or a 6-membered heteroaryl ring containing one, two or three nitrogen atoms in its ring structure;
n is 0, 1 or 2;
each R^{B} is independently selected from a —CN, —NO₂, —R^{B1}, —OH, —OR^{B1}, —NH₂, —NHR^{B1} or —N(R^{B1})₂ group, wherein each R^{B1} is independently selected from a C₁-C₄ alkyl or C₁-C₄ fluoroalkyl group;
each Hal is independently selected from F, Cl or Br;
L² is a straight-chained alkylene or alkenylene group, wherein the straight-chained alkylene or alkenylene group optionally includes one or two heteroatoms independently selected from O and N in its carbon skeleton, wherein L² has a chain length of from 2 to 8 atoms, and wherein L² may optionally be substituted with one or two oxo (═O) groups and/or with one or more groups R^{L2}, wherein each R^{L2} is independently selected from a fluoro, C₁-C₄ alkyl, —O—(C₁-C₄ alkyl), C₁-C₄ fluoroalkyl or —O—(C₁-C₄ fluoroalkyl) group, or wherein any two R^{L2} may together with the atom(s) of the alkylene or alkenylene group to which they are attached form a 3- to 7-membered cyclic group, wherein the 3- to 7-membered cyclic group may optionally be substituted with one or more Hal groups and/or one or two oxo (═O) groups;
R⁴ is selected from a C₁-C₄ alkyl, C₁-C₄ fluoroalkyl, C₃-C₆ cycloalkyl or C₃-C₆ fluorocycloalkyl group, and R⁵ is selected from hydrogen, F, Cl, Br, or a —CN, methyl, fluoromethyl, —OC(R²⁰)₃ or —C(R²⁰)₂—OC(R²⁰)₃ group, or R⁴ and R⁵ together form a divalent group selected from —CH₂CH₂CH₂—, —CH═CHCH₂—, —CH₂CH═CH—, —CH₂CH₂O— and —OCH₂CH₂—, wherein the divalent group formed by R⁴ and R⁵ may optionally be fluoro-substituted;
R⁶ and R⁷ are each independently selected from hydrogen, F, Cl, Br, or a —CN, methyl, fluoromethyl, —OC(R²⁰)₃ or —C(R²⁰)₂—OC(R²⁰)₃ group; and
each R²⁰ is independently selected from hydrogen or F.

In one aspect of the eighth exemplary embodiment:
each R^{AG} is independently selected from —OH, —NH₂, —CN, or a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{AG} contains, in total, from 1 to 6 carbon, nitrogen and oxygen atoms;
each R^{AGG} is independently selected from a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{AGG} contains, in total, from 1 to 6 carbon, nitrogen and oxygen atoms;
R⁴ is selected from a C₁-C₄ alkyl, C₁-C₄ fluoroalkyl, C₃-C₆ cycloalkyl or C₃-C₆ fluorocycloalkyl group, and R⁵ is selected from hydrogen, F, Cl, Br, or a —CN, methyl, fluoromethyl, —OMe or —O-(fluoromethyl) group, or R⁴ and R⁵ together form a divalent group selected from —CH₂CH₂CH₂—, —CH═CHCH₂—, —CH₂CH═CH—, —CH₂CH₂O— and —OCH₂CH₂—, wherein the divalent group formed by R⁴ and R⁵ may optionally be fluoro-substituted; and
R⁶ and R⁷ are each independently selected from hydrogen, F, Cl, Br, or a —CN, methyl, fluoromethyl, —OMe or —O-(fluoromethyl) group.

As will be understood, since ring Ag contains one or two atoms independently selected from oxygen and nitrogen in its ring structure, all other atoms within the ring structure of ring A^{g} will be carbon atoms. Typically, each ring carbon atom of ring A^{g} is directly attached to at least one other ring carbon atom of ring Ag. Typically, each ring nitrogen or oxygen atom of ring Ag is directly attached to two ring carbon atoms of ring Ag.

Typically, in accordance with the eighth exemplary embodiment of the first aspect of the invention, each Y is F or Cl. More typically, each Y is F.

Typically, 3≤ga+gb≤4.

In one aspect of the eighth exemplary embodiment, A¹⁹ and A²² are each independently selected from N, CH and CY, any two A²⁰ and/or A²¹ are each independently selected from O, NH, NR^{AGG}, C═O, CH₂, CH(Y), CH(R^{AG}), C(Y)₂, C(Y)(R^{AG}) and C(R^{AG})₂, and each remaining A²⁰ and A²¹ is independently selected from O, NH, CH₂, CH(Y), and C(Y)₂.

In a further aspect of the eighth exemplary embodiment, A¹⁹ and A²² are each independently selected from N, CH and CF, one A²⁰ or A²¹ is independently selected from O, NH, NR^{AGG}, C═O, CH₂, CHF, CH(R^{AG}), CF₂, CF(R^{AG}) and C(R^{AG})₂, and each remaining A²⁰ and A²¹ is independently selected from O, NH, CH₂, CHF, and CF₂.

In one aspect of the eighth exemplary embodiment, A¹⁹ and A²² are each independently selected from N, CH, CY and CR^{AG}, and each A²⁰ and A²¹ is independently selected from NH, NR^{AGG}, C═O, CH₂, CH(Y), CH(R^{AG}), C(Y)₂, C(Y)(R^{AG}) and C(R^{AG})₂, such that ring Ag contains a single nitrogen atom in its ring structure. Typically in such an aspect, A¹⁹ and A²² are each independently selected from N, CH and CY, any two A²⁰ and/or A²¹ are each independently selected from NH, NR^{AGG}, C═O, CH₂, CH(Y), CH(R^{AG}), C(Y)₂, C(Y)(R^{AG}) and C(R^{AG})₂, and each remaining A²⁰ and A²¹ is independently selected from NH, CH₂, CH(Y), and C(Y)₂. In one embodiment of such an aspect, A¹⁹ is N. More typically, A¹⁹ is N, A²² is independently selected from CH and CF, one A²⁰ or A²¹ is independently selected from C═O, CH₂, CHF, CH(R^{AG}), CF₂, CF(R^{AG}) and C(R^{AG})₂, and each remaining A²⁰ and A²¹ is independently selected from CH₂, CHF, and CF₂.

In a further aspect of the eighth exemplary embodiment, A¹⁹ and A²² are each independently selected from N, CH and CY, and each A²⁰ and A²¹ is independently selected from NH, CH₂, CH(Y), and C(Y)₂, such that ring A^{g} contains a single nitrogen atom in its ring structure. In one embodiment of such an aspect, A¹⁹ is N. More typically, A¹⁹ is N, A²² is independently selected from CH and CF, and each A²⁰ and A²¹ is independently selected from CH₂, CHF, and CF₂.

In one aspect of the eighth exemplary embodiment, each R^{AG} is independently selected from a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{AG} contains, in total, from 1 to 10 carbon, nitrogen and oxygen atoms. Typically in such an aspect, each R^{AG} contains, in total, from 1 to 6 carbon, nitrogen and oxygen atoms.

In another aspect of the eighth exemplary embodiment, each R^{AG} is independently selected from a saturated hydrocarbyl group, wherein the saturated hydrocarbyl group is straight-chained or branched, or is or includes a cyclic group, wherein the saturated hydrocarbyl group optionally includes a single heteroatom O or N in its carbon skeleton, wherein the saturated hydrocarbyl group is optionally substituted with one or more fluoro groups and/or a single oxo (═O) group, and wherein each R^{AG} contains, in total, from 1 to 5 carbon, nitrogen and oxygen atoms. In one embodiment of such an aspect, each R^{AG} is independently selected from a saturated hydrocarbyl group, wherein the saturated hydrocarbyl group is straight-chained or branched, or is or includes a cyclic group, wherein the saturated hydrocarbyl group optionally includes a single heteroatom O or N in its carbon skeleton, wherein the saturated hydrocarbyl group is optionally fluoro-substituted, and wherein each R^{AG} contains, in total, from 1 to 4 carbon, nitrogen and oxygen atoms.

In one aspect of the eighth exemplary embodiment, each R^{AGG} contains, in total, from 1 to 6 carbon, nitrogen and oxygen atoms.

In another aspect of the eighth exemplary embodiment, each R^{AGG} is independently selected from a saturated hydrocarbyl group, wherein the saturated hydrocarbyl group is straight-chained or branched, or is or includes a cyclic group, wherein the saturated hydrocarbyl group optionally includes a single heteroatom O or N in its carbon skeleton, wherein the saturated hydrocarbyl group is optionally substituted with one or more fluoro groups and/or a single oxo (═O) group, and wherein each R^{AGG} contains, in total, from 1 to 5 carbon, nitrogen and oxygen atoms. Typically in such an aspect, each R^{AGG} is independently selected from a C₁-C₄ alkyl, C₁-C₄ fluoroalkyl, C₃-C₄ cycloalkyl or C₃-C₄ fluorocycloalkyl group.

In one aspect of any of the fourth to eighth exemplary embodiments, ring B is a 6-membered aryl ring or a 6-membered heteroaryl ring containing one or two nitrogen atoms in its ring structure. In one embodiment of such an aspect, ring B is a 6-membered heteroaryl ring containing one or two nitrogen atoms in its ring structure. For example, B¹ and B² may each be independently selected from C—H and C-Hal, B³ may be selected from N, C—H and C-Hal, and B⁴ may be N.

In a further aspect of any of the fourth to eighth exemplary embodiments, ring B is a 6-membered aryl ring or a 6-membered heteroaryl ring containing a single nitrogen atom in its ring structure. In one example of such an aspect, B¹, B² and B³ are each independently selected from C—H and C-Hal, and B⁴ is selected from N, C—H and C-Hal.

In one aspect of any of the fourth to eighth exemplary embodiments, each R^{B} is independently selected from a —CN, —R^{B1}, —OH, —OR^{B1}, —NH₂, —NHR^{B1} or —N(R^{B1})₂ group, wherein each R^{B1} is independently selected from a C₁-C₄ alkyl or C₁-C₄ fluoroalkyl group.

In another aspect of any of the fourth to eighth exemplary embodiments, n is 0 or 1. Typically in such an aspect, R^{B} where present is selected from a —CN, —R^{B1}, —OH, —OR^{B1}, —NH₂, —NHR^{B1} or —N(R^{B1})₂ group, wherein each R^{B1} is independently selected from a C₁-C₄ alkyl or C₁-C₄ fluoroalkyl group. More typically in such an aspect, R^{B} where present is selected from a methyl or fluoromethyl group.

In another aspect of any of the fourth to eighth exemplary embodiments, n is 0.

In one aspect of any of the fourth to eighth exemplary embodiments, each Hal is F.

In one aspect of any of the fourth to eighth exemplary embodiments, the atom of L² that is directly attached to ring B is O or N.

In one aspect of any of the fourth to eighth exemplary embodiments, L² is a straight-chained alkylene group, wherein the straight-chained alkylene group optionally includes one or two heteroatoms independently selected from O and N in its carbon skeleton, wherein L² has a chain length of from 2 to 8 atoms, and wherein L² may optionally be substituted with one or two oxo (═O) groups and/or with one or more (e.g. one, two, three or four) groups R^{L2}, wherein each R^{L2} is independently selected from a fluoro, C₁-C₄ alkyl, —O—(C₁-C₄ alkyl), C₁-C₄ fluoroalkyl or —O—(C₁-C₄ fluoroalkyl) group, or wherein any two R^{L2} may together with the atom(s) of the alkylene group to which they are attached form a monocyclic C₃-C₆ cycloalkyl or a monocyclic 4- to 6-membered saturated heterocyclic group, wherein the monocyclic C₃-C₆ cycloalkyl or the monocyclic 4- to 6-membered saturated heterocyclic group may optionally be substituted with one or more fluoro groups and/or one or two oxo (═O) groups. Typically in such an aspect, the straight-chained alkylene group includes one or two heteroatoms independently selected from O and N in its carbon skeleton. In one embodiment of such an aspect, the atom of L² that is directly attached to ring B is O. In another embodiment of such an aspect, the atom of L² that is directly attached to ring B is N.

In another aspect of any of the fourth to eighth exemplary embodiments, L² is a straight-chained alkylene or alkenylene group, wherein the straight-chained alkylene or alkenylene group optionally includes one or two heteroatoms independently selected from O and N in its carbon skeleton, wherein L² has a chain length of from 2 to 8 atoms, and wherein L² may optionally be substituted with one or two oxo (═O) groups and/or with one or more (e.g. one, two, three or four) groups R^{L2}, wherein each R^{L2} is independently selected from a fluoro, C₁-C₄ alkyl, —O—(C₁-C₄ alkyl), C₁-C₄ fluoroalkyl or —O—(C₁-C₄ fluoroalkyl) group, or wherein any two R^{L2} may together with the atoms of the alkylene or alkenylene group to which they are attached form a phenyl or a 5- or 6-membered heteroaryl group (such as a pyridinyl group), wherein the phenyl or the 5- or 6-membered heteroaryl group may optionally be substituted with one or more fluoro groups. Typically in such an aspect, the straight-chained alkylene or alkenylene group includes one or two heteroatoms independently selected from O and N in its carbon skeleton. In one embodiment of such an aspect, the atom of L² that is directly attached to ring B is O. In another embodiment of such an aspect, the atom of L² that is directly attached to ring B is N.

In a further aspect of any of the fourth to eighth exemplary embodiments, L² is a straight-chained alkylene group, wherein the straight-chained alkylene group includes one or two heteroatoms independently selected from O and N in its carbon skeleton, wherein L² has a chain length of from 2 to 8 atoms, and wherein L² may optionally be substituted with one oxo (═O) group and/or with one, two, three or four groups R^{L2}, wherein each R^{L2} is independently selected from a fluoro, methyl or fluoromethyl group, or wherein any two R^{L2} attached to the same carbon atom may together with the carbon atom to which they are attached form a cyclopropyl group, wherein the cyclopropyl group may optionally be fluoro-substituted. In one embodiment of such an aspect, the atom of L² that is directly attached to ring B is O. In another embodiment of such an aspect, the atom of L² that is directly attached to ring B is N.

Typically in accordance with any aspect of any of the fourth to eighth exemplary embodiments, L² has a chain length of from 3 to 6 atoms.

Typically in accordance with any aspect of any of the fourth to eighth exemplary embodiments, L² contains in total from 2 to 15 carbon, nitrogen and oxygen atoms. More typically, L² contains in total from 3 to 10 carbon, nitrogen and oxygen atoms.

In one aspect of any of the fourth to eighth exemplary embodiments, R⁴ is selected from a C₁-C₄ alkyl, C₁-C₄ fluoroalkyl, C₃-C₆ cycloalkyl or C₃-C₆ fluorocycloalkyl group, and R⁵ is selected from hydrogen, F or a methyl or fluoromethyl group. Typically in such an aspect, R⁵ is hydrogen or F.

In another aspect of any of the fourth to eighth exemplary embodiments, R⁴ is selected from a C₃-C₄ alkyl, C₃-C₄ fluoroalkyl, C₃-C₅ cycloalkyl or C₃-C₅ fluorocycloalkyl group, and R⁵ is selected from hydrogen, F or a methyl or fluoromethyl group. Typically in such an aspect, R⁵ is hydrogen or F.

In yet another aspect of any of the fourth to eighth exemplary embodiments, R⁴ and R⁵ together form a divalent group selected from —CH₂CH₂CH₂—, —CH₂CH₂O— and —OCH₂CH₂—, wherein the divalent group formed by R⁴ and R⁵ may optionally be fluoro-substituted.

In one aspect of any of the fourth to eighth exemplary embodiments, R⁶ is selected from hydrogen, F, or a —CN, methyl, fluoromethyl, —OC(R²⁰)₃ or —C(R²⁰)₂—OC(R²⁰)₃ group (wherein R²⁰ is as previously defined), and R⁷ is selected from hydrogen, F, or a methyl or fluoromethyl group. Typically in such an aspect, R⁶ is selected from hydrogen, F, or a —CN, methyl, fluoromethyl, —OMe or —O-(fluoromethyl) group, and R⁷ is selected from hydrogen, F, or a methyl or fluoromethyl group.

In another aspect of any of the fourth to eighth exemplary embodiments, R⁶ and R⁷ are each independently selected from hydrogen, F, or a methyl or fluoromethyl group. Typically in such an aspect, R⁶ is hydrogen or F and R⁷ is hydrogen, F, or a methyl or fluoromethyl group.

Typically in accordance with any aspect of any of the fourth to eighth exemplary embodiments, at least one of R⁵, R⁶ or R⁷ is selected from hydrogen or F. More typically, at least one of R⁶ or R⁷ is selected from hydrogen or F.

In one embodiment of the first aspect of the invention, any compound of formula (I), (Ia), (Ib), (Ic), (Id), (Ie), (If) or (Ig) contains from 10 to 80 atoms other than hydrogen or halogen. More typically, any compound of formula (I), (Ia), (Ib), (Ic), (Id), (Ie), (If) or (Ig) contains from 15 to 60 atoms other than hydrogen or halogen. Yet more typically, any compound of formula (I), (Ia), (Ib), (Ic), (Id), (Ie), (If) or (Ig) contains from 20 to 50 atoms other than hydrogen or halogen. Yet more typically, any compound of formula (I), (Ia), (Ib), (Ic), (Id), (Ie), (If) or (Ig) contains from 22 to 45 atoms other than hydrogen or halogen. More typically still, any compound of formula (I), (Ia), (Ib), (Ic), (Id), (Ie), (If) or (Ig) contains from 25 to 40 atoms other than hydrogen or halogen.

In one aspect of any of the above embodiments, the compound of formula (I), (Ia), (Ib), (Ic), (Id), (Ie), (If) or (Ig) has a molecular weight of from 250 to 2000 Da. Typically, the compound of formula (I), (Ia), (Ib), (Ic), (Id), (Ie), (If) or (Ig) has a molecular weight of from 275 to 900 Da. More typically, the compound of formula (I), (Ia), (Ib), (Ic), (Id), (Ie), (If) or (Ig) has a molecular weight of from 280 to 700 Da. More typically still, the compound of formula (I), (Ia), (Ib), (Ic), (Id), (Ie), (If) or (Ig) has a molecular weight of from 300 to 600 Da.

A second aspect of the invention provides a compound selected from the group consisting of:

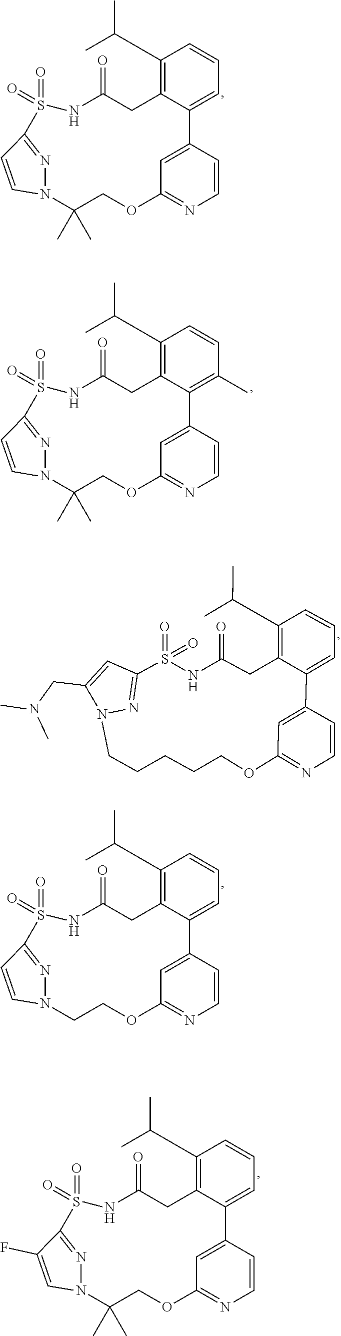

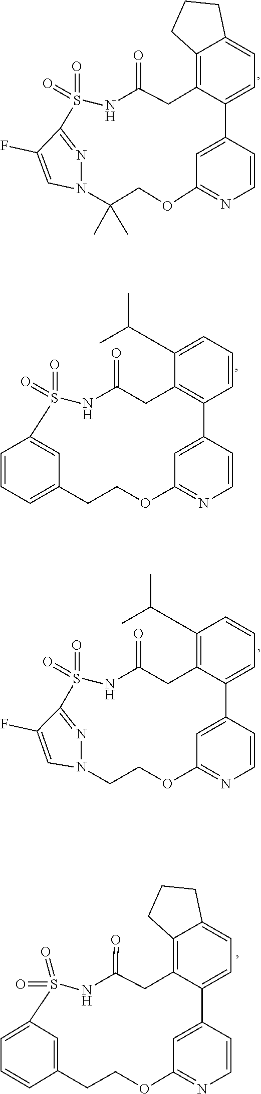 [SMILES: CC1(C)COc2cc(ccn2)-c2ccc3c(c2CC(=O)NS(=O)(=O)c2nn1cc2F)CCC3 ; CC(C)c1cccc2c1CC(=O)NS(=O)(=O)c1cccc(c1)CCOc1cc-2ccn1 ; CC(C)c1cccc2c1CC(=O)NS(=O)(=O)c1nn(cc1F)CCOc1cc-2ccn1 ; O=C1Cc2c(ccc3c2CCC3)-c2ccnc(c2)OCCc2cccc(c2)S(=O)(=O)N1]

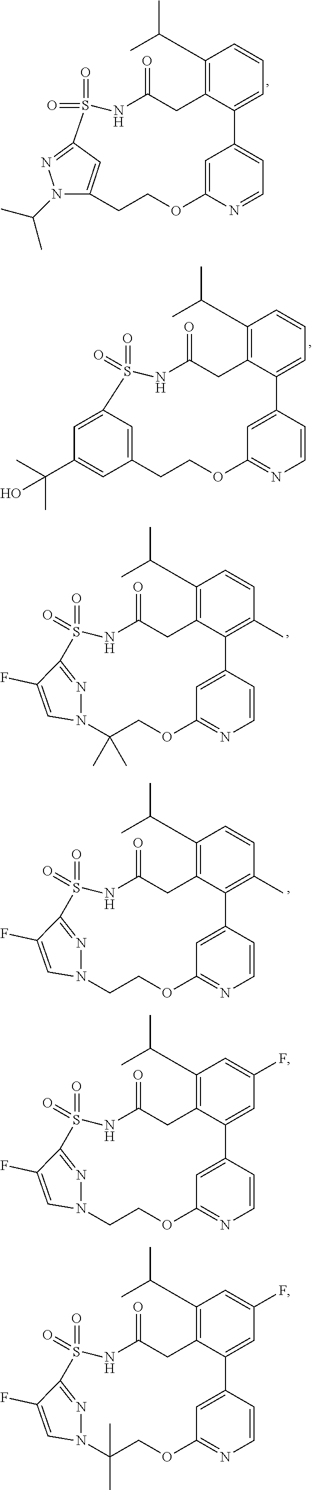

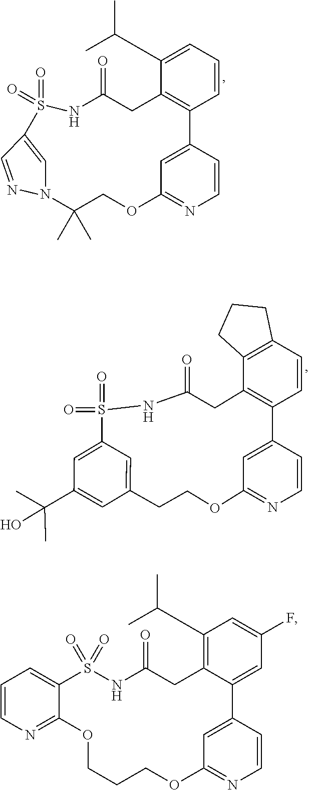 [SMILES: CC(C)c1cccc2c1CC(=O)NS(=O)(=O)c1cnn(c1)C(C)(C)COc1cc-2ccn1 ; CC(C)(O)c1cc2cc(c1)S(=O)(=O)NC(=O)Cc1c(ccc3c1CCC3)-c1ccnc(c1)OCC2 ; CC(C)c1cc(F)cc2c1CC(=O)NS(=O)(=O)c1cccnc1OCCCOc1cc-2ccn1]

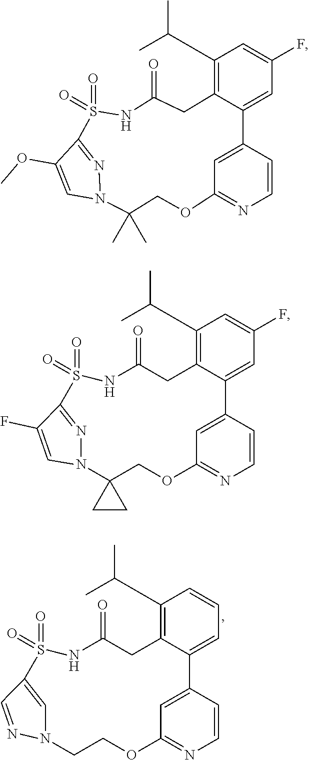

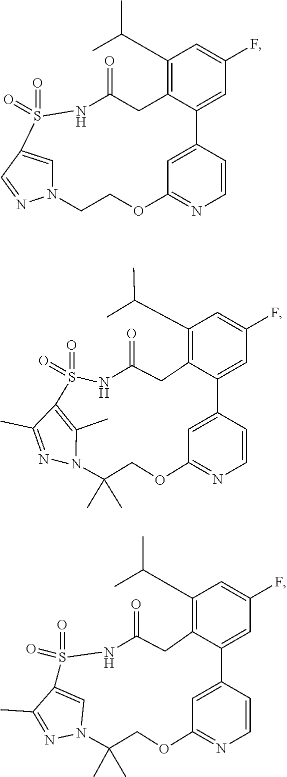

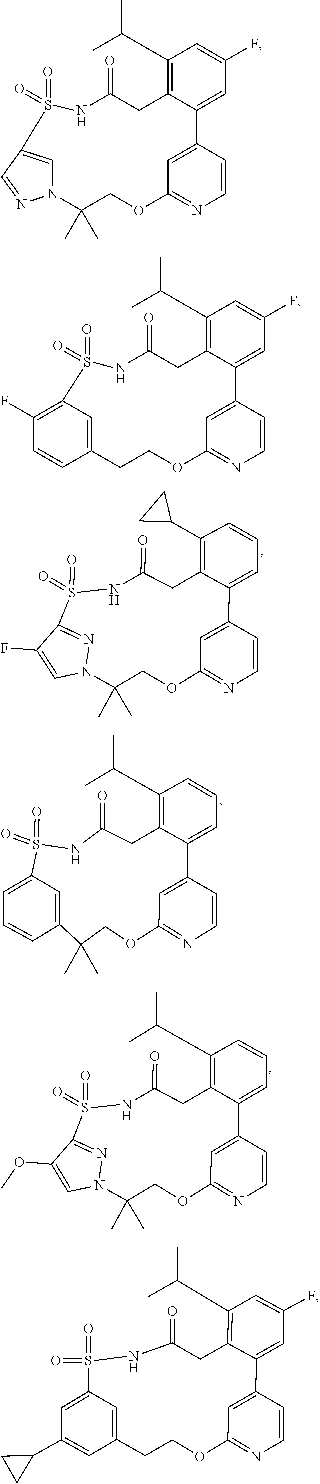

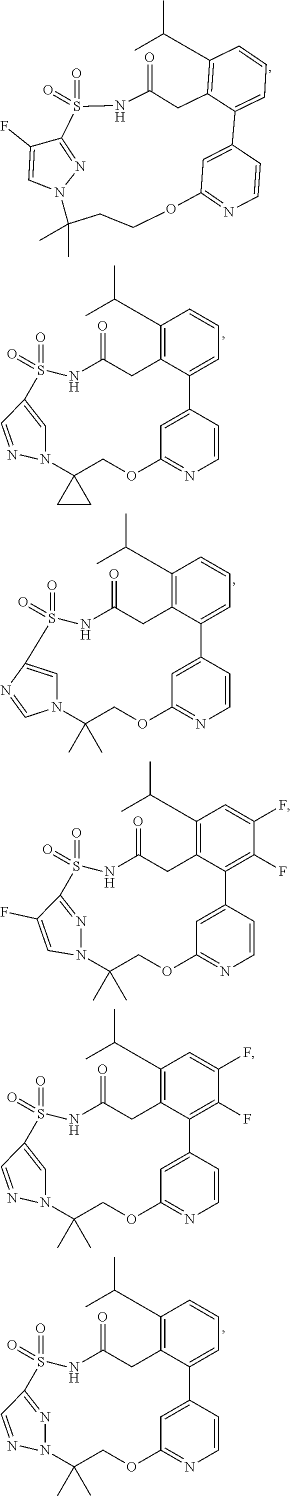

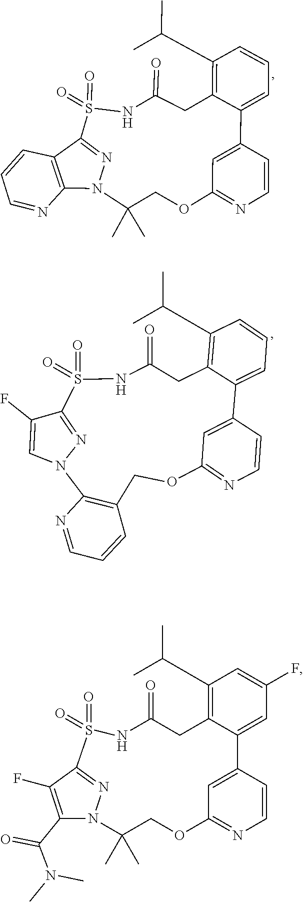

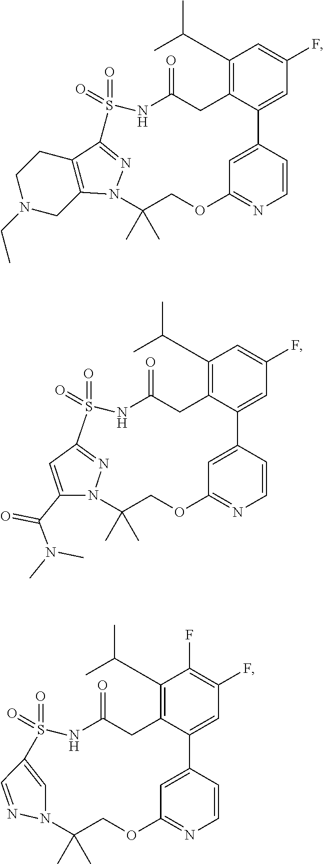 [SMILES: CCN1CCc2c3nn(c2C1)C(C)(C)COc1cc(ccn1)-c1cc(F)cc(C(C)C)c1CC(=O)NS3(=O)=O ; CC(C)c1cc(F)cc2c1CC(=O)NS(=O)(=O)c1cc(C(=O)N(C)C)n(n1)C(C)(C)COc1cc-2ccn1 ; CC(C)c1c(F)c(F)cc2c1CC(=O)NS(=O)(=O)c1cnn(c1)C(C)(C)COc1cc-2ccn1]

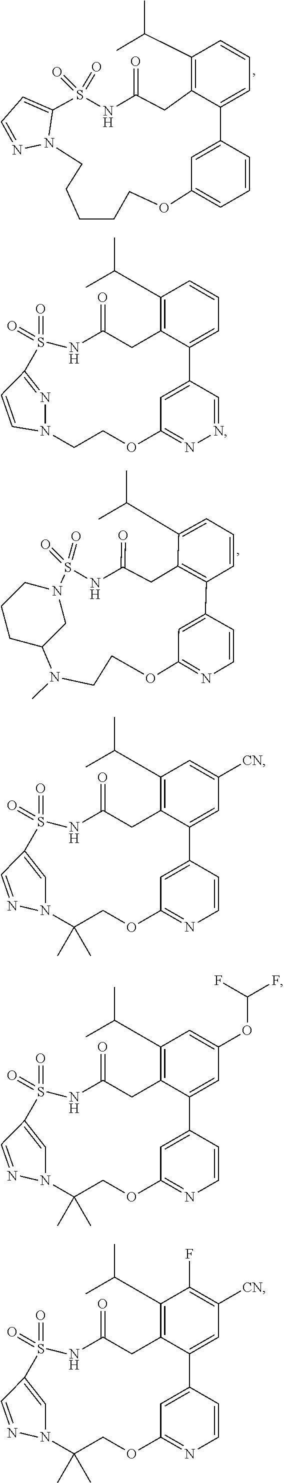

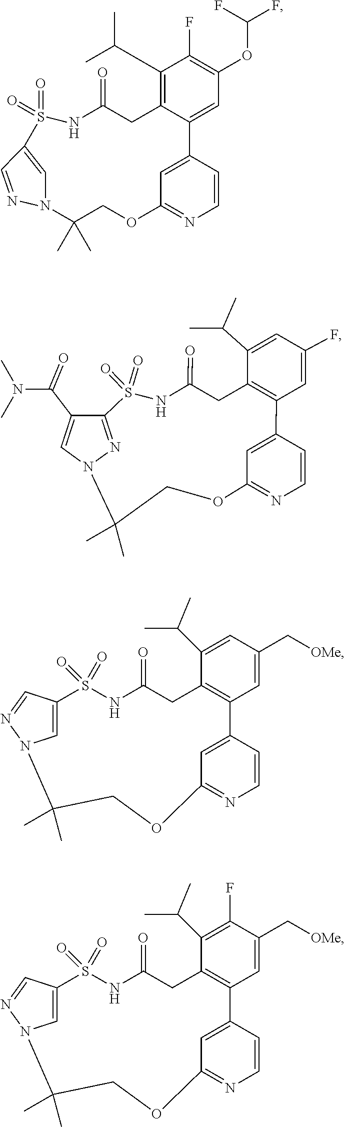 [SMILES: CC(C)c1c(F)c(OC(F)F)cc2c1CC(=O)NS(=O)(=O)c1cnn(c1)C(C)(C)COc1cc-2ccn1 ; CC(C)c1cc(F)cc2c1CC(=O)NS(=O)(=O)c1nn(cc1C(=O)N(C)C)C(C)(C)COc1cc-2ccn1 ; COCc1cc2c(c(C(C)C)c1)CC(=O)NS(=O)(=O)c1cnn(c1)C(C)(C)COc1cc-2ccn1 ; COCc1cc2c(c(C(C)C)c1F)CC(=O)NS(=O)(=O)c1cnn(c1)C(C)(C)COc1cc-2ccn1]

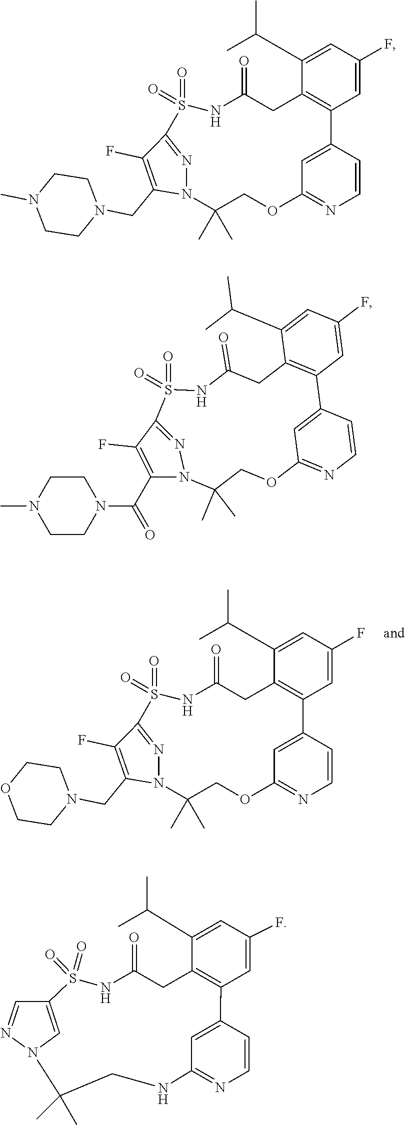

A third aspect of the invention provides a pharmaceutically acceptable salt, solvate or prodrug of any compound of the first or second aspect of the invention.

The compounds of the present invention can be used both, in their free base form and their acid addition salt form. For the purposes of this invention, a “salt” of a compound of the present invention includes an acid addition salt. Acid addition salts are preferably pharmaceutically acceptable, non-toxic addition salts with suitable acids, including but not limited to inorganic acids such as hydrohalogenic acids (for example, hydrofluoric, hydrochloric, hydrobromic or hydroiodic acid) or other inorganic acids (for example, nitric, perchloric, sulfuric or phosphoric acid); or organic acids such as organic carboxylic acids (for example, propionic, butyric, glycolic, lactic, mandelic, citric, acetic, benzoic, salicylic, succinic, malic or hydroxysuccinic, tartaric, fumaric, maleic, hydroxymaleic, mucic or galactaric, gluconic, pantothenic or pamoic acid), organic sulfonic acids (for example, methanesulfonic, trifluoromethanesulfonic, ethanesulfonic, 2-hydroxyethanesulfonic, benzenesulfonic, toluene-p-sulfonic, naphthalene-2-sulfonic or camphorsulfonic acid) or amino acids (for example, ornithinic, glutamic or aspartic acid). The acid addition salt may be a mono-, di-, tri- or multi-acid addition salt. A preferred salt is a hydrohalogenic, sulfuric, phosphoric or organic acid addition salt. A preferred salt is a hydrochloric acid addition salt.

Where a compound of the invention includes a quaternary ammonium group, typically the compound is used in its salt form. The counter ion to the quaternary ammonium group may be any pharmaceutically acceptable, non-toxic counter ion. Examples of suitable counter ions include the conjugate bases of the protic acids discussed above in relation to acid addition salts.

The compounds of the present invention can also be used both, in their free acid form and their salt form. For the purposes of this invention, a “salt” of a compound of the present invention includes one formed between a protic acid functionality (such as a carboxylic acid group) of a compound of the present invention and a suitable cation. Suitable cations include, but are not limited to lithium, sodium, potassium, magnesium, calcium and ammonium. The salt may be a mono-, di-, tri- or multi-salt. Preferably the salt is a mono- or di-lithium, sodium, potassium, magnesium, calcium or ammonium salt. More preferably the salt is a mono- or di-sodium salt or a mono- or di-potassium salt.

Preferably any salt is a pharmaceutically acceptable non-toxic salt. However, in addition to pharmaceutically acceptable salts, other salts are included in the present invention, since they have potential to serve as intermediates in the purification or preparation of other, for example, pharmaceutically acceptable salts, or are useful for identification, characterisation or purification of the free acid or base.

The compounds and/or salts of the present invention may be anhydrous or in the form of a hydrate (e.g. a hemihydrate, monohydrate, dihydrate or trihydrate) or other solvate. Such other solvates may be formed with common organic solvents, including but not limited to, alcoholic solvents e.g. methanol, ethanol or isopropanol.

In some embodiments of the present invention, therapeutically inactive prodrugs are provided. Prodrugs are compounds which, when administered to a subject such as a human, are converted in whole or in part to a compound of the invention. In most embodiments, the prodrugs are pharmacologically inert chemical derivatives that can be converted in vivo to the active drug molecules to exert a therapeutic effect. Any of the compounds described herein can be administered as a prodrug to increase the activity, bioavailability, or stability of the compound or to otherwise alter the properties of the compound. Typical examples of prodrugs include compounds that have biologically labile protecting groups on a functional moiety of the active compound.

Prodrugs include, but are not limited to, compounds that can be oxidized, reduced, aminated, deaminated, hydroxylated, dehydroxylated, hydrolyzed, dehydrolyzed, alkylated, dealkylated, acylated, deacylated, phosphorylated, and/or dephosphorylated to produce the active compound. The present invention also encompasses salts and solvates of such prodrugs as described above.

The compounds, salts, solvates and prodrugs of the present invention may contain at least one chiral centre. The compounds, salts, solvates and prodrugs may therefore exist in at least two isomeric forms. The present invention encompasses racemic mixtures of the compounds, salts, solvates and prodrugs of the present invention as well as enantiomerically enriched and substantially enantiomerically pure isomers. For the purposes of this invention, a “substantially enantiomerically pure” isomer of a compound comprises less than 5% of other isomers of the same compound, more typically less than 2%, and most typically less than 0.5% by weight.

The compounds, salts, solvates and prodrugs of the present invention may contain any stable isotope including, but not limited to ¹²C, ¹³C, ¹H, ²H (D), ¹⁴N, ¹⁵N, ¹⁶O, ¹⁷O, ¹⁸O, ¹⁹F and ¹²⁷I, and any radioisotope including, but not limited to ¹¹C, ¹⁴C, ³H (T), ¹³N, ¹⁵O, ¹⁸F, ¹²³I, ¹²⁴I, ¹²⁵I and ¹³¹I.

The compounds, salts, solvates and prodrugs of the present invention may be in any polymorphic or amorphous form.

A fourth aspect of the invention provides a pharmaceutical composition comprising a compound of the first or second aspect of the invention, or a pharmaceutically acceptable salt, solvate or prodrug of the third aspect of the invention, and a pharmaceutically acceptable excipient.

Conventional procedures for the selection and preparation of suitable pharmaceutical formulations are described in, for example, “Aulton's Pharmaceutics—The Design and Manufacture of Medicines”, M. E. Aulton and K. M. G. Taylor, Churchill Livingstone Elsevier, 4^{th} Ed., 2013.

Pharmaceutically acceptable excipients including adjuvants, diluents or carriers that may be used in the pharmaceutical compositions of the invention are those conventionally employed in the field of pharmaceutical formulation, and include, but are not limited to, sugars, sugar alcohols, starches, ion exchangers, alumina, aluminium stearate, lecithin, serum proteins such as human serum albumin, buffer substances such as phosphates, glycerine, sorbic acid, potassium sorbate, partial glyceride mixtures of saturated vegetable fatty acids, water, salts or electrolytes such as protamine sulfate, disodium hydrogen phosphate, potassium hydrogen phosphate, sodium chloride, zinc salts, colloidal silica, magnesium trisilicate, polyvinylpyrrolidone, cellulose-based substances, polyethylene glycol, sodium carboxymethylcellulose, polyacrylates, waxes, polyethylene-polyoxypropylene-block polymers, polyethylene glycol and wool fat.

In one embodiment, the pharmaceutical composition of the fourth aspect of the invention additionally comprises one or more further active agents.

In a further embodiment, the pharmaceutical composition of the fourth aspect of the invention may be provided as a part of a kit of parts, wherein the kit of parts comprises the pharmaceutical composition of the fourth aspect of the invention and one or more further pharmaceutical compositions, wherein the one or more further pharmaceutical compositions each comprise a pharmaceutically acceptable excipient and one or more further active agents.

A fifth aspect of the invention provides a compound of the first or second aspect of the invention, or a pharmaceutically acceptable salt, solvate or prodrug of the third aspect of the invention, or a pharmaceutical composition of the fourth aspect of the invention, for use in medicine, and/or for use in the treatment or prevention of a disease, disorder or condition. Typically, the use comprises the administration of the compound, salt, solvate, prodrug or pharmaceutical composition to a subject. In one embodiment, the use comprises the co-administration of one or more further active agents.

The term “treatment” as used herein refers equally to curative therapy, and ameliorating or palliative therapy. The term includes obtaining beneficial or desired physiological results, which may or may not be established clinically. Beneficial or desired clinical results include, but are not limited to, the alleviation of symptoms, the prevention of symptoms, the diminishment of extent of disease, the stabilisation (i.e., not worsening) of a condition, the delay or slowing of progression/worsening of a condition/symptom, the amelioration or palliation of a condition/symptom, and remission (whether partial or total), whether detectable or undetectable. The term “palliation”, and variations thereof, as used herein, means that the extent and/or undesirable manifestations of a physiological condition or symptom are lessened and/or time course of the progression is slowed or lengthened, as compared to not administering a compound, salt, solvate, prodrug or pharmaceutical composition of the present invention. The term “prevention” as used herein in relation to a disease, disorder or condition, relates to prophylactic or preventative therapy, as well as therapy to reduce the risk of developing the disease, disorder or condition. The term “prevention” includes both the avoidance of occurrence of the disease, disorder or condition, and the delay in onset of the disease, disorder or condition. Any statistically significant (p≤0.05) avoidance of occurrence, delay in onset or reduction in risk as measured by a controlled clinical trial may be deemed a prevention of the disease, disorder or condition. Subjects amenable to prevention include those at heightened risk of a disease, disorder or condition as identified by genetic or biochemical markers. Typically, the genetic or biochemical markers are appropriate to the disease, disorder or condition under consideration and may include for example, inflammatory biomarkers such as C-reactive protein (CRP) and monocyte chemoattractant protein 1 (MCP-1) in the case of inflammation; total cholesterol, triglycerides, insulin resistance and C-peptide in the case of NAFLD and NASH; and more generally IL-1β and IL-18 in the case of a disease, disorder or condition responsive to NLRP3 inhibition.

A sixth aspect of the invention provides the use of a compound of the first or second aspect, or a pharmaceutically effective salt, solvate or prodrug of the third aspect, in the manufacture of a medicament for the treatment or prevention of a disease, disorder or condition. Typically, the treatment or prevention comprises the administration of the compound, salt, solvate, prodrug or medicament to a subject. In one embodiment, the treatment or prevention comprises the co-administration of one or more further active agents.

A seventh aspect of the invention provides a method of treatment or prevention of a disease, disorder or condition, the method comprising the step of administering an effective amount of a compound of the first or second aspect, or a pharmaceutically acceptable salt, solvate or prodrug of the third aspect, or a pharmaceutical composition of the fourth aspect, to thereby treat or prevent the disease, disorder or condition. In one embodiment, the method further comprises the step of co-administering an effective amount of one or more further active agents. Typically, the administration is to a subject in need thereof.

An eighth aspect of the invention provides a compound of the first or second aspect of the invention, or a pharmaceutically acceptable salt, solvate or prodrug of the third aspect of the invention, or a pharmaceutical composition of the fourth aspect of the invention, for use in the treatment or prevention of a disease, disorder or condition in an individual, wherein the individual has a germline or somatic non-silent mutation in NLRP3. The mutation may be, for example, a gain-of-function or other mutation resulting in increased NLRP3 activity. Typically, the use comprises the administration of the compound, salt, solvate, prodrug or pharmaceutical composition to the individual. In one embodiment, the use comprises the co-administration of one or more further active agents. The use may also comprise the diagnosis of an individual having a germline or somatic non-silent mutation in NLRP3, wherein the compound, salt, solvate, prodrug or pharmaceutical composition is administered to an individual on the basis of a positive diagnosis for the mutation. Typically, identification of the mutation in NLRP3 in the individual may be by any suitable genetic or biochemical means.

A ninth aspect of the invention provides the use of a compound of the first or second aspect, or a pharmaceutically effective salt, solvate or prodrug of the third aspect, in the manufacture of a medicament for the treatment or prevention of a disease, disorder or condition in an individual, wherein the individual has a germline or somatic non-silent mutation in NLRP3. The mutation may be, for example, a gain-of-function or other mutation resulting in increased NLRP3 activity. Typically, the treatment or prevention comprises the administration of the compound, salt, solvate, prodrug or medicament to the individual. In one embodiment, the treatment or prevention comprises the co-administration of one or more further active agents. The treatment or prevention may also comprise the diagnosis of an individual having a germline or somatic non-silent mutation in NLRP3, wherein the compound, salt, solvate, prodrug or medicament is administered to an individual on the basis of a positive diagnosis for the mutation. Typically, identification of the mutation in NLRP3 in the individual may be by any suitable genetic or biochemical means.

A tenth aspect of the invention provides a method of treatment or prevention of a disease, disorder or condition, the method comprising the steps of diagnosing an individual as having a germline or somatic non-silent mutation in NLRP3, and administering an effective amount of a compound of the first or second aspect, or a pharmaceutically acceptable salt, solvate or prodrug of the third aspect, or a pharmaceutical composition of the fourth aspect, to the positively diagnosed individual, to thereby treat or prevent the disease, disorder or condition. In one embodiment, the method further comprises the step of co-administering an effective amount of one or more further active agents. Typically, the administration is to a subject in need thereof.

In general embodiments, the disease, disorder or condition may be a disease, disorder or condition of the immune system, the cardiovascular system, the endocrine system, the gastrointestinal tract, the renal system, the hepatic system, the metabolic system, the respiratory system, the central nervous system, may be a cancer or other malignancy, and/or may be caused by or associated with a pathogen.

It will be appreciated that these general embodiments defined according to broad categories of diseases, disorders and conditions are not mutually exclusive. In this regard any particular disease, disorder or condition may be categorized according to more than one of the above general embodiments. A non-limiting example is type I diabetes which is an autoimmune disease and a disease of the endocrine system.

In one embodiment of the fifth, sixth, seventh, eighth, ninth or tenth aspect of the invention, the disease, disorder or condition is responsive to NLRP3 inhibition. As used herein, the term “NLRP3 inhibition” refers to the complete or partial reduction in the level of activity of NLRP3 and includes, for example, the inhibition of active NLRP3 and/or the inhibition of activation of NLRP3.

There is evidence for a role of NLRP3-induced IL-1 and IL-18 in the inflammatory responses occurring in connection with, or as a result of, a multitude of different disorders (Menu et al., Clinical and Experimental Immunology, 166: 1-15, 2011; Strowig et al., Nature, 481: 278-286, 2012).

Genetic diseases in which a role for NLRP3 has been suggested include sickle cell disease (Vogel et al., Blood, 130 (Suppl 1): 2234, 2017), and Valosin Containing Protein disease (Nalbandian et al., Inflammation, 40(1): 21-41, 2017).

NLRP3 has been implicated in a number of autoinflammatory diseases, including Familial Mediterranean fever (FMF), TNF receptor associated periodic syndrome (TRAPS), hyperimmunoglobulinemia D and periodic fever syndrome (HIDS), pyogenic arthritis, pyoderma gangrenosum and acne (PAPA), Sweet's syndrome, chronic nonbacterial osteomyelitis (CNO), and acne vulgaris (Cook et al., Eur J Immunol, 40: 595-653, 2010). In particular, NLRP3 mutations have been found to be responsible for a set of rare autoinflammatory diseases known as CAPS (Ozaki et al., J Inflammation Research, 8: 15-27, 2015; Schroder et al., Cell, 140: 821-832, 2010; and Menu et al., Clinical and Experimental Immunology, 166: 1-15, 2011). CAPS are heritable diseases characterized by recurrent fever and inflammation and are comprised of three autoinflammatory disorders that form a clinical continuum. These diseases, in order of increasing severity, are familial cold autoinflammatory syndrome (FCAS), Muckle-Wells syndrome (MWS), and chronic infantile cutaneous neurological articular syndrome (CINCA; also called neonatal-onset multisystem inflammatory disease, NOMID), and all have been shown to result from gain-of-function mutations in the NLRP3 gene, which leads to increased secretion of IL-1β.

A number of autoimmune diseases have been shown to involve NLRP3 including, in particular, multiple sclerosis, type 1 diabetes (T1D), psoriasis, rheumatoid arthritis (RA), Behcet's disease, Schnitzler's syndrome, macrophage activation syndrome, Coeliac disease (Masters, Clin Immunol, 147(3): 223-228, 2013; Braddock et al., Nat Rev Drug Disc, 3: 1-10, 2004; Inoue et al., Immunology, 139: 11-18, 2013; Coll et al., Nat Med, 21(3): 248-55, 2015; Scott et al., Clin Exp Rheumatol, 34(1): 88-93, 2016; Pontillo et al., Autoimmunity, 43(8): 583-589, 2010; and Guo et al., Clin Exp Immunol, 194(2): 231-243, 2018), systemic lupus erythematosus (Lu et al., J Immunol, 198(3): 1119-29, 2017) including lupus nephritis (Zhao et al., Arthritis and Rheumatism, 65(12): 3176-3185, 2013), multiple sclerosis (Xu et al., J Cell Biochem, 120(4): 5160-5168, 2019), and systemic sclerosis (Artlett et al., Arthritis Rheum, 63(11): 3563-74, 2011).

NLRP3 has also been shown to play a role in a number of respiratory and lung diseases including chronic obstructive pulmonary disorder (COPD), asthma (including steroid-resistant asthma and eosinophilic asthma), bronchitis, asbestosis, volcanic ash induced inflammation, and silicosis (Cassel et al., Proceedings of the National Academy of Sciences, 105(26): 9035-9040, 2008; Chen et al., ERJ Open Research, 4: 00130-2017, 2018; Chen et al., Toxicological Sciences, 170(2): 462-475, 2019; Damby et al., Front Immun, 8: 2000, 2018; De Nardo et al., Am J Pathol, 184: 42-54, 2014; Lv et al., J Biol Chem, 293(48): 18454, 2018; and Kim et al., Am J Respir Crit Care Med, 196(3): 283-97, 2017).

NLRP3 has also been suggested to have a role in a number of central nervous system conditions, including Parkinson's disease (PD), Alzheimer's disease (AD), dementia, Huntington's disease, cerebral malaria, brain injury from pneumococcal meningitis (Walsh et al., Nature Reviews, 15: 84-97, 2014; Cheng et al., Autophagy, 1-13, 2020; Couturier et al., J Neuroinflamm, 13: 20, 2016; and Dempsey et al., Brain Behav Immun, 61: 306-316, 2017), intracranial aneurysms (Zhang et al., J Stroke & Cerebrovascular Dis, 24(5): 972-979, 2015), intracerebral haemorrhages (ICH) (Ren et al., Stroke, 49(1): 184-192, 2018), cerebral ischemia-reperfusion injuries (Fauzia et al., Front Pharmacol, 9: 1034, 2018; Hong et al., Neural Plasticity, 2018: 8, 2018; Ye et al., Experimental Neurology, 292: 46-55, 2017), general anesthesia neuroinflammation (Fan et al., Front Cell Neurosci, 12: 426, 2018), sepsis-associated encephalopathy (SAE) (Fu et al., Inflammation, 42(1): 306-318, 2019), perioperative neurocognitive disorders including postoperative cognitive dysfunction (POCD) (Fan et al., Front Cell Neurosci, 12: 426, 2018; and Fu et al., International Immunopharmacology, 82: 106317, 2020), early brain injury (subarachnoid haemorrhage SAH) (Luo et al., Brain Res Bull, 146: 320-326, 2019), and traumatic brain injury (Ismael et al., J Neurotrauma, 35(11): 1294-1303, 2018; and Chen et al., Brain Research, 1710: 163-172, 2019).

NRLP3 activity has also been shown to be involved in various metabolic diseases including type 2 diabetes (T2D), atherosclerosis, obesity, gout, pseudo-gout, metabolic syndrome (Wen et al., Nature Immunology, 13: 352-357, 2012; Duewell et al., Nature, 464: 1357-1361, 2010; Strowig et al., Nature, 481: 278-286, 2012), and non-alcoholic steatohepatitis (NASH) (Mridha et al., J Hepatol, 66(5): 1037-46, 2017).

A role for NLRP3 via IL-1β has also been suggested in atherosclerosis (Chen et al., Journal of the American Heart Association, 6(9): e006347, 2017; and Chen et al., Biochem Biophys Res Commun, 495(1): 382-387, 2018), myocardial infarction (van Hout et al., Eur Heart J, 38(11): 828-36, 2017), cardiovascular disease (Janoudi et al., European Heart Journal, 37(25): 1959-1967, 2016), cardiac hypertrophy and fibrosis (Gan et al., Biochim Biophys Acta, 1864(1): 1-10, 2018), heart failure (Sano et al., J Am Coll Cardiol, 71(8): 875-66, 2018), aortic aneurysm and dissection (Wu et al., Arterioscler Thromb Vase Biol, 37(4): 694-706, 2017), cardiac injury induced by metabolic dysfunction (Pavillard et al., Oncotarget, 8(59): 99740-99756, 2017; and Zhang et al., Biochimica et Biophysica Acta, 1863(6): 1556-1567, 2017), atrial fibrillation (Yao et al., Circulation, 138(20): 2227-2242, 2018), hypertension (Gan et al., Biochim Biophys Acta, 1864(1): 1-10, 2018), and other cardiovascular events (Ridker et al., N Engl J Med, doi: 10.1056/NEJMoa1707914, 2017).

Other diseases, disorders and conditions in which NLRP3 has been shown to be involved include:
ocular diseases such as both wet and dry age-related macular degeneration (Doyle et al., Nature Medicine, 18: 791-798, 2012; and Tarallo et al., Cell, 149(4): 847-59, 2012), diabetic retinopathy (Loukovaara et al., Acta Ophthalmol, 95(8): 803-808, 2017) and optic nerve damage (Puyang et al., Sci Rep, 6: 20998, 2016 Feb. 19);
liver diseases including non-alcoholic steatohepatitis (NASH) (Henao-Meija et al., Nature, 482: 179-185, 2012), ischemia reperfusion injury of the liver (Yu et al., Transplantation, 103(2): 353-362, 2019), fulminant hepatitis (Pourcet et al., Gastroenterology, 154(5): 1449-1464, e20, 2018), liver fibrosis (Zhang et al., Parasit Vectors, 12(1): 29, 2019), and liver failure including acute liver failure (Wang et al., Hepatol Res, 48(3): E194-E202, 2018);
kidney diseases including nephrocalcinosis (Anders et al., Kidney Int, 93(3): 656-669, 2018), kidney fibrosis including chronic crystal nephropathy (Ludwig-Portugall et al., Kidney Int, 90(3): 525-39, 2016), obesity related glomerulopathy (Zhao et al., Mediators of Inflammation, article 3172647, 2019), acute kidney injury (Zhang et al., Diabetes, Metabolic Syndrome and Obesity: Targets and Therapy, 12: 1297-1309, 2019), and renal hypertension (Krishnan et al., Br J Pharmacol, 173(4): 752-65, 2016; Krishnan et al., Cardiovasc Res, 115(4): 776-787, 2019; Dinh et al., Aging, 9(6): 1595-1606, 2017);
conditions associated with diabetes including diabetic encephalopathy (Zhai et al., Molecules, 23(3): 522, 2018), diabetic retinopathy (Zhang et al., Cell Death Dis, 8(7): e2941, 2017), diabetic nephropathy (also called diabetic kidney disease) (Chen et al., BMC Complementary and Alternative Medicine, 18: 192, 2018), and diabetic hypoadiponectinemia (Zhang et al., Biochimica et Biophysica Acta (BBA)—Molecular Basis of Disease, 1863(6): 1556-1567, 2017);
inflammatory reactions in the lung and skin (Primiano et al., J Immunol, 197(6): 2421-33, 2016) including lung ischemia-reperfusion injury (Xu et al., Biochemical and Biophysical Research Communications, 503(4): 3031-3037, 2018), epithelial to mesenchymal transition (EMT) (Li et al., Experimental Cell Research, 362(2): 489-497, 2018), contact hypersensitivity (such as bullous pemphigoid (Fang et al., J Dermatol Sci, 83(2): 116-23, 2016)), atopic dermatitis (Niebuhr et al., Allergy, 69(8): 1058-67, 2014), Hidradenitis suppurativa (Alikhan et al., J Am Acad Dermatol, 60(4): 539-61, 2009), acne vulgaris (Qin et al., J Invest Dermatol, 134(2): 381-88, 2014), and sarcoidosis (Jager et al., Am J Respir Crit Care Med, 191: A5816, 2015);
inflammatory reactions in the joints (Braddock et al., Nat Rev Drug Disc, 3: 1-10, 2004) and osteoarthritis (Jin et al., PNAS, 108(36): 14867-14872, 2011);
conditions associated with arthritis including arthritic fever (Verma, Linköping University Medical Dissertations, No. 1250, 2011);
amyotrophic lateral sclerosis (Gugliandolo et al., Inflammation, 41(1): 93-103, 2018);
cystic fibrosis (Iannitti et al., Nat Commun, 7: 10791, 2016);
stroke (Walsh et al., Nature Reviews, 15: 84-97, 2014; Ye et al., Experimental Neurology, 292: 46-55, 2017);
headaches including migraine (He et al., Journal of Neuroinflammation, 16: 78, 2019);
chronic kidney disease (Granata et al., PLoS One, 10(3): e0122272, 2015);
Sjogren's syndrome (Vakrakou et al., Journal of Autoimmunity, 91: 23-33, 2018);
graft-versus-host disease (Takahashi et al., Scientific Reports, 7: 13097, 2017);
sickle cell disease (Vogel et al., Blood, 130 (Suppl 1): 2234, 2017); and
colitis and inflammatory bowel diseases including ulcerative colitis and Crohn's disease (Braddock et al., Nat Rev Drug Disc, 3: 1-10, 2004; Neudecker et al., J Exp Med, 214(6): 1737-52, 2017; Wu et al., Mediators Inflamm, 2018: 3048532, 2018; and Lazaridis et al., Dig Dis Sci, 62(9): 2348-56, 2017), and sepsis (intestinal epithelial disruption) (Zhang et al., Dig Dis Sci, 63(1): 81-91, 2018).

Genetic ablation of NLRP3 has been shown to protect from HSD (high sugar diet), HFD (high fat diet) and HSFD-induced obesity (Pavillard et al., Oncotarget, 8(59): 99740-99756, 2017).

The NLRP3 inflammasome has been found to be activated in response to oxidative stress, sunburn (Hasegawa et al., Biochemical and Biophysical Research Communications, 477(3): 329-335, 2016), and UVB irradiation (Schroder et al., Science, 327: 296-300, 2010).

NLRP3 has also been shown to be involved in inflammatory hyperalgesia (Dolunay et al., Inflammation, 40: 366-386, 2017), wound healing (Ito et al., Exp Dermatol, 27(1): 80-86, 2018), burn healing (Chakraborty et al., Exp Dermatol, 27(1): 71-79, 2018), pain including allodynia, multiple sclerosis-associated neuropathic pain (Khan et al., Inflammopharmacology, 26(1): 77-86, 2018), chronic pelvic pain (Zhang et al., Prostate, 79(12): 1439-1449, 2019) and cancer-induced bone pain (Chen et al., Pharmacological Research, 147: 104339, 2019), and intra-amniotic inflammation/infection associated with preterm birth (Faro et al., Biol Reprod, 100(5): 1290-1305, 2019; and Gomez-Lopez et al., Biol Reprod, 100(5): 1306-1318, 2019).

The inflammasome, and NLRP3 specifically, has also been proposed as a target for modulation by various pathogens including bacterial pathogens such as Staphylococcus aureus, including methicillin-resistant Staphylococcus aureus (MRSA) (Cohen et al., Cell Reports, 22(9): 2431-2441, 2018; and Robinson et al., JCI Insight, 3(7): e97470, 2018), Mycobacterium tuberculosis (TB) (Subbarao et al., Scientific Reports, 10: 3709, 2020), Bacillus cereus (Mathur et al., Nat Microbiol, 4: 362-374, 2019), Salmonella typhimurium (Diamond et al., Sci Rep, 7(1): 6861, 2017), and group A streptococcus (LaRock et al., Science Immunology, 1(2): eaah3539, 2016); viruses such as DNA viruses (Amsler et al., Future Virol, 8(4): 357-370, 2013), influenza A virus (Coates et al., Front Immunol, 8: 782, 2017), chikungunya, Ross river virus, and alpha viruses (Chen et al., Nat Microbiol, 2(10): 1435-1445, 2017); fungal pathogens such as Candida albicans (Tucey et al., mSphere, 1(3), pii: e00074-16, 2016); and other pathogens such as T. gondii (Gov et al., J Immunol, 199(8): 2855-2864, 2017), helminth worms (Alhallaf et al., Cell Reports, 23(4): 1085-1098, 2018), leishmania (Novais et al., PLoS Pathogens, 13(2): e1006196, 2017), and plasmodium (Strangward et al., PNAS, 115(28): 7404-7409, 2018). NLRP3 has been shown to be required for the efficient control of viral, bacterial, fungal, and helminth pathogen infections (Strowig et al., Nature, 481: 278-286, 2012). NLRP3 activity has also been associated with increased susceptibility to viral infection such as by the human immunodeficiency virus (HIV) (Pontillo et al., J Aquir Immune Defic Syndr, 54(3): 236-240, 2010). An increased risk for early mortality amongst patients co-infected with HIV and Mycobacterium tuberculosis (TB) has also been associated with NLRP3 activity (Ravimohan et al., Open Forum Infectious Diseases, 5(5): of y075, 2018).

NLRP3 has been implicated in the pathogenesis of many cancers (Menu et al., Clinical and Experimental Immunology, 166: 1-15, 2011; and Masters, Clin Immunol, 147(3): 223-228, 2013). For example, several previous studies have suggested a role for IL-1β in cancer invasiveness, growth and metastasis, and inhibition of IL-1β with canakinumab has been shown to reduce the incidence of lung cancer and total cancer mortality in a randomised, double-blind, placebo-controlled trial (Ridker et al., Lancet, S0140-6736(17)32247-X, 2017). Inhibition of the NLRP3 inflammasome or IL-1β has also been shown to inhibit the proliferation and migration of lung cancer cells in vitro (Wang et al., Oncol Rep, 35(4): 2053-64, 2016), and NLRP3 has been shown to suppress NK cell-mediated control of carcinogenesis and metastases (Chow et al., Cancer Res, 72(22): 5721-32, 2012). A role for the NLRP3 inflammasome has been suggested in myelodysplastic syndromes (Basiorka et al., Blood, 128(25): 2960-2975, 2016) and also in the carcinogenesis of various other cancers including glioma (Li et al., Am J Cancer Res, 5(1): 442-449, 2015), colon cancer (Allen et al., J Exp Med, 207(5): 1045-56, 2010), melanoma (Dunn et al., Cancer Lett, 314(1): 24-33, 2012), breast cancer (Guo et al., Scientific Reports, 6: 36107, 2016), inflammation-induced tumours (Allen et al., J Exp Med, 207(5): 1045-56, 2010; and Hu et al., PNAS, 107(50): 21635-40, 2010), multiple myeloma (Li et al., Hematology, 21(3): 144-51, 2016), and squamous cell carcinoma of the head and neck (Huang et al., J Exp Clin Cancer Res, 36(1): 116, 2017; and Chen et al., Cellular and Molecular Life Sciences, 75: 2045-2058, 2018). Activation of the NLRP3 inflammasome has also been shown to mediate chemoresistance of tumour cells to 5-fluorouracil (Feng et al., J Exp Clin Cancer Res, 36(1): 81, 2017), and activation of the NLRP3 inflammasome in peripheral nerves contributes to chemotherapy-induced neuropathic pain (Jia et al., Mol Pain, 13: 1-11, 2017).

Accordingly, any of the diseases, disorders or conditions listed above may be treated or prevented in accordance with the fifth, sixth, seventh, eighth, ninth or tenth aspect of the present invention. Particular examples of diseases, disorders or conditions which may be responsive to NLRP3 inhibition and which may be treated or prevented in accordance with the fifth, sixth, seventh, eighth, ninth or tenth aspect of the present invention include:

(i) inflammation, including inflammation occurring as a result of an inflammatory disorder, e.g. an autoinflammatory disease, inflammation occurring as a symptom of a non-inflammatory disorder, inflammation occurring as a result of infection, or inflammation secondary to trauma, injury or autoimmunity;

(ii) auto-immune diseases such as acute disseminated encephalitis, Addison's disease, ankylosing spondylitis, antiphospholipid antibody syndrome (APS), anti-synthetase syndrome, aplastic anemia, autoimmune adrenalitis, autoimmune hepatitis, autoimmune oophoritis, autoimmune polyglandular failure, autoimmune thyroiditis, Coeliac disease including paediatric Coeliac disease, Crohn's disease, type 1 diabetes (T1D), Goodpasture's syndrome, Graves' disease, Guillain-Barre syndrome (GBS), Hashimoto's disease, idiopathic thrombocytopenic purpura, Kawasaki's disease, lupus erythematosus including systemic lupus erythematosus (SLE), multiple sclerosis (MS) including primary progressive multiple sclerosis (PPMS), secondary progressive multiple sclerosis (SPMS) and relapsing remitting multiple sclerosis (RRMS), myasthenia gravis, opsoclonus myoclonus syndrome (OMS), optic neuritis, Ord's thyroiditis, pemphigus, pernicious anaemia, polyarthritis, primary biliary cirrhosis, rheumatoid arthritis (RA), psoriatic arthritis, juvenile idiopathic arthritis or Still's disease, refractory gouty arthritis, Reiter's syndrome, Sjögren's syndrome, systemic sclerosis a systemic connective tissue disorder, Takayasu's arteritis, temporal arteritis, warm autoimmune hemolytic anemia, Wegener's granulomatosis, alopecia universalis, Behcet's disease, Chagas' disease, dysautonomia, endometriosis, hidradenitis suppurativa (HS), interstitial cystitis, neuromyotonia, psoriasis, sarcoidosis, scleroderma, ulcerative colitis, Schnitzler's syndrome, macrophage activation syndrome, Blau syndrome, vitiligo or vulvodynia;

(iii) cancer including lung cancer, pancreatic cancer, gastric cancer, myelodysplastic syndrome, leukaemia including acute lymphocytic leukaemia (ALL) and acute myeloid leukaemia (AML), adrenal cancer, anal cancer, basal and squamous cell skin cancer, squamous cell carcinoma of the head and neck, bile duct cancer, bladder cancer, bone cancer, brain and spinal cord tumours, breast cancer, cervical cancer, chronic lymphocytic leukaemia (CLL), chronic myeloid leukaemia (CML), chronic myelomonocytic leukaemia (CMML), colorectal cancer, endometrial cancer, oesophagus cancer, Ewing family of tumours, eye cancer, gallbladder cancer, gastrointestinal carcinoid tumours, gastrointestinal stromal tumour (GIST), gestational trophoblastic disease, glioma, Hodgkin lymphoma, Kaposi sarcoma, kidney cancer, laryngeal and hypopharyngeal cancer, liver cancer, lung carcinoid tumour, lymphoma including cutaneous T cell lymphoma, malignant mesothelioma, melanoma skin cancer, Merkel cell skin cancer, multiple myeloma, nasal cavity and paranasal sinuses cancer, nasopharyngeal cancer, neuroblastoma, non-Hodgkin lymphoma, non-small cell lung cancer, oral cavity and oropharyngeal cancer, osteosarcoma, ovarian cancer, penile cancer, pituitary tumours, prostate cancer, retinoblastoma, rhabdomyosarcoma, salivary gland cancer, skin cancer, small cell lung cancer, small intestine cancer, soft tissue sarcoma, stomach cancer, testicular cancer, thymus cancer, thyroid cancer including anaplastic thyroid cancer, uterine sarcoma, vaginal cancer, vulvar cancer, Waldenstrom macroglobulinemia, and Wilms tumour;

(iv) infections including viral infections (e.g. from influenza virus, human immunodeficiency virus (HIV), alphavirus (such as Chikungunya and Ross River virus), flaviviruses (such as Dengue virus and Zika virus), herpes viruses (such as Epstein Barr virus, cytomegalovirus, Varicella-zoster virus, and KSHV), poxviruses (such as vaccinia virus (Modified vaccinia virus Ankara) and Myxoma virus), adenoviruses (such as Adenovirus 5), or papillomavirus), bacterial infections (e.g. from Staphylococcus aureus (including MRSA), Helicobacter pylori, Bacillus anthracis, Bacillus cereus, Bordatella pertussis, Burkholderia pseudomallei, Corynebacterium diptheriae, Clostridium tetani, Clostridium botulinum, Streptococcus pneumoniae, Streptococcus pyogenes, Listeria monocytogenes, Hemophilus influenzae, Pasteurella multicida, Shigella dysenteriae, Mycobacterium tuberculosis, Mycobacterium leprae, Mycoplasma pneumoniae, Mycoplasma hominis, Neisseria meningitidis, Neisseria gonorrhoeae, Rickettsia rickettsii, Legionella pneumophila, Klebsiella pneumoniae, Pseudomonas aeruginosa, Propionibacterium acnes, Treponema pallidum, Chlamydia trachomatis, Vibrio cholerae, Salmonella typhimurium, Salmonella typhi, Borrelia burgdorferi, Uropathogenic Escherichia coli (UPEC) or Yersinia pestis), fungal infections (e.g. from Candida or Aspergillus species), protozoan infections (e.g. from Plasmodium, Babesia, Giardia, Entamoeba, Leishmania or Trypanosomes), helminth infections (e.g. from schistosoma, roundworms, tapeworms or flukes), prion infections, and co-infections with any of the aforementioned (e.g. with HIV and Mycobacterium tuberculosis);

(v) central nervous system diseases such as Parkinson's disease, Alzheimer's disease, dementia, motor neuron disease, Huntington's disease, cerebral malaria, brain injury from pneumococcal meningitis, intracranial aneurysms, intracerebral haemorrhages, sepsis-associated encephalopathy, perioperative neurocognitive disorder, postoperative cognitive dysfunction, early brain injury, traumatic brain injury, cerebral ischemia-reperfusion injury, stroke, general anesthesia neuroinflammation and amyotrophic lateral sclerosis;

(vi) metabolic diseases such as type 2 diabetes (T2D), atherosclerosis, obesity, gout, and pseudo-gout;

(vii) cardiovascular diseases such as hypertension, ischaemia, reperfusion injury including post-MI ischemic reperfusion injury, stroke including ischemic stroke, transient ischemic attack, myocardial infarction including recurrent myocardial infarction, heart failure including congestive heart failure and heart failure with preserved ejection fraction, cardiac hypertrophy and fibrosis, embolism, aneurysms including abdominal aortic aneurysm, metabolism induced cardiac injury, and pericarditis including Dressler's syndrome;

(viii) respiratory diseases including chronic obstructive pulmonary disorder (COPD), asthma such as allergic asthma, eosinophilic asthma, and steroid-resistant asthma, asbestosis, silicosis, volcanic ash induced inflammation, nanoparticle induced inflammation, cystic fibrosis and idiopathic pulmonary fibrosis;

(ix) liver diseases including non-alcoholic fatty liver disease (NAFLD) and non-alcoholic steatohepatitis (NASH) including advanced fibrosis stages F3 and F4, alcoholic fatty liver disease (AFLD), alcoholic steatohepatitis (ASH), ischemia reperfusion injury of the liver, fulminant hepatitis, liver fibrosis, and liver failure including acute liver failure;

(x) renal diseases including chronic kidney disease, oxalate nephropathy, nephrocalcinosis, glomerulonephritis, diabetic nephropathy, obesity related glomerulopathy, kidney fibrosis including chronic crystal nephropathy, acute renal failure, acute kidney injury, and renal hypertension;

(xi) ocular diseases including those of the ocular epithelium, age-related macular degeneration (AMD) (dry and wet), Sjögren's syndrome, uveitis, corneal infection, diabetic retinopathy, optic nerve damage, dry eye, and glaucoma;

(xii) skin diseases including dermatitis such as contact dermatitis and atopic dermatitis, contact hypersensitivity, psoriasis, sunburn, skin lesions, hidradenitis suppurativa (HS), other cyst-causing skin diseases, pyoderma gangrenosum, and acne vulgaris including acne conglobata;

(xiii) lymphatic conditions such as lymphangitis and Castleman's disease;

(xiv) psychological disorders such as depression and psychological stress;

(xv) graft versus host disease;

(xvi) pain such as pelvic pain, hyperalgesia, allodynia including mechanical allodynia, neuropathic pain including multiple sclerosis-associated neuropathic pain, and cancer-induced bone pain;

(xvii) conditions associated with diabetes including diabetic encephalopathy, diabetic retinopathy, diabetic nephropathy, diabetic vascular endothelial dysfunction, and diabetic hypoadiponectinemia;

(xviii) conditions associated with arthritis including arthritic fever;

(xix) headache including cluster headaches, idiopathic intracranial hypertension, migraine, low pressure headaches (e.g. post-lumbar puncture), Short-Lasting Unilateral Neuralgiform Headache With Conjunctival Injection and Tearing (SUNCT), and tension-type headaches;

(xx) wounds and burns, including skin wounds and skin burns; and

(xxi) any disease where an individual has been determined to carry a germline or somatic non-silent mutation in NLRP3.

In one embodiment, the disease, disorder or condition is selected from:

(i) inflammation;

(ii) an auto-immune disease;

(iii) cancer;

(iv) an infection;

(v) a central nervous system disease;

(vi) a metabolic disease;

(vii) a cardiovascular disease;

(viii) a respiratory disease;

(ix) a liver disease;

(x) a renal disease;

(xi) an ocular disease;

(xii) a skin disease;

(xiii) a lymphatic condition;

(xiv) a psychological disorder;

(xv) graft versus host disease;

(xvi) allodynia;

(xvii) a condition associated with diabetes; and

(xviii) any disease where an individual has been determined to carry a germline or somatic non-silent mutation in NLRP3.

In another embodiment, the disease, disorder or condition is selected from:

(i) cancer;

(ii) an infection;

(iii) a central nervous system disease;

(iv) a cardiovascular disease;

(v) a liver disease;

(vi) an ocular disease; or

(vii) a skin disease.

More typically, the disease, disorder or condition is selected from:

(i) cancer;

(ii) an infection;

(iii) a central nervous system disease; or

(iv) a cardiovascular disease.

In one embodiment, the disease, disorder or condition is selected from:

(i) acne conglobata;

(ii) atopic dermatitis;

(iii) Alzheimer's disease;

(iv) amyotrophic lateral sclerosis;

(v) age-related macular degeneration (AMD);

(vi) anaplastic thyroid cancer;

(vii) cryopyrin-associated periodic syndromes (CAPS);

(viii) contact dermatitis;

(ix) cystic fibrosis;

(x) congestive heart failure;

(xi) chronic kidney disease;

(xii) Crohn's disease;

(xiii) familial cold autoinflammatory syndrome (FCAS);

(xiv) Huntington's disease;

(xv) heart failure;

(xvi) heart failure with preserved ejection fraction;

(xvii) ischemic reperfusion injury;

(xviii) juvenile idiopathic arthritis;

(xix) myocardial infarction;

(xx) macrophage activation syndrome;

(xxi) myelodysplastic syndrome;

(xxii) multiple myeloma;

(xxiii) motor neuron disease;

(xxiv) multiple sclerosis;

(xxv) Muckle-Wells syndrome;

(xxvi) non-alcoholic steatohepatitis (NASH);

(xxvii) neonatal-onset multisystem inflammatory disease (NOMID);

(xxviii) Parkinson's disease;

(xxix) sickle cell disease;

(xxx) systemic juvenile idiopathic arthritis;

(xxxi) systemic lupus erythematosus;

(xxxii) traumatic brain injury;

(xxxiii) transient ischemic attack;

(xxxiv) ulcerative colitis; or

(xxxv) Valosin Containing Protein disease.

In another embodiment of the fifth, sixth, seventh, eighth, ninth or tenth aspect of the present invention, the treatment or prevention comprises a reduction in susceptibility to viral infection. For instance, the treatment or prevention may comprise a reduction in susceptibility to HIV infection.

In a further typical embodiment of the invention, the disease, disorder or condition is inflammation. Examples of inflammation that may be treated or prevented in accordance with the fifth, sixth, seventh, eighth, ninth or tenth aspect of the present invention include inflammatory responses occurring in connection with, or as a result of:

(i) a skin condition such as contact hypersensitivity, bullous pemphigoid, sunburn, psoriasis, atopical dermatitis, contact dermatitis, allergic contact dermatitis, seborrhoetic dermatitis, lichen planus, scleroderma, pemphigus, epidermolysis bullosa, urticaria, erythemas, or alopecia;

(ii) a joint condition such as osteoarthritis, systemic juvenile idiopathic arthritis, adult-onset Still's disease, relapsing polychondritis, rheumatoid arthritis, juvenile chronic arthritis, gout, or a seronegative spondyloarthropathy (e.g. ankylosing spondylitis, psoriatic arthritis or Reiter's disease);

(iii) a muscular condition such as polymyositis or myasthenia gravis; (iv) a gastrointestinal tract condition such as inflammatory bowel disease (including Crohn's disease and ulcerative colitis), colitis, gastric ulcer, Coeliac disease, proctitis, pancreatitis, eosinopilic gastro-enteritis, mastocytosis, antiphospholipid syndrome, or a food-related allergy which may have effects remote from the gut (e.g., migraine, rhinitis or eczema);

(v) a respiratory system condition such as chronic obstructive pulmonary disease (COPD), asthma (including eosinophilic, bronchial, allergic, intrinsic, extrinsic or dust asthma, and particularly chronic or inveterate asthma, such as late asthma and airways hyper-responsiveness), bronchitis, rhinitis (including acute rhinitis, allergic rhinitis, atrophic rhinitis, chronic rhinitis, rhinitis caseosa, hypertrophic rhinitis, rhinitis pumlenta, rhinitis sicca, rhinitis medicamentosa, membranous rhinitis, seasonal rhinitis e.g. hay fever, and vasomotor rhinitis), sinusitis, idiopathic pulmonary fibrosis (IPF), sarcoidosis, farmer's lung, silicosis, asbestosis, volcanic ash induced inflammation, adult respiratory distress syndrome, hypersensitivity pneumonitis, or idiopathic interstitial pneumonia;

(vi) a vascular condition such as atherosclerosis, Behcet's disease, vasculitides, or Wegener's granulomatosis;

(vii) an autoimmune condition such as systemic lupus erythematosus, Sjögren's syndrome, systemic sclerosis, Hashimoto's thyroiditis, type I diabetes, idiopathic thrombocytopenia purpura, or Graves disease;

(viii) an ocular condition such as uveitis, allergic conjunctivitis, or vernal conjunctivitis;

(ix) a nervous condition such as multiple sclerosis or encephalomyelitis;

(x) an infection or infection-related condition, such as Acquired Immunodeficiency Syndrome (AIDS), acute or chronic bacterial infection, acute or chronic parasitic infection, acute or chronic viral infection, acute or chronic fungal infection, meningitis, hepatitis (A, B or C, or other viral hepatitis), peritonitis, pneumonia, epiglottitis, malaria, dengue hemorrhagic fever, leishmaniasis, streptococcal myositis, Mycobacterium tuberculosis (including Mycobacterium tuberculosis and HIV co-infection), Mycobacterium avium intracellulare, Pneumocystis carinii pneumonia, orchitis/epidydimitis, legionella, Lyme disease, influenza A, Epstein-Barr virus infection, viral encephalitis/aseptic meningitis, or pelvic inflammatory disease; (xi) a renal condition such as mesangial proliferative glomerulonephritis, nephrotic syndrome, nephritis, glomerular nephritis, obesity related glomerulopathy, acute renal failure, acute kidney injury, uremia, nephritic syndrome, kidney fibrosis including chronic crystal nephropathy, or renal hypertension;

(xii) a lymphatic condition such as Castleman's disease;

(xiii) a condition of, or involving, the immune system, such as hyper IgE syndrome, lepromatous leprosy, familial hemophagocytic lymphohistiocytosis, or graft versus host disease;

(xiv) a hepatic condition such as chronic active hepatitis, non-alcoholic steatohepatitis (NASH), alcohol-induced hepatitis, non-alcoholic fatty liver disease (NAFLD), alcoholic fatty liver disease (AFLD), alcoholic steatohepatitis (ASH), primary biliary cirrhosis, fulminant hepatitis, liver fibrosis, or liver failure; (xv) a cancer, including those cancers listed above;

(xvi) a burn, wound, trauma, haemorrhage or stroke;

(xvii) radiation exposure;

(xviii) a metabolic disease such as type 2 diabetes (T2D), atherosclerosis, obesity, gout or pseudo-gout; and/or

(xix) pain such as inflammatory hyperalgesia, pelvic pain, allodynia, neuropathic pain, or cancer-induced bone pain.

In one embodiment of the fifth, sixth, seventh, eighth, ninth or tenth aspect of the present invention, the disease, disorder or condition is an autoinflammatory disease such as cryopyrin-associated periodic syndromes (CAPS), Muckle-Wells syndrome (MWS), familial cold autoinflammatory syndrome (FCAS), familial Mediterranean fever (FMF), neonatal onset multisystem inflammatory disease (NOMID), Tumour Necrosis Factor (TNF) Receptor-Associated Periodic Syndrome (TRAPS), hyperimmunoglobulinemia D and periodic fever syndrome (HIDS), deficiency of interleukin 1 receptor antagonist (DIRA), Majeed syndrome, pyogenic arthritis, pyoderma gangrenosum and acne syndrome (PAPA), adult-onset Still's disease (AOSD), haploinsufficiency of A20 (HA20), pediatric granulomatous arthritis (PGA), PLCG2-associated antibody deficiency and immune dysregulation (PLAID), PLCG2-associated autoinflammatory, antibody deficiency and immune dysregulation (APLAID), or sideroblastic anaemia with B-cell immunodeficiency, periodic fevers and developmental delay (SIFD).

Examples of diseases, disorders or conditions which may be responsive to NLRP3 inhibition and which may be treated or prevented in accordance with the fifth, sixth, seventh, eighth, ninth or tenth aspect of the present invention are listed above. Some of these diseases, disorders or conditions are substantially or entirely mediated by NLRP3 inflammasome activity, and NLRP3-induced IL-1β and/or IL-18. As a result, such diseases, disorders or conditions may be particularly responsive to NLRP3 inhibition and may be particularly suitable for treatment or prevention in accordance with the fifth, sixth, seventh, eighth, ninth or tenth aspect of the present invention. Examples of such diseases, disorders or conditions include cryopyrin-associated periodic syndromes (CAPS), Muckle-Wells syndrome (MWS), familial cold autoinflammatory syndrome (FCAS), neonatal onset multisystem inflammatory disease (NOMID), familial Mediterranean fever (FMF), pyogenic arthritis, pyoderma gangrenosum and acne syndrome (PAPA), hyperimmunoglobulinemia D and periodic fever syndrome (HIDS), Tumour Necrosis Factor (TNF) Receptor-Associated Periodic Syndrome (TRAPS), systemic juvenile idiopathic arthritis, adult-onset Still's disease (AOSD), relapsing polychondritis, Schnitzler's syndrome, Sweet's syndrome, Behcet's disease, anti-synthetase syndrome, deficiency of interleukin 1 receptor antagonist (DIRA), and haploinsufficiency of A20 (HA20).

Moreover, some of the diseases, disorders or conditions mentioned above arise due to mutations in NLRP3, in particular, resulting in increased NLRP3 activity. As a result, such diseases, disorders or conditions may be particularly responsive to NLRP3 inhibition and may be particularly suitable for treatment or prevention in accordance with the fifth, sixth, seventh, eighth, ninth or tenth aspect of the present invention. Examples of such diseases, disorders or conditions include cryopyrin-associated periodic syndromes (CAPS), Muckle-Wells syndrome (MWS), familial cold autoinflammatory syndrome (FCAS), and neonatal onset multisystem inflammatory disease (NOMID).

An eleventh aspect of the invention provides a method of inhibiting NLRP3, the method comprising the use of a compound of the first or second aspect of the invention, or a pharmaceutically acceptable salt, solvate or prodrug of the third aspect of the invention, or a pharmaceutical composition of the fourth aspect of the invention, to inhibit NLRP3.

In one embodiment of the eleventh aspect of the present invention, the method comprises the use of a compound of the first or second aspect of the invention, or a pharmaceutically acceptable salt, solvate or prodrug of the third aspect of the invention, or a pharmaceutical composition of the fourth aspect of the invention, in combination with one or more further active agents.

In one embodiment of the eleventh aspect of the present invention, the method is performed ex vivo or in vitro, for example in order to analyse the effect on cells of NLRP3 inhibition.

In another embodiment of the eleventh aspect of the present invention, the method is performed in vivo. For example, the method may comprise the step of administering an effective amount of a compound of the first or second aspect, or a pharmaceutically acceptable salt, solvate or prodrug of the third aspect, or a pharmaceutical composition of the fourth aspect, to thereby inhibit NLRP3. In one embodiment, the method further comprises the step of co-administering an effective amount of one or more further active agents. Typically, the administration is to a subject in need thereof.

Alternately, the method of the eleventh aspect of the invention may be a method of inhibiting NLRP3 in a non-human animal subject, the method comprising the steps of administering the compound, salt, solvate, prodrug or pharmaceutical composition to the non-human animal subject and optionally subsequently mutilating or sacrificing the non-human animal subject. Typically, such a method further comprises the step of analysing one or more tissue or fluid samples from the optionally mutilated or sacrificed non-human animal subject. In one embodiment, the method further comprises the step of co-administering an effective amount of one or more further active agents.

A twelfth aspect of the invention provides a compound of the first or second aspect of the invention, or a pharmaceutically acceptable salt, solvate or prodrug of the third aspect of the invention, or a pharmaceutical composition of the fourth aspect of the invention, for use in the inhibition of NLRP3. Typically, the use comprises the administration of the compound, salt, solvate, prodrug or pharmaceutical composition to a subject. In one embodiment, the compound, salt, solvate, prodrug or pharmaceutical composition is co-administered with one or more further active agents.

A thirteenth aspect of the invention provides the use of a compound of the first or second aspect of the invention, or a pharmaceutically effective salt, solvate or prodrug of the third aspect of the invention, in the manufacture of a medicament for the inhibition of NLRP3. Typically, the inhibition comprises the administration of the compound, salt, solvate, prodrug or medicament to a subject. In one embodiment, the compound, salt, solvate, prodrug or medicament is co-administered with one or more further active agents.

In any embodiment of any of the fifth to thirteenth aspects of the present invention that comprises the use or co-administration of one or more further active agents, the one or more further active agents may comprise for example one, two or three different further active agents.

The one or more further active agents may be used or administered prior to, simultaneously with, sequentially with or subsequent to each other and/or to the compound of the first or second aspect of the invention, the pharmaceutically acceptable salt, solvate or prodrug of the third aspect of the invention, or the pharmaceutical composition of the fourth aspect of the invention. Where the one or more further active agents are administered simultaneously with the compound of the first or second aspect of the invention, or the pharmaceutically acceptable salt, solvate or prodrug of the third aspect of the invention, a pharmaceutical composition of the fourth aspect of the invention may be administered wherein the pharmaceutical composition additionally comprises the one or more further active agents.

In one embodiment of any of the fifth to thirteenth aspects of the present invention that comprises the use or co-administration of one or more further active agents, the one or more further active agents are selected from:

(i) chemotherapeutic agents;

(ii) antibodies;

(iii) alkylating agents;

(iv) anti-metabolites;

(v) anti-angiogenic agents;

(vi) plant alkaloids and/or terpenoids;

(vii) topoisomerase inhibitors;

(viii) mTOR inhibitors;

(ix) stilbenoids;

(x) STING agonists;

(xi) cancer vaccines;

(xii) immunomodulatory agents;

(xiii) antibiotics;

(xiv) anti-fungal agents;

(xv) anti-helminthic agents; and/or

(xvi) other active agents.

It will be appreciated that these general embodiments defined according to broad categories of active agents are not mutually exclusive. In this regard any particular active agent may be categorized according to more than one of the above general embodiments. A non-limiting example is urelumab which is an antibody that is an immunomodulatory agent for the treatment of cancer.

As will be understood, where the further active agent is a small chemical entity, any reference to a specific small chemical entity below is to be understood to encompass all salt, hydrate, solvate, polymorphic and prodrug forms of the specific small chemical entity. Similarly, where the further active agent is a biologic such as a monoclonal antibody, any reference to a specific biologic below is to be understood to encompass all biosimilars thereof.

In some embodiments, the one or more chemotherapeutic agents are selected from abiraterone acetate, altretamine, amsacrine, anhydrovinblastine, auristatin, azacitidine, 5-azacytidine, azathioprine, adriamycin, bexarotene, bicalutamide, BMS 184476, bleomycin, bortezomib, N,N-dimethyl-L-valyl-L-valyl-N-methyl-L-valyl-L-prolyl-L-proline-t-butylamide, cisplatin, carboplatin, carboplatin cyclophosphamide, chlorambucil, cachectin, cemadotin, cyclophosphamide, carmustine, cladribine, cryptophycin, cytarabine, docetaxel, doxetaxel, doxorubicin, dacarbazine (DTIC), dactinomycin, daunorubicin, decitabine, dolastatin, etoposide, etoposide phosphate, enzalutamide (MDV3100), 5-fluorouracil, fludarabine, flutamide, gemcitabine, hydroxyurea and hydroxyureataxanes, idarubicin, ifosfamide, irinotecan, ixazomib, lenalidomide, lenalidomide-dexamethasone, leucovorin, lonidamine, lomustine (CCNU), larotaxel (RPR109881), mechlorethamine, mercaptopurine, methotrexate, mitomycin C, mitoxantrone, melphalan, mivobulin, 3′,4′-didehydro-4′-deoxy-8′-norvin-caleukoblastine, nilutamide, oxaliplatin, onapristone, prednimustine, procarbazine, paclitaxel, platinum-containing anti-cancer agents, 2,3,4,5,6-pentafluoro-N-(3-fluoro-4-methoxyphenyl)benzene sulfonamide, prednimustine, revlimid, rhizoxin, sertenef, streptozocin, stramustine phosphate, tretinoin, tasonermin, taxol, topotecan, tamoxifen, teniposide, taxane, tegafur/uracil, thalidomide, vincristine, vinblastine, vinorelbine, vindesine, vindesine sulfate, and/or vinflunine.

Alternatively or in addition, the one or more chemotherapeutic agents may be selected from CD59 complement fragment, fibronectin fragment, gro-beta (CXCL2), heparinases, heparin hexasaccharide fragment, human chorionic gonadotropin (hCG), Type I interferon ligands such as interferon alpha and interferon beta, Type I interferon mimetics, Type II interferon ligands such as interferon gamma, Type II interferon mimetics, interferon inducible protein (IP-10), kringle 5 (plasminogen fragment), metalloproteinase inhibitors (TIMPs), 2-methoxyestradiol, placental ribonuclease inhibitor, plasminogen activator inhibitor, platelet factor-4 (PF4), prolactin 16 kD fragment, proliferin-related protein (PRP), various retinoids, tetrahydrocortisol-S, thrombospondin-1 (TSP-1), transforming growth factor-beta (TGF-β), vasculostatin, vasostatin (calreticulin fragment), cytokines (including interleukins, such as interleukin-1, interleukin-2, interleukin-5, interleukin-10, interleukin-12, and interleukin-33), interleukin-1 ligands and mimetics (such as rilonacept, anakinra, and anakinra-dexamethasone), interleukin-2 ligands and mimetics, interleukin-5 ligands and mimetics, interleukin-10 ligands and mimetics, interleukin-12 ligands and mimetics, and/or interleukin-33 ligands and mimetics.

In some embodiments, the one or more antibodies may comprise one or more monoclonal antibodies. In some embodiments, the one or more antibodies are anti-TNFα and/or anti-IL-6 antibodies, in particular anti-TNFα and/or anti-IL-6 monoclonal antibodies. In some embodiments, the one or more antibodies are selected from abatacept, abciximab, adalimumab, alemtuzumab, atezolizumab, atlizumab, avelumab, basiliximab, belimumab, benralizumab, bevacizumab, bretuximab vedotin, brodalumab, canakinumab, cetuximab, ceertolizumab pegol, daclizumab, denosumab, dupilumab, durvalumab, eculizumab, efalizumab, elotuzumab, gemtuzumab, golimumab, guselkumab, ibritumomab tiuxetan, infliximab, ipilimumab, ixekizumab, mepolizumab, muromonab-CD3, natalizumab, nivolumab, ofatumumab, omalizumab, palivizumab, panitumuab, pembrolizumab, ranibizumab, reslizumab, risankizumab, rituximab, sarilumab, secukinumab, siltuximab, tildrakizumab, tocilizumab, tositumomab, trastuzumab, and/or ustekinumab.

In some embodiments, the one or more alkylating agents may comprise an agent capable of alkylating nucleophilic functional groups under conditions present in cells, including, for example, cancer cells. In some embodiments, the one or more alkylating agents are selected from cisplatin, carboplatin, mechlorethamine, cyclophosphamide, chlorambucil, ifosfamide and/or oxaliplatin. In some embodiments, the alkylating agent may function by impairing cell function by forming covalent bonds with amino, carboxyl, sulfhydryl, and/or phosphate groups in biologically important molecules. In some embodiments, the alkylating agent may function by modifying a cell's DNA.

In some embodiments, the one or more anti-metabolites may comprise an agent capable of affecting or preventing RNA or DNA synthesis. In some embodiments, the one or more anti-metabolites are selected from azathioprine and/or mercaptopurine.

In some embodiments, the one or more anti-angiogenic agents are selected from thalidomide, lenalidomide, endostatin, angiogenin inhibitors, angioarrestin, angiostatin (plasminogen fragment), basement-membrane collagen-derived anti-angiogenic factors (tumstatin, canstatin, or arrestin), anti-angiogenic antithrombin III, and/or cartilage-derived inhibitor (CDI).

In some embodiments, the one or more plant alkaloids and/or terpenoids may prevent microtubule function. In some embodiments, the one or more plant alkaloids and/or terpenoids are selected from a vinca alkaloid, a podophyllotoxin and/or a taxane. In some embodiments, the one or more vinca alkaloids may be derived from the Madagascar periwinkle, Catharanthus roseus (formerly known as Vinca rosea), and may be selected from vincristine, vinblastine, vinorelbine and/or vindesine. In some embodiments, the one or more taxanes are selected from taxol, paclitaxel, docetaxel and/or ortataxel. In some embodiments, the one or more podophyllotoxins are selected from an etoposide and/or teniposide.

In some embodiments, the one or more topoisomerase inhibitors are selected from a type I topoisomerase inhibitor and/or a type II topoisomerase inhibitor, and may interfere with transcription and/or replication of DNA by interfering with DNA supercoiling. In some embodiments, the one or more type I topoisomerase inhibitors may comprise a camptothecin, which may be selected from exatecan, irinotecan, lurtotecan, topotecan, BNP 1350, CKD 602, DB 67 (AR67) and/or ST 1481. In some embodiments, the one or more type II topoisomerase inhibitors may comprise an epipodophyllotoxin, which may be selected from an amsacrine, etoposid, etoposide phosphate and/or teniposide.

In some embodiments, the one or more mTOR (mammalian target of rapamycin, also known as the mechanistic target of rapamycin) inhibitors are selected from rapamycin, everolimus, temsirolimus and/or deforolimus.

In some embodiments, the one or more stilbenoids are selected from resveratrol, piceatannol, pinosylvin, pterostilbene, alpha-viniferin, ampelopsin A, ampelopsin E, diptoindonesin C, diptoindonesin F, epsilon-vinferin, flexuosol A, gnetin H, hemsleyanol D, hopeaphenol, trans-diptoindonesin B, astringin, piceid and/or diptoindonesin A.

In some embodiments, the one or more STING (Stimulator of interferon genes, also known as transmembrane protein (TMEM) 173) agonists may comprise cyclic di-nucleotides (CDNs), such as c-di-AMP, c-di-GMP, and cGAMP, and/or modified cyclic di-nucleotides that may include one or more of the following modification features: 2′-O/3′-O linkage, phosphorothioate linkage, adenine and/or guanine analogue, and/or 2′-OH modification (e.g. protection of the 2′-OH with a methyl group or replacement of the 2′-OH by —F or —N₃). In some embodiments, the one or more STING agonists are selected from BMS-986301, MK-1454, ADU-S100, a diABZI, 3′3′-cGAMP, and/or 2′3′-cGAMP.

In some embodiments, the one or more cancer vaccines are selected from an HPV vaccine, a hepatitis B vaccine, Oncophage, and/or Provenge.

In some embodiments, the one or more immunomodulatory agents may comprise an immune checkpoint inhibitor. The immune checkpoint inhibitor may target an immune checkpoint receptor, or combination of receptors comprising, for example, CTLA-4, PD-1, PD-L1, PD-L2, T cell immunoglobulin and mucin 3 (TIM3 or HAVCR2), galectin 9, phosphatidylserine, lymphocyte activation gene 3 protein (LAG3), MHC class I, MHC class II, 4-1BB, 4-1BBL, OX40, OX40L, GITR, GITRL, CD27, CD70, TNFRSF25, TL1A, CD40, CD40L, HVEM, LIGHT, BTLA, CD160, CD80, CD244, CD48, ICOS, ICOSL, B7-H3, B7-H4, VISTA, TMIGD2, HHLA2, TMIGD2, a butyrophilin (including BTNL2), a Siglec family member, TIGIT, PVR, a killer-cell immunoglobulin-like receptor, an ILT, a leukocyte immunoglobulin-like receptor, NKG2D, NKG2A, MICA, MICB, CD28, CD86, SIRPA, CD47, VEGF, neuropilin, CD30, CD39, CD73, CXCR4, and/or CXCL12.

In some embodiments, the immune checkpoint inhibitor is selected from urelumab, PF-05082566, MEDI6469, TRX518, varlilumab, CP-870893, pembrolizumab (PD1), nivolumab (PD1), atezolizumab (formerly MPDL3280A) (PD-L1), MEDI4736 (PD-L1), avelumab (PD-L1), PDR001 (PD1), BMS-986016, MGA271, lirilumab, IPH2201, emactuzumab, INCB024360, galunisertib, ulocuplumab, BKT140, bavituximab, CC-90002, bevacizumab, and/or MNRP1685A.

In some embodiments, the one or more immunomodulatory agents may comprise a complement pathway modulator. Complement pathway modulators modulate the complement activation pathway. Complement pathway modulators may act to block action of the C3 and/or C3a and/or C3aR1 receptor, or may act to block action of the C5 and/or C5a and/or C5aR1 receptor. In some embodiments, the complement pathway modulator is a C5 complement pathway modulator and may be selected from eculizumab, ravulizumab (ALXN1210), ABP959, RA101495, tesidolumab (LFG316), zimura, crovalimab (RO7112689), pozelimab (REGN3918), GNR-045, SOBI005, and/or coversin. In some embodiments, the complement pathway modulator is a C5a complement pathway modulator and may be selected from cemdisiran (ALN-CC5), IFX-1, IFX-2, IFX-3, and/or olendalizumab (ALXN1007). In some embodiments, the complement pathway modulator is a C5aR1 complement pathway modulator and may be selected from ALS-205, MOR-210/TJ210, DF2593A, DF3016A, DF2593A, avacopan (CCX168), and/or IPH5401.

In some embodiments, the one or more immunomodulatory agents may comprise an anti-TNFα agent. In some embodiments, the anti-TNFα agent may be an antibody or an antigen-binding fragment thereof, a fusion protein, a soluble TNFα receptor (e.g. a soluble TNFR1 or soluble TNFR2), an inhibitory nucleic acid, or a small molecule TNFα antagonist. In some embodiments, the inhibitory nucleic acid may be a ribozyme, a small hairpin RNA, a small interfering RNA, an antisense nucleic acid, or an aptamer. In some embodiments, the anti-TNFα agent is selected from adalimumab, certolizumab pegol, etanercept, golimumab, infliximab, CDP571, and biosimilars thereof (such as adalimumab-adbm, adalimumab-adaz, adalimumab-atto, etanercept-szzs, infliximab-abda and infliximab-dyyb).

In some embodiments, the one or more immunomodulatory agents may comprise azithromycin, clarithromycin, erythromycin, levofloxacin and/or roxithromycin.

In some embodiments, the one or more antibiotics are selected from amikacin, gentamicin, kanamycin, neomycin, netilmicin, tobramycin, paromomycin, streptomycin, spectinomycin, geldanamycin, herbimycin, rifaximin, loracarbef, ertapenem, doripenem, imipenem, cilastatin, meropenem, cefadroxil, cefazolin, cefalotin, cefalothin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftaroline fosamil, ceftobiprole, teicoplanin, vancomycin, telavancin, dalbavancin, oritavancin, clindamycin, lincomycin, daptomycin, azithromycin, clarithromycin, dirithromycin, erythromycin, roxithromycin, troleandomycin, telithromycin, spiramycin, aztreonam, furazolidone, nitrofurantoin, linezolid, posizolid, radezolid, torezolid, amoxicillin, ampicillin, azlocillin, carbenicillin, cloxacillin, dicloxacillin, flucloxacillin, mezlocillin, methicillin, nafcillin, oxacillin, penicillin G, penicillin V, piperacillin, temocillin, ticarcillin, calvulanate, ampicillin, subbactam, tazobactam, ticarcillin, clavulanate, bacitracin, colistin, polymyxin B, ciprofloxacin, enoxacin, gatifloxacin, gemifloxacin, levofloxacin, lomefloxacin, moxifloxacin, nalidixic acid, norfloxacin, ofloxacin, trovafloxacin, grepafloxacin, sparfloxacin, temafloxacin, mafenide, sulfacetamide, sulfadiazine, silver sulfadiazine, sulfadimethoxine, sulfamethoxazole, sulfanamide, sulfasalazine, sulfisoxazole, trimethoprim-sulfamethoxazole, sulfonamideochrysoidine, demeclocycline, minocycline, oytetracycline, tetracycline, clofazimine, dapsone, dapreomycin, cycloserine, ethambutol, ethionamide, isoniazid, pyrazinamide, rifampicin, rifabutin, rifapentine, streptomycin, arsphenamine, chloramphenicol, fosfomycin, fusidic acid, metronidazole, mupirocin, platensimycin, quinupristin, dalopristin, thiamphenicol, tigecycyline, tinidazole, trimethoprim, and/or teixobactin.

In some embodiments, the one or more antibiotics may comprise one or more cytotoxic antibiotics. In some embodiments, the one or more cytotoxic antibiotics are selected from an actinomycin, an anthracenedione, an anthracycline, thalidomide, dichloroacetic acid, nicotinic acid, 2-deoxyglucose, and/or chlofazimine. In some embodiments, the one or more actinomycins are selected from actinomycin D, bacitracin, colistin (polymyxin E) and/or polymyxin B. In some embodiments, the one or more antracenediones are selected from mitoxantrone and/or pixantrone. In some embodiments, the one or more anthracyclines are selected from bleomycin, doxorubicin (Adriamycin), daunorubicin (daunomycin), epirubicin, idarubicin, mitomycin, plicamycin and/or valrubicin.

In some embodiments, the one or more anti-fungal agents are selected from bifonazole, butoconazole, clotrimazole, econazole, ketoconazole, luliconazole, miconazole, omoconazole, oxiconazole, sertaconazole, sulconazole, tioconazole, albaconazole, efinaconazole, epoziconazole, fluconazole, isavuconazole, itraconazole, posaconazole, propiconazole, ravusconazole, terconazole, voriconazole, abafungin, amorolfin, butenafine, naftifine, terbinafine, anidulafungin, caspofungin, micafungin, benzoic acid, ciclopirox, flucytosine, 5-fluorocytosine, griseofulvin, haloprogin, tolnaflate, undecylenic acid, and/or balsam of Peru.

In some embodiments, the one or more anti-helminthic agents are selected from benzimidazoles (including albendazole, mebendazole, thiabendazole, fenbendazole, triclabendazole, and flubendazole), abamectin, diethylcarbamazine, ivermectin, suramin, pyrantel pamoate, levamisole, salicylanilides (including niclosamide and oxyclozanide), and/or nitazoxanide.

In some embodiments, other active agents are selected from growth inhibitory agents; anti-inflammatory agents (including non-steroidal anti-inflammatory agents; small molecule anti-inflammatory agents (such as colchicine); and anti-inflammatory biologics that target for example TNF, IL-5, IL-6, IL-17 or IL-33); JAK inhibitors; phosphodiesterase inhibitors; CAR T therapies; anti-psoriatic agents (including anthralin and its derivatives); vitamins and vitamin-derivatives (including retinoinds, and VDR receptor ligands); steroids; corticosteroids; glucocorticoids (such as dexamethasone, prednisone and triamcinolone acetonide); ion channel blockers (including potassium channel blockers); immune system regulators (including cyclosporin, FK 506, and glucocorticoids); lutenizing hormone releasing hormone agonists (such as leuprolidine, goserelin, triptorelin, histrelin, bicalutamide, flutamide and/or nilutamide); hormones (including estrogen); and/or uric acid lowering agents (such as allopurinol).

Unless stated otherwise, in any of the fifth to thirteenth aspects of the invention, the subject may be any human or other animal. Typically, the subject is a mammal, more typically a human or a domesticated mammal such as a cow, pig, lamb, sheep, goat, horse, cat, dog, rabbit, mouse etc. Most typically, the subject is a human.

Any of the medicaments employed in the present invention can be administered by oral, parenteral (including intravenous, subcutaneous, intramuscular, intradermal, intratracheal, intraperitoneal, intraarticular, intracranial and epidural), airway (aerosol), rectal, vaginal, ocular or topical (including transdermal, buccal, mucosal, sublingual and topical ocular) administration.

Typically, the mode of administration selected is that most appropriate to the disorder, disease or condition to be treated or prevented. Where one or more further active agents are administered, the mode of administration may be the same as or different to the mode of administration of the compound, salt, solvate, prodrug or pharmaceutical composition of the invention.

For oral administration, the compounds, salts, solvates or prodrugs of the present invention will generally be provided in the form of tablets, capsules, hard or soft gelatine capsules, caplets, troches or lozenges, as a powder or granules, or as an aqueous solution, suspension or dispersion.

Tablets for oral use may include the active ingredient mixed with pharmaceutically acceptable excipients such as inert diluents, disintegrating agents, binding agents, lubricating agents, sweetening agents, flavouring agents, colouring agents and preservatives. Suitable inert diluents include sodium and calcium carbonate, sodium and calcium phosphate, and lactose. Corn starch and alginic acid are suitable disintegrating agents. Binding agents may include starch and gelatine. The lubricating agent, if present, may be magnesium stearate, stearic acid or tale. If desired, the tablets may be coated with a material, such as glyceryl monostearate or glyceryl distearate, to delay absorption in the gastrointestinal tract. Tablets may also be effervescent and/or dissolving tablets.

Capsules for oral use include hard gelatine capsules in which the active ingredient is mixed with a solid diluent, and soft gelatine capsules wherein the active ingredient is mixed with water or an oil such as peanut oil, liquid paraffin or olive oil.

Powders or granules for oral use may be provided in sachets or tubs. Aqueous solutions, suspensions or dispersions may be prepared by the addition of water to powders, granules or tablets.

Any form suitable for oral administration may optionally include sweetening agents such as sugar, flavouring agents, colouring agents and/or preservatives.

Formulations for rectal administration may be presented as a suppository with a suitable base comprising, for example, cocoa butter or a salicylate.

Formulations suitable for vaginal administration may be presented as pessaries, tampons, creams, gels, pastes, foams or spray formulations containing in addition to the active ingredient such carriers as are known in the art to be appropriate.

For parenteral use, the compounds, salts, solvates or prodrugs of the present invention will generally be provided in a sterile aqueous solution or suspension, buffered to an appropriate pH and isotonicity. Suitable aqueous vehicles include Ringer's solution and isotonic sodium chloride or glucose. Aqueous suspensions according to the invention may include suspending agents such as cellulose derivatives, sodium alginate, polyvinylpyrrolidone and gum tragacanth, and a wetting agent such as lecithin. Suitable preservatives for aqueous suspensions include ethyl and n-propyl p-hydroxybenzoate. The compounds of the invention may also be presented as liposome formulations.

For ocular administration, the compounds, salts, solvates or prodrugs of the invention will generally be provided in a form suitable for topical administration, e.g. as eye drops. Suitable forms may include ophthalmic solutions, gel-forming solutions, sterile powders for reconstitution, ophthalmic suspensions, ophthalmic ointments, ophthalmic emulsions, ophthalmic gels and ocular inserts. Alternatively, the compounds, salts, solvates or prodrugs of the invention may be provided in a form suitable for other types of ocular administration, for example as intraocular preparations (including as irrigating solutions, as intraocular, intravitreal or juxtascleral injection formulations, or as intravitreal implants), as packs or corneal shields, as intracameral, subconjunctival or retrobulbar injection formulations, or as iontophoresis formulations.

For transdermal and other topical administration, the compounds, salts, solvates or prodrugs of the invention will generally be provided in the form of ointments, cataplasms (poultices), pastes, powders, dressings, creams, plasters or patches.

Suitable suspensions and solutions can be used in inhalers for airway (aerosol) administration.

The dose of the compounds, salts, solvates or prodrugs of the present invention will, of course, vary with the disease, disorder or condition to be treated or prevented. In general, a suitable dose will be in the range of 0.01 to 500 mg per kilogram body weight of the recipient per day. The desired dose may be presented at an appropriate interval such as once every other day, once a day, twice a day, three times a day or four times a day. The desired dose may be administered in unit dosage form, for example, containing 1 mg to 50 g of active ingredient per unit dosage form.

For the avoidance of doubt, insofar as is practicable any embodiment of a given aspect of the present invention may occur in combination with any other embodiment of the same aspect of the present invention. In addition, insofar as is practicable it is to be understood that any preferred, typical or optional embodiment of any aspect of the present invention should also be considered as a preferred, typical or optional embodiment of any other aspect of the present invention.

### Examples—Compound Synthesis

All solvents, reagents and compounds were purchased and used without further purification unless stated otherwise.

### Abbreviations

2-MeTHF 2-methyltetrahydrofuran
Ac₂O acetic anhydride
AcOH acetic acid
app apparent
aq aqueous
B₂Pin₂ bis(pinacolato)diboron, also called 4,4,4′,4′,5,5,5′,5′-octamethyl-2,2′-bi(1,3,2-dioxaborolane)
Boc tert-butyloxycarbonyl
BOP (benzotriazol-1-yloxy)tris(dimethylamino)phosphonium hexafluorophosphate
br broad
CAN ceric ammonium nitrate
Cbz carboxybenzyl
CDI 1,1-carbonyl-diimidazole
conc concentrated
d doublet
DABCO 1,4-diazabicyclo[2.2.2]octane
DBU 1,8-diazabicyclo[5.4.0]undec-7-ene
DCE 1,2-dichloroethane, also called ethylene dichloride
DCM dichloromethane
dd double doublet
ddd double double doublet
DIAD diisopropyl azodicarboxylate
DIC N,N′-diisopropylcarbodiimide
DIPEA N,N-diisopropylethylamine, also called Hünig's base
DMA dimethylacetamide
DMAP 4-dimethylaminopyridine, also called N,N-dimethylpyridin-4-amine
DME dimethoxyethane
DMF N,N-dimethylformamide
DMPU N,N′-dimethyl-N,N′-propylene urea
DMSO dimethyl sulfoxide
EDC 1-ethyl-3-(3-dimethylaminopropyl)carbodiimide
eq or equiv equivalent
(ES⁺) electrospray ionization, positive mode
Et ethyl
EtOAc ethyl acetate
EtOH ethanol
Ex example
FC flash column chromatography on silica gel
h hour(s)
HATU 1-[bis(dimethylamino)methylene]-1H-1,2,3-triazolo[4,5-b]pyridinium 3-oxid hexafluorophosphate
HPLC high performance liquid chromatography
Hz hertz
Int intermediate
KOAc potassium acetate
KO^{t}Bu potassium tert-butoxide
LC liquid chromatography
m multiplet
m-CPBA 3-chloroperoxybenzoic acid
Me methyl
MeCN acetonitrile
MeOH methanol
(M+H)⁺ protonated molecular ion
MHz megahertz
min minute(s)
MS mass spectrometry
Ms mesyl, also called methanesulfonyl
MsCl mesyl chloride, also called methanesulfonyl chloride
MTBE methyl tert-butyl ether, also called tert-butyl methyl ether
m/z mass-to-charge ratio
NaO^{t}Bu sodium tert-butoxide
NBS 1-bromopyrrolidine-2,5-dione, also called N-bromosuccinimide
NCS 1-chloropyrrolidine-2,5-dione, also called N-chlorosuccinimide
NMP N-methylpyrrolidine
NMR nuclear magnetic resonance (spectroscopy)
p pentuplet
Pd₂(dba)₃ tris(dibenzylideneacetone) dipalladium(0)
PdCl₂(dppf) [1,1′-bis(diphenylphosphino)ferrocene] dichloropalladium(II), also called Pd(dppf)Cl₂
PE petroleum ether
Ph phenyl
PMB p-methoxybenzyl, also called 4-methoxybenzyl
prep-HPLC preparative high performance liquid chromatography
prep-TLC preparative thin layer chromatography
PTSA p-toluenesulfonic acid
q quartet
QPhos 1,2,3,4,5-pentaphenyl-1′-(di-tert-butylphosphino)ferrocene
RP reversed phase
RT room temperature
s singlet
sat saturated
SCX solid supported cation exchange (resin)
sept septuplet
SPhos-Pd-G3 (2-dicyclohexylphosphino-2′,6′-dimethoxybiphenyl) [2-(2′-amino-1,1′-biphenyl)]palladium(II) methanesulfonate
t triplet
T₃P propylphosphonic anhydride
TBME tert-butyl methyl ether, also called methyl tert-butyl ether
TEA triethylamine
Tf triflyl, also called trifluoromethanesulfonyl
TFA 2,2,2-trifluoroacetic acid
THF tetrahydrofuran
TLC thin layer chromatography
wt % weight percent or percent by weight
XantPhos 4,5-bis(diphenylphosphino)-9,9-dimethylxanthene
XPhos 2-dicyclohexylphosphino-2′,4′,6′-triisopropylbiphenyl
XPhos-Pd-G3 (2-dicyclohexylphosphino-2′,4′,6′-triisopropyl-1,1′-biphenyl)[2-(2′-amino-1.1′-biphenyl)]palladium(II) methanesulfonate

### EXPERIMENTAL METHODS

### Nuclear Magnetic Resonance

NMR spectra were recorded at 300, 400 or 500 MHz. Spectra were measured at 298 K, unless indicated otherwise, and were referenced relative to the solvent resonance. The chemical shifts are reported in parts per million. Spectra were recorded using one of the following machines:
a Bruker Avance III spectrometer at 400 MHz fitted with a BBO 5 mm liquid probe,
a Bruker 400 MHz spectrometer using ICON-NMR, under TopSpin program control,
a Bruker Avance III HD spectrometer at 500 MHz, equipped with a Bruker 5 mm SmartProbe™,
an Agilent VNMRS 300 instrument fitted with a 7.05 Tesla magnet from Oxford instruments, indirect detection probe and direct drive console including PFG module, or
an Agilent MercuryPlus 300 instrument fitted with a 7.05 Tesla magnet from Oxford instruments, 4 nuclei auto-switchable probe and Mercury plus console.

### LC-MS

LC-MS Methods: Using SHIMADZU LCMS-2020, Agilent 1200 LC/G1956A MSD and Agilent 1200G6110A, Agilent 1200 LC & Agilent 6110 MSD. Mobile Phase: A: 0.025% NH₃.H₂O in water (v/v); B: acetonitrile. Column: Kinetex EVO C18 2.1×30 mm, 5 μm.

### Preparative Reversed Phase HPLC General Methods

Acidic prep HPLC (x-y % MeCN in water): Waters X-Select CSH column C18, 5 m (19×50 mm), flow rate 28 mL min-1 eluting with a H₂O-MeCN gradient containing 0.1% v/v formic acid over 6.5 min using UV detection at 254 nm. Gradient information: 0.0-0.2 min, x % MeCN; 0.2-5.5 min, ramped from x % MeCN to y % MeCN; 5.5-5.6 min, ramped from y % MeCN to 95% MeCN; 5.6-6.5 min, held at 95% MeCN.

Acidic prep HPLC (x-y % MeOH in water): Waters X-Select CSH column C18, 5 m (19×50 mm), flow rate 28 mL min-1 eluting with a 10 mM aq formic acid-MeOH gradient over 7.5 min using UV detection at 254 nm. Gradient information: 0.0-1.5 min, x % MeOH; 1.5-6.8 min, ramped from x % MeOH to y % MeOH; 6.8-6.9 min, ramped from y % MeOH to 95% MeOH; 6.9-7.5 min, held at 95% MeOH.

Basic prep HPLC (x-y % MeCN in water): Waters X-Bridge Prep column C18, 5 μm (19×50 mm), flow rate 28 mL min-1 eluting with a 10 mM NH₄HCO₃-MeCN gradient over 6.5 min using UV detection at 254 nm. Gradient information: 0.0-0.2 min, x % MeCN; 0.2-5.5 min, ramped from x % MeCN to y % MeCN; 5.5-5.6 min, ramped from y % MeCN to 95% MeCN; 5.6-6.5 min, held at 95% MeCN.

### Synthesis of Intermediates

### Intermediate A1: 1-(1-hydroxy-2-methylpropan-2-yl)-N,N-bis(4-methoxybenzyl)-1H-pyrazole-3-sulfonamide

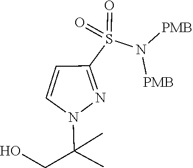

### Step A: lithium 1-(tetrahydro-2H-pyran-2-yl)-1H-pyrazole-5-sulfinate

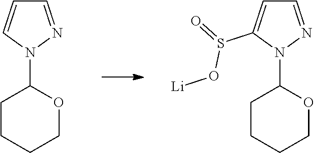

A solution of n-BuLi (100 mL, 250 mmol, 2.5 M in hexanes) was added slowly to a solution of 1-(tetrahydro-2H-pyran-2-yl)-1H-pyrazole (36.2 g, 238 mmol) in THF (500 mL), keeping the temperature below −65° C. The mixture was stirred for 1.5 h, then sulfur dioxide was bubbled through for 10 min. The mixture was allowed to warm to RT, the solvent evaporated and the residue triturated with MTBE (300 mL) and filtered. The solid was washed with MTBE and isohexane and dried to afford the crude title compound (54.89 g, 99%).

LCMS m/z 215 (M−Li)⁻ (ES⁻).

¹H NMR (DMSO-d₆) δ 7.26 (d, J=1.6 Hz, 1H), 6.10 (d, J=1.7 Hz, 1H), 5.99 (dd, J=10.0, 2.5 Hz, 1H), 3.92-3.87 (m, 1H), 3.56-3.49 (m, 1H), 2.25-2.15 (m, 1H), 2.00-1.91 (m, 1H), 1.75-1.69 (m, 1H), 1.66-1.46 (m, 3H).

### Step B: N,N-bis(4-methoxybenzyl)-1-(tetrahydro-2H-pyran-2-yl)-1H-pyrazole-5-sulfonamide

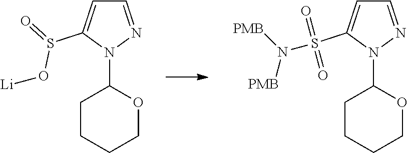

NCS (12.0 g, 90 mmol) was added to a suspension of lithium 1-(tetrahydro-2H-pyran-2-yl)-1H-pyrazole-5-sulfinate (20 g, 90 mmol) in DCM (250 mL) cooled in an ice bath. The mixture was stirred for 4 h, quenched with water (100 mL), and then partitioned between DCM (300 mL) and water (200 mL). The organic phase was washed with water (200 mL), dried (MgSO₄), filtered and evaporated to ˜50 mL. The solution was added to a mixture of bis(4-methoxybenzyl)amine (24 g, 93 mmol) and triethylamine (40 mL, 287 mmol) in DCM (300 mL) cooled in an ice bath. After stirring for 1 h, the mixture was warmed to RT, and then partitioned between DCM (300 mL) and water (250 mL). The organic layer was washed with water (250 mL), aq 1 M HCl (2×250 mL), water (250 mL), dried (MgSO₄), filtered, and evaporated to afford the crude title compound (41.02 g, 97%) as a brown oil.

LCMS m/z 494.2 (M+Na)⁺ (ES⁺).

### Step C: N,N-bis(4-methoxybenzyl)-1H-pyrazole-3-sulfonamide

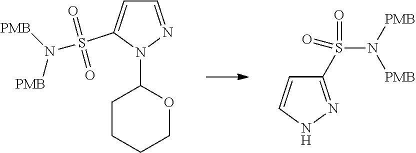

A mixture of N,N-bis(4-methoxybenzyl)-1-(tetrahydro-2H-pyran-2-yl)-1H-pyrazole-5-sulfonamide (41 g, 87 mmol) and aq 1 M HCl (30 mL) in THF (300 mL) and MeOH (50 mL) was stirred at RT for 18 h. The solvent was evaporated and the residue partitioned between EtOAc (400 mL) and aq 1 M HCl (200 mL). The organic layer was washed with 10% brine (200 mL), dried (MgSO₄), filtered and evaporated. The residue was triturated with MTBE, filtered and dried to afford the title compound (24.87 g, 69%) as an off white solid.

LCMS m/z 388 (M+H)⁺ (ES⁺); 386 (M−H)⁻ (ES⁻).

¹H NMR (CDCl₃) δ 7.88 (d, J=2.4 Hz, 1H), 7.06-7.02 (m, 4H), 6.79-6.75 (m, 4H), 6.63 (d, J=2.4 Hz, 1H), 4.31 (s, 4H), 3.78 (s, 6H). One exchangeable proton not observed.

### Step D: methyl 2-(3-(N,N-bis(4-methoxybenzyl)sulfamoyl)-1H-pyrazol-1-yl)-2-methylpropanoate

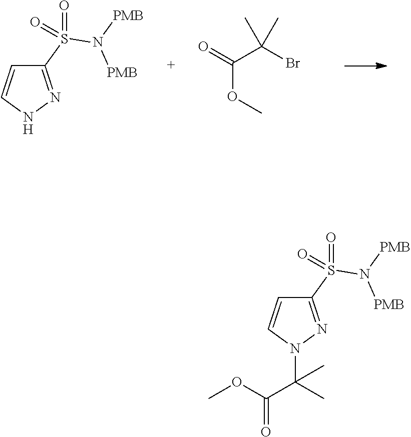

N,N-bis(4-Methoxybenzyl)-1H-pyrazole-3-sulfonamide (2.00 g, 5.16 mmol) and K₂CO₃ (2.14 g, 15-49 mmol) were suspended in DMF (30 mL). Methyl 2-bromo-2-methylpropanoate (1.00 mL, 7.74 mmol) was added and the mixture was heated to 80° C. overnight. The reaction mixture was cooled to RT, diluted with water (20 mL), poured onto brine (200 mL) and washed with MTBE (2×50 mL). The combined organic layers were dried (MgSO₄) and concentrated in vacuo. The crude product was purified by FC (0-70% EtOAc/isohexane) to afford the title compound (2.45 g, 94%) as a clear colourless oil.

LCMS m/z 510.6 (M+Na)⁺ (ES⁺).

¹H NMR (DMSO-d₆) δ 8.18 (d, J=2.5 Hz, 1H), 7.05-6.95 (m, 4H), 6.85-6.78 (m, 4H), 6.78 (d, J=2.5 Hz, 1H), 4.18 (s, 4H), 3.72 (s, 6H), 3.65 (s, 3H), 1.81 (s, 6H).

### Step E: 1-(1-hydroxy-2-methylpropan-2-yl)-N,N-bis(4-methoxybenzyl)-1H-pyrazole-3-sulfonamide

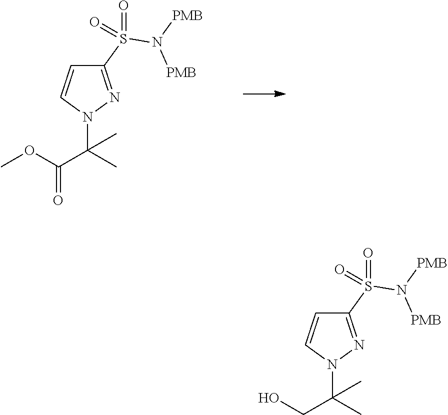

Methyl 2-(3-(N,N-bis(4-methoxybenzyl)sulfamoyl)-1H-pyrazol-1-yl)-2-methyl-propanoate (3.28 g, 6.73 mmol) was dissolved in THF (30 mL) and cooled to 0° C. LiAlH₄ (2 M in THF, 3.36 mL, 6.73 mmol) was added drop-wise and the reaction was stirred at RT for 16 h, quenched with slow addition of water (20 mL), diluted with brine (50 mL) and extracted with EtOAc (2×50 mL). The combined organic layers were dried (phase separator) and concentrated in vacuo to afford the title compound (3.43 g, 100%) as a white solid.

LCMS m/z 482.3 (M+Na)⁺ (ES⁺).

¹H NMR (DMSO-d₆) δ 8.00 (d, J=2.5 Hz, 1H), 7.04-6.98 (m, 4H), 6.84-6.80 (m, 4H), 6.69 (d, J=2.5 Hz, 1H), 5.14 (t, J=5.5 Hz, 1H), 4.20 (s, 4H), 3.72 (s, 6H), 3.61 (d, J=5.6 Hz, 2H), 1.50 (s, 6H).

### Intermediate A2: 1-(2-hydroxyethyl)-N,N-bis(4-methoxybenzyl)-1H-pyrazole-3-sulfonamide

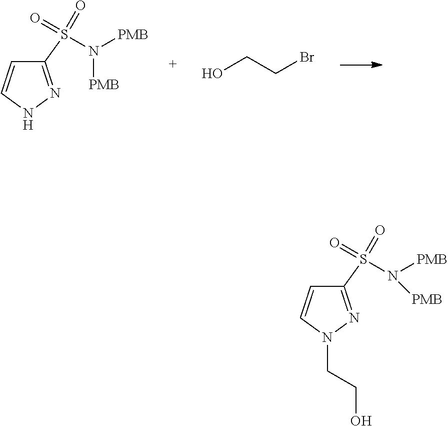

N,N-bis(4-methoxybenzyl)-1H-pyrazole-3-sulfonamide (Intermediate A1, Step C) (1 g, 2.58 mmol) and K₂CO₃ (0.892 g, 6.45 mmol) were suspended in MeCN (25 mL) under N₂. 2-Bromoethanol (0.2 mL, 2.84 mmol) was added and the mixture was heated to 50° C. for 4 h. After cooling to RT, water (50 mL) and EtOAc (75 mL) were added and the organic layer separated, dried (MgSO₄) and concentrated in vacuo. The crude product was purified by FC (0-100% EtOAc/isohexane) to afford the title compound (682.4 mg, 58%) as a thick colourless oil.

LCMS m/z 454.1 (M+Na)⁺ (ES⁺).

¹H NMR (DMSO-d₆) δ 7.93 (d, J=2.3 Hz, 1H), 7.04-6.98 (m, 4H), 6.85-6.79 (m, 4H), 6.71 (d, J=2.3 Hz, 1H), 5.01 (t, J=5.2 Hz, 1H), 4.27 (t, J=5.5 Hz, 2H), 4.20 (s, 4H), 3.78 (q, J=5.4 Hz, 2H), 3.72 (s, 6H).

### Intermediate A3: 5-((dimethylamino)methyl)-1-(5-hydroxypentyl)-N,N-bis(4-methoxybenzyl)-1H-pyrazole-3-sulfonamide

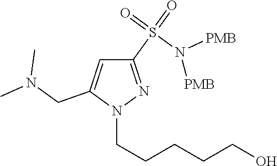

### Step A: 1-(5-hydroxypentyl)-N,N-bis(4-methoxybenzyl)-1H-pyrazole-3-sulfonamide

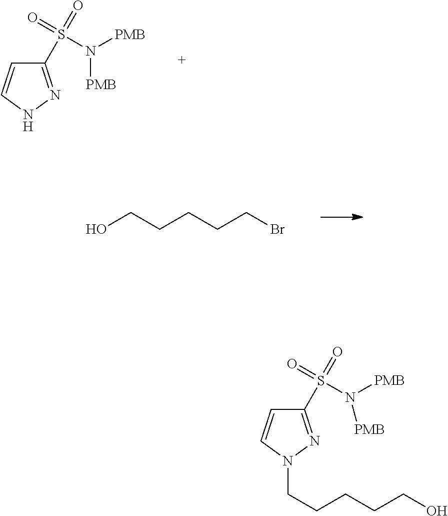

Prepared according to the general procedure of 1-(2-hydroxyethyl)-N,N-bis(4-methoxybenzyl)-1H-pyrazole-3-sulfonamide (Intermediate A2) to afford the title compound (1.13 g, 49%) as a thick colourless oil.

LCMS m/z 496.3 (M+Na)⁺ (ES⁺).

¹H NMR (DMSO-d₆) δ 7.97 (d, J=2.3 Hz, 1H), 7.08-6.94 (m, 4H), 6.90-6.75 (m, 4H), 6.71 (d, J=2.3 Hz, 1H), 4.39 (t, J=5.1 Hz, 1H), 4.25-4.15 (m, 6H), 3.72 (s, 6H), 3.44-3.34 (m, 2H), 1.80 (p, J=7.2 Hz, 2H), 1.51-1.38 (m, 2H), 1.32-1.21 (m, 2H).

### Step B: 5-((dimethylamino)methyl)-1-(5-hydroxypentyl)-N,N-bis(4-methoxybenzyl)-1H-pyrazole-3-sulfonamide

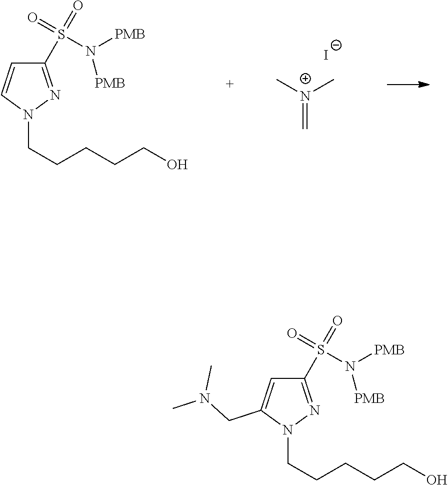

1-(5-Hydroxypentyl)-N,N-bis(4-methoxybenzyl)-1H-pyrazole-3-sulfonamide (1.25 g, 2.64 mmol) was dissolved in THF (50 mL) and cooled to −78° C. n-BuLi (2.5 M in hexanes, 2.64 mL, 6.60 mmol) was added immediately followed by N-methyl-N-methylenemethanaminium iodide (1.95 g, 10.54 mmol). The reaction was stirred for 1 h while allowing to warm to RT. The reaction was quenched with water (50 mL), extracted with MTBE (2×50 mL), dried (phase separator) and concentrated in vacuo. The resulting residue was dissolved in MeOH (100 mL) and stirred with SCX (7.5 g) for 30 min. The resin was washed with methanol (150 mL) and the desired product was eluted with 0.7 M ammonia in methanol (250 mL). The resulting solution was concentrated in vacuo to afford the title compound (416 mg, 29%) as a yellow oil.

¹H NMR (DMSO-d₆) δ 7.04-6.99 (m, 4H), 6.83-6.78 (m, 4H), 6.57 (s, 1H), 4.38 (t, J=5.1 Hz, 1H), 4.23-4.15 (m, 6H), 3.72 (s, 6H), 3.47 (s, 2H), 3.40-3.35 (m, 2H), 2.17 (s, 6H), 1.78 (p, J=7.5 Hz, 2H), 1.45 (p, J=6.7 Hz, 2H), 1.33-1.25 (m, 2H).

### Intermediate A4: 4-fluoro-1-(1-hydroxy-2-methylpropan-2-yl)-N,N-bis(4-methoxy-benzyl)-1H-pyrazole-3-sulfonamide

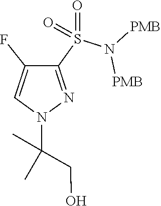

### Step A: 4-fluoro-1-(tetrahydro-2H-pyran-2-yl)-1H-pyrazole

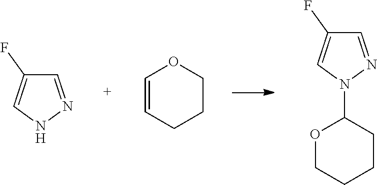 [SMILES: Fc1cn[nH]c1 ; C1=COCCC1 ; Fc1cnn(C2CCCCO2)c1]

A solution of 4-fluoro-1H-pyrazole (2 g, 23.24 mmol), 3,4-dihydro-2H-pyran (9 mL, 99 mmol) and TFA (0.40 mL, 5.19 mmol) in THF (25 mL) was heated to reflux overnight. The reaction was concentrated in vacuo and the crude product was purified by FC (0-50% EtOAc/isohexane) to afford the title compound (4-33 g, 93%) as a pale yellow oil.

¹H NMR (CDCl₃) δ 7.48 (d, J=4.7 Hz, 1H), 7.40 (d, J=4.3 Hz, 1H), 5.34-5.24 (m, 1H), 4.07-4.04 (m, 1H), 3.77-3.61 (m, 1H), 2.12-1.94 (m, 3H), 1.76-1.55 (m, 3H).

### Step B: lithium 4-fluoro-1-(tetrahydro-2H-pyran-2-yl)-1H-pyrazole-5-sulfinate

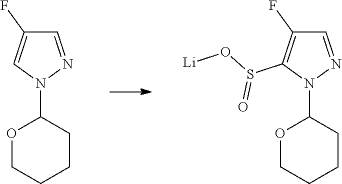

n-BuLi (2.5 M in THF) (5 mL, 12.50 mmol) was added slowly to a solution of 4-fluoro-1-(tetrahydro-2H-pyran-2-yl)-1H-pyrazole (2 g, 11.75 mmol) in THF (25 mL) keeping the temperature below −65° C. The mixture was stirred for 1.5 h then SO₂ was bubbled through for 10 min. The mixture was allowed to warm to RT, the solvent evaporated and the residue triturated with MTBE (50 mL) and filtered. The solid was washed with MTBE, isohexane and dried to afford the title compound (1.91 g, 64%) as a white solid.

¹H NMR (DMSO-d₆) δ 7.25 (d, J=4.6 Hz, 1H), 6.08 (dd, J=10.2, 2.5 Hz, 1H), 3.93-3.86 (m, 1H), 3.54-3.46 (m, 1H), 2.19-2.08 (m, 1H), 1.98-1.89 (m, 1H), 1.71-1.64 (m, 1H), 1.64-1.51 (m, 1H), 1.51-1.43 (m, 2H).

### Step C: 4-fluoro-N,N-bis(4-methoxybenzyl)-1-(tetrahydro-2H-pyran-2-yl)-1H-pyrazole-5-sulfonamide

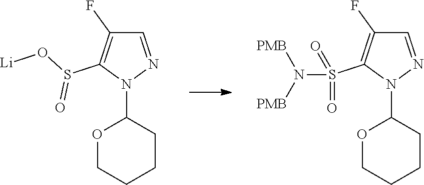

NCS (2.78 g, 20.82 mmol) was added to a suspension of lithium 4-fluoro-1-(tetrahydro-2H-pyran-2-yl)-1H-pyrazole-5-sulfinate (5.00 g, 20.82 mmol) in DCM (100 mL) cooled in an ice bath. The mixture was stirred for 18 h, quenched with water (10 mL) then partitioned between DCM (50 mL) and water (20 mL). The aqueous layer was extracted with DCM (2×100 mL) and the organic layers were dried (MgSO₄) and concentrated in vacuo to ˜100 mL. The solution was added to a mixture of bis(4-methoxybenzyl)amine (5.63 g, 21.86 mmol) and triethylamine (3.4 mL, 24.39 mmol) in DCM (30 mL) cooled in an ice bath. The mixture was allowed to warm to RT and stirred for 18 h, then partitioned between DCM (60 mL) and water (40 mL). The aqueous layer was extracted with DCM (2×30 mL) and the combined organic layers were dried (MgSO₄) and concentrated to dryness to afford a yellow oil. The crude product was purified by FC (0-50% EtOAc/isohexane) to afford the title compound (5.05 g, 40%) as a yellow crystalline solid.

LCMS m/z 512.1 (M+Na)⁺ (ES⁺).

¹H NMR (DMSO-d₆) δ 7.86 (d, J=4.5 Hz, 1H), 7.03-6.95 (m, 4H), 6.86-6.78 (m, 4H), 5.79 (dd, J=9.6, 2.6 Hz, 1H), 4.42 (d, J=15.4 Hz, 2H), 4.23 (d, J=15.5 Hz, 2H), 3.95-3.80 (m, 1H), 3.72 (s, 6H), 3.61-3.50 (m, 1H), 2.41-2.19 (m, 1H), 2.08-1.93 (m, 1H), 1.93-1.80 (m, 1H), 1.70-1.65 (m, 1H), 1.55-1.44 (m, 2H).

### Step D: 4-fluoro-N,N-bis(4-methoxybenzyl)-1H-pyrazole-3-sulfonamide

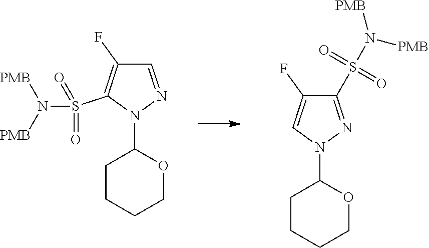

HCl (4 M in dioxane, 1 mL, 4.00 mmol) was added to a solution of 4-fluoro-N,N-bis(4-methoxybenzyl)-1-(tetrahydro-2H-pyran-2-yl)-1H-pyrazole-5-sulfonamide (4.25 g, 6.95 mmol) in DCM (50 mL). The mixture was heated at 40° C. for 3 days and concentrated in vacuo. The product was purified by FC (0-50% EtOAc/isohexane) to afford the title compound (3-54 g, quantitative yield) as a thick yellow oil.

LCMS m/z 512.2 (M+Na)⁺ (ES⁺).

¹H NMR (DMSO-d₆) δ 8.30 (d, J=4.6 Hz, 1H), 7.09-7.03 (m, 4H), 6.86-6.81 (m, 4H), 5.43 (dd, J=9.3, 2.5 Hz, 1H), 4.37-4.19 (m, 4H), 3.93-3.87 (m, 1H), 3.73 (s, 6H), 3.70-3.62 (m, 1H), 2.08-1.95 (m, 1H), 1.94-1.81 (m, 2H), 1.74-1.62 (m, 1H), 1.61-1.46 (m, 2H).

### Step E: 4-fluoro-N,N-bis(4-methoxybenzyl)-1H-pyrazole-3-sulfonamide

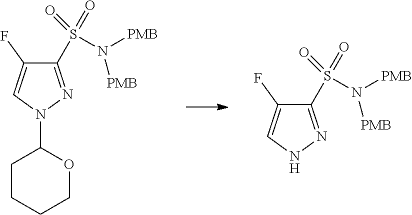

Concentrated HCl (10 mL, 120 mmol) was added to 4-fluoro-N,N-bis(4-methoxy-benzyl)-1-(tetrahydro-2H-pyran-2-yl)-1H-pyrazole-3-sulfonamide (3.50 g, 6.86 mmol) in MeOH (80 mL) at RT. The mixture was stirred at RT for 18 h. The methanol was removed in vacuo and the remaining aqueous suspension was quenched with sat aq NaHCO₃ drop-wise to pH 8. EtOAc (50 mL) was added and the organic layer was collected. The aqueous layer was extracted with EtOAc (50 mL) and the combined organic layers were concentrated in vacuo to afford a white solid which was triturated with MTBE (50 mL) to give a first crop of title compound (1.90 g). The filtrate was concentrated to dryness and purified by FC (0-100% EtOAc/isohexane). Both batches were combined to afford the title compound (2.59 g, 92%) as a white solid.

LCMS m/z 427.3 (M+Na)⁺ (ES⁺); 404.1 (M−H)⁻ (ES⁻).

¹H NMR (DMSO-d₆) δ 8.11-7.87 (m, 1H), 7.13-6.99 (m, 4H), 6.87-6.72 (m, 4H), 4.24 (s, 4H), 3.72 (s, 6H). One exchangeable proton not observed.

### Step F: methyl 2-(3-(N,N-bis(4-methoxybenzyl)sulfamoyl)-4-fluoro-1H-pyrazol-1-yl)-2-methylpropanoate

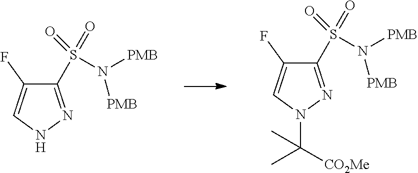

4-Fluoro-N,N-bis(4-methoxybenzyl)-1H-pyrazole-3-sulfonamide (1.00 g, 2.466 mmol) and K₂CO₃ (1.10 g, 7.96 mmol) were suspended in dry DMF (45 mL). Methyl 2-bromo-2-methylpropanoate (0.48 mL, 3.71 mmol) was added and the mixture was warmed to 80° C. for 3 h. The reaction mixture was cooled to RT, diluted with water (20 mL), poured onto brine (100 mL) and extracted with MTBE (2×50 mL). The combined organic layers were dried (MgSO₄), filtered and concentrated in vacuo. The crude product was purified by FC (0-100% EtOAc/isohexane) to afford the title compound (1.22 g, 92%) as a thick colourless oil.

LCMS m/z 527.7 (M+Na)⁺ (ES⁺).

¹H NMR (DMSO-d₆) δ 8.41 (d, J=4.5 Hz, 1H), 7.09-6.96 (m, 4H), 6.88-6.75 (m, 4H), 4.23 (s, 4H), 3.72 (s, 6H), 3.66 (s, 3H), 1.76 (s, 6H).

### Step G: 4-fluoro-1-(1-hydroxy-2-methylpropan-2-yl)-N,N-bis(4-methoxybenzyl)-1H-pyrazole-3-sulfonamide

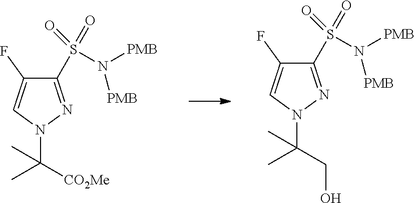

LiBH₄ (4 M solution in THF) (1.81 mL, 7.24 mmol) was added dropwise to a stirred solution of methyl 2-(3-(N,N-bis(4-methoxybenzyl)sulfamoyl)-4-fluoro-1H-pyrazol-1-yl)-2-methylpropanoate (1.22 g, 2.413 mmol) in THF (25 mL) at 0° C. The mixture was stirred for 17 h. The mixture was partitioned between water (20 mL) and EtOAc (50 mL). The organic layer was collected and the aqueous layer was extracted with EtOAc (2×20 mL). The combined organic layers were dried (MgSO₄), filtered and concentrated to dryness to afford the title compound (1.01 g, 83%) as a sticky colourless foam.

LCMS m/z 500.1 (M+Na)⁺ (ES⁺).

¹H NMR (DMSO-d₆) δ 8.19 (d, J=4.6 Hz, 1H), 7.10-7.00 (m, 4H), 6.87-6.78 (m, 4H), 5.18-5.09 (m, 1H), 4.24 (s, 4H), 3.72 (s, 6H), 3.55 (d, J=3.8 Hz, 2H), 1.44 (s, 6H).

### Intermediate A5: 5-(2-hydroxyethyl)-1-isopropyl-N,N-bis(4-methoxybenzyl)-1H-pyrazole-3-sulfonamide

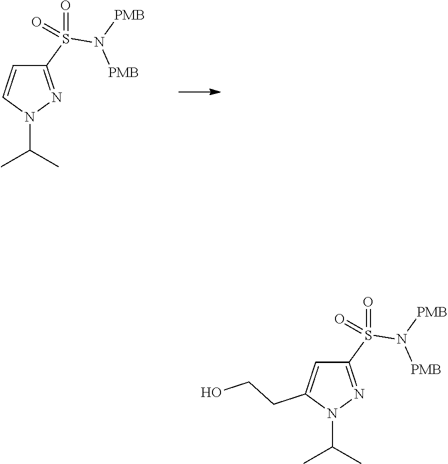

A solution of n-BuLi (2.5 M in hexanes) (2 mL, 5.00 mmol) was added dropwise to a stirred solution of 1-isopropyl-N,N-bis(4-methoxybenzyl)-1H-pyrazole-3-sulfonamide (2.067 g, 4.81 mmol) in THF (35 mL) at −78° C. The reaction was stirred for 1 h and oxirane (2.5 M in THF) (7.70 mL, 19.25 mmol) was added. The reaction mixture was left at −78° C. for 1 h. The reaction mixture was warmed up to RT and stirred for 48 h. The reaction was quenched with water (20 mL) and extracted with EtOAc (2×20 mL). The organic layer was separated, dried over MgSO4, filtered and concentrated in vacuo. The crude product was purified by FC (0-10% MeOH/DCM) to afford the title compound (1.33 g, 56%) as a yellow oil.

LCMS m/z 496.4 (M+Na)⁺ (ES⁺).

¹H NMR (DMSO-d₆) δ 7.06-6.97 (m, 4H), 6.85-6.78 (m, 4H), 6.51 (s, 1H), 4.87 (t, J=5.2 Hz, 1H), 4.68 (sept, J=6.6 Hz, 1H), 4.19 (s, 4H), 3.72 (s, 6H), 3.70-3.63 (m, 2H), 2.84 (t, J=6.5 Hz, 2H), 1.38 (d, J=6.6 Hz, 6H).

### Intermediate A6: 3-(2-hydroxyethyl)-N,N-bis(4-methoxybenzyl)benzene-sulfonamide

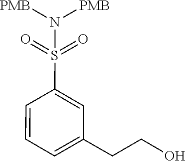

### Step A: 3-(2-hydroxyethyl)benzene-1-sulfonyl chloride

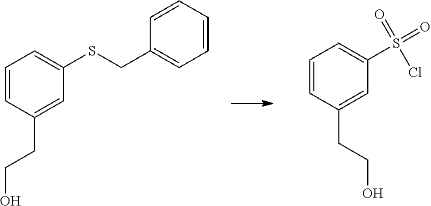 [SMILES: OCCc1cccc(SCc2ccccc2)c1 ; O=S(=O)(Cl)c1cccc(CCO)c1]

A solution of 2-(3-(benzylthio)phenyl)ethanol (1.21 g, 4.95 mmol) in MeCN (25 mL), AcOH (0.3 mL) and water (0.6 mL) was cooled to −10° C. 1,3-Dichloro-5,5-dimethyl-imidazolidine-2,4-dione (1.50 g, 7.61 mmol) was then added and the mixture was stirred at −10° C. for 4 h. The mixture was then partitioned between DCM (50 mL) and water (50 mL). The aqueous layer was extracted with DCM (100 mL) and the combined organic layers were dried (MgSO₄) and concentrated in vacuo to afford the crude title compound which was used without further purification in the next step.

### Step B: 3-(2-hydroxyethyl)-N,N-bis(4-methoxybenzyl)benzenesulfonamide

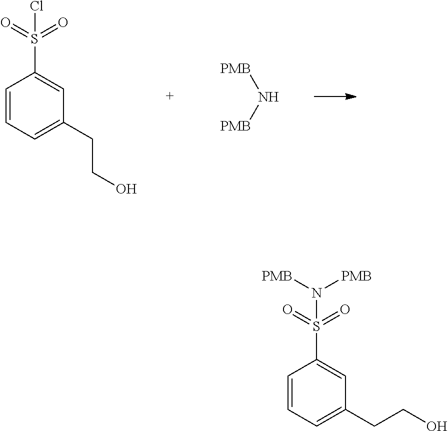

bis-(4-Methoxybenzyl)amine (1.30 g, 5.05 mmol) was added to a suspension of 3-(2-hydroxyethyl)benzene-1-sulfonyl chloride (1.09 g, 4.95 mmol) in DCM (25 mL) cooled in an ice bath, followed by Et₃N (1.5 mL, 10.76 mmol). The mixture was stirred for 17 h, quenched with water (20 mL) then partitioned between DCM (50 mL) and water (40 mL). The organic phase was dried (MgSO₄) and concentrated in vacuo. The crude was purified by FC (0-50% EtOAc/isohexane) to afford the title compound (1.40 g, 60% over 2 steps) as a white solid.

LCMS m/z 464.1 (M+Na)⁺ (ES⁺).

¹H NMR (DMSO-d₆) δ 7.72-7.61 (m, 2H), 7.57-7.44 (m, 2H), 7.02-6.93 (m, 4H), 6.83-6.75 (m, 4H), 4.69 (t, J=5.1 Hz, 1H), 4.18 (s, 4H), 3.71 (s, 6H), 3.63 (td, J=6.7, 5.0 Hz, 2H), 2.80 (t, J=6.7 Hz, 2H).

### Intermediate A7: 3-(2-hydroxyethyl)-5-(2-hydroxypropan-2-yl)-N,N-bis(4-methoxybenzyl)benzenesulfonamide

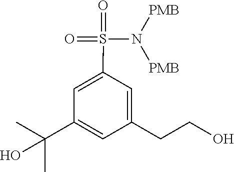

### Step A: methyl 3-(N,N-bis(4-methoxybenzyl)sulfamoyl)-5-bromobenzoate

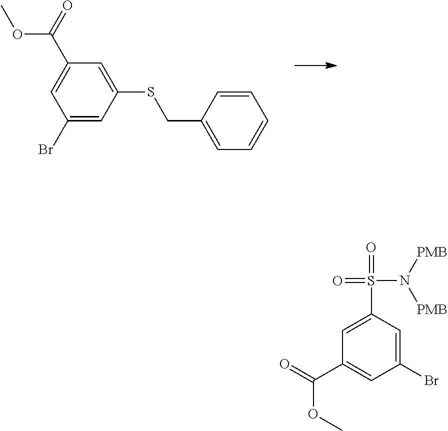

A solution of methyl 3-(benzylthio)-5-bromobenzoate (3.76 g, 11.15 mmol) in MeCN (54 mL), AcOH (0.7 mL) and water (1.4 mL) was cooled to −10° C. (ice/acetone bath). 1,3-dichloro-5,5-dimethylimidazolidine-2,4-dione (3.30 g, 16.72 mmol) was then added and the mixture was stirred at −10° C. for 4 h. The mixture was then partitioned between DCM (250 mL) and water (200 mL) and the organic layer was collected. The aqueous layer was extracted with DCM (100 mL) and the combined organic layers were dried (MgSO₄), filtered and concentrated in vacuo to give methyl 3-bromo-5-(chloro-sulfonyl)benzoate (3.50 g, 11.15 mmol) as a thick yellow oil. To a suspension of methyl 3-bromo-5-(chlorosulfonyl)benzoate (3.50 g, 11-15 mmol) in DCM (25 mL) was added bis(4-methoxybenzyl)amine (2.87 g, 11.15 mmol) whilst being cooled with an ice bath, followed by TEA (3 mL, 21.52 mmol). The mixture was stirred for 17 h, quenched with water (20 mL) then partitioned between DCM (50 mL) and water (40 mL). The organic phase was collected, dried (MgSO₄), filtered and concentrated in vacuo to give a brown oil. The brown oil was purified by FC (0-50% EtOAc/isohexane) to afford the title compound (2.77 g, 43%) as a white solid.

¹H NMR (DMSO-d₆) δ 8.21 (app t, J=1.7 Hz, 1H), 8.01 (app d, J=1.7 Hz, 2H), 7.19-7.02 (m, 4H), 6.87-6.71 (m, 4H), 4.32 (s, 4H), 3.90 (s, 3H), 3.71 (s, 6H).

### Step B: 3-bromo-5-(2-hydroxypropan-2-yl)-N,N-bis(4-methoxybenzyl)-benzene-sulfonamide

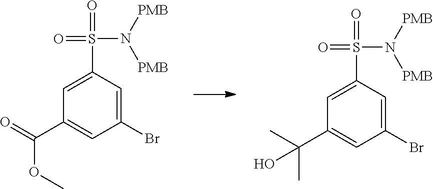

MeMgBr (3 M in Et₂O) (3.5 mL, 10.50 mmol) was added dropwise to a stirred solution of methyl 3-(N,N-bis(4-methoxybenzyl)sulfamoyl)-5-bromobenzoate (2.47 g, 4.25 mmol) in THF (2 mL) cooled to 0° C. The mixture was stirred at 0° C. for 1 h then left to warm to RT with stirring over 1 h. Additional MeMgBr (3 M in Et₂O) (3.5 mL, 10.50 mmol) was added and the mixture stirred for a further 1 h. The mixture was quenched with water (20 mL) and brine (50 mL) at 0° C. and extracted with EtOAc (2×100 mL). The combined organic layers were dried (MgSO₄), filtered and concentrated in vacuo to afford the title compound (2.50 g, 96%) as a colourless oil which crystallised to give a white solid.

¹H NMR (DMSO-d₆) δ 7.89 (t, J=1.7 Hz, 1H), 7.86 (t, J=1.7 Hz, 1H), 7.63 (t, J=1.7 Hz, 1H), 7.06-6.99 (m, 4H), 6.83-6.78 (m, 4H), 5.42 (s, 1H), 4.25 (s, 4H), 3.71 (s, 6H), 1.43 (s, 6H).

### Step C: tert-butyl 2-(3-(N,N-bis(4-methoxybenzyl)sulfamoyl)-5-(2-hydroxypropan-2-yl)phenyl)acetate

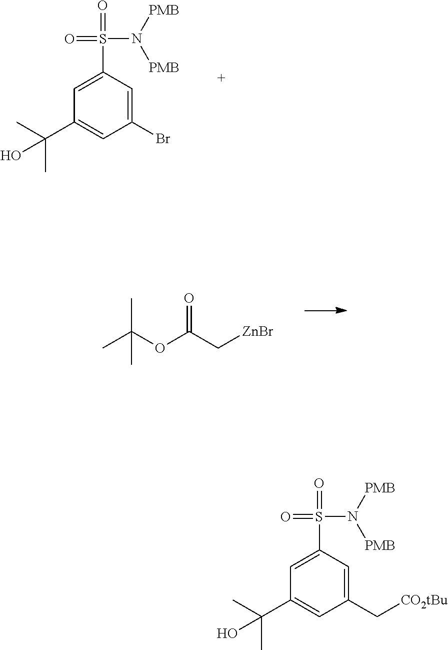

(2-(tert-Butoxy)-2-oxoethyl)zinc(II) bromide 0.44 M (29 mL, 12.76 mmol) was added to a solution of 3-bromo-5-(2-hydroxypropan-2-yl)-N,N-bis(4-methoxybenzyl)-benzenesulfonamide (2.50 g, 4.07 mmol), Pd₂(dba)₃ (0.186 g, 0.203 mmol) and QPhos (0.289 g, 0.407 mmol) in anhydrous THF (250 mL) and the reaction was stirred at 70° C. for 21 h. The reaction mixture was left to cool to RT and the mixture was quenched with water (50 mL) and the THF removed in vacuo. The red/grey emulsion obtained was diluted with EtOAc (100 mL) and filtered. The organic layer was collected and the aqueous layer was extracted with EtOAc (2×50 mL). The combined organic layers were dried (MgSO₄), filtered and concentrated in vacuo to give a red oil which was purified by FC (0-50% EtOAc/isohexane) to afford the title compound (2.10 g, 73%) as a red oil.

LCMS m/z 592.4 (M+Na)⁺ (ES⁺).

¹H NMR (DMSO-d₆) δ 7.81-7.77 (m, 1H), 7.65-7.61 (m, 1H), 7.59-7.57 (m, 1H), 6.99-6.93 (m, 4H), 6.81-6.75 (m, 4H), 5.29 (s, 1H), 4.17 (s, 4H), 3.70 (s, 6H), 3.69 (s, 2H), 1.42 (s, 6H), 1.39 (s, 9H).

### Step D: 3-(2-hydroxyethyl)-5-(2-hydroxypropan-2-yl)-N,N-bis(4-methoxybenzyl)-benzenesulfonamide

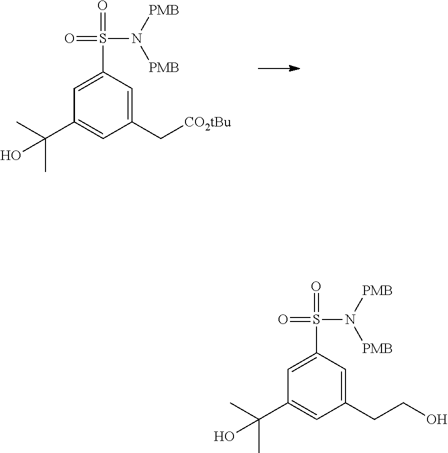

4 M LiBH₄ in THF (2.76 mL, 11.0 mmol) was added dropwise to a stirred solution of tert-butyl 2-(3-(N,N-bis(4-methoxybenzyl)sulfamoyl)-5-(2-hydroxypropan-2-yl)-phenyl)acetate (2.1 g, 3.69 mmol) in THF (50 mL) cooled to 0° C. The mixture was stirred for 2 h. Additional 4 M LiBH₄ in THF (2.76 mL, 11.06 mmol) was added and allowed to stir for 1 h. Additional 4 M LiBH₄ in THF (2.76 mL, 11.06 mmol) was added and the reaction was allowed to stir overnight. Additional 4 M LiBH₄ in THF (2.76 mL, 11.06 mmol) was added and the reaction allowed to stir for a further 16 h. The mixture was then partitioned between water (30 mL) and EtOAc (50 mL). The organic layer was collected and the aqueous layer was extracted with EtOAc (2×40 mL). The combined organic layers were dried (phase separator) and concentrated in vacuo to afford the title compound (1.8 g, 88%) as a dark red oil.

LCMS m/z 522.3 (M+Na)⁺ (ES⁺).

¹H NMR (DMSO-d₆) δ 7.76-7.73 (m, 1H), 7.61-7.58 (m, 1H), 7.51-7.48 (m, 1H), 7.01-6.96 (m, 4H), 6.82-6.76 (m, 4H), 5.24 (s, 1H), 4.69 (t, J=5.1 Hz, 1H), 4.18 (s, 4H) 3.71 (s, 6H), 3.66-3.59 (m, 2H), 2.80 (t, J=6.7 Hz, 2H), 1.43 (s, 6H).

### Intermediate A8: 4-fluoro-1-(2-hydroxyethyl)-N,N-bis(4-methoxybenzyl)-1H-pyrazole-3-sulfonamide

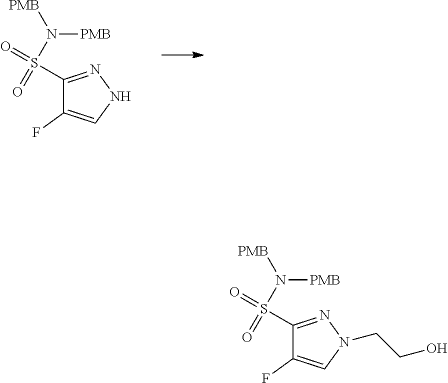

4-Fluoro-N,N-bis(4-methoxybenzyl)-1H-pyrazole-3-sulfonamide (Intermediate A4, Step E) (1.00 g, 2.466 mmol) and potassium carbonate (1.10 g, 7.96 mmol) were suspended in dry acetonitrile (10 mL) under a N₂ atmosphere. 2-bromoethanol (0.23 mL, 3.21 mmol) was added and the mixture was heated to 50° C. for 3 h. After cooling to RT, water (20 mL) and EtOAc (20 mL) were added. The organic phase was dried (MgSO₄), filtered and concentrated to dryness to give a pale yellow oil. The crude product was purified by FC (0-100% EtOAc/isohexane) to afford the title compound (0.88 g, 70%) as a thick colourless oil.

LCMS m/z 472.1 (M+Na)⁺ (ES⁺).

¹H NMR (DMSO-d₆) δ 8.11 (d, J=4.6 Hz, 1H), 7.06-6.99 (m, 4H), 6.85-6.80 (m, 4H), 5.03 (t, J=5.3 Hz, 1H), 4.23 (s, 4H), 4.17 (t, J=5.4 Hz, 2H), 3.75 (q, J=5.4 Hz, 2H), 3.72 (s, 6H).

### Intermediate A9: 1-(1-hydroxy-2-methylpropan-2-yl)-N,N-bis(4-methoxybenzyl)-1H-pyrazole-4-sulfonamide

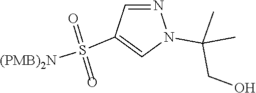

### Step A: N,N-bis(4-methoxybenzyl)-1H-pyrazole-4-sulfonamide

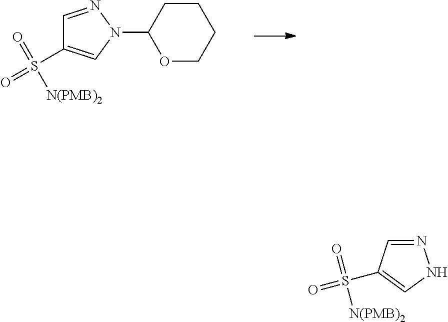

A solution of N,N-bis(4-methoxybenzyl)-1-(tetrahydro-2H-pyran-2-yl)-1H-pyrazole-4-sulfonamide (2.12 g, 4.50 mmol) in methanol (45 mL, 45.0 mmol) and dioxane (45 mL), to which was added conc aq HCl (7.50 mL, 4.50 mmol), was stirred at 50° C. for 3 h. The mixture was partitioned between EtOAc (150 mL) and water (100 mL), the phases separated and the aqueous further extracted with EtOAc (2×100 mL). The organic phases were combined, dried (MgSO₄), filtered and concentrated under reduced pressure to 1/10 of the volume and directly loaded onto the column for purification by FC (0-100% EtOAc/isohexane) to afford the title compound (1.50 g, 81%) as a white solid.

LCMS m/z 386.3 (M−H)⁻ (ES⁻).

¹H NMR (DMSO-d₆) δ 13.64 (s, 1H), 8.10 (s, 2H), 7.09-7.02 (m, 4H), 6.85-6.74 (m, 4H), 4.12 (s, 4H), 3.71 (s, 6H).

### Step B: methyl 2-(4-(N,N-bis(4-methoxybenzyl)sulfamoyl)-1H-pyrazol-1-yl)-2-methyl-propanoate

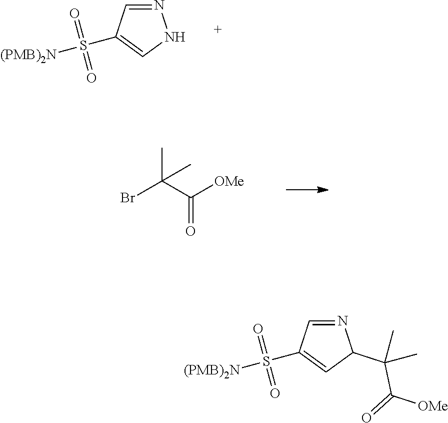

A suspension of N,N-bis(4-methoxybenzyl)-1H-pyrazole-4-sulfonamide (403.5 mg, 1.041 mmol), methyl 2-bromo-2-methylpropanoate (0.140 mL, 1.041 mmol) and K₂CO₃ (430 mg, 3.11 mmol) in MeCN (10 mL) was stirred at RT for 18 h, then at 50° C. for 4.5 h. The reaction mixture was partitioned between water (30 mL) and EtOAc (50 mL), the aqueous phase was separated and further extracted with EtAOc (3×50 mL). The organic phases were combined, dried (MgSO₄), filtered and concentrated in vacuo. The crude product was purified by FC (20-100% EtOAc/isohexane) to afford the title compound (424 mg, 82%) as a white solid.

LCMS m/z 488.4 (M+H)⁺ (ES⁺).

¹H NMR (DMSO-d₆) δ 8.40 (s, 1H), 7.86 (s, 1H), 7.11-7.06 (m, 4H), 6.85-6.80 (m, 4H), 4.17 (s, 4H), 3.72 (s, 6H), 3.65 (s, 3H), 1.77 (s, 6H).

### Step C: 1-(1-hydroxy-2-methylpropan-2-yl)-N,N-bis(4-methoxybenzyl)-1H-pyrazole-4-sulfonamide

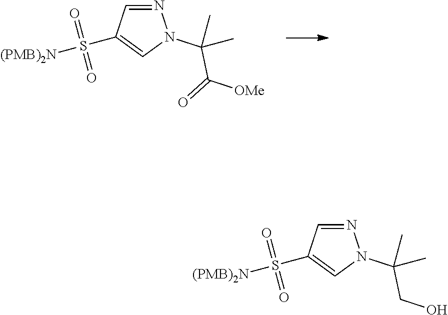

LiBH₄ (4 M in THF) (0.65 mL, 2.60 mmol) was added to a solution of methyl 2-(4-(N,N-bis(4-methoxybenzyl)sulfamoyl)-1H-pyrazol-1-yl)-2-methylpropanoate (423.8 mg, 0.869 mmol) in anhydrous THF (5 mL) at 0° C. The reaction was stirred for 1.5 h. The reaction mixture was partition between EtOAc (50 mL) and water (50 mL). The organic phase was separated and the aqueous was extracted with EtOAc (2×50 mL). The organic phases were combined, dried (MgSO₄), filtered and concentrated in vacuo to afford the title compound (392 mg, 93%) as a white sticky solid which was used without further purification.

LCMS m/z 460.5 (M+H)⁺ (ES⁺).

¹H NMR (DMSO-d₆) δ 8.14 (s, 1H), 7.80 (s, 1H), 7.09-7.04 (m, 4H), 6.84-6.80 (m, 4H), 5.08 (t, J=5.6 Hz, 1H), 4.15 (s, 4H), 3.71 (d, J=1.5 Hz, 6H), 3.58 (d, J=5.7 Hz, 2H), 1.45 (s, 6H).

### Intermediate A10: 2-(3-hydroxypropoxy)-N,N-bis(4-methoxybenzyl)pyridine-3-sulfonamide

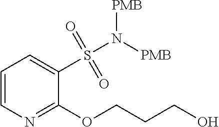

### Step A: 2-chloro-N,N-bis(4-methoxybenzyl)pyridine-3-sulfonamide

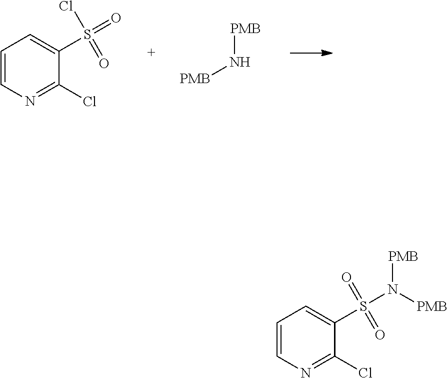

A suspension of 2-chloropyridine-3-sulfonyl chloride (1.5 g, 7.07 mmol) and bis(4-methoxybenzyl)amine (1.820 g, 7.07 mmol) in DCM (20 mL) was cooled to 0° C. (ice bath). TEA (2.51 mL, 17.68 mmol) was then added dropwise at 0° C. and the mixture was stirred at RT for 4 h. The mixture was diluted with water (30 mL) and the organic layer was separated. The aqueous layer was extracted with DCM (2×20 mL) and the combined organic layers were dried (MgSO₄), filtered and concentrated in vacuo to give a yellow oil. The yellow oil was purified by FC (0-50% EtOAc/isohexane) to afford the title compound (1.5 g, 49%) as a colourless solid.

LCMS m/z 433/435 (M+H)⁺ (ES⁺).

### Step B: 2-(3-hydroxypropoxy)-N,N-bis(4-methoxybenzyl)pyridine-3-sulfonamide

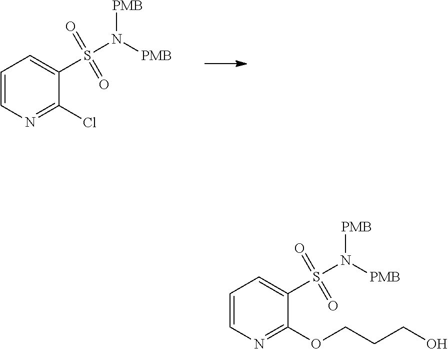

A mixture of propane-1,3-diol (2 mL, 3.46 mmol) and KO^{t}Bu (0.778 g, 6.93 mmol) in THF (10 mL) was stirred at RT for 10 min. 2-Chloro-N,N-bis(4-methoxybenzyl)-pyridine-3-sulfonamide (1.5 g, 3.46 mmol) was then added and the mixture was stirred at RT for a further 24 h. The mixture was then diluted with water (20 mL) and EtOAc (20 mL) and the organic layer was separated. The aqueous layer was extracted with EtOAc (2×20 mL) and the combined organic layers were dried (MgSO₄), filtered and concentrated in vacuo to give a brown oil. The crude product was purified by FC (0-50% EtOAc/isohexane) to afford the title compound (360 mg, 21%) as a colourless oil.

LCMS m/z 495.4 (M+Na)⁺ (ES⁺).

### Intermediate A11: 1-(1-hydroxy-2-methylpropan-2-yl)-4-methoxy-N,N-bis(4-methoxybenzyl)-1H-pyrazole-3-sulfonamide

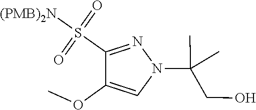

### Step A: 1-(tetrahydro-2H-pyran-2-yl)-1H-pyrazol-4-ol

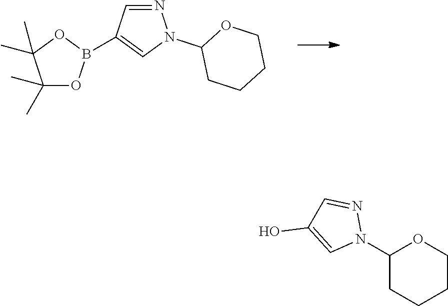 [SMILES: CC1(C)OB(c2cnn(C3CCCCO3)c2)OC1(C)C ; Oc1cnn(C2CCCCO2)c1]

1-(Tetrahydro-2H-pyran-2-yl)-4-(4,4,5,5-tetramethyl-1,3,2-dioxaborolan-2-yl)-1H-pyrazole (15.6 g, 56.1 mmol) was dissolved in THF (90 mL) and cooled to 0° C. NaOH (2 M) (56.1 mL, 112 mmol) was added and the mixture was stirred for 10 min before hydrogen peroxide (30% aq) (11.46 mL, 112 mmol) was added in 0.5 mL portions over the course of 10 min. The reaction was stirred for a further 3 h whilst allowing the reaction mixture to warm to RT. Sodium sulfite (sat aq) (150 mL) was added and stirred for 5 min. The pH of the reaction mixture was adjusted to pH 9, via addition of 2 M aq HCl, and then extracted with EtOAc (2×100 mL). The combined organics were washed with water (150 mL), brine (150 mL) and dried (MgSO₄). The organic layer was dry loaded onto silica and purified by FC (20-100% EtOAc/isohexane) to afford the title compound (2.77 g, 16.47 mmol) as a white solid. The combined aqueous layers were concentrated in vacuo and the resulting residue dissolved in MeOH, dry loaded onto silica and purified by FC (20-100% EtOAc/isohexane) affording further material which was combined with the first batch to afford the title compound (5.42 g, 58%).

LCMS m/z 84.9 (M+H-THP)⁺ (ES⁺).

¹H NMR (DMSO-d₆) δ 8.47 (s, 1H), 7.27 (s, 1H), 7.05 (s, 1H), 5.18 (dd, J=10.1, 2.5 Hz, 1H), 3.98-3.77 (m, 1H), 3.68-3.50 (m, 1H), 2.04-1.96 (m, 1H), 1.93-1.87 (m, 1H), 1.83-1.79 (m, 1H), 1.67-1.57 (m, 1H), 1.52-1.45 (m, 2H).

### Step B: 4-methoxy-1-(tetrahydro-2H-pyran-2-yl)-1H-pyrazole

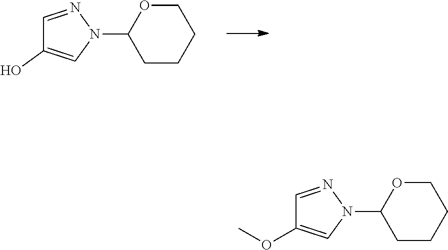

1-(Tetrahydro-2H-pyran-2-yl)-1H-pyrazol-4-ol (2.675 g, 15.90 mmol) was dissolved in dry DMF (10 mL), Cs₂CO₃ (6.22 g, 19.09 mmol) was added and the suspension was cooled to 0° C. under N₂. MeI (1.09 mL, 17.49 mmol) was added and the reaction mixture was allowed to reach RT and stirred for 16 h. The mixture was concentrated in vacuo and the residue was partitioned between water (10 mL) and EtOAc (20 mL). The aqueous was washed with more EtOAc (10×20 mL). The combined organics were dried (MgSO₄), filtered and concentrated in vacuo to give a yellow liquid. The crude product was purified by FC (0-50% EtOAc/isohexane) to afford the title compound (5.00 g, 56%) as a colourless liquid.

LCMS m/z 99.0 (M+H-THP)⁺ (ES⁺).

¹H NMR (CDCl₃) δ 7.30 (s, 1H), 7.26 (s, 1H), 5.28 (dt, J=9.5, 2.3 Hz, 1H), 4.06-4.00 (m, 1H), 3.74 (s, 3H), 3.68 (td, J=11.1, 3.0 Hz, 1H), 2.30-1.86 (m, 3H), 1.86-1.46 (m, 3H).

### Step C: lithium 4-methoxy-1-(tetrahydro-2H-pyran-2-yl)-1H-pyrazole-5-sulfinate

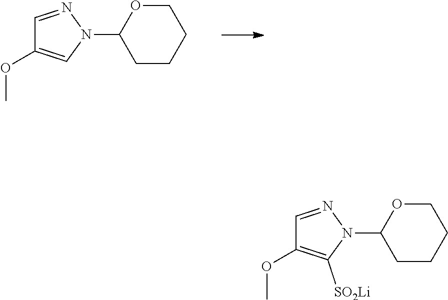

In a dry 500 mL 3 neck-flask equipped with thermometer and nitrogen inlet, 4-methoxy-1-(tetrahydro-2H-pyran-2-yl)-1H-pyrazole (5.00 g, 27.4 mmol) was dissolved in dry THF (200 mL) and cooled to −78° C. n-Butyllithium (2.5 M in hexanes) (12.07 ml, 30.2 mmol) was added in 0.5 mL portions over 10 min ensuring the internal reaction temperature did not exceed −65° C. The reaction mixture was stirred at −78° C. for 1 h. SO₂ gas was bubbled through the reaction mixture for 10 min and the mixture was allowed to reach RT overnight. The reaction mixture was filtered and the resulting solid washed with TBME, then isohexane, and then dried via desiccator to afford lithium 4-methoxy-1-(tetrahydro-2H-pyran-2-yl)-1H-pyrazole-5-sulfinate (5.52 g, 80%) as a white solid.

¹H NMR (DMSO-d₆): δ 7.12 (s, 1H), 6.19 (dd, J=10.1, 2.4 Hz, 1H), 3.98-3.84 (m, 1H), 3.67 (s, 3H), 3.52-3.47 (m, 1H), 2.20-2.02 (m, 1H), 1.98-1.86 (m, 1H), 1.73-1.64 (m, 1H), 1.62-1.51 (m, 1H), 1.51-1.43 (m, 2H).

### Step D: 4-methoxy-N,N-bis(4-methoxybenzyl)-1-(tetrahydro-2H-pyran-2-yl)-1H-pyrazole-5-sulfonamide

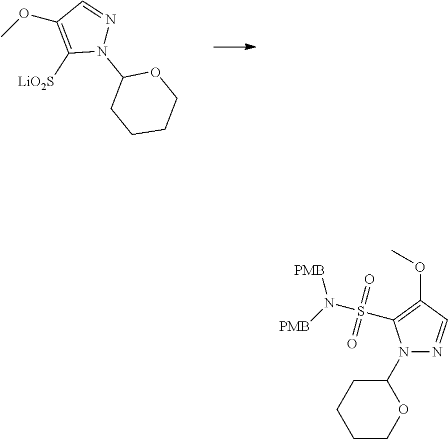

NCS (2.92 g, 21.88 mmol) was added to a suspension of lithium 4-methoxy-1-(tetrahydro-2H-pyran-2-yl)-1H-pyrazole-5-sulfinate (5.52 g, 21.88 mmol) in DCM (60 mL) cooled in an ice bath. The mixture was stirred for 1 h, quenched with water (50 mL) then partitioned between DCM (50 mL) and water (40 mL). The organic phase was washed with water (40 mL), dried using a phase separator, and concentrated in vacuo to −20 mL. This solution was then added to a mixture of bis(4-methoxybenzyl)amine (6.19 g, 24.07 mmol) and TEA (9.15 mL, 65.6 mmol) in DCM (50 mL) cooled in an ice bath. After stirring for 1 h the mixture was warmed to RT then partitioned between DCM (60 mL) and water (40 mL). The organic layer was washed with water (40 mL), aq 1 M HCl (2×40 mL), and water (40 mL), dried using a phase separator, and concentrated in vacuo to afford the title compound (9.27 g, 79%) as an orange oil.

LCMS m/z 524.2 (M+Na)⁺ (ES⁺).

¹H NMR (DMSO-d₆) δ 7.67 (s, 1H), 6.97 (d, J=8.5 Hz, 4H), 6.81 (d, J=8.4 Hz, 4H), 5.84 (dd, J=9.8, 2.4 Hz, 1H), 4.42-4.15 (m, 4H), 3.88-3.82 (m, 1H), 3.75 (s, 3H), 3.71 (s, 6H), 3.57-3.50 (m, 1H), 2.32-2.24 (m, 1H), 2.02-1.95 (m, 1H), 1.89-1.80 (m, 1H), 1.70-1.57 (m, 1H), 1.53-1.45 (m, 2H).

### Step E: 4-methoxy-N,N-bis(4-methoxybenzyl)-1H-pyrazole-5-sulfonamide

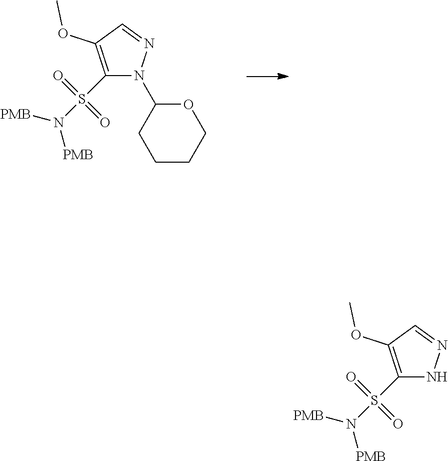

1 M HCl (aq) (20 mL) was added to a solution of 4-methoxy-N,N-bis(4-methoxy-benzyl)-1-(tetrahydro-2H-pyran-2-yl)-1H-pyrazole-5-sulfonamide (9.274 g, 18.49 mmol) in THF (50 mL) and MeOH (25 mL). The mixture was stirred at RT for 4 h then concentrated to −20 mL. The resulting residue was partitioned between EtOAc (100 mL) and water (40 mL), the organic layer washed with water (40 mL), dried using a phase separator, and concentrated in vacuo. The resulting solid precipitate was filtered, washed with TBME and isohexane, and dried to afford the title compound (6.106 g, 79%) as a white solid.

LCMS m/z 418.2 (M+H)⁺ (ES⁺).

¹H NMR (DMSO-d₆) δ 13.26 (s, 1H), 7.76 (s, 1H), 7.00 (d, J=8.7 Hz, 4H), 6.79 (d, J=8.6 Hz, 4H), 4.21 (s, 4H), 3.73 (s, 3H), 3.71 (s, 6H).

### Step F: 1-(1-hydroxy-2-methylpropan-2-yl)-4-methoxy-N,N-bis(4-methoxybenzyl)-1H-pyrazole-3-sulfonamide

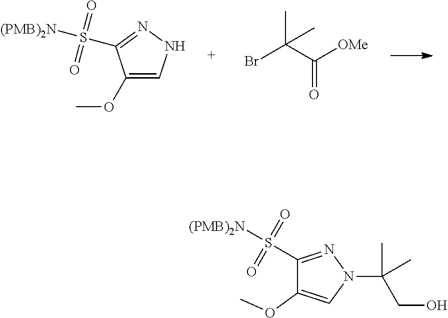

A suspension of 4-methoxy-N,N-bis(4-methoxybenzyl)-1H-pyrazole-5-sulfonamide (2 g, 4.79 mmol), methyl 2-bromo-2-methylpropanoate (0.70 mL, 5.41 mmol) and K₂CO₃ (1.980 g, 14.32 mmol) in MeCN (30 mL) was stirred at 50° C. for 5 h. The reaction mixture was partitioned between water (70 mL) and EtOAc (100 mL), and the aqueous phase was separated and further extracted with EtOAc (2×100 mL). The organic phases were combined, dried (MgSO₄), filtered and concentrated in vacuo to afford methyl 2-(3-(N,N-bis(4-methoxybenzyl)sulfamoyl)-4-methoxy-1H-pyrazol-1-yl)-2-methylpropanoate as a colourless oil. The crude was taken up in dry THF (30 mL) and the solution cooled to 0° C., then LiBH₄ (4 M in THF) (7.50 mL, 15.00 mmol) was added and the resulting solution was stirred at RT for 1.5 h. The reaction was quenched by the careful addition of MeOH, then the mixture was partitioned between EtOAc (100 mL) and water (100 mL). The aqueous phase was separated and further extracted with EtOAc (2×100 mL). The organic phases were combined, dried (MgSO₄), filtered and concentrated in vacuo. The crude product was purified by FC (50-100% EtOAc/isohexane) to afford the title compound (1.81 g, 3.51 mmol) as a colourless oil.

LCMS m/z 512.5 (M+Na)⁺ (ES⁺).

¹H NMR (DMSO-d₆) δ 7.80 (s, 1H), 7.07-6.92 (m, 4H), 6.88-6.67 (m, 4H), 4.20 (s, 4H), 3.73-3.69 (m, 9H), 3.57 (d, J=5.5 Hz, 2H), 1.45 (s, 6H). One exchangeable proton not observed.

### Intermediate A12: 4-fluoro-1-(1-(hydroxymethyl)cyclopropyl)-N,N-bis(4-methoxy-benzyl)-1H-pyrazole-3-sulfonamide

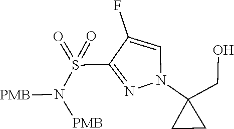

### Step A: methyl 1-(3-(N,N-bis(4-methoxybenzyl)sulfamoyl)-4-fluoro-1H-pyrazol-1-yl)-cyclopropanecarboxylate

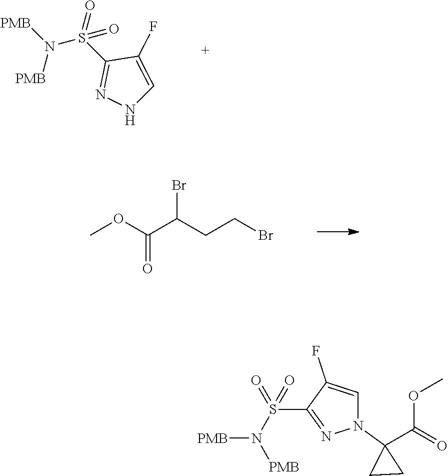

60 wt % NaH in mineral oil (0.50 g, 12.50 mmol) was added portion wise to a solution of 4-fluoro-N,N-bis(4-methoxybenzyl)-1H-pyrazole-3-sulfonamide (Intermediate A4, Step E) (2.00 g, 4.93 mmol) in NMP (30 mL) cooled to 0° C. The mixture was stirred at 0° C. for 10 min until gas evolution ceased. Methyl 2,4-dibromobutanoate (1.05 mL, 7.43 mmol) was then added and the mixture was left to warm to RT with stirring over 18 h. The mixture was then quenched with water (10 mL) and partitioned between brine (300 mL) and MTBE (100 mL). The aqueous layer was discarded and the organic layer was washed with brine (300 mL). The organic layer was dried (MgSO₄), filtered and concentrated in vacuo to give a brown oil. The crude product was purified by FC (0-60% EtOAc/isohexane) to afford the title compound (1.72 g, 60%) as a thick colourless oil.

LCMS m/z 526.4 (M+Na)⁺ (ES⁺).

¹H NMR (DMSO-d₆) δ 8.35 (d, J=4.6 Hz, 1H), 7.08-7.00 (m, 4H), 6.88-6.76 (m, 4H), 4.25 (s, 4H), 3.73 (s, 6H), 3.65 (s, 3H), 1.80-1.72 (m, 2H), 1.66-1.58 (m, 2H).

### Step B: 4-fluoro-1-(1-(hydroxymethyl)cyclopropyl)-N,N-bis(4-methoxybenzyl)-1H-pyrazole-3-sulfonamide

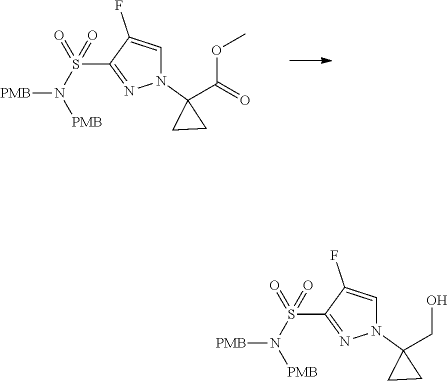

4 M LiBH₄ in THF (1.30 mL, 5.20 mmol) was added dropwise to a stirred solution of methyl 1-(3-(N,N-bis(4-methoxybenzyl)sulfamoyl)-4-fluoro-1H-pyrazol-1-yl)-cyclopropanecarboxylate (1.72 g, 2.94 mmol) in THF (30 mL) cooled to 0° C. The mixture was stirred for 17 h. The mixture was then partitioned between water (20 mL) and EtOAc (50 mL). The organic layer was collected and the aqueous layer was extracted with EtOAc (2×20 mL). The combined organic layers were dried (MgSO₄), filtered and concentrated in vacuo to give the title compound (1.36 g, 89%) as a thick pale yellow oil which was used without further purification.

LCMS m/z 498.4 (M+Na)⁺ (ES⁺).

¹H NMR (DMSO-d₆) δ 8.15 (d, J=4.7 Hz, 1H), 7.06-6.98 (m, 4H), 6.86-6.78 (m, 4H), 5.12 (t, J=5.8 Hz, 1H), 4.24 (s, 4H), 3.72 (s, 6H), 3.62 (d, J=6.0 Hz, 2H), 1.14-1.07 (m, 2H), 1.07-0.96 (m, 2H).

### Intermediate A13: 1-(1-hydroxy-2-methylpropan-2-yl)-N,N-bis(4-methoxybenzyl)-3,5-dimethyl-1H-pyrazole-4-sulfonamide

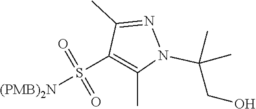

### Step A: N,N-bis(4-methoxybenzyl)-3,5-dimethyl-1H-pyrazole-4-sulfonamide

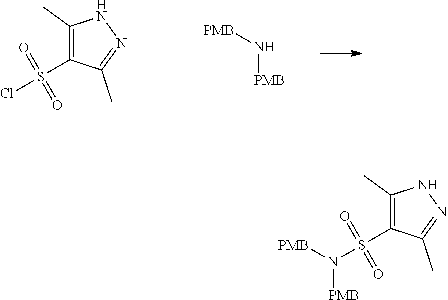

A solution of bis(4-methoxybenzyl)amine (1.49 g, 5.78 mmol) in DCM (5 mL, 3.85 mmol) was prepared. The reaction mixture was then cooled to 0° C. followed by the dropwise addition of 3,5-dimethyl-1H-pyrazole-4-sulfonyl chloride (0.75 g, 3.85 mmol) and TEA (1.61 mL, 11.56 mmol) in DCM (5 mL). The resulting reaction mixture was stirred at RT for 2 h. The reaction mixture was diluted with water (10 mL) and DCM (10 mL) and the organic layer dried (phase separator) and concentrated in vacuo to yield a yellow oil. The crude product was purified by FC (0-50% EtOAc/isohexane) to afford the title compound (760 mg, 40%) as a colourless oil.

LCMS m/z 416.8 (M+H)⁺ (ES⁺).

¹H NMR (DMSO-d₆) δ 13.01 (s, 1H), 7.00-6.93 (m, 4H), 6.86-6.80 (m, 4H), 4.15 (s, 4H), 3.72 (s, 6H), 2.31 (s, 6H).

### Step B: methyl 2-(4-(N,N-bis(4-methoxybenzyl)sulfamoyl)-3,5-dimethyl-1H-pyrazol-1-yl)-2-methylpropanoate

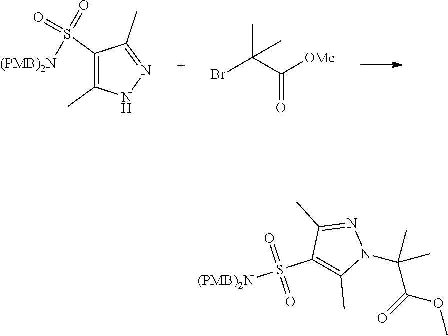

A suspension of N,N-bis(4-methoxybenzyl)-3,5-dimethyl-1H-pyrazole-4-sulfonamide (670 mg, 1.61 mmol), methyl 2-bromo-2-methylpropanoate (0.271 ml, 2.10 mmol) and potassium carbonate (669 mg, 4.84 mmol) in DMF (20 mL) was stirred at 80° C. for 16 h. The reaction mixture was partitioned between water (30 mL) and EtOAc (50 mL), the aqueous phase was separated and further extracted with EtOAc (3×50 mL). The organic phases were combined, washed with brine (30 mL), extracted, dried (phase separator), and concentrated in vacuo. The crude product was purified by FC (0-50% EtOAc/isohexane) to afford the title compound (318 mg, 36%) as a pale yellow oil.

LCMS m/z 516.1 (M+H)⁺ (ES⁺).

¹H NMR (DMSO-d₆) δ 6.99-6.94 (m, 4H), 6.87-6.82 (m, 4H), 4.17 (s, 4H), 3.73 (s, 9H), 2.27 (d, J=3.7 Hz, 6H), 1.74 (s, 6H).

### Step C: 1-(1-hydroxy-2-methylpropan-2-yl)-N,N-bis(4-methoxybenzyl)-3,5-dimethyl-1H-pyrazole-4-sulfonamide

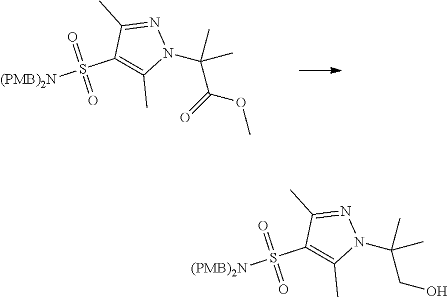

4 M LiBH₄ in THF (0.5 mL, 2.00 mmol) was added dropwise to a stirred solution of methyl 2-(4-(N,N-bis(4-methoxybenzyl)sulfamoyl)-3,5-dimethyl-1H-pyrazol-1-yl)-2-methylpropanoate (318 mg, 0.617 mmol) in THF (12 mL) cooled to 0° C. The mixture was stirred for 16 h. Additional 4 M LiBH₄ in THF (0.5 mL, 2.000 mmol) was added at 0° C. and allowed to stir for a further 60 h. The mixture was then partitioned between water (20 mL) and EtOAc (50 mL). The organic layer was collected and the aqueous layer was extracted with EtOAc (2×20 mL). The combined organic layers were dried (phase separator) and concentrated in vacuo. The crude product was purified by FC (0-100% EtOAc/isohexane) to afford the title compound (206 mg, 62%) as a sticky colourless oil.

LCMS m/z 488.0 (M+H)⁺ (ES⁺).

¹H NMR (DMSO-d₆) δ 6.99-6.92 (m, 4H), 6.87-6.79 (m, 4H), 5.08-5.02 (m, 1H), 4.18-4.12 (m, 4H), 3.75-3.70 (m, 6H), 3.68-3.63 (m, 2H), 3.35-3.27 (m, 3H), 2.27-2.22 (m, 3H), 1.56-1.49 (m, 6H).

### Intermediate A14: 1-(2-hydroxyethyl)-N,N-bis(4-methoxybenzyl)-1H-pyrazole-4-sulfonamide

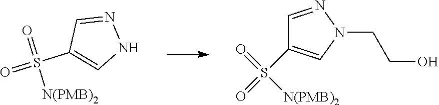

N,N-bis(4-methoxybenzyl)-1H-pyrazole-4-sulfonamide (Intermediate A9, Step A) (1.50 g, 3.68 mmol) and K₂CO₃ (1.27 g, 9.19 mmol) were suspended in MeCN (25 mL) under a N₂ atmosphere. 2-Bromoethanol (0.30 mL, 4.05 mmol) was added and the mixture was heated to 50° C. for 4 h. After cooling to RT, water (50 mL) and EtOAc (75 mL) were added and the organics separated. The organic phase was dried (MgSO₄), filtered and concentrated in vacuo. The crude product was purified by FC (0-100% EtOAc/isohexane) to afford the title compound (1.29 g, 80%) as a white solid.

LCMS m/z 432.4 (M+H)⁺ (ES⁺).

¹H NMR (DMSO-d₆) δ 8.30 (s, 1H), 7.81 (s, 1H), 7.08-7.03 (m, 4H), 6.84-6.78 (m, 4H), 4.98 (t, J=5.3 Hz, 1H), 4.19 (t, J=5.5 Hz, 2H), 4.13 (s, 4H), 3.76 (q, J=5.4 Hz, 2H), 3.71 (s, 6H).

### Intermediate A15: 1-(1-hydroxy-2-methylpropan-2-yl)-N,N-bis(4-methoxybenzyl)-3-methyl-1H-pyrazole-4-sulfonamide

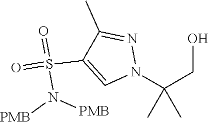

### Step A: methyl 2-methyl-2-(3-methyl-1H-pyrazol-1-yl)propanoate

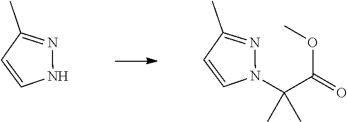

A solution of 5-methyl-1H-pyrazole (1.00 mL, 12.94 mmol) and methyl 2-bromo-2-methylpropanoate (2.2 mL, 17.00 mmol) in THF (20 mL) was cooled to 0° C. and NaH (60 wt %) (673 mg, 16.83 mmol) was added and stirred for 10 min. Then methyl 2-bromo-2-methylpropanoate (2.2 mL, 17.00 mmol) was added and stirred at RT over the weekend. The reaction mixture was partitioned between water (30 mL) and EtOAc (50 mL), and the aqueous phase was separated and further extracted with EtAOc (3×50 mL). The organic phases were combined, washed with brine (30 mL), extracted, dried (phase separator), and concentrated under reduced pressure. The crude product was purified by FC (0-100% EtOAc/isohexane) to afford the title compound (1.0 g, 40%) as a colourless oil.

¹H NMR (DMSO-d₆) δ 7.74 (d, J=2.3 Hz, 1H), 6.04 (d, J=2.3 Hz, 1H), 3.61 (s, 3H), 2.14 (s, 3H), 1.71 (s, 6H).

### Step B: methyl 2-(4-(N,N-bis(4-methoxybenzyl)sulfamoyl)-3-methyl-1H-pyrazol-1-yl)-2-methylpropanoate

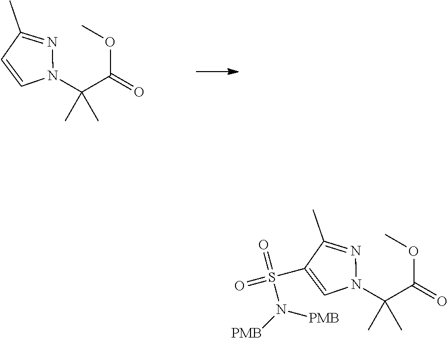

To a solution of chlorosulfonic acid (2.0 mL, 30 mmol) in CHCl₃ (5 mL) at 0° C. was added a solution of methyl 2-methyl-2-(3-methyl-1H-pyrazol-1-yl)propanoate (1.0 g, 5.5 mmol) in CHCl₃ (5 mL) over 2 min. The mixture was refluxed at 60° C. and stirred for 24 h. The reaction was cooled to RT and thionyl chloride (0.44 mL, 6.0 mmol) was added and the reaction was heated at 60° C. for a further 2 h. The reaction mixture was cooled to RT and added to a stirred mixture of DCM (50 mL) and ice water (50 mL). The organic layer was separated and the aqueous layer was extracted with DCM (2×20 mL). The combined organics were dried (MgSO₄), filtered and concentrated in vacuo to afford an orange oil (1.00 g). The orange oil was dissolved in DCM (10 mL) and bis(4-methoxybenzyl)amine (1.4 g, 55 mmol) followed by the slow addition of TEA (1.1 mL, 8.2 mmol) at 0° C. The reaction mixture was allowed to warm to RT and stirred overnight. The mixture was diluted with water (30 mL) and the organic layer was separated. The aqueous layer was extracted with DCM (2×20 mL) and the combined organic layers were dried (MgSO₄), filtered and concentrated in vacuo. The crude yellow oil was purified by FC (0-100% EtOAc/isohexane) to afford the title compound (1.17 g, 36%) as a yellow oil, which solidified on standing to form a yellow solid.

LCMS m/z 502.2 (M+H)⁺ (ES⁺).

¹H NMR (DMSO-d₆) δ 8.29 (s, 1H), 7.06-6.97 (m, 4H), 6.88-6.74 (m, 4H), 4.20 (s, 4H), 3.72 (s, 6H), 3.64 (s, 3H), 2.24 (s, 3H), 1.75 (s, 6H).

### Step C: 1-(1-hydroxy-2-methylpropan-2-yl)-N,N-bis(4-methoxybenzyl)-3-methyl-1H-pyrazole-4-sulfonamide

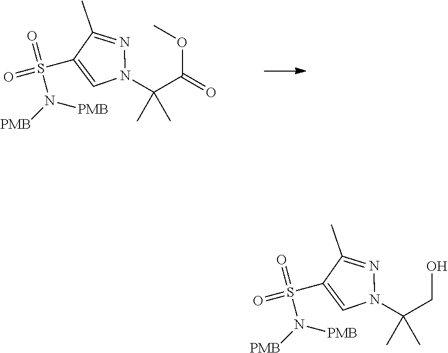

LiBH₄ (4 M in THF) (2 mL, 9.33 mmol) was added dropwise to a stirred solution of methyl 2-(4-(N,N-bis(4-methoxybenzyl)sulfamoyl)-3-methyl-1H-pyrazol-1-yl)-2-methylpropanoate (1.17 g, 2.33 mmol) in THF (15 mL) cooled to 0° C. The mixture was stirred for 18 h. The mixture was then partitioned between water (100 mL) and EtOAc (100 mL). The organic layer was collected and the aqueous layer was extracted with EtOAc (2×50 mL). The combined organic layers were dried (MgSO₄), filtered and concentrated in vacuo to afford the title compound (1.04 g, 82%) as a pale yellow oil.

LCMS m/z 474.5 (M+H)⁺ (ES⁺).

¹H NMR (DMSO-d₆) δ 8.03 (s, 1H), 7.05-6.98 (m, 4H), 6.84-6.79 (m, 4H), 5.05 (t, J=5.5 Hz, 1H), 4.18 (s, 4H), 3.71 (s, 6H), 3.56 (d, J=5.7 Hz, 2H), 2.26 (s, 3H), 1.43 (s, 6H).

### Intermediate A16: 2-fluoro-5-(2-hydroxyethyl)-N,N-bis(4-methoxybenzyl)benzene-sulfonamide

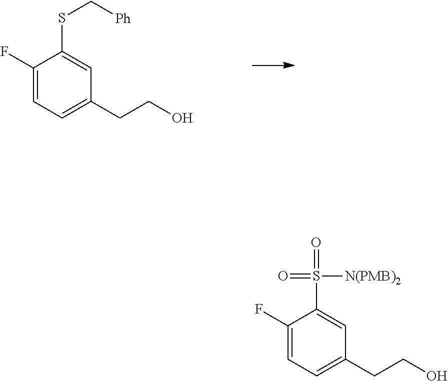

A solution of 2-(3-(benzylthio)-4-fluorophenyl)ethanol (4.17 g, 15.90 mmol) in MeCN (90 mL), AcOH (1.05 mL) and water (2.1 mL) was cooled to −10° C. (ice/acetone bath). 1,3-Dichloro-5,5-dimethylimidazolidine-2,4-dione (4.70 g, 23.84 mmol) was then added and the mixture was stirred at −10° C. for 4 h. The mixture was then partitioned between DCM (50 mL) and water (50 mL) and the organic layer was collected. The aqueous layer was extracted with DCM (100 mL) and the combined organic layers were dried (MgSO₄), filtered and concentrated in vacuo. The resulting residue was taken up in DCM (100 mL) and cooled to 0° C. bis-(4-Methoxybenzyl)amine (4.17 g, 16.21 mmol) and TEA (4.43 mL, 31.8 mmol) were then added to the solution, which was then allowed to warm to RT and stirred for 18 h. The reaction mixture was quenched with water (75 mL) then partitioned between DCM (100 mL) and water (25 mL). The organic phase was separated, and the aqueous layer re-extracted with DCM (2×50 mL). The organics were combined, dried (MgSO₄) and concentrated in vacuo. The crude product was purified by FC (0-80% EtOAc/isohexane) to afford the title compound (2.68 g, 32%) as a pale yellow oil.

LCMS m/z 482.5 (M+Na)⁺ (ES⁺).

¹H NMR (DMSO-d₆) δ 7.62 (dd, J=7.0, 2.3 Hz, 1H), 7.56-7.52 (m, 1H), 7.36-7.29 (m, 1H), 7.02-6.96 (m, 4H), 6.81-6.77 (m, 4H), 4.68 (t, J=5.1 Hz, 1H), 4.27 (s, 4H), 3.71 (s, 6H), 3.64-3.58 (m, 2H), 2.75 (t, J=6.5 Hz, 2H).

### Intermediate A17: 3-(1-hydroxy-2-methylpropan-2-yl)-N,N-bis(4-methoxybenzyl)-benzenesulfonamide

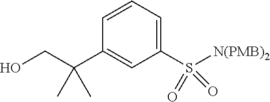

### Step A: methyl 2-(3-(benzylthio)phenyl)acetate

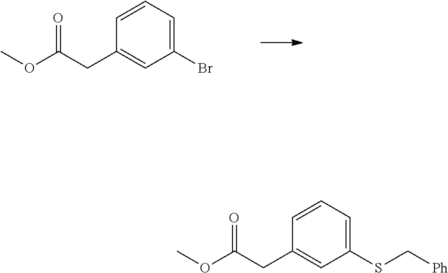 [SMILES: COC(=O)Cc1cccc(Br)c1 ; COC(=O)Cc1cccc(SCc2ccccc2)c1]

Benzyl mercaptan (1.54 mL, 13.10 mmol) was added to a stirred, N₂-degassed solution of methyl 2-(3-bromophenyl)acetate (3 g, 13.10 mmol), XantPhos (0.76 g, 1.310 mmol), DIPEA (4.57 mL, 26.2 mmol) and Pd₂(dba)₃ (0.60 g, 0.655 mmol) in 1,4-dioxane (80 mL) at RT. The mixture was then heated to 100° C. for 17 h. The mixture was filtered through Celite® and dry-loaded onto silica gel (30 g). The crude product was purified by FC (0-40% DCM/isohexane) to afford the title compound (3.22 g, 89%) as an orange oil.

¹H NMR (DMSO-d₆) δ 7.37-7.33 (m, 2H), 7.31-7.27 (m, 2H), 7.26-7.20 (m, 4H), 7.09-7.04 (m, 1H), 4.23 (s, 2H), 3.64 (s, 2H), 3.60 (s, 3H).

### Step B: methyl 2-(3-(N,N-bis(4-methoxybenzyl)sulfamoyl)phenyl)acetate

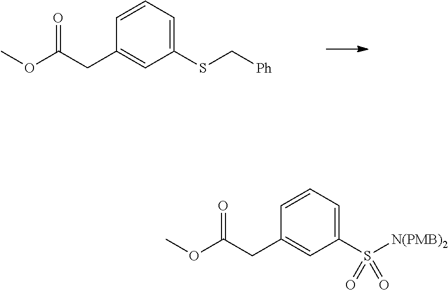

A solution of methyl 2-(3-(benzylthio)phenyl)acetate (1.62 g, 5.95 mmol) in MeCN (29 mL), AcOH (0.4 mL) and water (0.8 mL) was cooled to −10° C. (ice/acetone bath). 1,3-dichloro-5,5-dimethylimidazolidine-2,4-dione (1.758 g, 8.92 mmol) was then added and the mixture was stirred at −10° C. for 4 h. The mixture was then partitioned between DCM (50 mL) and water (50 mL) and the organic layer was collected. The aqueous layer was extracted with DCM (100 mL) and the combined organic layers were dried (MgSO₄), filtered and concentrated to dryness to give crude methyl 2-(3-(chloro-sulfonyl)phenyl)acetate as a thick yellow paste. bis-(4-Methoxybenzyl)amine (1.53 g, 5.95 mmol) was added to a suspension of methyl 2-(3-(chlorosulfonyl)phenyl)acetate (1.48 g, 5.95 mmol) in DCM (25 mL) cooled in an ice bath followed by TEA (1.5 mL, 10.76 mmol). The mixture was stirred for 17 h, quenched with water (20 mL) then partitioned between DCM (50 mL) and water (40 mL). The organic phase was collected, dried (MgSO₄), filtered and concentrated to dryness to give a brown oil. The brown oil was purified by chromatography on silica gel (80 g cartridge, 0-50% EtOAc/isohexane) to afford the title compound (2.45 g, 79%) as a white solid. Yield over 2 steps.

LCMS m/z 492.0 (M+Na)⁺ (ES⁺).

¹H NMR (DMSO-d₆) δ 7.79-7.69 (m, 2H), 7.62-7.50 (m, 2H), 7.02-6.94 (m, 4H), 6.85-6.73 (m, 4H), 4.19 (s, 4H), 3.84 (s, 2H), 3.71 (s, 6H), 3.64 (s, 3H).

### Step C: methyl 2-(3-(N,N-bis(4-methoxybenzyl)sulfamoyl)phenyl)-2-methylpropanoate

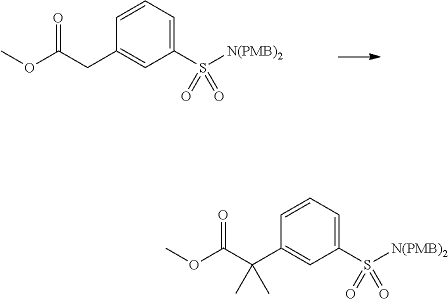

Methyl 2-(3-(N,N-bis(4-methoxybenzyl)sulfamoyl)phenyl)acetate (2.00 g, 4.26 mmol) was dissolved in THF (2 mL) at RT and 1 M lithium bis(trimethylsilyl)amide in THF (9 mL, 9.37 mmol) was added. The mixture was stirred at RT for 5 min and iodomethane (583 μL, 9.37 mmol) was added. The mixture was stirred at RT for 1 h. Additional 1 M lithium bis(trimethylsilyl)amide in THF (9 mL, 9.37 mmol) and iodomethane (583 μL, 9.37 mmol) were added and the mixture was stirred for a further 1 h, but no change was recorded. The mixture was then quenched with 1 M HCl (30 mL) and diluted with DCM (100 mL). The organic layer was collected and aqueous layer was extracted with DCM (50 mL). The combined organic layers were dried (MgSO₄), filtered and concentrated to dryness to give a brown oil. The crude product was purified twice by FC (0-50% EtOAc/isohexane, and then again with 0-50% EtOAc/isohexane) to afford the title compound (0.54 g, 23%) as a yellow oil.

LCMS m/z 520.0 (M+Na)⁺ (ES⁺).

¹H NMR (DMSO-d₆) δ 7.76 (dt, J=7.7, 1.4 Hz, 1H), 7.68-7.61 (m, 2H), 7.58 (t, J=7.7 Hz, 1H), 6.99-6.92 (m, 4H), 6.81-6.77 (m, 4H), 4.20 (s, 4H), 3.71 (s, 6H), 3.60 (s, 3H), 1.53 (s, 6H).

### Step D: 3-(1-hydroxy-2-methylpropan-2-yl)-N,N-bis(4-methoxybenzyl)benzene-sulfonamide

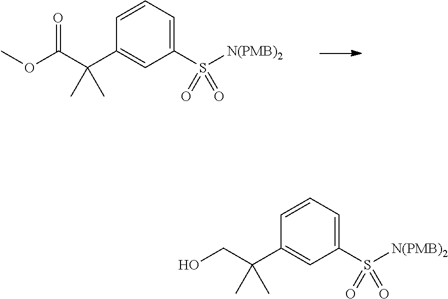

4 M LiBH₄ in THF (0.4 mL, 1.7 mmol) was added slowly to a stirred solution of methyl 2-(3-(N,N-bis(4-methoxybenzyl)sulfamoyl)phenyl)-2-methylpropanoate (0.54 g, 0.87 mmol) in THF (9 mL) cooled to 0° C. The mixture was then stirred for 17 h at RT. Additional 4 M LiBH₄ in THF (0.4 mL, 1.7 mmol) was then added and the mixture was stirred for a further 3 h at RT. The mixture was then cooled to 0° C. and carefully quenched with water (20 mL) followed by 1 M aq HCl (20 mL). The mixture was extracted with EtOAc (3×30 mL) and the combined organic layers were dried (MgSO₄), filtered and concentrated to dryness to give a yellow oil. The crude product was purified by FC (0-100% EtOAc/isohexane) to afford the title compound (0.47 g, 92%) as a thick colourless oil.

LCMS m/z 492.5 (M+Na)⁺ (ES⁺).

¹H NMR (DMSO-d₆) δ 7.74 (t, J=1.9 Hz, 1H), 7.72-7.64 (m, 2H), 7.55-7.49 (m, 1H), 6.99-6.92 (m, 4H), 6.80-6.75 (m, 4H), 4.80 (t, J=5.2 Hz, 1H), 4.18 (s, 4H), 3.70 (s, 6H), 3.45 (d, J=5.2 Hz, 2H), 1.23 (s, 6H).

### Intermediate A18: 3-cyclopropyl-5-(2-hydroxyethyl)-N,N-bis(4-methoxybenzyl)-benzenesulfonamide

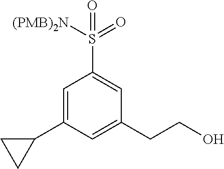

### Step A: methyl 2-(3-(benzylthio)-5-bromophenyl)acetate

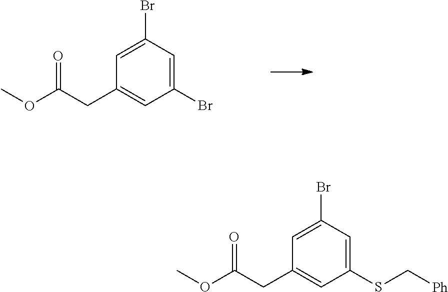 [SMILES: COC(=O)Cc1cc(Br)cc(Br)c1 ; COC(=O)Cc1cc(Br)cc(SCc2ccccc2)c1]

Prepared according to the general procedure of methyl 2-(3-(benzylthio)phenyl)acetate (Intermediate A17, Step A) from methyl 2-(3,5-dibromophenyl)acetate and benzyl mercaptan to afford the title compound (7.00 g, 59%) as a yellow oil.

¹H NMR (DMSO-d₆) δ 7.41-7.34 (m, 3H), 7.34-7.27 (m, 3H), 7.27-7.21 (m, 2H), 4.29 (s, 2H), 3.68 (s, 2H), 3.61 (s, 3H).

### Step B: methyl 2-(3-(N,N-bis(4-methoxybenzyl)sulfamoyl)-5-bromophenyl)acetate

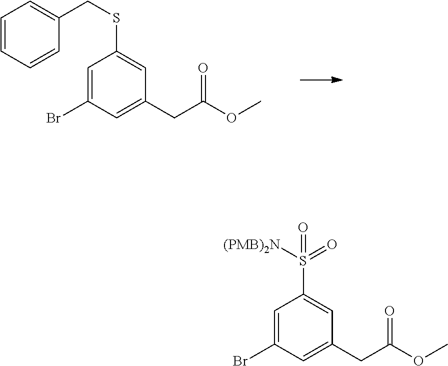

Prepared according to the general procedure of methyl 2-(3-(N,N-bis(4-methoxy-benzyl)sulfamoyl)phenyl)acetate (Intermediate A17, Step B) from methyl 2-(3-(benzylthio)-5-bromophenyl)acetate to afford the title compound (6.64 g, 55%) as an off-white solid.

LCMS m/z 546.2/548.3 (M−H)⁻ (ES⁻).

¹H NMR (DMSO-d₆) δ 7.80 (t, J=1.7 Hz, 1H), 7.75 (t, J=1.7 Hz, 1H), 7.68 (t, J=1.8 Hz, 1H), 7.07-7.02 (m, 4H), 6.84-6.79 (m, 4H), 4.25 (s, 4H), 3.85 (s, 2H), 3.73 (s, 6H), 3.65 (s, 3H).

### Step C: methyl 2-(3-(N,N-bis(4-methoxybenzyl)sulfamoyl)-5-cyclopropylphenyl)-acetate

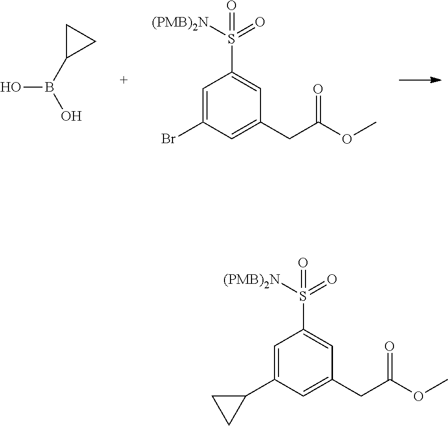

Prepared according to the general procedure of methyl 2-(2-cyclopropyl-6-(2-fluoro-pyridin-4-yl)phenyl)acetate (Intermediate B6, Step B) from methyl 2-(3-(N,N-bis-(4-methoxybenzyl)sulfamoyl)-5-bromophenyl)acetate and cyclopropyl boronic acid to afford the title compound (881 mg, 43%) as a light brown solid.

LCMS m/z 508.5 (M−H)⁻ (ES⁻).

¹H NMR (DMSO-d₆) δ 7.51 (m, 1H), 7.32 (m, 1H), 7.26 (m, 1H), 7.03-6.98 (m, 4H), 6.83-6.77 (m, 4H), 4.18 (s, 4H), 3.78 (s, 2H), 3.72 (s, 6H), 3.63 (s, 3H), 2.05-1.98 (m, 1H), 1.03-0.97 (m, 2H), 0.72-0.66 (m, 2H).

### Step D: 3-cyclopropyl-5-(2-hydroxyethyl)-N,N-bis(4-methoxybenzyl)-benzene-sulfonamide

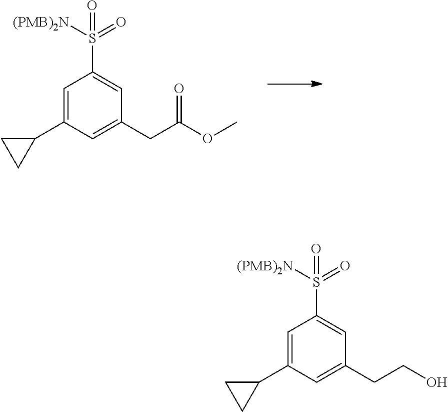

Methyl 2-(3-(N,N-bis(4-methoxybenzyl)sulfamoyl)-5-cyclopropylphenyl)acetate (881 mg, 1.729 mmol) was dissolved in dry THF (30 mL), placed under N₂ and cooled to 0° C. LiAlH₄ (2 M in THF) (978 μL, 1.956 mmol) was added drop-wise, and the reaction mixture warmed to RT, then allowed to stir for 16 h. The reaction was quenched with slow addition of water (20 mL), then diluted with brine (50 mL) and extracted with EtOAc (2×50 mL). The combined organic extracts were dried using a phase separator and concentrated in vacuo to afford the title compound (824 mg, 98%) as a yellow solid.

¹H NMR (DMSO-d₆) δ 7.42 (t, J=1.7 Hz, 1H), 7.26 (t, J=1.8 Hz, 1H), 7.21 (t, J=1.7 Hz, 1H), 7.03-6.98 (m, 4H), 6.83-6.77 (m, 4H), 4.68 (t, J=5.1 Hz, 1H), 4.18 (s, 4H), 3.72 (s, 6H), 3.65-3.59 (m, 2H), 2.76 (t, J=6.7 Hz, 2H), 2.03-1.96 (m, 1H), 1.02-0.96 (m, 2H), 0.72-0.67 (m, 2H).

### Intermediate A19: 4-fluoro-1-(4-hydroxy-2-methylbutan-2-yl)-N,N-bis(4-methoxy-benzyl)-1H-pyrazole-3-sulfonamide

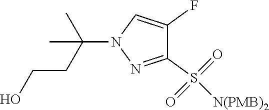

### Step A: ethyl 3-(3-(N,N-bis(4-methoxybenzyl)sulfamoyl)-4-fluoro-1H-pyrazol-1-yl)-3-methylbutanoate

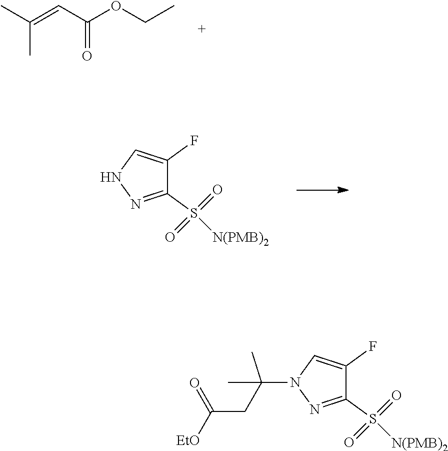

A solution of 4-fluoro-N,N-bis(4-methoxybenzyl)-1H-pyrazole-3-sulfonamide (Intermediate A4, Step E) (1.5 g, 3.7 mmol) and ethyl 3-methylbut-2-enoate (0.77 mL, 5.5 mmol) in MeCN (20 mL) was treated with DBU (0.56 mL, 3.7 mmol) and allowed to stir at 50° C. for 18 h. The reaction mixture was cooled to RT and concentrated in vacuo to afford a colourless oil. The crude product was purified by FC (0-50% EtOAc/isohexane) to afford the title compound (528 mg, 26%) as a colourless oil.

LCMS m/z 555.9 (M+Na)⁺ (ES⁺).

¹H NMR (DMSO-d₆) δ 8.30 (d, J=4.5 Hz, 1H), 7.10-7.00 (m, 4H), 6.88-6.78 (m, 4H), 4.23 (s, 4H), 3.96 (q, J=7.1 Hz, 2H), 3.73 (s, 6H), 2.90 (s, 2H), 1.60 (s, 6H), 1.09 (t, J=7.1 Hz, 3H).

### Step B: 4-fluoro-1-(4-hydroxy-2-methylbutan-2-yl)-N,N-bis(4-methoxybenzyl)-1H-pyrazole-3-sulfonamide

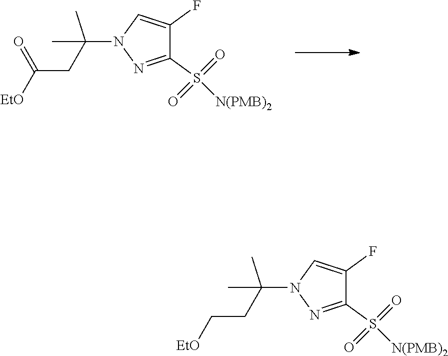

Prepared according to the general procedure of 3-(1-hydroxy-2-methylpropan-2-yl)-N,N-bis(4-methoxybenzyl)benzenesulfonamide (Intermediate A17, Step D) from ethyl 3-(3-(N,N-bis(4-methoxybenzyl)sulfamoyl)-4-fluoro-1H-pyrazol-1-yl)-3-methylbutanoate and lithium borohydride to afford the title compound (267 mg, 54%) as a colourless oil.

LCMS m/z 514.4 (M+Na)⁺ (ES⁺).

¹H NMR (DMSO-d₆) δ 8.25 (d, J=4.5 Hz, 1H), 7.05 (d, J=8.6 Hz, 4H), 6.83 (d, J=8.6 Hz, 4H), 4.47 (t, J=5.0 Hz, 1H), 4.24 (s, 4H), 3.72 (s, 6H), 3.30-3.23 (m, 2H), 1.98 (t, J=7.1 Hz, 2H), 1.51 (s, 6H).

### Intermediate A20: 1-(1-(hydroxymethyl)cyclopropyl)-N,N-bis(4-methoxybenzyl)-1H-pyrazole-4-sulfonamide

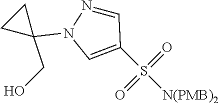

### Step A: methyl 1-(4-(N,N-bis(4-methoxybenzyl)sulfamoyl)-1H-pyrazol-1-yl)-cyclopropane-1-carboxylate

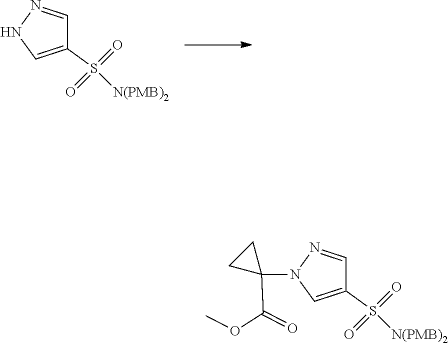

To a solution of N,N-bis(4-methoxybenzyl)-1H-pyrazole-4-sulfonamide (Intermediate A9, Step A) (1.83 g, 4.72 mmol) in NMP (30 mL) at 0° C. was added NaH (60% dispersion in mineral oil) (472 mg, 11.8 mmol) portion-wise. The mixture was stirred at 0° C. for 10 min until effervescence had ceased. Methyl 2,4-dibromo-butanoate (1.00 mL, 7.08 mmol) was then added and the reaction allowed to warm to RT whilst stirring over 20 h. The mixture was then quenched with water (10 mL) and partitioned between brine (300 mL) and MTBE (100 mL). The aqueous layer was re-extracted with MTBE (100 mL) and EtOAc (100 mL). The organic layers were combined, dried (MgSO₄), filtered and concentrated in vacuo. The crude product was purified by FC (0-80% EtOAc/isohexane) to afford the title compound (0.91 g, 1.7 mmol, 37%) as a white solid.

LCMS m/z 486.4 (M+H)⁺ (ES⁺).

¹H NMR (DMSO-d₆) δ 8.47 (s, 1H), 7.84 (s, 1H), 7.07 (d, J=8.5 Hz, 4H), 6.85-6.77 (m, 4H), 4.16 (s, 4H), 3.72 (s, 6H), 3.64 (s, 3H), 1.77-1.73 (m, 2H), 1.69-1.65 (m, 2H).

### Step B: 1-(1-(hydroxymethyl)cyclopropyl)-N,N-bis(4-methoxybenzyl)-1H-pyrazole-4-sulfonamide

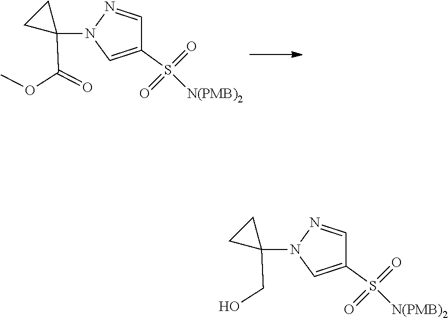

Prepared according to the general procedure of 1-(1-hydroxy-2-methylpropan-2-yl)-N,N-bis(4-methoxybenzyl)-3-methyl-1H-pyrazole-4-sulfonamide (Intermediate A15, Step C) from methyl 1-(4-(N,N-bis(4-methoxybenzyl)sulfamoyl)-1H-pyrazol-1-yl)-cyclopropane-1-carboxylate and lithium borohydride to afford the title compound (0.95 g, 94%) as a thick colourless oil.

LCMS m/z 458.5 (M+H)⁺ (ES⁺).

¹H NMR (DMSO-d₆) δ 8.20 (s, 1H), 7.78 (s, 1H), 7.06 (d, J=8.4 Hz, 4H), 6.81 (d, J=8.4 Hz, 4H), 5.05 (t, J=5.7 Hz, 1H), 4.14 (s, 4H), 3.71 (s, 6H), 3.64 (d, J=5.6 Hz, 2H), 1.16-1.13 (m, 2H), 1.06-1.02 (m, 2H).

### Intermediate A21: 1-(1-hydroxy-2-methylpropan-2-yl)-N,N-bis(4-methoxybenzyl)-1H-imidazole-4-sulfonamide

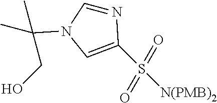

### Step A: methyl 2-(4-(N,N-bis(4-methoxybenzyl)sulfamoyl)-1H-imidazol-1-yl)-2-methylpropanoate

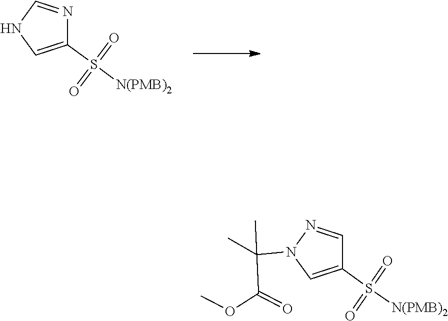

Prepared according to the general procedure of methyl 2-(4-(N,N-bis(4-methoxy-benzyl)sulfamoyl)-1H-pyrazol-1-yl)-2-methylpropanoate (Intermediate A9, Step B) from N,N-bis(4-methoxybenzyl)-1H-imidazole-4-sulfonamide and methyl 2-bromo-2-methylpropanoate to afford the title compound (1.43 g, 90%) as a colourless oil.

LCMS m/z 488.3 (M+H)⁺ (ES⁺).

¹H NMR (DMSO-d₆) δ 8.06 (d, J=1.4 Hz, 1H), 7.97 (d, J=1.4 Hz, 1H), 7.09-6.97 (m, 4H), 6.85-6.76 (m, 4H), 4.21 (s, 4H), 3.71 (s, 6H), 3.69 (s, 3H), 1.81 (s, 6H).

### Step B: 1-(1-hydroxy-2-methylpropan-2-yl)-N,N-bis(4-methoxybenzyl)-1H-imidazole-4-sulfonamide

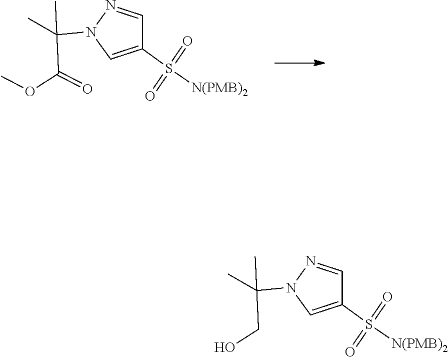

To a mixture of methyl 2-(4-(N,N-bis(4-methoxybenzyl)sulfamoyl)-1H-imidazol-1-yl)-2-methylpropanoate (1.4 g, 2.6 mmol) and THF (20 mL) was added LiBH₄ (4 M in THF) (3 mL, 13 mmol) at 0° C. The reaction mixture was left to stir at RT for 48 h. The reaction mixture was quenched with sat aq NH₄Cl (100 mL), the product was extracted with EtOAc (3×30 mL), the combined organic extracts were passed through a phase separator and concentrated in vacuo to give the crude material as a colourless gum. The crude product was purified by FC (50-100% EtOAc/isohexane) to afford the title compound (840 mg, 67%) as a white solid.

LCMS m/z 460.4 (M+H)⁺ (ES⁺).

¹H NMR (DMSO-d₆) δ 7.93 (s, 1H), 7.87 (s, 1H), 7.03 (d, J=8.1 Hz, 4H), 6.80 (d, J=8.9 Hz, 4H), 5.24 (t, J=5.4 Hz, 1H), 4.20 (s, 4H), 3.71 (s, 6H), 3.53 (d, J=5.4 Hz, 2H), 1.47 (s, 6H).

### Intermediate A22: 2-(1-hydroxy-2-methylpropan-2-yl)-N,N-bis(4-methoxybenzyl)-2H-1,2,3-triazole-4-sulfonamide

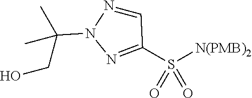

### Step A: methyl 2-(4-(benzylthio)-2H-1,2,3-triazol-2-yl)-2-methylpropanoate

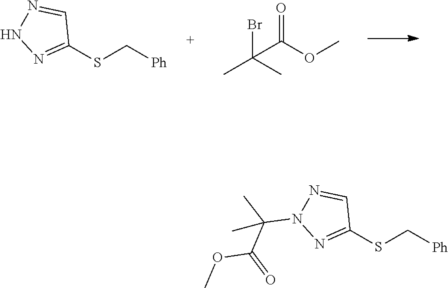

Methyl 2-bromo-2-methylpropanoate (4.1 mL, 32 mmol) was added to a stirred suspension of 4-(benzylthio)-2H-1,2,3-triazole (5.00 g, 93 wt %, 24 mmol) and potassium carbonate (10 g, 73 mmol) in DMF (100 mL) at RT. The mixture was then heated to 70° C. for 3 days. The reaction mixture was left to cool to RT and partitioned between brine (1 L) and MTBE (300 mL). The organic layer was collected, dried (MgSO₄), filtered and concentrated to dryness to give a yellow oil. The crude product was purified by FC (0-50% EtOAc/isohexane) to afford the title compound (3.15 g, 44%) as a thick colourless oil.

LCMS m/z 292.2 (M+H)⁺ (ES⁺).

¹H NMR (DMSO-d₆) δ 7.77 (s, 1H), 7.33-7.16 (m, 5H), 4.19 (s, 2H), 3.63 (s, 3H), 1.81 (s, 6H).

### Step B: methyl 2-(4-(N,N-bis(4-methoxybenzyl)sulfamoyl)-2H-1,2,3-triazol-2-yl)-2-methylpropanoate

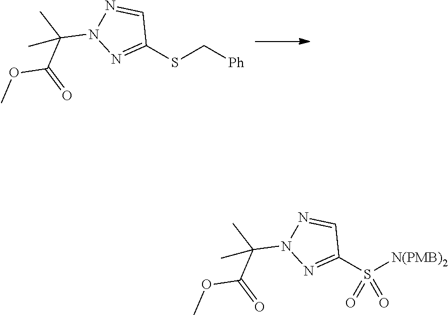

NCS (5-77 g, 43.2 mmol) was added to a solution of methyl 2-(4-(benzylthio)-2H-1,2,3-triazol-2-yl)-2-methylpropanoate (3.15 g, 10.8 mmol) in AcOH (40 mL) and water (20 mL). The mixture was stirred for 4 h then partitioned between DCM (200 mL) and sat aq NaHCO₃ (400 mL), dried (MgSO₄) and filtered. bis(4-Methoxybenzyl)amine (2.78 g, 10.8 mmol) and TEA (2.19 g, 3.01 mL, 21.6 mmol) were then added and the mixture was stirred at RT for 20 h. The mixture was concentrated to ˜100 mL and poured onto 1 M aq HCl (300 mL). The mixture was filtered and the organic layer was separated. The aqueous layer was extracted with DCM (2×100 mL) and the combined organic layers were dried (MgSO₄), filtered and concentrated to dryness to give a yellow oil. The crude product was purified by FC (0-50% EtOAc/isohexane) to afford the title compound (0.58 g, 8%) as a thick yellow oil.

LCMS m/z 511.4 (M+Na)⁺ (ES⁺).

¹H NMR (DMSO-d₆) δ 8.32 (s, 1H), 7.10-7.02 (m, 4H), 6.87-6.80 (m, 4H), 4.25 (s, 4H), 3.72 (s, 6H), 3.67 (s, 3H), 1.87 (s, 6H).

### Step C: 2-(1-hydroxy-2-methylpropan-2-yl)-N,N-bis(4-methoxybenzyl)-2H-1,2,3-triazole-4-sulfonamide

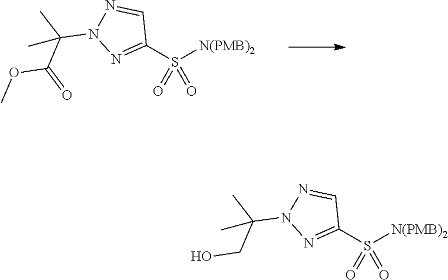

Prepared according to the general procedure of 1-(1-hydroxy-2-methylpropan-2-yl)-N,N-bis(4-methoxybenzyl)-1H-imidazole-4-sulfonamide (Intermediate A21, Step B) from methyl 2-(4-(N,N-bis(4-methoxybenzyl)sulfamoyl)-2H-1,2,3-triazol-2-yl)-2-methylpropanoate and lithium borohydride to afford the title compound (0.38 g, 98%) as a white solid.

LCMS m/z 483.3 (M+Na)⁺ (ES⁺).

¹H NMR (DMSO-d₆) δ 8.21 (s, 1H), 7.05 (d, J=8.4 Hz, 4H), 6.83 (d, J=8.3 Hz, 4H), 5.13 (t, J=5.7 Hz, 1H), 4.24 (s, 4H), 3.77-3.65 (m, 8H), 1.56 (s, 6H).

### Intermediate A23: 1-(1-hydroxy-2-methylpropan-2-yl)-N,N-bis(4-methoxybenzyl)-1H-pyrazolo[3,4-b]pyridine-3-sulfonamide

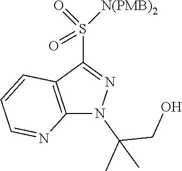

### Step A: methyl 2-(3-bromo-1H-pyrazolo[3,4-b]pyridin-1-yl)-2-methylpropanoate

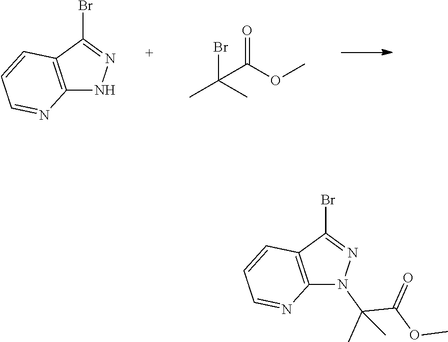

3-Bromo-1H-pyrazolo[3,4-b]pyridine (2.50 g, 12.6 mmol) and potassium carbonate (5.23 g, 37.9 mmol) were suspended in dry DMF (75 mL). Methyl 2-bromo-2-methyl-propanoate (2.12 mL, 16.4 mmol) was added and the mixture was warmed to 80° C. for 16 h. The reaction mixture was cooled to RT, diluted with EtOAc (100 mL) and water (100 mL) and poured on to brine (500 mL). The organics were separated and the aqueous re-extracted with EtOAc (2×100 mL). The organics were combined, dried (MgSO₄) and concentrated in vacuo. The crude product was purified by FC (0-30% EtOAc/isohexane) to afford the title compound (1.44 g, 38%) as a colourless oil.

LCMS m/z 298.3/300.3 (M+H)⁺ (ES⁺).

¹H NMR (DMSO-d₆) δ 8.63 (dd, J=4.5, 1.5 Hz, 1H), 8.15 (dd, J=8.1, 1.6 Hz, 1H), 7.36 (dd, J=8.1, 4.5 Hz, 1H), 3.61 (s, 3H), 1.92 (s, 6H).

### Step B: methyl 2-(3-(benzylthio)-1H-pyrazolo[3,4-b]pyridin-1-yl)-2-methylpropanoate

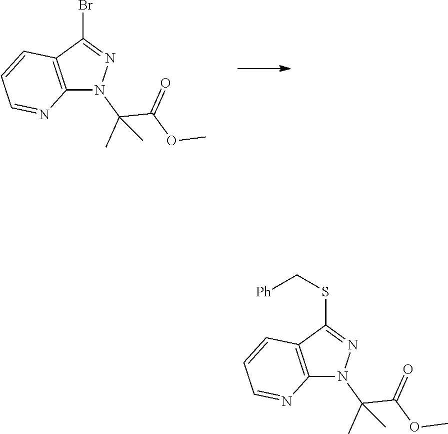 [SMILES: COC(=O)C(C)(C)n1nc(Br)c2cccnc21 ; COC(=O)C(C)(C)n1nc(SCc2ccccc2)c2cccnc21]

Prepared according to the general procedure of methyl 2-(3-(benzylthio)phenyl)acetate (Intermediate A17, Step A) from methyl 2-(3-bromo-1H-pyrazolo[3,4-b]pyridin-1-yl)-2-methylpropanoate and benzyl mercaptan to afford the title compound (1.13 g, 83%) as a thick yellow oil.

LCMS m/z 342.3 (M+H)⁺ (ES⁺).

¹H NMR (DMSO-d₆) δ 8.51 (dd, J=4.5, 1.5 Hz, 1H), 8.03 (dd, J=8.1, 1.5 Hz, 1H), 7.36-7.10 (m, 6H), 4.31 (s, 2H), 3.59 (s, 3H), 1.88 (s, 6H).

### Step C: methyl 2-(3-(N,N-bis(4-methoxybenzyl)sulfamoyl)-1H-pyrazolo[3,4-b]pyridin-1-yl)-2-methylpropanoate

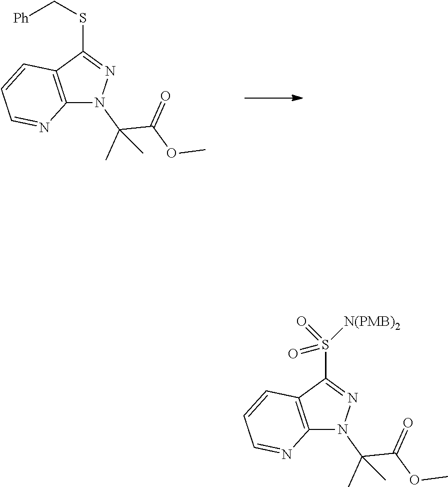

Prepared according to the general procedure of methyl 2-(3-(N,N-bis(4-methoxy-benzyl)sulfamoyl)phenyl)acetate (Intermediate A17, Step B) from methyl 2-(3-(benzylthio)-1H-pyrazolo[3,4-b]pyridin-1-yl)-2-methylpropanoate to afford the title compound (1.38 g, 68%) as a thick pale yellow oil.

LCMS m/z 539.5 (M+H)⁺ (ES⁺).

¹H NMR (DMSO-d₆) δ 8.67 (dd, J=4.5, 1.5 Hz, 1H), 8.37 (dd, J=8.2, 1.5 Hz, 1H), 7.44 (dd, J=8.2, 4.5 Hz, 1H), 7.05 (d, J=8.3 Hz, 4H), 6.75 (d, J=8.4 Hz, 4H), 4.36 (s, 4H), 3.68 (s, 6H), 3.64 (s, 3H), 1.91 (s, 6H).

### Step D: 1-(1-hydroxy-2-methylpropan-2-yl)-N,N-bis(4-methoxybenzyl)-1H-pyrazolo-[3,4-b]pyridine-3-sulfonamide

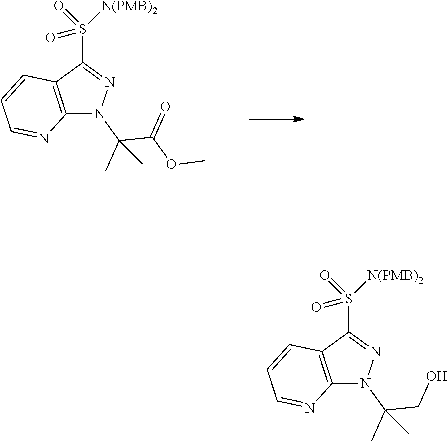

Prepared according to the general procedure of 1-(1-hydroxy-2-methylpropan-2-yl)-N,N-bis(4-methoxybenzyl)-1H-imidazole-4-sulfonamide (Intermediate A21, Step B) from methyl 2-(3-(N,N-bis(4-methoxybenzyl)sulfamoyl)-1H-pyrazolo[3,4-b]pyridin-1-yl)-2-methylpropanoate and lithium borohydride to afford the title compound (1.13 g, 84%) as a white solid.

LCMS m/z 511.3 (M+H)⁺ (ES⁺).

¹H NMR (DMSO-d₆) δ 8.67 (dd, J=4.5, 1.6 Hz, 1H), 8.35 (dd, J=8.2, 1.6 Hz, 1H), 7.41 (dd, J=8.2, 4.4 Hz, 1H), 7.07-7.00 (m, 4H), 6.78-6.71 (m, 4H), 4.98 (t, J=5.8 Hz, 1H), 4.33 (s, 4H), 4.02 (d, J=5.9 Hz, 2H), 3.68 (s, 6H), 1.73 (s, 6H).

### Intermediate A24: 4-fluoro-1-(3-(hydroxymethyl)pyridin-2-yl)-N,N-bis(4-methoxy-benzyl)-1H-pyrazole-3-sulfonamide

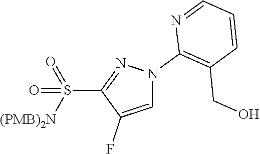

### Step A: 4-fluoro-1-(3-formylpyridin-2-yl)-N,N-bis(4-methoxybenzyl)-1H-pyrazole-3-sulfonamide

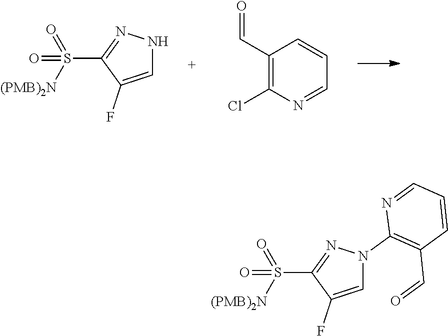

4-fluoro-N,N-bis(4-methoxybenzyl)-1H-pyrazole-3-sulfonamide (Intermediate A4, Step E) (250 mg, 617 μmol), 2-chloronicotinaldehyde (96.0 mg, 678 μmol), 18-crown-6 (8.15 mg, 30.8 μmol), KI (5.12 mg, 30.8 μmol) and K₂CO₃ (256 mg, 1.85 mmol) were taken up in MeCN (6 mL), heated to 70° C. and stirred for 18 h. This experiment was carried out twice. The two reaction mixtures were combined and the mixture was diluted with water (25 mL) and transferred into a separatory funnel. The aqueous layer was extracted with EtOAc (3×10 mL). The combined organic layers were collected, dried over MgSO₄, filtered and concentrated in vacuo. The crude product was purified by FC (0-100% EtOAc/isohexane) to afford the title compound (170 mg, 22%) as a sticky colourless oil.

¹H NMR (CDCl₃) δ 10.27 (d, J=0.8 Hz, 1H), 8.62 (dd, J=4.7, 1.8 Hz, 1H), 8.45 (d, J=4.8 Hz, 1H), 8.34-8.26 (m, 1H), 7.46 (ddd, J=7.7, 4.7, 0.8 Hz, 1H), 7.13 (d, J=8.6 Hz, 4H), 6.75 (d, J=8.7 Hz, 4H), 4.42 (s, 4H), 3.73 (s, 6H).

### Step B: 4-fluoro-1-(3-(hydroxymethyl)pyridin-2-yl)-N,N-bis(4-methoxybenzyl)-1H-pyrazole-3-sulfonamide

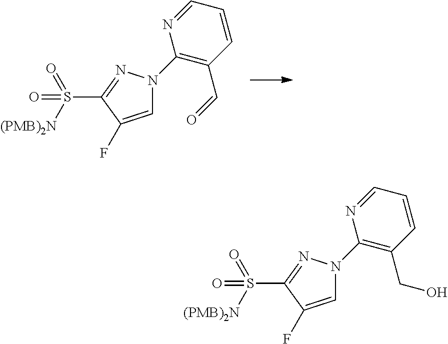

Prepared according to the general procedure of 1-(1-hydroxy-2-methylpropan-2-yl)-N,N-bis(4-methoxybenzyl)-1H-imidazole-4-sulfonamide (Intermediate A21, Step B) from 4-fluoro-1-(3-formylpyridin-2-yl)-N,N-bis(4-methoxybenzyl)-1H-pyrazole-3-sulfonamide and lithium borohydride to afford the title compound (0.20 g, 76%) as a white solid.

LCMS m/z 535.3 (M+Na)⁺ (ES⁺).

¹H NMR (DMSO-d₆) δ 8.79 (d, J=4.4 Hz, 1H), 8.47 (dd, J=4.8, 1.8 Hz, 1H), 8.29-8.21 (m, 1H), 7.60 (dd, J=7.7, 4.7 Hz, 1H), 7.18-7.04 (m, 4H), 6.83-6.76 (m, 4H), 5.51 (t, J=5.4 Hz, 1H), 4.72 (d, J=5.7 Hz, 2H), 4.36 (s, 4H), 3.68 (s, 6H).

### Intermediate A25: 3-(N,N-bis(4-methoxybenzyl)sulfamoyl)-4-fluoro-1-(1-hydroxy-2-methylpropan-2-yl)-1H-pyrazole-5-carboxylic acid

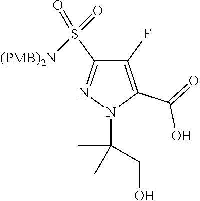

4-Fluoro-1-(1-hydroxy-2-methylpropan-2-yl)-N,N-bis(4-methoxybenzyl)-1H-pyrazole-3-sulfonamide (Intermediate A4) (1.0 g, 1.8 mmol) was dissolved in THF (20 mL) and cooled to −78° C. nBuLi (2.5 M in hexane) (1.6 mL, 4.1 mmol) was added drop-wise, and stirred for 5 min. CO₂ gas was bubbled through the reaction mixture for 5 min and stirred at −78° C. for 10 min, and then allowed to warm to RT and stirred for 1 h. The reaction was quenched with slow addition of sat aq NH₄Cl (10 mL), extracted with EtOAc (2×25 mL), dried (phase separator) and concentrated in vacuo. The crude product was purified by FC (0-10% MeOH/DCM) to afford the title compound (425 mg, 42%) as a colourless oil.

LCMS m/z 544.4 (M+Na)⁺ (ES⁺).

¹H NMR (DMSO-d₆) δ 7.16-7.09 (m, 4H), 6.89-6.81 (m, 4H), 4.61 (s, 2H), 4.33 (s, 4H), 3.73 (s, 6H), 1.47 (s, 6H). Two exchangeable protons not observed.

### Intermediate A26: 1-(1-hydroxy-2-methylpropan-2-yl)-N,N-bis(4-methoxybenzyl)-4,5,6,7-tetrahydro-1H-pyrazolo[3,4-c]pyridine-3-sulfonamide

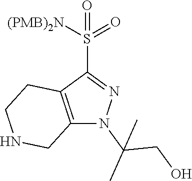

### Step A: methyl 2-(3-bromo-1H-pyrazolo[3,4-c]pyridin-1-yl)-2-methylpropanoate

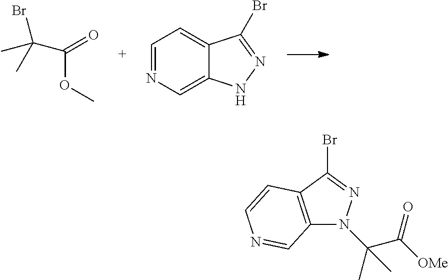

3-bromo-1H-pyrazolo[3,4-c]pyridine (2.50 g, 12.6 mmol) and potassium carbonate (5.23 g, 37.9 mmol) were dissolved in DMF (77 mL). Methyl 2-bromo-2-methyl-propanoate (2.97 g, 16.4 mmol) was added dropwise to the solution which was heated to 80° C. and stirred for 18 h. The solution was diluted with EtOAc (100 mL) and transferred to a separating funnel then washed with a 3:1 brine/water solution (400 mL). The aqueous was washed twice with EtOAc (2×100 mL) and the combined organic layers were washed with brine (200 mL), dried with MgSO₄ and concentrated in vacuo. The crude product was purified by FC (0-100% EtOAc/isohexane) to afford the title compound (2.96 g, 75%) as a thick yellow oil.

LCMS m/z 298.2/300.2 (M+H)⁺ (ES⁺).

¹H NMR (CDCl₃) δ 8.85 (s, 1H), 8.39 (d, J=5.6 Hz, 1H), 7.53 (dd, J=5.6, 1.2 Hz, 1H), 3.74 (d, J=1.0 Hz, 3H), 2.00 (s, 6H).

### Step B: methyl 2-(3-(benzylthio)-1H-pyrazolo[3,4-c]pyridin-1-yl)-2-methylpropanoate

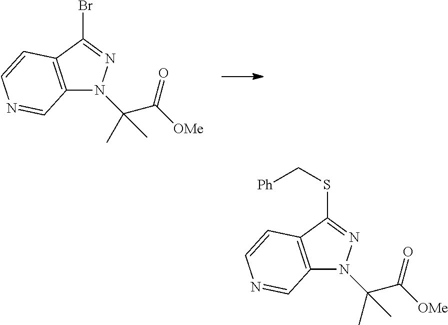 [SMILES: COC(=O)C(C)(C)n1nc(Br)c2ccncc21 ; COC(=O)C(C)(C)n1nc(SCc2ccccc2)c2ccncc21]

Prepared according to the general procedure of methyl 2-(3-(benzylthio)phenyl)acetate (Intermediate A17, Step A) from methyl 2-(3-bromo-1H-pyrazolo[3,4-c]pyridin-1-yl)-2-methylpropanoate and benzyl mercaptan to afford the title compound (3.0 g, 94%) as a thick yellow oil.

LCMS m/z 342.3 (M+H)⁺ (ES⁺).

¹H NMR (DMSO-d₆) δ 8.97 (d, J=1.3 Hz, 1H), 8.26 (d, J=5.5 Hz, 1H), 7.57 (dd, J=5.6, 1.2 Hz, 1H), 7.28-7.17 (m, 5H), 4.30 (s, 2H), 3.68 (s, 3H), 1.93 (s, 6H).

### Step C: methyl 2-(3-(N,N-bis(4-methoxybenzyl)sulfamoyl)-1H-pyrazolo[3,4-c]pyridin-1-yl)-2-methylpropanoate

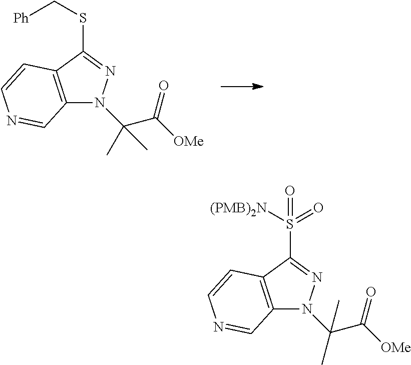

Prepared according to the general procedure of methyl 2-(3-(N,N-bis(4-methoxy-benzyl)sulfamoyl)phenyl)acetate (Intermediate A17, Step B) from methyl 2-(3-(benzylthio)-1H-pyrazolo[3,4-c]pyridin-1-yl)-2-methylpropanoate to afford the title compound (3.47 g, 59%) as a thick pale yellow oil.

LCMS m/z 539.4 (M+H)⁺ (ES⁺).

¹H NMR (DMSO-d₆) δ 9.16 (d, J=5.3 Hz, 1H), 8.45 (t, J=5.4 Hz, 1H), 7.92 (d, J=5.6 Hz, 1H), 7.05 (d, J=8.3 Hz, 4H), 6.80-6.72 (m, 4H), 4.36 (d, J=4.6 Hz, 4H), 3.75 (d, J=2.7 Hz, 3H), 3.69 (s, 6H), 1.96 (s, 6H).

### Step D: 1-(1-hydroxy-2-methylpropan-2-yl)-N,N-bis(4-methoxybenzyl)-4,5,6,7-tetrahydro-1H-pyrazolo[3,4-c]pyridine-3-sulfonamide

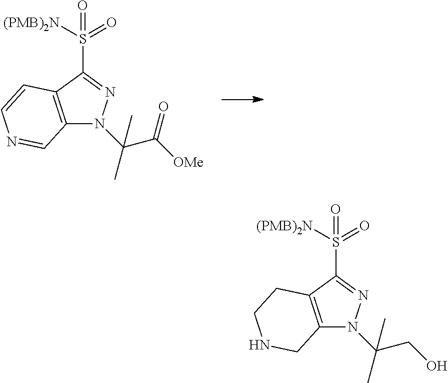

Prepared according to the general procedure of 1-(1-hydroxy-2-methylpropan-2-yl)-N,N-bis(4-methoxybenzyl)-1H-imidazole-4-sulfonamide (Intermediate A21, Step B) from methyl 2-(3-(N,N-bis(4-methoxybenzyl)sulfamoyl)-1H-pyrazolo[3,4-c]pyridin-1-yl)-2-methylpropanoate and lithium borohydride to afford the title compound (355 mg, 7%) as a white solid.

LCMS m/z 515.5 (M+H)⁺ (ES⁺).

¹H NMR (DMSO-d₆) δ 7.02 (d, J=8.6 Hz, 4H), 6.81 (d, J=8.7 Hz, 4H), 5.08 (br s, 1H), 4.21 (s, 4H), 4.01 (s, 2H), 3.72 (s, 6H), 3.57 (s, 2H), 2.76 (t, J=5.7 Hz, 2H), 2.54 (t, J=5.8 Hz, 2H), 1.47 (s, 6H). One exchangeable proton not observed.

### Intermediate A27: potassium 3-(N,N-bis(4-methoxybenzyl)sulfamoyl)-1-(1-hydroxy-2-methylpropan-2-yl)-1H-pyrazole-5-carboxylate

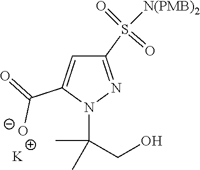

### Step A: N,N-bis(4-methoxybenzyl)-7,7-dimethyl-4-oxo-6,7-dihydro-4H-pyrazolo[5,1-c][1,4]oxazine-2-sulfonamide

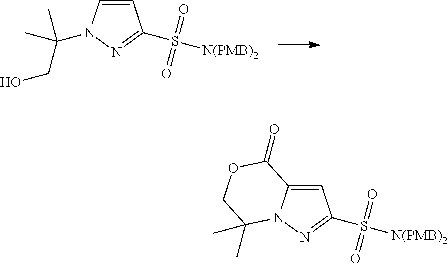

2.5 M butyllithium in hexanes (5.76 mL, 14.4 mmol) was added dropwise to a solution of 1-(1-hydroxy-2-methylpropan-2-yl)-N,N-bis(4-methoxybenzyl)-1H-pyrazole-3-sulfonamide (Intermediate A1) (3.23 g, 7.03 mmol) in 9:1 THF:DMPU (50 mL) cooled to −78° C. The mixture was stirred at −78° C. for 10 min and CO₂ gas was bubbled in for a further 5 min. The mixture was then stirred for 5 min and left to warm to RT over 1 h. Acetic acid (30 mL) was added and the mixture was stirred at RT overnight. The mixture was poured onto sat aq NaHCO₃ (200 mL). The mixture was extracted with EtOAc (3×30 mL) and the combined organic layers were dried (MgSO₄), filtered and concentrated to dryness. The crude product was purified by FC (0-100% EtOAc/isohexane) to afford the title compound (1.11 g, 23%) as a thick colourless oil.

¹H NMR (DMSO-d₆) δ 7.33 (s, 1H), 7.10-7.03 (m, 4H), 6.87-6.81 (m, 4H), 4.63 (s, 2H), 4.27 (s, 4H), 3.72 (s, 6H), 1.51 (s, 6H).

### Step B: potassium 3-(N,N-bis(4-methoxybenzyl)sulfamoyl)-1-(1-hydroxy-2-methyl-propan-2-yl)-1H-pyrazole-5-carboxylate

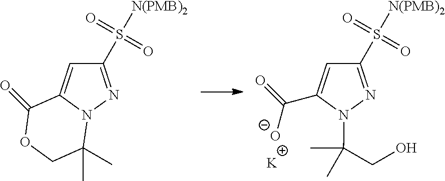

To a stirred solution of N,N-bis(4-methoxybenzyl)-7,7-dimethyl-4-oxo-6,7-dihydro-4H-pyrazolo[5,1-c][1,4]oxazine-2-sulfonamide (1.11 g, 2.29 mmol) in dry THF (25 mL) under a N₂ atmosphere at RT was added potassium trimethylsilanolate (587 mg, 4.57 mmol). The reaction mixture was stirred for 18 h. The reaction mixture was concentrated to dryness, dissolved in THF (10 mL) and diluted with MTBE (100 mL). The supernatant was decanted and the pale yellow gummy residue was washed with MTBE (50 mL). The supernatant was decanted and the vessel concentrated to dryness to give the title compound (1.17 g, 85%) as a pale yellow solid.

LCMS m/z 526.3 (M+H+Na−K)⁺ (ES⁺); 502.3 (M−K)⁻ (ES⁻).

¹H NMR (DMSO-d₆) δ 7.07-6.93 (m, 4H), 6.87-6.77 (m, 4H), 6.45 (s, 1H), 4.18 (s, 4H), 3.72 (s, 6H), 3.69 (s, 2H), 3.21-3.16 (m, 1H), 1.58 (s, 6H).

### Intermediate A28: 1-(5-hydroxypentyl)-N,N-bis(4-methoxybenzyl)-1H-pyrazole-5-sulfonamide

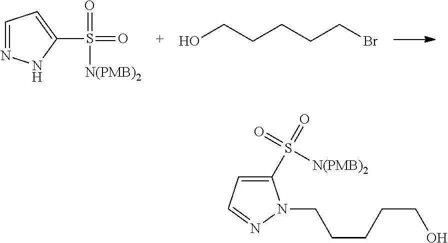

To a solution of N,N-bis(4-methoxybenzyl)-1H-pyrazole-3-sulfonamide (Intermediate A1, Step C) (4 g, 10.3 mmol) and K₂CO₃ (4.28 g, 31.0 mmol) in MeCN (50 mL) was added 5-bromopentan-1-ol (5.17 g, 31.0 mmol) at 20° C. Then the mixture was stirred at 70° C. for 12 h. The mixture was poured into water (300 mL) and extracted with DCM (2×200 mL). The combined organic layers were washed with brine (300 mL), dried over anhydrous sodium sulfate, filtered and concentrated under reduced pressure to give the crude product as a mixture of regioisomers. The residue was purified by FC (PE:EtOAc, 3:1 to 1:1) to give the title compound as the minor regioisomer (800 mg, 16-36% yield) as a yellow oil.

LCMS: m/z 512.2 (M+K)+(ES⁺).

¹H NMR (CDCl₃) δ 7.50 (d, 1H), 7.00-6.97 (m, 4H), 6.85-6.82 (m, 4H), 6.57 (d, 1H), 4.36-4.30 (m, 6H), 3.81 (s, 6H), 3.64 (t, 2H), 2.05-1.92 (m, 2H), 1.61-1.57 (m, 2H), 1.43-1.39 (m, 2H). One exchangeable proton not observed.

### Intermediate A29: 3-(N,N-bis(4-methoxybenzyl)sulfamoyl)-1-(1-hydroxy-2-methylpropan-2-yl)-N,N-dimethyl-1H-pyrazole-4-carboxamide

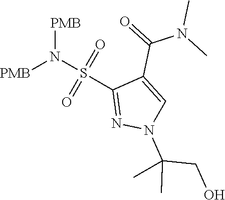

### Step A: ethyl 3-amino-1H-pyrazole-4-carboxylate

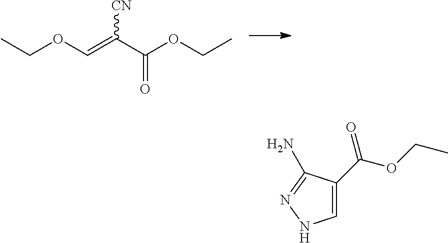

To a solution of ethyl 2-cyano-3-ethoxyacrylate (20 g, 118 mmol) in EtOH (200 mL) was added N₂H₄.H₂O (6.12 g, 120 mmol, 98% purity). The reaction solution was stirred at 20° C. for 3 h, then concentrated under reduced pressure. The residue was triturated with MTBE (200 mL) to give the title compound (13.3 g, 72.5% yield) as a yellow solid.

LCMS: m/z 156.1 (M+H)⁺ (ES⁺).

¹H NMR (CDCl₃): δ 9.68 (s, 1H), 7.57 (d, 1H), 4.30 (br s, 2H), 4.23 (q, 2H), 1.32 (t, 3H).

### Step B: ethyl 3-(chlorosulfonyl)-1H-pyrazole-4-carboxylate

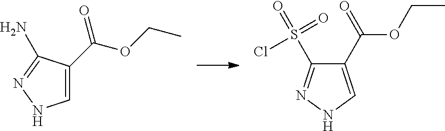

To a solution of conc HCl (22 mL, 36% purity in water) and H₂O (22 mL) was added ethyl 3-amino-1H-pyrazole-4-carboxylate (10.8 g, 69.6 mmol). Then a solution of NaNO₂ (5.04 g, 73.1 mmol) in H₂O (10.8 mL) was added slowly to the mixture keeping the temperature below 3° C. The mixture was stirred at 0° C. for 1 h to give a diazonium salt solution. SO₂ gas was bubbled into AcOH (80 mL) at 0-5° C. for 0.5 h, then CuCl₂ (4.68 g, 34.8 mmol) was added into the sulfur dioxide solution. The above diazonium salt solution was added dropwise into the saturated sulphur dioxide solution at 0° C., then the mixture was stirred at 20° C. for 0.5 h. Water (200 mL) and DCM (200 mL) were added into the reaction mixture and the layers were separated. The aqueous phase was extracted with DCM (200 mL×2). The combined organic phases were dried over anhydrous Na₂SO₄, filtered and concentrated in vacuum. The residue was purified by FC (PE:EtOAc, 20:1 to 1:1) to give the title compound (5 g, 24.8% yield) as a yellow solid.

¹H NMR (DMSO-d₆): δ 7.76 (s, 1H), 4.13-4.15 (m, 2H), 1.23 (t, 3H). One exchangeable proton not observed.

### Step C: ethyl 3-(N,N-bis(4-methoxybenzyl)sulfamoyl)-1H-pyrazole-4-carboxylate

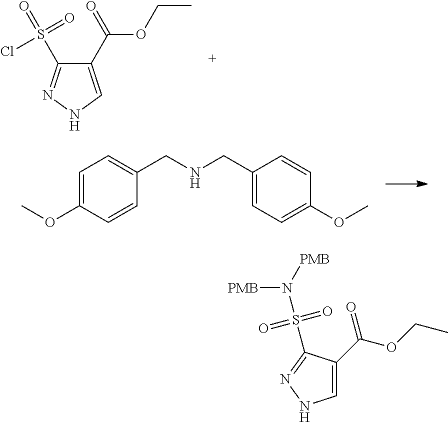

To a solution of 1-(4-methoxyphenyl)-N-[(4-methoxyphenyl)methyl]methanamine (4.85 g, 18.9 mmol) in DCM (50 mL) was added TEA (6.36 g, 62.9 mmol), followed by another solution of ethyl 3-(chlorosulfonyl)-1H-pyrazole-4-carboxylate (5.0 g, 17.3 mmol, 82.5% purity) in THF (100 mL). The reaction mixture was stirred at 20° C. for 12 h, then concentrated in vacuum. The residue was purified by reverse phase flash chromatography (0.1% TFA). The eluting phase was adjusted to pH 8 with solid NaHCO₃, and the aqueous phase was extracted with EtOAc (200 mL×3). The combined organic phases were dried over anhydrous Na₂SO₄, filtered and concentrated in vacuum to give the title compound (8.0 g, 97.4% yield) as a yellow solid.

LCMS: m/z 460.1 (M+H)⁺ (ES⁺).

¹H NMR (DMSO-d₆): δ 8.52 (s, 1H), 7.01 (d, 4H), 6.77 (d, 4H), 4.36 (s, 4H), 4.23 (q, 2H), 3.70 (s, 6H), 1.26 (t, 3H). One exchangeable proton not observed.

### Step D: 3-(N,N-bis(4-methoxybenzyl)sulfamoyl)-1H-pyrazole-4-carboxylic acid

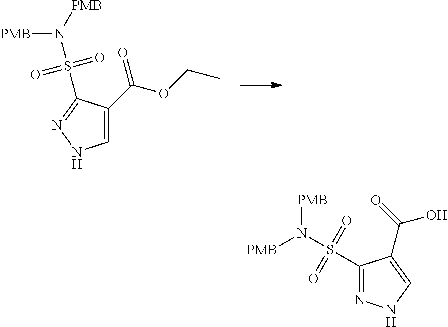

To a solution of ethyl 3-(N,N-bis(4-methoxybenzyl)sulfamoyl)-1H-pyrazole-4-carboxylate (8.0 g, 17.4 mmol) in THF (80 mL) was added a solution of LiOH.H₂O (4 g, 95.3 mmol) in H₂O (80 mL). The reaction mixture was stirred at 65° C. for 12 h, then washed with EtOAc (200 mL×2). The aqueous layer was adjusted to pH 2 with 1 M aq HCl and extracted with EtOAc (200 mL×2). The combined organic phases were washed with brine (200 mL), dried over anhydrous Na₂SO₄, filtered and concentrated in vacuum to give the title compound (7.0 g, 93.2% yield) as a white solid.

LCMS: m/z 454.1 (M+Na)⁺ (ES⁺).

¹H NMR (DMSO-d₆): δ 13.96 (br s, 1H), 12.74 (br s, 1H), 8.47 (s, 1H), 7.02-6.79 (m, 4H), 6.79-6.76 (m, 4H), 4.35 (s, 4H), 3.70 (s, 6H).

### Step E: 3-(N,N-bis(4-methoxybenzyl)sulfamoyl)-N,N-dimethyl-1H-pyrazole-4-carboxamide

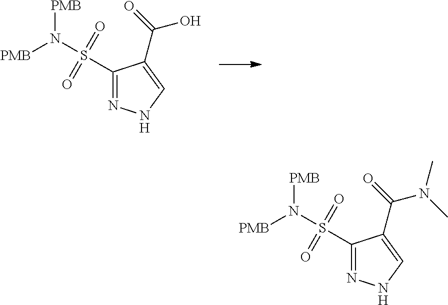

To a solution of 3-(N,N-bis(4-methoxybenzyl)sulfamoyl)-1H-pyrazole-4-carboxylic acid (2 g, 4.64 mmol) and dimethylamine (567 mg, 6.96 mmol, HCl salt) in DMF (30 mL) was added DIPEA (1.50 g, 11.6 mmol) and T₃P (4.43 g, 6.96 mmol, 50% purity in EtOAc) at 0° C. The solution was stirred at 25° C. for 2 h, then quenched with H₂O (80 mL) and extracted with EtOAc (50 mL×2). The organic phases were washed with brine (60 mL×2), dried over anhydrous Na₂SO₄, filtered and concentrated in vacuum. The residue was purified by FC (PE:EtOAc, 1:0 to 0:1) to give the title compound (2.1 g, 98.7% yield) as a colourless oil.

LCMS: m/z 459.3 (M+H)⁺ (ES⁺).

¹H NMR (CDCl₃): δ 13.02 (s, 1H), 7.85 (s, 1H), 7.00 (d, 4H), 6.72 (d, 4H), 4.33 (s, 4H), 3.74 (s, 6H), 3.11 (s, 3H), 2.96 (s, 3H).

### Step F: methyl 2-(3-(N,N-bis(4-methoxybenzyl)sulfamoyl)-4-(dimethylcarbamoyl)-1H-pyrazol-1-yl)-2-methylpropanoate

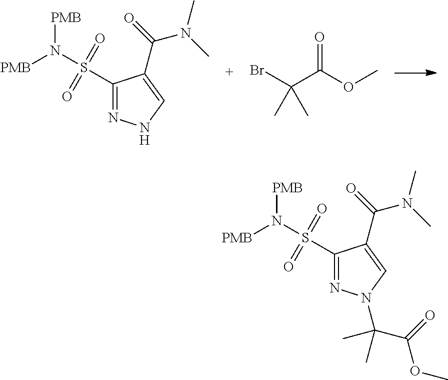

To a solution of 3-(N,N-bis(4-methoxybenzyl)sulfamoyl)-N,N-dimethyl-1H-pyrazole-4-carboxamide (1.5 g, 3.27 mmol) and methyl 2-bromo-2-methylpropanoate (1.18 g, 6.54 mmol) in MeCN (40 mL) was added K₂CO₃ (904 mg, 6.54 mmol) and the mixture was stirred at 65° C. for 1 h. The mixture was filtered and the filtrate was concentrated in vacuum. The residue was purified by FC (PE:EtOAc, 2:1 to 1:2) to give the title compound (1.8 g, 98.5% yield) as a colourless oil.

LCMS: m/z 559.2 (M+H)⁺ (ES⁺).

¹H NMR (CDCl₃): δ 7.70 (s, 1H), 7.07 (d, 4H), 6.77 (d, 4H), 4.31 (s, 4H), 3.78 (s, 6H), 3.70 (s, 3H), 3.11 (s, 3H), 3.03 (s, 3H), 1.63 (s, 6H).

### Step G: 3-(N,N-bis(4-methoxybenzyl)sulfamoyl)-1-(1-hydroxy-2-methylpropan-2-yl)-N,N-dimethyl-1H-pyrazole-4-carboxamide

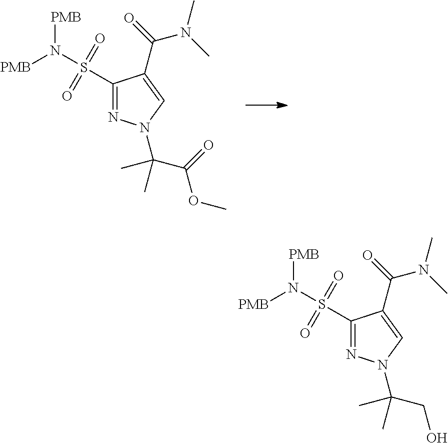

To a solution of methyl 2-(3-(N,N-bis(4-methoxybenzyl)sulfamoyl)-4-(dimethyl-carbamoyl)-1H-pyrazol-1-yl)-2-methylpropanoate (2.3 g, 4.12 mmol) in EtOH (40 mL) was added NaBH₄ (467 mg, 12.4 mmol) in portions at 0° C. The resultant solution was stirred at 25° C. for 2 h, then quenched with 1 M aq HCl to pH 5 at 0° C. under N₂. The mixture was diluted with H₂O (100 mL) and extracted with EtOAc (50 mL×3). The organic phases were washed with brine (100 mL), dried over anhydrous Na₂SO₄, filtered and concentrated in vacuum. The residue was purified by FC (PE:EtOAc, 1:1 to 0:1) to give the title compound (2.15 g, 98.4% yield) as a white solid.

LCMS: m/z 531.1 (M+H)⁺ (ES⁺).

¹H NMR (CDCl₃): δ 7.58 (s, 1H), 7.00 (d, 4H), 6.69 (d, 4H), 4.25 (s, 4H), 3.70-3.67 (m, 8H), 3.02 (s, 3H), 2.92 (s, 3H), 1.44 (s, 6H). One exchangeable proton not observed.

### Intermediate A30: 4-fluoro-1-(1-hydroxy-2-methylpropan-2-yl)-N,N-bis(4-methoxybenzyl)-5-((4-methylpiperazin-1-yl)methyl)-1H-pyrazole-3-sulfonamide

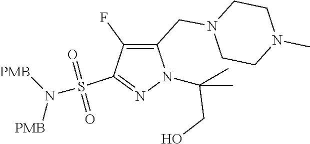

### Step A: 5-(N,N-bis(4-methoxybenzyl)sulfamoyl)-4-fluoro-1H-pyrazole-3-carboxylic acid

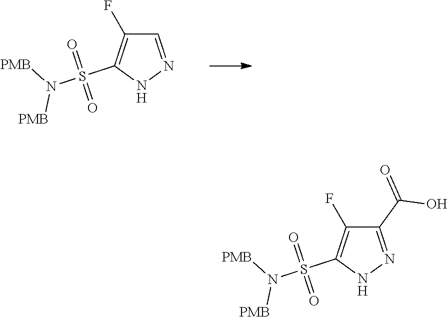

To a solution of 4-fluoro-N,N-bis(4-methoxybenzyl)-1H-pyrazole-3-sulfonamide (Intermediate A4, Step E) (13.5 g, 33.3 mmol) in THF (200 mL) was added slowly n-BuLi (2.5 M, 26.6 mL) at −70° C. under N₂. The mixture was stirred for 0.5 h, then CO₂ (solid) was added slowly into the reaction mixture. The reaction mixture was stirred at 20° C. for 1 h, then concentrated in vacuum. The residue was purified by reverse phase flash chromatography (water (0.1% NH₃.H₂O)-MeCN) to give the title compound (3 g, 20.1% yield) as a yellow solid.

LCMS: m/z 448.1 (M−H)⁻ (ES⁻).

¹H NMR (DMSO-d₆): δ 7.00 (d, 4H), 6.76 (d, 4H), 4.15 (s, 4H), 3.70 (s, 6H). Two exchangeable protons not observed.

### Step B: 4-fluoro-N,N-bis(4-methoxybenzyl)-3-(4-methylpiperazine-1-carbonyl)-1H-pyrazole-5-sulfonamide

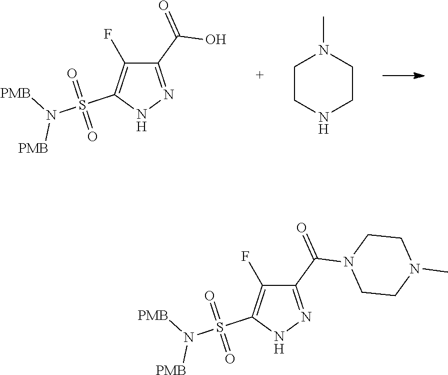

To a solution of 5-(N,N-bis(4-methoxybenzyl)sulfamoyl)-4-fluoro-1H-pyrazole-3-carboxylic acid (500 mg, 1.11 mmol) in DMF (10 mL) was added DIPEA (431 mg, 3.34 mmol) and HATU (635 mg, 1.67 mmol). The mixture was stirred at 20° C. for 10 min, then 1-methylpiperazine (134 mg, 1.33 mmol) was added at 20° C. The reaction mixture was stirred at 20° C. for 12 h, then quenched with H₂O (50 mL) and extracted with EtOAc (50 mL×3). The organic phases were washed with brine (50 mL×2), dried over anhydrous Na₂SO₄, filtered and concentrated in vacuum. The residue was purified by FC (DCM:MeOH, 30:1 to 20:1) to give the title compound (400 mg, 67.6% yield) as a yellow oil.

LCMS: m/z 532.2 (M+H)⁺ (ES⁺).

¹H NMR (CDCl₃): δ 7.08 (d, 4H), 6.78 (d, 4H), 4.38 (s, 4H), 3.79 (s, 6H), 3.73-3.78 (m, 2H), 3.62-3.59 (m, 2H), 2.55-2.51 (m, 4H), 2.40 (s, 3H). One exchangeable proton not observed.

### Step C: methyl 2-(3-(N,N-bis(4-methoxybenzyl)sulfamoyl)-4-fluoro-5-(4-methyl-piperazine-1-carbonyl)-1H-pyrazol-1-yl)-2-methylpropanoate

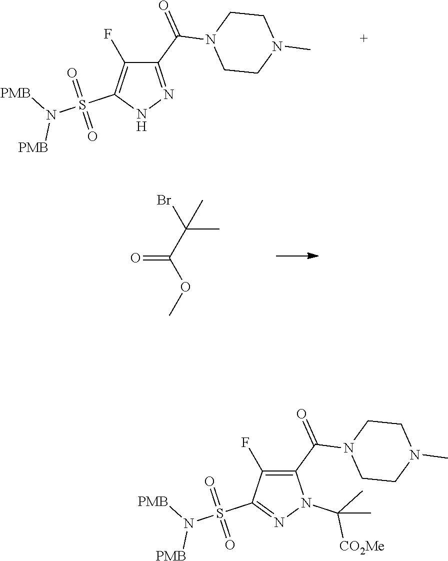

To a solution of 4-fluoro-N,N-bis(4-methoxybenzyl)-3-(4-methylpiperazine-1-carbonyl)-1H-pyrazole-5-sulfonamide (1.5 g, 2.82 mmol) in MeCN (10 mL) was added K₂CO₃ (975 mg, 7.05 mmol) and methyl 2-bromo-2-methylpropanoate (613 mg, 3.39 mmol). The mixture was stirred at 70° C. for 2 h. Additional K₂CO₃ (975 mg, 7.05 mmol) and methyl 2-bromo-2-methylpropanoate (613 mg, 3.39 mmol) were added to the reaction mixture three times until the reaction was completed. The mixture was quenched with H₂O (150 mL) and extracted with EtOAc (200 mL×3). The organic phases were washed with brine (200 mL×2), dried over anhydrous Na₂SO₄, filtered and concentrated in vacuum. The residue was purified by FC (DCM:MeOH, 20:1 to 10:1) to give the title compound (1.5 g, 84.2% yield) as a yellow solid.

LCMS: m/z 632.2 (M+H)⁺ (ES⁺).

¹H NMR (DMSO-d₆): δ 7.11 (d, 4H), 6.83 (dd, 4H), 4.33 (s, 4H), 3.70-3.74 (m, 8H), 3.67 (s, 3H), 3.67-3.64 (m, 2H), 2.38-2.34 (m, 4H), 2.24 (s, 3H), 1.71 (s, 6H).

### Step D: 4-fluoro-1-(1-hydroxy-2-methylpropan-2-yl)-N,N-bis(4-methoxybenzyl)-5-((4-methylpiperazin-1-yl)methyl)-1H-pyrazole-3-sulfonamide

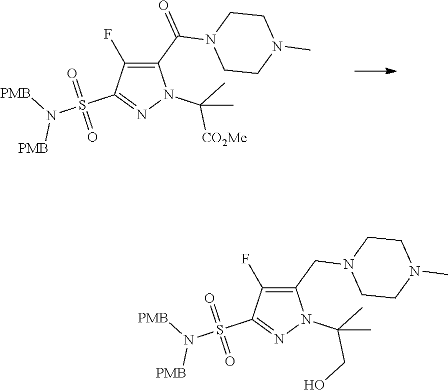

To a solution of methyl 2-(3-(N,N-bis(4-methoxybenzyl)sulfamoyl)-4-fluoro-5-(4-methylpiperazine-1-carbonyl)-1H-pyrazol-1-yl)-2-methylpropanoate (500 mg, 792 μmol) in THF (10 mL) was added dropwise BH₃-Me₂S (10 M, 2.37 mL) at 0° C. under N₂. The mixture was stirred at 20° C. for 0.5 h, then heated to 60° C. and stirred at 60° C. for 3 h. The reaction mixture was cooled to 20° C. and MeOH (20 mL) was added under N₂. The mixture was stirred at 20° C. for 1 h, then heated to 60° C. for 12 h. The mixture was concentrated in vacuum. The residue was purified by reverse phase flash chromatography (water (0.1% TFA)-MeCN) to give the title compound (320 mg, 57.5% yield, TFA salt) as a yellow oil.

LCMS: m/z 590.6 (M−TFA+H)⁺ (ES⁺).

¹H NMR (DMSO-d₆): δ 9.60 (br s, 1H), 7.08 (dd, 4H), 6.83 (d, 4H), 5.13 (br s, 1H), 4.27 (s, 4H), 3.74-3.71 (m, 8H), 3.64 (s, 2H), 3.45-3.43 (m, 2H), 3.04-2.97 (m, 2H), 2.97-2.85 (m, 2H), 2.81 (s, 3H), 2.38-2.30 (m, 2H), 1.55 (s, 6H).

### Intermediate A31: 4-fluoro-1-(1-hydroxy-2-methylpropan-2-yl)-N,N-bis(4-methoxy-benzyl)-5-(4-methylpiperazine-1-carbonyl)-1H-pyrazole-3-sulfonamide

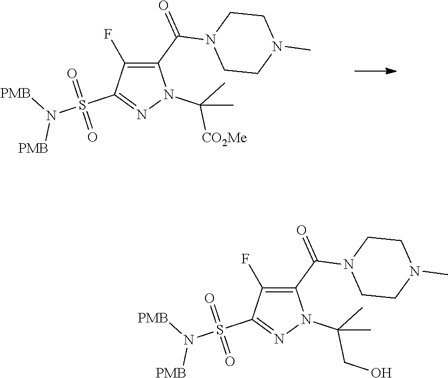

To a solution of methyl 2-(3-(N,N-bis(4-methoxybenzyl)sulfamoyl)-4-fluoro-5-(4-methylpiperazine-1-carbonyl)-1H-pyrazol-1-yl)-2-methylpropanoate (Intermediate A30, Step C) (600 mg, 950 μmol) in THF (15 mL) was added LiAlH₄ (108 mg, 2.85 mmol) in portions at 0° C. The mixture was stirred at 0° C. for 1 h, then quenched with EtOAc (10 mL) and concentrated in vacuum. The residue was purified by reverse phase flash chromatography (water (0.1% TFA)-MeCN) to give the title compound (0.28 g, 48.8% yield, TFA salt) as a yellow solid.

LCMS: m/z 604.5 (M-TFA+H)⁺ (ES⁺).

¹H NMR (DMSO-d₆): δ 7.12 (d, 4H), 6.86 (dd, 4H), 4.61 (s, 2H), 4.35-4.33 (m, 4H), 3.74-3.69 (m, 10H), 3.13-3.07 (m, 2H), 2.86-2.84 (m, 2H), 2.68-2.64 (m, 3H), 1.47 (s, 6H). One exchangeable proton and TFA proton not observed.

### Intermediate A-32: 4-fluoro-1-(1-hydroxy-2-methylpropan-2-yl)-N,N-bis(4-methoxybenzyl)-5-(morpholinomethyl)-1H-pyrazole-3-sulfonamide

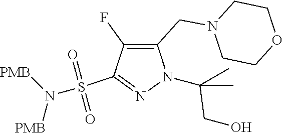

### Step A: 4-fluoro-N,N-bis(4-methoxybenzyl)-5-(morpholine-4-carbonyl)-1H-pyrazole-3-sulfonamide

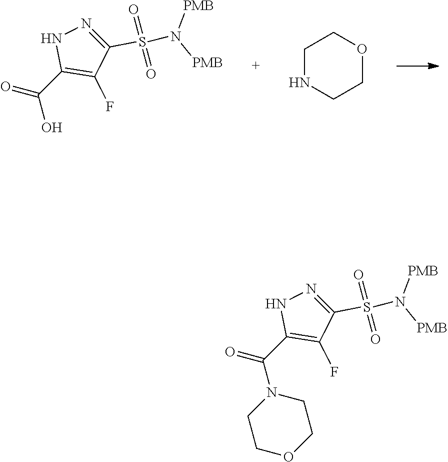

To a solution of 5-(N,N-bis(4-methoxybenzyl)sulfamoyl)-4-fluoro-1H-pyrazole-3-carboxylic acid (Intermediate A30, Step A) (0.6 g, 1.33 mmol) in DMF (5 mL) was added DIPEA (518 mg, 4.00 mmol) and HATU (761 mg, 2.00 mmol). Then morpholine (139 mg, 1.60 mmol) was added to the mixture at 20° C. The reaction mixture was stirred at 20° C. for 12 h, then purified by reverse phase flash chromatography (water (0.1% TFA)-MeCN) to give the title compound (0.45 g, 65.0% yield) as a yellow gum.

LCMS: m/z 519.1 (M+H)⁺ (ES⁺).

¹H NMR (DMSO-d₆): δ 7.07 (d, 4H), 6.81 (d, 4H), 4.30 (s, 4H), 3.71 (s, 6H), 3.67-3.56 (m, 6H), 3.42-3.41 (m, 2H). One exchangeable proton not observed.

### Step B: methyl 2-(3-(N,N-bis(4-methoxybenzyl)sulfamoyl)-4-fluoro-5-(morpholine-4-carbonyl)-1H-pyrazol-1-yl)-2-methylpropanoate

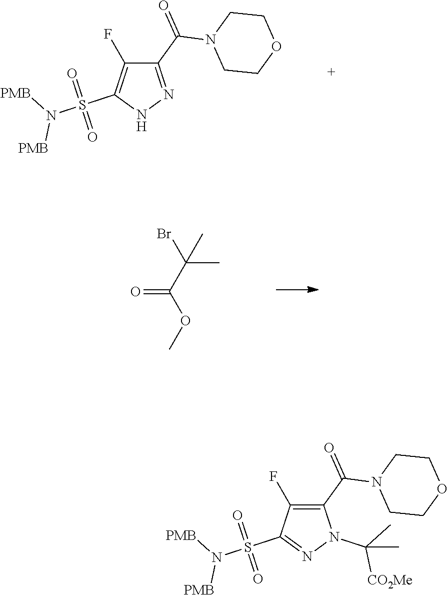

To a solution of 4-fluoro-N,N-bis(4-methoxybenzyl)-5-(morpholine-4-carbonyl)-1H-pyrazole-3-sulfonamide (0.4 g, 771 μmol) in DMF (8 mL) was added Cs₂CO₃ (754 mg, 2.31 mmol) and methyl 2-bromo-2-methylpropanoate (279 mg, 1.54 mmol). The reaction mixture was stirred at 60° C. for 12 h, then water (100 mL) and EtOAc (100 mL) were added and the mixture was separated. The aqueous layer was extracted with EtOAc (100 mL×2). The combined organic phases were dried over anhydrous Na₂SO₄, filtered and concentrated in vacuum. The residue was purified by FC (PE:EtOAc, 1:0 to 1:1) to give the title compound (0.37 g, 78.2% yield) as a yellow oil.

LCMS: m/z 641.1 (M+Na)⁺ (ES⁺).

¹H NMR (CDCl₃): δ 7.11 (dd, 4H), 6.81 (dd, 4H), 4.38 (s, 4H), 3.80 (s, 6H), 3.77 (s, 3H), 3.75-3.73 (m, 4H), 3.69-3.68 (m, 2H), 3.50-3.49 (m, 2H), 1.83 (s, 6H).

### Step C: 4-fluoro-1-(1-hydroxy-2-methylpropan-2-yl)-N,N-bis(4-methoxybenzyl)-5-(morpholinomethyl)-1H-pyrazole-3-sulfonamide

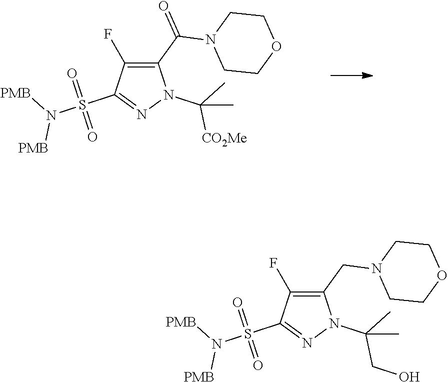

To a solution of methyl 2-(3-(N,N-bis(4-methoxybenzyl)sulfamoyl)-4-fluoro-5-(morpholine-4-carbonyl)-1H-pyrazol-1-yl)-2-methylpropanoate (0.7 g, 1.13 mmol) in THF (7 mL) was added BH₃-Me₂S (10 M, 7 mL) at 0° C. under N₂. The reaction mixture was stirred at 20° C. for 0.5 h, then stirred at 60° C. for 12 h. The reaction mixture was quenched with MeOH (20 mL) at 0° C., then stirred at 75° C. for 2 h. The mixture was concentrated in vacuum. The residue was purified by reverse phase flash chromatography (water (0.1% TFA)-MeCN) to give the title compound (0.62 g, 93.5% yield) as a yellow gum.

LCMS: m/z 577.2 (M+H)⁺ (ES⁺).

¹H NMR (CDCl₃): δ 7.12 (d, 4H), 6.80 (dd, 4H), 5.58 (br s, 1H), 4.40 (s, 4H), 4.34-4.31 (m, 2H), 3.97-3.89 (m, 4H), 3.80 (s, 6H), 3.71 (s, 2H), 3.22-3.30 (m, 4H), 1.67 (s, 6H).

### Intermediate A33: 1-(1-amino-2-methylpropan-2-yl)-N,N-bis(4-methoxybenzyl)-1H-pyrazole-4-sulfonamide

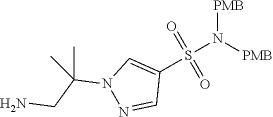

### Step A: 2-(4-(N,N-bis(4-methoxybenzyl)sulfamoyl)-1H-pyrazol-1-yl)-2-methyl-propanamide

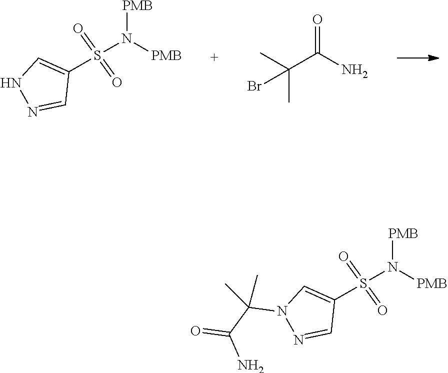

To a solution of N,N-bis(4-methoxybenzyl)-1H-pyrazole-4-sulfonamide (Intermediate A9, Step A) (2 g, 5.16 mmol) in MeCN (30 mL) was added 2-bromo-2-methylpropanamide (1.11 g, 6.69 mmol) and K₂CO₃ (1.43 g, 10.4 mmol). The reaction mixture was stirred at 60° C. for 16 h, then quenched with H₂O (30 mL) and extracted with EtOAc (20 mL×2). The organic phases were washed with brine (30 mL×3), dried over anhydrous Na₂SO₄, filtered and concentrated in vacuum. The residue was purified by FC (PE:EtOAc, 2:3 to 0:1) to give the title compound (2 g, 82.0% yield) as a white solid.

LCMS: m/z 473.2 (M+H)⁺ (ES⁺).

¹H NMR (CDCl₃): δ 7.73 (s, 1H), 7.58 (s, 1H), 7.15 (d, 4H), 6.83 (d, 4H), 6.03 (br s, 1H), 5.36 (br s, 1H), 4.24 (s, 4H), 3.80 (s, 6H), 1.79 (s, 6H).

### Step B: 1-(1-amino-2-methylpropan-2-yl)-N,N-bis(4-methoxybenzyl)-1H-pyrazole-4-sulfonamide

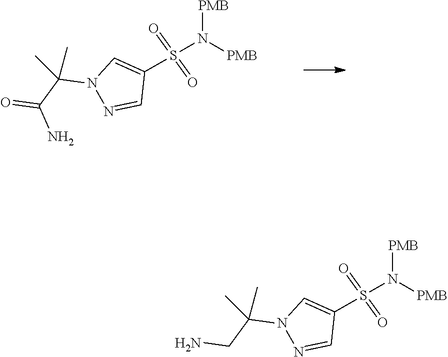

To a solution of 2-(4-(N,N-bis(4-methoxybenzyl)sulfamoyl)-1H-pyrazol-1-yl)-2-methyl-propanamide (2 g, 4.23 mmol) in THF (20 mL) was added BH₃-Me₂S (10 M, 1.27 mL) at 0° C. in portions under N₂. The resultant mixture was stirred at 60° C. for 12 h, then quenched with MeOH (30 mL) and then 1 M aq HCl (10 mL) at 0° C. The mixture was stirred at 25° C. for 30 min, then concencentrated in vacuum. The residue was purified by reverse phase flash chromatography (water (0.1% TFA)-MeCN) to give the title compound (1.21 g, 57.8% yield, HCl salt) as a white solid.

LCMS: m/z 459.1 (M−HCl+H)⁺ (ES⁺).

¹H NMR (DMSO-d₆): δ 8.33 (s, 1H), 8.13 (br s, 3H), 7.91 (s, 1H), 7.09 (d, 4H), 6.82 (d, 4H), 4.18 (s, 4H), 3.72 (s, 6H), 2.51 (s, 2H), 1.58 (s, 6H).

### Intermediate B1: 2-(2-(2-fluoropyridin-4-yl)-6-isopropylphenyl)acetic acid

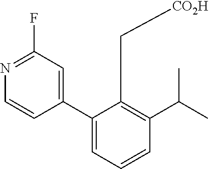 [SMILES: CC(C)c1cccc(-c2ccnc(F)c2)c1CC(=O)O]

### Step A: methyl 2-(2-chloro-6-(2-fluoropyridin-4-yl)phenyl)acetate

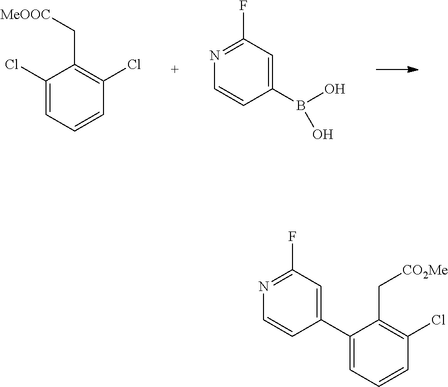

A solution of K₂CO₃ (44.9 g, 325 mmol) in water (50 mL) was added to methyl 2-(2,6-dichlorophenyl)acetate (17.8 g, 81 mmol), (2-fluoropyridin-4-yl)boronic acid (11.45 g, 81 mmol), XPhos (3.87 g, 8.13 mmol) and Pd₂(dba)₃ (3.72 g, 4.06 mmol) in dioxane (500 mL) and the suspension was evacuated and backfilled with N₂ three times whilst stirring at 60° C., then the reaction was stirred at 90° C. for 1 h. The mixture was diluted with EtOAc (200 mL) and washed with water (200 mL) and brine (200 mL). The organic layer was separated, dried (MgSO₄) and the product was purified by FC (0-20% EtOAc/isohexane) to afford the title compound (11.5 g, 47%) as a white solid.

LCMS m/z 280.1/282.2 (M+H)⁺ (ES⁺).

¹H NMR (CDCl₃) δ 8.33 (d, J=5.1 Hz, 1H), 7.61 (dd, J=8.1, 1.3 Hz, 1H), 7.46 (t, J=7.9 Hz, 1H), 7.33-7.30 (m, 1H), 7.30-7.26 (m, 1H), 7.14 (br s, 1H), 3.72 (s, 2H), 3.59 (s, 3H).

### Step B: methyl 2-(2-(2-fluoropyridin-4-yl)-6-(prop-1-en-2-yl)phenyl)acetate

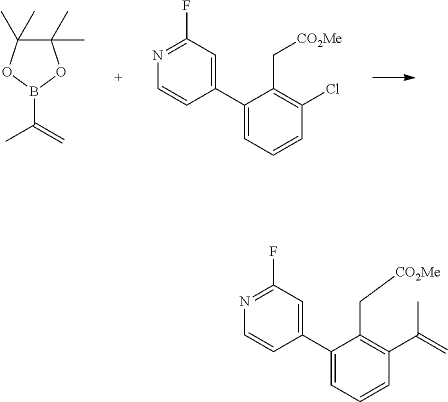

Dioxane (200 mL) was added to methyl 2-(2-chloro-6-(2-fluoropyridin-4-yl)phenyl)-acetate (11.5 g, 41.1 mmol), Pd₂dba₃ (1.05 g, 1.240 mmol) and XPhos (1.2 g, 2.52 mmol), under N₂, followed by 4,4,5,5-tetramethyl-2-(prop-1-en-2-yl)-1,3,2-dioxaborolane (7.75 ml, 41.2 mmol) and a solution of K₂CO₃ (17.05 g, 123 mmol) in water (20 mL). The reaction was heated at 95° C. for 20 h. After cooling to RT, the mixture was diluted with EtOAc (100 mL) and washed with 3:1 water/brine (2×200 mL). The organic layer was separated, dried (MgSO₄) and concentrated in vacuo. The crude product was purified by FC (0-30% EtOAc/isohexane) to afford the title compound (10.77 g, 90%) as a pale yellow oil.

LCMS m/z 286.1 (M+H)⁺ (ES⁺).

¹H NMR (DMSO-d₆) δ 8.29 (d, J=5.1 Hz, 1H), 7.39 (t, J=7.6 Hz, 1H), 7.29-7.25 (m, 2H), 7.20 (dd, J=7.6, 1.4 Hz, 1H), 7.14-7.10 (m, 1H), 5.31-5.18 (m, 1H), 4.90-4.76 (m, 1H), 3.64 (br s, 2H), 3.46 (br s, 3H), 2.00 (br s, 3H).

### Step C: methyl 2-(2-(2-fluoropyridin-4-yl)-6-isopropylphenyl)acetate

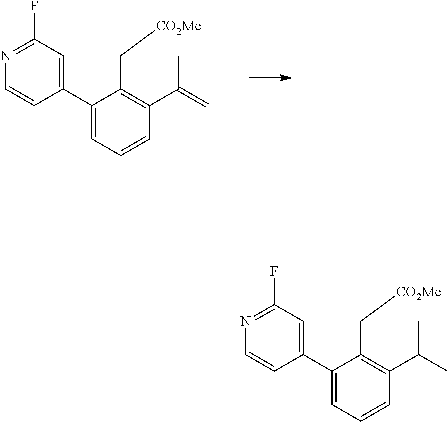 [SMILES: C=C(C)c1cccc(-c2ccnc(F)c2)c1CC(=O)OC ; COC(=O)Cc1c(-c2ccnc(F)c2)cccc1C(C)C]

Methyl 2-(2-(2-fluoropyridin-4-yl)-6-(prop-1-en-2-yl)phenyl)acetate (275 mg, 0.964 mmol) and 10% Pd/C (103 mg, 0.096 mmol) were suspended in EtOH (20 mL). The reaction was stirred at RT under 2 atm H₂ for 18 h. The reaction mixture was filtered through a glass fibre filter, washing with MeOH, and concentrated in vacuo to afford the title compound (290 mg, 99%) as a pale yellow oil.

LCMS m/z 288.0 (M+H)⁺ (ES⁺).

¹H NMR (CDCl₃) δ 8.23 (d, J=5.2 Hz, 1H), 7.41 (dd, J=7.9, 1.5 Hz, 1H), 7.35 (t, J=7.7 Hz, 1H), 7.17-7.14 (m, 1H), 7.04 (dd, J=7.4, 1.5 Hz, 1H), 6.91 (t, J=1.6 Hz, 1H), 3.68 (s, 3H), 3.59 (s, 2H), 3.06 (sept, J=6.8 Hz, 1H), 1.25 (d, J=6.8 Hz, 6H).

### Step D: 2-(2-(2-fluoropyridin-4-yl)-6-isopropylphenyl)acetic acid

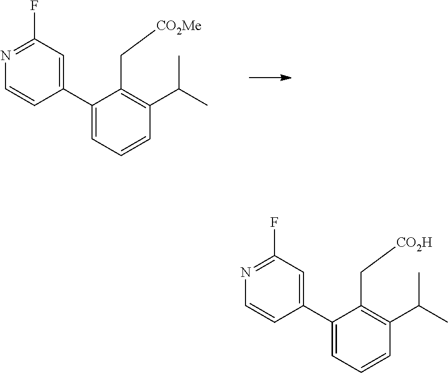 [SMILES: COC(=O)Cc1c(-c2ccnc(F)c2)cccc1C(C)C ; CC(C)c1cccc(-c2ccnc(F)c2)c1CC(=O)O]

2 M NaOH (486 μL, 0.972 mmol) was added to a solution of methyl 2-(2-(2-fluoro-pyridin-4-yl)-6-isopropylphenyl)acetate (254 mg, 0.884 mmol) in THF (5 mL) and the reaction stirred at RT for 3 h. Additional 2 M NaOH (486 μL, 0.972 mmol) was added and the reaction heated at 60° C. for 16 h. Further 2 M NaOH (972 μL, 1.944 mmol) was added and the reaction heated at 60° C. for 4 days. The reaction mixture was diluted with EtOAc (20 mL) and acidified to pH <4 using aq 1 M HCl. The layers were separated and the aqueous layer extracted with EtOAc (2×10 mL). The combined organic layers were dried (phase separator) and concentrated in vacuo to afford the title compound (0.210 g, 82%) as an orange oil.

LCMS m/z 274.2 (M+H)⁺ (ES⁺).

¹H NMR (DMSO-d₆) δ 12.38 (s, 1H), 8.30 (d, J=5.1 Hz, 1H), 7.48-7.41 (m, 1H), 7.37 (t, J=7.7 Hz, 1H), 7.29-7.25 (m, 1H), 7.12-7.05 (m, 2H), 3.53 (s, 2H), 3.07 (sept, J=6.8 Hz, 1H), 1.20 (d, J=6.7 Hz, 6H).

### Intermediate B2: tert-butyl 2-(2-(2-fluoropyridin-4-yl)-6-isopropyl-3-methyl-phenyl)acetate

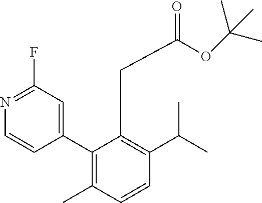 [SMILES: Cc1ccc(C(C)C)c(CC(=O)OC(C)(C)C)c1-c1ccnc(F)c1]

### Step A: 2-(2-fluoropyridin-4-yl)-6-isopropyl-3-methylphenol

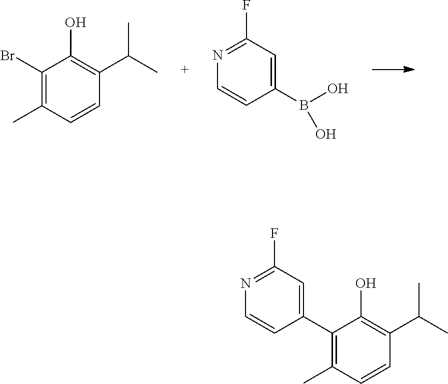

N₂ was bubbled through a stirred mixture of 2-bromo-6-isopropyl-3-methylphenol (2 g, 8.73 mmol), (2-fluoropyridin-4-yl)boronic acid (1.2 g, 8.52 mmol) and K₂CO₃ (3.62 g, 26.2 mmol) in dioxane (30 mL) and water (5 mL) for 5 min. PdCl₂(dppf). CH₂Cl₂ (319 mg, 0.436 mmol) was added and the mixture heated at 80° C. for 20 h. The mixture was cooled to RT, then partitioned between EtOAc (100 mL) and water (50 mL). The organic layer was dried (MgSO₄) and evaporated and the residue was purified by FC (0-50% EtOAc/isohexane) to afford the title compound (1.26 g, 58% yield) as a white solid.

LCMS m/z 246.1 (M+H)⁺ (ES⁺).

¹H NMR (DMSO-d₆) δ 8.27 (d, J=5.0 Hz, 1H), 8.08 (s, 1H), 7.21 (dt, J=5.2, 1.6 Hz, 1H), 7.12 (d, J=7.8 Hz, 1H), 7.06 (s, 1H), 6.81 (d, J=7.9 Hz, 1H), 3.29-3.21 (m, 1H), 1.97 (s, 3H), 1.16 (d, J=6.8 Hz, 6H).

### Step B: 2-(2-fluoropyridin-4-yl)-6-isopropyl-3-methylphenyl trifluoromethane-sulfonate

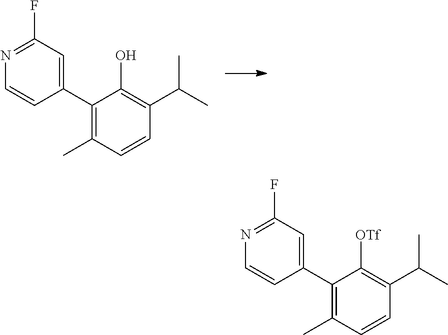 [SMILES: Cc1ccc(C(C)C)c(O)c1-c1ccnc(F)c1 ; Cc1ccc(C(C)C)c(OS(=O)(=O)C(F)(F)F)c1-c1ccnc(F)c1]

A solution of 2-(2-fluoropyridin-4-yl)-6-isopropyl-3-methylphenol (1.27 g, 5.18 mmol) in DCM (20 mL) was cooled to 0° C. Pyridine (0.63 mL, 7.76 mmol) and Tf₂O (7.76 mL, 7.76 mmol) were added sequentially to the stirred solution and the reaction mixture was warmed to RT and stirred for 18 h. The reaction was then diluted with DCM (50 mL) and washed with water (50 mL) and brine (50 mL). The organic layer was dried (phase separator) and concentrated in vacuo to afford the title compound (1.72 g, 87%) as a light brown solid.

¹H NMR (DMSO-d₆) δ 8.37 (d, J=5.1 Hz, 1H), 7.61 (d, J=8.1 Hz, 1H), 6.49 (d, J=8.1 Hz, 1H), 7.28-735 (m, 1H), 7.29 (s, 1H), 3.22 (sept, J=6.8 Hz, 1H), 2.12 (s, 3H), 1.26 (d, J−6.8 Hz, 6H).

### Step C: tert-butyl 2-(2-(2-fluoropyridin-4-yl)-6-isopropyl-3-methylphenyl)acetate

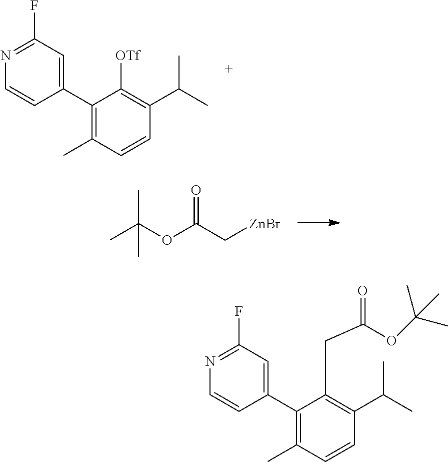

(2-(tert-Butoxy)-2-oxoethyl)zinc(II) bromide (0.33 M in THF, 6.02 mL, 1.987 mmol) was added to a solution of 2-(2-fluoropyridin-4-yl)-6-isopropyl-3-methylphenyl trifluoromethanesulfonate (300 mg, 0.795 mmol), tetrabutylammonium bromide (384 mg, 1.192 mmol) and Xantphos-Pd-G3 (151 mg, 0.159 mmol) in THF (2 mL). The reaction was degassed with N₂ and stirred at 70° C. for 72 h under N₂. The reaction was cooled to RT, quenched with water (15 mL) and extracted with EtOAc (2×15 mL). The combined organic layers were dried (phase separator) and concentrated in vacuo. The crude product was purified by FC (0-50% EtOAc/isohexane) to afford the title compound (215 mg, 78%) as an off-white solid.

LCMS m/z 344.3 (M+H)⁺ (ES⁺).

¹H NMR (DMSO-d₆) δ 8.33 (d, J=5.0 Hz, 1H), 7.32 (d, J=8.0 Hz, 1H), 7.23 (d, J=8.1 Hz, 1H), 7.16-7.11 (m, 1H), 6.95-6.92 (m, 1H), 3.42-3.31 (m, 2H), 3.03 (sept, J=6.8 Hz, 1H), 1.93 (s, 3H), 1.32 (s, 9H), 1.20-1.16 (m, 6H).

### Intermediate B3: tert-butyl 2-(5-(2-fluoropyridin-4-yl)-2,3-dihydro-1H-inden-4-yl)-acetate

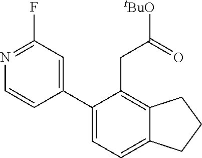 [SMILES: CC(C)(C)OC(=O)Cc1c(-c2ccnc(F)c2)ccc2c1CCC2]

### Step A: 5-(2-fluoropyridin-4-yl)-2,3-dihydro-1H-inden-4-amine

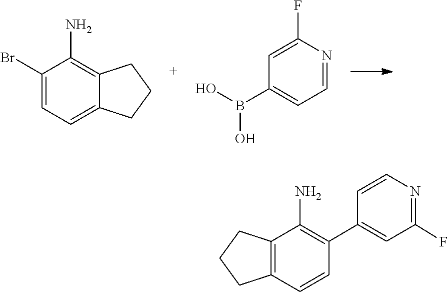 [SMILES: Nc1c(Br)ccc2c1CCC2 ; OB(O)c1ccnc(F)c1 ; Nc1c(-c2ccnc(F)c2)ccc2c1CCC2]

A mixture of 5-bromo-2,3-dihydro-1H-inden-4-amine (10 g, 47.2 mmol), (2-fluoro-pyridin-4-yl)boronic acid (6.64 g, 47.2 mmol) and K₂CO₃ (19.6 g, 142 mmol) in dioxane (200 mL) and water (50 mL) was degassed with N₂. PdCl₂(dppf) (1.7 g, 2.323 mmol) was added and the reaction heated at 80° C. for 20 h. After cooling at RT, the reaction was partitioned between EtOAc (100 mL) and water (50 mL). The organic layer was dried (MgSO₄) and evaporated in vacuo. The residue was purified by FC (0-50% EtOAc/isohexane) to afford the title compound (8.64 g, 79%) as a white solid.

LCMS m/z 229.1 (M+H)⁺ (ES⁺).

¹H NMR (DMSO-d₆) δ 8.24 (d, J=5.2 Hz, 1H), 7.38 (ddd, J=5.2, 2.2, 1.4 Hz, 1H), 7.16 (d, J=1.4 Hz, 1H), 6.90 (d, J=7.6 Hz, 1H), 6.60 (d, J=7.6 Hz, 1H), 4.82 (s, 2H), 2.84 (t, J=7.5 Hz, 2H), 2.71 (t, J=7.4 Hz, 2H), 2.03 (p, J=7.5 Hz, 2H).

### Step B: 5-(2-fluoropyridin-4-yl)-2,3-dihydro-1H-inden-4-ol

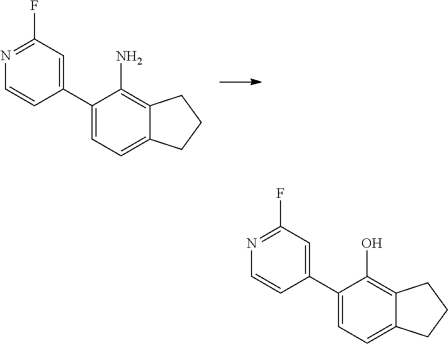 [SMILES: Nc1c(-c2ccnc(F)c2)ccc2c1CCC2 ; Oc1c(-c2ccnc(F)c2)ccc2c1CCC2]

Sodium nitrite (1.04 g, 15.07 mmol) in water (10 mL) was added to a solution of 5-(2-fluoropyridin-4-yl)-2,3-dihydro-1H-inden-4-amine (2.65 g, 11.61 mmol) in H₂SO₄ (2 M in THF, 50 mL) at 0° C. and the reaction was stirred for 40 min. This solution was added slowly to H₂SO₄ (2 M in THF, 50 mL) and the reaction was stirred at 50° C. for 40 min, diluted with water (100 mL) and extracted with EtOAc (150 mL). The aqueous phase was basified to pH ˜8 with 2 M NaOH and extracted with EtOAc (150 mL). The combined organic phases were dried (MgSO₄) and concentrated in vacuo. The crude product was purified by FC (0-60% EtOAc/isohexane) to afford the title compound (1.71 g, 59%) as a yellow solid.

LCMS m/z 230.1 (M+H)⁺ (ES⁺).

¹H NMR (DMSO-d₆) δ 9.08 (br s, 1H), 8.21 (d, J=5.3 Hz, 1H), 7.54-7.49 (m, 1H), 7.30 (s, 1H), 7.19 (d, J=7.7 Hz, 1H), 6.85 (d, J=7.7 Hz, 1H), 2.90-2.84 (m, 4H), 2.03 (p, J=7.5 Hz, 2H).

### Step C: 5-(2-fluoropyridin-4-yl)-2,3-dihydro-1H-inden-4-yl trifluoromethanesulfonate

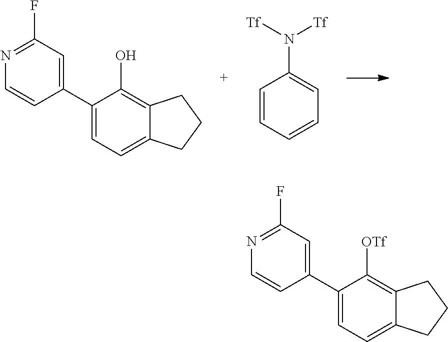

1,1,1-Trifluoro-N-phenyl-N-((trifluoromethyl)sulfonyl)methanesulfonamide (3.20 g, 8.95 mmol) was added portion-wise to a solution of 5-(2-fluoropyridin-4-yl)-2,3-dihydro-1H-inden-4-ol (1.71 g, 7.46 mmol) and DIPEA (3.26 mL, 18.65 mmol) in DCM (25 mL). The reaction was stirred for 2 days, then diluted with DCM (35 ml), washed with aq 1 M HCl (2×100 mL) and sat aq NaHCO₃ (100 mL). The organic phase was dried (MgSO₄) and concentrated in vacuo. The crude product was purified by FC (0-50% EtOAc/isohexane) then taken up in DCM (100 mL) and washed with sat aq NaHCO₃ (100 mL). The organic phase was dried (MgSO₄) and concentrated in vacuo to afford the title compound (2.18 g, 81%).

LCMS m/z 362.0 (M+H)⁺ (ES⁺).

¹H NMR (DMSO-d₆) δ 8.34 (d, J=5.2 Hz, 1H), 7.50 (d, J=7.7 Hz, 1H), 7.47 (dt, J=5.2, 1.7 Hz, 1H), 7.45 (d, J=7.6 Hz, 1H), 7.35 (br s, 1H), 3.07-3.01 (m, 4H), 2.15 (p, J=7.5 Hz, 2H).

### Step D: tert-butyl 2-(5-(2-fluoropyridin-4-yl)-2,3-dihydro-1H-inden-4-yl)acetate

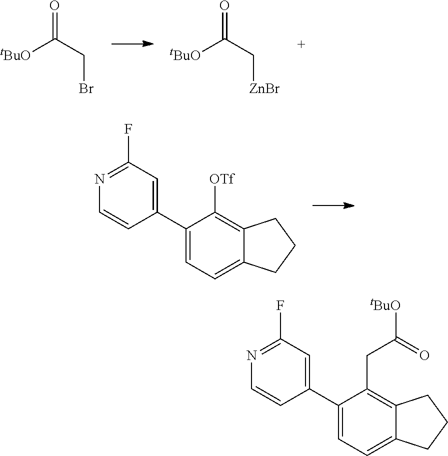

Zn dust (12.97 g, 198 mmol) was suspended in 1 M HCl (100 mL) and stirred at RT for 1 h under N₂. The supernatant was removed with a syringe and the zinc washed with EtOH (2×100 mL) and THF (2×100 mL). The Zn dust was dried under vacuum for 16 h and taken up in THF (150 mL). TMSCl (1.70 mL, 13.30 mmol) and 1,2-dibromoethane (1.14 mL, 13.23 mmol) were added and the mixture was heated to reflux for 1 h. tert-Butyl 2-bromoacetate (15 mL, 66.1 mmol) was added dropwise whilst maintaining controlled reflux and the reaction was heated to reflux for 1 h. The supernatant containing the crude (2-(tert-butoxy)-2-oxoethyl)zinc(II) bromide (34.3 mL, 15.08 mmol) was added to a solution of 5-(2-fluoropyridin-4-yl)-2,3-dihydro-1H-inden-4-yl trifluoromethanesulfonate (2.18 g, 6.03 mmol), Pd₂(dba)₃ (0.55 g, 0.601 mmol) and XPhos (0.58 g, 1.217 mmol) in THF (10 mL) and the solution was stirred at 70° C. for 1 h. After cooling to RT, the reaction was diluted with EtOAc (100 mL) and water (200 mL) and the residue was filtered. The phases were separated. The aqueous phase was extracted with EtOAc (2×150 mL), the combined organics were dried (MgSO₄), loaded onto silica and purified by FC (0-50% EtOAc/isohexane) followed by FC (0-10% MTBE/isohexane) to afford the title compound (1.64 g, 75%) as a colourless oil.

LCMS m/z 328.1 (M+H)⁺ (ES⁺).

¹H NMR (DMSO-d₆) δ 8.29 (d, J=5.1 Hz, 1H), 7.28-7.24 (m, 2H), 7.10-7.05 (m, 2H), 3.51 (s, 2H), 2.95 (t, J=7.5 Hz, 2H), 2.86 (t, J=7.4 Hz, 2H), 2.06 (p, J=7.5 Hz, 2H), 1.33 (s, 9H).

### Intermediate B4: tert-butyl 2-(4-fluoro-2-(2-fluoropyridin-4-yl)-6-isopropylphenyl)-acetate

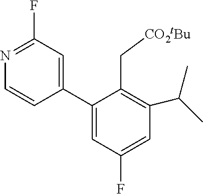 [SMILES: CC(C)c1cc(F)cc(-c2ccnc(F)c2)c1CC(=O)OC(C)(C)C]

### Step A: 2-bromo-4-fluoro-6-isopropylaniline

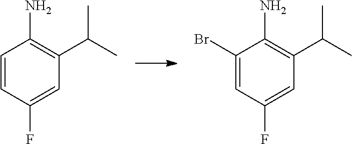

NBS (7.16 g, 40.2 mmol) was added portion wise to a solution of 4-fluoro-2-isopropyl-aniline (6.16 g, 40.2 mmol) in DCM (150 mL) at 0° C. The reaction was stirred at 0° C. for 1.5 h. The reaction was diluted with DCM (100 mL), washed with water (200 mL) and the organic phase was further washed with sat aq Na₂S₂O₃ (200 mL). The organic phase was separated, dried (MgSO₄), filtered and purified by FC (0-30% EtOAc/isohexane) to afford the title compound (6.62 g, 69%) as a deep purple oil.

LCMS m/z 232.1, 234.0 (M+H)⁺ (ES⁺).

¹H NMR (CDCl₃) δ 7.06 (dd, J=7.7, 2.9 Hz, 1H), 6.85 (dd, J=9.8, 2.9 Hz, 1H), 3.98 (br s, 2H), 2.95-2.85 (m, 1H), 1.25 (d, J=6.8 Hz, 6H).

### Step B: 4-fluoro-2-(2-fluoropyridin-4-yl)-6-isopropylaniline

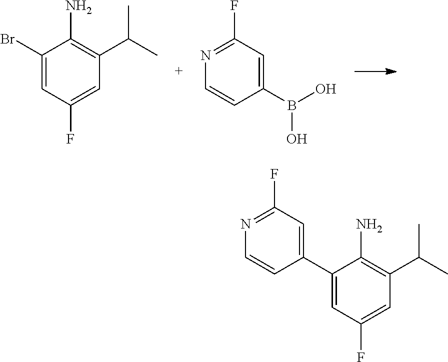

A solution of 2-bromo-4-fluoro-6-isopropylaniline (5.60 g, 24.13 mmol) in dry 1,4-dioxane (200 mL) was added to a mixture of (2-fluoropyridin-4-yl)boronic acid (3.40 g, 24.13 mmol) and Pd(dppf)Cl₂.DCM (1.0 g, 1.225 mmol) followed by a solution of potassium carbonate (13.30 g, 96 mmol) in water (20 mL). The resulting suspension was evacuated and backfilled with N₂ twice before stirring at 95° C. for 1 h. The reaction was diluted with EtOAc (200 mL) and washed with water (250 mL). The organic phase was separated, dried (MgSO₄), filtered and directly loaded onto silica for purification. The crude product was purified by FC (0-50% EtOAc/isohexane) to afford the title compound (4.93 g, 79%) as a purple oil.

LCMS m/z 249.3 (M+H)⁺ (ES⁺).

¹H NMR (DMSO-d₆) δ 8.28 (d, J=5.1 Hz, 1H), 7.43-7.39 (m, 1H), 7.23-7.20 (m, 1H), 6.97 (dd, J=10.2, 3.0 Hz, 1H), 6.79 (dd, J=9.0, 3.0 Hz, 1H), 4.63 (br s, 2H), 3.13-3.03 (m, 1H), 1.18 (d, J=6.7 Hz, 6H).

### Step C: 4-(2-bromo-5-fluoro-3-isopropylphenyl)-2-fluoropyridine

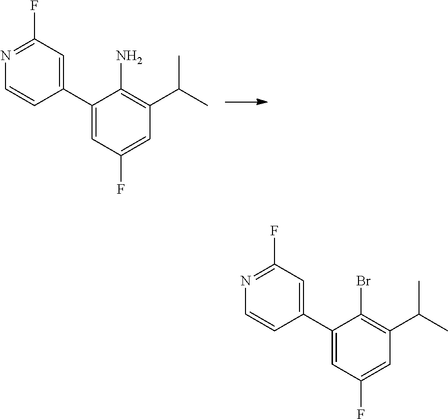 [SMILES: CC(C)c1cc(F)cc(-c2ccnc(F)c2)c1N ; CC(C)c1cc(F)cc(-c2ccnc(F)c2)c1Br]

To a mixture of 4-fluoro-2-(2-fluoropyridin-4-yl)-6-isopropylaniline (6.5 g, 24.87 mmol), CuBr (4.91 g, 34.2 mmol) and CuBr₂ (0.032 g, 0.143 mmol) was added MeCN (75 mL). The suspension was cooled to 0° C. over 10 min. Neat isopentyl nitrite (4.6 mL, 34.2 mmol) was added to the solution dropwise and then the reaction mixture was left to stir at 50° C. for 2 h. The reaction mixture was left to cool to RT, filtered through a pad of Celite® and concentrated in vacuo. The crude product was purified by FC (0-15% EtOAc/isohexane) to afford the title compound (4.98 g, 53%) as a pink solid.

LCMS m/z 312.1, 314.2 (M+H)⁺ (ES⁺).

¹H NMR (DMSO-d₆) δ 8.34 (d, J=5.1 Hz, 1H), 7.44-7.36 (m, 2H), 7.28 (s, 1H), 7.24 (dd, J=8.5, 3.0 Hz, 1H), 3.40 (p, J=6.8 Hz, 1H), 1.25 (d, J=6.8 Hz, 6H).

### Step D: tert-butyl 2-(4-fluoro-2-(2-fluoropyridin-4-yl)-6-isopropylphenyl)acetate

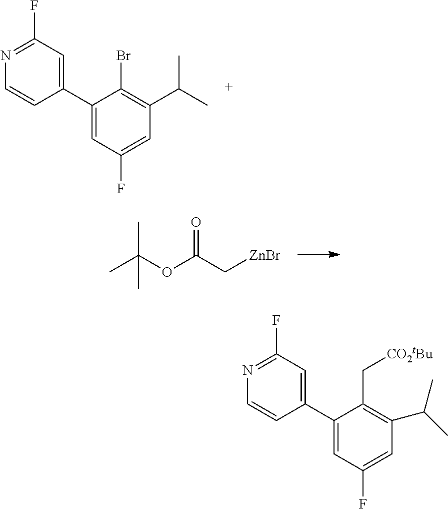

4-(2-Bromo-5-fluoro-3-isopropylphenyl)-2-fluoropyridine (2.46 g, 7.88 mmol) and XPhos (0.752 g, 1.577 mmol) were suspended in dry THF (5 mL). The mixture was degassed (N₂) for 5 min, then evacuated and back-filled with N₂ (×3), then Pd₂(dba)₃ (0.722 g, 0.789 mmol) was added and the reaction evacuated and back-filled with N₂ (×3). (2-(tert-Butoxy)-2-oxoethyl)zinc(II) bromide (0.41 M in THF) (57-7 mL, 23.66 mmol) was added and the reaction stirred at reflux for 3 h. The reaction was diluted with MTBE (200 mL) and filtered. The filtrate was washed with brine (200 mL), dried using a phase separator and concentrated in vacuo. The crude product was purified by FC (0-30% EtOAc/isohexane) to afford the title compound (2.44 g, 80%) as a yellow oil.

LCMS m/z 348.5 (M+H)⁺ (ES⁺).

¹H NMR (DMSO-d₆) δ 8.33 (d, J=5.9 Hz, 1H), 7.32-7.26 (m, 2H), 7.11-7.09 (m, 1H), 7.00 (dd, J=8.8, 2.8 Hz, 1H), 2.50 (s, 2H), 3.12-3.05 (m, 1H), 1.34 (s, 9H), 1.20 (d, J=6.8 Hz, 6H).

### Intermediate B5: methyl 2-(4-fluoro-2-isopropyl-6-(2-oxo-1,2-dihydropyridin-4-yl)-phenyl)acetate

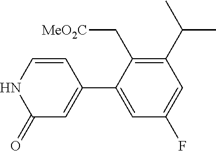 [SMILES: COC(=O)Cc1c(-c2cc[nH]c(=O)c2)cc(F)cc1C(C)C]

### Step A: 2-(4-fluoro-2-isopropyl-6-(2-oxo-1,2-dihydropyridin-4-yl)phenyl)acetic acid

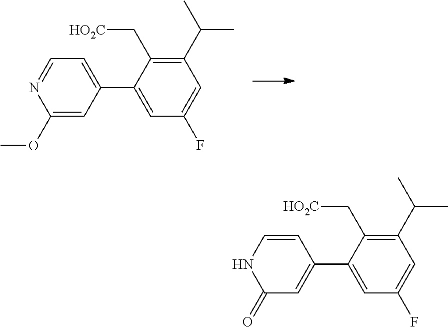 [SMILES: COc1cc(-c2cc(F)cc(C(C)C)c2CC(=O)O)ccn1 ; CC(C)c1cc(F)cc(-c2cc[nH]c(=O)c2)c1CC(=O)O]

A mixture of 2-(4-fluoro-2-isopropyl-6-(2-methoxypyridin-4-yl)phenyl)acetic acid (2.2 g, 7.25 mmol) in dioxane and aq 1 M HCl (15 mL) was heated at 70° C. for 5 h, conc HCl (0.5 mL) added and heated for a further 48 h. A further portion of conc HCl (1 mL) was added, and the mixture was heated at 70° C. for 48 h. The mixture was cooled, filtered and washed with water (5 mL). The solid was evaporated from MeOH (2×20 mL) and dried to afford the title compound (1.00 g, 46%) as an off-white solid.

LCMS m/z 288.2 (M−H)⁻ (ES⁻).

¹H NMR (DMSO-d₆) δ 12.40 (br s, 1H), 11.76 (br s, 1H), 7.43 (d, J=6.6 Hz, 1H), 7.22 (dd, J=10.5, 2.8 Hz, 1H), 6.90 (dd, J=8.9, 2.8 Hz, 1H), 6.17 (d, J=1.7 Hz, 1H), 6.07 (dd, J=6.6, 1.7 Hz, 1H), 3.54 (s, 2H), 3.07-2.96 (m, 1H), 1.18 (d, J=6.7 Hz, 6H).

### Step B: methyl 2-(4-fluoro-2-isopropyl-6-(2-oxo-1,2-dihydropyridin-4-yl)phenyl)-acetate

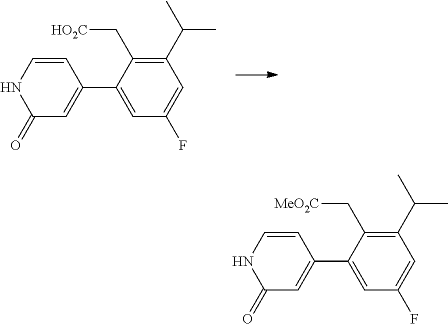 [SMILES: CC(C)c1cc(F)cc(-c2cc[nH]c(=O)c2)c1CC(=O)O ; COC(=O)Cc1c(-c2cc[nH]c(=O)c2)cc(F)cc1C(C)C]

AcCl (3 mL, 42.2 mmol) was added dropwise to MeOH (20 mL) cooled with an ice bath. The mixture was allowed to warm to RT, then 2-(4-fluoro-2-isopropyl-6-(2-oxo-1,2-dihydropyridin-4-yl)phenyl)acetic acid (990 mg, 3.42 mmol) was added and the mixture stirred for 24 h. The solvent was evaporated, and the residue purified by FC (0-10% MeOH/DCM) to afford the title compound (1.04 g, 98%) as a white powder.

LCMS m/z 303.8 (M+H)⁺ (ES⁺).

¹H NMR (CDCl₃) δ 7.86-7.78 (br m, 1H), 7.12 (dd, J=10.1, 2.6 Hz, 1H), 6.88-6.76 (m, 2H), 6.72-6.64 (br m, 1H), 3.70 (s, 3H), 3.60 (s, 2H), 3.10-3.01 (m, 1H), 1.25 (d, J=6.8 Hz, 6H). One exchangeable proton not observed.

### Intermediate B6: 2-(2-cyclopropyl-6-(2-fluoropyridin-4-yl)phenyl)acetic acid

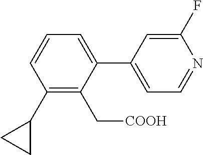 [SMILES: O=C(O)Cc1c(-c2ccnc(F)c2)cccc1C1CC1]

### Step A: methyl 2-(2-chloro-6-(2-fluoropyridin-4-yl)phenyl)acetate

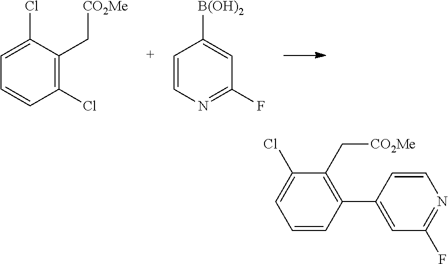

A solution of potassium carbonate (33.0 g, 240 mmol) in water (36 mL) was added to a solution of methyl 2-(2,6-dichlorophenyl)acetate (13.0 g, 59 mmol), (2-fluoropyridin-4-yl)boronic acid (8.40 g, 59 mmol), XPhos (2.80 g, 5.9 mmol) and Pd₂(dba)₃ (2.70 g, 2.95 mmol) in anhydrous 1,4-dioxane (365 mL) and the suspension was evacuated and backfilled with N₂ three times whilst stirring at 60° C., then the reaction was stirred at 90° C. for 1.5 h. The solvent was removed and the resultant liquor was diluted with EtOAc (200 mL) and washed with water (200 mL) and brine (200 mL). The organic phase was separated, dried (MgSO₄) and filtered. The crude product was purified by FC (0-20% EtOAc/isohexane) to afford methyl 2-(2-chloro-6-(2-fluoropyridin-4-yl)-phenyl)acetate (4.8 g, 17 mmol, 28%) as a pale yellow solid. Fractions containing methyl 2-(2,6-bis(2-fluoropyridin-4-yl)phenyl)acetate and methyl 2-(2-chloro-6-(2-fluoropyridin-4-yl)phenyl)acetate were re-purified by FC (0-20% EtOAc/isohexane) to afford methyl 2-(2-chloro-6-(2-fluoropyridin-4-yl)phenyl)acetate as an off-white solid (1.63 g). In total 6.43 g (39%) of title compound were obtained.

¹H NMR (CDCl₃) δ 8.27 (d, J=5.0 Hz, 1H), 7.50 (dd, J=8.1, 1.3 Hz, 1H), 7.33 (t, J=7.9 Hz, 1H), 7.16 (m, 2H), 6.93-6.89 (m, 1H), 3.72 (s, 3H), 3.68 (s, 2H).

### Step B: methyl 2-(2-cyclopropyl-6-(2-fluoropyridin-4-yl)phenyl)acetate

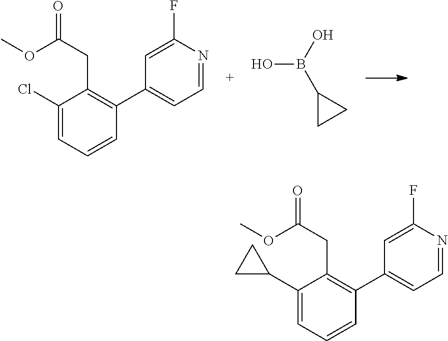 [SMILES: COC(=O)Cc1c(Cl)cccc1-c1ccnc(F)c1 ; OB(O)C1CC1 ; COC(=O)Cc1c(-c2ccnc(F)c2)cccc1C1CC1]

A suspension of methyl 2-(2-chloro-6-(2-fluoropyridin-4-yl)phenyl)acetate (2.00 g, 7.15 mmol), potassium phosphate (5.46 g, 25.7 mmol), cyclopropylboronic acid (921 mg, 10.7 mmol) and tricyclohexylphosphane (100 mg, 358 μmol) in toluene (50 mL) and water (8 mL) was degassed with N₂ (5 min), then evacuated and back-filled with N₂ (3×). Palladium (II) acetate (80.3 mg, 358 μmol) was added and the reaction mixture was refluxed for 18 h. The reaction was allowed to cool to RT before dilution with EtOAc (200 mL), washed with water (200 mL) then brine (100 mL) followed by removal of the solvent under reduced pressure. The crude product was purified by FC (0-50% EtOAc/isohexane) to afford the title compound (0.56 g, 23%) as a pale yellow oil.

¹H NMR (CDCl₃) δ 8.24 (d, J=5.1, 1H), 7.29 (t, J=7.7 Hz, 1H), 7.19-7.14 (m, 2H), 7.07 (dd, J=7.6, 1.4 Hz, 1H), 6.94-6.92 (m, 1H), 3.75 (s, 2H), 3.68 (s, 3H), 1.86 (m, J=8.5, 5.4 Hz, 1H), 0.99-0.93 (m, 2H), 0.72-0.66 (m, 2H).

### Step C: 2-(2-cyclopropyl-6-(2-fluoropyridin-4-yl)phenyl)acetic acid

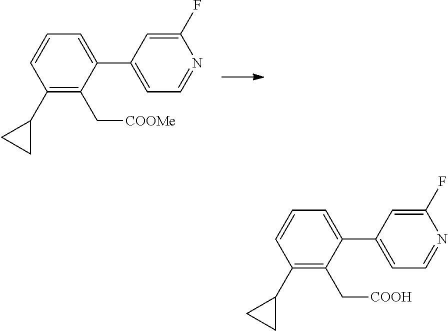 [SMILES: COC(=O)Cc1c(-c2ccnc(F)c2)cccc1C1CC1 ; O=C(O)Cc1c(-c2ccnc(F)c2)cccc1C1CC1]

To a stirred solution of methyl 2-(2-cyclopropyl-6-(2-fluoropyridin-4-yl)phenyl)acetate (580 mg, 2.03 mmol) in dry 1,4-dioxane (10 mL) was added 2 M aq NaOH (2 mL) The reaction mixture was stirred for 3.5 h at 90° C. The solution was acidified to ˜pH 2 then diluted with EtOAc (25 mL) and transferred into a separatory funnel. The aqueous layer was extracted with EtOAc (2×10 mL). The combined organic layers were washed with brine (25 mL, dried over MgSO₄, filtered and concentrated in vacuo. The crude product was purified by FC (0-70% EtOAc/isohexane) to afford the title compound (440 mg, 64%) as a pale yellow oil.

¹H NMR (CDCl₃) δ 8.26 (d, J=5.1 Hz, 1H), 7.31 (t, J=7.7 Hz, 1H), 7.21-7.14 (m, 2H), 7.08 (dd, J=7.6, 1.4 Hz, 1H), 6.93 (d, J=1.8 Hz, 1H), 3.81 (s, 2H), 1.90 (ddd, J=13.9, 8.5, 5.5 Hz, 1H), 1.03-0.98 (m, 2H), 0.74-0.70 (m, 2H). One exchangeable proton not observed.

### Intermediate B7: tert-butyl 2-(3,4-difluoro-2-(2-fluoropyridin-4-yl)-6-isopropyl-phenyl)acetate

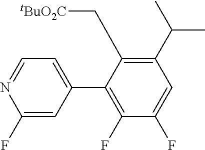 [SMILES: CC(C)c1cc(F)c(F)c(-c2ccnc(F)c2)c1CC(=O)OC(C)(C)C]

### Step A: 4,5-difluoro-2-(prop-1-en-2-yl)aniline

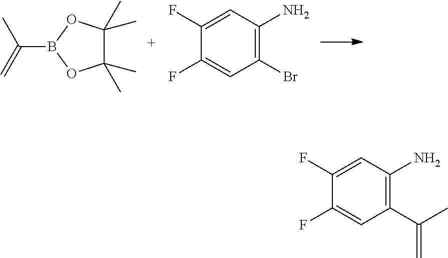

To a solution of 2-bromo-4,5-difluoroaniline (10 g, 48 mmol), 4,4,5,5-tetramethyl-2-(prop-1-en-2-yl)-1,3,2-dioxaborolane (8.1 g, 48 mmol) and PdCl₂(dppf). DCM (2.0 g, 2.4 mmol) in anhydrous 1,4-dioxane (200 mL) was added a solution of K₂CO₃ (20 g, 0.14 mol) in water (20 mL) and the resulting suspension was evacuated and backfilled with N₂ twice before stirring at 100° C. for 3 h. The reaction mixture was partitioned between EtOAc (20 mL) and water (20 mL). The organic phase was separated, the aqueous was further extracted with EtOAc (20 mL), the organic phases were combined, dried (MgSO₄), filtered and concentrated under reduced pressure. The crude product was purified by FC (0-30% EtOAc/isohexane) to afford the title compound (5.65 g, 57%) as a pale pink oil.

LCMS m/z 170.1 (M+H)⁺ (ES⁺).

¹H NMR (DMSO-d₆) δ 6-95 (dd, J=11.8, 9.2 Hz, 1H), 6.60 (dd, J=13.3, 7.5 Hz, 1H), 5.27-5.23 (m, 1H), 5.02-4.97 (m, 1H), 4.95 (s, 2H), 1.97 (s, 3H).

### Step B: 4,5-difluoro-2-isopropylaniline

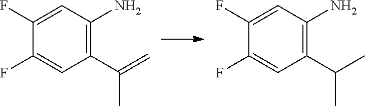

4,5-difluoro-2-(prop-1-en-2-yl)aniline (5.65 g, 33.4 mmol) and Pd/C (10%) (1.8 g, 10 wt %, 1.67 mmol) were suspended in EtOH (70 mL). The resulting mixture was stirred at RT under 5 atm H₂ for 6 h. The reaction mixture was filtered through a glass fibre filter and concentrated under reduced pressure to afford the title compound (5.21 g, 82%) as a light purple oil.

LCMS m/z 172.2 (M+H)⁺ (ES⁺).

¹H NMR (DMSO-d₆) δ 6.99 (dd, J=12.6, 9.2 Hz, 1H), 6.59 (dd, J=13.1, 7.6 Hz, 1H), 5.43 (s, 2H), 2.96-2.86 (m, 1H), 1.11 (d, J=6.8 Hz, 6H).

### Step C: 2-bromo-3,4-difluoro-6-isopropylaniline

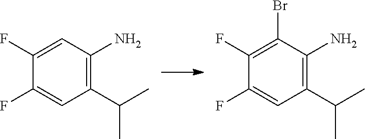

Prepared according to the general procedure of 2-bromo-4-fluoro-6-isopropylaniline (Intermediate B4, Step A) from 4,5-difluoro-2-isopropylaniline and NBS to afford the title compound (6.8 g, 83%) as a purple oil.

LCMS m/z 250.1/252.1 (M+H)⁺ (ES⁺).

¹H NMR (DMSO-d₆) δ 7.10 (dd, J=12.0, 8.9 Hz, 1H), 5.20 (s, 2H), 3.05 (sept, J=6.8 Hz, 1H), 1.13 (d, J=6.7 Hz, 6H).

### Step D: 3,4-difluoro-2-(2-fluoropyridin-4-yl)-6-isopropylaniline

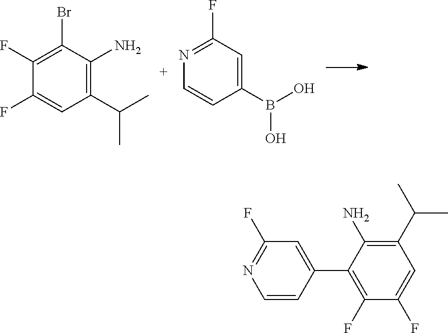

Prepared according to the general procedure of 4,5-difluoro-2-(prop-1-en-2-yl)aniline (Intermediate B7, Step A) from 2-bromo-3,4-difluoro-6-isopropylaniline and 2-fluoropyridine-4-boronic acid to afford the title compound (5.82 g, 76%) as an orange oil.

LCMS m/z 267.2 (M+H)⁺ (ES⁺).

¹H NMR (DMSO-d₆) δ 8.35 (d, J=5.1 Hz, 1H), 7.34 (d, J=4.5 Hz, 1H), 7.22 (s, 1H), 7.13 (dd, J=12.2, 9.0 Hz, 1H), 4.75 (s, 2H), 3.12-2.97 (m, 1H), 1.16 (d, J=6.7 Hz, 6H).

### Step E: 4-(2-bromo-5,6-difluoro-3-isopropylphenyl)-2-fluoropyridine

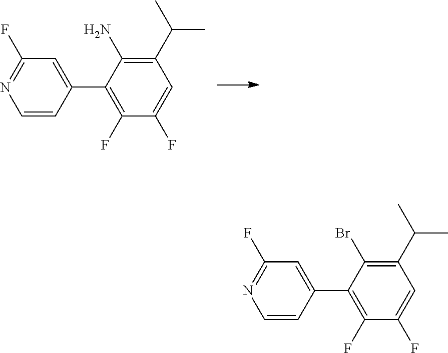 [SMILES: CC(C)c1cc(F)c(F)c(-c2ccnc(F)c2)c1N ; CC(C)c1cc(F)c(F)c(-c2ccnc(F)c2)c1Br]

Prepared according to the general procedure of 4-(2-bromo-5-fluoro-3-isopropyl-phenyl)-2-fluoropyridine (Intermediate B4, Step C) from 3,4-difluoro-2-(2-fluoro-pyridin-4-yl)-6-isopropylaniline to afford the title compound (4.52 g, 60%) as a light pink solid.

LCMS m/z 330.1/332.1 (M+H)⁺ (ES⁺).

¹H NMR (DMSO-d₆) δ 8.41 (d, J=5.1 Hz, 1H), 7.69 (dd, J=12.0, 8.4 Hz, 1H), 7.44-7.41 (m, 1H), 7.36 (s, 1H), 1.23 (d, J=6.8 Hz, 6H). One proton obscured by water signal.

### Step F: tert-butyl 2-(3,4-difluoro-2-(2-fluoropyridin-4-yl)-6-isopropylphenyl)acetate

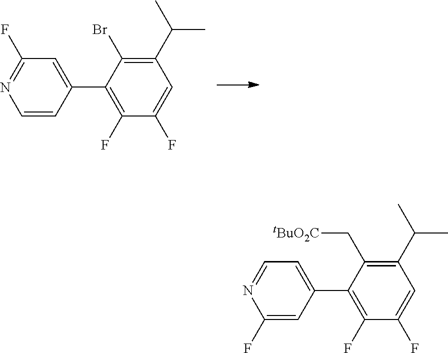 [SMILES: CC(C)c1cc(F)c(F)c(-c2ccnc(F)c2)c1Br ; CC(C)c1cc(F)c(F)c(-c2ccnc(F)c2)c1CC(=O)OC(C)(C)C]

Prepared according to the general procedure of tert-butyl 2-(4-fluoro-2-(2-fluoro-pyridin-4-yl)-6-isopropylphenyl)acetate (Intermediate B4, Step D) from 4-(2-bromo-5,6-difluoro-3-isopropylphenyl)-2-fluoropyridine and (2-isopropoxy-2-oxoethyl)zinc(II) bromide to afford the title compound (4.41 g, 83%) as an orange oil.

¹H NMR (DMSO-d₆) δ 8.39 (d, J=5.1 Hz, 1H), 7.53 (dd, J=12.3, 8.2 Hz, 1H), 7.30 (d, J=5.2 Hz, 1H), 7.16 (s, 1H), 3.47-3.36 (m, 2H), 3.11-3.01 (m, 1H), 1.31 (s, 9H), 1.19 (d, J=6.7 Hz, 6H).

### Intermediate B8: tert-butyl 2-(3,4-difluoro-6-(2-fluoropyridin-4-yl)-2-isopropyl-phenyl)acetate

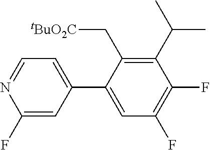 [SMILES: CC(C)c1c(F)c(F)cc(-c2ccnc(F)c2)c1CC(=O)OC(C)(C)C]

### Step A: 4,5-difluoro-2-(2-fluoropyridin-4-yl)aniline

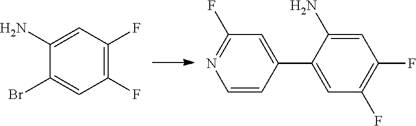 [SMILES: Nc1cc(F)c(F)cc1Br ; Nc1cc(F)c(F)cc1-c1ccnc(F)c1]

Prepared according to the general procedure of 4,5-difluoro-2-(prop-1-en-2-yl)aniline (Intermediate B7, Step A) from 2-bromo-4,5-difluoroaniline and 2-fluoropyridine-4-boronic acid to afford the title compound (6.91 g, 63%) as a pale orange solid.

LCMS m/z 225.1 (M+H)⁺ (ES⁺).

¹H NMR (DMSO-d₆) δ 8.27 (d, J=5.2 Hz, 1H), 7.42 (dt, J=5.2, 1.8 Hz, 1H), 7.25-7.17 (m, 2H), 6.74 (dd, J=13.2, 7.4 Hz, 1H), 5.33 (s, 2H).

### Step B: 2-bromo-3,4-difluoro-6-(2-fluoropyridin-4-yl)aniline

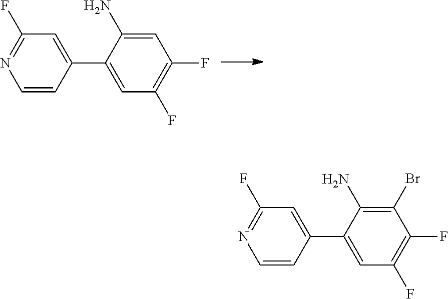 [SMILES: Nc1cc(F)c(F)cc1-c1ccnc(F)c1 ; Nc1c(-c2ccnc(F)c2)cc(F)c(F)c1Br]

Prepared according to the general procedure of 2-bromo-4-fluoro-6-isopropylaniline (Intermediate B4, Step A) from 4,5-difluoro-2-(2-fluoropyridin-4-yl)aniline and NBS to afford the title compound (7.72 g, 79%) as a brown solid.

LCMS m/z 303.0/305.0 (M+H)⁺ (ES⁺).

¹H NMR (DMSO-d₆) δ 8.31 (d, J=5.2 Hz, 1H), 7.42-7.38 (m, 1H), 7.32 (dd, J=10.9, 8.7 Hz, 1H), 7.25-7.20 (m, 1H), 5.32 (br s, 2H).

### Step C: 3,4-difluoro-6-(2-fluoropyridin-4-yl)-2-(prop-1-en-2-yl)aniline

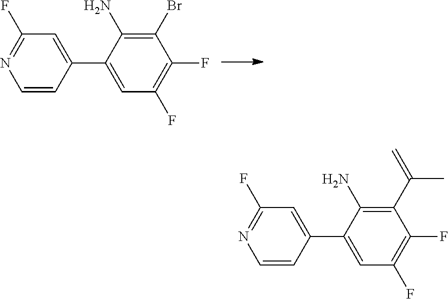 [SMILES: Nc1c(-c2ccnc(F)c2)cc(F)c(F)c1Br ; C=C(C)c1c(N)c(-c2ccnc(F)c2)cc(F)c1F]

Prepared according to the general procedure of 4,5-difluoro-2-(prop-1-en-2-yl)aniline (Intermediate B7, Step A) from 2-bromo-3,4-difluoro-6-(2-fluoropyridin-4-yl)-aniline and isopropenylboronic acid pinacol ester to afford the title compound (7.11 g, 95%) as a pale orange solid.

LCMS m/z 265.2 (M+H)⁺ (ES⁺).

¹H NMR (DMSO-d₆) δ 8.28 (d, J=5.2 Hz, 1H), 7.44-7.39 (m, 1H), 7.26-7.21 (m, 1H), 7.17 (dd, J=11.2, 8.8 Hz, 1H), 5.54-5.50 (m, 1H), 5.11-5.06 (m, 1H), 4.74 (s, 2H), 2.00 (s, 3H).

### Step D: 3,4-difluoro-6-(2-fluoropyridin-4-yl)-2-isopropylaniline

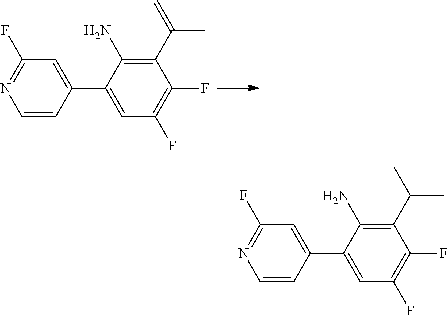 [SMILES: C=C(C)c1c(N)c(-c2ccnc(F)c2)cc(F)c1F ; CC(C)c1c(N)c(-c2ccnc(F)c2)cc(F)c1F]

Prepared according to the general procedure of 4,5-difluoro-2-isopropylaniline (Intermediate B7, Step B) from 3,4-difluoro-6-(2-fluoropyridin-4-yl)-2-(prop-1-en-2-yl)aniline to afford the title compound (6.52 g, 97%) as an orange oil.

LCMS m/z 267.3 (M+H)⁺ (ES⁺).

¹H NMR (DMSO-d₆) δ 8.27 (d, J=5.2 Hz, 1H), 7.38 (dt, J=5.2, 1.8 Hz, 1H), 7.22-7.16 (m, 1H), 7.02 (dd, J=11.0, 8.8 Hz, 1H), 4.90 (s, 2H), 3.27 (p, J=7.1 Hz, 1H), 1.29 (dd, J=7.0, 1.6 Hz, 6H).

### Step E: 4-(2-bromo-4,5-difluoro-3-isopropylphenyl)-2-fluoropyridine

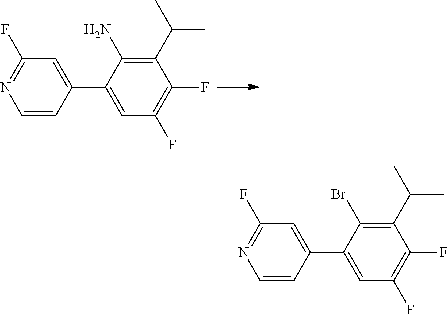 [SMILES: CC(C)c1c(N)c(-c2ccnc(F)c2)cc(F)c1F ; CC(C)c1c(F)c(F)cc(-c2ccnc(F)c2)c1Br]

Prepared according to the general procedure of 4-(2-bromo-5-fluoro-3-isopropyl-phenyl)-2-fluoropyridine (Intermediate B4, Step C) from 3,4-difluoro-6-(2-fluoro-pyridin-4-yl)-2-isopropylaniline to afford the title compound (4.52 g, 60%) as a pale yellow oil.

LCMS m/z 330.1/332.1 (M+H)⁺ (ES⁺).

¹H NMR (DMSO-d₆) δ 8.34 (d, J=5.1 Hz, 1H), 7.56 (dd, J=10.6, 8.2 Hz, 1H), 7.40 (dt, J=5.1, 1.7 Hz, 1H), 7.27 (s, 1H), 3.63 (tt, J=8.2, 6.3 Hz, 1H), 1.36 (dd, J=7.1, 1.7 Hz, 6H).

### Step F: tert-butyl 2-(3,4-difluoro-6-(2-fluoropyridin-4-yl)-2-isopropylphenyl)acetate

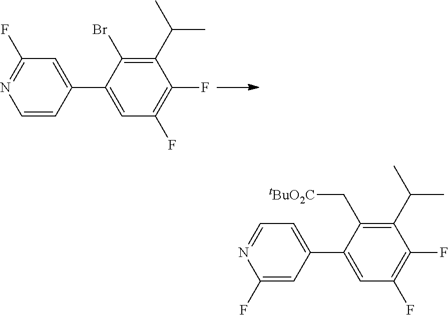 [SMILES: CC(C)c1c(F)c(F)cc(-c2ccnc(F)c2)c1Br ; CC(C)c1c(F)c(F)cc(-c2ccnc(F)c2)c1CC(=O)OC(C)(C)C]

Prepared according to the general procedure of tert-butyl 2-(4-fluoro-2-(2-fluoro-pyridin-4-yl)-6-isopropylphenyl)acetate (Intermediate B4, Step D) from 4-(2-bromo-4,5-difluoro-3-isopropylphenyl)-2-fluoropyridine and (2-isopropoxy-2-oxoethyl)zinc(II) bromide to afford the title compound (5.62 g, 88%) as an orange oil.

¹H NMR (DMSO-d₆) δ 8.33 (d, J=5.1 Hz, 1H), 7.30 (dd, J=10.6, 8.0 Hz, 1H), 7.27-7.22 (m, 1H), 7.07 (s, 1H), 3.50 (s, 2H), 3.12 (p, J=6.9 Hz, 1H), 1.37 (s, 9H), 1.34-1.29 (m, 6H).

### Intermediate B9: 5-(2-bromo-3-isopropylphenyl)-3-chloropyridazine

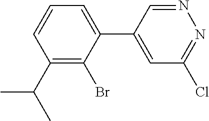 [SMILES: CC(C)c1cccc(-c2cnnc(Cl)c2)c1Br]

### Step A: 2-chloro-6-(prop-1-en-2-yl)aniline

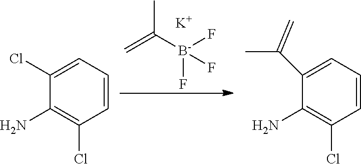

To a solution of 2,6-dichloroaniline (40 g, 247 mmol) in dioxane (400 mL) and H₂O (40 mL) was added potassium trifluoro(prop-1-en-2-yl)borate (38.4 g, 259 mmol), K₂CO₃ (68.2 g, 494 mmol) and Pd(dppf)Cl₂.DCM (10.1 g, 12.3 mmol) under N₂. The mixture was warmed to 90° C. and stirred at 90° C. for 12 h. The reaction mixture was quenched with H₂O (300 mL) and extracted with EtOAc (300 mL×3). The combined organic phases were washed with brine (300 mL×2), dried over anhydrous Na₂SO₄ and filtered. The filtrate was concentrated in vacuum. The residue was purified by FC (PE:EtOAc, 1:0 to 1:0) to give the title compound (25 g, 60.4%) as a yellow oil.

LCMS: m/z 168.1 (M+H)⁺ (ES⁺).

¹H NMR (DMSO-d₆) δ 7.14 (dd, 1H), 8.90 (dd, 1H), 6.57 (t, 1H), 5.28 (t, 1H), 4.99 (d, 1H), 4.84 (br s, 2H), 2.00 (s, 3H).

### Step B: 2-(prop-1-en-2-yl)-6-(4,4,5,5-tetramethyl-1,3,2-dioxaborolan-2-yl)aniline

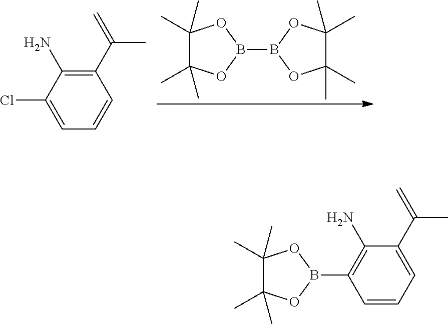

To a solution of 2-chloro-6-(prop-1-en-2-yl)aniline (15 g, 89.5 mmol), 4,4,4′,4′,5,5,5′,5′-octamethyl-2,2′-bi(1,3,2-dioxaborolane) (34.1 g, 134 mmol) and AcOK (26.4 g, 268 mmol) in dioxane (250 mL) was added XPhos (3.41 g, 7.16 mmol) and Pd₂(dba)₃ (3.28 g, 3.58 mmol) at 20° C. under N₂. The reaction mixture was warmed to 100° C. and stirred at 100° C. for 12 h. The reaction mixture was quenched with H₂O (300 mL) and extracted with EtOAc (300 mL×3). The organic phases were washed with brine (300 mL×2), dried over anhydrous Na₂SO₄ and filtered. The filtrate was concentrated in vacuum. The residue was purified by FC (PE:EtOAc, 1:0 to 100:1) to give the title compound (23 g, 76.3%) as a yellow oil.

LCMS: m/z 260.2 (M+H)⁺ (ES⁺).

¹H NMR (DMSO-d₆) δ 7.33 (dd, 1H), 7.00 (dd, 1H), 6.51 (t, 1H), 5.23 (t, 1H), 5.18 (br s, 2H), 4.93 (s, 1H), 1.99 (s, 3H), 1.29 (s, 12H).

### Step C: 5-(2-amino-3-(prop-1-en-2-yl)phenyl)pyridazin-3-ol

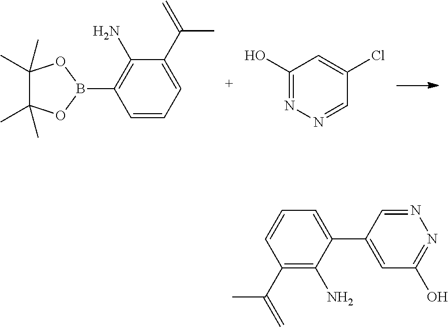 [SMILES: C=C(C)c1cccc(B2OC(C)(C)C(C)(C)O2)c1N ; Oc1cc(Cl)cnn1 ; C=C(C)c1cccc(-c2cnnc(O)c2)c1N]

To a solution of 2-(prop-1-en-2-yl)-6-(4,4,5,5-tetramethyl-1,3,2-dioxaborolan-2-yl)-aniline (3 g, 11.6 mmol) and 5-chloropyridazin-3-ol (1.51 g, 11.6 mmol) in dioxane (30 mL) and H₂O (4 mL) was added Pd(dppf)Cl₂.DCM (473 mg, 579 μmol) and K₂CO₃ (3.20 g, 23.2 mmol) under N₂. The reaction mixture was heated to 100° C. and stirred at 100° C. for 1 h. The reaction mixture was quenched with H₂O (80 mL) and extracted with EtOAc (80 mL×3). The combined organic phases were washed with brine (80 mL×2), dried over anhydrous Na₂SO₄ and filtered. The filtrate was concentrated in vacuum. The residue was purified by FC (PE:EtOAc, 2:1 to 1:1) to give the title compound (2.1 g, 72.6%) as a yellow oil.

LCMS: m/z 228.2 (M+H)⁺ (ES⁺).

¹H NMR (DMSO-d₆) δ 12.98 (br s, 1H), 7.90 (d, 1H), 7.04-6.98 (m, 2H), 6.81 (s, 1H), 6.68 (t, 1H), 5.30 (d, 1H), 5.00 (d, 1H), 4.76 (br s, 2H), 2.02 (s, 3H).

### Step D: 5-(2-amino-3-isopropylphenyl)pyridazin-3-ol

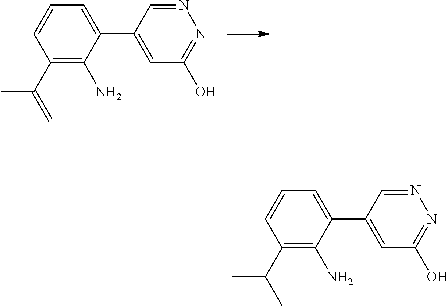 [SMILES: C=C(C)c1cccc(-c2cnnc(O)c2)c1N ; CC(C)c1cccc(-c2cnnc(O)c2)c1N]

To a solution of 5-(2-amino-3-(prop-1-en-2-yl)phenyl)pyridazin-3-ol (2.1 g, 9.24 mmol) in MeOH (25 mL) was added Pd/C (0.1 g, 10 wt % on charcoal) under N₂. The suspension was degassed under reduced pressure and purged with H₂ several times. The reaction mixture was stirred at 25° C. for 36 h under H₂ (15 psi). The reaction mixture was filtered and the filtrate was concentrated in vacuum to give the title compound (1.9 g, 89.7%) as a green solid.

LCMS: m/z 230.1 (M+H)⁺ (ES⁺).

¹H NMR (DMSO-d₆) δ 12.94 (br s, 1H), 7.87 (d, 1H), 7.12 (d, 1H), 6.90 (t, 1H), 6.78 (d, 1H), 6.67 (t, 1H), 4.89 (br s, 2H), 3.10-3.02 (m, 1H), 1.17 (d, 6H).

### Step E: 5-(2-bromo-3-isopropylphenyl)pyridazin-3-ol

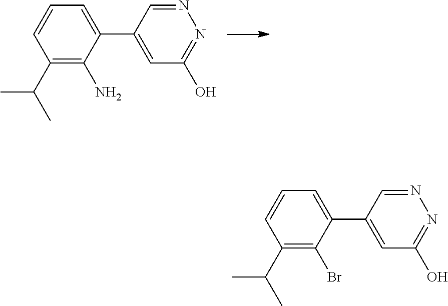 [SMILES: CC(C)c1cccc(-c2cnnc(O)c2)c1N ; CC(C)c1cccc(-c2cnnc(O)c2)c1Br]

To a solution of 5-(2-amino-3-isopropylphenyl)pyridazin-3-ol (1.3 g, 5.67 mmol) in MeCN (26 mL) was added a solution of HBr (3.87 g, 17.7 mmol, 37 wt % in AcOH) in H₂O (2.6 mL) at 0° C., followed by a solution of NaNO₂ (469 mg, 6.80 mmol) in H₂O (2.6 mL) at 0° C. Then the resulting mixture was stirred at 0° C. for 0.5 h. To the above solution was added CuBr (976 mg, 6.80 mmol) and CuBr₂ (507 mg, 2.27 mmol). The reaction mixture was stirred at 25° C. for 14 h. The reaction mixture was quenched with H₂O (50 mL) and extracted with EtOAc (50 mL×3). The combined organic phases were washed with brine (50 mL×2), dried over anhydrous Na₂SO₄ and filtered. The filtrate was concentrated in vacuum. The residue was purified by FC (PE:EtOAc, 3:1 to 2:1) to give the title compound (1.2 g, 72.2%) as a yellow solid.

LCMS: m/z 295.0 (M+H)⁺ (ES⁺).

¹H NMR (DMSO-d₆) δ 13.17 (br s, 1H), 7.91 (s, 1H), 7.51-7.44 (m, 2H), 7.32-7.25 (m, 1H), 6.83 (s, 1H), 3.43-3.37 (m, 1H), 1.31 (d, 6H).

### Step F: 5-(2-bromo-3-isopropylphenyl)-3-chloropyridazine

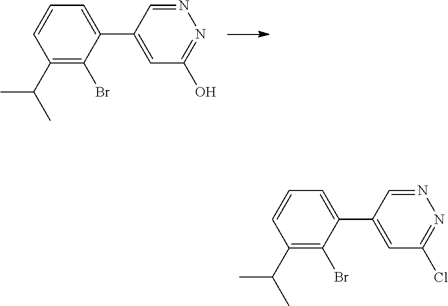 [SMILES: CC(C)c1cccc(-c2cnnc(O)c2)c1Br ; CC(C)c1cccc(-c2cnnc(Cl)c2)c1Br]

A solution of 5-(2-bromo-3-isopropylphenyl)pyridazin-3-ol (1.2 g, 4.09 mmol) in POCl₃ (19.8 g, 129 mmol) was heated to 80° C. and stirred at 80° C. for 1 h. The reaction mixture was slowly quenched with H₂O (150 mL) at 20° C. The mixture was adjusted to pH=7 with solid NaOH at 25° C. and then extracted with EtOAc (150 mL×3). The combined organic phases were washed with brine (150 mL×2), dried over anhydrous Na₂SO₄ and filtered. The filtrate was concentrated in vacuum. The residue was purified by FC (PE:EtOAc, 8:1 to 5:1) to give the title compound (1 g, 78.4%) as a yellow solid.

LCMS: m/z 312.9 (M+H)⁺ (ES⁺).

¹H NMR (CDCl₃) δ 9.19 (s, 1H), 7.57 (d, 1H), 7.46-7.40 (m, 2H), 7.13-7.10 (m, 1H), 3.54-3.47 (m, 1H), 1.30 (d, 6H).

### Intermediate B10: tert-butyl 2-(4-cyano-2-(2-fluoropyridin-4-yl)-6-isopropyl-phenyl)acetate

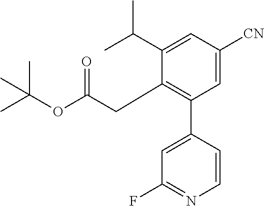 [SMILES: CC(C)c1cc(C#N)cc(-c2ccnc(F)c2)c1CC(=O)OC(C)(C)C]

### Step A: 4-amino-3-(prop-1-en-2-yl)benzonitrile

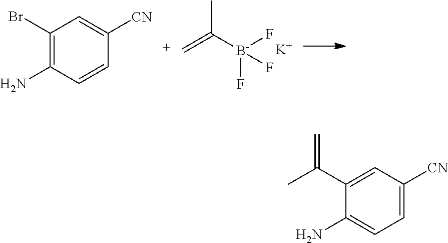

To a solution of 4-amino-3-bromobenzonitrile (6 g, 30.5 mmol) in dioxane (60 mL) and H₂O (12 mL) was added potassium trifluoro(prop-1-en-2-yl)borate (5-41 g, 36.5 mmol), Pd(dppf)Cl₂.DCM (1.24 g, 1.52 mmol) and Na₂CO₃ (6.46 g, 60.9 mmol). The mixture was stirred at 90° C. for 12 h under N₂. The reaction mixture was poured into water (100 mL) and extracted with EtOAc (150 mL×3). The organic layers were dried over anhydrous Na₂SO₄, filtered and concentrated in vacuum. The residue was purified by FC (PE:EtOAc, 30:1 to 10:1) to give the title compound (4 g, 82.5% yield, 99.3% purity on LCMS) as a yellow oil.

LCMS: m/z 159.2 (M+H)⁺ (ES⁺).

¹H NMR (CDCl₃): δ 7.35-7.28 (m, 2H), 6.67 (d, 1H), 5.37 (d, 1H), 5.09 (s, 1H), 4.33 (br s, 2H), 2.05 (s, 3H).

### Step B: 4-amino-3-isopropylbenzonitrile

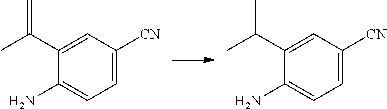

To a solution of 4-amino-3-(prop-1-en-2-yl)benzonitrile (4 g, 25.3 mmol) in MeOH (50 mL) was added Pd/C (0.5 g, 10% purity loading on active carbon) under N₂. The mixture was degassed in vacuum and purged with H₂ several times. The mixture was stirred at 25° C. for 12 h under H₂ (15 psi). The reaction mixture was filtered through the celite and the filtrate was concentrated in vacuum to give the title compound (4 g, 98.7% yield) as a yellow oil.

LCMS: m/z 161.2 (M+H)⁺ (ES⁺).

¹H NMR (CDCl₃): δ 7.40 (d, 1H), 7.30 (d, 1H), 6.65 (d, 1H), 4.13 (br s, 2H), 2.86-2.79 (m, 1H), 1.27 (d, 6H).

### Step C: 4-amino-3-bromo-5-isopropylbenzonitrile

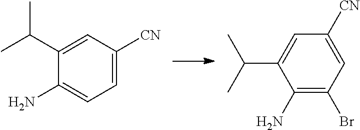

To a solution of 4-amino-3-isopropylbenzonitrile (4 g, 25.0 mmol) in MeCN (50 mL) was added NBS (4.67 g, 26.2 mmol). The mixture was stirred at 25° C. for 1 h, then quenched with sat aq Na₂SO₃ (50 mL) and extracted with EtOAc (100 mL×3). The organic layers were dried over anhydrous Na₂SO₄, filtered and concentrated in vacuum. The residue was purified by FC (PE:EtOAc, 5:1 to 3:1) to give the title compound (5.4 g, 90.5% yield) as a yellow solid.

LCMS: m/z 240.8 (M+H)⁺ (ES⁺).

¹H NMR (CDCl₃): 7.60 (d, 1H), 7.34 (d, 1H), 4.70 (br s, 2H), 2.97-2.78 (m, 1H), 1.28 (d, 6H).

### Step D: 4-amino-3-(2-fluoropyridin-4-yl)-5-isopropylbenzonitrile

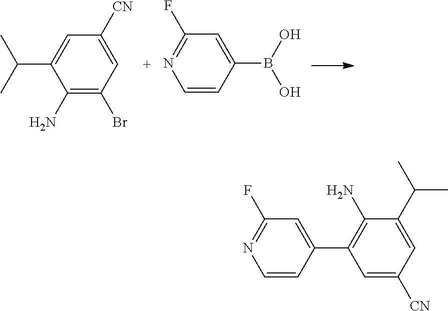

To a solution of 4-amino-3-bromo-5-isopropylbenzonitrile (4.9 g, 20.5 mmol) and (2-fluoropyridin-4-yl)boronic acid (3.03 g, 21.5 mmol) in dioxane (10 mL) and H₂O (2 mL) was added Pd(dppf)Cl₂.DCM (837 mg, 1.02 mmol) and Na₂CO₃ (4.34 g, 41.0 mmol). The mixture was stirred at 90° C. for 12 h under N₂. The reaction mixture was poured into water (100 mL) and extracted with EtOAc (150 mL×3). The organic layers were dried over anhydrous Na₂SO₄, filtered and concentrated in vacuum. The residue was purified by FC (PE:EtOAc, 10:1 to 3:1) to give the title compound (5 g, 95.6% yield) as a yellow solid.

LCMS: m/z 256.3 (M+H)⁺ (ES⁺).

¹H NMR (CDCl₃): δ 8.35 (d, 1H), 7.47 (d, 1H), 7.28-7.26 (m, 2H), 7.03 (s, 1H), 4.31 (br s, 2H), 2.90-2.84 (m, 1H), 1.32 (d, 6H).

### Step E: 4-bromo-3-(2-fluoropyridin-4-yl)-5-isopropylbenzonitrile

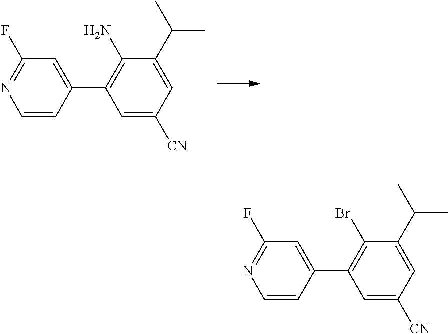 [SMILES: CC(C)c1cc(C#N)cc(-c2ccnc(F)c2)c1N ; CC(C)c1cc(C#N)cc(-c2ccnc(F)c2)c1Br]

To a solution of 4-amino-3-(2-fluoropyridin-4-yl)-5-isopropylbenzonitrile (5 g, 19.6 mmol) in MeCN (100 mL) was added HBr (73-7 mmol, 10 mL, 40% purity in H₂O) in H₂O (1 mL) at 0° C. Then a solution of NaNO₂ (1.62 g, 23.5 mmol) in H₂O (1 mL) was added. The mixture was stirred at 0° C. for 30 min, then CuBr₂ (2.19 g, 9.79 mmol) and CuBr (140 mg, 979 μmol) were added. The mixture was stirred at 25° C. for 2 h, then quenched with sat aq Na₂SO₃ (100 mL) and extracted with EtOAc (150 mL×3). The organic layers were dried over anhydrous Na₂SO₄, filtered and concentrated in vacuum. The residue was purified by FC (PE:EtOAc, 30: 1 to 10:1) to give the title compound (5.9 g, 94.4% yield) as a yellow solid.

LCMS: m/z 319.2, 321.2 (M+H)⁺ (ES⁺).

¹H NMR (CDCl₃): δ 8.33 (d, 1H), 7.63 (d, 1H), 7.38 (d, 1H), 7.18 (dd, 1H), 6.94 (s, 1H), 3.56-3.50 (m, 1H), 1.32 (d, 6H).

### Step F: tert-butyl 2-(4-cyano-2-(2-fluoropyridin-4-yl)-6-isopropylphenyl)acetate

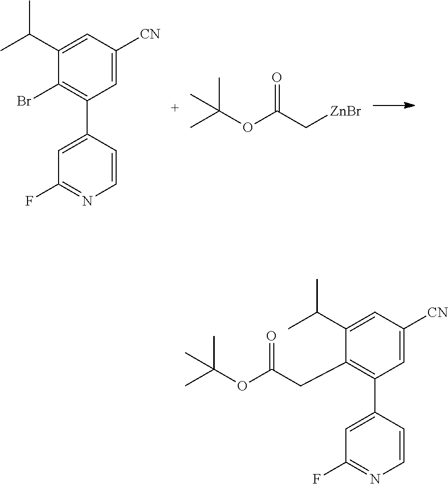

To a solution of 4-bromo-3-(2-fluoropyridin-4-yl)-5-isopropylbenzonitrile (5.8 g, 18.2 mmol) and palladium; tri-tert-butylphosphane (464 mg, 909 μmol) in THF (50 mL) was added (2-(tert-butoxy)-2-oxoethyl)zinc(II) bromide (0.51 M in THF, 107 mL) under N₂. The mixture was stirred at 70° C. for 2 h, then quenched with water (150 mL) and extracted with EtOAc (200 mL×3). The organic layers were dried over anhydrous Na₂SO₄, filtered and concentrated in vacuum. The residue was purified by reverse phase flash chromatography (water (0.1% HCl)-MeCN) to give the title compound (4 g, 54.6% yield, 97% purity on LCMS, HCl salt) as a yellow solid.

LCMS: m/z 355.4 (M−HCl+H)⁺ (ES⁺).

¹H NMR (CD₃OD): δ 8.29 (d, 1H), 7.81 (d, 1H), 7.49 (d, 1H), 7.28-7.26 (m, 1H), 7.05 (s, 1H), 3.64 (s, 2H), 3.25-3.14 (m, 1H), 1.41 (s, 9H), 1.30 (d, 6H). HCl proton not observed.

### Intermediate B11: tert-butyl 2-(4-(difluoromethoxy)-2-(2-fluoropyridin-4-yl)-6-isopropylphenyl)acetate

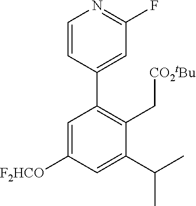 [SMILES: CC(C)c1cc(OC(F)F)cc(-c2ccnc(F)c2)c1CC(=O)OC(C)(C)C]

### Step A: 2-bromo-4-(difluoromethoxy)aniline

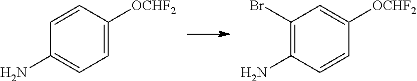 [SMILES: Nc1ccc(OC(F)F)cc1 ; Nc1ccc(OC(F)F)cc1Br]

To a solution of 4-(difluoromethoxy)aniline (20 g, 126 mmol) in MeCN (200 mL) was added dropwise a solution of NBS (20.1 g, 113 mmol) in MeCN (200 mL) at 0° C. The mixture was stirred at 25° C. for 3 h. The reaction mixture was diluted with H₂O (250 mL) and extracted with DCM (250 mL×3). The combined organic phases were washed with brine (250 mL), dried over anhydrous Na₂SO₄, filtered and concentrated in vacuum. The residue was purified by FC (PE:EtOAc, 200:1 to 20:1) to give the title compound (24 g, 80.2% yield) as a yellow oil.

LCMS: m/z 238.03 (M+H)⁺ (ES⁺).

¹H NMR (CDCl₃): δ 7.25 (s, 1H), 6.94 (dd, 1H), 6.74 (d, 1H), 6.38 (t, 1H), 4.10 (br s, 2H).

### Step B: 4-(difluoromethoxy)-2-(prop-1-en-2-yl)aniline

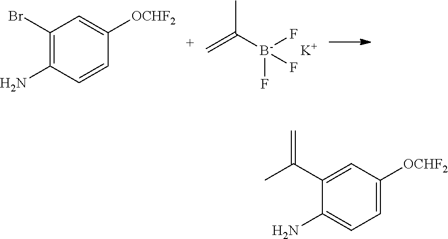

To a solution of 2-bromo-4-(difluoromethoxy)aniline (10 g, 42.0 mmol) and potassium trifluoro(prop-1-en-2-yl)borate (6.84 g, 46.2 mmol) in dioxane (200 mL) and H₂O (40 mL) were added Pd(dppf)Cl₂.DCM (3.43 g, 4.20 mmol) and Na₂CO₃ (11.1 g, 105 mmol). The mixture was stirred at 95° C. for 12 h under N₂. The reaction mixture was diluted with H₂O (300 mL) and extracted with DCM (300 mL×3). The combined organic phases were washed with brine (300 mL), dried over anhydrous Na₂SO₄, filtered and concentrated in vacuum. The residue was purified by FC (PE:EtOAc, 1:0 to 20:1) to give the title compound (7 g, 83.7% yield) as a yellow oil.

LCMS: m/z 200.2 (M+H)⁺ (ES⁺).

¹H NMR (CDCl₃): δ 6.84 (d, 2H), 6.64 (d, 1H), 6.39 (t, 1H), 5.32 (s, 1H), 5.08 (s, 1H), 3.80 (br s, 2H), 2.06 (s, 3H).

### Step C: 4-(difluoromethoxy)-2-isopropylaniline

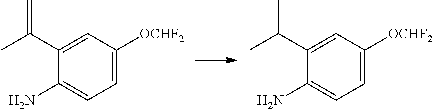

To a solution of 4-(difluoromethoxy)-2-(prop-1-en-2-yl)aniline (7 g, 35.1 mmol) in MeOH (200 mL) was added Pd/C (2 g, 10% purity loading on active carbon) under N₂. The mixture was degassed under vacuum and purged three times with H₂. The mixture was stirred at 25° C. for 12 h under H₂ (15 psi). The reaction mixture was filtered and diluted with H₂O (300 mL) and extracted with EtOAc (300 mL×2). The combined organic phases were washed with brine (300 mL), dried over anhydrous Na₂SO₄, filtered and concentrated in vacuum. The residue was purified by FC (PE:EtOAc, 1:0 to 10:1) to give the title compound (7 g, 99.0% yield) as a yellow solid.

LCMS: m/z 202.1 (M+H)⁺ (ES⁺).

¹H NMR (CDCl₃): δ 6.92 (d, 1H), 6.82 (dd, 1H), 6.60 (d, 1H), 6.39 (t, 1H), 3.52 (br s, 2H), 2.92-2.85 (m, 1H), 1.25 (d, 6H).

### Step D: 2-bromo-4-(difluoromethoxy)-6-isopropylaniline

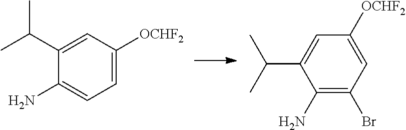

To a solution of 4-(difluoromethoxy)-2-isopropylaniline (6.9 g, 34.3 mmol) in MeCN (300 mL) was added dropwise a solution of NBS (6.1 g, 34.3 mmol) in MeCN (300 mL) at 0° C. The mixture was stirred at 25° C. for 3 h. The reaction solution was diluted with H₂O (300 ml) and extracted with DCM (300 mL×3). The combined organic phases were washed with brine (300 mL), dried over anhydrous Na₂SO₄, filtered and concentrated in vacuum. The residue was purified by FC (PE:EtOAc, 100:1 to 10:1) to give the title compound (9 g, 93.7% yield) as a grey oil.

LCMS: m/z 280.1 (M+H)⁺ (ES⁺).

¹H NMR (CDCl₃): δ 7.15 (d, 1H), 6.91 (d, 1H), 6.38 (t, 1H), 4.12 (br s, 2H), 2.93-2.86 (m, 1H), 1.25 (d, 6H).

### Step E: 4-(difluoromethoxy)-2-(2-fluoropyridin-4-yl)-6-isopropylaniline

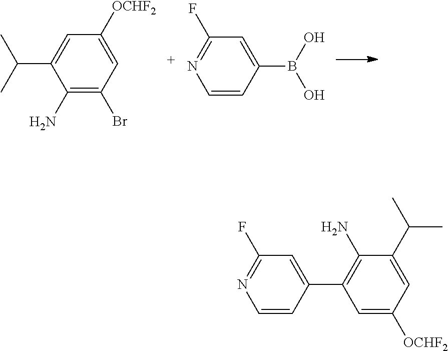

To a solution of (2-fluoropyridin-4-yl)boronic acid (4.70 g, 33.4 mmol) and 2-bromo-4-(difluoromethoxy)-6-isopropylaniline (8.5 g, 30.4 mmol) in H₂O (60 mL) and dioxane (240 mL) were added Pd(dppf)Cl₂.DCM (2.48 g, 3.03 mmol) and Na₂CO₃ (8.04 g, 75.9 mmol). The mixture was stirred at 95° C. for 12 h under N₂, then diluted with H₂O (600 mL) and extracted with DCM (600 mL×3). The combined organic phases were washed with brine (600 mL), dried over anhydrous Na₂SO₄, filtered and concentrated in vacuum. The residue was purified by FC (PE:EtOAc, 20:1 to 10:1) to give the title compound (8.2 g, 91.2% yield) as a yellow solid.

LCMS: m/z 296.9 (M+H)⁺ (ES⁺).

¹H NMR (CDCl₃): δ 8.31 (d, 1H), 7.31 (d, 1H), 7.05 (s, 1H), 7.02 (d, 1H), 7.80 (d, 1H), 6.43 (t, 1H), 3.76 (br s, 2H), 2.95-2.88 (m, 1H), 1.28 (d, 6H).

### Step F: 4-(2-bromo-5-(difluoromethoxy)-3-isopropylphenyl)-2-fluoropyridine

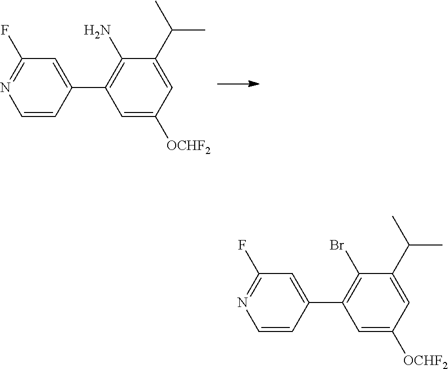 [SMILES: CC(C)c1cc(OC(F)F)cc(-c2ccnc(F)c2)c1N ; CC(C)c1cc(OC(F)F)cc(-c2ccnc(F)c2)c1Br]

To a solution of 4-(difluoromethoxy)-2-(2-fluoropyridin-4-yl)-6-isopropylaniline (7.5 g, 25.3 mmol) in MeCN (200 mL) was added 3-methylbutyl nitrite (3.56 g, 30.4 mmol) at 0° C., followed by CuBr (4.36 g, 30.4 mmol) at 0° C. Then the resulting mixture was stirred at 60° C. for 1 h, then quenched with sat aq NaHCO₃ (300 mL) and extracted with EtOAc (300 mL×3). The combined organic phases were washed with brine (300 mL), dried over anhydrous Na₂SO₄, filtered and concentrated in vacuum. The residue was purified by FC (PE:EtOAc, 1:0 to 10:1) to give the title compound (5 g, 54.8% yield) as a yellow oil.

LCMS: m/z 361.8 (M+H)⁺ (ES⁺).

¹H NMR (CDCl₃): δ 8.29 (d, 1H), 7.20 (dd, 1H), 7.14 (d, 1H), 6.94 (s, 1H), 6.90 (d, 1H), 6.54 (t, 1H), 3.54-3.46 (m, 1H), 1.28 (d, 6H).

### Step G: tert-butyl 2-(4-(difluoromethoxy)-2-(2-fluoropyridin-4-yl)-6-isopropylphenyl)-acetate

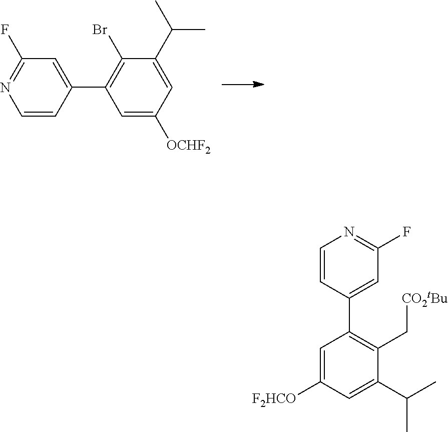 [SMILES: CC(C)c1cc(OC(F)F)cc(-c2ccnc(F)c2)c1Br ; CC(C)c1cc(OC(F)F)cc(-c2ccnc(F)c2)c1CC(=O)OC(C)(C)C]

To a solution of 4-(2-bromo-5-(difluoromethoxy)-3-isopropylphenyl)-2-fluoropyridine (2 g, 5.55 mmol) in THF (50 mL) was added dicyclohexyl-[2-(2,4,6-triisopropylphenyl)-phenyl]phosphane (265 mg, 555 μmol) and Pd₂(dba)₃ (254 mg, 278 μmol) under N₂. (2-(tert-butoxy)-2-oxoethyl)zinc(II) bromide (0.5 M in THF, 36.2 mL) was added and the reaction mixture was stirred at 70° C. for 12 h. The reaction mixture was diluted with sat aq NaHCO₃ (20 mL) and extracted with DCM (20 mL×3). The combined organic phases were washed with brine (20 mL), dried over anhydrous Na₂SO₄, filtered and concentrated in vacuum. The residue was purified by FC (PE:EtOAc, 95:5 to 95:5) to give the title compound (2.1 g, 95.6% yield) as a yellow oil.

LCMS: m/z 396.1 (M+H)⁺ (ES⁺).

¹H NMR (CDCl₃): δ 8.38 (d, 1H), 7.31 (s, 2H), 7.04 (s, 1H), 6.94 (s, 1H), 6.65 (t, 1H), 3.58 (s, 2H), 3.24-3.18 (m, 1H), 1.55 (s, 9H), 1.38 (d, 6H).

### Intermediate B12: tert-butyl 2-(4-cyano-3-fluoro-6-(2-fluoropyridin-4-yl)-2-isopropylphenyl)acetate

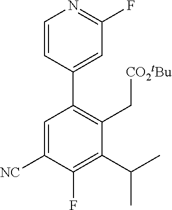 [SMILES: CC(C)c1c(F)c(C#N)cc(-c2ccnc(F)c2)c1CC(=O)OC(C)(C)C]

### Step A: 4-amino-5-bromo-2-fluorobenzonitrile

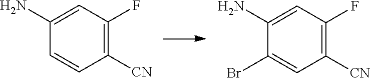 [SMILES: N#Cc1ccc(N)cc1F ; N#Cc1cc(Br)c(N)cc1F]

To a solution of 4-amino-2-fluorobenzonitrile (7 g, 51.4 mmol) in MeCN (200 mL) was added NBS (9.15 g, 51.4 mmol) in portions at 25° C. Then the solution was stirred at 50° C. for 3 h. The solution was quenched with H₂O (30 mL) and extracted with EtOAc (30 mL×2). The organic phases were washed with brine (40 mL), dried over anhydrous Na₂SO₄, filtered and concentrated in vacuum. The residue was purified by FC (PE:EtOAc, 5:1 to 4:1) to give the title compound (10.8 g, 97.7% yield) as a yellow solid.

LCMS: m/z 216.8 (M+H)⁺ (ES⁺).

¹H NMR (CDCl₃): δ 7.61 (d, 1H), 6.52 (d, 1H), 4.82 (br s, 2H).

### Step B: 4-amino-2-fluoro-5-(2-fluoropyridin-4-yl)benzonitrile

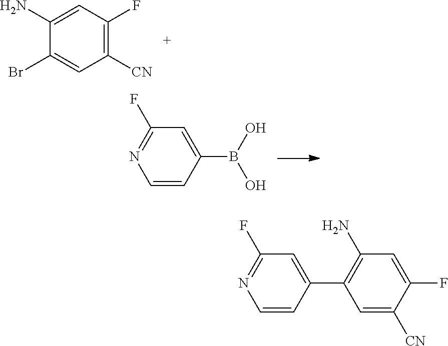

To a solution of 4-amino-5-bromo-2-fluorobenzonitrile (6 g, 27.9 mmol) and (2-fluoro-pyridin-4-yl)boronic acid (3.93 g, 27.9 mmol) in dioxane (100 mL) and H₂O (20 mL) was added Pd(dppf)Cl₂.DCM (1.14 g, 1.40 mmol) and Na₂CO₃ (7.39 g, 69.8 mmol) under N₂. The solution was stirred at 100° C. for 2 h under N₂, then quenched with H₂O (200 mL) and extracted with EtOAc (200 mL×2). The organic phases were washed with brine (400 mL), dried over anhydrous Na₂SO₄, filtered and concentrated in vacuum. The residue was purified by FC (PE:EtOAc, 2:1 to 1:1) to give the title compound (6.3 g, 97.7% yield) as a yellow solid.

LCMS: m/z 232.1 (M+H)⁺ (ES⁺).

¹H NMR (CD₃OD): δ 8.29 (d, 1H), 7.44 (d, 1H), 7.38 (dd, 1H), 7.17 (s, 1H), 6.64 (d, 1H). Two exchangeable protons not observed.

### Step C: 4-amino-3-bromo-2-fluoro-5-(2-fluoropyridin-4-yl)benzonitrile

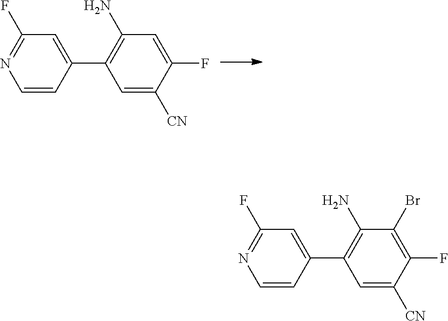 [SMILES: N#Cc1cc(-c2ccnc(F)c2)c(N)cc1F ; N#Cc1cc(-c2ccnc(F)c2)c(N)c(Br)c1F]

To a solution of 4-amino-2-fluoro-5-(2-fluoropyridin-4-yl)benzonitrile (6 g, 26.0 mmol) in MeCN (120 mL) was added NBS (4.62 g, 26.0 mmol) and the solution was stirred at 25° C. for 1 h. The solution was quenched with sat aq Na₂SO₃ (10 mL) and extracted with DCM (10 mL×3). The organic phases were washed with brine (15 mL), dried over anhydrous Na₂SO₄, filtered and concentrated in vacuum. The residue was triturated with a mixture (PE:EtOAc, 3:1, 50 mL) to give the title compound (8 g, 99.4% yield) as a grey solid.

LCMS: m/z 312.0 (M+H)⁺ (ES⁺).

¹H NMR (DMSO-d₆): δ 8.29 (d, 1H), 7.59 (d, 1H), 7.37 (dd, 1H), 7.23 (s, 1H), 6.53 (br s, 2H).

### Step D: 4-amino-2-fluoro-5-(2-fluoropyridin-4-yl)-3-(prop-1-en-2-yl)benzonitrile

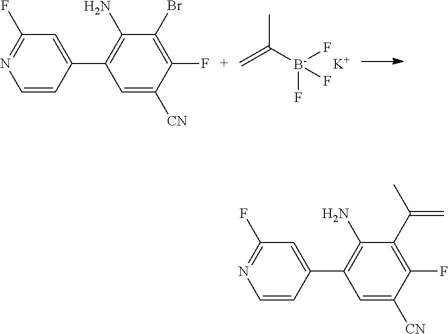

To a solution of 4-amino-3-bromo-2-fluoro-5-(2-fluoropyridin-4-yl)benzonitrile (8 g, 25.8 mmol) in dioxane (150 mL) and H₂O (30 mL) was added potassium trifluoro-(prop-1-en-2-yl)borate (4.96 g, 33.5 mmol), Na₂CO₃ (6.84 g, 64.5 mmol) and Pd(dppf)Cl₂.DCM (1.05 g, 1.29 mmol) under N₂. The mixture was stirred at 100° C. for 3 h. The residue was purified by FC (PE:EtOAc, 1:1) to give the title compound (6.1 g, 87.2% yield) as a yellow solid.

LCMS: m/z 271.9 (M+H)⁺ (ES⁺).

¹H NMR (CDCl₃): δ 8.27 (d, 1H), 7.19-7.17 (m, 2H), 6.94 (s, 1H), 5.50 (s, 1H), 5.09 (s, 1H), 4.53 (br s, 2H), 2.01 (s, 3H).

### Step E: 4-amino-2-fluoro-5-(2-fluoropyridin-4-yl)-3-isopropylbenzonitrile

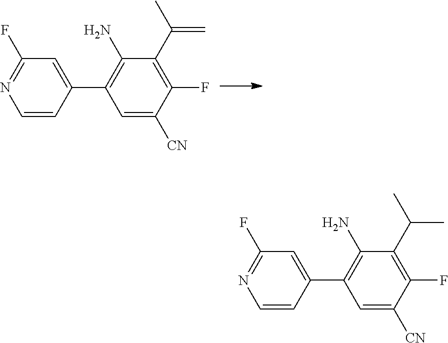 [SMILES: C=C(C)c1c(N)c(-c2ccnc(F)c2)cc(C#N)c1F ; CC(C)c1c(N)c(-c2ccnc(F)c2)cc(C#N)c1F]

To a solution of 4-amino-2-fluoro-5-(2-fluoropyridin-4-yl)-3-(prop-1-en-2-yl)-benzonitrile (4 g, 14.8 mmol) in MeOH (30 mL) and EtOAc (30 mL) was added Rh/Al₂O₃ (30 mg) under N₂. The mixture was stirred at 35° C. for 48 h under H₂ (15 psi). The mixture was filtered and the filtrate was concentrated in vacuum. The residue was purified by FC (PE:EtOAc, 8:1 to 5:1) to give the title compound (2.2 g, 54.6% yield) as a yellow solid.

LCMS: m/z 274.1 (M+H)⁺ (ES⁺).

¹H NMR (CDCl₃): δ 8.34 (d, 1H), 7.24 (dd, 1H), 7.17 (d, 1H), 6.99 (s, 1H), 4.44 (br s, 2H), 3.11-3.04 (m, 1H), 1.43 (dd, 6H).

### Step F: 4-bromo-2-fluoro-5-(2-fluoropyridin-4-yl)-3-isopropylbenzonitrile

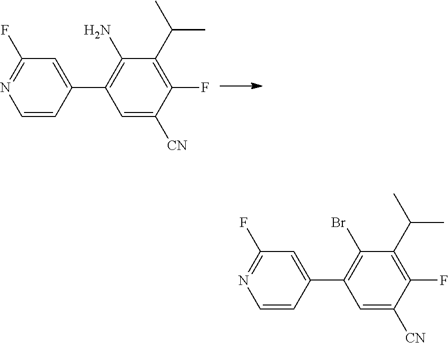 [SMILES: CC(C)c1c(N)c(-c2ccnc(F)c2)cc(C#N)c1F ; CC(C)c1c(F)c(C#N)cc(-c2ccnc(F)c2)c1Br]

To a solution of 4-amino-2-fluoro-5-(2-fluoropyridin-4-yl)-3-isopropylbenzonitrile (2.1 g, 7.68 mmol) in MeCN (40 mL) was added a solution of HBr (7.00 mL, 51.6 mmol, 40% purity in H₂O) in H₂O (4 mL) at 0° C., followed by a solution of NaNO₂ (636 mg, 9.22 mmol) in H₂O (4 mL) at 0° C. The resulting mixture was stirred at 0° C. for 45 min, then CuBr₂ (858 mg, 3.84 mmol) and CuBr (55 mg, 384 μmol) were added and the resulting mixture was stirred at 25° C. for 1 h. The mixture was quenched with H₂O (30 mL) and extracted with EtOAc (20 mL×3). The organic phases were washed with brine (40 mL), dried over anhydrous Na₂SO₄, filtered and concentrated in vacuum. The residue was purified by FC (PE:EtOAc, 33:1 to 26:1) to give the title compound (1.62 g, 62.5% yield) as a white solid.

LCMS: m/z 336.9 (M+H)⁺ (ES⁺).

¹H NMR (CDCl₃): δ 8.33 (d, 1H), 7.38 (dd, 1H), 7.14 (d, 1H), 6.91 (s, 1H), 3.75-3.68 (m, 1H), 1.43 (dd, 6H).

### Step G: tert-butyl 2-(4-cyano-3-fluoro-6-(2-fluoropyridin-4-yl)-2-isopropylphenyl)-acetate

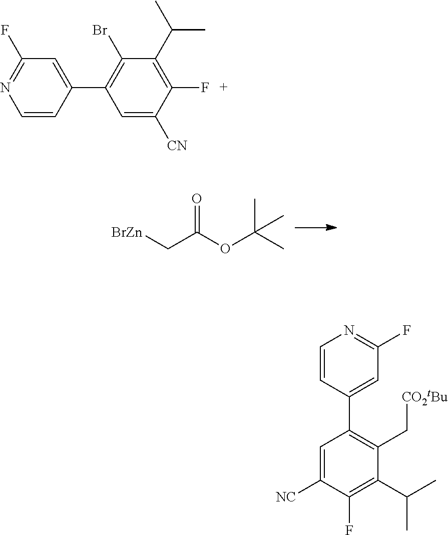

To a solution of 4-bromo-2-fluoro-5-(2-fluoropyridin-4-yl)-3-isopropylbenzonitrile (1.6 g, 4.75 mmol) in THF (10 mL) was added palladium; tri-tert-butylphosphane (121 mg, 237 μmol) and (2-(tert-butoxy)-2-oxoethyl)zinc(II) bromide (0.5 M in THF, 47.5 mL) under N₂. The solution was stirred at 70° C. for 1 h, then the mixture was quenched with sat aq NH₄Cl (30 mL) and extracted with EtOAc (50 mL). The organic phase was washed with brine (30 mL), dried over anhydrous Na₂SO₄, filtered and concentrated in vacuum. The residue was purified by reverse phase flash chromatography (water (0.1% TFA)-MeCN) to give the title compound (0.6 g, 34.0% yield) as a yellow solid.

LCMS: m/z 373.1 (M+H)⁺ (ES⁺).

¹H NMR (CDCl₃): δ 8.30 (d, 1H), 7.34 (dd, 1H), 7.11 (dd, 1H), 6.89 (s, 1H), 3.51 (s, 2H), 3.14-3.09 (m, 1H), 1.48 (s, 9H), 1.41 (dd, 6H).

### Intermediate B13: tert-butyl 2-(4-(difluoromethoxy)-3-fluoro-6-(2-fluoropyridin-4-yl)-2-isopropylphenyl)acetate

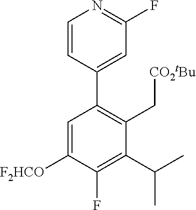 [SMILES: CC(C)c1c(F)c(OC(F)F)cc(-c2ccnc(F)c2)c1CC(=O)OC(C)(C)C]

### Step A: 2-bromo-4-(difluoromethoxy)-5-fluoroaniline

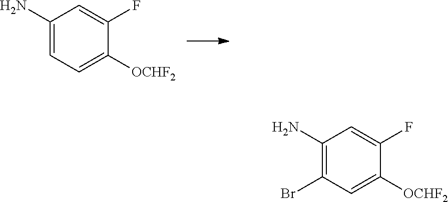 [SMILES: Nc1ccc(OC(F)F)c(F)c1 ; Nc1cc(F)c(OC(F)F)cc1Br]

To a solution of 4-(difluoromethoxy)-3-fluoroaniline (4 g, 22.6 mmol) in MeCN (40 mL) was added NBS (4.22 g, 23.7 mmol). The mixture was stirred at 25° C. for 2 h, then poured into water (100 mL) and extracted with EtOAc (150 mL×3). The organic layers were dried over anhydrous Na₂SO₄, filtered and concentrated in vacuum. The residue was purified by FC (PE:EtOAc, 30:1 to 10:1) to give the title compound (5.5 g, 95.1% yield) as red oil.

LCMS: m/z 258.2 (M+H)⁺ (ES⁺).

¹H NMR (CDCl₃): δ 7.32 (d, 1H), 6.60-6.24 (m, 2H), 4.15 (br s, 2H).

### Step B: 4-(difluoromethoxy)-5-fluoro-2-(2-fluoropyridin-4-yl)aniline

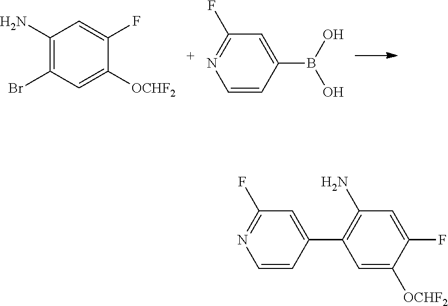

To a solution of 2-bromo-4-(difluoromethoxy)-5-fluoroaniline (5.5 g, 19.5 mmol, 90.8% purity) in dioxane (80 mL) and H₂O (16 mL) was added Pd(dppf)Cl₂.DCM (796 mg, 975 μmol), Na₂CO₃ (4.13 g, 39.0 mmol) and (2-fluoropyridin-4-yl)boronic acid (2.89 g, 20.5 mmol). The mixture was stirred at 90° C. for 12 h under N₂, then poured into water (100 mL) and extracted with EtOAc (200 mL×3). The organic layers were dried over anhydrous Na₂SO₄, filtered and concentrated in vacuum. The residue was purified by FC (PE:EtOAc, 20:1 to 5:1) to give the title compound (5.1 g, 96.1% yield) as a yellow solid.

LCMS: m/z 273.1 (M+H)⁺ (ES⁺).

¹H NMR (CDCl₃): δ 8.32 (d, 1H), 7.29-7.27 (m, 1H), 7.07-6.98 (m, 2H), 6.57 (s, 1H), 6.47 (t, 1H), 3.90 (br s, 2H).

### Step C: 2-bromo-4-(difluoromethoxy)-3-fluoro-6-(2-fluoropyridin-4-yl)aniline

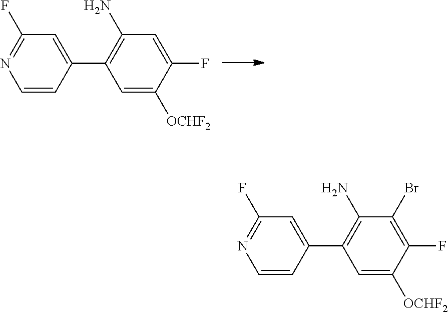 [SMILES: Nc1cc(F)c(OC(F)F)cc1-c1ccnc(F)c1 ; Nc1c(-c2ccnc(F)c2)cc(OC(F)F)c(F)c1Br]

To a solution of 4-(difluoromethoxy)-5-fluoro-2-(2-fluoropyridin-4-yl)aniline (6.1 g, 22.4 mmol) in MeCN (5 mL) was added NBS (4.19 g, 23.5 mmol). The mixture was stirred at 25° C. for 2 h, then concentrated in vacuum. The residue was purified by FC (PE:EtOAc, 10:1 to 5:1) to give the title compound (7.8 g, 99.1% yield) as a yellow solid.

LCMS: m/z 351.1 (M+H)⁺ (ES⁺).

¹H NMR (CDCl₃): δ 8.34 (d, 1H), 7.29-7.27 (m, 1H), 7.05 (s, 1H), 7.03 (s, 1H), 6.49 (t, 1H), 4.38 (br s, 2H).

### Step D: 4-(difluoromethoxy)-3-fluoro-6-(2-fluoropyridin-4-yl)-2-(prop-1-en-2-yl)-aniline

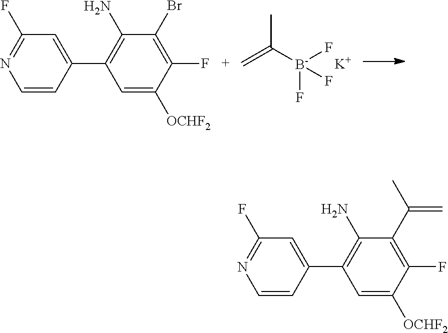

To a solution of 2-bromo-4-(difluoromethoxy)-3-fluoro-6-(2-fluoropyridin-4-yl)aniline (7.5 g, 21.4 mmol) in dioxane (80 mL) and H₂O (16 mL) was added Pd(dppf)Cl₂.DCM (872 mg, 1.07 mmol), Na₂CO₃ (6.79 g, 64.1 mmol) and potassium trifluoro(prop-1-en-2-yl)borate (3.79 g, 25.6 mmol). The mixture was stirred at 100° C. for 12 h under N₂, then poured into water (200 mL) and extracted with EtOAc (200 mL×3). The organic layers were dried over anhydrous Na₂SO₄, filtered and concentrated in vacuum. The residue was purified by FC (PE:EtOAc, 10:1 to 4:1) to give the title compound (6 g, 90.0% yield) as a yellow solid.

LCMS: m/z 313.3 (M+H)⁺ (ES⁺).

¹H NMR (CDCl₃): δ 8.31 (d, 1H), 7.31 (d, 1H), 7.05 (s, 1H), 6.96 (d, 1H), 6.48 (t, 1H), 5.54 (t, 1H), 5.15 (s, 1H), 4.04 (br s, 2H), 2.09 (s, 3H).

### Step E: 4-(difluoromethoxy)-3-fluoro-6-(2-fluoropyridin-4-yl)-2-isopropylaniline

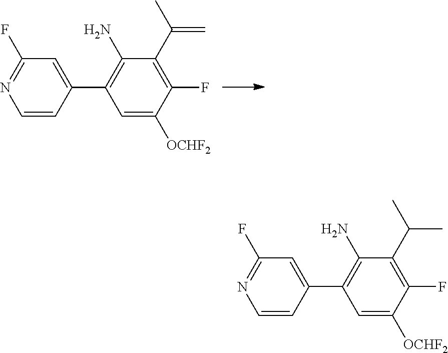 [SMILES: C=C(C)c1c(N)c(-c2ccnc(F)c2)cc(OC(F)F)c1F ; CC(C)c1c(N)c(-c2ccnc(F)c2)cc(OC(F)F)c1F]

To a solution of 4-(difluoromethoxy)-3-fluoro-6-(2-fluoropyridin-4-yl)-2-(prop-1-en-2-yl)aniline (6 g, 19.2 mmol) in MeOH (200 mL) was added Pd/C (600 mg, 10% loading on active carbon) under N₂. The suspension was degassed in vacuum and purged with H₂ several times. The mixture was stirred at 25° C. for 12 h under H₂ (15 psi). The reaction mixture was filtered and the filtrate was concentrated in vacuum to give the title compound (5.8 g, 96.1% yield) as a yellow oil.

LCMS: m/z 315.1 (M+H)⁺ (ES⁺).

¹H NMR (CDCl₃): δ 8.31 (d, 1H), 7.27-7.26 (m, 1H), 7.02 (d, 1H), 6.88 (d, 1H), 6.46 (t, 1H), 3.86 (br s, 2H), 3.15-3.09 (m, 1H), 1.41 (dd, 6H).

### Step F: 4-(2-bromo-5-(difluoromethoxy)-4-fluoro-3-isopropylphenyl)-2-fluoropyridine

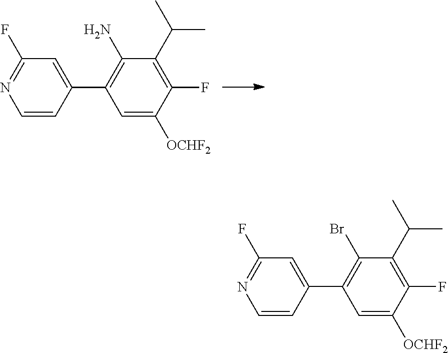 [SMILES: CC(C)c1c(N)c(-c2ccnc(F)c2)cc(OC(F)F)c1F ; CC(C)c1c(F)c(OC(F)F)cc(-c2ccnc(F)c2)c1Br]

To a solution of 4-(difluoromethoxy)-3-fluoro-6-(2-fluoropyridin-4-yl)-2-isopropyl-aniline (5.8 g, 18.5 mmol) in MeCN (100 mL) was added CuBr₂ (4.95 g, 22.2 mmol), CuBr (265 mg, 1.85 mmol) and isopentyl nitrite (3.24 g, 27.7 mmol). The mixture was stirred at 50° C. for 0.5 h, then quenched with 1N aq HCl (50 mL) and extracted with EtOAc (100 mL×3). The organic layers were dried over anhydrous Na₂SO₄, filtered and concentrated in vacuum. The residue was purified by FC (PE:EtOAc, 10:1 to 4:1) to give the title compound (6.5 g, 92.0% yield, 98.8% purity on LCMS) as colourless oil.

LCMS: m/z 378.0 (M+H)⁺ (ES⁺).

¹H NMR (CDCl₃): δ 8.29 (d, 1H), 7.18-7.16 (m, 1H), 7.05 (d, 1H), 6.93 (s, 1H), 6.57 (t, 1H), 3.74-3.64 (m, 1H), 1.41 (dd, 6H).

### Step G: tert-butyl 2-(4-(difluoromethoxy)-3-fluoro-6-(2-fluoropyridin-4-yl)-2-isopropylphenyl)acetate

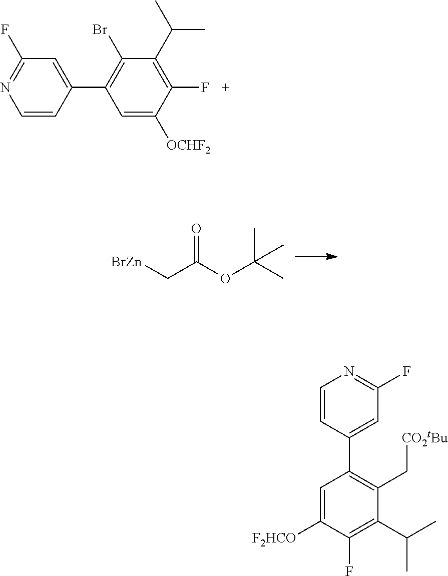

To a solution of 4-(2-bromo-5-(difluoromethoxy)-4-fluoro-3-isopropylphenyl)-2-fluoropyridine (6 g, 15.9 mmol), Pd₂(dba)₃ (726 mg, 793 μmol) and XPhos (756 mg, 1.59 mmol) in THF (60 mL) was added (2-(tert-butoxy)-2-oxoethyl)zinc(II) bromide (0.51 M in THF, 93.3 mL). The mixture was stirred at 70° C. for 2 h under N₂, then quenched with sat aq NH₄Cl (200 mL) and extracted with EtOAc (200 mL×3). The organic layers were dried over anhydrous Na₂SO₄, filtered and concentrated in vacuum. The residue was purified by reverse phase flash chromatography (0.1% TFA) to give the title compound (6 g, 91.47% yield) as a yellow solid.

LCMS: m/z 414.1 (M+H)⁺ (ES⁺).

¹H NMR (CDCl₃): δ 8.27 (d, 1H), 7.15-7.13 (m, 1H), 6.97 (d, 1H), 6.91 (s, 1H), 6.56 (t, 1H), 3.44 (s, 2H), 3.13-3.03 (m, 1H), 1.46 (s, 9H), 1.39 (d, 6H).

### Intermediate B14: 2-(4-fluoro-2-(2-fluoropyridin-4-yl)-6-isopropylphenyl)acetic acid

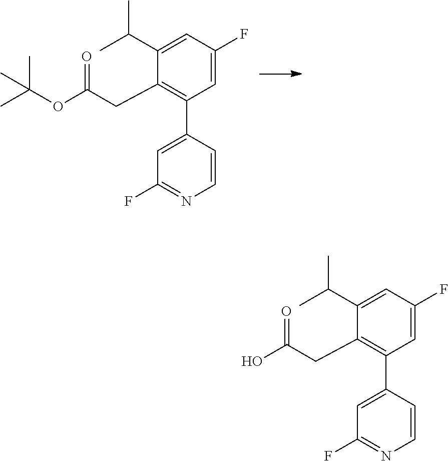 [SMILES: CC(C)c1cc(F)cc(-c2ccnc(F)c2)c1CC(=O)OC(C)(C)C ; CC(C)c1cc(F)cc(-c2ccnc(F)c2)c1CC(=O)O]

To a solution of tert-butyl 2-(4-fluoro-2-(2-fluoropyridin-4-yl)-6-isopropylphenyl)-acetate (Intermediate B4) (4.0 g, 11.5 mmol) in DCM (20 mL) was added TFA (270 mmol, 20 mL). The reaction solution was stirred at 20° C. for 12 h, then concentrated in vacuum to give the title compound (4.0 g, crude, TFA salt) as a red solid.

LCMS: m/z 292.1 (M-TFA+H)⁺ (ES⁺).

### Intermediate B1s: tert-butyl 2-(2-(2-fluoropyridin-4-yl)-6-isopropyl-4-(methoxy-methyl)phenyl)acetate

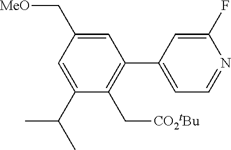 [SMILES: COCc1cc(-c2ccnc(F)c2)c(CC(=O)OC(C)(C)C)c(C(C)C)c1]

### Step A: methyl 4-amino-3-(prop-1-en-2-yl)benzoate

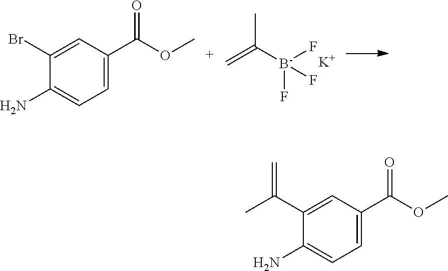

To a solution of methyl 4-amino-3-bromobenzoate (4.0 g, 17.4 mmol) in dioxane (80 mL) and H₂O (16 mL) was added potassium trifluoro(prop-1-en-2-yl)borate (5.15 g, 34.8 mmol), K₂CO₃ (7.21 g, 52.2 mmol) and Pd(dppf)Cl₂.DCM (1.42 g, 1.74 mmol). The reaction mixture was stirred at 95° C. for 3 h under N₂. Water (100 mL) and EtOAc (100 mL) were added into the reaction mixture and the mixture was separated. The aqueous layer was extracted with EtOAc (100 mL×2). The combined organic phases were dried over anhydrous Na₂SO₄, filtered and concentrated under reduced pressure. The residue was purified by FC (PE:EtOAc, 1:0 to 10:1) to give the title compound (2.8 g, 83.4% yield, 99.0% purity on LCMS) as a yellow oil.

LCMS: m/z 192.2 (M+H)⁺ (ES⁺).

¹H NMR (CDCl₃): δ 7.76-7.74 (m, 2H), 6.66 (dd, 1H), 5.34 (dd, 1H), 5.08 (d, 1H), 4.27 (br s, 2H), 3.86 (s, 3H), 2.08 (s, 3H).

### Step B: methyl 4-amino-3-isopropylbenzoate

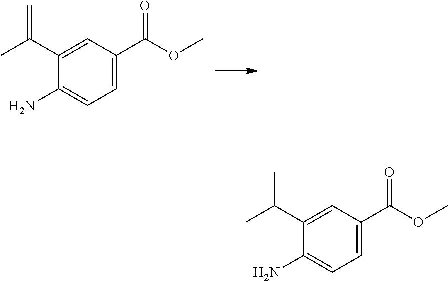

To a solution of methyl 4-amino-3-(prop-1-en-2-yl)benzoate (6.5 g, 34.0 mmol) in MeOH (120 mL) was added Pd/C (600 mg, 10% purity loaded on active carbon) under N₂. The mixture was degassed in vacuum and purged with H₂ several times. The mixture was stirred at 20° C. for 12 h under H₂ (15 psi). The reaction mixture was filtered and the filtrate was concentrated under reduced pressure. The residue was purified by FC (PE:EtOAc, 1:0 to 2:1) to give the title compound (4.7 g, 70.7% yield, 98.8% purity on LCMS) as a white solid.

LCMS: m/z 194.2 (M+H)⁺ (ES⁺).

¹H NMR (CDCl₃): δ 7.85 (d, 1H), 7.73 (dd, 1H), 6.64 (d, 1H), 4.09 (br s, 2H), 3.87 (s, 3H), 2.91-2.79 (m, 1H), 1.29 (d, 6H).

### Step C: methyl 4-amino-3-bromo-5-isopropylbenzoate

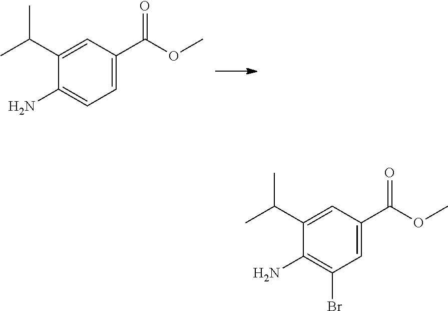

To a solution of methyl 4-amino-3-isopropylbenzoate (4.7 g, 24.3 mmol) in MeCN (80 mL) was added NBS (4-33 g, 24.3 mmol) in portions at 0° C. The reaction mixture was stirred at 20° C. for 2 h. Water (50 mL) and EtOAc (50 mL) were added into the reaction mixture and the mixture was separated. The aqueous layer was extracted with EtOAc (50 mL×2). The combined organic phases were dried over anhydrous Na₂SO₄, filtered and concentrated under reduced pressure. The residue was purified by FC (PE:EtOAc, 1:0 to 10:1) to give the title compound (6.2 g, 92.6% yield, 98.9% purity on LCMS) as a yellow solid.

LCMS: m/z 272.0 (M+H)⁺ (ES⁺).

¹H NMR (CDCl₃): δ 8.03 (d, 1H), 7.78 (d, 1H), 4.60 (br s, 2H), 3.87 (s, 3H), 2.91-2.82 (m, 1H) and 1.30 (d, 6H).

### Step D: methyl 4-amino-3-(2-fluoropyridin-4-yl)-5-isopropylbenzoate

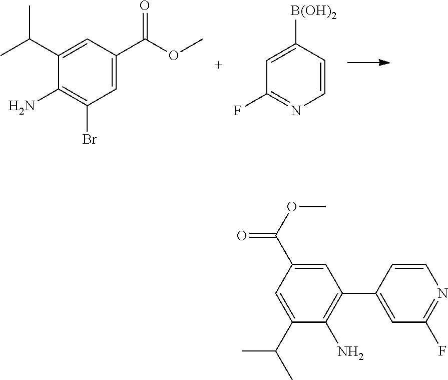 [SMILES: COC(=O)c1cc(Br)c(N)c(C(C)C)c1 ; OB(O)c1ccnc(F)c1 ; COC(=O)c1cc(-c2ccnc(F)c2)c(N)c(C(C)C)c1]

To a solution of methyl 4-amino-3-bromo-5-isopropylbenzoate (5-7 g, 21.0 mmol) in dioxane (110 mL) and H₂O (22 mL) was added (2-fluoropyridin-4-yl)boronic acid (3.54 g, 25.1 mmol), Pd(dppf)Cl₂.DCM (1.71 g, 2.09 mmol) and K₂CO₃ (8.68 g, 62.8 mmol). After the addition, the reaction mixture was stirred at 100° C. for 3 h under N₂. Water (100 mL) and EtOAc (100 mL) were added into the reaction mixture and the mixture was separated. The aqueous layer was extracted with EtOAc (100 mL×2). The combined organic phases were dried over anhydrous Na₂SO₄, filtered and concentrated under reduced pressure. The residue was purified by FC (PE:EtOAc, 1:0 to 3:1) to give the title compound (5-7 g, 91.6% yield, 97.0% purity on LCMS) as a yellow solid.

LCMS: m/z 289.1 (M+H)⁺ (ES⁺).

¹H NMR (CDCl₃): δ 8.31 (d, 1H), 7.91 (d, 1H), 7.69 (d, 1H), 7.34-7.30 (m, 1H), 7.06 (s, 1H), 4.26 (br s, 2H), 3.88 (s, 3H), 2.93-2.86 (m, 1H), 1.34 (d, 6H).

### Step E: methyl 4-bromo-3-(2-fluoropyridin-4-yl)-5-isopropylbenzoate

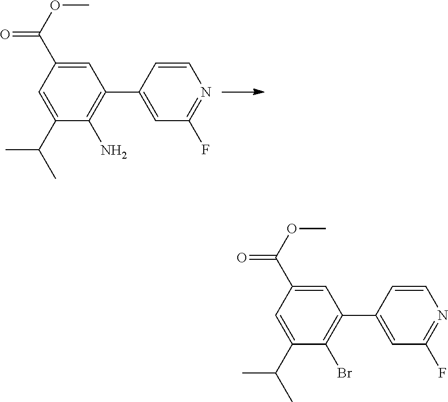 [SMILES: COC(=O)c1cc(-c2ccnc(F)c2)c(N)c(C(C)C)c1 ; COC(=O)c1cc(-c2ccnc(F)c2)c(Br)c(C(C)C)c1]

To a solution of methyl 4-amino-3-(2-fluoropyridin-4-yl)-5-isopropylbenzoate (4.7 g, 16.3 mmol) in MeCN (20 mL) was added CuBr (2.81 g, 19.6 mmol), CuBr₂ (364 mg, 1.63 mmol) and isopentyl nitrite (2.86 g, 24.5 mmol) at 25° C. After the addition, the reaction mixture was stirred at 50° C. for 0.5 h. Water (200 mL) and EtOAc (200 mL) were added into the reaction mixture and the mixture was separated. The aqueous layer was extracted with EtOAc (200 mL×2). The combined organic phases were dried over anhydrous Na₂SO₄, filtered and concentrated under reduced pressure. The residue was purified by FC (PE:EtOAc, 1:0 to 5:1) to give the title compound (4.3 g, 73.3% yield, 97.9% purity on LCMS).

LCMS: m/z 351.9 (M+H)⁺ (ES⁺).

¹H NMR (CDCl₃): δ 8.30 (d, 1H), 8.02 (d, 1H), 7.76 (d, 1H), 7.22-7.20 (m, 1H), 6.97 (t, 1H), 3.94 (s, 3H), 3.59-3.49 (m, 1H), 1.33 (d, 6H).

### Step F: (4-bromo-3-(2-fluoropyridin-4-yl)-5-isopropylphenyl)methanol

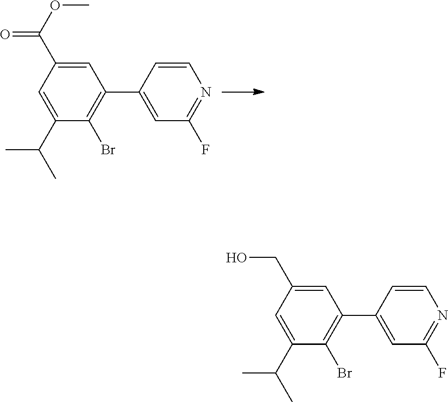 [SMILES: COC(=O)c1cc(-c2ccnc(F)c2)c(Br)c(C(C)C)c1 ; CC(C)c1cc(CO)cc(-c2ccnc(F)c2)c1Br]

To a solution of methyl 4-bromo-3-(2-fluoropyridin-4-yl)-5-isopropylbenzoate (4.6 g, 13.1 mmol) in MeOH (100 mL) was added NaBH₄ (4.94 g, 131 mmol) at 0° C. The reaction mixture was stirred at 60° C. for 24 h, then cooled to RT, slowly poured into cold sat aq NH₄Cl (500 mL), and extracted with EtOAc (200 mL×2). The combined organic phases were dried over anhydrous Na₂SO₄, filtered and the filtrate was concentrated under reduced pressure. The residue was purified by FC (PE:EtOAc, 1:0 to 5:1) to give the title compound (3.6 g, 82.8% yield, 97.4% purity on LCMS) as a white solid.

LCMS: m/z 323.9 (M+H)⁺ (ES⁺).

¹H NMR (CDCl₃): δ 8.26 (d, 1H), 7.37 (d, 1H), 7.21 (dd, 1H), 7.12 (d, 1H), 6.95 (t, 1H), 4.72 (s, 2H), 3.57-3.47 (m, 1H), 1.30 (d, 6H). One exchangeable proton not observed.

### Step G: 4-(2-bromo-3-isopropyl-5-(methoxymethyl)phenyl)-2-fluoropyridine

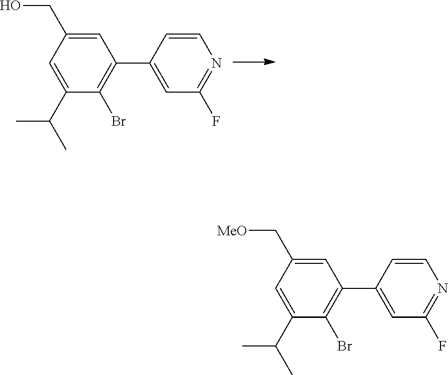 [SMILES: CC(C)c1cc(CO)cc(-c2ccnc(F)c2)c1Br ; COCc1cc(-c2ccnc(F)c2)c(Br)c(C(C)C)c1]

To a solution of (4-bromo-3-(2-fluoropyridin-4-yl)-5-isopropylphenyl)methanol (2.5 g, 7.71 mmol) in DMF (25 mL) was added Ag₂O (1.79 g, 7.71 mmol) and MeI (1.44 mL, 23.1 mmol) at 0° C. The mixture was stirred at 25° C. for 12 h, then poured into water (30 mL) and extracted with EtOAc (40 mL×3). The organic layers were washed with sat aq NaCl (20 mL×3), dried over anhydrous Na₂SO₄, filtered and concentrated in vacuum. The residue was purified by FC (PE:EtOAc, 30:1 to 10:1) to give the title compound (2 g, 76.7% yield) as a yellow oil.

LCMS: m/z 338.0 (M+H)⁺ (ES⁺).

¹H NMR (CDCl₃): δ 8.26 (d, 1H), 7.32 (d, 1H), 7.21 (d, 1H), 7.08 (d, 1H), 6.96 (s, 1H), 4.45 (s, 2H), 3.54-3.42 (m, 4H), 1.30 (d, 6H).

### Step H: tert-butyl 2-(2-(2-fluoropyridin-4-yl)-6-isopropyl-4-(methoxymethyl)phenyl)-acetate

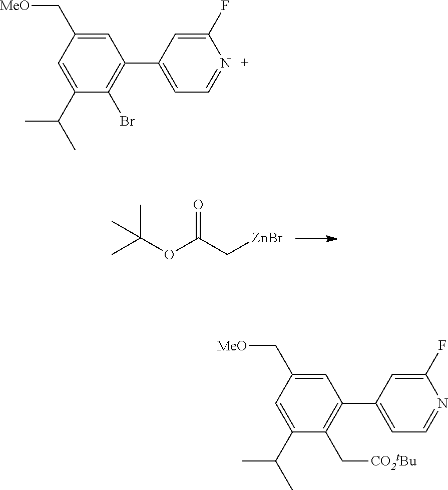

To a solution of 4-(2-bromo-3-isopropyl-5-(methoxymethyl)phenyl)-2-fluoropyridine (2 g, 5.91 mmol), Pd₂(dba)₃ (271 mg, 296 μmol) and XPhos (282 mg, 591 μmol) in THF (20 mL) was added (2-(tert-butoxy)-2-oxoethyl)zinc(II) bromide (0.5 M in THF, 34.8 mL). The mixture was stirred at 70° C. for 12 h under N₂, then quenched with sat aq NH₄Cl (50 mL) and extracted with EtOAc (50 mL×3). The organic layers were dried over anhydrous Na₂SO₄, filtered and concentrated in vacuum. The residue was purified by FC (PE:EtOAc, 30:1 to 15:1) to give the title compound (1.4 g, 63.4% yield, 100% purity on LCMS) as a yellow oil.

LCMS: m/z 374.4 (M+H)⁺ (ES⁺).

¹H NMR (CDCl₃): δ 8.23 (d, 1H), 7.36 (d, 1H), 7.18 (d, 1H), 7.03 (d, 1H), 6.94 (s, 1H), 4.46 (s, 2H), 3.48 (s, 2H), 3.43 (s, 3H), 3.13-3.06 (m, 1H), 1.44 (s, 9H), 1.27 (d, 6H).

### Intermediate B16: tert-butyl 2-(3-fluoro-6-(2-fluoropyridin-4-yl)-2-isopropyl-4-(methoxymethyl)phenyl)acetate

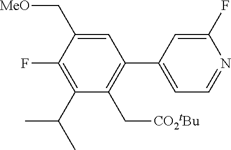 [SMILES: COCc1cc(-c2ccnc(F)c2)c(CC(=O)OC(C)(C)C)c(C(C)C)c1F]

### Step A: methyl 4-amino-5-bromo-2-fluorobenzoate

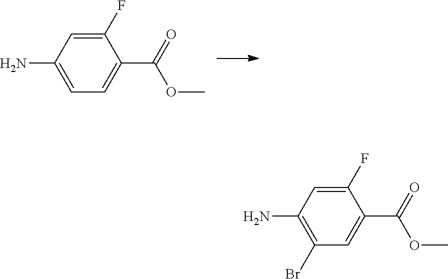

To a solution of methyl 4-amino-2-fluorobenzoate (24.5 g, 145 mmol) in MeCN (250 mL) was added dropwise a solution of NBS (23.2 g, 130 mmol) in MeCN (250 mL) at 0° C. The mixture was stirred at 25° C. for 12 h, then diluted with H₂O (500 mL) and extracted with EtOAc (500 mL×3). The combined organic phases were washed with brine (500 mL), dried over anhydrous Na₂SO₄, filtered and concentrated in vacuum. The residue was purified by FC (PE:EtOAc, 100:1 to 10:1) to give the title compound (12.5 g, 34.8% yield) as a yellow oil.

LCMS: m/z 250.0 (M+H)⁺ (ES⁺).

¹H NMR (CDCl₃): δ 8.04 (d, 1H), 6.46 (d, 1H), 4.60 (br s, 2H) and 3.88 (s, 3H).

### Step B: methyl 4-amino-2-fluoro-5-(2-fluoropyridin-4-yl)benzoate

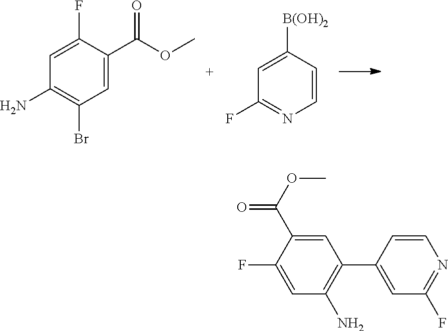 [SMILES: COC(=O)c1cc(Br)c(N)cc1F ; OB(O)c1ccnc(F)c1 ; COC(=O)c1cc(-c2ccnc(F)c2)c(N)cc1F]

To a solution of methyl 4-amino-5-bromo-2-fluorobenzoate (12 g, 48.4 mmol) and (2-fluoropyridin-4-yl)boronic acid (7.50 g, 53.2 mmol) in dioxane (200 mL) and H₂O (50 mL) were added Na₂CO₃ (12.8 g, 121 mmol) and Pd(dppf)Cl₂.DCM (3.95 g, 4.84 mmol) under N₂. The mixture was stirred at 95° C. for 12 h, then diluted with H₂O (300 mL) and extracted with EtOAc (300 mL×3). The combined organic phases were washed with brine (300 mL), dried over anhydrous Na₂SO₄, filtered and concentrated in vacuum. The residue was purified by FC (PE:EtOAc, 3:1 to 0:1) to give the title compound (10 g, 78.23% yield) as a yellow solid.

LCMS: m/z 264.9 (M+H)⁺ (ES⁺).

¹H NMR (CDCl₃): δ 8.29 (d, 1H), 7.74 (d, 1H), 7.27-7.25 (m, 1H), 7.01 (s, 1H), 6.46 (d, 1H), 4.32 (br s, 2H), 3.88 (s, 3H).

### Step C: methyl 4-amino-3-bromo-2-fluoro-5-(2-fluoropyridin-4-yl)benzoate

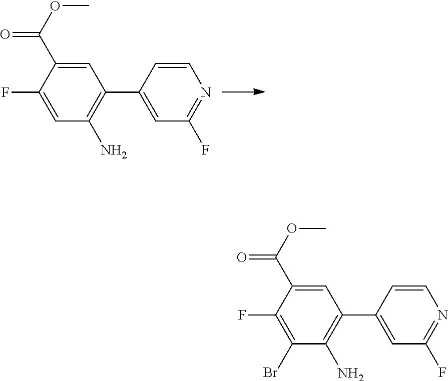 [SMILES: COC(=O)c1cc(-c2ccnc(F)c2)c(N)cc1F ; COC(=O)c1cc(-c2ccnc(F)c2)c(N)c(Br)c1F]

To a solution of methyl 4-amino-2-fluoro-5-(2-fluoropyridin-4-yl)benzoate (10 g, 37.9 mmol) in DCM (200 mL) and DMF (50 mL) was added NBS (8.08 g, 45.4 mmol) at 0° C. The mixture was stirred at 25° C. for 3 h, then washed with H₂O (200 mL) and extracted with EtOAc (200 mL×3). The combined organic phases were washed with brine (200 mL), dried over anhydrous Na₂SO₄, filtered and the filtrate was concentrated in vacuum. The crude product was triturated with EtOAc (50 mL) to give the title compound (9 g, 69.3% yield) as a yellow solid.

LCMS: m/z 344.8 (M+H)⁺ (ES⁺).

¹H NMR (CDCl₃): δ 8.33 (d, 1H), 7.72 (d, 1H), 7.28-7.25 (m, 1H), 7.01 (s, 1H), 4.82 (br s, 2H), 3.89 (s, 3H).

### Step D: methyl 4-amino-2-fluoro-5-(2-fluoropyridin-4-yl)-3-(prop-1-en-2-yl)benzoate

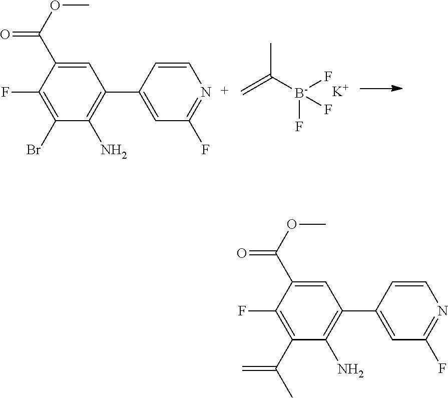

To a solution of methyl 4-amino-3-bromo-2-fluoro-5-(2-fluoropyridin-4-yl)benzoate (8 g, 23.3 mmol) and potassium trifluoro(prop-1-en-2-yl)borate (5.18 g, 35.0 mmol) in dioxane (200 mL) and H₂O (40 mL) were added Pd(dppf)Cl₂.DCM (1.90 g, 2.33 mmol) and Na₂CO₃ (6.18 g, 58.3 mmol). The reaction mixture was stirred at 95° C. for 12 h under N₂, then washed with sat aq NaHCO₃ (300 mL) and extracted with EtOAc (300 mL×3). The combined organic phases were washed with brine (300 mL), dried over anhydrous Na₂SO₄, filtered and concentrated in vacuum. The residue was purified by FC (PE:EtOAc, 3:1 to 0:1) to give the title compound (6.5 g, 91.6% yield) as a yellow solid.

LCMS: m/z 305.1 (M+H)⁺ (ES⁺).

¹H NMR (CDCl₃): δ 8.24 (d, 1H), 7.61 (d, 1H), 7.22 (dd, 1H), 6.98 (s, 1H), 5.46 (d, 1H), 5.05 (s, 1H), 4.42 (br s, 2H), 3.81 (s, 3H), 2.00 (s, 3H).

### Step E: methyl 4-amino-2-fluoro-5-(2-fluoropyridin-4-yl)-3-isopropylbenzoate

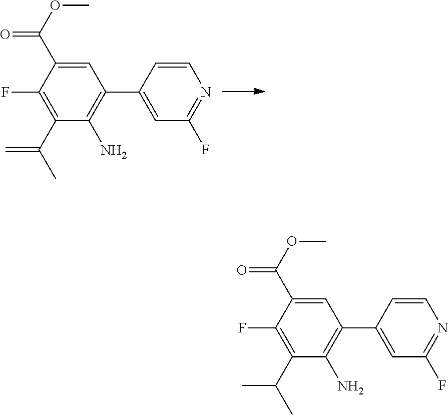 [SMILES: C=C(C)c1c(N)c(-c2ccnc(F)c2)cc(C(=O)OC)c1F ; COC(=O)c1cc(-c2ccnc(F)c2)c(N)c(C(C)C)c1F]

To a solution of methyl 4-amino-2-fluoro-5-(2-fluoropyridin-4-yl)-3-(prop-1-en-2-yl)-benzoate (5.7 g, 18.7 mmol) in MeOH (100 mL) and EtOAc (100 mL) was added Pd/C (0.6 g, 10% purity loaded on active carbon) under N₂. The suspension was degassed under vacuum and purged with H₂ three times. The mixture was stirred at 25° C. for 12 h under H₂ (15 psi), then diluted with EtOAc (200 mL) and filtered through Celite. The filtrate was diluted with H₂O (300 mL) and extracted with EtOAc (300 mL×3). The combined organic phases were washed with brine (300 mL), dried over anhydrous Na₂SO₄, filtered and concentrated in vacuum. The residue was purified by FC (PE:EtOAc, 4:1 to 3:1) to give the title compound (5.74 g, 100% yield) as a white solid.

LCMS: m/z 307.1 (M+H)⁺ (ES⁺).

¹H NMR (CDCl₃): δ 8.31 (d, 1H), 7.59 (d, 1H), 7.28 (s, 1H), 7.02 (s, 1H), 4.33 (br s, 2H), 3.88 (s, 3H) 3.11-3.07 (m, 1H), 1.32 (d, 6H).

### Step F: methyl 4-bromo-2-fluoro-5-(2-fluoropyridin-4-yl)-3-isopropylbenzoate

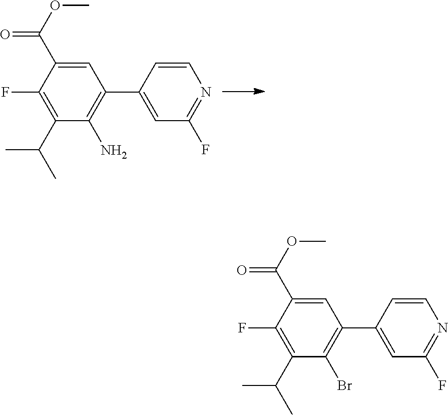 [SMILES: COC(=O)c1cc(-c2ccnc(F)c2)c(N)c(C(C)C)c1F ; COC(=O)c1cc(-c2ccnc(F)c2)c(Br)c(C(C)C)c1F]

To a solution of methyl 4-amino-2-fluoro-5-(2-fluoropyridin-4-yl)-3-isopropylbenzoate (5 g, 16.3 mmol) in MeCN (50 mL) was added isopentyl nitrite (2.29 g, 19.6 mmol) at 0° C., followed by CuBr (2.81 g, 19.6 mmol) at 0° C. The resulting mixture was stirred at 60° C. for 3 h, then diluted with H₂O (100 mL) and extracted with EtOAc (100 mL×3). The combined organic phases were washed with brine (100 mL), dried over anhydrous Na₂SO₄, filtered and concentrated in vacuum. The residue was purified by FC (PE:EtOAc, 5:1 to 4:1) to give the title compound (3.3 g, 54.6% yield) as a white solid.

LCMS: m/z 371.9 (M+H)⁺ (ES⁺).

¹H NMR (CDCl₃): δ 8.30 (d, 1H), 7.68 (d, 1H), 7.19 (dd, 1H), 6.94 (s, 1H), 3.94 (s, 3H), 3.75-3.70 (m, 1H), 1.26 (d, 6H).

### Step G: methyl 4-(2-(tert-butoxy)-2-oxoethyl)-2-fluoro-5-(2-fluoropyridin-4-yl)-3-isopropylbenzoate

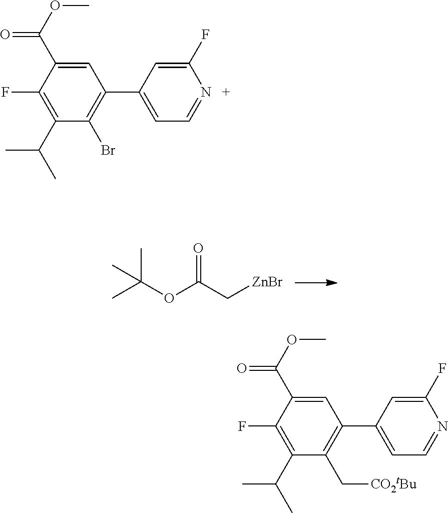

To a solution of methyl 4-bromo-2-fluoro-5-(2-fluoropyridin-4-yl)-3-isopropylbenzoate (2.7 g, 7.29 mmol) in THF (30 mL) was added palladium; tri-tert-butylphosphane (373 mg, 729 μmol) under N₂. Then (2-(tert-butoxy)-2-oxoethyl)zinc(II) bromide (0.5 M in THF, 43.76 mL) was added and the reaction mixture was stirred at 70° C. for 20 min. The reaction mixture was quenched with sat aq NaHCO₃ (50 mL) and extracted with EtOAc (50 mL×3). The combined organic phases were washed with brine (50 mL), dried over anhydrous Na₂SO₄, filtered and concentrated in vacuum. The residue was purified by FC (PE:EtOAc, 90:1 to 80:1) to give the title compound (2.8 g, 94.7% yield) as a yellow oil.

LCMS: m/z 406.2 (M+H)⁺ (ES⁺).

¹H NMR (CDCl₃): δ 8.26 (d, 1H), 7.65 (d, 1H), 7.15 (d, 1H), 6.92 (s, 1H), 3.93 (s, 3H), 3.50 (s, 2H), 3.15-3.08 (m, 1H), 1.45 (s, 9H), 1.40 (d, 6H).

### Step H: tert-butyl 2-(3-fluoro-6-(2-fluoropyridin-4-yl)-4-(hydroxymethyl)-2-isopropyl-phenyl)acetate

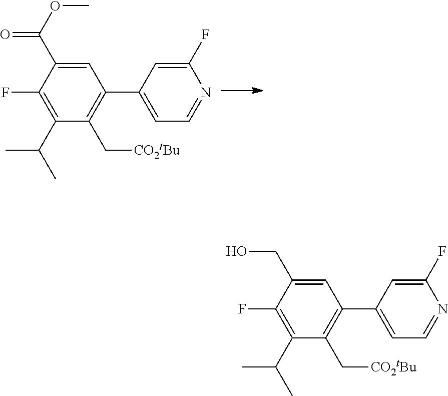 [SMILES: COC(=O)c1cc(-c2ccnc(F)c2)c(CC(=O)OC(C)(C)C)c(C(C)C)c1F ; CC(C)c1c(F)c(CO)cc(-c2ccnc(F)c2)c1CC(=O)OC(C)(C)C]

To a mixture of methyl 4-(2-(tert-butoxy)-2-oxoethyl)-2-fluoro-5-(2-fluoropyridin-4-yl)-3-isopropylbenzoate (2.3 g, 5.67 mmol) in MeOH (30 mL) was added NaBH₄ (1.07 g, 28.4 mmol) at 25° C. under N₂. The reaction mixture was stirred at 60° C. for 12 h, then quenched with 0.5 M aq HCl (40 mL), and extracted with EtOAc (40 mL×3). The combined organic phases were washed with brine (40 mL, dried over anhydrous Na₂SO₄, filtered and concentrated in vacuum. The residue was purified by FC (PE:EtOAc, 10:1 to 5:1) to give the title compound (0.5 g, 23.4% yield) as a yellow oil.

LCMS: m/z 378.0 (M+H)⁺ (ES⁺).

¹H NMR (CDCl₃): δ 8.25 (d, 1H), 7.18-7.14 (m, 2H), 6.91 (s, 1H), 4.79 (s, 2H), 3.47 (s, 2H), 3.11-3.05 (m, 1H), 1.45 (s, 9H), 1.38 (s, 6H). One exchangeable proton not observed.

### Step I: tert-butyl 2-(3-fluoro-6-(2-fluoropyridin-4-yl)-2-isopropyl-4-(methoxymethyl)-phenyl)acetate

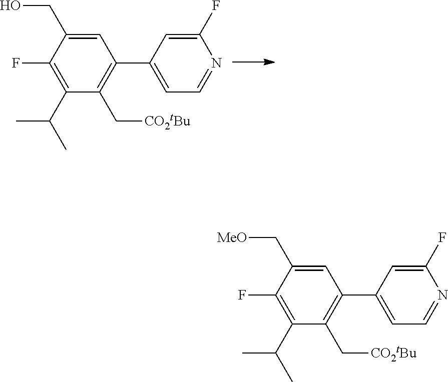 [SMILES: CC(C)c1c(F)c(CO)cc(-c2ccnc(F)c2)c1CC(=O)OC(C)(C)C ; COCc1cc(-c2ccnc(F)c2)c(CC(=O)OC(C)(C)C)c(C(C)C)c1F]

To a solution of tert-butyl 2-(3-fluoro-6-(2-fluoropyridin-4-yl)-4-(hydroxymethyl)-2-isopropylphenyl)acetate (0.55 g, 1-46 mmol) in THF (10 mL) was added NaH (117 mg, 2.91 mmol, 60% purity in mineral oil) at 0° C. The mixture was stirred at 25° C. for 20 min, then methyl iodide (414 mg, 2.91 mmol) was added at 25° C. The reaction mixture was stirred at 25° C. for 3 h, then quenched with H₂O (20 mL) and extracted with EtOAc (20 mL×3). The combined organic phases were washed with brine (20 mL), dried over anhydrous Na₂SO₄, filtered and concentrated in vacuum. The residue was purified by FC (PE:EtOAc, 20:1 to 10:1) to give the title compound (0.48 g, 84.2% yield) as a yellow solid.

LCMS: m/z 392.2 (M+H)⁺ (ES⁺).

¹H NMR (CDCl₃): δ 8.25 (d, 1H), 7.17-7.11 (m, 2H), 6.92 (s, 1H), 4.55 (s, 2H), 3.50 (s, 2H), 3.48 (s, 3H), 3.10-3.04 (m, 1H), 1.45 (s, 9H), 1.37 (d, 6H).

### Intermediate B17: methyl 2-(4-fluoro-2-isopropyl-6-(2-(((trifluoromethyl)sulfonyl)-oxy)pyridin-4-yl)phenyl)acetate

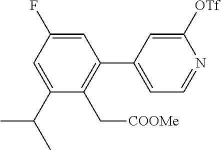 [SMILES: COC(=O)Cc1c(-c2ccnc(OS(=O)(=O)C(F)(F)F)c2)cc(F)cc1C(C)C]

### Step A: 2-(4-fluoro-2-(2-hydroxypyridin-4-yl)-6-isopropylphenyl)acetic acid

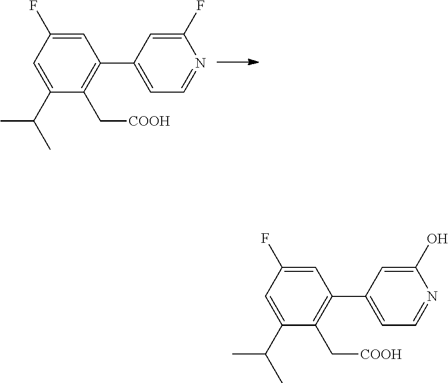 [SMILES: CC(C)c1cc(F)cc(-c2ccnc(F)c2)c1CC(=O)O ; CC(C)c1cc(F)cc(-c2ccnc(O)c2)c1CC(=O)O]

To a solution of 2-(4-fluoro-2-(2-fluoropyridin-4-yl)-6-isopropylphenyl)acetic acid (Intermediate B14) (4.5 g, 11.1 mmol, TFA salt) in H₂O (100 mL) was added NaOH (8.88 g, 222 mmol). The mixture was stirred at 80° C. for 24 h, adjusted to pH 7 with 1 M aq HCl, then concentrated in vacuum. The residue was purified by reverse phase flash chromatography (water (0.1% TFA)-MeCN) to give the title compound (1.9 g, 59.2% yield) as a light yellow solid.

LCMS: m/z 290.1 (M+H)⁺ (ES⁺).

¹H NMR (DMSO-d₆): δ 7.43 (d, 1H), 7.21 (dd, 1H), 6.89 (dd, 1H), 6.16 (s, 1H), 6.07 (dd, 1H), 3.54 (s, 2H), 3.03-2.99 (m, 1H), 1.18 (d, 6H). Two exchangeable protons not observed.

### Step B: methyl 2-(4-fluoro-2-(2-hydroxypyridin-4-yl)-6-isopropylphenyl)acetate

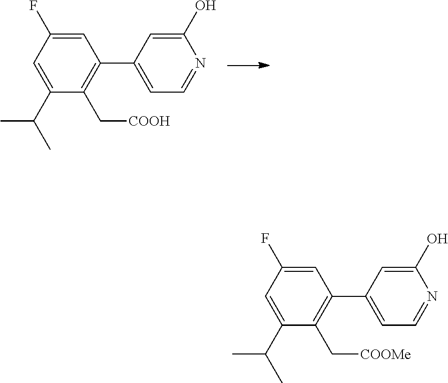 [SMILES: CC(C)c1cc(F)cc(-c2ccnc(O)c2)c1CC(=O)O ; COC(=O)Cc1c(-c2ccnc(O)c2)cc(F)cc1C(C)C]

A solution of 2-(4-fluoro-2-(2-hydroxypyridin-4-yl)-6-isopropylphenyl)acetic acid (1.9 g, 6.57 mmol) and conc H₂SO₄ (1.05 mL, 19.7 mmol) in MeOH (140 mL) was stirred at 50° C. for 12 h. The mixture was adjusted to pH 7 with sat aq NaOH, then concentrated in vacuum. The residue was purified by reverse phase flash chromatography (water (0.1% TFA)-MeCN) to give the title compound (1.97 g, 98.9% yield) as a white solid.

LCMS: m/z 304.1 (M+H)⁺ (ES⁺).

¹H NMR (DMSO-d₆): δ 7.43 (d, 1H), 7.23 (dd, 1H), 6.92 (dd, 1H), 6.14 (s, 1H), 6.04 (dd, 1H), 3.64 (s, 2H), 3.59 (s, 3H), 3.01-2.98 (m, 1H), 1.17 (d, 6H). One exchangeable proton not observed.

### Step C: methyl 2-(4-fluoro-2-isopropyl-6-(2-(((trifluoromethyl)sulfonyl)oxy)pyridin-4-yl)phenyl)acetate

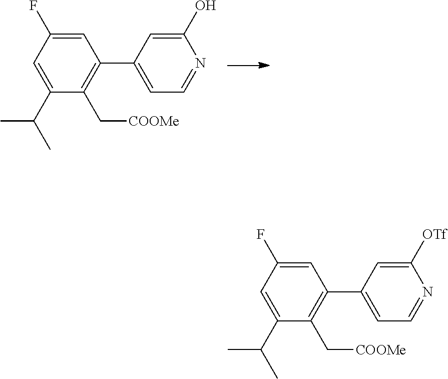 [SMILES: COC(=O)Cc1c(-c2ccnc(O)c2)cc(F)cc1C(C)C ; COC(=O)Cc1c(-c2ccnc(OS(=O)(=O)C(F)(F)F)c2)cc(F)cc1C(C)C]

To a solution of methyl 2-(4-fluoro-2-(2-hydroxypyridin-4-yl)-6-isopropylphenyl)-acetate (1.4 g, 4.62 mmol) in DCM (20 mL) was added TEA (1.87 g, 18.5 mmol) and Tf₂O (1.95 g, 6.92 mmol) at 0° C. The mixture was stirred at 25° C. for 12 h, then quenched with 1 M aq HCl (10 mL) and extracted with DCM (15 mL×2). The organic phases were concentrated in vacuum. The residue was purified by FC (PE:EtOAc, 30:1 to 20:1) to give the title compound (1.6 g, 79.6% yield) as a colourless oil.

LCMS: m/z 436.0 (M+H)⁺ (ES⁺).

¹H NMR (CDCl₃): δ 8.44 (d, 1H), 7.36 (dd, 1H), 7.19 (s, 1H), 7.14 (dd, 1H), 6.78 (dd, 1H), 3.67 (s, 3H), 3.52 (s, 2H), 3.07-3.04 (m, 1H), 1.23 (d, 6H).

### Intermediate C1: 2-(2-(2-((5-(5-((dimethylamino)methyl)-3-sulfamoyl-1H-pyrazol-1-yl)pentyl)oxy)pyridin-4-yl)-6-isopropylphenyl)acetic acid

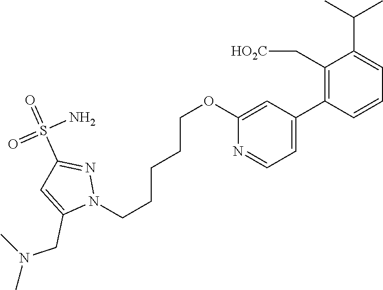 [SMILES: CC(C)c1cccc(-c2ccnc(OCCCCCn3nc(S(N)(=O)=O)cc3CN(C)C)c2)c1CC(=O)O]

Step A: 2-(2-(2-((5-(3-(N,N-bis(4-methoxybenzyl)sulfamoyl)-5-((dimethylamino)-methyl)-1H-pyrazol-1-yl)pentyl)oxy)pyridin-4-yl)-6-isopropylphenyl)acetic acid

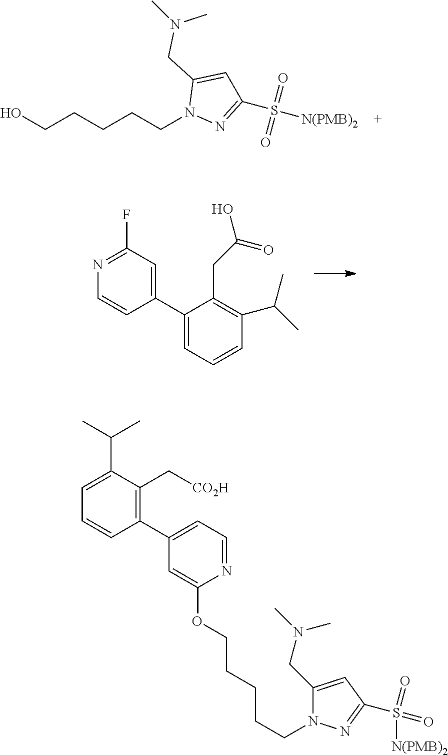

5-((Dimethylamino)methyl)-1-(5-hydroxypentyl)-N,N-bis(4-methoxybenzyl)-1H-pyrazole-3-sulfonamide (Intermediate A3) (416 mg, 0.784 mmol) was dissolved in THF (5 mL) and treated with NaH (60 wt % dispersion in mineral oil, 32 mg, 0.80 mmol). The reaction was stirred at RT for 1 h, then cooled to 0° C. 2-(2-(2-Fluoropyridin-4-yl)-6-isopropylphenyl)acetic acid (Intermediate B1) (214 mg, 0.784 mmol) was added and the reaction allowed to warm to RT and stirred for 3 h. The reaction was diluted with brine (30 mL), extracted with EtOAc (2×30 mL), dried (phase separator) and concentrated in vacuo. The crude product was purified by FC (0-10% (0.7 M ammonia/MeOH)/DCM) to afford the title compound (360 mg, 47%) as a grey solid.

LCMS m/z 784.3 (M+H)⁺ (ES⁺); 782.3 (M−H)⁻ (ES⁻).

¹H NMR (DMSO-d₆) δ 8.16 (d, J=5.2 Hz, 1H), 7.41-7.38 (m, 1H), 7.32 (t, J=7.7 Hz, 1H), 7.04-6.98 (m, 5H), 6.87 (d, J=5.2 Hz, 1H), 6.82-6.87 (m, 4H), 6.64 (s, 1H), 6.58 (s, 1H), 4.27 (t, J=6.6 Hz, 2H), 4.25-4.16 (m, 6H), 3.70 (s, 6H), 3.50 (s, 2H), 3.47 (s, 2H), 3.04 (sept, J=6.6 Hz, 1H), 2.15 (s, 6H), 1.85 (p, J=7.5 Hz, 2H), 1.76 (p, J=7.5 Hz, 2H), 1.48-1.36 (m, 2H), 1.19 (d, J=6.7 Hz, 6H). One exchangeable proton not observed.

### Step B: 2-(2-(2-((5-(5-((dimethylamino)methyl)-3-sulfamoyl-1H-pyrazol-1-yl)pentyl)-oxy)pyridin-4-yl)-6-isopropylphenyl)acetic acid

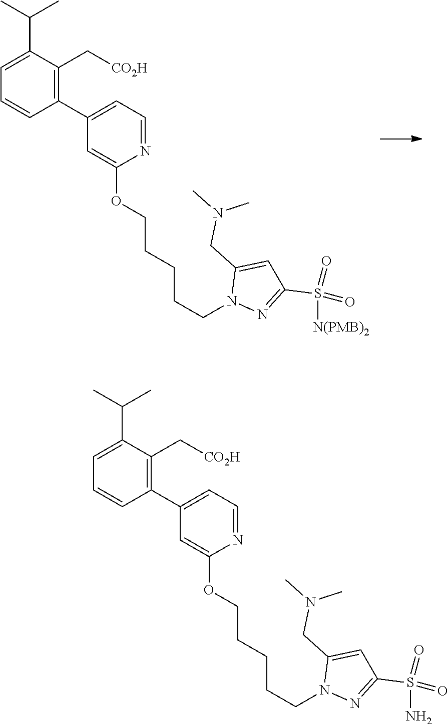

A solution of 2-(2-(2-((5-(3-(N,N-bis(4-methoxybenzyl)sulfamoyl)-5-((dimethyl-amino)methyl)-1H-pyrazol-1-yl)pentyl)oxy)pyridin-4-yl)-6-isopropylphenyl)acetic acid (360 mg, 0.367 mmol) in TFA (5 mL) was stirred at RT for 18 h. The mixture was diluted with MeOH (1 mL) and filtered. The resulting filtrate was concentrated in vacuo, re-dissolved in MeOH (5 mL) and filtered. The filtrate was concentrated in vacuo to afford the title compound (250 mg, 95%) as a yellow oil.

LCMS m/z 544.2 (M+H)⁺ (ES⁺); 542.1 (M−H)⁻ (ES⁻).

¹H NMR (DMSO-d₆) δ 10.08 (br s, 1H), 8.18 (d, J=5.2 Hz, 1H), 7.54 (br s, 2H), 7.44-7.39 (m, 1H), 7.34 (t, J=7.7 Hz, 1H), 7.04 (dd, J=7-5, 1.4 Hz, 1H), 6.88 (dd, J=5.2, 1-3 Hz, 1H), 6.85 (s, 1H), 6.68-6.65 (m, 1H), 4.53 (s, 2H), 4.32-4.23 (m, 4H), 3.17 (s, 2H), 3.04 (sept, J=6.7 Hz, 1H), 2.81 (s, 6H), 1.92-1.71 (m, 4H), 1.44 (p, J=7.6 Hz, 2H), 1.20 (d, J=6.8 Hz, 6H).

The following intermediates were synthesised following the general procedure for Intermediate C1, from the intermediate compounds indicated in the ‘From’ column:

| Int | Structure | From | ¹H NMR | LCMS |
|---|---|---|---|---|
| C2 | 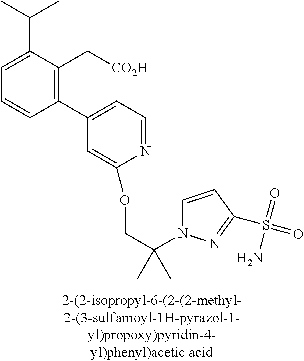 [SMILES: CC(C)c1cccc(-c2ccnc(OCC(C)(C)n3ccc(S(N)(=O)=O)n3)c2)c1CC(=O)O] | A1 + B1 | ¹H NMR (DMSO-d₆) δ 8.17 (d, J = 5.2 Hz, 1H), 8.03 (d, J = 2.5 Hz, 1H), 7.42-7.37 (m, 3H), 7.33 (t, J = 7.7 Hz, 1H), 7.03 (dd, J = 7.5, 1.4 Hz, 1H), 6.91 (dd, J = 5.2, 1.4 Hz, 1H), 6.66-6.65 (m, 1H), 6.59 (d, J = 2.4 Hz, 1H), 4.55 (s, 2H), 3.04 (app p), J = 6.7 Hz, 1H), 1.66 (s, 6H), 1.19 (d, J = 6.7 Hz, 6H). One exchangeable proton not observed and two masked by water peak. | m/z 473.2 (M + H)⁺ (ES⁺) |
| C3 | 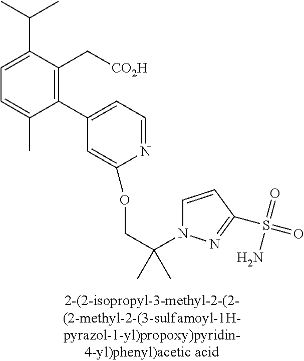 [SMILES: Cc1ccc(C(C)C)c(CC(=O)O)c1-c1ccnc(OCC(C)(C)n2ccc(S(N)(=O)=O)n2)c1] | A1 + B2 | ¹H NMR (DMSO-d₆) δ 8.20 (d, J = 5.2 Hz, 1H), 8.02 (d, J =2.4 Hz, 1H), 7.40 (br s, 2H), 7.26 (d, J = 8.0 Hz, 1H), 7.19 (d, J = 7.9 Hz, 1H), 6.75 (dd, J = 5.2, 1.4 Hz, 1H), 6.58 (d, J = 2.4 Hz, 1H), 6.54- 6.51 (m, 1H), 4.58 (s, 2H), 3.30 (d, J = 5.8 Hz, 2H), 2.99-2.94 (m, 1H), 1.91 (s, 3H), 1.66, 1.65 (2 × s, 2 × 3H), 1.17 (d, J = 6.8 Hz, 6H). One exchangeable proton not observed. | — |
| C4 | 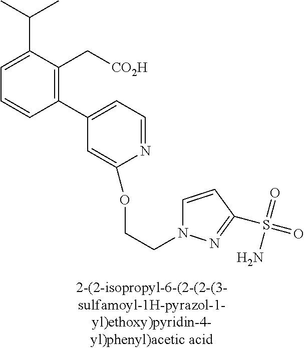 [SMILES: CC(C)c1cccc(-c2ccnc(OCCn3ccc(S(N)(=O)=O)n3)c2)c1CC(=O)O] | A2 + B1 | ¹H NMR (DMSO-d₆) δ 8.21 (d, J = 5.2 Hz, 1H), 7.92 (d, J = 2.3 Hz, 1H), 7.44-7.39 (m, 3H), 7.34 (t, J = 7.7 Hz, 1H), 7.03 (dd, J = 7.5, 1.4 Hz, 1H), 6.93 (dd, J = 5.2, 1.4 Hz, 1H), 6.68 (s, 1H), 6.59 (d, J = 2.3 Hz, 1H), 4.71- 4.65 (m, 2H), 4.63-4.58 (m, 2H), 3.51 (s, 2H), 3.06-3.00 (m, 1H), 1.20 (d, J = 6.8 Hz, 6H). One exchangeable proton not observed. | m/z 445.1 (M + H)⁺ (ES⁺) |
| C5 | 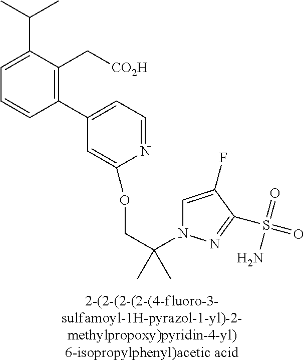 [SMILES: CC(C)c1cccc(-c2ccnc(OCC(C)(C)n3cc(F)c(S(N)(=O)=O)n3)c2)c1CC(=O)O] | A4 + B1 | ¹H NMR (DMSO-d₆) δ 8.24 (d, J = 4.5 Hz, 1H), 8.16 (d, J = 5.2 Hz, 1H), 7.69 (s, 2H), 7.40 (dd, J = 7.9, 1.4 Hz, 1H), 7.32 (t, J = 7.7 Hz, 1H), 7.03 (dd, J = 7.5, 1.4 Hz, 1H), 6.91 (dd, J = 5.2, 1.5 Hz, 1H), 6.68 (d, J = 1.4 Hz, 1H), 4.52 (s, 2H), 3.50 (s, 2H), 3.03 (sept, J = 6.7 Hz, 1H), 1.62 (s, 6H), 1.19 (d, J = 6.8 Hz, 6H). One exchangeable proton not observed. | m/z 491.2 (M + H)⁺ (ES ⁺) 489.2 (M − H)⁺ (ES⁻) |
| C27 | 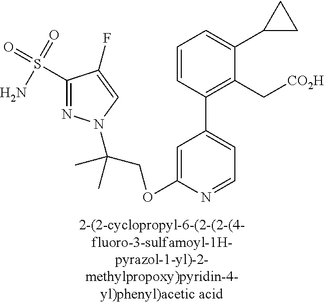 [SMILES: CC(C)(COc1cc(-c2cccc(C3CC3)c2CC(=O)O)ccn1)n1cc(F)c(S(N)(=O)=O)n1] | A4 + B6 | ¹H NMR (DMSO-d₆) δ 8.26-8.23 (m, 1H), 8.17 (dd, J = 5.2, 0.7 Hz, 1H), 7.69 (s, 2H) 7.26 (t, J = 7.7 Hz, 1H), 7.11 (dd, J = 7.8, 1.4 Hz, 1H), 7.06 (dd, J = 7.6, 1.4 Hz, 1H), 6.93 (dd, J = 5.2, 1.5 Hz, 1H), 6.69 (m, 1H) 4.53 (d, J = 3.2 Hz, 2H), 3.64 (s, 2H), 1.88 (ddt, J = 10.8, 8.4, 3.8 Hz, 1H), 1.63 (s, 6H), 0.97-0.87 (m, 2H), 0.69-0.59 (m, 2H). One exchangeable proton not observed. | m/z 489.1 (M + H)⁺ (ES⁺) |
| C28 | 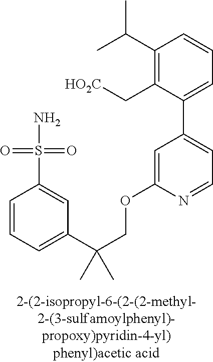 [SMILES: CC(C)c1cccc(-c2ccnc(OCC(C)(C)c3cccc(S(N)(=O)=O)c3)c2)c1CC(=O)O] | A17 + B1 | ¹H NMR (DMSO-d₆) δ 12.42 (s, 1H), 8.18-8.12 (m, 1H), 7.93 (t, J = 1.9 Hz, 1H), 7.73-7.65 (m, 2H), 7.52 (t, J = 7.9 Hz, 1H), 7.42-7.36 (m, 1H), 7.33-7.24 (m, 3H), 7.02 (dd, J = 7.5, 1.4 Hz, 1H), 6.87 (dd, J = 5.2, 1.5 Hz, 1H), 6.64-6.58 (m, 1H), 4.37 (s, 2H), 3.49 (s, 2H), 3.02 (sept, J = 6.8 Hz, 1H), 1.43 (s, 6H), 1.18 (d, J = 6.8 Hz, 6H). | m/z 483.5 (M + H)⁺ (ES⁺) 481.4 (M − H)⁻ (ES⁻) |
| C29 | 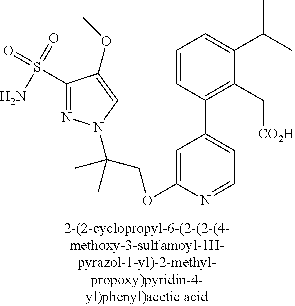 [SMILES: COc1cn(C(C)(C)COc2cc(-c3cccc(C(C)C)c3CC(=O)O)ccn2)nc1S(N)(=O)=O] | A11 + B1 | ¹H NMR (CD₃OD) δ 8.14 (d, J = 5.3 Hz, 1H), 7.72 (s, 1H), 7.44-7.40 (m, 1H), 7.35 (t, J = 7.7 Hz, 1H), 7.05 (dd, J = 7.5, 1.4 Hz, 1H), 6.98 (dd, J = 5.3, 1.5 Hz, 1H), 6.81 (d, J = 1.4 Hz, 1H), 4.59 (s, 2H), 3.81 (s, 3H), 3.65- 3.54 (m, 2H), 3.14 (sept, J = 6.8 Hz, 1H), 1.72 (s, 6H), 1.27 (d, J = 6.8 Hz, 6H). Three exchangeable protons not observed. | m/z 503.5 (M + H)⁺ (ES⁺) |
| C30 | 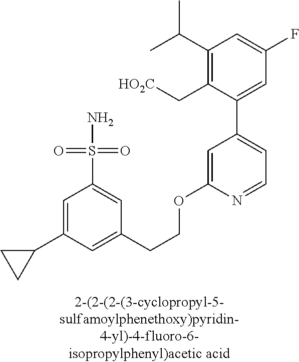 [SMILES: CC(C)c1cc(F)cc(-c2ccnc(OCCc3cc(C4CC4)cc(S(N)(=O)=O)c3)c2)c1CC(=O)O] | A18 + B4 | ¹H NMR (DMSO-d₆) δ 8.22 (d, J = 5.2 Hz, 1H), 7.54 (t, J = 1.7 Hz, 1H), 7.38 (t, J = 1.7 Hz, 1H), 7.29-7.20 (m, 4H), 6.96- 6.84 (m, 2H), 6.69 (s, 1H), 4.52 (t, J = 6.9 Hz, 2H), 3.48 (s, 2H), 3.10 (t, J = 6.8 Hz, 2H), 3.04 (sept, J = 6.9 Hz, 1H), 2.05-1.97 (m, 1H), 1.19 (d, J = 6.8 Hz, 6H), 1.05- 0.97 (m, 2H), 0.73-0.68 (m, 2H). One exchangeable proton not observed. | m/z 513.4 (M + H)⁺ (ES⁺) 511.3 (M − H)⁻ (ES⁻) |
| C31 | 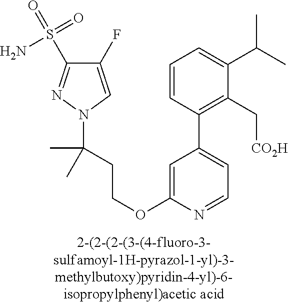 [SMILES: CC(C)c1cccc(-c2ccnc(OCCC(C)(C)n3cc(F)c(S(N)(=O)=O)n3)c2)c1CC(=O)O] | A19 + B1 | ¹H NMR (DMSO-d₆) δ 8.26 (d, J = 4.5 Hz, 1H), 8.16 (d, J = 5.2 Hz, 1H), 7.69 (s, 2H), 7.40 (dd, J = 7.9, 1.4 Hz, 1H), 7.33 (t, J = 7.7 Hz, 1H), 7.03 (dd, J = 7.5, 1.4 Hz, 1H), 6.87 (dd, J = 5.2, 1.4 Hz, 1H), 6.62 (s, 1H), 4.15 (t, J = 7.0 Hz, 2H), 3.50 (s, 2H), 3.03 (p, J = 6.8 Hz, 1H), 1.60 (s, 6H), 1.20-1.15 (m, 8H). One exchangeable proton not observed. | m/z 505.4 (M + H)⁺ (ES⁺) 503.4 (M − H)⁻ (ES⁻) |
| C32 | 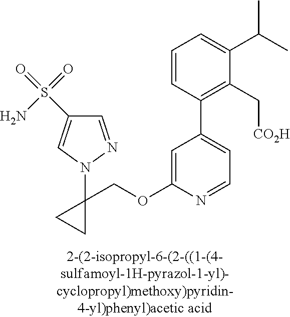 [SMILES: CC(C)c1cccc(-c2ccnc(OCC3(n4cc(S(N)(=O)=O)cn4)CC3)c2)c1CC(=O)O] | A20 + B1 | ¹H NMR (DMSO-d₆) δ 8.26 (s, 1H), 8.15 (d, J = 5.2 Hz, 1H), 7.73 (s, 1H), 7.43-7.38 (m, 1H), 7.33 (t, J = 7.7 Hz, 1H), 7.23 (s, 2H), 7.05-6.99 (m, 1H), 6.89 (dd, J = 5.1, 1.4 Hz, 1H), 6.67 (s, 1H), 4.60 (s, 2H), 3.50 (s, 2H), 3.10-2.96 (m, 1H), 1.40-1.26 (m, 4H), 1.19 (d, J = 6.7 Hz, 6H). One exchangeable proton not observed. | m/z 471.4 (M + H)⁺ (ES⁺) |
| C33 | 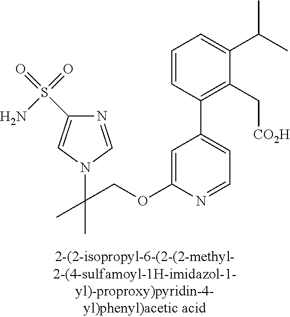 [SMILES: CC(C)c1cccc(-c2ccnc(OCC(C)(C)n3cnc(S(N)(=O)=O)c3)c2)c1CC(=O)O] | A21 + B1 | ¹H NMR (DMSO-d₆) δ 12.43 (s, 1H), 8.17 (d, J = 5.2 Hz, 1H), 7.99 (d, J = 1.5 Hz, 1H), 7.85 (d, J = 1.5 Hz, 1H), 7.40 (dd, J = 7.9, 1.4 Hz, 1H), 7.32 (t, J = 7.7 Hz, 1H), 7.12 (s, 2H), 7.03 (dd, J = 7.5, 1.4 Hz, 1H), 6.91 (dd, J = 5.1, 1.4 Hz, 1H), 6.70 (s, 1H), 4.49 (s, 2H), 3.50 (s, 2H), 3.03 (p, J = 6.7 Hz, 1H), 1.65 (s, 6H), 1.19 (d, J = 6.7 Hz, 6H). | m/z 473.4 (M + H)⁺ (ES⁺) 471.3 (M − H)⁻ (ES⁻) |
| C36 | 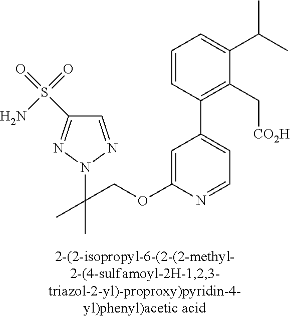 [SMILES: CC(C)c1cccc(-c2ccnc(OCC(C)(C)n3ncc(S(N)(=O)=O)n3)c2)c1CC(=O)O] | A22 + B1 | ¹H NMR (DMSO-d₆) δ 12.43 (s, 1H), 8.16 (d, J = 5.2 Hz, 1H), 8.10 (s, 1H), 7.78 (s, 2H), 7.39 (dd, J = 8.0, 1.4 Hz, 1H), 7.31 (t, J = 7.7 Hz, 1H), 7.02 (dd, J = 7.5, 1.4 Hz, 1H), 6.90 (dd, J = 5.2, 1.4 Hz, 1H), 6.60 (d, J = 1.3 Hz, 1H), 4.71 (s, 2H), 3.49 (s, 2H), 3.02 (sept, J = 6.8 Hz, 1H), 1.74 (s, 6H), 1.18 (d, J = 6.7 Hz, 6H). | m/z 474.5 (M + H)⁺ (ES⁺) 472.0 (M − H)⁻ (ES⁻) |
| C37 | 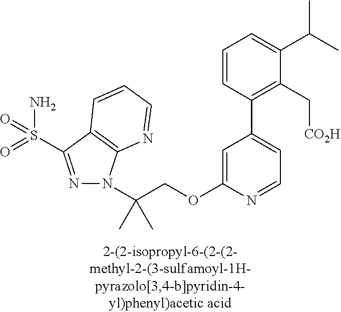 [SMILES: CC(C)c1cccc(-c2ccnc(OCC(C)(C)n3nc(S(N)(=O)=O)c4cccnc43)c2)c1CC(=O)O] | A23 + B1 | ¹H NMR (DMSO-d₆) δ 8.66 (dd, J = 4.5, 1.6 Hz, 1H), 8.40 (dd, J = 8.2, 1.7 Hz, 1H), 8.09 (d, J = 5.2 Hz, 1H), 7.82 (s, 2H), 7.42 (dd, J = 8.1,4.5 Hz, 1H), 7.37 (d, J = 7.8 Hz, 1H), 7.29 (t, J = 7.7 Hz, 1H), 7.00-6.93 (m, 1H), 6.84 (dd, J = 5.2, 1.4 Hz, 1H), 6.52 (s, 1H), 5.01 (s, 2H), 3.45 (s, 2H), 3.06- 2.95 (m, 1H), 1.91 (s, 6H), 1.17 (d, J = 6.8 Hz, 6H). One exchangeable proton not observed. | m/z 524.4 (M + H)⁺ (ES⁺) |
| C38 | 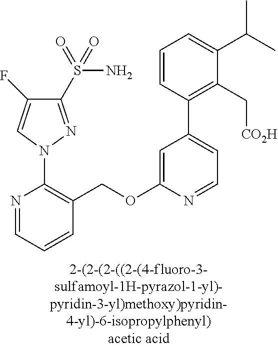 [SMILES: CC(C)c1cccc(-c2ccnc(OCc3cccnc3-n3cc(F)c(S(N)(=O)=O)n3)c2)c1CC(=O)O] | A24 + B1 | ¹H NMR (DMSO-d₆) δ 8.83 (d, J = 4.5 Hz, 1H), 8.52-8.50 (m, 1H), 8.10 (d, J = 5.3 Hz, 1H), 8.09- 8.05 (m, 1H), 7.98 (s, 2H), 7.55 (dd, J = 7.8, 4.7 Hz, 1H), 7.42 (d, J = 7.9 Hz, 1H), 7.35 (t, J = 7.7 Hz, 1H), 7.09-7.05 (m, 1H), 6.91 (dd, J = 5.2, 1.4 Hz, 1H), 6.84 (s, 1H), 5.77 (s, 2H), 3.52 (s, 2H), 3.05 (m, 1H), 3.52 (s, 6H). One exchangeable proton not observed. | — |

### Intermediate C6: 2-(5-(2-(2-(4-fluoro-3-sulfamoyl-1H-pyrazol-1-yl)-2-methylpropoxy)pyridin-4-yl)-2,3-dihydro-1H-inden-4-yl)acetic acid

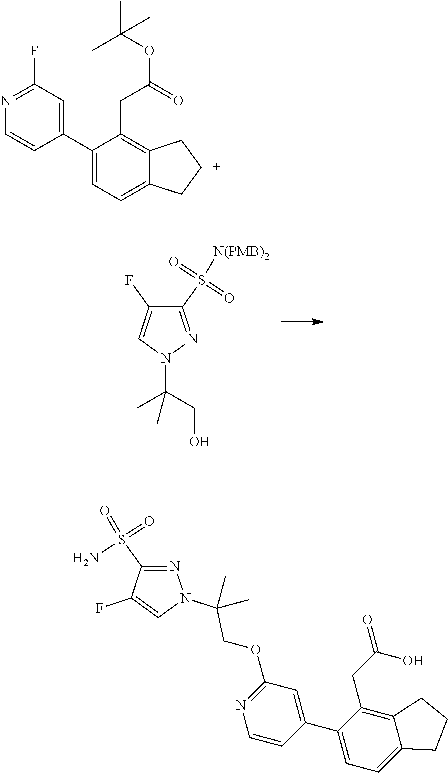

KO^{t}Bu (172 mg, 1.529 mmol) was added to a solution of tert-butyl 2-(5-(2-fluoro-pyridin-4-yl)-2,3-dihydro-1H-inden-4-yl)acetate (Intermediate B13) (250 mg, 0.764 mmol) and 4-fluoro-1-(1-hydroxy-2-methylpropan-2-yl)-N,N-bis(4-methoxybenzyl)-1H-pyrazole-3-sulfonamide (Intermediate A4) (365 mg, 0.764 mmol) in THF (12 mL) at 0° C. The mixture was stirred at RT for 48 h before additional KO^{t}Bu (56 mg, 0.766 mmol) was added and stirred for 4 h. The mixture was diluted with water (10 mL) and DCM (10 mL). The aqueous phase was extracted with DCM (3×10 mL) and the combined organic phases were dried (MgSO₄) and concentrated in vacuo. TFA (5 mL) was added to the residue and stirred at RT for 16 h. The mixture was concentrated in vacuo. MeOH (2 mL) was added and the mixture evaporated in vacuo. The crude was purified by FC (0-40% MeOH/DCM) to afford the title compound (187 mg, 50%) as a white solid.

LCMS m/z 489.1 (M+H)⁺ (ES⁺).

¹H NMR (DMSO-d₆) δ 8.26 (d, J=4.6 Hz, 1H), 8.16 (d, J=5.2 Hz, 1H), 7.70 (s, 2H), 7.22 (d, J=7.7 Hz, 1H), 7.03 (d, J=7.6 Hz, 1H), 6.91 (dd, J=5.3, 1.4 Hz, 1H), 6.68 (s, 1H), 4.53 (s, 2H), 3.46 (s, 2H), 2.94 (t, J=7.5 Hz, 2H), 2.83 (t, J=7.5 Hz, 2H), 2.05 (p, J=7.4 Hz, 3H), 1.63 (s, 6H).

The following intermediates ‘Int.’ were synthesised following the general procedure for Intermediate C6, from the intermediate compounds indicated in the ‘From’ column:

| Int. | Structure | From | ¹H NMR | LCMS |
|---|---|---|---|---|
| C7 | 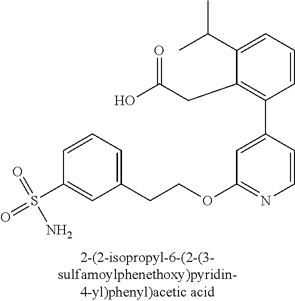 [SMILES: CC(C)c1cccc(-c2ccnc(OCCc3cccc(S(N)(=O)=O)c3)c2)c1CC(=O)O] | A6 + B1 | ¹H NMR (DMSO-d₆) δ 12.43 (br s, 1H), 8.19 (d, J = 5.2 Hz, 1H), 7.79 (d, J = 1.8 Hz, 1H), 7.69 (dt, J = 7.5, 1.7 Hz, 1H), 7.56-7-53 (m, 1H), 7.51-7.48 (m, 1H), 7.40 (dd, J = 7.9, 1.4 Hz, 1H), 7.33-7.30 (m, 3H), 7.03 (dd, J = 7.5, 1.4 Hz, 1H), 6.89 (dd, J = 5.2, 1.4 Hz, 1H), 6.71-6.59 (m, 1H), 4-53 (t, J = 6.8 Hz, 2H), 3.51 (s, 2H), 3.20- 3.08 (m, 2H), 3.03 (p, J = 6.8 Hz, 1H), 1.19 (d, J = 6.8 Hz, 6H). | m/z 455.0 (M + H)⁺ (ES⁺) 453.4 (M − H)⁻ (ES⁻) |
| C8 | 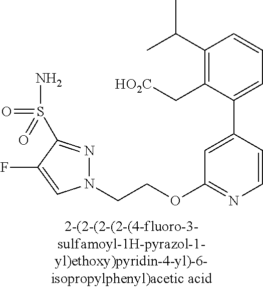 [SMILES: CC(C)c1cccc(-c2ccnc(OCCn3cc(F)c(S(N)(=O)=O)n3)c2)c1CC(=O)O] | A8 + B1 | ¹H NMR (DMSO-d₆) δ 8.20 (d, J = 5.3 Hz, 1H), 8.12 (d, J = 4.6 Hz, 1H), 7.72 (s, 2H), 7.41 (d, J = 7.9 Hz, 1H), 7.34 (t, J = 7.6 Hz, 1H), 7.04 (dd, J = 7.4, 1.5 Hz, 1H), 6.93 (dd, J = 5.2, 1.5 Hz, 1H), 6.71 (d, J = 1.4 Hz, 1H), 4.66 (t, J = 5.1 Hz, 2H), 4.53 (d, J = 5.3 Hz, 2H), 3.51 (s, 2H), 3.05 (d, J = 6.7 Hz, 1H), 1.20 (d, J = 6.7 Hz, 6H). One exchangeable proton not observed. | m/z 463.2 (M + H)⁺ (ES⁺) |
| C9 | 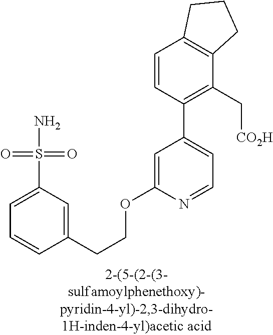 [SMILES: NS(=O)(=O)c1cccc(CCOc2cc(-c3ccc4c(c3CC(=O)O)CCC4)ccn2)c1] | A6 + B3 | ¹H NMR (DMSO-d₆) δ 8.19 (d, J = 5.2 Hz, 1H), 7.81- 7.77 (m, 1H), 7.69 (dt, J = 7.5, 1.6 Hz, 1H), 7.56-7-53 (m, 1H), 7.51 (t, J = 7.6 Hz, lH), 7.32 (br s, 2H), 7.21 (d, J = 7.7 Hz, 1H), 7.02 (d, J = 7.7 Hz, 1H), 6.89 (dd, J = 5.2, 1.5 Hz, 1H), 6.67-6.65 (m, 1H), 4.53 (t, J = 6.8 Hz, 2H), 3.46 (s, 2H), 3.15 (t, J = 6.9 Hz, 2H), 2.93 (t, J = 7.5 Hz, 2H), 2.82 (t, J = 7.5 Hz, 2H), 2.04 (p, J = 7.5 Hz, 2H). One exchangeable proton not observed. | m/z 453.4 (M + H)⁺ (ES⁺) |
| C10 | 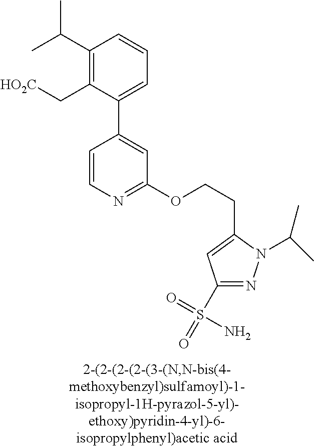 [SMILES: CC(C)c1cccc(-c2ccnc(OCCc3cc(S(N)(=O)=O)nn3C(C)C)c2)c1CC(=O)O] | A5 + B1 | ¹H NMR (DMSO-d₆) δ 12.44 (s, 1H), 8.22 (d, J = 5.1 Hz, 1H), 7.41 (d, J = 7.7 Hz, 1H), 7.37-7.30 (m, 3H), 7.04 (d, J = 7.6 Hz, 1H), 6.92 (d, J = 5.3 Hz, 1H), 6.69 (s, 1H), 6.50 (s, 1H), 4.78-4.68 (m, 1H), 4.54 (t, J = 6.4 Hz, 2H), 3.52 (s, 2H), 3.21 (t, J = 6.5 Hz, 2H), 3.07-2.98 (m, 1H), 1.40 (d, J = 6.5 Hz, 6H), 1.19 (d, J = 6.8 Hz, 6H). | m/z 487 (M + H)⁺ (ES⁺) |
| C12 | 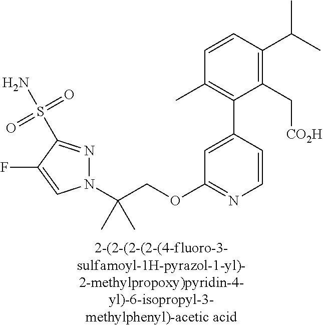 [SMILES: Cc1ccc(C(C)C)c(CC(=O)O)c1-c1ccnc(OCC(C)(C)n2cc(F)c(S(N)(=O)=O)n2)c1] | A4 + B2 | ¹H NMR (DMSO-d₆) δ 8.26-8.21 (m, 1H), 8.19 (d, J = 5.2 Hz, 1H), 7.70 (s, 2H), 7.27 (d, J = 8.0 Hz, 1H), 7.19 (d, J = 8.1 Hz, 1H), 6.75 (dd, J = 5.2, 1.4 Hz, 1H), 6.55 (s, 1H), 4.60-4.49 (m, 2H), 3.30 (s, 2H), 2.97 (p, J = 6.8 Hz, 1H), 1.91 (d, J = 2.1 Hz, 3H), 1.62 (d, J = 6.2 Hz, 6H), 1.17 (dd, J = 6.7, 1.7 Hz, 6H). One exchangeable proton not observed. | m/z 505.4 (M + H)⁺ (ES⁺) |
| C13 | 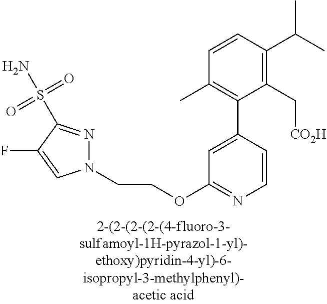 [SMILES: Cc1ccc(C(C)C)c(CC(=O)O)c1-c1ccnc(OCCn2cc(F)c(S(N)(=O)=O)n2)c1] | A8 + B2 | ¹H NMR (DMSO-d₆) δ 8.22 (d, J = 5.0 Hz, 1H), 8.11 (d, J = 4.7 Hz, 1H), 7.72 (s, 2H), 7.27 (d, J = 8.0 Hz, 1H), 7.19 (d, J = 8.1 Hz, 1H), 6.77 (dd, J = 5.2,1.4 Hz, 1H), 6.57 (s, 1H), 5.76 (s, 1H), 4.74- 4.61 (m, 2H), 4.56-4.51 (m, 2H), 3.02-2.92 (m, 2H), 1.91 (s, 3H), 1.17 (dd, J = 6.8, 2.5 Hz, 6H). One exchangeable proton not observed. | m/z 477.4 (M + H)⁺ (ES⁺) |
| C14 | 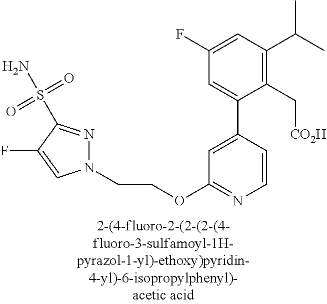 [SMILES: CC(C)c1cc(F)cc(-c2ccnc(OCCn3cc(F)c(S(N)(=O)=O)n3)c2)c1CC(=O)O] | A8 + B4 | ¹H NMR (DMSO-d₆) δ 8.22 (d, J = 5-2 Hz, 1H), 8.12 (d, J = 4.7 Hz, 1H), 7.72 (s, 2H), 6.96-6.89 (m, 2H), 6.73 (s, 1H), 4.66 (t, J = 5.2 Hz, 2H), 4.52 (t, J = 5.2 Hz, 2H), 3.48 (s, 2H), 3.08-2.99 (m, 1H), 1.19 (d, J = 6.7 Hz, 6H). 2 protons obscured by solvent signal. | m/z 481.4 (M + H)⁺ (ES⁺) |
| C15 | 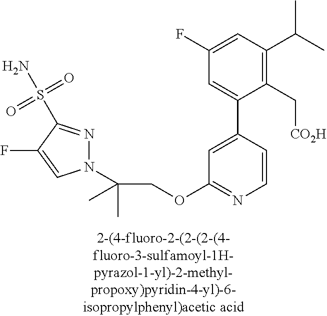 [SMILES: CC(C)c1cc(F)cc(-c2ccnc(OCC(C)(C)n3cc(F)c(S(N)(=O)=O)n3)c2)c1CC(=O)O] | A4 + B4 | ¹H NMR (DMSO-d₆) δ 8.25 (d, J = 4.6 Hz, 1H), 8.19 (dd, J = 5.2, 0.7 Hz, 1H), 7.70 (s, 2H), 7.24 (dd, J = 10.5, 2.8 Hz, 1H), 6.94- 6.90 (m, 2H), 6.71 (t, J = 1.1 Hz, 1H), 4.53 (s, 2H), 3.47 (s, 2H), 3.08-2.99 (m, 1H), 1.63 (s, 6H), 1.19 (d, J = 6.8 Hz, 6H). One exchangeable proton not observed. | m/z 509.4 (M + H)⁺ (ES⁺) |
| C16 | 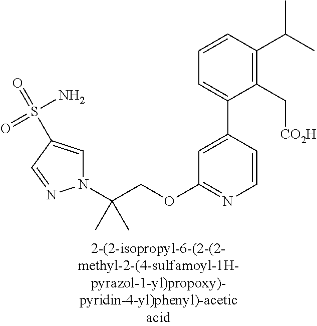 [SMILES: CC(C)c1cccc(-c2ccnc(OCC(C)(C)n3cc(S(N)(=O)=O)cn3)c2)c1CC(=O)O] | A9 + B1 | | m/z 473.4 (M + H)⁺ (ES⁺) |
| C18 | 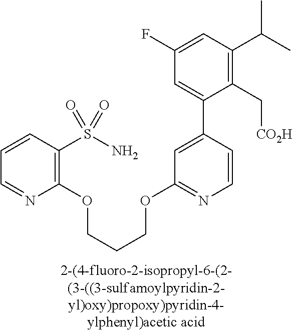 [SMILES: CC(C)c1cc(F)cc(-c2ccnc(OCCCOc3ncccc3S(N)(=O)=O)c2)c1CC(=O)O] | A10 + B4 | ¹H NMR (DMSO-d₆) δ 12.47 (s, 1H), 8.35 (dd, J = 5.0, 1.9 Hz, 1H), 8.19 (d, J = 5.2 Hz, 1H), 8.11 (dd, J = 7.5, 1.9 Hz, 1H), 7.30 (s, 2H), 7.24 (dd, J = 10.4, 2.7 Hz, 1H), 7.15 (dd, J = 7.5, 5.0 Hz, 1H), 6.95-6.85 (m, 2H), 6.71 (d, J = 1.3 Hz, 1H), 4.58 (t, J = 6.2 Hz, 2H), 4.48 (t, J = 6.3 Hz, 2H), 3.48 (s, 2H), 3.09- 2.97 (m, 1H), 2.28 (t, J = 6.3 Hz, 2H), 1.19 (d, J = 6.8 Hz, 6H). | m/z 504.4 (M + H)⁺ (ES⁺) |
| C19 | 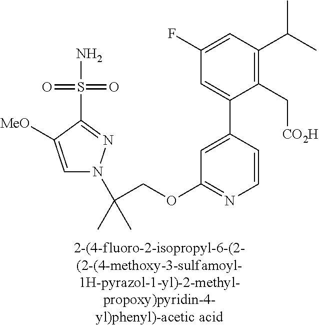 [SMILES: COc1cn(C(C)(C)COc2cc(-c3cc(F)cc(C(C)C)c3CC(=O)O)ccn2)nc1S(N)(=O)=O] | A11 + B4 | ¹H NMR (DMSO-d₆) δ 8.19 (d, J = 5.2 Hz, 1H), 7.85 (s, 1H), 7.29 (s, 2H), 7.23 (dd, J = 10.5, 2.8 Hz, 1H), 6.94- 6.89 (m, 2H), 6.71 (d, J = 1.2 Hz, 1H), 4.52 (s, 2H), 3.72 (s, 3H), 3.47 (s, 2H), 3.10-2.92 (m, 1H), 1.62 (s, 6H), 1.18 (d, J = 6.7 Hz, 6H). One exchangeable proton not observed. | m/z 520.8 (M + H)⁺ (ES⁺) |
| C20 | 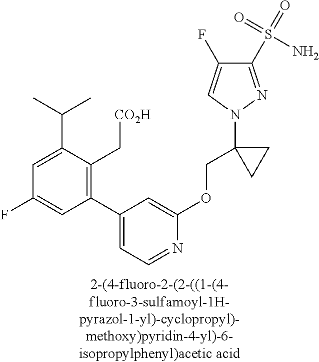 [SMILES: CC(C)c1cc(F)cc(-c2ccnc(OCC3(n4cc(F)c(S(N)(=O)=O)n4)CC3)c2)c1CC(=O)O] | A12 + B4 | ¹H NMR (DMSO-d₆) δ 12.47 (s, 1H), 8.23-8.09 (m, 2H), 7.73 (s, 2H), 7.23 (dd, J = 10.5, 2.8 Hz, 1H), 6.95-6.83 (m, 2H), 6.72 (dd, J = 1.5, 0.7 Hz, 1H), 4.57 (s, 2H), 3.47 (s, 2H), 3.08-2.96 (m, 1H), 1.40- 1.23 (m, 4H), 1.18 (d, J = 6.7 Hz, 6H). | m/z 507.4 (M + H)⁺ (ES⁺) |
| C21 | 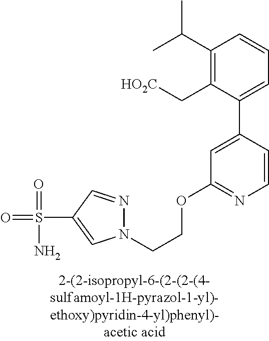 [SMILES: CC(C)c1cccc(-c2ccnc(OCCn3cc(S(N)(=O)=O)cn3)c2)c1CC(=O)O] | A14 + B1 | ¹H NMR (DMSO-d₆) δ 8.25 (s, 1H), 8.20 (d, J = 5.2 Hz, 1H), 7.74 (s, 1H), 7.40 (dd, J = 7.9, 1.4 Hz, 1H), 7.33 (t, J = 7.7 Hz, 1H), 7.24 (s, 2H), 7.02 (dd, J = 7.5, 1.4 Hz, 1H), 6.91 (dd, J = 5.2, 1.4 Hz, 1H), 6.68-6.64 (m, 1H), 4.67 (t, J = 5.2 Hz, 2H), 4.57 (t, J = 5.2 Hz, 2H), 3.50 (s, 2H), 3.10-2.95 (m, 1H), 1.19 (d, J = 6.8 Hz, 6H). One exchangeable proton not observed. | m/z 445.8 (M + H)⁺ (ES⁺) |
| C22 | 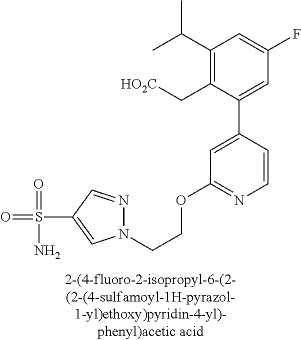 [SMILES: CC(C)c1cc(F)cc(-c2ccnc(OCCn3cc(S(N)(=O)=O)cn3)c2)c1CC(=O)O] | A14 + B4 | ¹H NMR (DMSO-d₆) δ 12.47 (s, 1H), 8.25 (s, 1H), 8.22-8.20 (m, 1H), 7.74 (s, 1H), 7.30-7.15 (m, 3H), 6.99-6.81 (m, 2H), 6.71-6.63 (m, 1H), 4.70- 4.63 (m, 2H), 4.57 (t, J = 5.2 Hz, 2H), 3.47 (s, 2H), 3.09-2.95 (m, 1H), 1.18 (d, J = 6.8 Hz, 6H). | m/z 463.5 (M + H)⁺ (ES⁺) |
| C23 | 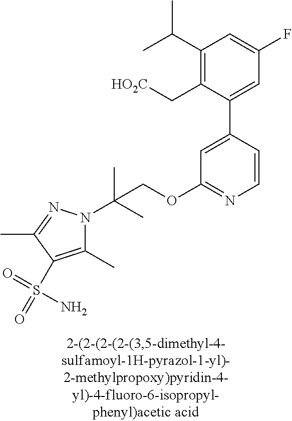 [SMILES: Cc1nn(C(C)(C)COc2cc(-c3cc(F)cc(C(C)C)c3CC(=O)O)ccn2)c(C)c1S(N)(=O)=O] | A13 + B4 | ¹H NMR (DMSO-d₆) δ 8.19 (d, J = 5.1 Hz, 1H), 7.27- 7.21 (m, 1H), 7.05 (s, 2H), 6.94-6.89 (m, 2H), 6.69 (s, 1H), 4.60 (s, 2H), 3.47 (s, 2H), 3.03 (d, J = 23.3 Hz, 1H), 2.64-2.63 (m, 3H), 2.23 (s, 3H), 1.69 (s, 6H), 1.19 (d, J = 6.7 Hz, 6H). One exchangeable proton not observed. | m/z 519.4 (M + H)⁺ (ES⁺) |
| C24 | 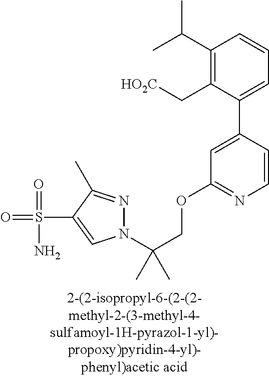 [SMILES: Cc1nn(C(C)(C)COc2cc(-c3cccc(C(C)C)c3CC(=O)O)ccn2)cc1S(N)(=O)=O] | A15 + B1 | ¹H NMR (DMSO-d₆) δ 8.17 (dd, J = 5.2, 0.7 Hz, 1H), 8.09 (s, 1H), 7.39 (dd, J = 7.9, 1.4 Hz, 1H), 7.32 (t, J = 7.7 Hz, 1H), 7.15 (s, 2H), 7.03 (dd, J = 7.5, 1.4 Hz, 1H), 6.90 (dd, J = 5.2, 1.5 Hz, 1H), 6.68-6.62 (m, 1H), 4.53 (s, 2H), 3.50 (s, 2H), 3.08-2.97 (m, 1H), 2.29 (s, 3H), 1.60 (s, 6H), 1.19 (d, J = 6.8 Hz, 6H). One exchangeable proton not observed. | m/z 487.6 (M + H)⁺ (ES⁺) |
| C25 | 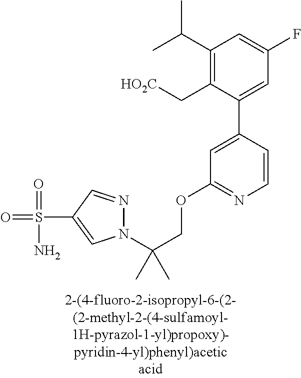 [SMILES: CC(C)c1cc(F)cc(-c2ccnc(OCC(C)(C)n3cc(S(N)(=O)=O)cn3)c2)c1CC(=O)O] | A9 + B4 | ¹H NMR (DMSO-d₆) δ 12.46 (s, 1H), 8.26 (d, J = 0.7 Hz, 1H), 8.18 (dd, J = 5.2, 0.7 Hz, 1H), 7.76 (d, J = 0.7 Hz, 1H), 7.25-7.18 (m, 3H), 6.93-6.88 (m, 2H), 6.66 (dd, J = 1.4, 0.7 Hz, 1H), 4.57 (s, 2H), 3.46 (s, 2H), 3.07-2.97 (m, 1H), 1.64 (s, 6H), 1.18 (d, J = 6.8 Hz, 6H). | m/z 491.5 (M + H)⁺ (ES⁺) |
| C34 | 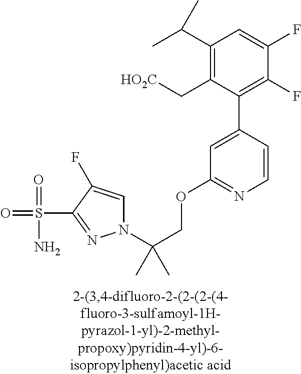 [SMILES: CC(C)c1cc(F)c(F)c(-c2ccnc(OCC(C)(C)n3cc(F)c(S(N)(=O)=O)n3)c2)c1CC(=O)O] | A4 + B7 | ¹H NMR (DMSO-d₆) δ 8.25 (d, J = 4.9 Hz, 2H), 7.70 (s, 2H), 7.52-7.41 (m, 1H), 6.92-6.87 (m, 1H), 6.74 (s, 1H), 4.58- 4.49 (m, 2H), 3.39 (s, 2H), 3.06-2.95 (m, 1H), 1.62 (s, 6H), 1.17 (d, J = 6.7 Hz, 6H). One exchangeable proton not observed. | m/z 527.3 (M + H)⁺ (ES⁺) 525.3 (M − H)⁻ (ES⁻) |
| C35 | 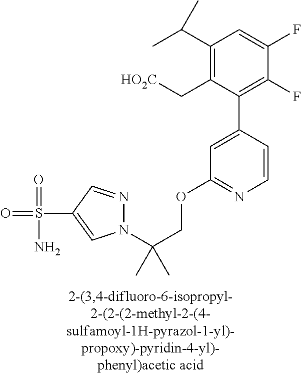 [SMILES: CC(C)c1cc(F)c(F)c(-c2ccnc(OCC(C)(C)n3cc(S(N)(=O)=O)cn3)c2)c1CC(=O)O] | A9 + B7 | ¹H NMR (DMSO-d₆) δ 8.27 (s, 1H), 8.25 (d, J = 5.2 Hz, 1H), 7.77 (s, 1H), 7.47 (dd, J = 12.2, 8.2 Hz, 1H), 7.21 (s, 2H), 6.92- 6.88 (m, 1H), 6.70 (s, 1H), 4.58 (d, J = 9.2 Hz, 2H), 3.39 (d, J = 6.7 Hz, 2H), 3.04-2.96 (m, 1H), 1.64 (s, 6H), 1.17 (d, J = 6.7 Hz, 6H). One exchangeable proton not observed. | m/z 509.0 (M + H)⁺ (ES⁺) 507.3 (M − H)⁻ (ES⁻) |
| C42 | 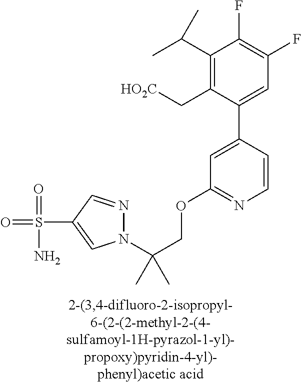 [SMILES: CC(C)c1c(F)c(F)cc(-c2ccnc(OCC(C)(C)n3cc(S(N)(=O)=O)cn3)c2)c1CC(=O)O] | A9 + B8 | ¹H NMR (DMSO-d₆) δ 8.26 (s, 1H), 8.19 (d, J = 5.2 Hz, 1H), 7.76 (s, 1H), 7.25-7.17 (m, 3H), 6.87 (dd, J = 5-2, 1.4 Hz, 1H), 6.65 (s, 1H), 4.57 (s, 2H), 3.48 (s, 2H), 3.14-3.03 (m, 1H), 1.64 (s, 6H), 1.30 (d, J = 6.9 Hz, 6H). One exchangeable proton not observed. | m/z 509.3 (M + H)⁺ (ES⁺) |

### Intermediate C11: 2-(2-(2-(3-(2-hydroxypropan-2-yl)-5-sulfamoylphenethoxy)-pyridin-4-yl)-6-isopropylphenyl)acetic acid

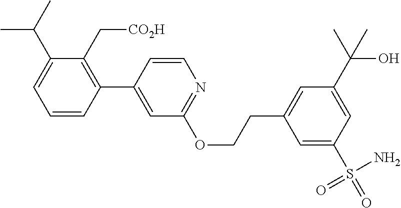 [SMILES: CC(C)c1cccc(-c2ccnc(OCCc3cc(C(C)(C)O)cc(S(N)(=O)=O)c3)c2)c1CC(=O)O]

### Step A: 2-(2-(2-(3-(N,N-bis(4-methoxybenzyl)sulfamoyl)-5-(2-hydroxypropan-2-yl)-phenethoxy)pyridin-4-yl)-6-isopropylphenyl)acetic acid

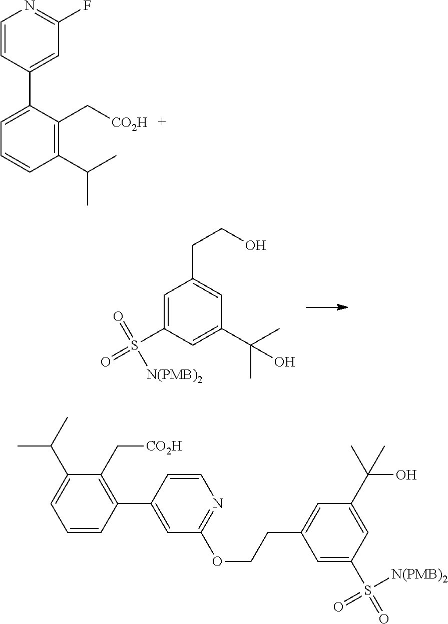

3-(2-hydroxyethyl)-5-(2-hydroxypropan-2-yl)-N,N-bis(4-methoxybenzyl)benzene-sulfonamide (Intermediate A7) (0.5 g, 1.00 mmol) and 2-(2-(2-fluoropyridin-4-yl)-6-isopropylphenyl)acetic acid (Intermediate B1) (0.274 g, 1.00 mmol) were dissolved in THF (20 mL) and cooled to 0° C. NaH (60% dispersion in mineral oil) (0.120 g, 3.00 mmol) was then added and the reaction was stirred for 10 min at this temperature before warming to RT and stirring for 5 h. The mixture was then diluted with EtOAc (50 mL) and acidified to pH <3 with 1 M HCl. The organic layer was separated and the aqueous layer re-extracted with EtOAc (2×50 mL). The combined organic layer was then dried (phase separator), and concentrated in vacuo. The crude product was purified by FC (0-100% EtOAc/isohexane) to afford the title compound (268 mg, 32%) as a pale yellow oil.

LCMS m/z 753.5 (M+H)⁺ (ES⁺).

¹H NMR (DMSO-d₆) δ 8.18 (d, J=5.2 Hz, 1H), 7.80-7.76 (m, 1H), 7.71-7.67 (m, 1H), 7.61-7.57 (m, 1H), 7.40 (dd, J=7.9, 1.4 Hz, 1H), 7.30 (t, J=7.7 Hz, 1H), 6.94 (dd, J=7-5, 1.4 Hz, 1H), 6.93-6.90 (m, 4H), 6.88 (dd, J=5.2, 1.5 Hz, 1H), 6.77-6.72 (m, 4H), 6.63 (s, 1H), 4.52 (t, J=6.7 Hz, 2H), 4.16 (s, 4H), 4.14-4.08 (m, 1H), 3.68 (s, 6H), 3.47 (s, 2H), 3.15 (t, J=6.7 Hz, 2H), 3.09-3.00 (m, 1H), 1.42 (s, 6H), 1.19 (d, J=6.8 Hz, 6H). One exchangeable proton not observed.

### Step B: 2-(2-(2-(3-(2-hydroxypropan-2-yl)-5-sulfamoylphenethoxy)pyridin-4-yl)-6-isopropylphenyl) acetic acid

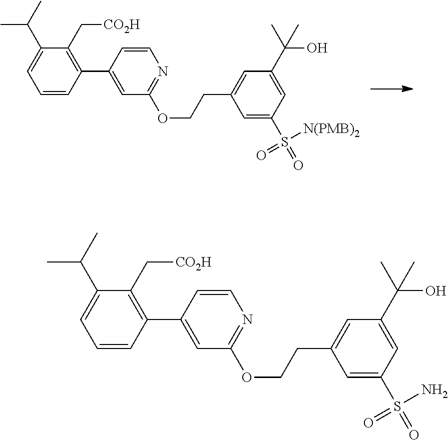

2-(2-(2-(3-(N,N-bis(4-methoxybenzyl)sulfamoyl)-5-(2-hydroxypropan-2-yl)-phenethoxy)pyridin-4-yl)-6-isopropylphenyl)acetic acid (268 mg, 0.356 mmol) was dissolved in MeCN (6 mL). A solution of CAN (976 mg, 1.780 mmol) in water (2 mL) was added portionwise over 5 min. The orange mixture was stirred for 2 h. H₂O (5 mL) and DCM (20 mL) was added and the organic phase was separated, washed with brine (10 mL), dried using a phase separator, and concentrated in vacuo to give a yellow oil. The crude product was purified by FC (0-10% MeOH/DCM) to afford the title compound (58 mg, 32%) as a clear colourless oil.

LCMS m/z 513.4 (M+H)⁺ (ES⁺).

¹H NMR (DMSO-d₆) δ 8.23-8.17 (m, 1H), 7.84 (s, 1H), 7.64 (d, J=1.9 Hz, 1H), 7.61 (d, J=1.7 Hz, 1H), 7.40 (d, J=8.2 Hz, 1H), 7.36-7.26 (m, 3H), 7.03 (d, J=7.1 Hz, 1H), 6.90 (d, J=5.2 Hz, 1H), 6.69 (s, 1H), 4.53 (t, J=6.8 Hz, 2H), 3.50 (s, 2H), 3.15 (q, J=7.0 Hz, 2H), 3.05 (s, 1H), 1.44 (s, 6H), 1.19 (d, J=6.7 Hz, 6H). Two exchangeable protons not observed.

### Intermediate C17: 2-(5-(2-(3-(2-hydroxypropan-2-yl)-5-sulfamoylphenethoxy)-pyridin-4-yl)-2,3-dihydro-1H-inden-4-yl)acetic acid

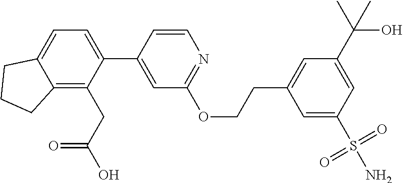 [SMILES: CC(C)(O)c1cc(CCOc2cc(-c3ccc4c(c3CC(=O)O)CCC4)ccn2)cc(S(N)(=O)=O)c1]

### Step A: tert-butyl 2-(5-(2-(3-(N,N-bis(4-methoxybenzyl)sulfamoyl)-5-(2-hydroxy-propan-2-yl)phenethoxy)pyridin-4-yl)-2,3-dihydro-1H-inden-4-yl)acetate

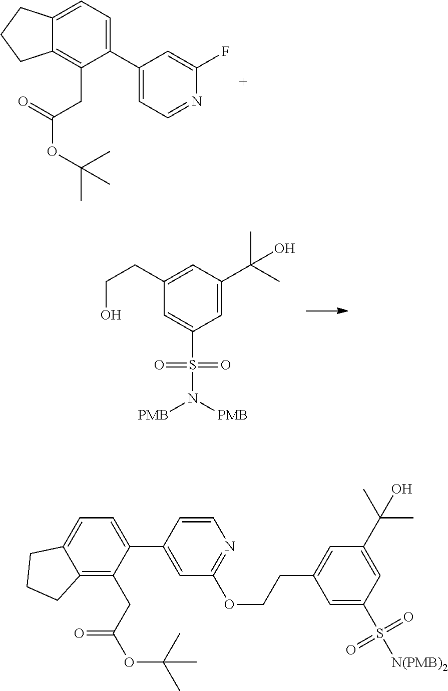

3-(2-hydroxyethyl)-5-(2-hydroxypropan-2-yl)-N,N-bis(4-methoxybenzyl)benzene-sulfonamide (Intermediate A7) (0.4 g, 0.801 mmol) and tert-butyl 2-(5-(2-fluoro-pyridin-4-yl)-2,3-dihydro-1H-inden-4-yl)acetate (Intermediate B3) (0.26 g, 0.801 mmol) were dissolved in THF (7 mL) and cooled to 0° C. Sodium hydride (60% dispersion in mineral oil) (0.096 g, 2.402 mmol) was then added and the reaction was stirred for 10 min at this temperature before warming to RT and stirring for 24 h. The mixture was then diluted EtOAc (50 mL), washed with brine (50 mL), dried (phase separator), and concentrated in vacuo. The crude product was purified by FC (0-100% EtOAc/isohexane) to afford the title compound (245 mg, 34.5%) as a pink oil.

LCMS m/z 807.7 (M+H)⁺ (ES⁺); 805.8 (M−H)⁻ (ES⁻).

¹H NMR (DMSO-d₆) δ 8.18 (d, J=5.5 Hz, 1H), 7.78 (t, J=1.7 Hz, 1H), 7.68 (t, J=1.7 Hz, 1H), 7.58 (t, J=1.7 Hz, 1H), 7.19 (d, J=7.7 Hz, 1H), 6.96-6.88 (m, 5H), 6.85 (dd, J=5.3, 1.4 Hz, 1H), 6.76-6.70 (m, 4H), 6.60-6.57 (m, 1H), 5.26 (s, 1H), 4.52 (t, J=6.6 Hz, 2H), 4.15 (s, 4H), 3.68 (s, 6H), 3.40 (s, 2H), 3.14 (t, J=6.6 Hz, 2H), 2.93 (t, J=7.4 Hz, 2H), 2.82 (t, J=7.4 Hz, 2H), 2.03 (p, J−7.5 Hz, 2H), 1.42 (s, 6H), 1.32 (s, 9H).

### Step B: 2-(5-(2-(3-(2-hydroxypropan-2-yl)-5-sulfamoylphenethoxy)pyridin-4-yl)-2,3-dihydro-1H-inden-4-yl)acetic acid

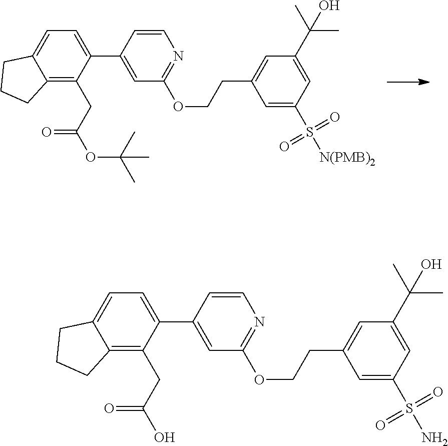

tert-Butyl 2-(5-(2-(3-(N,N-bis(4-methoxybenzyl)sulfamoyl)-5-(2-hydroxypropan-2-yl)-phenethoxy)pyridin-4-yl)-2,3-dihydro-1H-inden-4-yl)acetate (245 mg, 0.304 mmol) was dissolved in MeCN (6 mL). A solution of CAN (832 mg, 1.518 mmol) in water (2 mL) was added portion-wise over 5 min. The orange mixture was stirred for 2 h. Water (5 mL) and DCM (20 mL) were added and the organic phase was separated, washed with brine (10 mL), dried using a phase separator, and concentrated in vacuo to give a yellow oil. The crude product was purified by FC (0-100% EtOAc/isohexane) to afford tert-butyl 2-(5-(2-(3-(2-hydroxypropan-2-yl)-5-sulfamoylphenethoxy)pyridin-4-yl)-2,3-dihydro-1H-inden-4-yl)acetate (135 mg, 78%) as a clear colourless oil. This was dissolved in a 2:1 mixture of TFA and water (4 mL) and stirred at RT for 20 h. The reaction was concentrated in vacuo. The resulting residue was suspended in toluene (2 mL) then concentrated in vacuo (2×), to afford the title compound (120 mg, 72%) as a light brown solid.

LCMS m/z 511.5 (M+H)⁺ (ES⁺); 509.2 (M−H)⁻ (ES⁻).

¹H NMR (DMSO-d₆) δ 8.18 (d, J=5.2 Hz, 1H), 7.84 (t, J=1.9 Hz, 1H), 7.66-7.59 (m, 2H), 7.33-7.28 (m, 2H), 7.21 (d, J=7.6 Hz, 1H), 7.02 (d, J=7.7 Hz, 1H), 6.90 (dd, J=5.2, 1.5 Hz, 1H), 6.70 (s, 1H), 4.53 (t, J=6.8 Hz, 2H), 3.44 (s, 2H), 3.14 (t, J=6.8 Hz, 2H), 2.93 (t, J=7.4 Hz, 2H), 2.82 (t, J=7.4 Hz, 2H), 2.04 (p, J=7.5 Hz, 2H), 1.44 (s, 6H). Two exchangeable protons not observed.

### Intermediate C26: 2-(4-fluoro-2-(2-(4-fluoro-3-sulfamoylphenethoxy)pyridin-4-yl)-6-isopropylphenyl)acetic acid

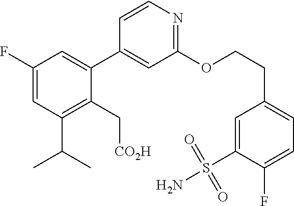 [SMILES: CC(C)c1cc(F)cc(-c2ccnc(OCCc3ccc(F)c(S(N)(=O)=O)c3)c2)c1CC(=O)O]

### Step A: methyl 2-(2-(2-(3-(N,N-bis(4-methoxybenzyl)sulfamoyl)-4-fluorophenethoxy)-pyridin-4-yl)-4-fluoro-6-isopropylphenyl)acetate

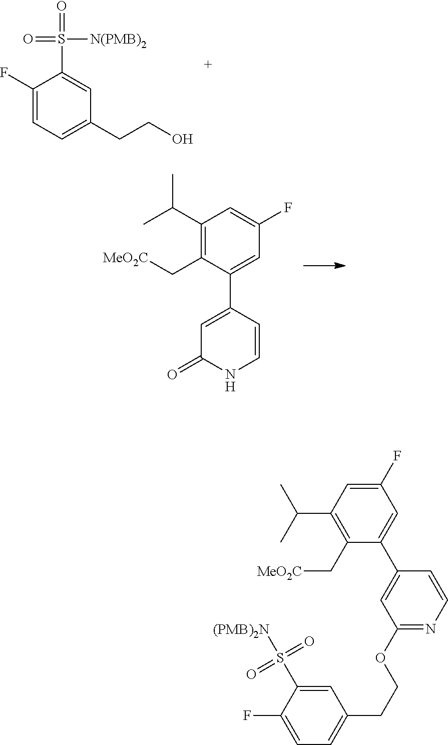

DIAD (300 μL, 1.543 mmol) was added to a mixture of 2-fluoro-5-(2-hydroxyethyl)-N,N-bis(4-methoxybenzyl)benzenesulfonamide (Intermediate A16) (540 mg, 1.18 mmol), methyl 2-(4-fluoro-2-isopropyl-6-(2-oxo-1,2-dihydropyridin-4-yl)phenyl)-acetate (Intermediate B5) (300 mg, 0.989 mmol) and PPh₃ (400 mg, 1.53 mmol) in THF (15 mL) at RT. After 7 h a further portion of PPh₃ (400 mg, 1.53 mmol) and DIAD (300 μL, 1.543 mmol) were added, stirred for 24 h then partitioned between TBME (80 mL) and water (50 mL), the organic layer separated, dried (MgSO₄), filtered and evaporated. The crude product was purified by FC (0-50% EtOAc/isohexane) to afford the title compound (554 mg, 45%) as a gum.

¹H NMR (CDCl₃) δ 8.15 (dd, J=5.2, 0.7 Hz, 1H), 7.86 (dd, J=6.8, 2.3 Hz, 1H), 7.52-7.47 (m, 1H), 7.12 (dd, J=9.9, 8.4 Hz, 1H), 7.06 (dd, J=10.2, 2.8 Hz, 1H), 7.00-6.95 (m, 4H), 6.81 (dd, J=5.2, 1.5 Hz, 1H), 6.78-6.73 (m, 4H), 6.71 (dd, J=8.5, 2.8 Hz, 1H), 6.64-6.62 (m, 1H), 4.58 (t, J=6.6 Hz, 2H), 4.34 (s, 4H), 3.78 (s, 6H), 3.66 (s, 3H), 3.54 (s, 2H), 3.14 (t, J=6.6 Hz, 2H), 3.08-3.00 (m, 1H), 1.24 (d, J=6.8 Hz, 6H).

### Step B: 2-(2-(2-(3-(N,N-bis(4-methoxybenzyl)sulfamoyl)-4-fluorophenethoxy)pyridin-4-yl)-4-fluoro-6-isopropylphenyl)acetic acid

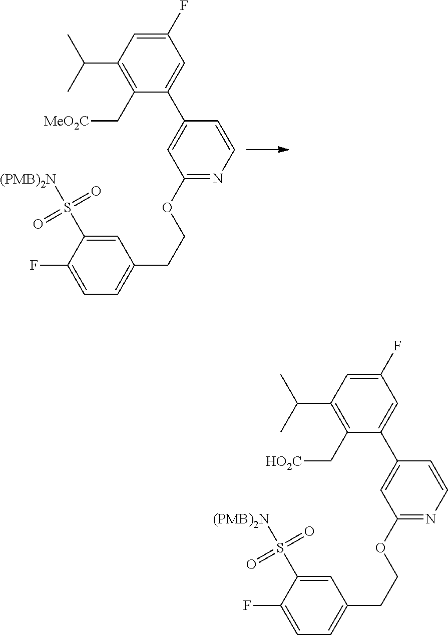

A mixture of methyl 2-(2-(2-(3-(N,N-bis(4-methoxybenzyl)sulfamoyl)-4-fluoro-phenethoxy)pyridin-4-yl)-4-fluoro-6-isopropylphenyl)acetate (700 mg, 0.564 mmol) in THF (5 mL), water (1 mL) and aq 1 M NaOH (800 μL, 0.800 mmol) was stirred at RT for 24 h. A further portion of aq 1 M NaOH (800 μL, 0.800 mmol) was added and the mixture heated at 50° C. for 24 h, cooled to RT, MeOH (1 mL) added and stirred for 4 days. After heating the solution at 50° C. for 6 h, the mixture was cooled, the organic solvent evaporated and the residue taken to pH 6 with aq 1 M HCl and extracted with TBME (2×15 mL). The organic layer was dried (MgSO₄), filtered, evaporated and the residue purified by FC (0-60% EtOAc/isohexane) to afford the title compound (345 mg, 67%) as a gum.

LCMS m/z 729.35 (M−H)⁻(ES⁻).

¹H NMR (DMSO-d₆) δ 12.46 (s, 1H), 8.20 (dd, J=5.2, 0.7 Hz, 1H), 7.72 (dd, J=7.0, 2.3 Hz, 1H), 7.68-7.63 (m, 1H), 7.36 (dd, J=10.5, 8.4 Hz, 1H), 7.23 (dd, J=10.5, 2.8 Hz, 1H), 6.99-6.93 (m, 4H), 6.87 (dd, J=5.2, 1.5 Hz, 1H), 6.81 (dd, J=8.9, 2.8 Hz, 1H), 6.79-6.73 (m, 4H), 6.64 (s, 1H), 4.52 (t, J=6.6 Hz, 2H), 4.26 (s, 4H), 3.69 (s, 6H), 3.43 (s, 2H), 3.11 (t, J=6.6 Hz, 2H), 3.02 (sept, J=6.5 Hz, 1H), 1.19-1.17 (d, J=6.5 Hz, 6H).

### Step C: 2-(4-fluoro-2-(2-(4-fluoro-3-sulfamoylphenethoxy)pyridin-4-yl)-6-isopropyl-phenyl)acetic acid

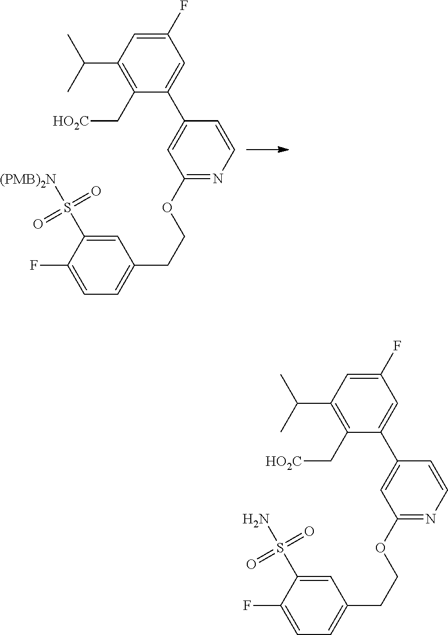

To a vial containing 2-(2-(2-(3-(N,N-bis(4-methoxybenzyl)sulfamoyl)-4-fluoro-phenethoxy)pyridin-4-yl)-4-fluoro-6-isopropylphenyl)acetic acid (300 mg, 0.39 mmol) was added TFA (5.0 mL). The reaction mixture was left to stir at RT for 2 h. The reaction mixture was concentrated in vacuo. The crude product was purified by FC (0-10% MeOH/DCM) to afford the title compound (194 mg, 96%) as a colourless solid.

LCMS m/z 491.2 (M+H)⁺ (ES⁺); 489.7 (M−H)⁻ (ES⁻).

¹H NMR (DMSO-d₆) δ 8.21 (d, J=5.2 Hz, 1H), 7.74 (dd, J=7.1, 2-3 Hz, 1H), 7.62 (s, 2H), 7.58 (ddd, J=7.6, 4.7, 2.4 Hz, 1H), 7.35 (dd, J=10.2, 8.4 Hz, 1H), 7.23 (dd, J=10.5, 2.8 Hz, 1H), 6.94-6.87 (m, 2H), 6.68 (s, 1H), 4.50 (t, J=6.7 Hz, 2H), 3.47 (s, 2H), 3.11 (t, J=6.7 Hz, 2H), 3.03 (p, J=6.5 Hz, 1H), 1.18 (d, J=6.7 Hz, 6H). One exchangeable proton not observed.

### Intermediate C39: 2-(2-(2-(2-(5-(dimethylcarbamoyl)-4-fluoro-3-sulfamoyl-1H-pyrazol-1-yl)-2-methylpropoxy)pyridin-4-yl)-4-fluoro-6-isopropylphenyl)acetic acid

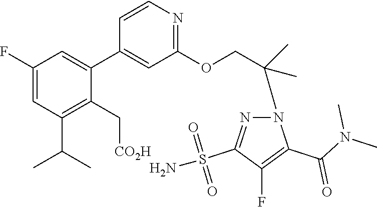 [SMILES: CC(C)c1cc(F)cc(-c2ccnc(OCC(C)(C)n3nc(S(N)(=O)=O)c(F)c3C(=O)N(C)C)c2)c1CC(=O)O]

### Step A: 3-(N,N-bis(4-methoxybenzyl)sulfamoyl)-1-(1-((4-(2-(2-(tert-butoxy)-2-oxoethyl)-5-fluoro-3-isopropylphenyl)pyridin-2-yl)oxy)-2-methylpropan-2-yl)-4-fluoro-1H-pyrazole-5-carboxylic acid

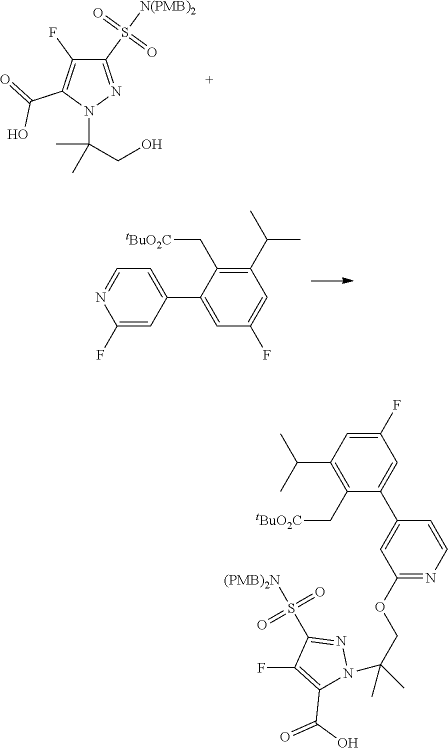

To a solution of 3-(N,N-bis(4-methoxybenzyl)sulfamoyl)-4-fluoro-1-(1-hydroxy-2-methylpropan-2-yl)-1H-pyrazole-5-carboxylic acid (Intermediate A25) (0.280 g, 537 μmol) in THF (6 mL) at 0° C. was added sodium hydride (60 wt % dispersion in mineral oil) (64.4 mg, 1.61 mmol) and the reaction left to stir for 15 min. tert-Butyl 2-(4-fluoro-2-(2-fluoropyridin-4-yl)-6-isopropylphenyl)acetate (Intermediate B4) (187 mg, 537 μmol) was then added to the reaction mixture, which was then warmed to RT and stirred for 16 h. The reaction was quenched with water and then the pH adjusted to ≤3 with 1 M aq HCl. The mixture was then extracted with EtOAc (2×20 mL), the organics passed through a phase separator and then concentrated in vacuo. The crude product was purified by FC (15-100% MeCN (0.1% formic acid)/water (0.1% formic acid)) to afford the title compound (0.135 g, 29%) as a white solid.

LCMS m/z 849.7 (M+H)⁺ (ES⁺).

¹H NMR (DMSO-d₆) δ 8.19 (d, J=5.2 Hz, 1H), 7.22 (dd, J=10.4, 2.7 Hz, 1H), 7.00 (d, J=8.5 Hz, 4H), 6.88 (dd, J=5.1, 1.5 Hz, 1H), 6.76 (m, 5H), 6.53 (s, 1H), 4.79 (s, 2H), 4.20 (s, 4H), 3.68 (s, 6H), 3.36 (s, 2H), 3.05-2.89 (m, 1H), 1.71 (s, 6H), 1.28 (d, J=1.3 Hz, 9H), 1.16 (d, J=6.6 Hz, 6H). One exchangeable proton not observed.

### Step B: 2-(2-(2-(2-(5-(dimethylcarbamoyl)-4-fluoro-3-sulfamoyl-1H-pyrazol-1-yl)-2-methylpropoxy)pyridin-4-yl)-4-fluoro-6-isopropylphenyl)acetic acid

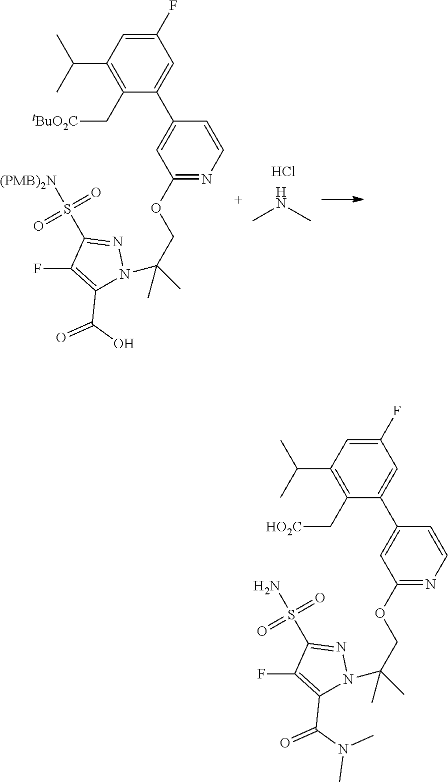

To a solution of 3-(N,N-bis(4-methoxybenzyl)sulfamoyl)-1-(1-((4-(2-(2-(tert-butoxy)-2-oxoethyl)-5-fluoro-3-isopropylphenyl)pyridin-2-yl)oxy)-2-methylpropan-2-yl)-4-fluoro-1H-pyrazole-5-carboxylic acid (0.234 g, 276 μmol) in DMF (3 mL) was added DIPEA (35.6 mg, 276 μmol) and HATU (115 mg, 303 μmol). The reaction was stirred at RT for 10 min and then dimethylamine hydrochloride (24.7 mg, 303 μmol) was added. The reaction was then heated at 80° C. for 65 h. The reaction was diluted with EtOAc (20 mL) and washed with water (50 mL) and then brine (3×50 mL). The organics were then passed through a phase separator and concentrated in vacuo. The crude product was purified by FC (0-100% EtOAc/isohexane). The isolated material was taken up in TFA (5 mL) and stirred for 16 h at RT. The reaction was then concentrated in vacuo and the resulting crude product was purified by FC (0-8% MeOH/DCM) to afford the title compound (0.09 g, 50%) as a pale yellow glass.

LCMS m/z 580.5 (M+H)⁺ (ES⁺).

¹H NMR (DMSO-d₆) δ 12.46 (s, 1H), 8.22 (d, J=5.2 Hz, 1H), 7.85 (s, 2H), 7.23 (dd, J=10.5, 2.8 Hz, 1H), 6.95-6.87 (m, 2H), 6.67 (d, J=1.4 Hz, 1H), 3.66-3.37 (m, 4H), 3.07-2.96 (m, 1H), 2.92 (s, 3H), 2.85 (s, 3H), 1.66 (s, 6H), 1.21-1.13 (m, 6H).

### Intermediate C40: 2-(2-(2-(2-(6-ethyl-3-sulfamoyl-4,5,6,7-tetrahydro-1H-pyrazolo-[3,4-c]pyridin-1-yl)-2-methylpropoxy)pyridin-4-yl)-4-fluoro-6-isopropylphenyl)acetic acid

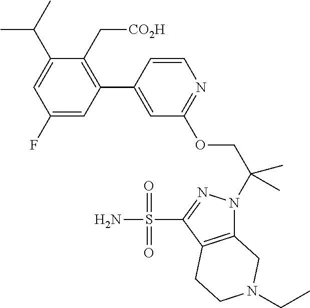 [SMILES: CCN1CCc2c(S(N)(=O)=O)nn(C(C)(C)COc3cc(-c4cc(F)cc(C(C)C)c4CC(=O)O)ccn3)c2C1]

### Step A: tert-butyl 2-(2-(2-(2-(3-(N,N-bis(4-methoxybenzyl)sulfamoyl)-4,5,6,7-tetrahydro-1H-pyrazolo[3,4-c]pyridin-1-yl)-2-methylpropoxy)pyridin-4-yl)-4-fluoro-6-isopropylphenyl)acetate

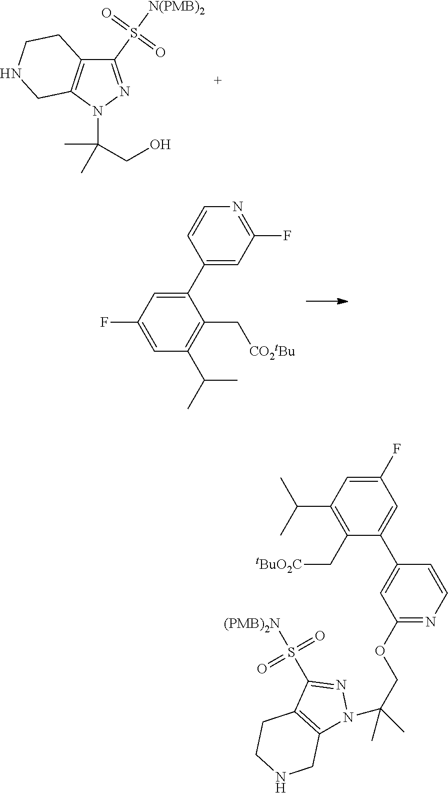

1-(1-hydroxy-2-methylpropan-2-yl)-N,N-bis(4-methoxybenzyl)-4,5,6,7-tetrahydro-1H-pyrazolo[3,4-c]pyridine-3-sulfonamide (375 mg, 729 μmol) (Intermediate A26) and tert-butyl 2-(4-fluoro-2-(2-fluoropyridin-4-yl)-6-isopropylphenyl)acetate (253 mg, 729 μmol) (Intermediate B4) were combined under N₂ then dissolved in THF (7 mL). The mixture was cooled to 0° C. then sodium hydride (60 wt % in mineral oil) (87 mg, 2.19 mmol) was added. The reaction was allowed to warm to RT and stirred for 16 h. The mixture was quenched with water (30 mL), transferred to a separating funnel and extracted with EtOAc (3×10 mL). The combined organic layers were dried over MgSO₄, filtered then concentrated in vacuo onto silica. The crude product was purified by FC (0-100% EtOAc/isohexane) then flushed with 10% MeOH (0.7 M NH₃) in DCM to afford the title compound (515 mg, 80%) as a flocculent white solid.

LCMS m/z 842.1 (M)+(ES⁺).

¹H NMR (DMSO-d₆) δ 8.19 (d, J=5.2 Hz, 1H), 7.23 (dd, J=10.5, 2.8 Hz, 1H), 6.96-6.90 (m, 4H), 6.88 (dd, J=5.2, 1.4 Hz, 1H), 6.80-6.71 (m, 5H), 6.61 (s, 1H), 4.55 (s, 2H), 4.13 (s, 4H), 4.04 (d, J=2.7 Hz, 2H), 3.68 (s, 6H), 3.39 (s, 2H), 3.00 (m, 1H), 2.74 (t, J=5.7 Hz, 2H), 1.64 (s, 6H), 1.31 (s, 9H), 1.17 (d, J=6.8 Hz, 6H). One CH₂ under DMSO peak, one exchangeable proton not observed.

### Step B: tert-butyl 2-(2-(2-(2-(3-(N,N-bis(4-methoxybenzyl)sulfamoyl)-6-ethyl-4,5,6,7-tetrahydro-1H-pyrazolo[3,4-c]pyridin-1-yl)-2-methylpropoxy)pyridin-4-yl)-4-fluoro-6-isopropylphenyl)acetate

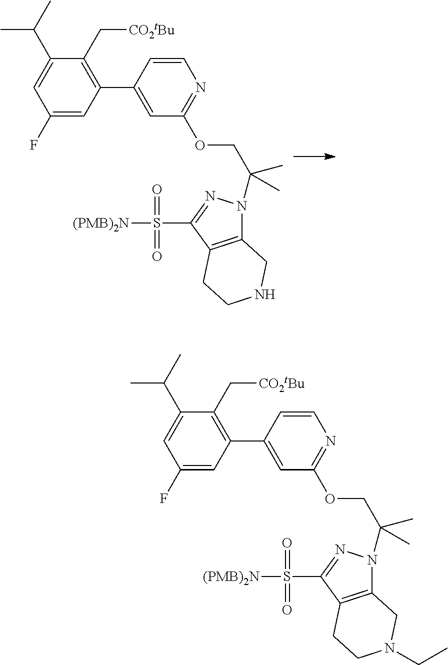

tert-Butyl 2-(2-(2-(2-(3-(N,N-bis(4-methoxybenzyl)sulfamoyl)-4,5,6,7-tetrahydro-1H-pyrazolo[3,4-c]pyridin-1-yl)-2-methylpropoxy)pyridin-4-yl)-4-fluoro-6-isopropyl-phenyl)acetate (515 mg, 612 μmol) and Pd/C (10 wt %) (65.1 mg, 61.2 μmol) were combined and purged by evacuating and back-filling with N₂ (×3). MeCN (4 mL) and EtOH (2 mL) were added then the mixture was placed under H₂ (5 bar) for 16 h. The mixture was filtered through a glass fibre filter disc and rinsed with EtOH (20 mL) followed by the removal of solvents in vacuo to afford the title compound (502 mg, 85%) as a white solid.

LCMS m/z 870.1 (M)+(ES⁺).

¹H NMR (DMSO-d₆) δ 8.19 (dd, J=5.3, 0.7 Hz, 1H), 7.24 (dd, J=10.5, 2.8 Hz, 1H), 6.97-6.92 (m, 4H), 6.89 (dd, J=5.2, 1.4 Hz, 1H), 6.80-6.71 (m, 5H), 6.59 (dd, J=1.5, 0.7 Hz, 1H), 4.59 (s, 2H), 4.14 (s, 4H), 3.79 (s, 2H), 3.68 (s, 6H), 3.40 (s, 2H), 3.01 (p, J=6.7 Hz, 1H), 2.61-2.51 (m, 6H), 1.67 (s, 6H), 1.31 (s, 9H), 1.18 (d, J=6.7 Hz, 6H), 1.05 (m, 3H).

### Step C: 2-(2-(2-(2-(6-ethyl-3-sulfamoyl-4,5,6,7-tetrahydro-1H-pyrazolo[3,4-c]-pyridin-1-yl)-2-methylpropoxy)pyridin-4-yl)-4-fluoro-6-isopropylphenyl)acetic acid

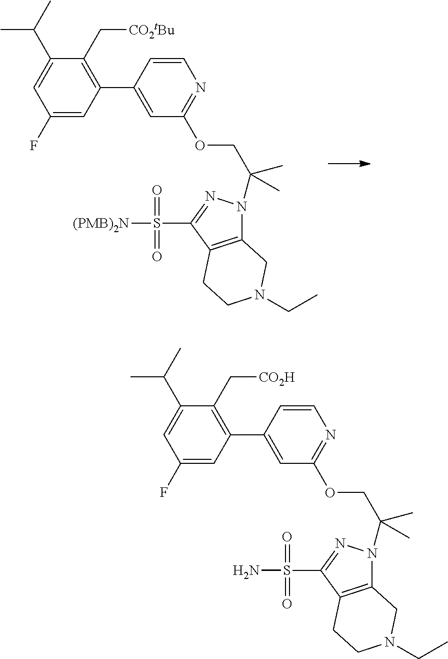

tert-Butyl 2-(2-(2-(2-(3-(N,N-bis(4-methoxybenzyl)sulfamoyl)-6-ethyl-4,5,6,7-tetrahydro-1H-pyrazolo[3,4-c]pyridin-1-yl)-2-methylpropoxy)pyridin-4-yl)-4-fluoro-6-isopropylphenyl)acetate (0.487 g, 560 μmol) was dissolved in TFA (5 mL) and stirred at RT for 6 h. The mixture was then concentrated to dryness, co-evaporated with toluene (10 mL) twice to dryness and purified by FC (0-10% (0.7 M ammonia/MeOH)/DCM) to afford the title compound (514 mg, 96%) as a sticky colourless foam.

LCMS m/z 574.5 (M+H)⁺ (ES⁺); 572.4 (M−H)⁻ (ES⁻).

¹H NMR (DMSO-d₆) δ 9.98 (s, 1H), 8.24-8.17 (m, 1H), 7.58 (s, 2H), 7.25 (dd, J=10.5, 2.8 Hz, 1H), 6.94 (dd, J=5.2, 1.4 Hz, 1H), 6.91 (dd, J=8.9, 2.8 Hz, 1H), 6.79-6.78 (m, 1H), 4.90 (d, J=14.9 Hz, 1H), 4.61-4.45 (m, 3H), 3.80-3.54 (m, 2H), 3.48 (s, 2H), 3.40-3.19 (m, 2H), 3.08-2.84 (m, 3H), 1.70 (d, J=4.0 Hz, 6H), 1.30 (t, J=7.3 Hz, 3H), 1.18 (d, J=6.8 Hz, 6H).

¹⁹F NMR (DMSO-d₆) δ −115.08 (t, J=9.8 Hz).

### Intermediate C41: 2-(2-(2-(2-(5-(dimethylcarbamoyl)-3-sulfamoyl-1H-pyrazol-1-yl)-2-methylpropoxy)pyridin-4-yl)-4-fluoro-6-isopropylphenyl)acetic acid

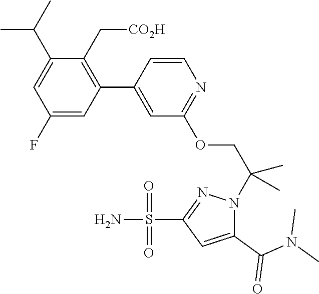 [SMILES: CC(C)c1cc(F)cc(-c2ccnc(OCC(C)(C)n3nc(S(N)(=O)=O)cc3C(=O)N(C)C)c2)c1CC(=O)O]

### Step A: 3-(N,N-bis(4-methoxybenzyl)sulfamoyl)-1-(1-((4-(2-(2-(tert-butoxy)-2-oxoethyl)-5-fluoro-3-isopropylphenyl)pyridin-2-yl)oxy)-2-methylpropan-2-yl)-1H-pyrazole-5-carboxylic acid, formic acid salt

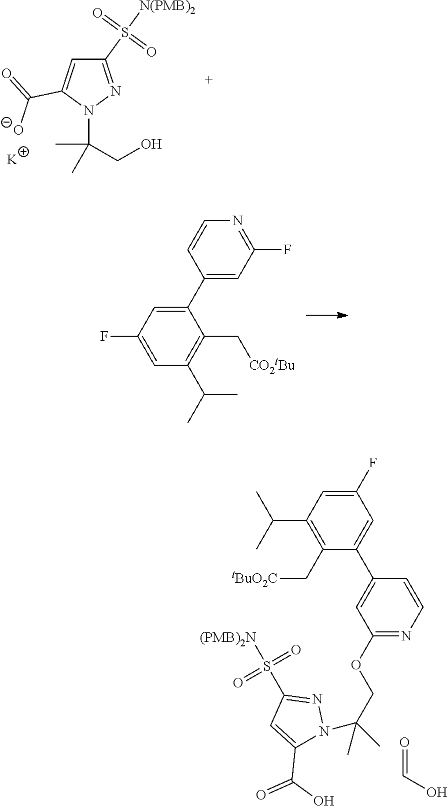

Prepared according to the general procedure of 3-(N,N-bis(4-methoxybenzyl)-sulfamoyl)-1-(1-((4-(2-(2-(tert-butoxy)-2-oxoethyl)-5-fluoro-3-isopropylphenyl)-pyridin-2-yl)oxy)-2-methylpropan-2-yl)-4-fluoro-1H-pyrazole-5-carboxylic acid (Intermediate C39, Step A) from potassium 3-(N,N-bis(4-methoxybenzyl)-sulfamoyl)-1-(1-hydroxy-2-methylpropan-2-yl)-1H-pyrazole-5-carboxylate (Intermediate A27) and tert-butyl 2-(4-fluoro-2-(2-fluoropyridin-4-yl)-6-isopropyl-phenyl)acetate (Intermediate B4) to afford the title compound (0.88 g, 41%) as a thick orange oil.

LCMS m/z 831.7 (M+H− formic acid)⁺ (ES⁺).

¹H NMR (DMSO-d₆) δ 14.19-12.49 (m, 1H), 8.18 (d, J=5.2 Hz, 1H), 8.13 (s, 1H), 7.22 (dd, J=10.5, 2.8 Hz, 1H), 7.05 (s, 1H), 6.99-6.91 (m, 4H), 6.87 (dd, J=5.2, 1.4 Hz, 1H), 6.77-6.70 (m, 5H), 6.51 (d, J=1.2 Hz, 1H), 4.85 (s, 2H), 4.14 (s, 4H), 3.67 (s, 6H), 3.37 (s, 2H), δ 3.04-2.94 (m, 1H), 1.77 (s, 6H), 1.29 (s, 9H), 1.17 (d, J=6.7 Hz, 6H). One exchangeable proton not observed.

### Step B: tert-butyl 2-(2-(2-(2-(3-(N,N-bis(4-methoxybenzyl)sulfamoyl)-5-(dimethyl-carbamoyl)-1H-pyrazol-1-yl)-2-methylpropoxy)pyridin-4-yl)-4-fluoro-6-isopropyl-phenyl)acetate

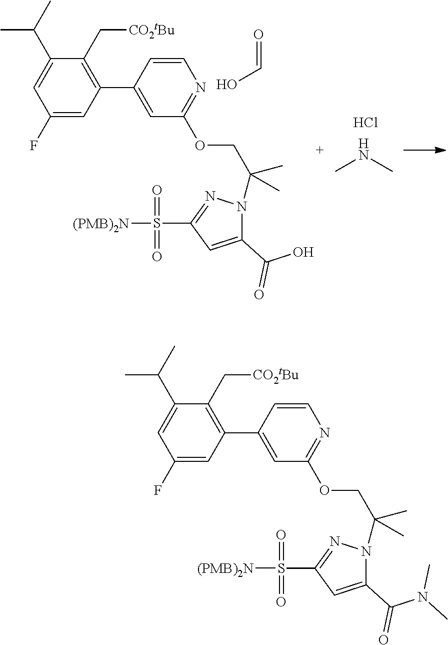

To a solution of 3-(N,N-bis(4-methoxybenzyl)sulfamoyl)-1-(1-((4-(2-(2-(tert-butoxy)-2-oxoethyl)-5-fluoro-3-isopropylphenyl)pyridin-2-yl)oxy)-2-methylpropan-2-yl)-1H-pyrazole-5-carboxylic acid, formic acid salt (0.88 g, 0.93 mmol) in DMF (3 mL) was added DIPEA (0.65 mL, 3.7 mmol), dimethylamine hydrochloride (84 mg, 1.0 mmol) and HATU (0.39 g, 1.0 mmol). The reaction was stirred at RT for 4 h. The reaction mixture was diluted with EtOAc (20 mL) and washed with water (50 mL) and then brine (3×50 mL). The organics were then passed through a phase separator and concentrated to dryness. The crude product was purified by FC (0-100% EtOAc/isohexane) to afford the title compound (0.60 g, 60%) as a pale yellow glass.

LCMS m/z 859.0 (M+H)⁺ (ES⁺).

¹H NMR (DMSO-d₆) δ 8.22 (d, J=5.2 Hz, 1H), 7.23 (dd, J=10.5, 2.8 Hz, 1H), 6.97-6.92 (m, 4H), 6.90 (dd, J=5.2, 1.4 Hz, 1H), 6.85 (s, 1H), 6.80-6.73 (m, 5H), 6.56 (s, 1H), 4.66 (s, 2H), 4.15 (s, 4H), 3.67 (s, 6H), 3.38 (s, 2H), 3.05-2.95 (m, 1H), 2.92 (s, 3H), 2.81 (s, 3H), 1.66 (s, 6H), 1.30 (s, 9H), 1.21-1.12 (m, 6H). 19F NMR (DMSO-d₆) δ −114.90 (t, J=9.5 Hz).

### Step C: 2-(2-(2-(2-(5-(dimethylcarbamoyl)-3-sulfamoyl-1H-pyrazol-1-yl)-2-methylpropoxy)pyridin-4-yl)-4-fluoro-6-isopropylphenyl)acetic acid

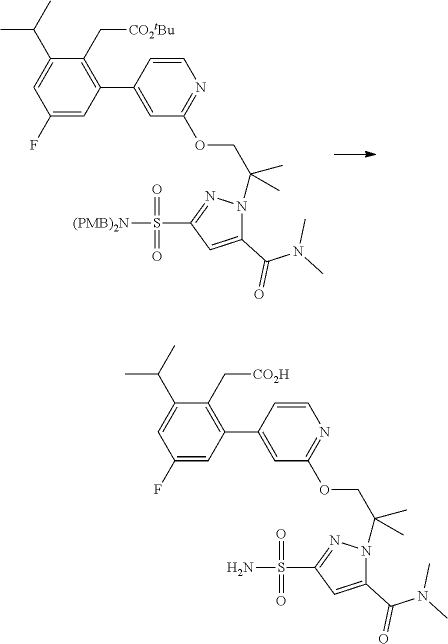

tert-Butyl 2-(2-(2-(2-(3-(N,N-bis(4-methoxybenzyl)sulfamoyl)-5-(dimethylcarbamoyl)-1H-pyrazol-1-yl)-2-methylpropoxy)pyridin-4-yl)-4-fluoro-6-isopropylphenyl)acetate (558 mg, 650 μmol) was dissolved in TFA (5 mL) and stirred at RT for 24 h. The reaction was concentrated in vacuo and the resulting residue purified by FC (0-10% MeOH/DCM) to afford the title compound (326 mg, 88%) as a clear colourless oil.

LCMS m/z 562.4 (M+H)⁺ (ES⁺); 560.3 (M−H)⁻ (ES⁻).

¹H NMR (DMSO-d₆) δ 12.47 (br s, 1H), 8.22 (d, J=5.2 Hz, 1H), 7.52 (s, 2H), 7.22 (dd, J=10.6, 2.8 Hz, 1H), 6.95-6.87 (m, 2H), 6.69 (s, 1H), 6.66 (s, 1H), 4.65 (s, 2H), 3.47 (s, 2H), 3.07-2.99 (m, 1H), 2.89 (s, 3H), 2.81 (s, 3H), 1.68 (s, 6H), 1.18 (d, J=6.8 Hz, 6H).

### Intermediate C43: 2-(2-isopropyl-6-(2-((5-(5-sulfamoyl-1H-pyrazol-1-yl)pentyl)-oxy)pyridin-4-yl)phenyl)acetic acid

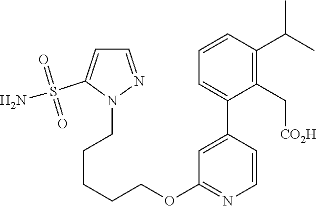 [SMILES: CC(C)c1cccc(-c2ccnc(OCCCCCn3nccc3S(N)(=O)=O)c2)c1CC(=O)O]

### Step A: 2-(2-(2-((5-(5-(N,N-bis(4-methoxybenzyl)sulfamoyl)-1H-pyrazol-1-yl)pentyl)-oxy)pyridin-4-yl)-6-isopropylphenyl)acetic acid

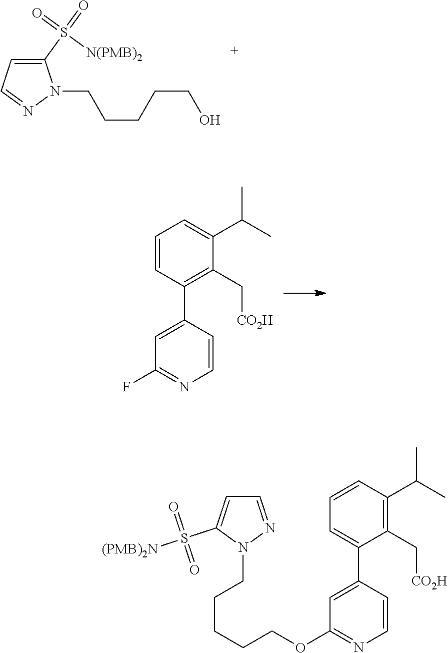

To a solution of 1-(5-hydroxypentyl)-N,N-bis(4-methoxybenzyl)-1H-pyrazole-5-sulfonamide (Intermediate A28) (500 mg, 1.06 mmol) and 2-(2-(2-fluoropyridin-4-yl)-6-isopropylphenyl)acetic acid (Intermediate B1) (288 mg, 1.06 mmol) in THF (6 mL) was added NaH (127 mg, 60 wt % in mineral oil, 3.17 mmol) at 0° C. Then the mixture was stirred at 40° C. for 12 h. The mixture was poured into MeOH (100 mL) slowly, then concentrated in vacuum. The residue was purified by reverse phase flash chromatography (water (0.1% TFA)-MeCN) to give the title compound (0.54 g, 70.8%) as a yellow oil.

LCMS: m/z 727.1 (M+H)⁺ (ES⁺).

¹H NMR (CDCl₃) δ 8.21 (d, 1H), 7.57 (d, 1H), 7.40-7.39 (m, 1H), 7.35 (t, 1H), 7.08 (d, 1H), 7.00 (d, 4H), 6.86-6.82 (m, 5H), 6.55 (d, 1H), 6.54 (d, 1H), 4.38-4.30 (m, 8H), 3.80 (s, 6H), 3.62 (s, 2H), 3.17-3.13 (m, 1H), 1.99-1.96 (m, 2H), 1.82-1.77 (m, 2H), 1.53-1.50 (m, 2H), 1.28 (d, 6H). One exchangeable proton not observed.

### Step B: 2-(2-isopropyl-6-(2-((5-(5-sulfamoyl-1H-pyrazol-1-yl)pentyl)oxy)pyridin-4-yl)-phenyl)acetic acid

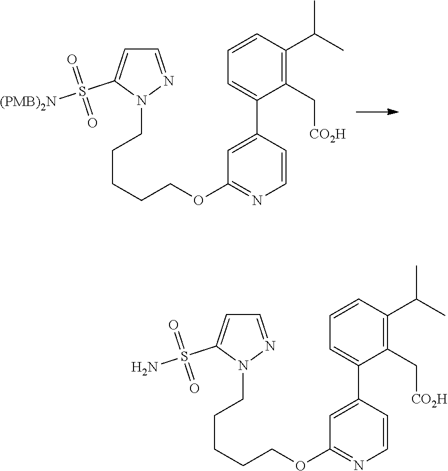

To a solution of 2-(2-(2-((5-(5-(N,N-bis(4-methoxybenzyl)sulfamoyl)-1H-pyrazol-1-yl)-pentyl)oxy)pyridin-4-yl)-6-isopropylphenyl)acetic acid (700 mg, 0.96 mmol) in DCM (5 mL) was added TFA (7.70 g, 67.5 mmol) at 20° C. Then the mixture was stirred at 20° C. for 12 h. The reaction mixture was concentrated in vacuum. The residue was triturated with MeOH (100 mL), filtered and the filtrate was concentrated in vacuum. The residue was purified by FC (PE:EtOAc, 3:1 to 1:1) to give the title compound (430 mg, 91.8%) as a yellow oil.

LCMS: m/z 487.1 (M+H)⁺ (ES⁺).

¹H NMR (CDCl₃) δ 8.12 (d, 1H), 7.44 (d, 1H), 7.32-7.29 (m, 2H), 7.01-6.99 (m, 1H), 6.79 (d, 1H), 6.66 (s, 2H), 5.79 (br s, 2H), 4.34 (t, 2H), 4.22 (t, 2H), 3.52 (s, 2H), 3.05-3.02 (m, 1H), 1.98-1.93 (m, 2H), 1.75-1.72 (m, 2H), 1.43-1.41 (m, 2H), 1.22 (d, 6H). One exchangeable proton not observed.

### Intermediate C44: 2-(2-isopropyl-6-(6-(2-(3-sulfamoyl-1H-pyrazol-1-yl)ethoxy)-pyridazin-4-yl)phenyl)acetic acid

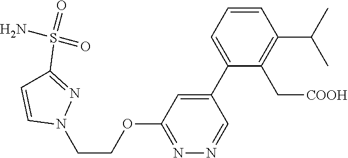 [SMILES: CC(C)c1cccc(-c2cnnc(OCCn3ccc(S(N)(=O)=O)n3)c2)c1CC(=O)O]

### Step A: 1-(2-((5-(2-bromo-3-isopropylphenyl)pyridazin-3-yl)oxy)ethyl)-N,N-bis(4-methoxybenzyl)-1H-pyrazole-3-sulfonamide

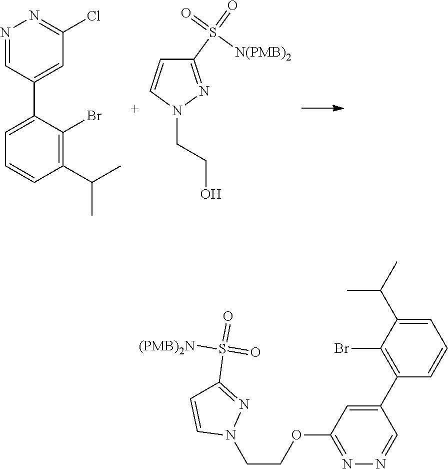

To a solution of 1-(2-hydroxyethyl)-N,N-bis(4-methoxybenzyl)-1H-pyrazole-3-sulfonamide (Intermediate A2) (640 mg, 1.48 mmol) in THF (10 mL) was added NaH (89 mg, 2.22 mmol, 60 wt % in mineral oil) at 0° C. The mixture was stirred at 0° C. for 0.5 h. Then to the above mixture was added 5-(2-bromo-3-isopropylphenyl)-3-chloropyridazine (Intermediate B9) (462 mg, 1.48 mmol) at 0° C. The resulting mixture was warmed to 20° C. and stirred at 20° C. for 15 h. The reaction mixture was quenched with H₂O (50 mL) and extracted with EtOAc (50 mL×3). The combined organic phases were washed with brine (50 mL×2), dried over anhydrous Na₂SO₄ and filtered. The filtrate was concentrated in vacuum. The residue was purified by FC (PE:EtOAc, 2:1 to 1:1) to give the title compound (850 mg, 81.1%) as a yellow oil.

LCMS: m/z 708.2 (M+H)⁺ (ES⁺).

¹H NMR (DMSO-d₆) δ 8.97 (d, 1H), 8.09 (d, 1H), 7.54-7.46 (m, 2H), 7.23-7.20 (m, 2H), 6.96 (m, 4H), 6.75 (m, 5H), 4.91 (t, 2H), 4.74 (t, 2H), 4.16 (s, 4H), 3.68 (s, 6H), 3.41-3.36 (m, 1H), 1.24 (d, 6H).

### Step B: (2-(tert-butoxy)-2-oxoethyl)zinc(II) bromide

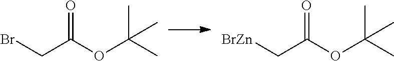

A mixture of zinc (3.35 g, 51.3 mmol) in 1 M HCl aqueous solution (50 mL) was stirred at 20° C. for 0.5 h. The mixture was filtered and the filter cake was dried to give activated zinc. To a mixture of activated zinc (2.5 g) and chloro(trimethyl)silane (139 mg, 1.28 mmol) in THF (30 mL) was added tert-butyl 2-bromoacetate (2.5 g, 12.82 mmol) dropwise at 50° C. under N₂. The mixture was stirred at 50° C. for 1 h. The reaction mixture was cooled to 20° C. to give the title compound (3.34 g, theoretical amount) in THF (30 mL) as a colourless liquid.

### Step C: tert-butyl 2-(2-(6-(2-(3-(N,N-bis(4-methoxybenzyl)sulfamoyl)-1H-pyrazol-1-yl)ethoxy)pyridazin-4-yl)-6-isopropylphenyl)acetate

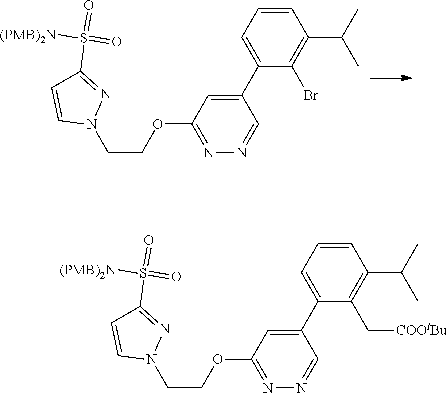

To a solution of 1-(2-((5-(2-bromo-3-isopropylphenyl)pyridazin-3-yl)oxy)ethyl)-N,N-bis(4-methoxybenzyl)-1H-pyrazole-3-sulfonamide (800 mg, 1.13 mmol) in THF (10 mL) at 25° C. was added the above THF solution of (2-(tert-butoxy)-2-oxoethyl)zinc(II) bromide (0.427 M, 13.3 mL), XPhos (54 mg, 113 μmol) and Pd₂(dba)₃ (52 mg, 56.6 μmol) under N₂. The reaction mixture was stirred at 70° C. for 2 h. The reaction mixture was quenched with H₂O (50 mL) and extracted with EtOAc (50 mL×3). The combined organic phases were washed with brine (50 ml×2), dried over anhydrous Na₂SO₄ and filtered. The filtrate was concentrated in vacuum. The residue was purified by FC (PE:EtOAc, 1:2 to 1:3) to give the title compound (700 mg, 83.3%) as a yellow oil.

LCMS: m/z 742.4 (M+H)⁺ (ES⁺).

¹H NMR (DMSO-d₆) δ 8.84 (d, 1H), 8.05 (d, 1H), 7.46 (d, 1H), 7.37 (t, 1H), 7.07-7.03 (m, 2H), 6.95 (d, 4H), 6.74 (d, 4H), 6.72 (d, 1H), 4.89 (t, 2H), 4.72 (t, 2H), 4.16 (s, 4H), 3.67 (s, 6H), 3.50 (s, 2H), 3.11-3.03 (m, 1H), 1.30 (s, 9H), 1.19 (d, 6H).

### Step D: 2-(2-isopropyl-6-(6-(2-(3-sulfamoyl-1H-pyrazol-1-yl)ethoxy)pyridazin-4-yl)-phenyl)acetic acid

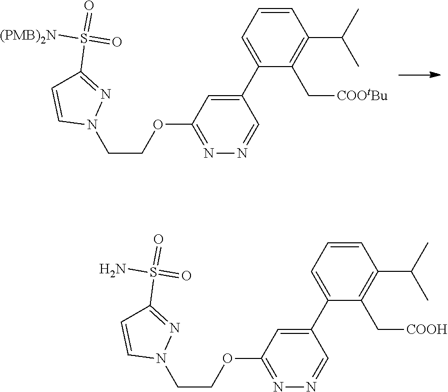

To a solution of tert-butyl 2-(2-(6-(2-(3-(N,N-bis(4-methoxybenzyl)sulfamoyl)-1H-pyrazol-1-yl)ethoxy)pyridazin-4-yl)-6-isopropylphenyl)acetate (700 mg, 944 μmol) in DCM (7 mL) was added TFA (7 mL). The mixture was stirred at 20° C. for 12 h. The reaction mixture was treated with MeOH (20 mL) and filtered. The filtrate was concentrated in vacuum. The residue was purified by FC (PE:EtOAc, 1:3 to 0:1 and then DCM:MeOH, 1:0 to 10:1) to give the title compound (300 mg, 71.4%) as a yellow oil.

LCMS: m/z 446.0 (M+H)⁺ (ES⁺).

¹H NMR (DMSO-d₆) δ 12.54 (br s, 1H), 8.85 (d, 1H), 7.96 (d, 1H), 7.48-7.35 (m, 4H), 7.12-7.08 (m, 2H), 6.60 (d, 1H), 4.87 (t, 2H), 4.67 (t, 2H), 2.53 (s, 2H), 3.12-3.04 (m, 1H), 1.19 (d, 6H).

### Intermediate C46: 2-(4-cyano-2-isopropyl-6-(2-(2-methyl-2-(4-sulfamoyl-1H-pyrazol-1-yl)propoxy)pyridin-4-yl)phenyl)acetic acid

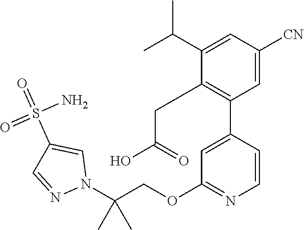 [SMILES: CC(C)c1cc(C#N)cc(-c2ccnc(OCC(C)(C)n3cc(S(N)(=O)=O)cn3)c2)c1CC(=O)O]

### Step A: tert-butyl 2-(2-(2-(2-(4-(N,N-bis(4-methoxybenzyl)sulfamoyl)-1H-pyrazol-1-yl)-2-methylpropoxy)pyridin-4-yl)-4-cyano-6-isopropylphenyl)acetate

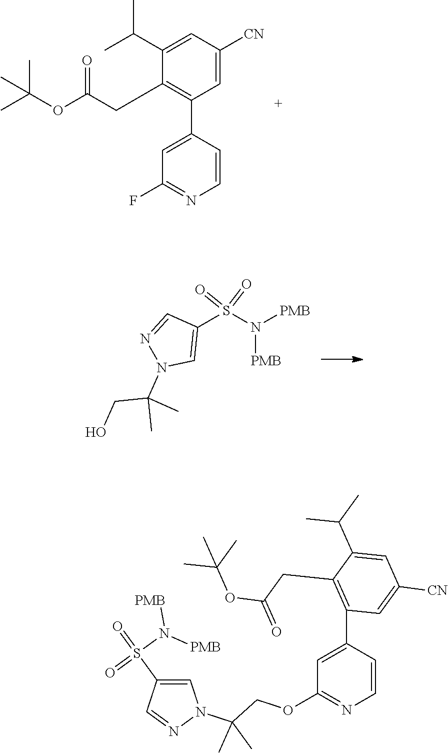

To a solution of tert-butyl 2-(4-cyano-2-(2-fluoropyridin-4-yl)-6-isopropylphenyl)-acetate (Intermediate B10) (0.5 g, 1.28 mmol, HCl salt) in THF (10 mL) was added NaH (169 mg, 4.23 mmol, 60% purity in mineral oil) and 1-(1-hydroxy-2-methylpropan-2-yl)-N,N-bis(4-methoxybenzyl)-1H-pyrazole-4-sulfonamide (Intermediate A9) (648 mg, 1.41 mmol). The mixture was stirred at 25° C. for 2 h under N₂, then quenched with sat aq NH₄Cl (30 mL) and extracted with EtOAc (50 mL×3). The organic layers were dried over anhydrous Na₂SO₄, filtered and concentrated in vacuum. The residue was purified by reverse phase flash chromatography (0.1% TFA) to give the title compound (0.8 g, 69.9% yield, 97.8% purity on LCMS) as a white solid.

LCMS: m/z 794.4 (M+H)⁺ (ES⁺).

¹H NMR (CDCl₃): δ 8.16 (d, 1H), 7.77 (s, 1H), 7.65 (s, 1H), 7.62 (d, 1H), 7.22 (d, 1H), 7.07 (d, 4H), 6.84-6.73 (m, 5H), 6.59 (s, 1H), 4.58 (s, 2H), 4.22 (s, 4H), 3.78 (s, 6H), 3.50 (s, 2H), 3.09-3.06 (m, 1H), 1.60 (s, 6H), 1.42 (s, 9H), 1.26 (d, 6H).

### Step B: 2-(4-cyano-2-isopropyl-6-(2-(2-methyl-2-(4-sulfamoyl-1H-pyrazol-1-yl)-propoxy)pyridin-4-yl)phenyl)acetic acid

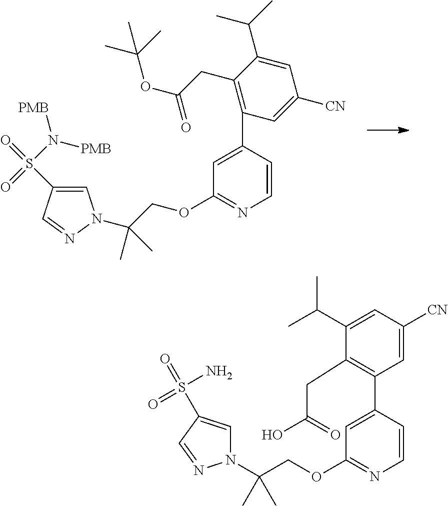

To a solution of tert-butyl 2-(2-(2-(2-(4-(N,N-bis(4-methoxybenzyl)sulfamoyl)-1H-pyrazol-1-yl)-2-methylpropoxy)pyridin-4-yl)-4-cyano-6-isopropylphenyl)acetate (1 g, 1.26 mmol) in DCM (1 mL) was added TFA (135 mmol, 10 mL). The mixture was stirred at 25° C. for 12 h. The reaction mixture was concentrated in vacuum. The residue was purified by reverse phase flash chromatography (0.1% TFA) to give the title compound (0.55 g, 87.8% yield, 100% purity on LCMS) as a white solid.

LCMS: m/z 498.3 (M+H)⁺ (ES⁺).

¹H NMR (CD₃OD): δ 8.35-8.24 (m, 2H), 7.86 (s, 1H), 7.78 (s, 1H), 7.54-7.51 (m, 1H), 7.38-7.29 (m, 2H), 4.68 (s, 2H), 3.71 (s, 2H), 3.25-3.20 (m, 1H), 1.77 (s, 6H), 1.30 (d, 6H). Three exchangeable protons not observed.

### Intermediate C47: 2-(4-(difluoromethoxy)-2-isopropyl-6-(2-(2-methyl-2-(4-sulfamoyl-1H-pyrazol-1-yl)propoxy)pyridin-4-yl)phenyl)acetic acid

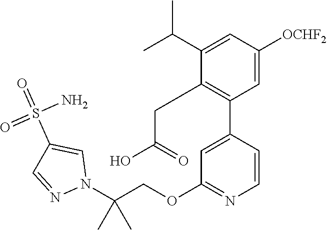 [SMILES: CC(C)c1cc(OC(F)F)cc(-c2ccnc(OCC(C)(C)n3cc(S(N)(=O)=O)cn3)c2)c1CC(=O)O]

### Step A: tert-butyl 2-(2-(2-(2-(4-(N,N-bis(4-methoxybenzyl)sulfamoyl)-1H-pyrazol-1-yl)-2-methylpropoxy)pyridin-4-yl)-4-(difluoromethoxy)-6-isopropylphenyl)acetate

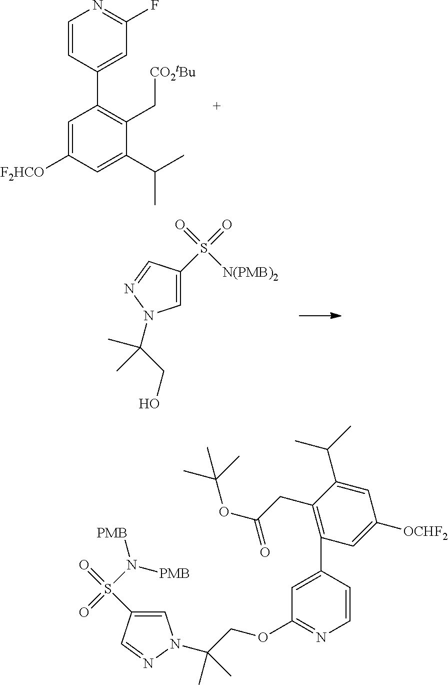

To a mixture of 1-(1-hydroxy-2-methylpropan-2-yl)-N,N-bis(4-methoxybenzyl)-1H-pyrazole-4-sulfonamide (Intermediate A9) (930 mg, 2.02 mmol) in THF (20 mL) was added NaH (202 mg, 5.06 mmol, 60% purity in mineral oil) in one portion at 25° C. under N₂. The mixture was stirred at 25° C. for 15 min, then tert-butyl 2-(4-(difluoro-methoxy)-2-(2-fluoropyridin-4-yl)-6-isopropylphenyl)acetate (Intermediate B11) (0.8 g, 2.02 mmol) in THF (20 mL) was added at 25° C. under N₂. The reaction mixture was stirred at 25° C. for 12 h, then quenched with 0.5 M aq HCl (30 mL) and extracted with EtOAc (30 mL×3). The combined organic phases were washed with brine (30 mL), dried over anhydrous Na₂SO₄, filtered and concentrated in vacuum. The residue was purified by FC (PE:EtOAc, 5:1 to 4:1) to give the title compound (1.3 g, 77.0% yield) as a yellow oil.

LCMS: m/z 835.3 (M+H)⁺ (ES⁺).

¹H NMR (CDCl₃): δ 8.13 (d, 1H), 7.75 (s, 1H), 7.64 (s, 1H), 7.08-7.05 (m, 5H), 6.83 (d, 1H), 6.79-6.75 (m, 5H), 6.61 (s, 1H), 6.49 (t, 1H), 4.56 (s, 2H), 4.21 (s, 4H), 3.76 (s, 6H), 3.24 (s, 2H), 3.09-3.03 (m, 1H), 1.69 (s, 6H), 1.39 (s, 9H), 1.23 (d, 6H).

### Step B: 2-(4-(difluoromethoxy)-2-isopropyl-6-(2-(2-methyl-2-(4-sulfamoyl-1H-pyrazol-1-yl)propoxy)pyridin-4-yl)phenyl)acetic acid

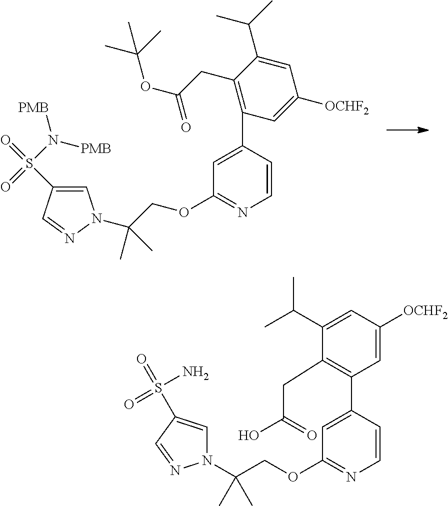

A mixture of tert-butyl 2-(2-(2-(2-(4-(N,N-bis(4-methoxybenzyl)sulfamoyl)-1H-pyrazol-1-yl)-2-methylpropoxy)pyridin-4-yl)-4-(difluoromethoxy)-6-isopropylphenyl)-acetate (1.3 g, 1.56 mmol) in DCM (7 mL) and TFA (94.5 mmol, 7 mL) was stirred at 25° C. for 12 h. The reaction mixture was concentrated in vacuum and the residue was treated with MeCN (3 mL), then filtered to obtain the filtrate. The filtrate was concentrated in vacuum and the residue was purified by reverse phase flash chromatography (water (0.1% TFA)-MeCN) to give the title compound (0.5 g, 59.6% yield) as a white solid.

LCMS: m/z 539.1 (M+H)⁺ (ES⁺).

### Intermediate C48: 2-(4-cyano-3-fluoro-2-isopropyl-6-(2-(2-methyl-2-(4-sulfamoyl-1H-pyrazol-1-yl)propoxy)pyridin-4-yl)phenyl)acetic acid

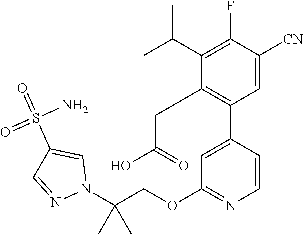 [SMILES: CC(C)c1c(F)c(C#N)cc(-c2ccnc(OCC(C)(C)n3cc(S(N)(=O)=O)cn3)c2)c1CC(=O)O]

### Step A: tert-butyl 2-(6-(2-(2-(4-(N,N-bis(4-methoxybenzyl)sulfamoyl)-1H-pyrazol-1-yl)-2-methylpropoxy)pyridin-4-yl)-4-cyano-3-fluoro-2-isopropylphenyl)acetate

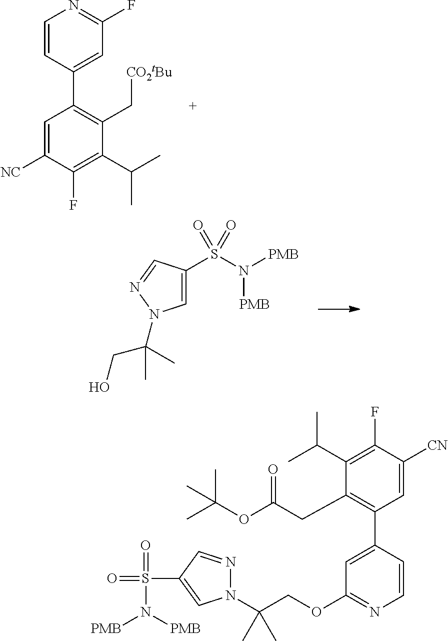

To a solution of 1-(1-hydroxy-2-methylpropan-2-yl)-N,N-bis(4-methoxybenzyl)-1H-pyrazole-4-sulfonamide (Intermediate A9) (583 mg, 1.27 mmol) in THF (30 mL) was added NaH (145 mg, 3.63 mmol, 60% purity in mineral oil) in portions at 0° C. The solution was stirred at 0° C. for 0.5 h, then tert-butyl 2-(4-cyano-3-fluoro-6-(2-fluoro-pyridin-4-yl)-2-isopropylphenyl)acetate (Intermediate B12) (450 mg, 1.21 mmol) was added and the resulting mixture was stirred at 25° C. for 2 h. The mixture was quenched with EtOH (10 mL) and the mixture was concentrated in vacuum. The residue was purified by reverse phase flash chromatography (water (0.1% TFA)-MeCN) to give the title compound (0.61 g, 54.5% yield, TFA salt) as a yellow solid.

LCMS: m/z 812.3 (M-TFA+H)⁺ (ES⁺).

¹H NMR (CDCl₃): δ 8.15 (d, 1H), 7.77 (s, 1H), 7.65 (s, 1H), 7.20 (d, 1H), 7.07 (d, 4H), 6.83-6.76 (m, 5H), 6.55 (s, 1H), 4.57 (s, 2H), 4.25 (s, 4H), 3.78 (s, 6H), 3.47 (s, 2H), 3.09-3.05 (m, 1H), 1.69 (s, 6H), 1.42 (s, 9H), 1.36 (d, 6H). TFA proton not observed.

### Step B: 2-(4-cyano-3-fluoro-2-isopropyl-6-(2-(2-methyl-2-(4-sulfamoyl-1H-pyrazol-1-yl)propoxy)pyridin-4-yl)phenyl)acetic acid

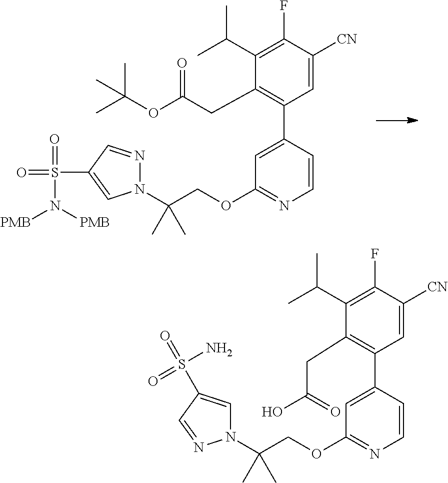

A solution of tert-butyl 2-(6-(2-(2-(4-(N,N-bis(4-methoxybenzyl)sulfamoyl)-1H-pyrazol-1-yl)-2-methylpropoxy)pyridin-4-yl)-4-cyano-3-fluoro-2-isopropylphenyl)-acetate (0.6 g, 648 μmol, TFA salt) in DCM (3 mL) and TFA (2.63 mL, 35.5 mmol) was stirred at 25° C. for 2 h. The reaction mixture was concentrated in vacuum and the residue was purified by reverse phase flash chromatography (water (0.1% HCl)-MeCN) to give the title compound (350 mg, 97.9% yield, HCl salt) as a yellow solid.

LCMS: m/z 516.2 (M−HCl+H)⁺ (ES⁺).

¹H NMR (CD₃OD): δ 8.29-8.26 (m, 2H), 7.79 (s, 1H), 7.59 (d, 1H), 7.19 (dd, 1H), 7.14 (s, 1H), 4.72 (s, 2H), 3.70 (s, 2H), 2.25-2.28 (m, 1H), 1.77 (s, 6H), 1.40 (dd, 6H). Three exchangeable protons and HCl proton not observed.

### Intermediate C40: 2-(4-(difluoromethoxy)-3-fluoro-2-isopropyl-6-(2-(2-methyl-2-(4-sulfamoyl-1H-pyrazol-1-yl)propoxy)pyridin-4-yl)phenyl)acetic acid

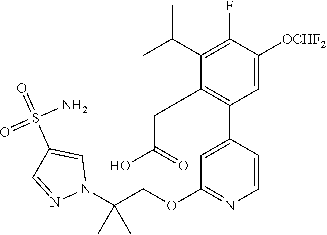 [SMILES: CC(C)c1c(F)c(OC(F)F)cc(-c2ccnc(OCC(C)(C)n3cc(S(N)(=O)=O)cn3)c2)c1CC(=O)O]

### Step A: tert-butyl 2-(6-(2-(2-(4-(N,N-bis(4-methoxybenzyl)sulfamoyl)-1H-pyrazol-1-yl)-2-methylpropoxy)pyridin-4-yl)-4-(difluoromethoxy)-3-fluoro-2-isopropylphenyl)-acetate

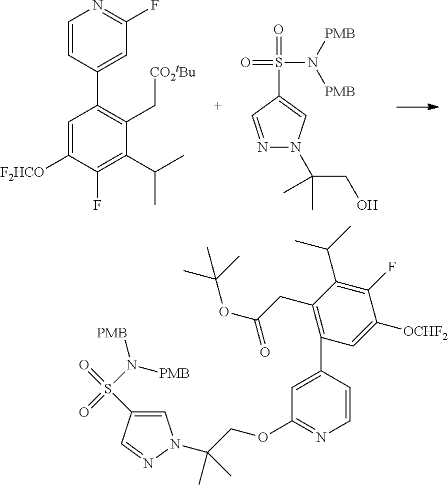

To a solution of tert-butyl 2-(4-(difluoromethoxy)-3-fluoro-6-(2-fluoropyridin-4-yl)-2-isopropylphenyl)acetate (Intermediate B13) (0.5 g, 1.21 mmol) in THF (10 mL) was added NaH (145 mg, 3.63 mmol, 60% purity in mineral oil) and 1-(1-hydroxy-2-methyl-propan-2-yl)-N,N-bis(4-methoxybenzyl)-1H-pyrazole-4-sulfonamide (Intermediate A9) (556 mg, 1.21 mmol). The mixture was stirred at 25° C. for 1 h, then quenched with MeOH (10 mL) and concentrated in vacuum. The residue was purified by reverse phase flash chromatography (0.1% TFA) to give the title compound (0.7 g, 67.5% yield, 99.5% purity on LCMS) as a yellow solid.

LCMS: m/z 853.3 (M+H)⁺ (ES⁺).

¹H NMR (CDCl₃): δ 8.15 (d, 1H), 7.74 (s, 1H), 7.65 (s, 1H), 7.08 (d, 4H), 6.91-6.84 (m, 2H), 6.77 (d, 4H), 6.63 (s, 1H), 6.52 (t, 1H), 4.54 (s, 2H), 4.22 (s, 4H), 3.77 (s, 6H), 3.41 (s, 2H), 3.10-3.00 (m, 1H), 2.45 (s, 6H), 1.43 (s, 9H), 1.37 (d, 6H).

### Step B: 2-(4-(difluoromethoxy)-3-fluoro-2-isopropyl-6-(2-(2-methyl-2-(4-sulfamoyl-1H-pyrazol-1-yl)propoxy)pyridin-4-yl)phenyl)acetic acid

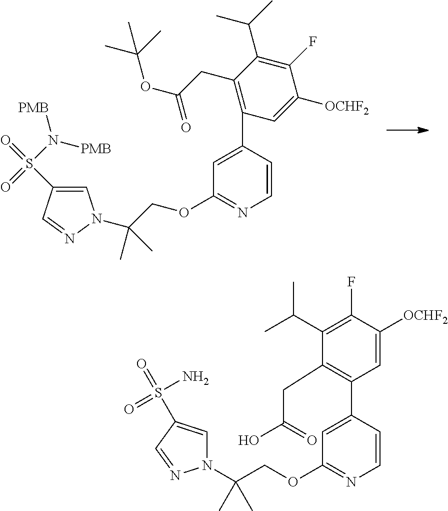

To a solution of tert-butyl 2-(6-(2-(2-(4-(N,N-bis(4-methoxybenzyl)sulfamoyl)-1H-pyrazol-1-yl)-2-methylpropoxy)pyridin-4-yl)-4-(difluoromethoxy)-3-fluoro-2-isopropylphenyl)acetate (0.7 g, 821 μmol) in DCM (7 mL) was added TFA (94.5 mmol, 7 mL). The mixture was stirred at 25° C. for 2 h, then concentrated in vacuum. The residue was purified by reverse phase flash chromatography (0.1% TFA) to give the title compound (0.4 g, 72.2% yield, 99.3% purity on LCMS, TFA salt) as a white solid.

LCMS: m/z 557.4 (M−TFA+H)⁺ (ES⁺).

¹H NMR (CD₃OD): δ 8.24 (s, 1H), 8.16 (d, 1H), 7.80 (s, 1H), 7.07-7.00 (m, 1H), 6.95-6.93 (m, 1H), 6.76-6.74 (m, 2H), 4.66 (s, 2H), 3.55 (s, 2H), 3.20-3.12 (m, 1H), 1.75 (s, 6H), 1.39 (d, 6H). Three exchangeable protons and TFA proton not observed.

### Intermediate C50: 2-(2-(2-(2-(4-(dimethylcarbamoyl)-3-sulfamoyl-1H-pyrazol-1-yl)-2-methylpropoxy)pyridin-4-yl)-4-fluoro-6-isopropylphenyl)acetic acid

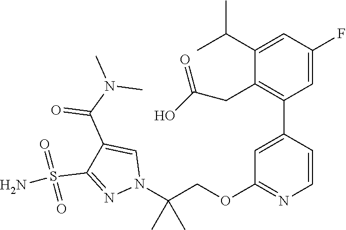 [SMILES: CC(C)c1cc(F)cc(-c2ccnc(OCC(C)(C)n3cc(C(=O)N(C)C)c(S(N)(=O)=O)n3)c2)c1CC(=O)O]

### Step A: 2-(2-(2-(2-(3-(N,N-bis(4-methoxybenzyl)sulfamoyl)-4-(dimethylcarbamoyl)-1H-pyrazol-1-yl)-2-methylpropoxy)pyridin-4-yl)-4-fluoro-6-isopropylphenyl)acetic

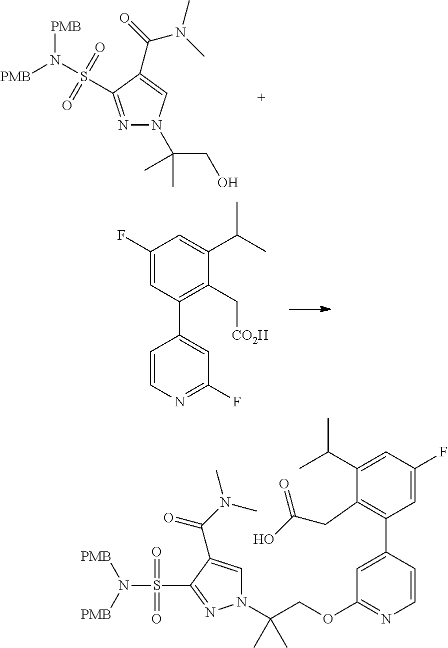

To a solution of 3-(N,N-bis(4-methoxybenzyl)sulfamoyl)-1-(1-hydroxy-2-methyl-propan-2-yl)-N,N-dimethyl-1H-pyrazole-4-carboxamide (Intermediate A29) (0.5 g, 942 μmol) in THF (30 mL) was added NaH (113 mg, 2.83 mmol, 60% purity in mineral oil) in portions at 0° C. under N₂. Then 2-(4-fluoro-2-(2-fluoropyridin-4-yl)-6-isopropylphenyl)acetic acid (Intermediate B14) (382 mg, 942 μmol, TFA salt) was added and the resulting mixture was stirred at 40° C. for 6 h. The mixture was quenched with EtOH (30 mL) at 0° C., then concentrated in vacuum. The residue was purified by reverse phase flash chromatography (water (0.05% HCl)-MeCN) to give the title compound (0.57 g, 72.2% yield, HCl salt) as a white solid.

LCMS: m/z 802.1 (M−HCl+H)⁺ (ES⁺).

¹H NMR (CDCl₃): δ 8.13 (d, 1H), 7.85 (s, 1H), 7.00-6.97 (m, 5H), 6.86-6.81 (m, 2H), 6.81-6.67 (m, 5H), 4.44 (s, 2H), 4.23 (s, 4H), 3.73 (s, 6H), 3.23 (s, 2H), 3.13-3.10 (m, 1H), 3.01 (s, 3H), 2.98 (s, 3H), 1.69 (s, 6H), 1.15 (d, 6H). One exchangeable proton and HCl proton not observed.

### Step B: 2-(2-(2-(2-(4-(dimethylcarbamoyl)-3-sulfamoyl-1H-pyrazol-1-yl)-2-methylpropoxy)pyridin-4-yl)-4-fluoro-6-isopropylphenyl)acetic acid

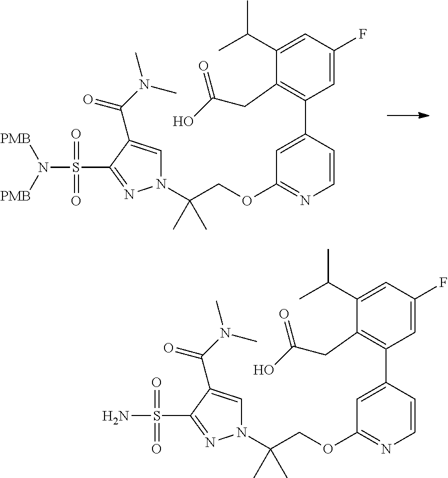

A solution of 2-(2-(2-(2-(3-(N,N-bis(4-methoxybenzyl)sulfamoyl)-4-(dimethyl-carbamoyl)-1H-pyrazol-1-yl)-2-methylpropoxy)pyridin-4-yl)-4-fluoro-6-isopropyl-phenyl)acetic acid (0.55 g, 656 μmol, HCl salt) in TFA (3 mL) and DCM (3 mL) was stirred at 25° C. for 12 h. The solution was concentrated in vacuum and the residue was purified by reverse phase flash chromatography (water (0.1% TFA)-MeCN) to give the title compound (345 mg, 77.8% yield, TFA salt) as a white solid.

LCMS: m/z 562.2 (M-TFA+H)⁺ (ES⁺).

¹H NMR (CDCl₃): δ 8.22 (d, 1H), 7.74 (s, 1H), 7.08 (dd, 1H), 6.91 (d, 1H), 6.74 (dd, 1H), 6.08 (s, 1H), 5.67 (br s, 2H), 4.43 (s, 2H), 3.33 (s, 2H), 3.13-3.05 (m, 7H), 1.80 (s, 6H), 1.24 (d, 6H). One exchangeable proton and TFA proton not observed.

### Intermediate C51: 2-(2-isopropyl-4-(methoxymethyl)-6-(2-(2-methyl-2-(4-sulfamoyl-1H-pyrazol-1-yl)propoxy)pyridin-4-yl)phenyl)acetic acid

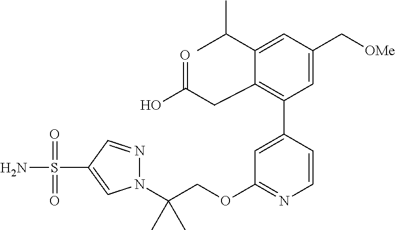 [SMILES: COCc1cc(-c2ccnc(OCC(C)(C)n3cc(S(N)(=O)=O)cn3)c2)c(CC(=O)O)c(C(C)C)c1]

### Step A: tert-butyl 2-(2-(2-(2-(4-(N,N-bis(4-methoxybenzyl)sulfamoyl)-1H-pyrazol-1-yl)-2-methylpropoxy)pyridin-4-yl)-6-isopropyl-4-(methoxymethyl)phenyl)acetate

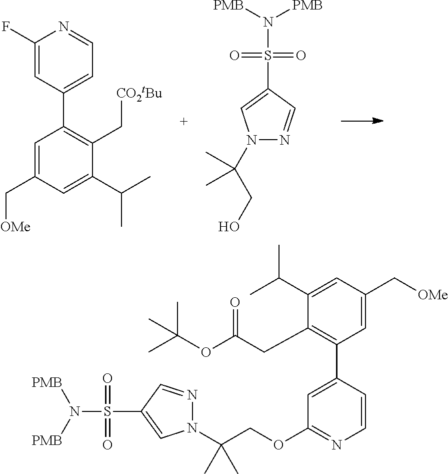

To a solution of tert-butyl 2-(2-(2-fluoropyridin-4-yl)-6-isopropyl-4-(methoxymethyl)-phenyl)acetate (Intermediate B15) (0.5 g, 1.34 mmol) in THF (10 mL) was added NaH (161 mg, 4.02 mmol, 60% purity in mineral oil) and 1-(1-hydroxy-2-methyl-propan-2-yl)-N,N-bis(4-methoxybenzyl)-1H-pyrazole-4-sulfonamide (Intermediate A9) (615 mg, 1.34 mmol). The mixture was stirred at 25° C. for 12 h under N₂, then quenched with sat aq NH₄Cl (10 mL) and extracted with DCM (20 mL×3). The organic layers were dried over anhydrous Na₂SO₄, filtered and concentrated in vacuum. The residue was purified by reverse phase flash chromatography (water (0.1% TFA)-MeCN) to give the title compound (0.8 g, 72.8% yield, 99% purity on LCMS) as a yellow solid.

LCMS: m/z 813.6 (M+H)⁺ (ES⁺).

¹H NMR (CD₃OD): δ 8.14 (d, 1H), 8.05 (s, 1H), 7.68 (s, 1H), 7.38 (d, 1H), 7.00 (dd, 4H), 6.92 (s, 1H), 6.90 (d, 1H), 6.72 (dd, 4H), 6.65 (s, 1H), 4.57 (s, 2H), 4.42 (s, 2H), 4.17 (s, 4H), 3.74 (s, 6H), 3.49 (s, 2H), 3.37 (s, 3H), 3.14-3.06 (m, 1H), 1.73 (s, 6H), 1.39 (s, 9H), 1.26 (d, 6H).

### Step B: 2-(2-isopropyl-4-(methoxymethyl)-6-(2-(2-methyl-2-(4-sulfamoyl-1H-pyrazol-1-yl)propoxy)pyridin-4-yl)phenyl)acetic acid

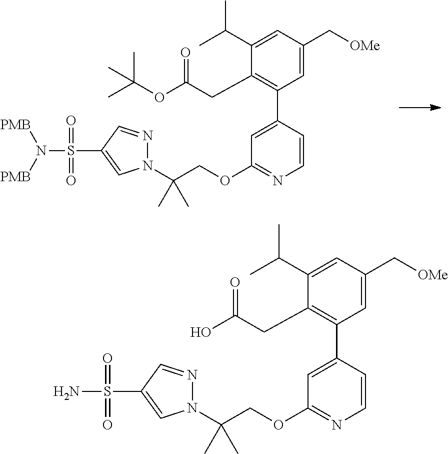

To a solution of tert-butyl 2-(2-(2-(2-(4-(N,N-bis(4-methoxybenzyl)sulfamoyl)-1H-pyrazol-1-yl)-2-methylpropoxy)pyridin-4-yl)-6-isopropyl-4-(methoxymethyl)phenyl)-acetate (0.8 g, 984 μmol) in DCM (10 mL) was added TFA (10 mL, 135 mmol). The mixture was stirred at 25° C. for 12 h, then concentrated in vacuum. The residue was purified by reverse phase flash chromatography (water (0.1% TFA)-MeCN) to give the title compound (0.4 g, 77.9% yield, 99% purity on LCMS) as a white solid.

LCMS: m/z 517.3 (M+H)⁺ (ES⁺).

¹H NMR (CD₃OD): δ 8.23 (s, 1H), 8.15 (d, 1H), 7.79 (s, 1H), 7.40 (d, 1H), 7.02 (s, 1H), 7.01 (d, 1H), 6.85 (s, 1H), 4.62 (s, 2H), 4.47 (s, 2H), 3.58 (s, 2H), 3.40 (s, 3H), 3.15-3.06 (m, 1H), 1.74 (s, 6H), 1.26 (d, 6H). Three exchangeable protons not observed.

### Intermediate C52: 2-(3-fluoro-2-isopropyl-4-(methoxymethyl)-6-(2-(2-methyl-2-(4-sulfamoyl-1H-pyrazol-1-yl)propoxy)pyridin-4-yl)phenyl)acetic acid

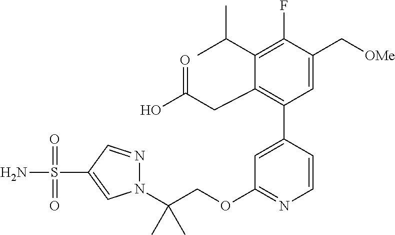 [SMILES: COCc1cc(-c2ccnc(OCC(C)(C)n3cc(S(N)(=O)=O)cn3)c2)c(CC(=O)O)c(C(C)C)c1F]

### Step A: tert-butyl 2-(6-(2-(2-(4-(N,N-bis(4-methoxybenzyl)sulfamoyl)-1H-pyrazol-1-yl)-2-methylpropoxy)pyridin-4-yl)-3-fluoro-2-isopropyl-4-(methoxymethyl)phenyl)-acetate

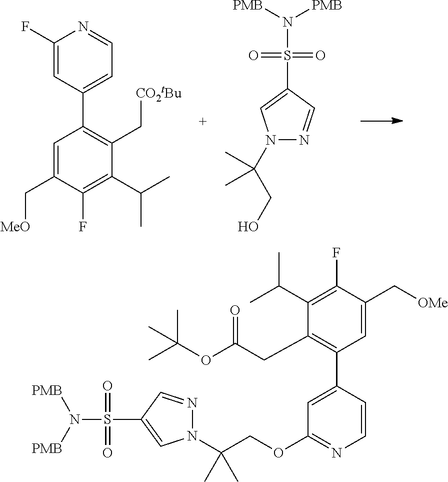

To a mixture of 1-(1-hydroxy-2-methylpropan-2-yl)-N,N-bis(4-methoxybenzyl)-1H-pyrazole-4-sulfonamide (Intermediate A9) (564 mg, 1.23 mmol) in THF (2 mL) was added NaH (123 mg, 3.07 mmol, 60% purity in mineral oil) at 25° C. under N₂. The reaction mixture was stirred at 25° C. for 15 min, then tert-butyl 2-(3-fluoro-6-(2-fluoropyridin-4-yl)-2-isopropyl-4-(methoxymethyl)phenyl)acetate (Intermediate B16) (0.48 g, 1.23 mmol) was added at 25° C. under N₂. The resulting reaction mixture was stirred at 25° C. for 12 h, then quenched with 0.5 M aq HCl (10 mL) and extracted with EtOAc (10 mL×3). The combined organic phases were washed with brine (10 mL), dried over anhydrous Na₂SO₄, filtered and concentrated in vacuum. The residue was purified by reverse phase flash chromatography (water (0.1% TFA)-MeCN) to give the title compound (0.8 g, 78.5% yield) as a white solid.

LCMS: m/z 832.6 (M+H)⁺ (ES⁺).

¹H NMR (CDCl₃): δ 8.17 (s, 1H), 7.72 (s, 1H), 7.64 (s, 1H), 7.20-7.10 (m, 5H), 7.00-6.97 (m, 1H), 6.79-6.75 (m, 5H), 4.60 (d, 2H), 4.50 (s, 2H), 4.25 (s, 4H), 3.75 (s, 6H), 3.44 (s, 2H), 3.42 (s, 3H), 3.08-3.03 (m, 1H), 1.70 (s, 6H), 1.45 (s, 9H), 1.35 (d, 6H).

### Step B: 2-(3-fluoro-2-isopropyl-4-(methoxymethyl)-6-(2-(2-methyl-2-(4-sulfamoyl-1H-pyrazol-1-yl)propoxy)pyridin-4-yl)phenyl)acetic acid

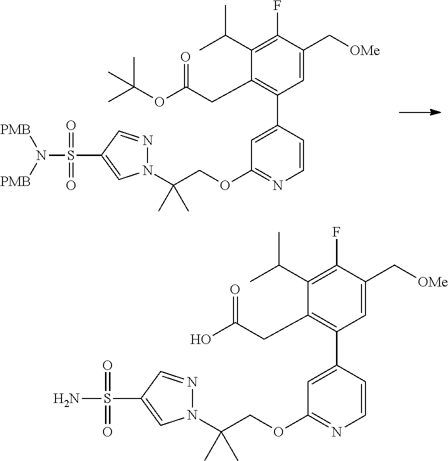

To a solution of tert-butyl 2-(6-(2-(2-(4-(N,N-bis(4-methoxybenzyl)sulfamoyl)-1H-pyrazol-1-yl)-2-methylpropoxy)pyridin-4-yl)-3-fluoro-2-isopropyl-4-(methoxymethyl)-phenyl)acetate (0.7 g, 842 μmol) in DCM (6 mL) was added TFA (6 mL, 81.0 mmol). The mixture was stirred at 25° C. for 12 h. The reaction mixture was concentrated in vacuum and the residue was treated with MeCN (5 mL) and filtered. The filtrate was concentrated in vacuum. The residue was purified by reverse phase flash chromatography (water (0.1% TFA)-MeCN) to give the title compound (0.32 g, 71.1% yield) as a white solid.

LCMS: m/z 535.4 (M+H)⁺ (ES⁺).

¹H NMR (CDCl₃): δ 8.26 (d, 1H), 8.17 (s, 1H), 7.78 (s, 1H), 7.15-7.10 (m, 2H), 6.88 (s, 1H), 4.55 (s, 2H), 4.51 (s, 2H), 3.53 (s, 2H), 3.46 (s, 3H), 3.08-3.03 (m, 1H), 1.70 (s, 6H), 1.40 (d, 6H). Three exchangeable protons not observed.

### Intermediate C53: 2-(4-fluoro-2-(2-(2-(4-fluoro-5-((4-methylpiperazin-1-yl)-methyl)-3-sulfamoyl-1H-pyrazol-1-yl)-2-methylpropoxy)pyridin-4-yl)-6-isopropyl-phenyl)acetic acid

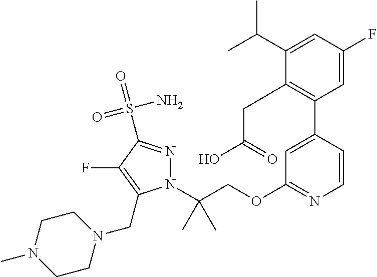 [SMILES: CC(C)c1cc(F)cc(-c2ccnc(OCC(C)(C)n3nc(S(N)(=O)=O)c(F)c3CN3CCN(C)CC3)c2)c1CC(=O)O]

### Step A: 2-(2-(2-(2-(3-(N,N-bis(4-methoxybenzyl)sulfamoyl)-4-fluoro-5-((4-methyl-piperazin-1-yl)methyl)-1H-pyrazol-1-yl)-2-methylpropoxy)pyridin-4-yl)-4-fluoro-6-isopropylphenyl)acetic acid

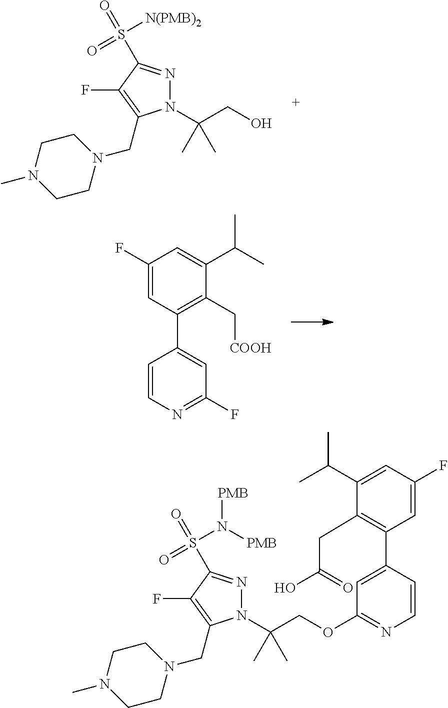

To a solution of 4-fluoro-1-(1-hydroxy-2-methylpropan-2-yl)-N,N-bis(4-methoxy-benzyl)-5-((4-methylpiperazin-1-yl)methyl)-1H-pyrazole-3-sulfonamide (Intermediate A30) (320 mg, 455 μmol, TFA salt) in THF (10 mL) was added NaH (46 mg, 1.14 mmol, 60% purity in mineral oil) at 0° C. The mixture was stirred at 0° C. for 0.5 h, then 2-(4-fluoro-2-(2-fluoropyridin-4-yl)-6-isopropylphenyl)acetic acid (Intermediate B14) (96 mg, 327 μmol, TFA salt) was added and the resulting mixture was stirred at 20° C. for 0.5 h. The mixture was heated to 50° C. for 12 h, then cooled to 20° C. and additional NaH (64 mg, 1.59 mmol, 60% purity in mineral oil) and 2-(4-fluoro-2-(2-fluoropyridin-4-yl)-6-isopropylphenyl)acetic acid (Intermediate B14) (184 mg, 455 μmol, TFA salt) were added. The reaction mixture was stirred at 50° C. for 24 h, then quenched with MeOH (5 mL) and adjusted to pH=7 with 1 M HCl. The mixture was concentrated in vacuum. The residue was purified by reverse phase flash chromatography (water (0.1% TFA)-MeCN) to give the title compound (200 mg, 45.1% yield, TFA salt) as a yellow solid.

LCMS: m/z 861.4 (M−TFA+H)⁺ (ES⁺).

¹H NMR (DMSO-d₆+D₂O): δ 8.11 (d, 1H), 7.15 (dd, 1H), 6.88 (d, 4H), 6.83 (dd, 1H), 6.69 (d, 4H), 6.59 (dd, 1H), 6.50 (s, 1H), 4.65 (s, 2H), 4.08 (s, 4H), 3.65-3.60 (m, 8H), 3.36-3.31 (m, 4H), 2.96-2.84 (m, 5H), 2.74 (s, 3H), 2.38-2.30 (m, 2H), 1.69 (s, 6H), 1.11 (d, 6H). One exchangeable proton and TFA proton not observed.

### Step B: 2-(4-fluoro-2-(2-(2-(4-fluoro-5-((4-methylpiperazin-1-yl)methyl)-3-sulfamoyl-1H-pyrazol-1-yl)-2-methylpropoxy)pyridin-4-yl)-6-isopropylphenyl)acetic acid

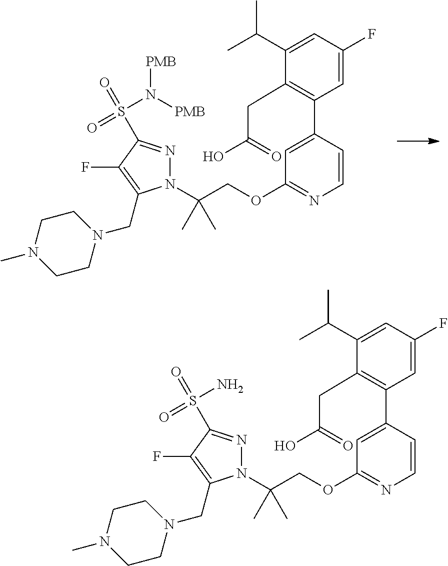

To a solution 2-(2-(2-(2-(3-(N,N-bis(4-methoxybenzyl)sulfamoyl)-4-fluoro-5-((4-methylpiperazin-1-yl)methyl)-1H-pyrazol-1-yl)-2-methylpropoxy)pyridin-4-yl)-4-fluoro-6-isopropylphenyl)acetic acid (200 mg, 205 μmol, TFA salt) in DCM (3 mL) was added TFA (4.62 g, 40.5 mmol). The mixture was stirred at 20° C. for 12 h, then treated with MeOH (3 mL), filtered and the filtrate was concentrated in vacuum. The residue was purified by reverse phase flash chromatography (water (0.1% TFA)-MeCN) to give the title compound (134 mg, 88.9% yield, TFA salt) as a yellow oil.

LCMS: m/z 621.2 (M−TFA+H)⁺ (ES⁺).

¹H NMR (DMSO-d₆): δ 12.5 (br s, 1H), 9.44 (br s, 1H), 8.20 (d, 1H), 7.71 (s, 2H), 7.24 (dd, 1H), 6.95-6.90 (m, 2H), 6.69 (s, 1H), 4.70 (s, 2H), 3.46 (s, 2H), 3.39 (d, 2H), 3.05-2.99 (m, 1H), 2.94-2.88 (m, 2H), 2.87-2.81 (m, 2H), 2.75 (s, 3H), 2.38-2.32 (m, 2H), 1.73 (s, 6H), 1.18 (d, 6H). One exchangeable proton not observed.

### Intermediate C54: 1-(1-((4-(2-(carboxymethyl)-5-fluoro-3-isopropylphenyl)pyridin-2-yl)oxy)-2-methylpropan-2-yl)-4-fluoro-3-sulfamoyl-1H-pyrazole-5-carboxylic acid

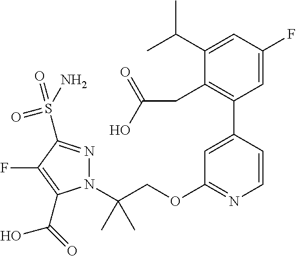 [SMILES: CC(C)c1cc(F)cc(-c2ccnc(OCC(C)(C)n3nc(S(N)(=O)=O)c(F)c3C(=O)O)c2)c1CC(=O)O]

### Step A: 3-(N,N-bis(4-methoxybenzyl)sulfamoyl)-1-(1-((4-(2-(carboxymethyl)-5-fluoro-3-isopropylphenyl)pyridin-2-yl)oxy)-2-methylpropan-2-yl)-4-fluoro-1H-pyrazole-5-carboxylic acid

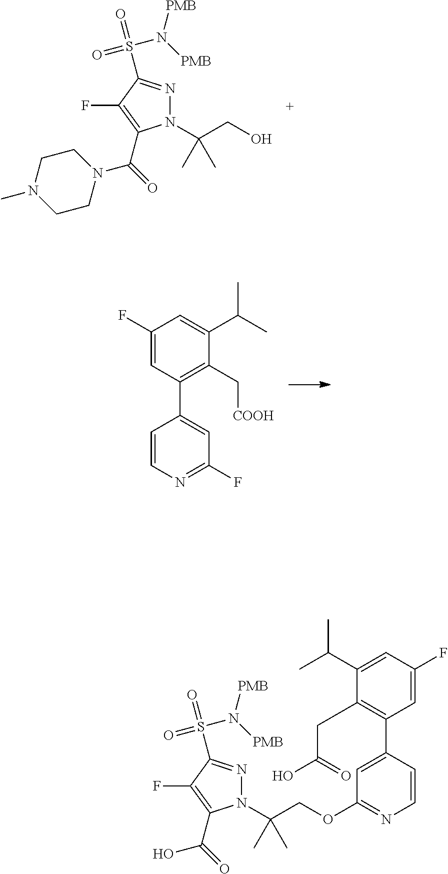

To a solution of 4-fluoro-1-(1-hydroxy-2-methylpropan-2-yl)-N,N-bis(4-methoxy-benzyl)-5-(4-methylpiperazine-1-carbonyl)-1H-pyrazole-3-sulfonamide (Intermediate A31) (250 mg, 348 μmol, TFA salt) in THF (15 mL) was added NaH (35 mg, 871 μmol, 60% purity in mineral oil) at 0° C. in portions under N₂. The mixture was stirred at 25° C. for 30 min, then 2-(4-fluoro-2-(2-fluoropyridin-4-yl)-6-isopropyl-phenyl)acetic acid (Intermediate B14) (112 mg, 275 μmol, TFA salt) was added and the resulting mixture was stirred at 25° C. for 1 h. The mixture was quenched with 1 M aq HCl (10 mL) at 0° C. and concentrated in vacuum. The residue was purified by reverse phase flash chromatography (water (0.1% TFA)-MeCN) to give the title compound (0.16 g, 57.9% yield) as a yellow solid.

LCMS: m/z 793.5 (M+H)⁺ (ES⁺).

¹H NMR (DMSO-d₆): δ 8.17 (d, 1H), 7.21 (dd, 1H), 7.01 (d, 4H), 6.88 (dd, 1H), 6.75 (d, 4H), 6.71 (dd, 1H), 6.58 (s, 1H), 4.80 (s, 2H), 4.24 (s, 4H), 3.69 (s, 6H), 3.40 (s, 2H), 3.01-2.98 (m, 1H), 1.73 (s, 6H), 1.17 (d, 6H). Two exchangeable protons not observed.

### Step B: 1-(1-((4-(2-(carboxymethyl)-5-fluoro-3-isopropylphenyl)pyridin-2-yl)oxy)-2-methylpropan-2-yl)-4-fluoro-3-sulfamoyl-1H-pyrazole-5-carboxylic acid

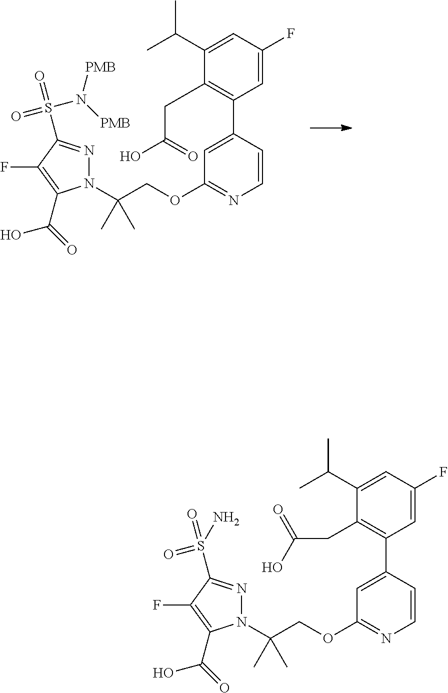

A solution of 3-(N,N-bis(4-methoxybenzyl)sulfamoyl)-1-(1-((4-(2-(carboxymethyl)-5-fluoro-3-isopropylphenyl)pyridin-2-yl)oxy)-2-methylpropan-2-yl)-4-fluoro-1H-pyrazole-5-carboxylic acid (160 mg, 202 μmol) in DCM (3 mL) and TFA (3 mL) was stirred at 25° C. for 1 h. The mixture was concentrated in vacuum. The residue was purified by reverse phase flash chromatography (water (0.1% TFA)-MeCN) to give the title compound (0.13 g, 96.6% yield, TFA salt) as a yellow solid.

LCMS: m/z 553.3 (M-TFA+H)⁺ (ES⁺).

¹H NMR (CD₃OD): δ 8.11 (d, 1H), 7.13 (dd, 1H), 6.94 (dd, 1H), 6.81 (dd, 1H), 6.73 (s, 1H), 4.91 (s, 2H), 3.54 (s, 2H), 3.14-3.11 (m, 1H), 1.78 (s, 6H), 1.24 (d, 6H). Four exchangeable protons and TFA proton not observed.

### Intermediate C55: 2-(4-fluoro-2-(2-(2-(4-fluoro-5-(morpholinomethyl)-3-sulfamoyl-1H-pyrazol-1-yl)-2-methylpropoxy)pyridin-4-yl)-6-isopropylphenyl)acetic acid

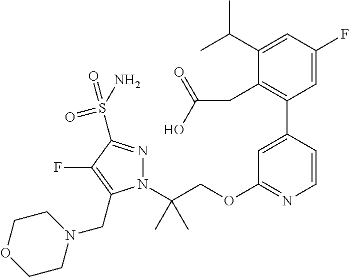 [SMILES: CC(C)c1cc(F)cc(-c2ccnc(OCC(C)(C)n3nc(S(N)(=O)=O)c(F)c3CN3CCOCC3)c2)c1CC(=O)O]

### Step A: 2-(2-(2-(2-(3-(N,N-bis(4-methoxybenzyl)sulfamoyl)-4-fluoro-5-(morpholino-methyl)-1H-pyrazol-1-yl)-2-methylpropoxy)pyridin-4-yl)-4-fluoro-6-isopropylphenyl)-acetic acid

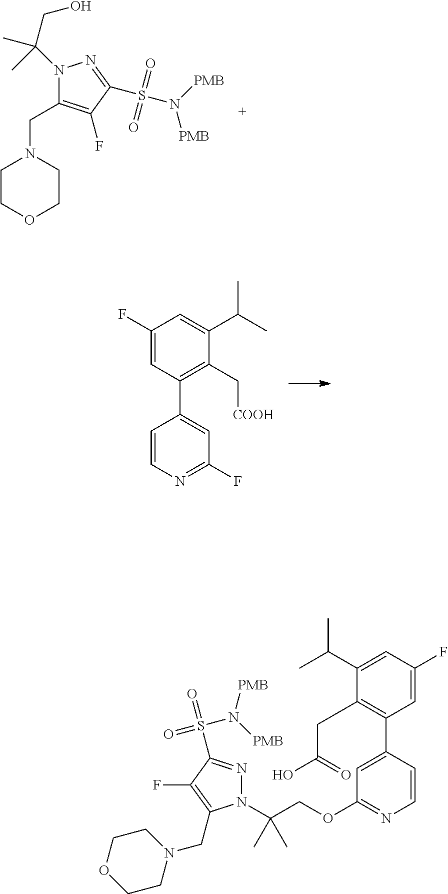

To a solution of 4-fluoro-1-(1-hydroxy-2-methylpropan-2-yl)-N,N-bis(4-methoxy-benzyl)-5-(morpholinomethyl)-1H-pyrazole-3-sulfonamide (Intermediate A32) (0.3 g, 520 μmol) in THF (3 mL) was added NaH (73 mg, 1.82 mmol, 60% purity in mineral oil) at 0° C. under N₂. The mixture was stirred at 0° C. for 0.5 h, then a solution of 2-(4-fluoro-2-(2-fluoropyridin-4-yl)-6-isopropylphenyl)acetic acid (Intermediate B14) (232 mg, 572 μmol, TFA salt) in THF (1 mL) was added to the mixture at 0° C. The reaction mixture was stirred at 20° C. for 12 h under N₂, then quenched with 0.5 M aq HCl and extracted with EtOAc (50 mL×2). The combined organic phases were dried over anhydrous Na₂SO₄, filtered and concentrated in vacuum. The residue was purified by reverse phase flash chromatography (water (0.1% NH₃.H₂O)-MeCN) to give the title compound (0.15 g, 34.0% yield) as a yellow solid.

LCMS: m/z 848.2 (M+H)⁺ (ES⁺).

¹H NMR (CDCl₃): δ 8.23 (d, 1H), 7.12-7.09 (m, 5H), 6.96 (d, 1H), 6.88 (s, 1H), 6.80-6.76 (m, 5H), 4.71 (s, 2H), 4.37 (s, 4H), 4.31 (s, 2H), 3.99-3.96 (m, 4H), 3.77 (s, 6H), 3.52 (s, 2H), 3.39-3.20 (m, 3H), 3.11-3.05 (m, 2H), 1.84 (s, 6H), 1.26 (d, 6H). One exchangeable proton not observed.

### Step B: 2-(4-fluoro-2-(2-(2-(4-fluoro-5-(morpholinomethyl)-3-sulfamoyl-1H-pyrazol-1-yl)-2-methylpropoxy)pyridin-4-yl)-6-isopropylphenyl)acetic acid

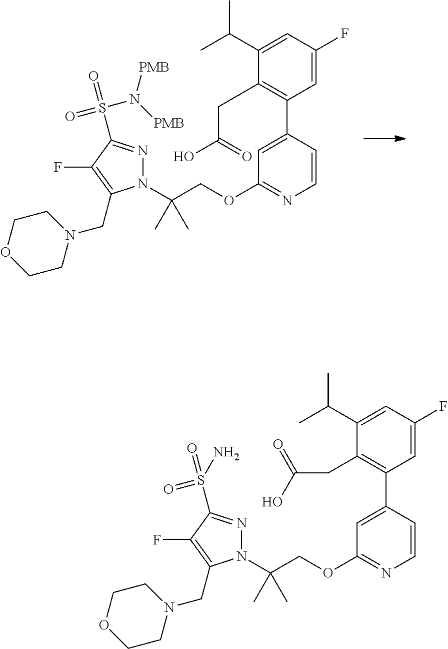

To a solution of 2-(2-(2-(2-(3-(N,N-bis(4-methoxybenzyl)sulfamoyl)-4-fluoro-5-(morpholinomethyl)-1H-pyrazol-1-yl)-2-methylpropoxy)pyridin-4-yl)-4-fluoro-6-isopropylphenyl)acetic acid (150 mg, 177 μmol) in DCM (3 mL) was added TFA (6.00 mL, 81.0 mmol). The mixture was stirred at 20° C. for 12 h, then concentrated in vacuum. The residue was purified by reverse phase flash chromatography (water (0.1% TFA)-MeCN) to give the title compound (0.1 g, 78.3% yield) as a yellow solid.

LCMS: m/z 608.3 (M+H)⁺ (ES⁺).

¹H NMR (DMSO-d₆): δ 8.20 (d, 1H), 7.71 (s, 2H), 7.23 (dd, 1H), 6.93-6.88 (m, 2H), 6.67 (s, 1H), 4.73 (s, 2H), 3.55 (s, 3H), 3.51-3.41 (m, 7H), 3.04-2.99 (m, 1H), 2.35-2.33 (m, 2H), 1.73 (s, 6H), 1.18 (d, 6H). One exchangeable proton not observed.

### Intermediate C56: 2-(4-fluoro-2-isopropyl-6-(2-((2-methyl-2-(4-sulfamoyl-1H-pyrazol-1-yl)propyl)amino)pyridin-4-yl)phenyl)acetic acid

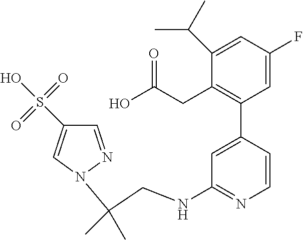 [SMILES: CC(C)c1cc(F)cc(-c2ccnc(NCC(C)(C)n3cc(S(=O)(=O)O)cn3)c2)c1CC(=O)O]

### Step A: 2-(2-(2-((2-(4-(N,N-bis(4-methoxybenzyl)sulfamoyl)-1H-pyrazol-1-yl)-2-methylpropyl)amino)pyridin-4-yl)-4-fluoro-6-isopropylphenyl)acetic acid

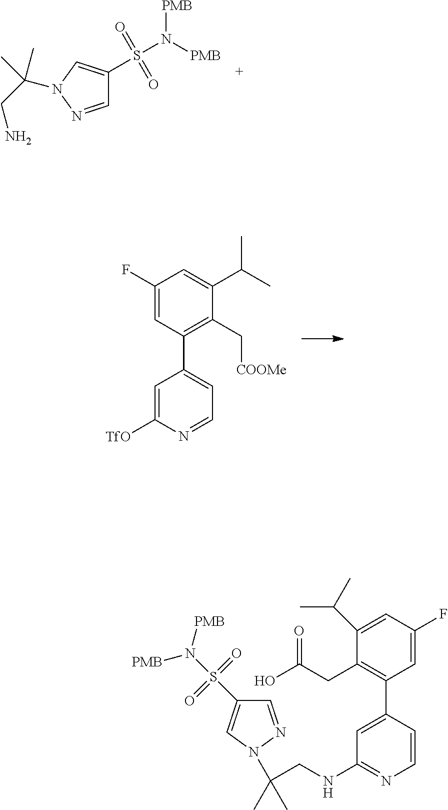

To a solution of 1-(1-amino-2-methylpropan-2-yl)-N,N-bis(4-methoxybenzyl)-1H-pyrazole-4-sulfonamide (Intermediate A33) (700 mg, 1.41 mmol, HCl salt) in 2-methylbutan-2-ol (20 mL) was added methyl 2-(4-fluoro-2-isopropyl-6-(2-(((trifluoro-methyl)sulfonyl)oxy)pyridin-4-yl)phenyl)acetate (Intermediate B17) (616 mg, 1.41 mmol), LiHMDS (1 M in THF, 2.83 mL) and [2-(2-aminophenyl)phenyl]-methyl-sulfonyloxy-palladium; di-tert-butyl-[2-(2,4,6-triisopropylphenyl)phenyl]phosphane (112 mg, 141 μmol) at 25° C. under N₂. The mixture was stirred at 90° C. for 12 h, then quenched with 1 M aq HCl (3 mL) and concentrated in vacuum. The residue was purified by reverse phase flash chromatography (water (0.1% TFA)-MeCN) to give the title compound (0.12 g, 11.6% yield) as a yellow solid.

LCMS: m/z 730.3 (M+H)⁺ (ES⁺).

¹H NMR (CD₃OD): δ 7.90 (s, 1H), 7.82 (d, 1H), 7.64 (s, 1H), 7.23 (dd, 1H), 7.11 (d, 4H), 6.89-6.78 (m, 7H), 4.21 (s, 4H), 3.85 (s, 2H), 3.75 (s, 6H), 3.57 (s, 2H), 3.17-3.14 (m, 1H), 1.66 (s, 6H), 1.26 (d, 6H). Two exchangeable protons not observed.

### Step B: 2-(4-fluoro-2-isopropyl-6-(2-((2-methyl-2-(4-sulfamoyl-1H-pyrazol-1-yl)-propyl)amino)pyridin-4-yl)phenyl)acetic acid

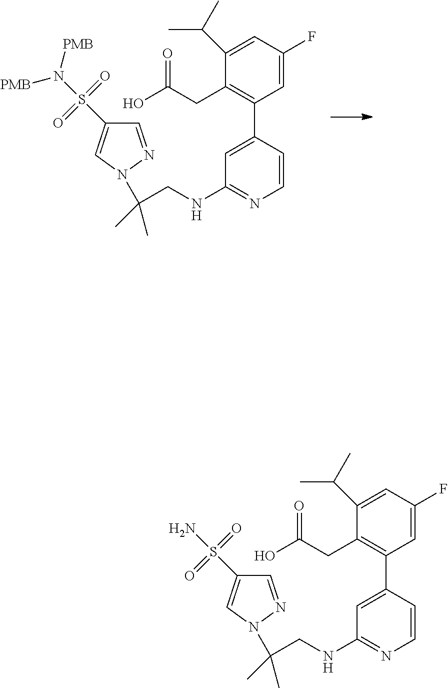

A solution of 2-(2-(2-((2-(4-(N,N-bis(4-methoxybenzyl)sulfamoyl)-1H-pyrazol-1-yl)-2-methylpropyl)amino)pyridin-4-yl)-4-fluoro-6-isopropylphenyl)acetic acid (100 mg, 137 μmol) in DCM (0.5 mL) and TFA (0.5 mL) was stirred at 25° C. for 1 h. The solution was concentrated in vacuum. The residue was purified by reverse phase flash chromatography (water (0.1% TFA)-MeCN) to give the title compound (82 mg, 99.2% yield, TFA salt) as a yellow solid.

LCMS: m/z 490.3 (M-TFA+H)⁺ (ES⁺).

¹H NMR (CDCl₃): δ 7.94-7.90 (m, 2H), 7.72 (s, 1H), 6.96 (dd, 1H), 6.66 (dd, 1H), 6.51 (dd, 1H), 6.05 (s, 1H), 4.64-4.61 (m, 1H), 3.72 (d, 2H), 3.40 (s, 2H), 3.22-3.01 (m, 1H), 1.64 (s, 6H), 1.25 (d, 6H). Three exchangeable protons not observed.

### Intermediate D1: 2-(2-isopropyl-6-(2-(2-(methyl(1-sulfamoylpiperidin-3-yl)amino)-ethoxy)pyridin-4-yl)phenyl)acetic acid

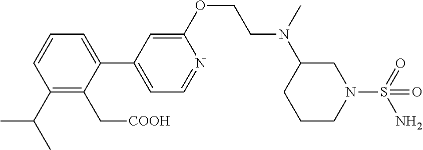 [SMILES: CC(C)c1cccc(-c2ccnc(OCCN(C)C3CCCN(S(N)(=O)=O)C3)c2)c1CC(=O)O]

### Step A: 2-(2-(2-(2,2-diethoxyethoxy)pyridin-4-yl)-6-isopropylphenyl)acetic acid

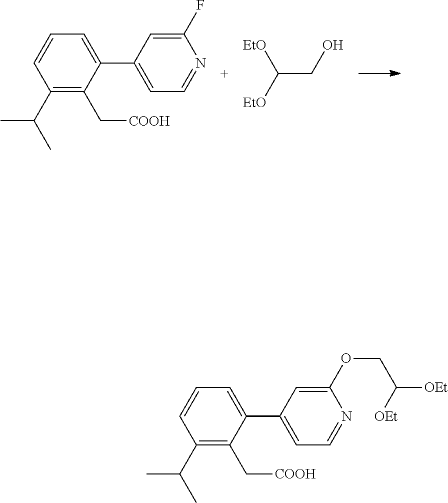 [SMILES: CC(C)c1cccc(-c2ccnc(F)c2)c1CC(=O)O ; CCOC(CO)OCC ; CCOC(COc1cc(-c2cccc(C(C)C)c2CC(=O)O)ccn1)OCC]

To a solution of 2,2-diethoxyethanol (1.47 g, 11.0 mmol) in DMF (40 mL) was added NaH (439 mg, 11.0 mmol, 60% purity in mineral oil) in portions at 0° C. under N₂. Then to the above solution was added 2-(2-(2-fluoropyridin-4-yl)-6-isopropylphenyl)acetic acid (Intermediate B1) (1.00 g, 3.66 mmol) in portions and the resulting mixture was stirred at 40° C. for 3 h. The mixture was quenched with H₂O (60 mL) and then extracted with EtOAc (2×40 mL). The combined organic phases were washed with brine (2×50 mL), dried over anhydrous Na₂SO₄, filtered and concentrated in vacuum to give the title compound (1.3 g, crude) as a colourless oil.

LCMS: m/z 388.1 (M+H)⁺ (ES⁺).

¹H NMR (CDCl₃): δ 8.14 (d, 1H), 7.39-7.32 (m, 2H), 7.05 (d, 1H), 6.86 (d, 1H), 6.75 (s, 1H), 4.89 (t, 1H), 4.40 (d, 2H), 3.78-3.72 (m, 4H), 3.67 (s, 2H), 3.11-3.04 (m, 1H), 1.26-1.22 (m, 12H). One exchangeable proton not observed.

### Step B: 2-(2-isopropyl-6-(2-(2-oxoethoxy)pyridin-4-yl)phenyl)acetic acid

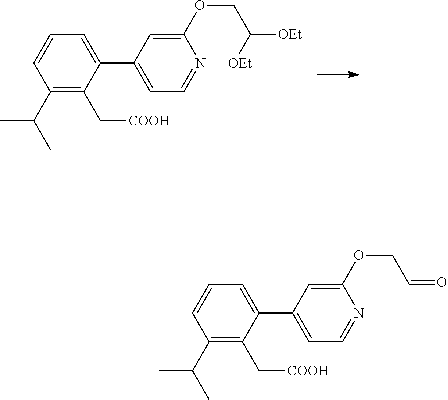 [SMILES: CCOC(COc1cc(-c2cccc(C(C)C)c2CC(=O)O)ccn1)OCC ; CC(C)c1cccc(-c2ccnc(OCC=O)c2)c1CC(=O)O]

To a solution of 2-(2-(2-(2,2-diethoxyethoxy)pyridin-4-yl)-6-isopropylphenyl)acetic acid (1.10 g, 2.84 mmol) in dioxane (21 mL) and H₂O (30 mL) was added conc H₂SO₄ (1.11 g, 11.4 mmol) and the mixture was stirred at 80° C. for 4 h. The reaction mixture was adjusted to pH 7 with sat aq Na₂CO₃ solution. The reaction mixture was concentrated in vacuum to remove most of the dioxane. The aqueous layer was purified directly by reverse phase flash chromatography (0.1% TFA in H₂O-MeCN) to give the title compound (1.12 g, 84.6% yield over two steps, TFA salt) as a light yellow solid.

LCMS: m/z 314.1 (M+H)⁺ (ES⁺).

¹H NMR (CD₃OD): δ 8.58 (d, 1H), 7.60-7.54 (m, 2H), 7.46-7.33 (m, 1H), 7.17 (d, 1H), 6.95-6.71 (m, 1H), 4.93-4.90 (m, 2H), 3.67 (s, 2H), 3.31-3.11 (m, 1H), 1.28-1.25 (m, 6H). Two protons not observed. TFA proton not observed.

### Step C: 2-(2-(2-(2-((1-(tert-butoxycarbonyl)piperidin-3-yl)(methyl)amino)ethoxy)-pyridin-4-yl)-6-isopropylphenyl)acetic acid

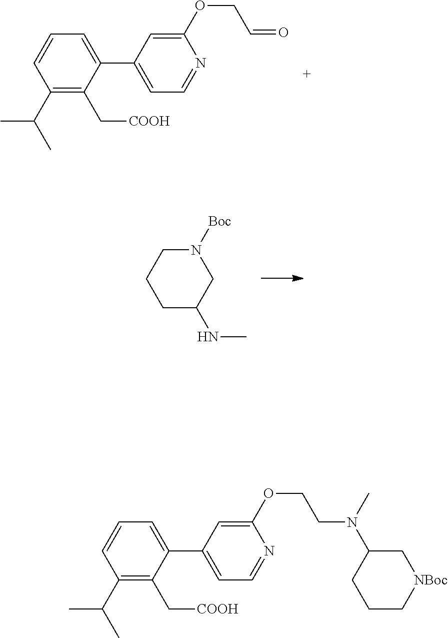 [SMILES: CC(C)c1cccc(-c2ccnc(OCC=O)c2)c1CC(=O)O ; CNC1CCCN(C(=O)OC(C)(C)C)C1 ; CC(C)c1cccc(-c2ccnc(OCCN(C)C3CCCN(C(=O)OC(C)(C)C)C3)c2)c1CC(=O)O]

To a solution of 2-(2-isopropyl-6-(2-(2-oxoethoxy)pyridin-4-yl)phenyl)acetic acid (330 mg, 772 μmol, TFA salt) in MeOH (2 mL) and AcOH (0.01 mL) was added tert-butyl 3-(methylamino)piperidine-1-carboxylate (248 mg, 1.16 mmol) and the mixture was stirred at 25° C. for 30 min. To above mixture was added NaBH₃CN (146 mg, 2.32 mmol) and the resulting mixture was stirred at 25° C. for 1 h. The mixture was concentrated in vacuum. The residue was purified by reverse phase flash chromatography (0.1% TFA in H₂O-MeCN) to give the title compound (0.3 g, 62.1%, di-TFA salt) as a white solid.

LCMS: m/z 512.3 (M+H)⁺ (ES⁺).

¹H NMR (CDCl₃): δ 8.24 (d, 1H), 7.43-7.32 (m, 2H), 7.05-6.95 (m, 3H), 4.80-4.75 (m, 2H), 4.51-4.45 (m, 1H), 4.02-3.95 (m, 1H), 3.68-3.57 (m, 4H), 3.34-3.30 (m, 1H), 3.14-3.10 (m, 1H), 3.02-2.76 (m, 5H), 2.31-2.28 (m, 1H), 1.88-1.85 (m, 1H), 1.73-1.61 (m, 1H), 1.59-1.54 (m, 1H), 1.43 (s, 9H), 1.26 (d, 6H). One exchangeable proton not observed. TFA protons not observed.

### Step D: methyl 2-(2-isopropyl-6-(2-(2-(methyl(piperidin-3-yl)amino)ethoxy)pyridin-4-yl)phenyl)acetate

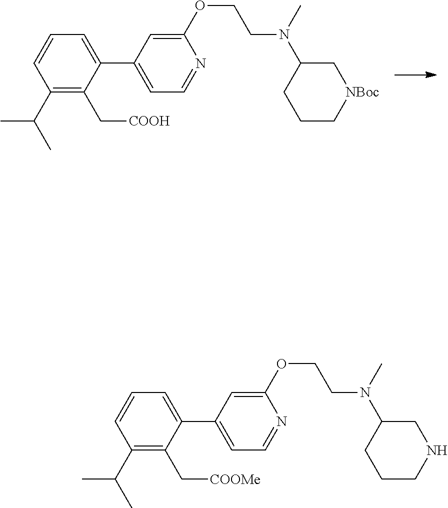 [SMILES: CC(C)c1cccc(-c2ccnc(OCCN(C)C3CCCN(C(=O)OC(C)(C)C)C3)c2)c1CC(=O)O ; COC(=O)Cc1c(-c2ccnc(OCCN(C)C3CCCNC3)c2)cccc1C(C)C]

To a solution of 2-(2-(2-(2-((1-(tert-butoxycarbonyl)piperidin-3-yl)(methyl)amino)-ethoxy)pyridin-4-yl)-6-isopropylphenyl)acetic acid (0.3 g, 479 μmol, di-TFA salt) in MeOH (20 mL) was added conc H₂SO₄ (47 mg, 479 μmol). Then the mixture was stirred at 60° C. for 12 h. The mixture was concentrated in vacuum. The residue was purified by reverse phase flash chromatography (0.1% TFA in H₂O-MeCN) to give the title compound (0.3 g, 81.5%, tri-TFA salt) as a yellow oil.

LCMS: m/z 426.3 (M+H)⁺ (ES⁺).

¹H NMR (CDCl₃): δ 8.14 (d, 1H), 7.39-7.33 (m, 2H), 7.02 (d, 1H), 6.96 (d, 1H), 6.79 (s, 1H), 4.74 (s, 2H), 4.11-4.02 (m, 1H), 3.66-3.60 (m, 8H), 3.51-3.25 (m, 2H), 3.06-3.01 (m, 1H), 3.01-2.91 (m, 4H), 2.30-2.28 (m, 1H), 2.15-1.77 (m, 3H), 1.24 (d, 6H). One exchangeable proton not observed. TFA protons not observed.

### Step E: methyl 2-(2-isopropyl-6-(2-(2-(methyl(1-sulfamoylpiperidin-3-yl)amino)-ethoxy)pyridin-4-yl)phenyl)acetate

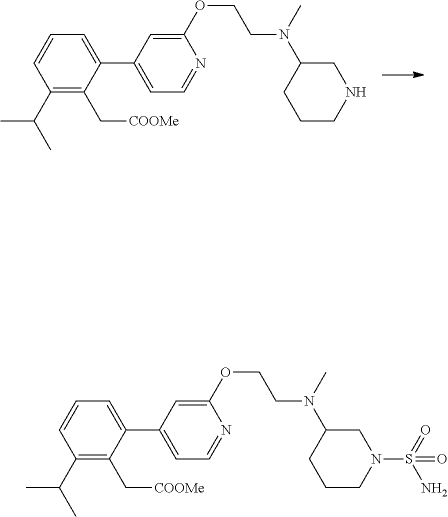 [SMILES: COC(=O)Cc1c(-c2ccnc(OCCN(C)C3CCCNC3)c2)cccc1C(C)C ; COC(=O)Cc1c(-c2ccnc(OCCN(C)C3CCCN(S(N)(=O)=O)C3)c2)cccc1C(C)C]

To a solution of methyl 2-(2-isopropyl-6-(2-(2-(methyl(piperidin-3-yl)amino)ethoxy)-pyridin-4-yl)phenyl)acetate (0.25 g. 326 μmol, tri-TFA salt) in dioxane (5 mL) was added DIPEA (168 mg, 1.30 mmol) and sulfuric diamide (188 mg, 1.95 mmol). Then the solution was stirred at 110° C. for 2 h under microwave irradiation. The solution was concentrated in vacuum. The residue was purified by reverse phase flash chromatography (0.1% TFA in H₂O-MeCN) to give the title compound (90 mg, 37.7%, di-TFA salt) as a white solid.

LCMS: m/z 505.4 (M+H)⁺ (ES⁺).

¹H NMR (CD₃OD): δ 8.21 (d, 1H), 7.43-7.35 (m, 2H), 7.04 (d, 1H), 6.98 (dd, 1H), 6.84 (s, 1H), 4.79-4.74 (m, 2H), 3.69-3.54 (m, 9H), 3.30-3.27 (m, 2H), 3.10-3.05 (m, 5H), 2.07-1.78 (m, 4H), 1.24 (d, 6H). Two exchangeable protons not observed. TFA protons not observed.

### Step F: 2-(2-isopropyl-6-(2-(2-(methyl(1-sulfamoylpiperidin-3-yl)amino)ethoxy)-pyridin-4-yl)phenyl)acetic acid

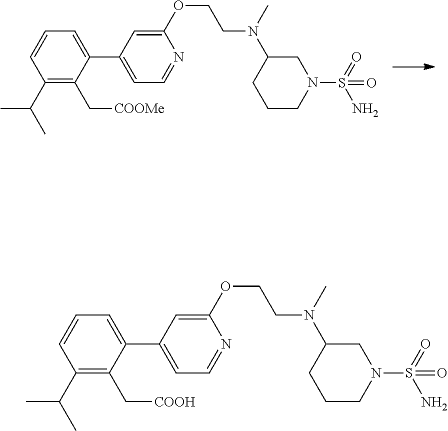 [SMILES: COC(=O)Cc1c(-c2ccnc(OCCN(C)C3CCCN(S(N)(=O)=O)C3)c2)cccc1C(C)C ; CC(C)c1cccc(-c2ccnc(OCCN(C)C3CCCN(S(N)(=O)=O)C3)c2)c1CC(=O)O]

To a solution of methyl 2-(2-isopropyl-6-(2-(2-(methyl(1-sulfamoylpiperidin-3-yl)-amino)ethoxy)pyridin-4-yl)phenyl)acetate (90 mg, 123 μmol, di-TFA salt) in MeOH (3 mL) and H₂O (2 mL) was added LiOH.H₂O (21 mg, 491 μmol) and the solution was stirred at 40° C. for 3 h. The solution was adjusted to pH=7 with 1 M HCl and the mixture was concentrated in vacuum to remove most of the methanol. The aqueous layer was purified by reverse phase flash chromatography (0.1% TFA in H₂O-MeCN) to give the title compound (85 mg, 96.3%, di-TFA salt) as a colourless oil.

LCMS: m/z 491.2 (M+H)⁺ (ES⁺).

¹H NMR (CDCl₃): δ 8.19 (d, 1H), 7.53-7.27 (m, 2H), 7.04 (d, 1H), 6.95 (dd, 1H), 6.83 (s, 1H), 4.73 (s, 2H), 3.69-3.58 (m, 7H), 3.27-3.22 (m, 1H), 3.19-2.81 (m, 5H), 1.90-1.65 (m, 4H), 1.26-1.22 (m, 6H). Three exchangeable protons not observed. TFA protons not observed.

### Preparation of Examples

### Example 1: 16,16-dimethyl-6-(propan-2-yl)-18-oxa-11λ⁶-thia-10,15,20,24-tetraaza-tetracyclo[17-3-1-1^{12,15}.0^{2,7}]tetracosa-1(22),2(7),3,5,12(24),13,19(23),20-octaene-9,11,11-trione

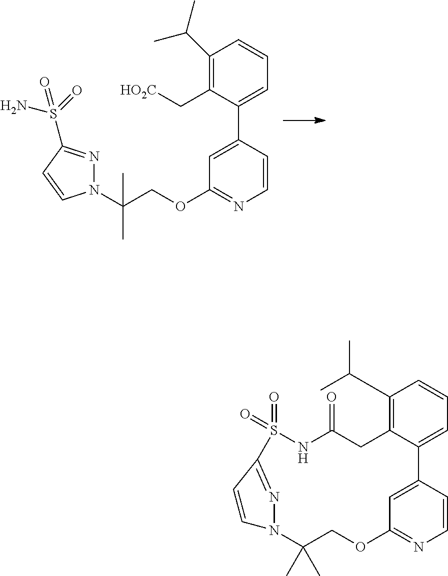 [SMILES: CC(C)c1cccc(-c2ccnc(OCC(C)(C)n3ccc(S(N)(=O)=O)n3)c2)c1CC(=O)O ; CC(C)c1cccc2c1CC(=O)NS(=O)(=O)c1ccn(n1)C(C)(C)COc1cc-2ccn1]

DMAP (70.8 mg, 0.580 mmol) and EDC (111 mg, 0.580 mmol) were added to a solution of 2-(2-isopropyl-6-(2-(2-methyl-2-(3-sulfamoyl-1H-pyrazol-1-yl)propoxy)pyridin-4-yl)phenyl)acetic acid (Intermediate C2) (137 mg, 0.290 mmol) in DMF (2 mL) and the reaction was stirred at RT for 18 h. The crude product was purified by acidic prep-HPLC (50-80% MeCN in water) to afford the title compound (10.78 mg, 8%) as a flocculent white solid.

LCMS m/z 455.2 (M+H)⁺ (ES⁺); 453.2 (M−H)⁻ (ES⁻).

¹H NMR (DMSO-d₆) δ 11.93 (s, 1H), 8.22 (d, J=5.1 Hz, 1H), 8.03 (s, 1H), 7.38-7.32 (m, 1H), 7.29 (t, J=7.6 Hz, 1H), 6.94-6.90 (m, 1H), 6.88 (d, J=5.1 Hz, 1H), 6.71 (s, 1H), 5.73 (s, 1H), 4.50 (s, 2H), 3.41-3.35 (m, 2H), 2.97-2.92 (m, 1H), 1.66 (s, 6H), 1.17 (d, J=6.7 Hz, 6H).

The following examples ‘Ex’ were synthesised following the general procedure for Example 1, from the intermediate compounds indicated in the ‘From’ column:

| Ex | Structure | From | ¹H NMR | LCMS |
|---|---|---|---|---|
| 2 | 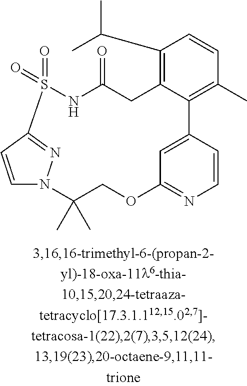 [SMILES: Cc1ccc(C(C)C)c2c1-c1ccnc(c1)OCC(C)(C)n1ccc(n1)S(=O)(=O)NC(=O)C2] | C3 | ¹H NMR (DMSO-d₆) (major conformer) δ 11.86 (br s, 1H), 8.26 (d, J = 5.2 Hz, 1H), 8.05- 8.00 (m, 1H), 7.23 (d, J = 8.0 Hz, 1H), 7.16 (d, J = 8.1 Hz, 1H), 6.79 (d, J = 5.1 Hz, 1H), 6.70 (s, 1H), 5.63 (s, 1H), 4.72 (d, J = 10.6 Hz, 1H), 4.27 (d, J = 10.5 Hz, 1H), 3.57-3.53 (m, 1H), 2.97-2.93 (m, 1H), 2.87 (sept, J = 6.8 Hz, 1H), 1,86 (s, 3H), 1.82 (s, 3H), 1.52 (s, 3H), 1.16 (d, J = 6.8 Hz, 3H), 1.12 (d, J = 6.8 Hz, 3H). | m/z 469.2 (M + H)⁺ (ES⁺) 467.1 (M − H)⁻ (ES⁻) |
| 3 | 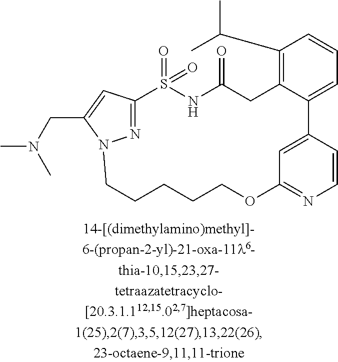 [SMILES: CC(C)c1cccc2c1CC(=O)NS(=O)(=O)c1cc(CN(C)C)n(n1)CCCCCOc1cc-2ccn1] | C1 | ¹H NMR (DMSO-d₆) δ 8.19 (d, J = 5.2 Hz, 1H), 8.14 (s, 1H), 7.41 (dd, J = 7.9 Hz, 1.4 Hz, 1H), 7.34 (t, J = 7.6 Hz, 1H), 7.09 (br s, 1H), 7.02 (dd, J = 7.5, 1.4 Hz, 1H), 6.85 (dd, J = 5.2, 1.5 Hz, 1H), 6.30- 6.27 (m, 1H), 4.45-4.22 (m, 6H), 3.59-347 (m, 2H), 2.90 (sept, J = 6.4 Hz, 1H), 7.76-2.63 (m, 6H), 1.90-1.82 (m, 2H), 1.73-1.65 (m, 2H), 1.34- 1.24 (m, 2H), 1.20 (d, J = 6.8 Hz, 6H). | m/z 526.3 (M + H)⁺ (ES⁺) 524.2 (M − H)⁻ (ES⁻) |
| 4 | 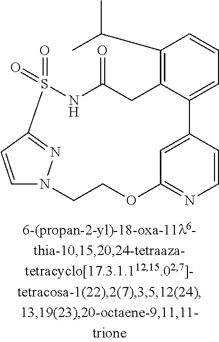 [SMILES: CC(C)c1cccc2c1CC(=O)NS(=O)(=O)c1ccn(n1)CCOc1cc-2ccn1] | C4 | ¹H NMR (DMSO-d₆) δ 12.03 (br s, 1H), 8.23 (d, J = 5.2 Hz, 1H), 8.03 (br s, 1H), 7.39-7.33 (m, 1H), 7.33-7.27 (m, 1H), 6.96 (d, J = 7.3 Hz, 1H), 6.90 (d, J = 5.1 Hz, 1H), 6.74 (s, 1H), 6.01 (br s, 1H), 4.75 (br s, 2H),4.55 (br s, 2H), 3.45-3.37 (m, 2H), 2.95-2.87 (m, 1H), 1.18 (d, J = 6.7 Hz, 6H). | m/z 427.1 (M + H)⁺ (ES⁺) 425.2 (M − H)⁻ (ES⁻) |
| 5 | 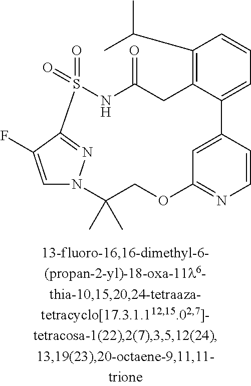 [SMILES: CC(C)c1cccc2c1CC(=O)NS(=O)(=O)c1nn(cc1F)C(C)(C)COc1cc-2ccn1] | C5 | ¹H NMR (DMSO-d₆) δ 12.14 (br s, 1H), 8.21 (d, J = 5.2 Hz, 2H), 7.35 (d, J = 7.8 Hz, 1H), 7.29 (t, J = 7.6 Hz, 1H), 6.98-6.90 (m, 1H), 6.88 (d, J = 5.1 Hz, 1H), 5.88 (br s, 1H), 4.49 (s, 2H), 3.37 (s, 2H), 2.96 (sept, J = 6.8 Hz, 1H), 1.61 (s, 6H), 1.17 (d, J = 6.8 Hz, 6H). | m/z 473.4 (M + H)⁺ (ES⁺) 471.3 (M − H)⁻ (ES⁻) |
| 7 | 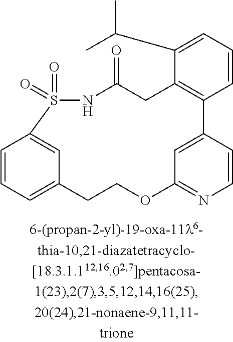 [SMILES: CC(C)c1cccc2c1CC(=O)NS(=O)(=O)c1cccc(c1)CCOc1cc-2ccn1] | C7 | ¹H NMR (DMSO-d₆) δ 12.40-11.46 (br s, 1H), 8.19 (d, J = 5.2 Hz, 1H), 7.63 (dd, J = 7.6, 1.8 Hz, 2H), 7.49 (t, J = 7.7 Hz, 1H), 7.34 (d,J = 7.8 Hz, 1H), 7.27 (t, J = 7.6 Hz, 1H), 7.13 (s, 1H), 6.93 (dd, J = 7.3,1.4 Hz, 1H), 6.86 (d, J = 5.2 Hz, 1H), 6.09 (s, 1H), 4.69 (br s, 2H), 3.49 (br s, 2H), 3.08 (t, J = 5.8 Hz, 2H), 2.96 (sept, J = 6.7 Hz, 1H), 1.18 (d, J = 6.8 Hz, 6H). | m/z 437.5 (M + H)⁺ (ES⁺) 435.3 (M − H)⁻ (ES⁻) |
| 8 | 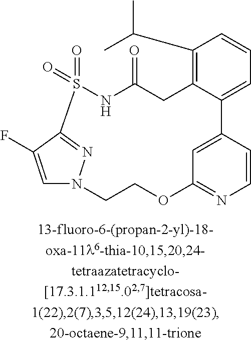 [SMILES: CC(C)c1cccc2c1CC(=O)NS(=O)(=O)c1nn(cc1F)CCOc1cc-2ccn1] | C8 | ¹H NMR (DMSO-d₆) δ 12.29 (s, 1H), 8.23 (d, J = 5.2 Hz, 2H), 7.37 (d, J = 7.8 Hz, 1H), 7.31 (t, J = 7.7 Hz, 1H), 6.98 (d, J = 7.1Hz, 1H), 6.92 (d, J = 5.1 Hz, 1H), 6.13 (s, 1H), 4-74 (br s, 1H), 4.46 (br s, 2H), 3.50-3-37 (m, 4H), 1.18 (d, J = 6.7 Hz, 6H). | m/z 445.4 (M + H)⁺ (ES⁺) 443.3 (M − H)⁻ (ES⁻) |
| 9 | 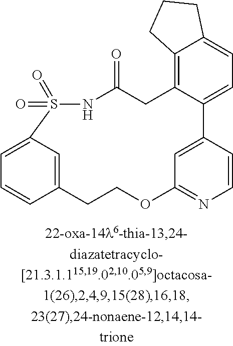 [SMILES: O=C1Cc2c(ccc3c2CCC3)-c2ccnc(c2)OCCc2cccc(c2)S(=O)(=O)N1] | C9 | ¹H NMR (DMSO-d₆) δ 11.93 (br s, 1H), 8.13 (d, J = 5.2 Hz, 1H), 7.68 (d, J = 7.7 Hz, 1H), 7.66-7.63 (m, 1H), 7.51 (t, J = 7.7 Hz, 1H), 7.19-7.14 (m, 2H), 6.89 (d, J = 7.6 Hz, 1H), 6.80 (d, J = 5.2 Hz, 1H), 6.03 (br s, 1H), 4.71 (br s, 2H), 3.51 (br s, 2H), 3.11 (t, J = 5.8 Hz, 2H), 2.91 (t, J = 7.5 Hz, 2H), 2.79 (t, J = 7.4 Hz, 2H), 2.03 (p, J = 7.5 Hz, 2H). | m/z 435.5 (M + H)⁺ (ES⁺) 433.3 (M − H)⁻ (ES⁻) |
| 10 | 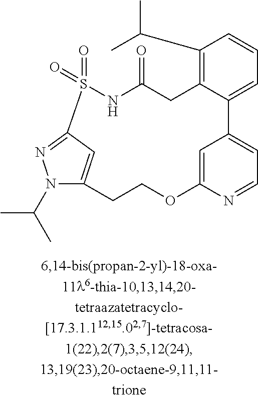 [SMILES: CC(C)c1cccc2c1CC(=O)NS(=O)(=O)c1cc(n(C(C)C)n1)CCOc1cc-2ccn1] | C10 | ¹H NMR (DMSO-d₆) δ 11.87 (s, 1H), 7.89 (s, 1H), 7.38-7.23 (m, 2H), 6.95 (dd, J = 7.3, 1.5 Hz, 1H), 6.83-6.68 (m, 1H), 6.30 (s, 1H), 6.10 (s, 1H), 5.03-4.58 (m, 3H), 3.61 (s, 2H), 3.13 (t, J = 5.6 Hz, 2H), 3.02 (dt, J = 12.5, 6.7 Hz, 1H), 1.43 (d, J = 6.5 Hz, 6H), 1.17 (d, J = 6.7 Hz, 6H). | m/z 469.5 (M + H)⁺ (ES⁺) |
| 11 | 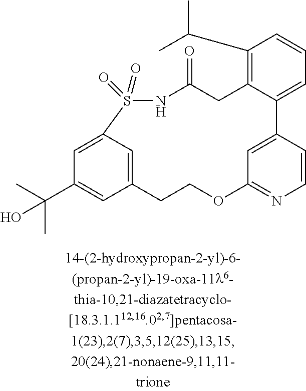 [SMILES: CC(C)c1cccc2c1CC(=O)NS(=O)(=O)c1cc(cc(C(C)(C)O)c1)CCOc1cc-2ccn1] | C11 | ¹H NMR (DMSO-d₆) δ 8.23-8.16 (m, 1H), 7.79 (s, 1H), 7.73-7-68 (m, 1H), 7.36 (dd, J = 7.9, 1.4 Hz, 1H), 7.30 (t, J = 7.6 Hz, 1H), 6.99 (s, 1H), 6.96 (dd, J = 7.5, 1.4 Hz, 1H), 6.86 (d, J = 4-3 Hz, 1H), 6.11 (s, 1H), 5.27 (s, 1H), 4.69 (s, 2H), 3.52 (s, 2H), 3.08 (t, J = 5.5 Hz, 2H), 3.00-2.90 (m, 1H), 1.45 (s, 6H), 1.19 (d, J = 6.8 Hz, 6H). One exchangeable proton not observed. | m/z 495.1 (M + H)⁺ (ES⁺) |
| 12 | 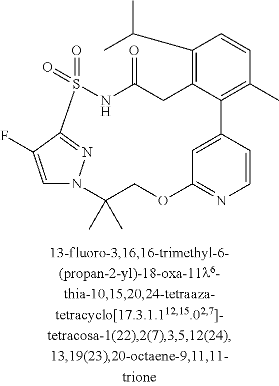 [SMILES: Cc1ccc(C(C)C)c2c1-c1ccnc(c1)OCC(C)(C)n1cc(F)c(n1)S(=O)(=O)NC(=O)C2] | C12 | ¹H NMR (DMSO-d₆) δ 12.07 (s, 1H), 8.35-8.18 (m, 2H), 7.24 (d, J = 8.0 Hz, 1H), 7.18 (d, J = 8.0 Hz, 1H), 6.81 (d, J = 5.1 Hz, 1H), 5.73 (s, 1H), 4.77-4.65 (m, 1H), 4.27 (d, J = 10.6 Hz, 1H), 3.67-3.52 (m, 1H), 3.02- 2.94 (m, 1H), 2.88 (p, J = 6.7 Hz, 1H), 1.88 (s, 3H), 1.77 (s, 3H), 1.49 (s, 3H), 1.15 (dd, J = 18.9, 6.8 Hz, 6H). | m/z 487.4 (M + H)⁺ (ES⁺) |
| 13 | 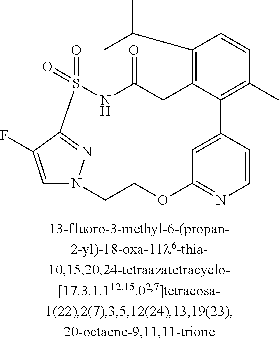 [SMILES: Cc1ccc(C(C)C)c2c1-c1ccnc(c1)OCCn1cc(F)c(n1)S(=O)(=O)NC(=O)C2] | C13 | ¹H NMR (DMSO-d₆) δ 12.22 (s, 1H), 8.26 (s, 2H), 7.25 (d, J = 8.0 Hz, 1H), 7.19 (d, J = 8.0 Hz, 1H), 6.85 (d, J = 5.0 Hz, 1H), 5.96 (s, 1H), 5.04 (s, 1H), 4.60-4.39 (m, 3H), 3.70-3.56 (m, 1H), 3.03- 2.92 (m, 1H), 2.83 (p, J = 6.9 Hz, 1H), 1.91 (s, 3H), 1.15 (dd, J = 6.9, 1.7 Hz, 6H). | m/z 459.3 (M + H)⁺ (ES⁺) |
| 14 | 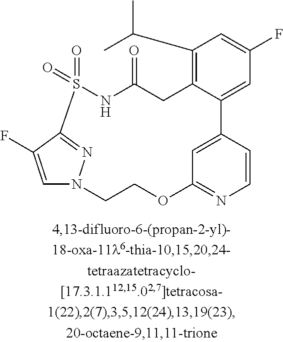 [SMILES: CC(C)c1cc(F)cc2c1CC(=O)NS(=O)(=O)c1nn(cc1F)CCOc1cc-2ccn1] | C14 | ¹H NMR (DMSO-d₆) δ 12.30 (s, 1H), 8.25 (d, J = 5.2 Hz, 2H), 7.21 (dd, J = 10.6, 2.8 Hz, 1H), 6.94 (d, J = 5.2 Hz, 1H), 6.88 (dd, J = 8.6, 2.7 Hz, 1H), 6.09 (s, 1H), 4.75 (s, 2H), 4.48 (s, 2H), 3.39 (s, 2H), 2.97-2.88 (m, 1H), 1.18 (d, J = 6.8 Hz, 6H). | m/z 463.7 (M + H)⁺ (ES⁺) |
| 15 | 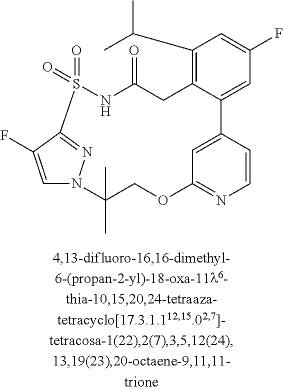 [SMILES: CC(C)c1cc(F)cc2c1CC(=O)NS(=O)(=O)c1nn(cc1F)C(C)(C)COc1cc-2ccn1] | C15 | ¹H NMR (DMSO-d₆) δ 12.15 (s, 1H), 8.28 (s, 1H), 8.25 (d, J = 5.2 Hz, 1H), 7.20 (dd, J = 10.7, 2.8 Hz, 1H), 6.95-6.90 (m, 1H), 6.84 (dd, J = 8.6, 2.7 Hz, 1H), 5.83 (s, 1H), 4.52 (s, 2H), 3.40 (s, 1H), 3.18 (d, J = 3.5 Hz, 1H), 2.98 (p, J = 6.7 Hz, 1H), 1.63 (s, 6H), 1.17 (d, J = 6.8 Hz, 6H). | m/z 491.3 (M + H)⁺ (ES⁺) |
| 19 | 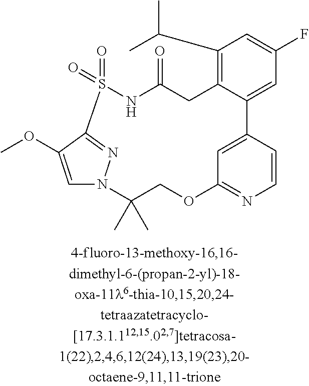 [SMILES: COc1cn2nc1S(=O)(=O)NC(=O)Cc1c(cc(F)cc1C(C)C)-c1ccnc(c1)OCC2(C)C] | C19 | ¹H NMR (DMSO-d₆) δ 11.92 (s, 1H), 8.21 (d, J = 5.2 Hz, 1H), 7.87 (s, 1H), 7.19 (d, J = 10.5 Hz, 1H), 6.95-6.74 (m, 2H), 5.99 (s, 1H), 4.54 (s, 2H), 3.70 (s, 3H), 2.96-2.85 (m, 1H), 1.60 (s, 6H), 1.16 (d, J = 6.8 Hz, 6H). Peak containing two protons obscured by water peak at 3.31 ppm. | m/z 503.4 (M + H)⁺ (ES⁺) |
| 21 | 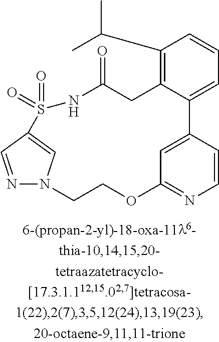 [SMILES: CC(C)c1cccc2c1CC(=O)NS(=O)(=O)c1cnn(c1)CCOc1cc-2ccn1] | C21 | ¹H NMR (DMSO-d₆) δ 11.77 (s, 1H), 8.10 (s, 1H), 7.95 (d, J = 5.1 Hz, 1H), 7.71 (s, 1H), 7.40-7.28 (m, 2H), 6.98 (dd, J = 7.3, 1.5 Hz, 1H), 6.81 (d, J = 5.2 Hz, 1H), 6.40 (s, 1H), 4.78 (s, 2H), 4.51 (t, J = 4.9 Hz, 2H), 3.52 (s, 2H), 3.07-2.92 (m, 1H), 1.18 (d, J = 6.8 Hz, 6H). | — |
| 22 | 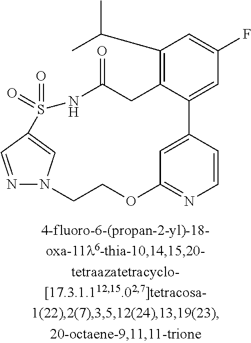 [SMILES: CC(C)c1cc(F)cc2c1CC(=O)NS(=O)(=O)c1cnn(c1)CCOc1cc-2ccn1] | C22 | ¹H NMR (DMSO-d₆) δ 8.09 (s, 1H), 7.96 (d, J = 5.2 Hz, 1H), 7.70 (s, 1H), 7.19 (dd, J = 10.6, 2.8 Hz, 1H), 6.87 (dd, J = 8.7, 2.7 Hz, 1H), 6.82 (d, J = 5.2 Hz, 1H), 6.41 (s, 1H), 4.79 (s, 2H), 4.51 (t, J = 4.8 Hz, 2H), 3.50 (s, 2H), 3.06-2.94 (m, 1H), 1.17 (d, J = 6.8 Hz, 6H). One exchangeable proton not observed. | m/z 445.5 (M + H)⁺ (ES⁺) |
| 24 | 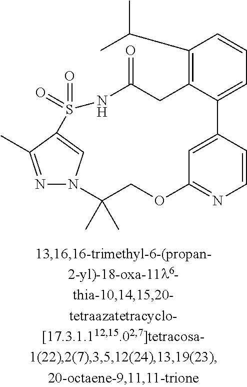 [SMILES: Cc1nn2cc1S(=O)(=O)NC(=O)Cc1c(cccc1C(C)C)-c1ccnc(c1)OCC2(C)C] | C24 | ¹H NMR (DMSO-d₆) δ 11.78 (br s, 1H), 7.95 (d, J = 5.1 Hz, 1H), 7.88 (br s, 1H), 7.38-7.27 (m, 2H), 6.93 (dd, J = 7.3, 1.5 Hz, 1H), 6.76 (br s, 1H), 6.37 (br s, 1H), 4.61 (br s, 2H), 3.50 (br s, 2H), 3.09- 2.92 (m, 1H), 2.17 (s, 3H), 1.63 (s, 6H), 1.18 (d, J = 6.7 Hz, 6H). | m/z 469.5 (M + H)⁺ (ES⁺) |

### Example 6: 16-fluoro-19,19-dimethyl-21-oxa-14λ⁶-thia-13,18,23,27-tetraazapentacyclo[20.3.1.1^{15,18}.0^{2,10}.0^{5,9}]heptacosa-1(25),2,4,9,15(27),16,22(26),23-octaene-12,14,14-trione

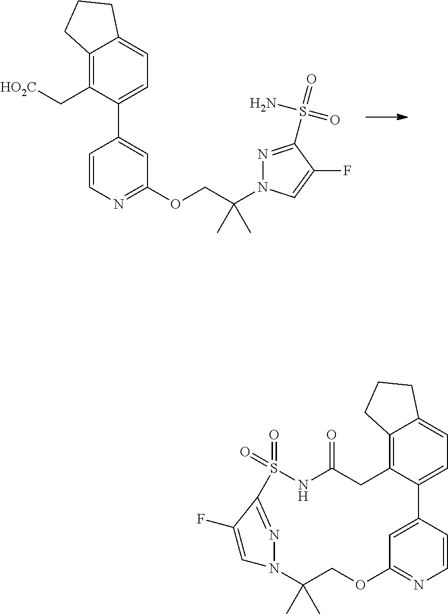 [SMILES: CC(C)(COc1cc(-c2ccc3c(c2CC(=O)O)CCC3)ccn1)n1cc(F)c(S(N)(=O)=O)n1 ; CC1(C)COc2cc(ccn2)-c2ccc3c(c2CC(=O)NS(=O)(=O)c2nn1cc2F)CCC3]

2-(5-(2-(2-(4-Fluoro-3-sulfamoyl-1H-pyrazol-1-yl)-2-methylpropoxy)pyridin-4-yl)-2,3-dihydro-1H-inden-4-yl)acetic acid (Intermediate C6) (45 mg, 0.092 mmol) was dissolved in NMP (2 mL), to which was added HATU (52 mg, 0.137 mmol) and DIPEA (48 μL, 0.275 mmol). The reaction was stirred at RT for 18 h. The mixture was purified by basic prep HPLC (20-50% MeCN in water) to afford the title compound (8 mg, 18%) as a white solid.

LCMS m/z 471.4 (M+H)⁺ (ES⁺).

¹H NMR (DMSO-d₆) δ 12.12 (s, 1H), 8.28 (d, J=4.2 Hz, 1H), 8.20 (d, J=5.1 Hz, 1H), 7.19 (d, J=7.5 Hz, 1H), 6.90 (d, J=7.5 Hz, 1H), 6.86 (dd, J=5.2, 1.4 Hz, 1H), 5.85 (s, 1H), 4.53 (s, 2H), 3.38 (s, 2H), 2.93 (t, J=7.4 Hz, 2H), 2.80 (t, J=7.5 Hz, 2H), 2.03 (p, J=7.5 Hz, 2H), 1.63 (s, 6H).

### Example 17: 17-(2-hydroxypropan-2-yl)-22-oxa-14λ⁶-thia-13,24-diazapentacyclo-[21.3.1.1^{15,19}.0^{2,10}.0^{5,9}]octacosa-1(26),2,4,9,15,17,19(28),23(27),24-nonaene-12,14,14-trione

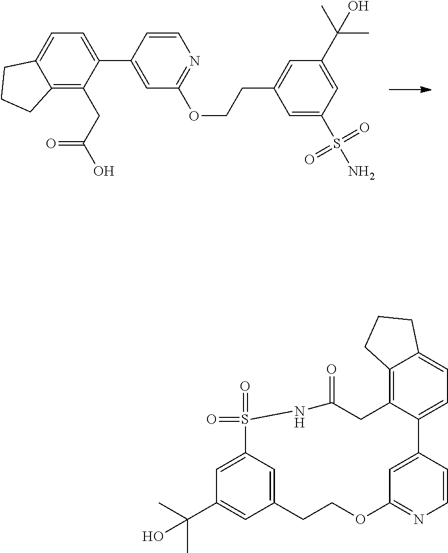

CDI (57 mg, 0.352 mmol) was dissolved in MeCN (10 mL), to which was added 2-(5-(2-(3-(2-hydroxypropan-2-yl)-5-sulfamoylphenethoxy)pyridin-4-yl)-2,3-dihydro-1H-inden-4-yl)acetic acid (Intermediate C17) (192 mg, 0.377 mmol). The mixture was stirred at RT for 1 h. DBU (53 μL, 0.355 mmol) in MeCN (10 mL) was then added and the mixture was stirred at RT for 24 h. The reaction was concentrated in vacuo and the resulting residue purified by acidic prep HPLC (35-65% MeCN in water) to afford the title compound (6 mg, 3%) as a white solid.

LCMS m/z 493.4 (M+H)⁺ (ES⁺).

¹H NMR (CDCl₃) δ 7.97-7.87 (m, 2H), 7.85-7.73 (m, 2H), 7.47 (s, 1H), 7.29 (s, 1H), 7.06 (d, J=7.8 Hz, 1H), 6.69 (d, J=5.2 Hz, 1H), 6.46 (br s, 1H), 5.00-4.79 (m, 2H), 3.77-3.65 (m, 2H), 3.14 (t, J=5.6 Hz, 2H), 3.04 (t, J=7.5 Hz, 2H), 2.93 (t, J=7.5 Hz, 2H), 2.18 (p, J=7.5 Hz, 2H), 1.60 (s, 6H). One exchangeable proton not observed.

The following examples ‘Ex’ were synthesised following the general procedure for Example 17, from the intermediate compounds indicated in the ‘From’ column:

| Ex | Structure | From | ¹H NMR | LCMS |
|---|---|---|---|---|
| 16 | 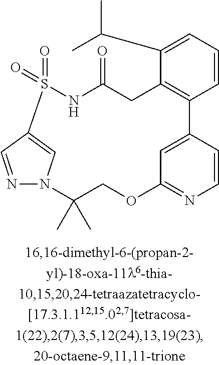 [SMILES: CC(C)c1cccc2c1CC(=O)NS(=O)(=O)c1cnn(c1)C(C)(C)COc1cc-2ccn1] | C16 | ¹H NMR (DMSO-d₆) δ 11.75 (s, 1H), 8.03 (s, 1H), 7.92 (d, J = 5.1 Hz, 1H), 7.67 (s, 1H), 7.36 (dd, J = 8.0, 1.5 Hz, 1H), 7.31 (t, J = 7.6 Hz, 1H), 6.95 (dd, J = 7.3, 1.5 Hz, 1H), 6.80 (s, 1H), 6.37 (s, 1H), 4.57 (s, 2H), 3.59- 3.50 (m, 2H), 3.07-2.98 (m, 1H), 1.69 (s, 6H), 1.18 (d, J = 6.8 Hz, 6H). | m/z 455.4 (M + H)⁺ (ES⁺) |
| 18 | 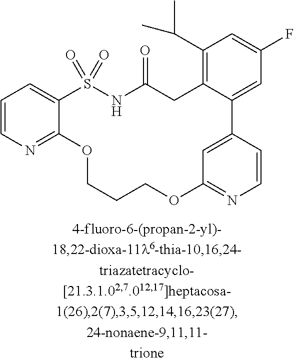 [SMILES: CC(C)c1cc(F)cc2c1CC(=O)NS(=O)(=O)c1cccnc1OCCCOc1cc-2ccn1] | C18 | ¹H NMR (DMSO-d₆) δ 12.38 (s, 1H), 8.43 (s, 1H), 8.27 (s, 1H), 8.25- 8.19 (m, 1H), 7.20 (dd, J = 10.6, 2.8 Hz, 1H), 7.14 (s, 1H), 6.91-6.82 (m, 3H), 4.66-4.38 (m, 4H), 3.32 (s, 2H), 3.03 (s, 1H), 2.34-2.18 (m, 2H), 1.18 (d, J = 5.9 Hz, 6H). | m/z 486.0 (M + H)⁺ (ES⁺) |
| 20 | 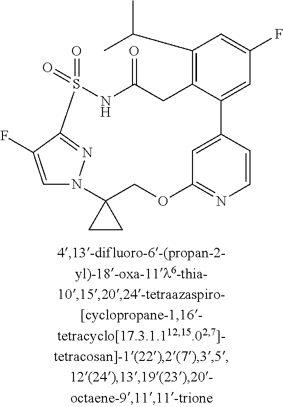 [SMILES: CC(C)c1cc(F)cc2c1CC(=O)NS(=O)(=O)c1nn(cc1F)C1(CC1)COc1cc-2ccn1] | C20 | ¹H NMR (DMSO-d₆) δ 12.35 (s, 1H), 8.38 (d, J = 4.3 Hz, 1H), 8.21 (d, J = 5.1 Hz, 1H), 7.22 (dd, J = 10.7, 2.8 Hz, 1H), 6.97- 6.90 (m, 1H), 6.88 (dd, J = 8.7, 2.7 Hz, 1H), 6.12 (s, 1H), 4.67 (br s, 2H), 3.43 (br s, 2H), 3.02- 2.81 (m, 1H), 1.42-1.30 (m, 2H), 1.29-1.20 (m, 2H), 1.17 (d, J = 6.7 Hz, 6H). | m/z 489.5 (M + H)⁺ (ES⁺) |
| 23 | 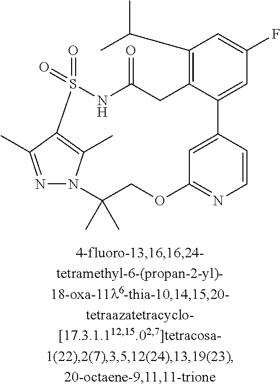 [SMILES: Cc1nn2c(C)c1S(=O)(=O)NC(=O)Cc1c(cc(F)cc1C(C)C)-c1ccnc(c1)OCC2(C)C] | C23 | ¹H NMR (DMSO-d₆) δ 8.11 (d, J = 5.1 Hz, 1H), 7.12 (dd, J = 10.9, 2.8 Hz, 1H), 6.82-6.79 (m, 1H), 6.79-6.72 (m, 1H), 6.18 (s, 1H), 4.70-4.58 (m, 2H), 3.42 (d, J = 18.4 Hz, 1H), 3.33 (d, J = 18.3 Hz, 1H), 2.44 (s, 3H), 2.21 (s, 3H), 1.80 (s, 3H), 1.72 (s, 3H), 1.24-1.18 (m, 6H). CH of ⁱPr masked by water peak and one exchangeable proton not observed. | m/z 501.0 (M + H)⁺ (ES⁺) |
| 25 | 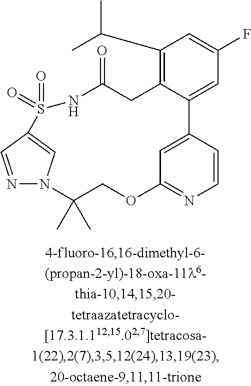 [SMILES: CC(C)c1cc(F)cc2c1CC(=O)NS(=O)(=O)c1cnn(c1)C(C)(C)COc1cc-2ccn1] | C25 | ¹H NMR (DMSO-d₆) δ 11.75 (br s, 1H), 8.03 (s, 1H), 7.93 (d, J = 5.1 Hz, 1H), 7.68 (s, 1H), 7.18 (dd, J = 10.6, 2.8 Hz, 1H), 6.90-6.72 (m, 2H), 6.37 (br s, 1H), 4.60 (br s, 2H), 3.53 (br s, 2H), 3.12-2.93 (m, 1H), 1.69 (s, 6H), 1.17 (d, J = 6.8 Hz, 6H). | m/z 473.5 (M + H)⁺ (ES⁺) |
| 26 | 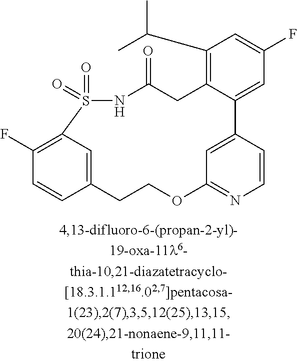 [SMILES: CC(C)c1cc(F)cc2c1CC(=O)NS(=O)(=O)c1cc(ccc1F)CCOc1cc-2ccn1] | C26 | ¹H NMR (DMSO-d₆) δ 8.25 (d, J = 5.1 Hz, 1H), 7.57 (s, 1H), 7.30 (dd, J = 10.3, 8.4 Hz, 1H), 7.18 (dd, J = 10.7, 2.8 Hz, 1H), 7.02-6.97 (m, 1H), 6.94-6.84 (m, 2H), 6.52 (s, 1H), 4.66 (s, 2H), 3.16-2.95 (m, 2H), 2.91 (t, J = 5.5 Hz, 2H), 1.92 (p, J = 5.8 Hz, 1H), 1.20 (d, J = 6.7 Hz, 6H). One exchangeable proton not observed. | m/z 473.2 (M + H)⁺ (ES⁺) m/z 471.3 (M − H)⁻ (ES⁻) |
| 27 | 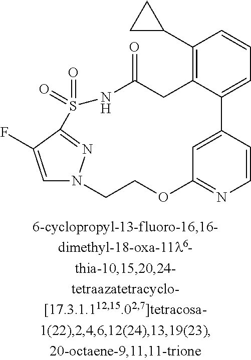 [SMILES: O=C1Cc2c(cccc2C2CC2)-c2ccnc(c2)OCCn2cc(F)c(n2)S(=O)(=O)N1] | C27 | ¹H NMR (DMSO-d₆) δ 12.20 (s, 1H), 8.28-8.19 (m, 2H), 7.23 (t, J = 7.7 Hz, 1H), 7.09 (d, J = 7.7 Hz, 1H), 6.96 (dd, J = 7.5, 1.3 Hz, 1H), 6.88 (dd, J = 5.1, 1.5 Hz, 1H), 5.90 (br s, 1H), 4.51 (br s, 2H), 3.56 (br s, 2H), 1.81 (m, 1H), 1.61 (s, 6H), 0.96-0.85 (m, 2H), 0.61 (d, J = 5.1 Hz, 2H). | m/z 471.1 (M + H)⁺ (ES⁺) |
| 28 | 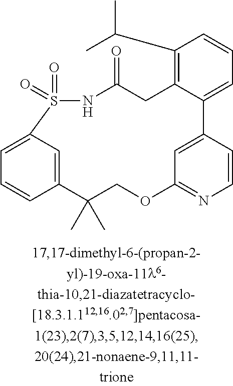 [SMILES: CC(C)c1cccc2c1CC(=O)NS(=O)(=O)c1cccc(c1)C(C)(C)COc1cc-2ccn1] | C28 | ¹H NMR (DMSO-d₆) δ 11.95 (s, 1H), 8.13-8.07 (m, 1H), 7.78 (d, J = 7.9 Hz, 1H), 7.70-7.66 (m, 1H), 7.57 (s, 1H), 7.50 (t, J = 7.9 Hz, 1H), 7.35 (dd, J = 7.9, 1.5 Hz, 1H), 7.28 (t, J = 7.1 Hz, 1H), 6.93 (dd, J = 7.4, 1.4 Hz, 1H), 6.79 (d, J = 4.9 Hz, 1H), 5.90 (s, 1H), 4.48 (s, 2H), 3.45 (s, 2H), 2.99-2.85 (m, 1H), 1.42 (s, 6H), 1.17 (d, J = 6.8 Hz, 6H). | m/z 465.4 (M + H)⁺ (ES⁺) 463.4 (M − H)⁻ (ES⁻) |
| 29 | 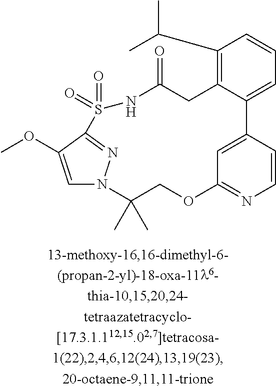 [SMILES: COc1cn2nc1S(=O)(=O)NC(=O)Cc1c(cccc1C(C)C)-c1ccnc(c1)OCC2(C)C] | C29 | ¹H NMR (DMSO-d₆) δ 11.94 (s, 1H), 8.20 (d, J = 5.3 Hz, 1H), 7.88 (s, 1H), 7.36 (d, J = 7.8 Hz, 1H), 7.30 (t, J = 7.7 Hz, 1H), 6.97 (d, J = 7.3 Hz, 1H), 6.86 (d, J = 4.7 Hz, 1H), 6.01 (s, 1H), 4.55 (s, 2H), 3.70 (s, 3H), 2.93-2.84 (m, 1H), 1.60 (s, 6H), 1.17 (d, J = 6.8 Hz, 6H). Two aliphatic protons obscured by water peak. | m/z 485.4 (M + H)⁺ (ES⁺) |
| 33 | 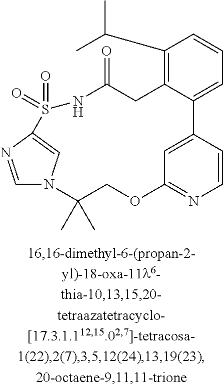 [SMILES: CC(C)c1cccc2c1CC(=O)NS(=O)(=O)c1cn(cn1)C(C)(C)COc1cc-2ccn1] | C33 | ¹H NMR (DMSO-d₆) δ 11.73 (s, 1H), 8.11 (s, 1H), 7.92 (d, J = 5.2 Hz, 1H), 7.61 (s, 1H), 7.35 (d, J = 7.8 Hz, 1H), 7.31 (t, J = 7.6 Hz, 1H), 6.93 (d, J = 7.2 Hz, 1H), 6.85-6.67 (m, 1H), 6.41 (s, 1H), 3.54 (s, 2H), 3.31 (s, 2H), 3.00-2.89 (m, 1H), 1.68 (s, 6H), 1.17 (d, J = 6.8 Hz, 6H). | m/z 455.4 (M + H)⁺ (ES⁺) |
| 37 | 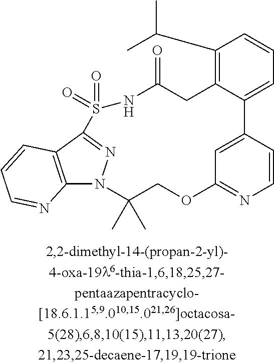 [SMILES: CC(C)c1cccc2c1CC(=O)NS(=O)(=O)c1nn(c3ncccc13)C(C)(C)COc1cc-2ccn1] | C37 | ¹H NMR (DMSO-d₆) δ 12.51 (s, 1H), 8.79-8.68 (m, 1H), 8.34 (d, J = 8.5 Hz, 1H), 7.86 (d, J = 42.1 Hz, 1H), 7.46 (dd, J = 8.2, 4.5 Hz, 1H), 7.35 (dd, J = 7.8, 1.4 Hz, 1H), 7.28 (t, J = 7.6 Hz, 1H), 6.91 (d, J = 7.4 Hz, 1H), 6.77 (s, 1H), 5.89 (s, 1H), 4.97 (s, 2H), 3.50 (d, J = 25.3 Hz, 2H), 2.94 (d, J = 7.9 Hz, 1H), 1.96 (d, J = 27.3 Hz, 6H), 1.31-1.13 (m, 6H). | m/z 528.4 (M + Na)⁺ (ES⁺) |

### Example 30: 14-cyclopropyl-4-fluoro-6-(propan-2-yl)-19-oxa-11λ⁶-thia-10,21-diazatetracyclo[18.3.1.11^{2,16}.0^{2,7}]pentacosa-1(23),2(7),3,5,12(25),13,15,20(24),21-nonaene-9,11,11-trione

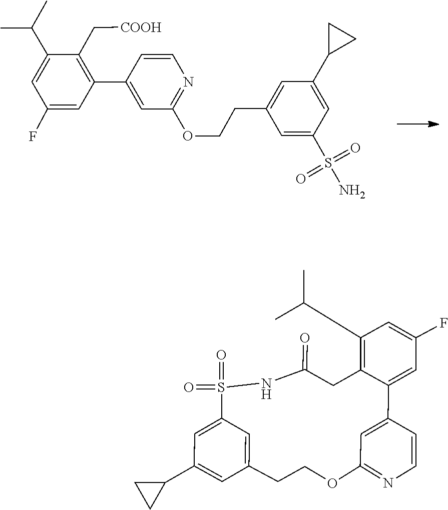 [SMILES: CC(C)c1cc(F)cc(-c2ccnc(OCCc3cc(C4CC4)cc(S(N)(=O)=O)c3)c2)c1CC(=O)O ; CC(C)c1cc(F)cc2c1CC(=O)NS(=O)(=O)c1cc(cc(C3CC3)c1)CCOc1cc-2ccn1]

2-(2-(2-(3-cyclopropyl-5-sulfamnoylphenethoxy)pyridin-4-yl)-4-fluoro-6-isopropyl-phenyl)acetic acid (Intermediate C30) (150 mg, 293 μmol) was dissolved in DMF (2 mL), to which was added DMAP (107 mg, 878 μmol) and DIC (111 mg, 0.14 mL, 878 μmol). The mixture was stirred at RT for 48 h. The reaction was diluted with 1 M aq HCl (15 mL) and extracted with EtOAc (25 mL). The organic layer was washed with brine (2×25 mL), dried using a phase separator and concentrated in vacuo. The crude product was purified by FC (0-100% EtOAc/isohexane) to afford the title compound (30 mg, 20%) as a white solid.

LCMS m/z 495.5 (M+H)⁺ (ES⁺); 493.4 (M−H)⁻ (ES⁻).

¹H NMR (DMSO-d₆) δ 11.91 (br s, 1H), 8.22 (d, J=5.1 Hz, 1H), 7.43-7.34 (m, 2H), 7.20 (dd, J=10.6, 2.8 Hz, 1H), 6.96-6.77 (m, 3H), 6.00 (s, 1H), 4.71 (br s, 2H), 3.52 (br s, 2H), 3.06 (t, J=5.8 Hz, 2H), 2.99-2.88 (m, 1H), 2.09-2.01 (m, 1H), 1.17 (d, J=6.8 Hz, 6H), 1.06-1.00 (m, 2H), 0.78-0.70 (m, 2H).

The following examples ‘Ex.’ were synthesised following the general procedure for Example 30, from the intermediate compounds indicated in the ‘From’ column:

| Ex. | Structure | From | ¹H NMR | LCMS |
|---|---|---|---|---|
| 31 | 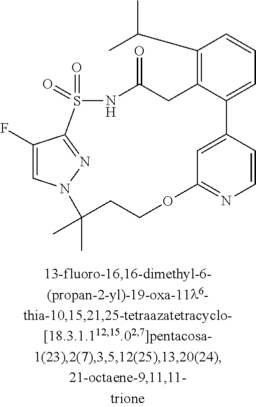 [SMILES: CC(C)c1cccc2c1CC(=O)NS(=O)(=O)c1nn(cc1F)C(C)(C)CCOc1cc-2ccn1] | C31 | ¹H NMR (DMSO-d₆) δ 12.28 (s, 1H), 8.42 (s, 1H), 8.16 (d, J = 5.1 Hz, 1H), 7.39 (d, J = 7.8 Hz, 1H), 7-33 (t, J = 7.6 Hz, 1H), 6.96 (dd, J = 7.5, 1.4 Hz, 1H), 6.83 (dd, J = 5.2, 1.4 Hz, 1H), 5.83 (s, 1H), 4.28 (s, 2H), 2.93 (p, J = 6.9 Hz, 1H), 2.34 (s, 2H), 1.60 (s, 6H), 1.57-1.49 (m, 1H), 1.18 (d, J = 6.7 Hz, 6H), 0.95 (t, J = 7.3 Hz, 1H). | m/z 487.4 (M + H)⁺ (ES⁺) 485.3 (M − H)⁻ (ES⁻) |
| 32 | 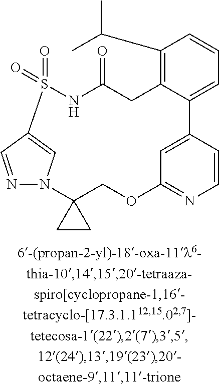 [SMILES: CC(C)c1cccc2c1CC(=O)NS(=O)(=O)c1cnn(c1)C1(CC1)COc1cc-2ccn1] | C32 | ¹H NMR (DMSO-d₆) δ 11.81 (s, 1H), 8.01 (br s, 1H), 7.92 (d, J = 5.1 Hz, 1H), 7.71 (s, 1H), 7.39- 7.30 (m, 2H), 6.99 (dd, J = 7.2, 1.5 Hz, 1H), 6.82 (br s, 1H), 6.47 (s, 1H), 4.70 (br s, 2H), 3.58 (s, 2H), 3.09-2.95 (m, 1H), 1.36 (app br s, 2H), 1.26 (app s, 2H), 1.18 (d, J = 6.7 Hz, 6H). | m/z 453.4 (M + H)⁺ (ES⁺) |
| 34 | 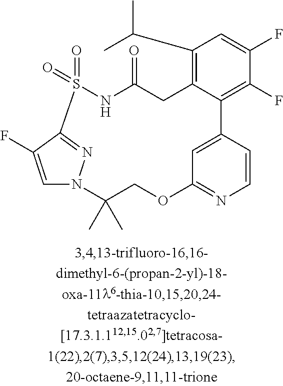 [SMILES: CC(C)c1cc(F)c(F)c2c1CC(=O)NS(=O)(=O)c1nn(cc1F)C(C)(C)COc1cc-2ccn1] | C34 | ¹H NMR (DMSO-d₆) δ 12.14 (s, 1H), 8.31 (d, J = 5.1 Hz, 1H), 8.28-8.23 (m, 1H), 7.43 (dd, J = 12.3, 8.1 Hz, 1H), 6.98 (d, J = 5.1 Hz, 1H), 5.85 (s, 1H), 4.76 (s, 1H), 4.33- 4.24 (m, 1H), 3.78-3.67 (m, 1H), 3.09-2.92 (m, 2H), 1.77 (s, 3H), 1.48 (s, 3H), 1.15 (s, 6H). | m/z 508.9 (M + H)⁺ (ES⁺) 506.9 (M − H)⁻ (ES⁻) |
| 35 | 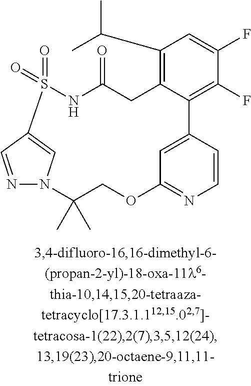 [SMILES: CC(C)c1cc(F)c(F)c2c1CC(=O)NS(=O)(=O)c1cnn(c1)C(C)(C)COc1cc-2ccn1] | C35 | ¹H NMR (DMSO-d₆) δ 11.77 (s, 1H), 8.08 (s, 1H), 8.05-8.00 (m, 1H), 7.67 (s, 1H), 747-7.37 (m, 1H), 6.85 (s, 1H), 6.42 (s, 1H), 5.25 (s, 1H), 4.03-3-89 (m, 1H), 3.81- 3.67 (m, 1H), 3.16 (s, 1H), 3.06-2.95 (m, 1H), 1.77 (s, 3H), 1.63 (s, 3H), 1.24-1.06 (m, 6H). | m/z 491.4 (M + H)⁺ (ES⁺) 489.2 (M − H)⁻ (ES⁻) |
| 36 | 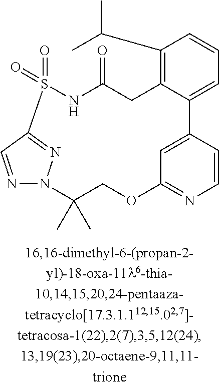 [SMILES: CC(C)c1cccc2c1CC(=O)NS(=O)(=O)c1cnn(n1)C(C)(C)COc1cc-2ccn1] | C36 | ¹H NMR (DMSO-d₆) δ 12.31 (br s, 1H), 8.21 (d, J = 5.2 Hz, 1H), 7.40- 7.33 (m, 2H), 7.29 (t, J = 7.7 Hz, 1H), 6.91 (dd, J = 11.1, 6.2 Hz, 2H), 5.77 (br s, 1H), 4.66 (br s, 2H), 3.45 (br s, 2H), 2.98 (sept, J = 6.8 Hz, 1H), 1.76 (s, 6H), 1.17 (d, J = 6.7 Hz, 6H). | m/z 456.4 (M + H)⁺ (ES⁺) 454.3 (M − H)⁻ (ES⁻) |
| 38 | 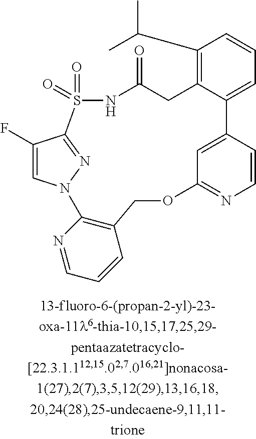 [SMILES: CC(C)c1cccc2c1CC(=O)NS(=O)(=O)c1nn(cc1F)-c1ncccc1COc1cc-2ccn1] | C38 | ¹H NMR (DMSO-d₆) δ 12.41 (s, 1H), 8.72 (s, 1H), 8.60 (d, J = 4.7 Hz, 1H), 8.32-8.26 (m, 2H), 7.66 (dd, J = 7.7, 4.8 Hz, 1H), 7.41-7.35 (m, 1H), 7.31 (t, J = 7.6 Hz, 1H), 7.02-6.91 (m, 2H), 5.93 (br s, 1H), 5.58 (br s, 2H), 3.49, (br s, 2H) 3.08 (br s, 1H), 1.21 (d, J = 6.8 Hz, 6H). | m/z 508.4 (M + H)⁺ (ES⁺) |
| 39 | 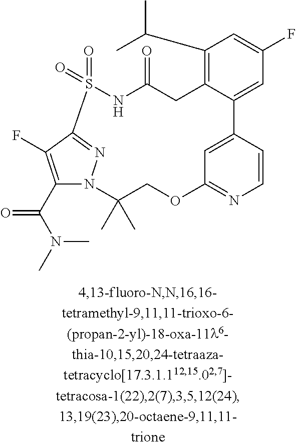 [SMILES: CC(C)c1cc(F)cc2c1CC(=O)NS(=O)(=O)c1nn(c(C(=O)N(C)C)c1F)C(C)(C)COc1cc-2ccn1] | C39 | ¹H NMR (DMSO-d₆) δ 12.35 (s, 1H), 8.27 (d, J = 5.1 Hz, 1H), 7.19 (dd, J = 10.7, 2.8 Hz, 1H), 6.94 (dd, J = 5.1, 1.4 Hz, 1H), 6.86 (dd, J = 8.5, 2.8 Hz, 1H), 5.93 (s, 1H), 4.72 (s, 2H), 4.42 (s, 2H), 3.69- 3.55 (m, 1H), 3.02 (d, J = 10.7 Hz, 6H), 1.17 (d, J = 6.7 Hz, 6H), 1.00 (d, J = 6.5 Hz, 6H). | m/z 562.4 (M + H)⁺ (ES⁺) |
| 40 | 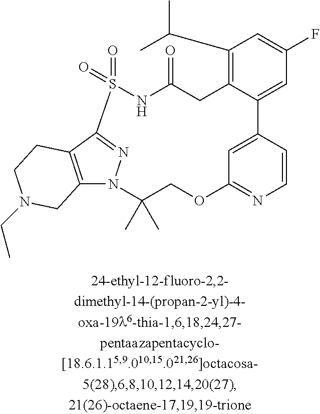 [SMILES: CCN1CCc2c3nn(c2C1)C(C)(C)COc1cc(ccn1)-c1cc(F)cc(C(C)C)c1CC(=O)NS3(=O)=O] | C40 | ¹H NMR (DMSO-d₆) δ 8.23 (s, 1H), 7.19 (dd, J = 10.7, 2.8 Hz, 1H), 6.89 (s, 1H), 6.84 (dd, J = 8.6, 2.7 Hz, 1H), 5.93 (s, 1H), 4.58 (br s, 2H), 3.39 (s, 2H), 2.96 (br s, 2H), 2.83 (br s, 2H), 1.66 (br s, 6H), 1.20 (s, 3H), 1.16 (d, J = 6.7 Hz, 6H). Five aliphatic protons overlapped with DMSO and water peaks; one exchangeable proton not observed. | m/z 556.4 (M + H)⁺ (ES⁺) |
| 41 | 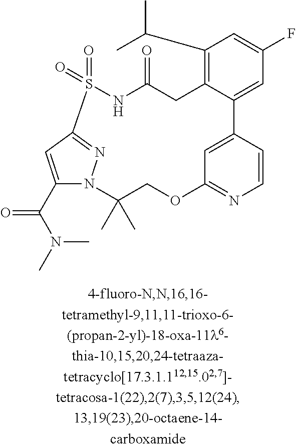 [SMILES: CC(C)c1cc(F)cc2c1CC(=O)NS(=O)(=O)c1cc(C(=O)N(C)C)n(n1)C(C)(C)COc1cc-2ccn1] | C41 | ¹H NMR (DMSO-d₆) δ 12.17 (br s, 1H), 8.27 (d, J = 5.2 Hz, 1H), 7.18 (dd, J = 10.8, 2.8 Hz, 1H), 6.92 (dd, J = 5.2, 1.5 Hz, 1H), 6.84 (dd, J = 8.6, 2.8 Hz, 1H), 6.76 (s, 1H), 5.92 (s, 1H), 4.57 (br s, 2H), 3.06-2.90 (m, 7H), 1.62 (s, 6H), 1.17 (d, J = 6.8 Hz, 6H). One CH₂ obscured by water peak. | m/z 544.4 (M + H)⁺ (ES⁺) 542.3 (M − H)⁻ (ES⁻) |
| 42 | 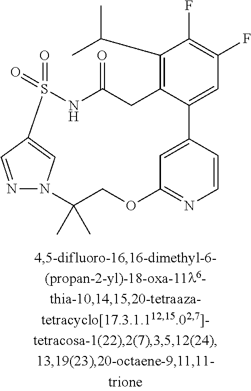 [SMILES: CC(C)c1c(F)c(F)cc2c1CC(=O)NS(=O)(=O)c1cnn(c1)C(C)(C)COc1cc-2ccn1] | C42 | ¹H NMR (DMSO-d₆) δ 11.76 (s, 1H), 8.11 (s, 1H), 7.99 (d, J = 5.2 Hz, 1H), 7.69 (s, 1H), 7.13 (dd, J = 10.5, 7.8 Hz, 1H), 6.83 (s, 1H), 6.32 (s, 1H), 4.55 (s, 2H), 3.49 (s, 2H), 3.17- 3.06 (m, 1H), 1.69 (s, 6H), 1.39-1.21 (m, 6H). | m/z 491.4 (M + H)⁺ (ES⁺) 489.4 (M − H)⁻ (ES⁻) |

### Example 41: 6-(propan-2-yl)-22-oxa-11λ⁶-thia-10,15,16,24-tetraazatetracyclo-[21.3.1.0^{2,7}.0^{12,16}]heptacosa-1(26),2(7),3,5,12,14,23(27),24-octaene-9,11,11-trione

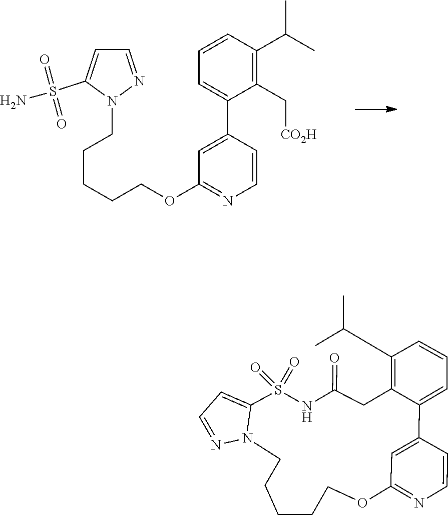 [SMILES: CC(C)c1cccc(-c2ccnc(OCCCCCn3nccc3S(N)(=O)=O)c2)c1CC(=O)O ; CC(C)c1cccc2c1CC(=O)NS(=O)(=O)c1ccnn1CCCCCOc1cc-2ccn1]

To a solution of 2-(2-isopropyl-6-(2-((5-(5-sulfamoyl-1H-pyrazol-1-yl)pentyl)oxy)-pyridin-4-yl)phenyl)acetic acid (Intermediate C43) (330 mg, 678 μmol) in THF (20 mL) was added TEA (275 mg, 2.71 mmol) and BOP (600 mg, 1.36 mmol) at 20° C. The mixture was stirred for 0.5 h. Then DMAP (331 mg, 2.71 mmol) was added into the mixture and the resulting mixture was stirred at 20° C. for 1 h. The reaction mixture was concentrated in vacuum. The residue was purified by prep-HPLC (column: Waters Xbridge C18, 150 mm×25 mm×5 μm; mobile phase: [A: water (10 mM NH₄HCO₃), B: MeCN]; B %: 15%-45%, 9 min) to give the title compound (42.8 mg, 13.4%) as a white solid.

LCMS: m/z 469.1 (M+H)⁺ (ES⁺).

¹H NMR (CD₃OD) δ 8.12 (d, 1H), 7.47 (d, 1H), 7.37 (d, 1H), 7.29 (t, 1H), 7.04 (d, 1H), 6.86 (d, 1H), 6.82 (dd, 1H), 6.61 (t, 1H), 4.44 (t, 2H), 4.37 (t, 2H), 3.55 (s, 2H), 3.14-3.09 (m, 1H), 1.98-1.95 (m, 2H), 1.75-1.72 (m, 2H), 1.57-1.53 (m, 2H), 1.27 (d, 6H). One exchangeable proton not observed.

### Example 44: 6-(propan-2-yl)-18-oxa-11λ⁶-thia-10,15,20,21,24-pentaazatetracyclo-[17.3.1.1^{12,15}.0^{2,7}]tetracosa-1(22),2(7),3,5,12(24),13,19(23),20-octaene-9,11,11-trione

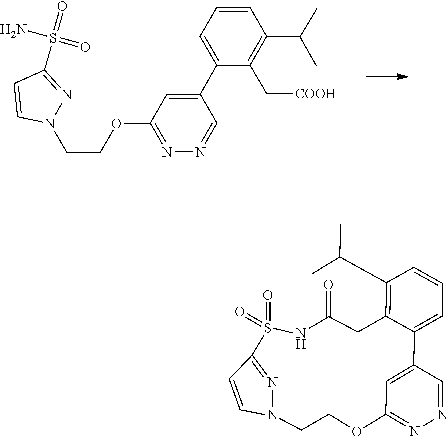 [SMILES: CC(C)c1cccc(-c2cnnc(OCCn3ccc(S(N)(=O)=O)n3)c2)c1CC(=O)O ; CC(C)c1cccc2c1CC(=O)NS(=O)(=O)c1ccn(n1)CCOc1cc-2cnn1]

To a solution of 2-(2-isopropyl-6-(6-(2-(3-sulfamoyl-1H-pyrazol-1-yl)ethoxy)pyridazin-4-yl)phenyl)acetic acid (Intermediate C44) (200 mg, 449 μmol) in THF (15 mL) was added TEA (182 mg, 1.80 mmol) and BOP (398 mg, 898 μmol) at 20° C. The mixture was stirred at 20° C. for 1 h, then DMAP (219 mg, 1.80 mmol) was added and the resulting mixture was stirred at 20° C. for 1 h. The reaction mixture was adjusted to pH 7 with TFA, and then the mixture was concentrated in vacuum. The residue was purified by prep-HPLC (column: Phenomenex Gemini-NX C18, 75 mm×30 mm×3 μm; mobile phase: [A: water (0.1% TFA); B: MeCN]; B %: 38%-48%, 7 min), and then by prep-HPLC (column: Waters Xbridge C18, 150 mm×50 mm×10 μm; mobile phase: [A: water (10 mM NH₄HCO₃); B: MeCN]; B %: 10%-40%, 10 min) to give the title compound (8.82 mg, 4.59% yield, 99.8% purity by HPLC) as a white solid.

LCMS: m/z 428.1 (M+H)⁺ (ES⁺).

¹H NMR (DMSO-d₆+D₂O) δ 8.77 (s, 1H), 7.89 (s, 1H), 7.38 (d, 1H), 7.32 (t, 1H), 7.69 (d, 1H), 6.62 (s, 1H), 6.42 (s, 1H), 4.92 (t, 2H), 4.55 (t, 2H), 3.42 (s, 2H), 3.00-2.90 (m, 1H), 1.15 (d, 6H). One exchangeable proton not observed.

### Example 45: 17-methyl-6-(propan-2-yl)-20-oxa-11λ⁶-thia-10,12,17,22-tetraaza-tetracyclo[19.3.1.1^{12,16}.0^{2,7}]hexacosa-1(24),2(7),3,5,21(25),22-hexaene-9,11,11-trione

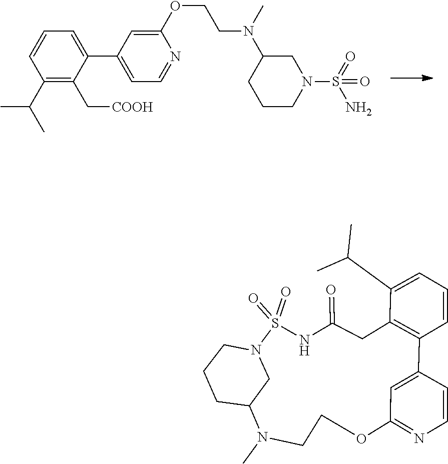 [SMILES: CC(C)c1cccc(-c2ccnc(OCCN(C)C3CCCN(S(N)(=O)=O)C3)c2)c1CC(=O)O ; CC(C)c1cccc2c1CC(=O)NS(=O)(=O)N1CCCC(C1)N(C)CCOc1cc-2ccn1]

To a solution of 2-(2-isopropyl-6-(2-(2-(methyl(1-sulfamoylpiperidin-3-yl)amino)-ethoxy)pyridin-4-yl)phenyl)acetic acid (Intermediate D1) (85 mg, 118 μmol, di-TFA salt) in DCM (40 mL) was added TEA (36 mg, 355 μmol) and BOP (78 mg, 177 μmol). The resulting mixture was stirred at 25° C. for 1 h. To the mixture was added DMAP (43 mg, 355 μmol) and the mixture was stirred at 25° C. for 10 h. The reaction mixture was concentrated in vacuum. The residue was purified by reverse phase flash chromatography (0.1% TFA in H₂O-MeCN) and then by prep-HPLC (column: Waters Xbridge C18, 150 mm×25 mm×5 μm; mobile phase: [A: water (10 mM NH₄HCO₃), B: MeCN]; B %: 18%-48%, 10 min) to give the title compound (39.2 mg, 70.1% yield, 100% purity on LCMS) as a white solid.

LCMS: m/z 473.3 (M+H)⁺ (ES⁺).

¹H NMR (CD₃OD): δ 8.21 (d, 1H), 7.43-7.33 (m, 2H), 7.07 (d, 1H), 6.90 (d, 1H), 6.62 (s, 1H), 4.58-4.39 (m, 2H), 3.91-3.86 (m, 1H), 3.69-3.66 (m, 1H), 3.53-3.48 (m, 2H), 3.17-3.08 (m, 2H), 3.01-2.97 (m, 2H), 2.84 (t, 1H), 2.69 (t, 1H), 2.51 (s, 3H), 2.11-2.04 (m, 1H), 1.85-1.80 (m, 1H), 1.66-1.50 (m, 2H), 1.26 (d, 6H). One exchangeable proton not observed.

### Example 46: 6-isopropyl-16,16-dimethyl-9,11,11-trioxo-18-oxa-11λ⁶-thia-10,14,15,20-tetrazatetra-cyclo[17.3.1.1^{12,15}.0^{2,7}]tetracosa-1(23),2(7),3,5,12(24),13,19,21-octaene-4-carbonitrile

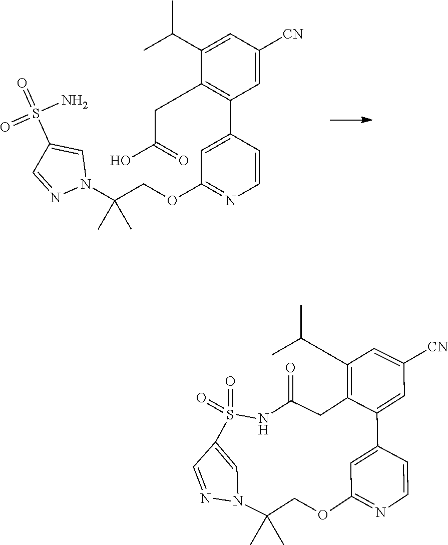 [SMILES: CC(C)c1cc(C#N)cc(-c2ccnc(OCC(C)(C)n3cc(S(N)(=O)=O)cn3)c2)c1CC(=O)O ; CC(C)c1cc(C#N)cc2c1CC(=O)NS(=O)(=O)c1cnn(c1)C(C)(C)COc1cc-2ccn1]

To a solution of 2-(4-cyano-2-isopropyl-6-(2-(2-methyl-2-(4-sulfamoyl-1H-pyrazol-1-yl)propoxy)pyridin-4-yl)phenyl)acetic acid (Intermediate C46) (0.55 g, 1.11 mmol) in DCM (50 mL) was added TEA (447 mg, 4.42 mmol) and BOP (978 mg, 2.21 mmol). The mixture was stirred at 25° C. for 1 h and then DMAP (540 mg, 4.42 mmol) was added. The reaction mixture was stirred at 25° C. for another 1 h, then concentrated in vacuum. The residue was purified by reverse phase flash chromatography (0.1% TFA) and then purified by prep-HPLC (column: Waters Xbridge C18, 150 mm×50 mm×10 m; mobile phase: [A: water (10 mM NH₄HCO₃), B: MeCN]; B %: 10%-40%, 11 min) to give the title compound (114.14 mg, 21.0% yield, 97.7% purity on LCMS) as a white solid.

LCMS: m/z 480.1 (M+H)⁺ (ES⁺).

¹H NMR (CD₃OD): δ 8.14 (d, 1H), 7.92 (d, 1H), 7.72 (s, 1H), 7.64 (d, 1H), 7.34 (d, 1H), 6.75 (d, 1H), 6.50 (s, 1H), 4.68-4.55 (m, 2H), 3.69 (s, 2H), 3.18-3.10 (m, 1H), 1.76 (s, 6H), 1.27 (d, 6H). One exchangeable proton not observed.

### Example 47: 4-(difluoromethoxy)-6-isopropyl-16,16-dimethyl-11,11-dioxo-18-oxa-11λ⁶-thia-10,14,15,20-tetrazatetracyclo[17-3-1-1^{12,15}.0^{2,7}]tetracosa-1(23),2(7),3,5,12(24), 13,19,21-octaen-9-one

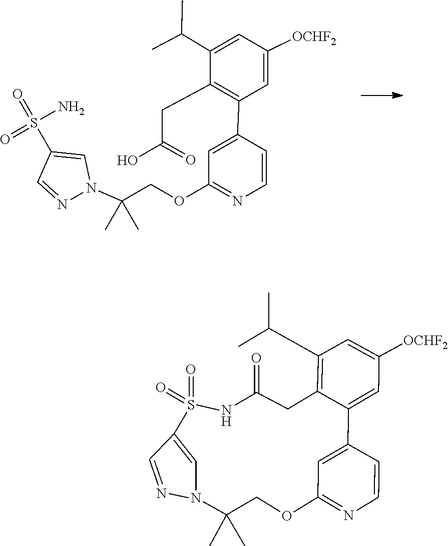

To a solution of 2-(4-(difluoromethoxy)-2-isopropyl-6-(2-(2-methyl-2-(4-sulfamoyl-1H-pyrazol-1-yl)propoxy)pyridin-4-yl)phenyl)acetic acid (Intermediate C47) (0.5 g, 928 μmol) in DCM (10 mL) was added dropwise TEA (376 mg, 3.71 mmol) and BOP (821 mg, 1.86 mmol) at 25° C. After addition, the mixture was stirred at this temperature for 1 h, and then DMAP (454 mg, 3.71 mmol) was added to the reaction mixture at 25° C. The resulting mixture was stirred at 25° C. for 20 min, then concentrated under reduced pressure. The residue was purified by reverse phase flash chromatography (water (0.1% TFA)-MeCN) and then purified by prep-HPLC (column: Waters Xbridge C18, 150 mm×25 mm×5 μm; mobile phase: [A: water (10 mM NH₄HCO), B: MeCN]; B %: 16%-46%, 10 min) to give the title compound (150 mg, 30.4% yield, 98% purity on HPLC) as a white solid.

LCMS: m/z 521.1 (M+H)⁺ (ES⁺).

¹H NMR (CDCl₃): δ 8.04 (d, 1H), 7.97 (d, 1H), 7.72 (s, 1H), 7.12 (d, 1H), 6.80 (d, 1H), 6.72 (s, 1H), 6.54 (t, 1H), 6.50 (s, 1H), 4.63 (s, 2H), 3.62 (s, 2H), 3.07-3.01 (m, 1H), 1.74 (s, 6H), 1.27 (d, 6H). One exchangeable proton not observed.

### Example 48: 5-fluoro-6-isopropyl-16,16-dimethyl-9,11,11-trioxo-18-oxa-11λ⁶-thia-10,14,15,20-tetrazatetracyclo[17-3-1-1^{12,15}.0^{2,7}]tetracosa-1(23),2(7),3,5,12(24),13,19,21-octaene-4-carbonitrile

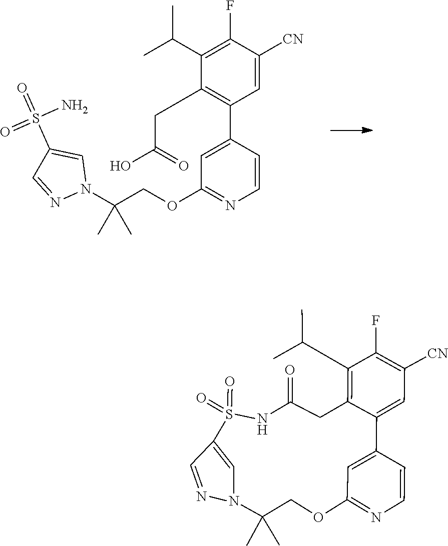 [SMILES: CC(C)c1c(F)c(C#N)cc(-c2ccnc(OCC(C)(C)n3cc(S(N)(=O)=O)cn3)c2)c1CC(=O)O ; CC(C)c1c(F)c(C#N)cc2c1CC(=O)NS(=O)(=O)c1cnn(c1)C(C)(C)COc1cc-2ccn1]

To a solution of 2-(4-cyano-3-fluoro-2-isopropyl-6-(2-(2-methyl-2-(4-sulfamoyl-1H-pyrazol-1-yl)propoxy)pyridin-4-yl)phenyl)acetic acid (Intermediate C48) (0.35 g, 634 μmol, HCl salt) in DCM (100 mL) was added TEA (257 mg, 2.54 mmol) and BOP (561 mg, 1.27 mmol). The solution was stirred at 25° C. for 1 h, then DMAP (232 mg, 1.90 mmol) was added and the solution was stirred at 25° C. for another 1 h. The solution was concentrated in vacuum. The residue was purified by reverse phase flash chromatography (water (0.1% TFA)-MeCN) and then prep-HPLC (column: Waters Xbridge C18, 150 mm×25 mm×5 μm; mobile phase: [A: water (10 mM NH₄HCO₃), B: MeCN]; B %: 17%-47%, 9 min) to give the title compound (122 mg, 38.3% yield, 99.5% purity on LCMS) as a white solid.

LCMS: m/z 498.1 (M+H)⁺ (ES⁺).

¹H NMR (DMSO-d₆): δ 11.85 (br s, 1H), 8.10 (s, 1H), 8.00 (d, 1H), 7.68 (s, 1H), 7.60 (d, 1H), 6.86 (s, 1H), 6.33 (s, 1H), 4.59 (s, 2H), 3.67 (s, 2H), 3.21-3.18 (m, 1H), 1.69 (s, 6H), 1.28 (d, 6H).

### Example 49: 4-(difluoromethoxy)-5-fluoro-6-isopropyl-16,16-dimethyl-11,11-dioxo-18-oxa-11λ⁶-thia-10,14,15,20-tetrazatetracyclo[17.3.1.1^{12,15}.0^{2,7}]tetracosa-1(23),2(7),3,5, 12(24),13,19,21-octaen-9-one

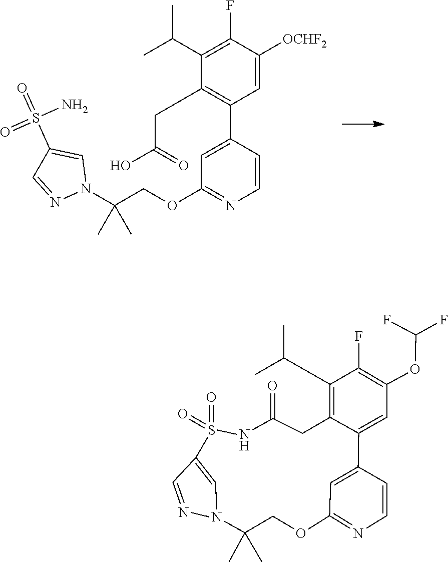 [SMILES: CC(C)c1c(F)c(OC(F)F)cc(-c2ccnc(OCC(C)(C)n3cc(S(N)(=O)=O)cn3)c2)c1CC(=O)O ; CC(C)c1c(F)c(OC(F)F)cc2c1CC(=O)NS(=O)(=O)c1cnn(c1)C(C)(C)COc1cc-2ccn1]

To a solution of 2-(4-(difluoromethoxy)-3-fluoro-2-isopropyl-6-(2-(2-methyl-2-(4-sulfamoyl-1H-pyrazol-1-yl)propoxy)pyridin-4-yl)phenyl)acetic acid (Intermediate C49) (0.4 g, 592 μmol, 99.3% purity, TFA salt) in DCM (100 mL) was added BOP (524 mg, 1.18 mmol) and TEA (240 mg, 2.37 mmol). The mixture was stirred at 25° C. for 1 h, then concentrated in vacuum. The residue was purified by reverse phase flash chromatography (0.1% TFA) and then purified by prep-HPLC (column: Waters Xbridge C18, 150 mm×50 mm×10 μm; mobile phase: [A: water (10 mM NH₄HCO₃), B: MeCN]; B %: 18%-48%, 10 min) to give the title compound (115 mg, 35.4% yield, 98.7% purity on LCMS) as a white solid.

LCMS: m/z 539.1 (M+H)⁺ (ES⁺).

¹H NMR (CD₃OD): δ 8.22 (s, 1H), 7.97 (d, 1H), 7.68 (s, 1H), 6.95 (d, 1H), 6.68 (m, 1H), 6.76 (d, 1H), 6.51 (s, 1H), 4.60 (s, 2H), 3.54 (s, 2H), 3.17-3.14 (m, 1H), 1.79 (s, 6H), 1.39 (d, 6H). One exchangeable proton not observed.

### Example 50: 4-fluoro-6-isopropyl-N,N,16,16-tetramethyl-9,11,11-trioxo-18-oxa-11λ⁶-thia-10,15,20,24-tetrazatetracyclo[17.3.1.1^{12,15}.0^{2,7}]tetracosa-1(23),2(7),3,5,12(24),13,19, 21-octaene-13-carboxamide

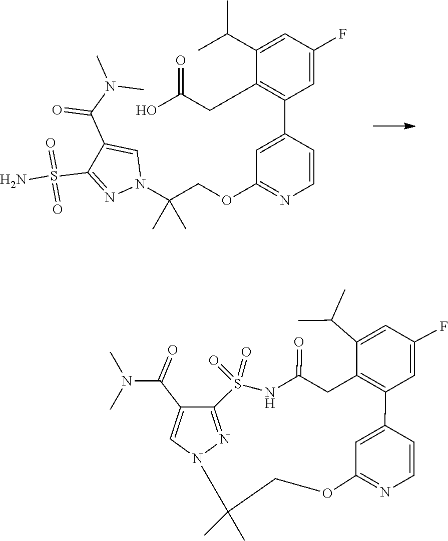 [SMILES: CC(C)c1cc(F)cc(-c2ccnc(OCC(C)(C)n3cc(C(=O)N(C)C)c(S(N)(=O)=O)n3)c2)c1CC(=O)O ; CC(C)c1cc(F)cc2c1CC(=O)NS(=O)(=O)c1nn(cc1C(=O)N(C)C)C(C)(C)COc1cc-2ccn1]

To a solution of 2-(2-(2-(2-(4-(dimethylcarbamoyl)-3-sulfamoyl-1H-pyrazol-1-yl)-2-methylpropoxy)pyridin-4-yl)-4-fluoro-6-isopropylphenyl)acetic acid (Intermediate C50) (150 mg, 222 μmol, TFA salt) in DCM (60 mL) was added TEA (84 mg, 833 μmol) and BOP (147 mg, 333 μmol), and then the mixture was stirred at 25° C. for 1 h. To the mixture was added DMAP (102 mg, 833 μmol) and the resulting mixture was stirred at 25° C. for 12 h. The reaction mixture was quenched with H₂O (10 mL) and then adjusted with 0.5 M aq HCl to pH 6. The layers were separated and the organic phase was washed with brine (30 mL), dried over anhydrous Na₂SO₄, filtered and concentrated in vacuum. The residue was purified by prep-HPLC (column: Phenomenex Gemini NX-C18, 75 mm×30 mm×3 μm; mobile phase: [A: water (10 mM NH₄HCO₃), B: MeCN]; B %: 14%-44%, 8 min) to give the title compound (65.1 mg, 53.9% yield, 100% purity on LCMS) as a white solid.

LCMS: m/z 544.4 (M+H)⁺ (ES⁺).

¹H NMR (CD₃OD): δ 8.17 (d, 1H), 8.02 (s, 1H), 7.09 (d, 1H), 6.86 (d, 1H), 6.75 (d, 1H), 6.33 (s, 1H), 4.65 (s, 2H), 3.42 (s, 2H), 3.05-2.97 (m, 7H), 1.71 (s, 6H), 1.23 (d, 6H). One exchangeable proton not observed.

### Example 51: 6-isopropyl-4-(methoxymethyl)-16,16-dimethyl-11,11-dioxo-18-oxa-11λ⁶-thia-10,14,15,20-tetrazatetracyclo[17-3-1-1^{12,15}.0^{2,7}]tetracosa-1(23),2(7),3,5,12(24),13,19, 21-octaen-9-one

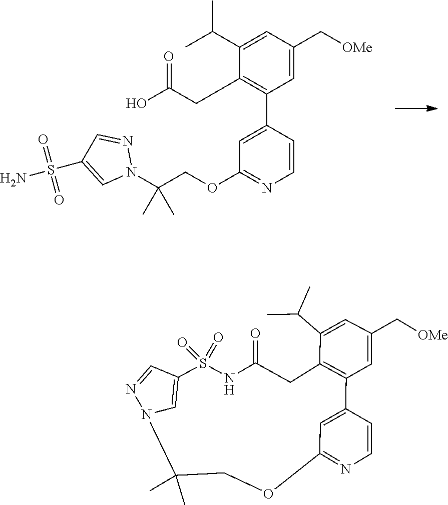

To a solution of 2-(2-isopropyl-4-(methoxymethyl)-6-(2-(2-methyl-2-(4-sulfamoyl-1H-pyrazol-1-yl)propoxy)pyridin-4-yl)phenyl)acetic acid (Intermediate C51) (0.4 g, 774 μmol) in DCM (50 mL) was added TEA (431.0 μL, 3.10 mmol) and BOP (685 mg, 1.55 mmol). The mixture was stirred at 25° C. for 1 h, then DMAP (378 mg, 3.10 mmol) was added. The resulting mixture was stirred at 25° C. for another 1 h, then concentrated in vacuum. The residue was purified by reverse phase flash chromatography (water (0.1% TFA)-MeCN) and then purified by prep-HPLC (column: Waters Xbridge C18, 150 mm×25 mm×5 μm; mobile phase: [A: water (10 mM NH₄HCO₃), B: MeCN]; B %: 7%-43%, 9 min) to give the title compound (133 mg, 34.0% yield, 99% purity on LCMS) as a white solid.

LCMS: m/z 499.4 (M+H)⁺ (ES⁺).

¹H NMR (CD₃OD): δ 8.16 (s, 1H), 7.89 (d, 1H), 7.66 (s, 1H), 7.35 (s, 1H), 6.94 (s, 1H), 6.73 (d, 1H), 6.50 (s, 1H), 4.61 (s, 2H), 4.45 (s, 2H), 3.60 (s, 2H), 3.38 (s, 3H), 3.11-3.04 (m, 1H), 1.77 (s, 6H), 1.25 (d, 6H). One exchangeable proton not observed.

### Example 52: 5-fluoro-6-isopropyl-4-(methoxymethyl)-16,16-dimethyl-11,11-dioxo-18-oxa-11λ⁶-thia-10,14,15,20-tetrazatetracyclo[17.3.1.1^{12,15}.0^{2,7}]tetracosa-1(23),2(7),3,5, 12(24),13,19,21-octaen-9-one

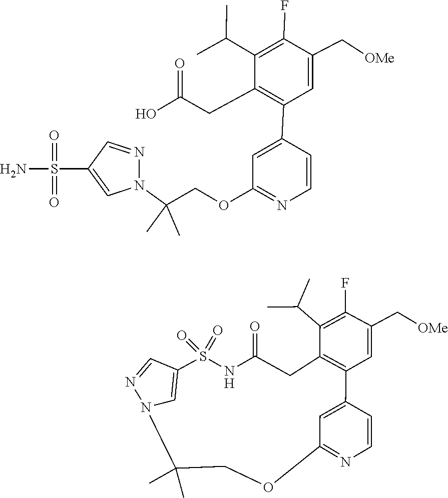

To a solution of 2-(3-fluoro-2-isopropyl-4-(methoxymethyl)-6-(2-(2-methyl-2-(4-sulfamoyl-1H-pyrazol-1-yl)propoxy)pyridin-4-yl)phenyl)acetic acid (Intermediate C52) (320 mg, 599 μmol) in DCM (5 mL) was added dropwise TEA (242 mg, 2.39 mmol) and BOP (529 mg, 1.20 mmol) at 25° C. The reaction mixture was stirred at 25° C. for 1 h, then DMAP (146 mg, 1.20 mmol) was added at 25° C. and the resulting mixture was stirred for 1 h. The reaction mixture was concentrated in vacuum. The residue was purified by reverse phase flash chromatography (water (0.1% TFA)-MeCN) then prep-HPLC (column: Phenomenex Gemini-NX C18, 75 mm×30 mm×3 μm; mobile phase: [A: water (10 mM NH₄HCO₃), B: MeCN]; B %: 12%-42%, 8 min) to give the title compound (98.9 mg, 31.9% yield) as a white solid.

LCMS: m/z 517.1 (M+H)⁺ (ES⁺).

¹H NMR (DMSO-d₆): δ 8.10 (s, 1H), 7.97 (d, 1H), 7.65 (s, 1H), 7.00 (d, 1H), 6.80-6.75 (m, 1H), 6.33 (s, 1H), 4.55 (s, 2H), 4.43 (s, 2H), 3.49 (s, 2H), 3.30 (s, 3H), 3.10-3.03 (m, 1H), 1.68 (s, 6H), 1.26 (d, 6H). One exchangeable proton not observed.

### Example 53: 4,13-difluoro-6-isopropyl-16,16-dimethyl-14-[(4-methylpiperazin-1-yl)-methyl]-11,11-dioxo-18-oxa-11λ⁶-thia-10,15,20,24-tetrazatetracyclo[17.3.1.1^{12,15}.0^{2,7}]-tetracosa-1(23),2(7),3,5,12(24),13,19,21-octaen-9-one

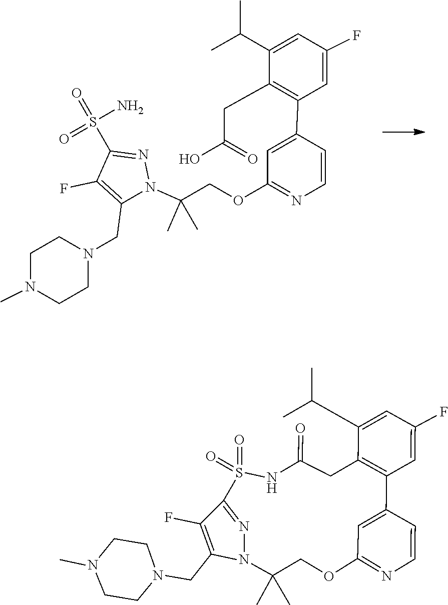 [SMILES: CC(C)c1cc(F)cc(-c2ccnc(OCC(C)(C)n3nc(S(N)(=O)=O)c(F)c3CN3CCN(C)CC3)c2)c1CC(=O)O ; CC(C)c1cc(F)cc2c1CC(=O)NS(=O)(=O)c1nn(c(CN3CCN(C)CC3)c1F)C(C)(C)COc1cc-2ccn1]

To a solution of 2-(4-fluoro-2-(2-(2-(4-fluoro-5-((4-methylpiperazin-1-yl)methyl)-3-sulfamoyl-1H-pyrazol-1-yl)-2-methylpropoxy)pyridin-4-yl)-6-isopropylphenyl)acetic acid (Intermediate C53) (120 mg, 163 μmol, TFA salt) in THF (15 mL) was added TEA (66 mg, 653 μmol) and BOP (144 mg, 327 μmol). The mixture was stirred at 20° C. for 1 h, then DMAP (80 mg, 653 μmol) was added. The mixture was stirred at 20° C. for 2 h, then concentrated in vacuum. The residue was purified by reverse phase flash chromatography (water (0.1% TFA)-MeCN) and then prep-HPLC (column: Phenomenex Gemini-NX C18, 75 mm×30 mm×3 μm; mobile phase: [A: water (10 mM NH₄HCO₃), B: MeCN]; B %: 18%-48%, 8 min) to give the title compound (9.12 mg, 9.26% yield, 99.9% purity on HPLC) as a white solid.

LCMS: m/z 603.4 (M+H)⁺ (ES⁺).

¹H NMR (CDCl₃): δ 12.22 (br s, 1H), 7.97 (s, 1H), 7.05 (dd, 1H), 6.77 (s, 1H), 6.69 (dd, 1H), 6.40 (s, 1H), 3.83-3.58 (m, 6H), 3.13-2.80 (m, 12H), 1.80 (s, 6H), 1.24 (d, 6H).

### Example 54: 4,13-difluoro-6-isopropyl-16,16-dimethyl-14-(4-methylpiperazine-1-carbonyl)-11,11-dioxo-18-oxa-11λ⁶-thia-10,15,20,24-tetrazatetracyclo[17.3.1.1^{12,15}.0^{2,7}]-tetracosa-1(23),2(7),3,5,12(24),13,19,21-octaen-9-one

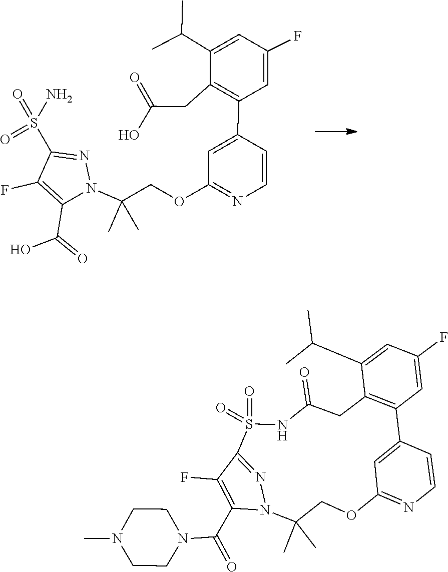 [SMILES: CC(C)c1cc(F)cc(-c2ccnc(OCC(C)(C)n3nc(S(N)(=O)=O)c(F)c3C(=O)O)c2)c1CC(=O)O ; CC(C)c1cc(F)cc2c1CC(=O)NS(=O)(=O)c1nn(c(C(=O)N3CCN(C)CC3)c1F)C(C)(C)COc1cc-2ccn1]

To a solution of 1-(1-((4-(2-(carboxymethyl)-5-fluoro-3-isopropylphenyl)pyridin-2-yl)-oxy)-2-methylpropan-2-yl)-4-fluoro-3-sulfamoyl-1H-pyrazole-5-carboxylic acid (Intermediate C54) (0.13 g, 235 μmol) in DCM (40 mL) was added TEA (95 mg, 941 μmol) and BOP (208 mg, 471 μmol). The mixture was stirred at 25° C. for 1 h, then DMAP (86 mg, 706 μmol) was added and the resulting mixture was stirred for another 1 h to achieve cyclisation. To the mixture was added TEA (45 mg, 449 μmol) and BOP (199 mg, 449 μmol) and the mixture was stirred at 25° C. for 10 min. Next 1-methyl-piperazine (45 mg, 449 μmol) was added and the resulting mixture was stirred at 25° C. for 20 min, then concentrated in vacuum. The residue was purified by reverse phase flash chromatography (water (0.1% TFA)-MeCN) and further purified by prep-HPLC (column: Waters Xbridge C18, 150 mm×25 mm×5 μm; mobile phase: [A: water (10 mM NH₄HCO₃), B: MeCN]; B %: 10%-43%, 9 min) to give the title compound (27.3 mg, 19.3% yield, 98.1% purity on LCMS) as a white a solid.

LCMS: m/z 617.3 (M+H)⁺ (ES⁺).

¹H NMR (CD₃OD): δ 8.19 (d, 1H), 7.09-7.06 (m, 1H), 6.88 (dd, 1H), 6.74-6.72 (m, 1H), 6.22 (s, 1H), 4.99-4.91 (m, 1H), 4.28-4.25 (m, 1H), 3.88-3.60 (m, 5H), 3.29-3.08 (m, 2H), 2.75-2.69 (m, 4H), 2.45 (s, 3H), 1.78-1.73 (m, 3H), 1.62-1.60 (m, 3H), 1.27-1.21 (m, 6H). One exchangeable proton not observed.

### Example 55: 4,13-difluoro-6-isopropyl-16,16-dimethyl-14-(morpholinomethyl)-11,11-dioxo-18-oxa-11λ⁶-thia-10,15,20,24-tetrazatetracyclo[17.3.1.1^{12,15}.0^{2,7}]tetracosa-1(23),2(7),3,5,12(24),13,19,21-octaen-9-one

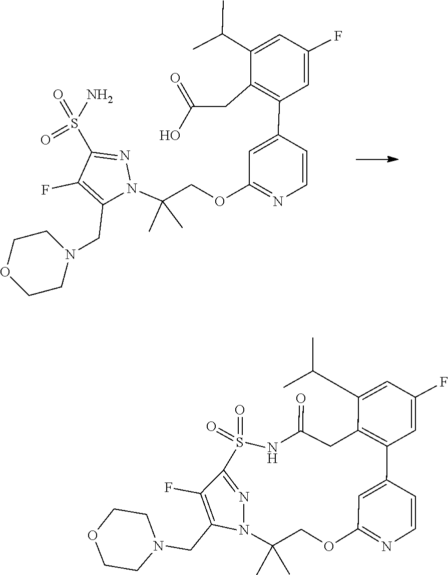 [SMILES: CC(C)c1cc(F)cc(-c2ccnc(OCC(C)(C)n3nc(S(N)(=O)=O)c(F)c3CN3CCOCC3)c2)c1CC(=O)O ; CC(C)c1cc(F)cc2c1CC(=O)NS(=O)(=O)c1nn(c(CN3CCOCC3)c1F)C(C)(C)COc1cc-2ccn1]

To a solution of 2-(4-fluoro-2-(2-(2-(4-fluoro-5-(morpholinomethyl)-3-sulfamoyl-1H-pyrazol-1-yl)-2-methylpropoxy)pyridin-4-yl)-6-isopropylphenyl)acetic acid (Intermediate C55) (0.1 g, 165 μmol) in THF (15 mL) was added TEA (67 mg, 658 μmol) and BOP (146 mg, 329 μmol). The reaction mixture was stirred at 20° C. for 0.5 h, then DMAP (80 mg, 658 μmol) was added. The resulting mixture was stirred at 20° C. for 12 h, then concentrated in vacuum. The residue was purified by prep-HPLC (column: Phenomenex Gemini-NX C18, 75 mm×30 mm×3 μm; mobile phase: [A: water (10 mM NH₄HCO₃); B: MeCN]; B %: 16%-46%, 8 min) to give the title compound (15.8 mg, 16.0% yield) as a white solid.

LCMS: m/z 590.3 (M+H)⁺ (ES⁺).

¹H NMR (CD₃OD): δ 8.14 (d, 1H), 7.09 (dd, 1H), 6.85 (d, 1H), 6.74 (dd, 1H), 6.24 (s, 1H), 4.81-4.75 (m, 2H), 3.69-3.66 (m, 4H), 3.62-3.59 (m, 2H), 3.50-3.45 (m, 2H), 3.10-3.00 (m, 1H), 2.50-2.41 (m, 4H), 1.81 (s, 6H), 1.24 (d, 6H). One exchangeable proton not observed.

### Example 56: 4-fluoro-6-isopropyl-16,16-dimethyl-11,11-dioxo-11λ⁶-thia-10,14,15,18, 20-pentazatetracyclo[17.3.1.1^{12,15}.0^{2,7}]tetracosa-1(23),2(7),3,5,12(24),13,19,21-octaen-9-one

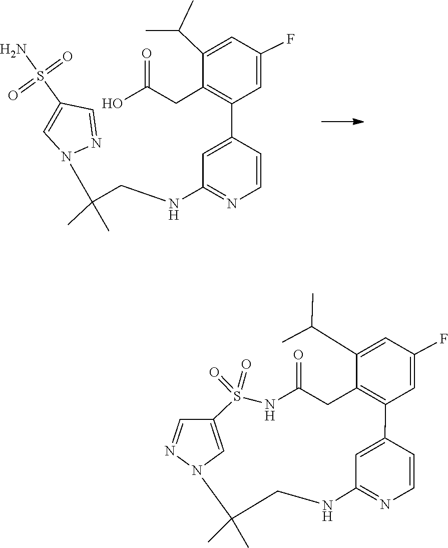 [SMILES: CC(C)c1cc(F)cc(-c2ccnc(NCC(C)(C)n3cc(S(N)(=O)=O)cn3)c2)c1CC(=O)O ; CC(C)c1cc(F)cc2c1CC(=O)NS(=O)(=O)c1cnn(c1)C(C)(C)CNc1cc-2ccn1]

To a solution of 2-(4-fluoro-2-isopropyl-6-(2-((2-methyl-2-(4-sulfamoyl-1H-pyrazol-1-yl)propyl)amino)pyridin-4-yl)phenyl)acetic acid (Intermediate C56) (0.08 g, 133 μmol, TFA salt) in DCM (40 mL) was added TEA (40 mg, 398 μmol) and BOP (117 mg, 265 μmol). The mixture was stirred at 25° C. for 1 h, then DMAP (49 mg, 398 μmol) was added and the resulting mixture was stirred for another 0.5 h. The mixture was concentrated in vacuum. The residue was purified by reverse phase flash chromatography (water (0.1% TFA)-MeCN) and then prep-HPLC (column: Phenomenex Gemini-NX C18, 75 mm×30 mm×3 μm; mobile phase: [A: water (10 mM NH₄HCO), B: MeCN]; B %: 10%-40%, 8 min) to give the title compound (19.62 mg, 31.3% yield, 99.7% purity on LCMS) as a white solid.

LCMS: m/z 472.2 (M+H)⁺ (ES⁺).

¹H NMR (CD₃CN): δ 7.96 (s, 1H), 7.80 (d, 1H), 7.65 (s, 1H), 7.09 (dd, 1H), 6.68 (dd, 1H), 6.31 (dd, 1H), 5.74 (br s, 1H), 5.37 (br s, 1H), 3.67-3.58 (m, 4H), 3.15-3.09 (m, 1H), 1.65 (s, 6H), 1.21 (d, 6H). One exchangeable proton not observed.

### Examples—Biological Studies

### NLRP3 and Pyroptosis

It is well established that the activation of NLRP3 leads to cell pyroptosis and this feature plays an important part in the manifestation of clinical disease (Yan-gang Liu et al., Cell Death & Disease, 2017, 8(2), e2579; Alexander Wree et al., Hepatology, 2014, 59(3), 898-910; Alex Baldwin et al., Journal of Medicinal Chemistry, 2016, 59(5), 1691-1710; Ema Ozaki et al., Journal of Inflammation Research, 2015, 8, 15-27; Zhen Xie & Gang Zhao, Neuroimmunology Neuroinflammation, 2014, 1(2), 60-65; Mattia Cocco et al., Journal of Medicinal Chemistry, 2014, 57(24), 10366-10382; T. Satoh et al., Cell Death & Disease, 2013, 4, e644). Therefore, it is anticipated that inhibitors of NLRP3 will block pyroptosis, as well as the release of pro-inflammatory cytokines (e.g. IL-1β) from the cell.

### THP-1 Cells: Culture and Preparation

THP-1 cells (ATCC #TIB-202) were grown in RPMI containing L-glutamine (Gibco #11835) supplemented with 1 mM sodium pyruvate (Sigma #S8636) and penicillin (100 units/ml)/streptomycin (0.1 mg/ml) (Sigma #P4333) in 10% Fetal Bovine Serum (FBS) (Sigma #F0804). The cells were routinely passaged and grown to confluency (˜10⁶ cells/ml). On the day of the experiment, THP-1 cells were harvested and resuspended into RPMI medium (without FBS). The cells were then counted and viability (>90%) checked by Trypan blue (Sigma #T8154). Appropriate dilutions were made to give a concentration of 625,000 cells/ml. To this diluted cell solution was added LPS (Sigma #L4524) to give a 1 g/ml Final Assay Concentration (FAC). 40 μl of the final preparation was aliquoted into each well of a 96-well plate. The plate thus prepared was used for compound screening.

### THP-1 Cells Pyroptosis Assay

The following method step-by-step assay was followed for compound screening.
1. Seed THP-1 cells (25,000 cells/well) containing 1.0 μg/ml LPS in 40 μl of RPMI medium (without FBS) in 96-well, black walled, clear bottom cell culture plates coated with poly-D-lysine (VWR #734-0317)
2. Add 5 μl compound (8 points half-log dilution, with 10 μM top dose) or vehicle (DMSO 0.1% FAC) to the appropriate wells
3. Incubate for 3 hrs at 37° C., 5% CO₂
4. Add 5 μl nigericin (Sigma #N7143) (FAC 5 μM) to all wells
5. Incubate for 1 hr at 37° C., 5% CO₂
6. At the end of the incubation period, spin plates at 300×g for 3 mins and remove supernatant
7. Then add 50 μl of resazurin (Sigma #R7017) (FAC 100 μM resazurin in RPMI medium without FBS) and incubate plates for a further 1-2 hrs at 37° C. and 5% CO₂
8. Plates were read in an Envision reader at Ex 560 nm and Em 590 nm
9. IC₅₀ data is fitted to a non-linear regression equation (log inhibitor vs response-variable slope 4-parameters)

### 96-Well Plate Map

| | 1 | 2 | 3 | 4 | 5 | 6 | 7 | 8 | 9 | 10 | 11 | 12 |
|---|---|---|---|---|---|---|---|---|---|---|---|---|
| A | High | Comp 1 | Comp 2 | Comp 3 | Comp 4 | Comp 5 | Comp 6 | Comp 7 | Comp 8 | Comp 9 | Comp 10 | Low |
| B | High | Comp 1 | Comp 2 | Comp 3 | Comp 4 | Comp 5 | Comp 6 | Comp 7 | Comp 8 | Comp 9 | Comp 10 | Low |
| C | High | Comp 1 | Comp 2 | Comp 3 | Comp 4 | Comp 5 | Comp 6 | Comp 7 | Comp 8 | Comp 9 | Comp 10 | Low |
| D | High | Comp 1 | Comp 2 | Comp 3 | Comp 4 | Comp 5 | Comp 6 | Comp 7 | Comp 8 | Comp 9 | Comp 10 | Low |
| E | High | Comp 1 | Comp 2 | Comp 3 | Comp 4 | Comp 5 | Comp 6 | Comp 7 | Comp 8 | Comp 9 | Comp 10 | Low |
| F | High | Comp 1 | Comp 2 | Comp 3 | Comp 4 | Comp 5 | Comp 6 | Comp 7 | Comp 8 | Comp 9 | Comp 10 | Low |
| G | High | Comp 1 | Comp 2 | Comp 3 | Comp 4 | Comp 5 | Comp 6 | Comp 7 | Comp 8 | Comp 9 | Comp 10 | Low |
| H | High | Comp 1 | Comp 2 | Comp 3 | Comp 4 | Comp 5 | Comp 6 | Comp 7 | Comp 8 | Comp 9 | Comp 10 | Low |

High MCC950 (10 uM)
Low Drug free control
Compound 8-point half-log dilution

The results of the pyroptosis assay are summarised in Table 1 below as THP IC₅₀.

### Human Whole Blood IL-1β Release Assay

For systemic delivery, the ability to inhibit NLRP3 when the compounds are present within the bloodstream is of great importance. For this reason, the NLRP3 inhibitory activity of a number of compounds in human whole blood was investigated in accordance with the following protocol.

Human whole blood in Li-heparin tubes was obtained from healthy donors from a volunteer donor panel.
1. Plate out 80 μl of whole blood containing 1 μg/ml of LPS in 96-well, clear bottom cell culture plate (Corning #3585)
2. Add 10 μl compound (8 points half-log dilution with 10 μM top dose) or vehicle (DMSO 0.1% FAC) to the appropriate wells
3. Incubate for 3 hrs at 37° C., 5% CO₂
4. Add 10 μl nigericin (Sigma #N7143) (10 μM FAC) to all wells
5. Incubate for 1 hr at 37° C., 5% CO₂
6. At the end of the incubation period, spin plates at 300×g for 5 mins to pellet cells and remove 201 of supernatant and add to 96-well v-bottom plates for IL-1β analysis (note: these plates containing the supernatants can be stored at −80° C. to be analysed at a later date)
7. IL-1β was measured according to the manufacturer protocol (Perkin Elmer-AlphaLisa IL-1 Kit AL220F-5000)
8. IC₅₀ data is fitted to a non-linear regression equation (log inhibitor vs response-variable slope 4-parameters)

The results of the human whole blood assay are summarised in Table 1 below as HWB IC₅₀.

TABLE 1
NLRP₃ inhibitory activity
(≤0.1 μM = ‘+++++’, ≤0.5 μM = ‘++++’,
≤1 μM = ‘+++’, ≤5 μM = ‘++’, ≤10 μM = ‘+’,
not determined = ‘ND’).

| Example | THP | HWB |
|---|---|---|
| No | IC₅₀ | IC₅₀ |
| 1 | ++ | ++ |
| 2 | ++ | +++ |
| 3 | + | + |
| 4 | ++ | + |
| 5 | ++++ | ++++ |
| 6 | + | ND |
| 7 | ++ | ND |
| 8 | ++ | ND |
| 9 | + | ND |
| 10 | ++ | ++ |
| 11 | ++++ | ++ |
| 12 | +++ | +++ |
| 13 | ++ | ++ |
| 14 | ++ | ND |
| 15 | ++++ | ++ |
| 16 | ++++ | ++++ |
| 17 | +++ | ++ |
| 18 | ++ | ND |
| 19 | ++++ | +++ |
| 20 | +++ | ++ |
| 21 | ++ | + |
| 22 | ++ | + |
| 23 | + | ND |
| 24 | +++ | ++ |
| 25 | +++++ | ++++ |
| 26 | ++ | ND |
| 27 | ++ | +++ |
| 28 | ++ | ND |
| 29 | ++++ | ++++ |
| 30 | +++ | ND |
| 31 | ++ | ++ |
| 32 | +++ | ++++ |
| 33 | +++ | ++ |
| 34 | ++++ | ++ |
| 35 | +++++ | +++ |
| 36 | ++ | ++ |
| 37 | +++ | ++ |
| 38 | ++ | ++ |
| 39 | ++ | ++ |
| 40 | ++ | ++ |
| 41 | ++ | ++ |
| 42 | +++++ | ++++ |
| 43 | ++ | ND |
| 44 | ++ | + |
| 45 | ++ | ND |
| 46 | ++ | ND |
| 47 | ++++ | ND |
| 48 | ++ | ND |
| 49 | +++ | ND |
| 50 | + | ND |
| 51 | +++++ | ND |
| 52 | ++++ | ND |
| 53 | +++++ | ND |
| 54 | +++ | ND |
| 55 | ++++ | ND |
| 56 | +++++ | ND |

As is evident from the results presented in Table 1, surprisingly in spite of the structural differences versus the prior art compounds, the compounds of the invention show high levels of NLRP3 inhibitory activity in the pyroptosis assay and in the human whole blood assay.

It will be understood that the present invention has been described above by way of example only. The examples are not intended to limit the scope of the invention. Various modifications and embodiments can be made without departing from the scope and spirit of the invention, which is defined by the following claims only.

1. A compound of formula (I): 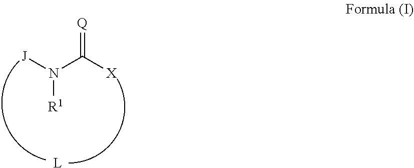
or a pharmaceutically acceptable salt or solvate thereof, wherein:
J is —SO—, —SO₂— or —SO(═NR^{j})—;
Q is O or S;
X is —C(R²)₂—;
L is a saturated or unsaturated hydrocarbylene group, wherein the hydrocarbylene group may be straight-chained or branched, or be or include one or more cyclic groups, wherein the hydrocarbylene group may optionally be substituted, and wherein the hydrocarbylene group may optionally include one or more heteroatoms independently selected from N, O and S in its carbon skeleton;
-J-N(R¹)—C(=Q)-X— and -L- together form a ring, such that the minimum single ring size that encompasses all or part of each of -J-, —N(R¹)—, —C(=Q)-, —X— and -L- is from 8 to 30 atoms;
each R^{j} and R¹ is independently selected from hydrogen or a saturated or unsaturated hydrocarbyl group, wherein the hydrocarbyl group may be straight-chained or branched, or be or include one or more cyclic groups, wherein the hydrocarbyl group may optionally be substituted, and wherein the hydrocarbyl group may optionally include one or more heteroatoms independently selected from N, O and S in its carbon skeleton; and
each R² is independently selected from hydrogen or a halo, —OH, —NO₂, —NH₂, —N₃, —SH, —SO₂H, —SO₂NH₂, or a saturated or unsaturated hydrocarbyl group, wherein the hydrocarbyl group may be straight-chained or branched, or be or include one or more cyclic groups, wherein the hydrocarbyl group may optionally be substituted, and wherein the hydrocarbyl group may optionally include one or more heteroatoms N, O or S in its carbon skeleton, or wherein two R² may, together with the carbon atom to which they are attached, form a cyclic group, wherein the cyclic group may optionally be substituted.

2. A compound or a pharmaceutically acceptable salt or solvate thereof, as claimed in claim 1, wherein:
(i) J is —SO₂—; and/or
(ii) Q is O; and/or
(iii) R¹ is hydrogen and X is —CH₂—; and/or
(iv) L is a saturated or unsaturated hydrocarbylene group, wherein the hydrocarbylene group may be straight-chained or branched, wherein the hydrocarbylene group includes an aromatic cyclic group directly attached to X, wherein the hydrocarbylene group may optionally include one or more further cyclic groups, wherein the hydrocarbylene group may optionally be substituted, and wherein the hydrocarbylene group may optionally include one or more heteroatoms independently selected from N, O and S in its carbon skeleton; and or
(v) the minimum single ring size that encompasses all or part of each of -J-, —N(R¹)—, —C(=Q)-, —X— and -L- is from 12 to 24 atoms, or from 14 to 20 atoms.

3. (canceled)

4. (canceled)

5. (canceled)

6. A compound or a pharmaceutically acceptable salt or solvate thereof, as claimed in claim 1, wherein the compound has the formula (Ia): 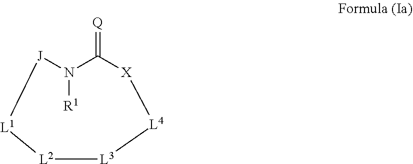
wherein:
J, R¹, Q and X are as previously defined;
-J-N(R¹)—C(=Q)-X- and -L¹-L²-L³-L⁴- together form a ring, such that the minimum single ring size that encompasses all or part of each of -J-, —N(R¹)—, —C(=Q)-, —X—, -L¹-, -L²-, -L³- and -L⁴- is from 8 to 30 atoms;
L¹ is a bond, a divalent 3- to 7-membered monocyclic group, a divalent 5- to 12-membered bicyclic group, or a divalent 7- to 18-membered tricyclic group, any of which may optionally be substituted with one or more monovalent substituents and/or π-bonded substituents;
L² is an alkylene, alkenylene or alkynylene group, wherein the alkylene, alkenylene or alkynylene group may be straight-chained or branched, or be or include one or more cyclic groups, wherein one or more carbon atoms in the backbone of the alkylene, alkenylene or alkynylene group may optionally be replaced by one or more heteroatoms independently selected from N, O and S, and wherein the alkylene, alkenylene or alkynylene group may optionally be substituted with one or more monovalent substituents, and/or one or more π-bonded substituents;
L³ is a bond, a divalent 3- to 7-membered monocyclic group, a divalent 5- to 12-membered bicyclic group, or a divalent 7- to 18-membered tricyclic group, any of which may optionally be substituted with one or more monovalent substituents and/or π-bonded substituents; and
L⁴ is a divalent 3- to 7-membered monocyclic group, a divalent 5- to 12-membered bicyclic group, or a divalent 7- to 18-membered tricyclic group, any of which may optionally be substituted with one or more monovalent substituents and/or π-bonded substituents, and optionally wherein the ring of the divalent monocyclic, bicyclic or tricyclic group of L⁴ that is directly attached to X is aromatic.

7. A compound or a pharmaceutically acceptable salt or solvate thereof, as claimed in claim 6, wherein:
(i) L¹ is a divalent 3- to 7-membered monocyclic group, or a divalent 7- to 11-membered bicyclic group, either of which may optionally be substituted with one or more monovalent substituents and/or π-bonded substituents; and/or
(ii) L³ is a divalent phenyl or 5- or 6-membered monocyclic heteroaryl group, any of which may optionally be substituted with one or more monovalent substituents; and/or
(iii) the minimum single ring size that encompasses all or part of each of -J-, —N(R¹)—, —C(=Q)-, —X—, -L¹-, -L²-, -L³- and -L⁴- is from 12 to 24 atoms, or from 14 to 20 atoms.

8. A compound or a pharmaceutically acceptable salt or solvate thereof, as claimed in claim 6, wherein:
(i) L² is an alkylene or alkenylene group, wherein the alkylene or alkenylene group may be straight-chained or branched, or include a single monocyclic group, wherein one or more carbon atoms in the backbone of the alkylene or alkenylene group may optionally be replaced by one or more heteroatoms independently selected from N, O and S, and wherein the alkylene or alkenylene group may optionally be substituted with one or more monovalent substituents, and/or one or more π-bonded substituents; or
(ii) L² is an alkylene or alkenylene group, wherein the alkylene or alkenylene group is straight-chained or branched, wherein one or more carbon atoms in the backbone of the alkylene or alkenylene group may optionally be replaced by one or more heteroatoms independently selected from N, O and S, and wherein the alkylene or alkenylene group may optionally be substituted with one or more monovalent substituents, and/or one or more π-bonded substituents.

9. (canceled)

10. (canceled)

11. (canceled)

12. A compound or a pharmaceutically acceptable salt or solvate thereof, as claimed in claim 1, wherein the compound has the formula (Ib): 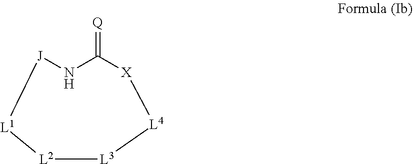
wherein:
J is —SO—, —SO₂— or —SO(═NH)—;
X is —CH₂—;
-J-NH—C(═O)—X- and -L¹-L²-L³-L⁴- together form a ring, such that the minimum single ring size that encompasses all or part of each of -J-NH—C(═O)—X—, -L¹-, -L²-, -L³- and -L⁴- is from 8 to 30 atoms;
L¹ is a bond, a divalent 3- to 7-membered monocyclic group, or a divalent 7- to 11-membered bicyclic group, wherein the divalent 3- to 7-membered monocyclic group or divalent 7- to 11-membered bicyclic group may optionally be substituted with one or more halo groups and/or one or more oxo (═O) groups and/or one or more substituents R^{L};
L² is an alkylene or alkenylene group, wherein the alkylene or alkenylene group may be straight-chained or branched, or be or include one or more cyclic groups, wherein one or more carbon atoms in the backbone of the alkylene or alkenylene group may optionally be replaced by one or more heteroatoms independently selected from N and O, and wherein the alkylene or alkenylene group may optionally be substituted with one or more halo groups;
L³ is a divalent phenyl or 5- or 6-membered heteroaryl group, wherein the divalent phenyl or 5- or 6-membered heteroaryl group may optionally be substituted with one or more halo groups and/or one or more substituents R^{L};
L⁴ is a divalent phenyl or 5- or 6-membered heteroaryl group, wherein the divalent phenyl or 5- or 6-membered heteroaryl group may optionally be substituted with one or more halo groups and/or one or more substituents R^{L};
the ring atom of L⁴ that is directly attached to L³ is at the α-position relative to the ring atom of L⁴ that is directly attached to X;
each R^{L} is independently selected from a C₁-C₆ alkyl, C₁-C₆ haloalkyl, C₂-C₆ alkenyl, C₂-C₆ haloalkenyl, —R¹¹-R¹², —R¹¹—CN, —R¹¹—N₃, —R¹¹—NO₂, —R¹¹—N(R¹³)₂, —R¹¹—OR¹³, —R¹¹—COR¹³, —R¹¹—COOR¹³, —R¹¹—CON(R¹³)₂, —R¹¹—C(═NR¹³)R¹³, —R¹¹—C(═NR¹³)N(R¹³)₂, —R¹¹—C(═NOR¹³)R¹³, —R¹¹—SO₂R¹³ or —R¹¹—SO₂N(R¹³)₂ group, and/or any two R^{L} attached to the same divalent phenyl or 5- or 6-membered heteroaryl group of L³ or L⁴ may, together with the atoms of the divalent phenyl or 5- or 6-membered heteroaryl group to which they are attached, form a fused 5- or 6-membered cyclic group, wherein the fused 5- or 6-membered cyclic group may optionally be substituted with one or more halo groups and/or one or two oxo (═O) groups and/or one, two or three substituents independently selected from a C₁-C₆ alkyl, C₁-C₆ haloalkyl, C₂-C₆ alkenyl, C₂-C₆ haloalkenyl, —R¹¹-R¹², —R¹¹—CN, —R¹¹—N₃, —R¹¹—NO₂, —R¹¹—N(R¹³)₂, —R¹¹—OR¹³, —R¹¹—COR¹³, —R¹¹—COOR¹³, —R¹¹—CON(R¹³)₂, —R¹¹—C(═NR¹³)R¹³, —R¹¹—C(═NR¹³)N(R¹³)₂, —R¹¹—C(═NOR¹³)R¹³, —R¹¹—SO₂R¹³ or —R¹¹—SO₂N(R¹³)₂ group;
each R¹¹ is independently selected from a bond or a C₁-C₄ alkylene group, wherein the C₁-C₄ alkylene group may be straight-chained or branched, or be or include a C₃-C₄ cycloalkylene group, and wherein the C₁-C₄ alkylene group may optionally be substituted with one or more halo groups;
each R¹² is independently selected from a 3- to 6-membered cyclic group, wherein the 3- to 6-membered cyclic group may optionally be substituted with one or more halo groups and/or one, two or three substituents independently selected from —CN, —NO₂, —R¹⁴, —OH, —OR¹⁴, —NH₂, —NHR¹⁴ and —N(R¹⁴)₂;
each R¹³ is independently selected from hydrogen or a C₁-C₆ alkyl, C₁-C₆ haloalkyl, C₂-C₆ alkenyl, C₂-C₆ haloalkenyl, or 3- to 6-membered cyclic group, wherein the 3- to 6-membered cyclic group may optionally be substituted with one or more halo groups and/or one, two or three substituents independently selected from —CN, —NO₂, —R¹⁴, —OH, —OR¹⁴, —NH₂, —NHR¹⁴ and —N(R¹⁴)₂, or any two R¹³ attached to the same nitrogen atom may together form a C₂-C₅ alkylene or C₂-C₅ haloalkylene group; and
each R¹⁴ is independently selected from a C₁-C₄ alkyl or C₁-C₄ haloalkyl group.

13. A compound or a pharmaceutically acceptable salt or solvate thereof, as claimed in claim 12, wherein:
(i) L¹ is a divalent 3- to 7-membered monocyclic group, or a divalent 7- to 11-membered bicyclic group, wherein the divalent 3- to 7-membered monocyclic group or divalent 7- to 11-membered bicyclic group may optionally be substituted with one or more halo groups and/or one or more oxo (═O) groups and/or one or more substituents R^{L}; and/or
(ii) L² is an alkylene or alkenylene group, wherein the alkylene or alkenylene group may be straight-chained or branched, or include a single monocyclic group, wherein one or more carbon atoms in the backbone of the alkylene or alkenylene group may optionally be replaced by one or more heteroatoms independently selected from N and O, wherein the alkylene or alkenylene group may optionally be substituted with one or more halo groups and wherein L² contains in total from 2 to 15 carbon, nitrogen and oxygen atoms; and/or
(iii) the minimum single ring size that encompasses all or part of each of -J-NH—C(═O)—X—, -L¹-, -L²-, -L³- and -L⁴- is from 12 to 24 atoms, or from 14 to 20 atoms.

14. (canceled)

15. A compound or a pharmaceutically acceptable salt or solvate thereof, as claimed in claim 12, wherein:
(i) the divalent phenyl or 5- or 6-membered heteroaryl group of L⁴ is substituted at the α′-position, relative to the ring atom of L⁴ that is directly attached to X, with a C₁-C₆ alkyl, C₁-C₆ haloalkyl, C₂-C₆ alkenyl, C₂-C₆ haloalkenyl, or 3- to 6-membered cyclic group, wherein the 3- to 6-membered cyclic group may optionally be substituted with one or more halo groups; or
(ii) the divalent phenyl or 5- or 6-membered heteroaryl group of L⁴ is ortho-fused to a 5- or 6-membered cyclic group across the α′, β′-positions, relative to the ring atom of L⁴ that is directly attached to X, wherein the ortho-fused 5- or 6-membered cyclic group is optionally substituted with one or more halo groups.

16. (canceled)

17. (canceled)

18. A compound or a pharmaceutically acceptable salt or solvate thereof, as claimed in claim 1, wherein the compound has the formula (Ic): 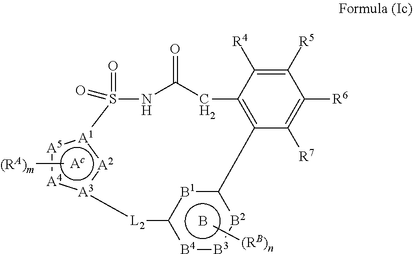
wherein:
A¹ and A³ are each independently selected from C and N, and A², A⁴ and A⁵ are each independently selected from N, C—H, C-Hal and N—H, such that ring A^{c} is a 5-membered heteroaryl ring containing one, two or three nitrogen atoms in its ring structure;
B¹, B², B³ and B⁴ are each independently selected from N, C—H and C-Hal, such that ring B is a 6-membered aryl ring or a 6-membered heteroaryl ring containing one, two or three nitrogen atoms in its ring structure;
m is 0, 1 or 2;
n is 0, 1 or 2;
each R^{A} is independently selected from —OH, —NH₂, —CN or a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{A} contains, in total, from 1 to 10 carbon, nitrogen and oxygen atoms, or wherein any two R^{A} attached to A⁴ and A⁵ may together form a fused 5- or 6-membered cyclic group, wherein the fused 5- or 6-membered cyclic group may optionally be substituted with one or more Hal groups and/or one or two groups independently selected from oxo (═O) and R^{AA};
each R^{AA} is independently selected from —OH, —NH₂, —CN or a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{AA} contains, in total, from 1 to 10 carbon, nitrogen and oxygen atoms;
each R^{B} is independently selected from a —CN, —NO₂, —R^{B1}, —OH, —OR^{B1}, —NH₂, —NHR^{B1} or —N(R^{B1})₂ group, wherein each R^{B1} is independently selected from a C₁-C₄ alkyl or C₁-C₄ fluoroalkyl group;
each Hal is independently selected from F, Cl or Br;
L² is a straight-chained alkylene or alkenylene group, wherein the straight-chained alkylene or alkenylene group optionally includes one or two heteroatoms independently selected from O and N in its carbon skeleton, wherein L² has a chain length of from 2 to 8 atoms, and wherein L² may optionally be substituted with one or two oxo (═O) groups and/or with one or more groups R^{L2}, wherein each R^{L2} is independently selected from a fluoro, C₁-C₄ alkyl, —O—(C₁-C₄ alkyl), C₁-C₄ fluoroalkyl or —O—(C₁-C₄ fluoroalkyl) group, or wherein any two R^{L2} may together with the atom(s) of the alkylene or alkenylene group to which they are attached form a 3- to 7-membered cyclic group, wherein the 3- to 7-membered cyclic group may optionally be substituted with one or more Hal groups and/or one or two oxo (═O) groups;
R⁴ is selected from a C₁-C₄ alkyl, C₁-C₄ fluoroalkyl, C₃-C₆ cycloalkyl or C₃-C₆ fluorocycloalkyl group, and R⁵ is selected from hydrogen, F, Cl, Br, or a —CN, methyl, fluoromethyl, —OC(R²⁰)₃ or —C(R²⁰)₂—OC(R²⁰)₃ group, or R⁴ and R⁵ together form a divalent group selected from —CH₂CH₂CH₂—, —CH═CHCH₂—, —CH₂CH═CH—, —CH₂CH₂O— and —OCH₂CH₂—, wherein the divalent group formed by R⁴ and R⁵ may optionally be fluoro-substituted;
R⁶ and R⁷ are each independently selected from hydrogen, F, Cl, Br, or a —CN, methyl, fluoromethyl, —OC(R²⁰)₃ or —C(R²⁰)₂—OC(R²⁰)₃ group; and
each R²⁰ is independently selected from hydrogen or F.

19. A compound or a pharmaceutically acceptable salt or solvate thereof, as claimed in claim 1, wherein the compound has the formula (Id): 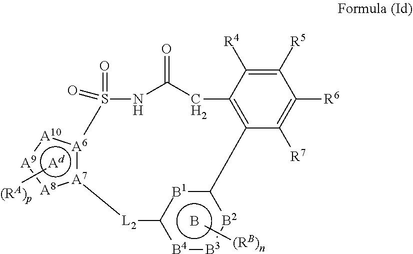
wherein:
A⁶ and A⁷ are each independently selected from C and N, and A⁸, A⁹ and A¹⁰ are each independently selected from N, C—H, C-Hal and N—H, such that ring A^{d} is a 5-membered heteroaryl ring containing one, two or three nitrogen atoms in its ring structure;
B¹, B², B³ and B⁴ are each independently selected from N, C—H and C-Hal, such that ring B is a 6-membered aryl ring or a 6-membered heteroaryl ring containing one, two or three nitrogen atoms in its ring structure;
p is 0, 1 or 2;
n is 0, 1 or 2;
each R^{A} is independently selected from —OH, —NH₂, —CN or a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{A} contains, in total, from 1 to 10 carbon, nitrogen and oxygen atoms, or wherein any two R^{A} attached to A⁸ and A⁹ or to A⁹ and A¹⁰ may together form a fused 5- or 6-membered cyclic group, wherein the fused 5- or 6-membered cyclic group may optionally be substituted with one or more Hal groups and/or one or two groups independently selected from oxo (═O) and R^{AA};
each R^{AA} is independently selected from —OH, —NH₂, —CN or a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{AA} contains, in total, from 1 to 10 carbon, nitrogen and oxygen atoms;
each R^{B} is independently selected from a —CN, —NO₂, —R^{B1}, —OH, —OR^{B1}, —NH₂, —NHR^{B1} or —N(R^{B1})₂ group, wherein each R^{B1} is independently selected from a C₁-C₄ alkyl or C₁-C₄ fluoroalkyl group;
each Hal is independently selected from F, Cl or Br;
L² is a straight-chained alkylene or alkenylene group, wherein the straight-chained alkylene or alkenylene group optionally includes one or two heteroatoms independently selected from O and N in its carbon skeleton, wherein L² has a chain length of from 2 to 8 atoms, and wherein L² may optionally be substituted with one or two oxo (═O) groups and/or with one or more groups R^{L2}, wherein each R^{L2} is independently selected from a fluoro, C₁-C₄ alkyl, —O—(C₁-C₄ alkyl), C₁-C₄ fluoroalkyl or —O—(C₁-C₄ fluoroalkyl) group, or wherein any two R^{L2} may together with the atom(s) of the alkylene or alkenylene group to which they are attached form a 3- to 7-membered cyclic group, wherein the 3- to 7-membered cyclic group may optionally be substituted with one or more Hal groups and/or one or two oxo (═O) groups;
R⁴ is selected from a C₁-C₄ alkyl, C₁-C₄ fluoroalkyl, C₃-C₆ cycloalkyl or C₃-C₆ fluorocycloalkyl group, and R⁵ is selected from hydrogen, F, Cl, Br, or a —CN, methyl, fluoromethyl, —OC(R²⁰)₃ or —C(R²⁰)₂—OC(R²⁰)₃ group or R⁴ and R⁵ together form a divalent group selected from —CH₂CH₂CH₂—, —CH═CHCH₂—, —CH₂CH═CH—, —CH₂CH₂O— and —OCH₂CH₂—, wherein the divalent group formed by R⁴ and R⁵ may optionally be fluoro-substituted;
R⁶ and R⁷ are each independently selected from hydrogen, F, Cl, Br, or a —CN, methyl, fluoromethyl, —OC(R²⁰)₃ or —C(R²⁰)₂—OC(R²⁰)₃ group; and
each R²⁰ is independently selected from hydrogen or F.

20. A compound or a pharmaceutically acceptable salt or solvate thereof, as claimed in claim 1, wherein the compound has the formula (Ie): 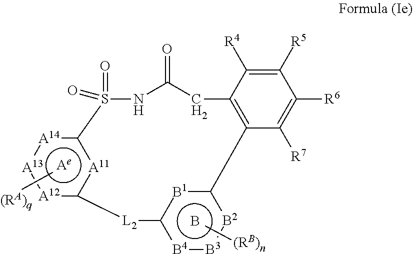
wherein:
A¹¹, A¹², A¹³ and A¹⁴ are each independently selected from N, C—H and C-Hal, such that ring Ae is a 6-membered aryl ring or a 6-membered heteroaryl ring containing one, two or three nitrogen atoms in its ring structure;
B¹, B², B³ and B⁴ are each independently selected from N, C—H and C-Hal, such that ring B is a 6-membered aryl ring or a 6-membered heteroaryl ring containing one, two or three nitrogen atoms in its ring structure;
q is 0, 1 or 2;
n is 0, 1 or 2;
each R^{A} is independently selected from —OH, —NH₂, —CN or a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{A} contains, in total, from 1 to 10 carbon, nitrogen and oxygen atoms, or wherein any two R^{A} attached to A¹² and A¹³ or to A¹³ and A¹⁴ may together form a fused 5- or 6-membered cyclic group, wherein the fused 5- or 6-membered cyclic group may optionally be substituted with one or more Hal groups and/or one or two groups independently selected from oxo (═O) and R^{AA};
each R^{AA} is independently selected from —OH, —NH₂, —CN or a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{AA} contains, in total, from 1 to 10 carbon, nitrogen and oxygen atoms;
each R^{B} is independently selected from a —CN, —NO₂, —R^{B1}, —OH, —OR^{B1}, —NH₂, —NHR^{B1} or —N(R^{B1})₂ group, wherein each R^{B1} is independently selected from a C₁-C₄ alkyl or C₁-C₄ fluoroalkyl group;
each Hal is independently selected from F, Cl or Br;
L² is a straight-chained alkylene or alkenylene group, wherein the straight-chained alkylene or alkenylene group optionally includes one or two heteroatoms independently selected from O and N in its carbon skeleton, wherein L² has a chain length of from 2 to 8 atoms, and wherein L² may optionally be substituted with one or two oxo (═O) groups and/or with one or more groups R^{L2}, wherein each R^{L2} is independently selected from a fluoro, C₁-C₄ alkyl, —O—(C₁-C₄ alkyl), C₁-C₄ fluoroalkyl or —O—(C₁-C₄ fluoroalkyl) group, or wherein any two R^{L2} may together with the atom(s) of the alkylene or alkenylene group to which they are attached form a 3- to 7-membered cyclic group, wherein the 3- to 7-membered cyclic group may optionally be substituted with one or more Hal groups and/or one or two oxo (═O) groups;
R⁴ is selected from a C₁-C₄ alkyl, C₁-C₄ fluoroalkyl, C₃-C₆ cycloalkyl or C₃-C₆ fluorocycloalkyl group, and R⁵ is selected from hydrogen, F, Cl, Br, or a —CN, methyl, fluoromethyl, —OC(R²⁰)₃ or —C(R²⁰)₂—OC(R²⁰)₃ group, or R⁴ and R⁵ together form a divalent group selected from —CH₂CH₂CH₂—, —CH═CHCH₂—, —CH₂CH═CH—, —CH₂CH₂O— and —OCH₂CH₂—, wherein the divalent group formed by R⁴ and R⁵ may optionally be fluoro-substituted;
R⁶ and R⁷ are each independently selected from hydrogen, F, Cl, Br, or a —CN, methyl, fluoromethyl, —OC(R²⁰)₃ or —C(R²⁰)₂—OC(R²⁰)₃ group; and
each R²⁰ is independently selected from hydrogen or F.

21. A compound or a pharmaceutically acceptable salt or solvate thereof, as claimed in claim 1, wherein the compound has the formula (If): 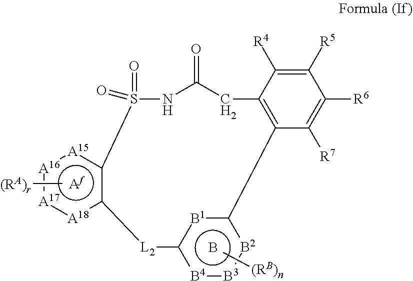
wherein:
A¹⁵, A¹⁶, A¹⁷ and A¹⁸ are each independently selected from N, C—H and C-Hal, such that ring A^{f} is a 6-membered aryl ring or a 6-membered heteroaryl ring containing one, two or three nitrogen atoms in its ring structure;
B¹, B², B³ and B⁴ are each independently selected from N, C—H and C-Hal, such that ring B is a 6-membered aryl ring or a 6-membered heteroaryl ring containing one, two or three nitrogen atoms in its ring structure;
r is 0, 1 or 2;
n is 0, 1 or 2;
each R^{A} is independently selected from —OH, —NH₂, —CN or a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{A} contains, in total, from 1 to 10 carbon, nitrogen and oxygen atoms, or wherein any two R^{A} attached to A¹⁵ and A¹⁶ or to A¹⁶ and A¹⁷ or to A¹⁷ and A18 may together form a fused 5- or 6-membered cyclic group, wherein the fused 5- or 6-membered cyclic group may optionally be substituted with one or more Hal groups and/or one or two groups independently selected from oxo (═O) and R^{AA};
each R^{AA} is independently selected from —OH, —NH₂, —CN or a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{AA} contains, in total, from 1 to 10 carbon, nitrogen and oxygen atoms;
each R^{B} is independently selected from a —CN, —NO₂, —R^{B1}, —OH, —OR^{B1}, —NH₂, —NHR^{B1} or —N(R^{B1})₂ group, wherein each R^{B1} is independently selected from a C₁-C₄ alkyl or C₁-C₄ fluoroalkyl group;
each Hal is independently selected from F, Cl or Br;
L² is a straight-chained alkylene or alkenylene group, wherein the straight-chained alkylene or alkenylene group optionally includes one or two heteroatoms independently selected from O and N in its carbon skeleton, wherein L² has a chain length of from 2 to 8 atoms, and wherein L² may optionally be substituted with one or two oxo (═O) groups and/or with one or more groups R^{L2}, wherein each R^{L2} is independently selected from a fluoro, C₁-C₄ alkyl, —O—(C₁-C₄ alkyl), C₁-C₄ fluoroalkyl or —O—(C₁-C₄ fluoroalkyl) group, or wherein any two R^{L2} may together with the atom(s) of the alkylene or alkenylene group to which they are attached form a 3- to 7-membered cyclic group, wherein the 3- to 7-membered cyclic group may optionally be substituted with one or more Hal groups and/or one or two oxo (═O) groups;
R⁴ is selected from a C₁-C₄ alkyl, C₁-C₄ fluoroalkyl, C₃-C₆ cycloalkyl or C₃-C₆ fluorocycloalkyl group, and R⁵ is selected from hydrogen, F, Cl, Br, or a —CN, methyl, fluoromethyl, —OC(R²⁰)₃ or —C(R²⁰)₂—OC(R²⁰)₃ group, or R⁴ and R⁵ together form a divalent group selected from —CH₂CH₂CH₂—, —CH═CHCH₂—, —CH₂CH═CH—, —CH₂CH₂O— and —OCH₂CH₂—, wherein the divalent group formed by R⁴ and R⁵ may optionally be fluoro-substituted;
R⁶ and R⁷ are each independently selected from hydrogen, F, Cl, Br, or a —CN, methyl, fluoromethyl, —OC(R²⁰)₃ or —C(R²⁰)₂—OC(R²⁰)₃ group; and
each R²⁰ is independently selected from hydrogen or F.

22. A compound or a pharmaceutically acceptable salt or solvate thereof, as claimed in claim 1, wherein the compound has the formula (Ig): 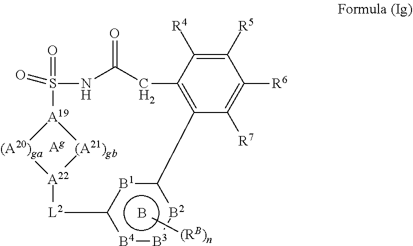
wherein:
A¹⁹ and A²² are each independently selected from N, CH, CY and CR^{AG}, and each A²⁰ and A²¹ is independently selected from O, NH, NR^{AGG}, C═O, CH₂, CH(Y), CH(R^{AG}), C(Y)₂, C(Y)(R^{AG}) and C(R^{AG})₂, such that ring Ag contains one or two atoms independently selected from oxygen and nitrogen in its ring structure;
ga is 1, 2 or 3, and gb is 1, 2 or 3, provided that ga+gb≤5;
each Y is independently selected from F, Cl or Br;
each R^{AG} is independently selected from —OH, —NH₂, —CN, or a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{AG} contains, in total, from 1 to 10 carbon, nitrogen and oxygen atoms;
each R^{AGG} is independently selected from a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{AGG} contains, in total, from 1 to 10 carbon, nitrogen and oxygen atoms;
B¹, B², B³ and B⁴ are each independently selected from N, C—H and C-Hal, such that ring B is a 6-membered aryl ring or a 6-membered heteroaryl ring containing one, two or three nitrogen atoms in its ring structure;
n is 0, 1 or 2;
each R^{B} is independently selected from a —CN, —NO₂, —R^{B1}, —OH, —OR^{B1}, —NH₂, —NHR^{B1} or —N(R^{B1})₂ group, wherein each R^{B1} is independently selected from a C₁-C₄ alkyl or C₁-C₄ fluoroalkyl group;
each Hal is independently selected from F, Cl or Br;
L² is a straight-chained alkylene or alkenylene group, wherein the straight-chained alkylene or alkenylene group optionally includes one or two heteroatoms independently selected from O and N in its carbon skeleton, wherein L² has a chain length of from 2 to 8 atoms, and wherein L² may optionally be substituted with one or two oxo (═O) groups and/or with one or more groups R^{L2}, wherein each R^{L2} is independently selected from a fluoro, C₁-C₄ alkyl, —O—(C₁-C₄ alkyl), C₁-C₄ fluoroalkyl or —O—(C₁-C₄ fluoroalkyl) group, or wherein any two R^{L2} may together with the atom(s) of the alkylene or alkenylene group to which they are attached form a 3- to 7-membered cyclic group, wherein the 3- to 7-membered cyclic group may optionally be substituted with one or more Hal groups and/or one or two oxo (═O) groups;
R⁴ is selected from a C₁-C₄ alkyl, C₁-C₄ fluoroalkyl, C₃-C₆ cycloalkyl or C₃-C₆ fluorocycloalkyl group, and R⁵ is selected from hydrogen, F, Cl, Br, or a —CN, methyl, fluoromethyl, —OC(R²⁰)₃ or —C(R²⁰)₂—OC(R²⁰)₃ group, or R⁴ and R⁵ together form a divalent group selected from —CH₂CH₂CH₂—, —CH═CHCH₂—, —CH₂CH═CH—, —CH₂CH₂O— and —OCH₂CH₂—, wherein the divalent group formed by R⁴ and R⁵ may optionally be fluoro-substituted;
R⁶ and R⁷ are each independently selected from hydrogen, F, Cl, Br, or a —CN, methyl, fluoromethyl, —OC(R²⁰)₃ or —C(R²⁰)₂—OC(R²⁰)₃ group; and
each R²⁰ is independently selected from hydrogen or F.

23. A compound or a pharmaceutically acceptable salt or solvate thereof, as claimed in claim 1, which is (a) a compound selected from the group consisting of: 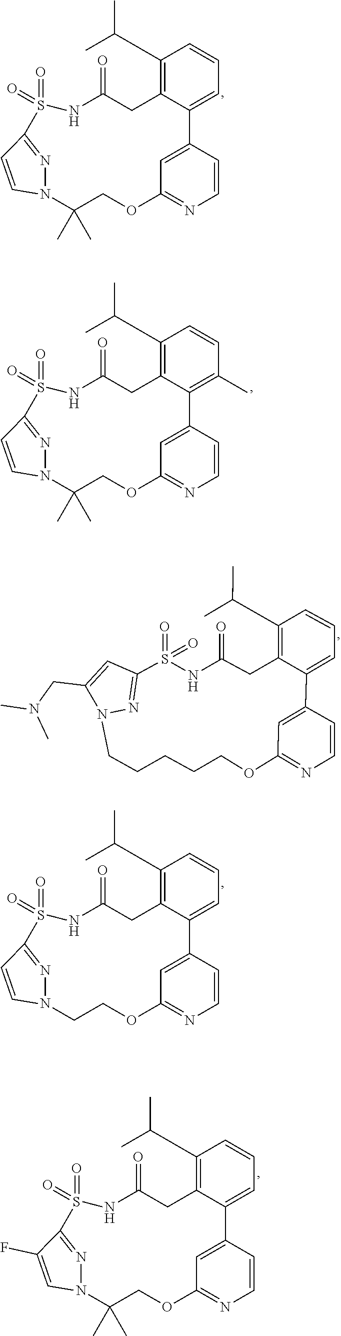 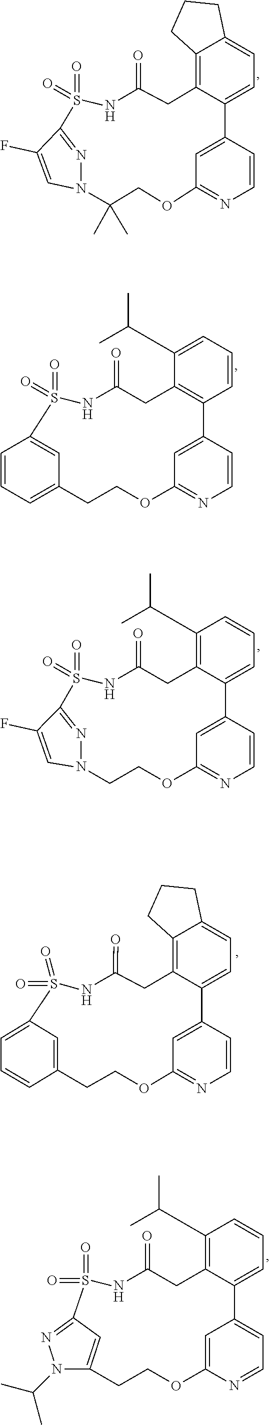 [SMILES: CC1(C)COc2cc(ccn2)-c2ccc3c(c2CC(=O)NS(=O)(=O)c2nn1cc2F)CCC3 ; CC(C)c1cccc2c1CC(=O)NS(=O)(=O)c1cccc(c1)CCOc1cc-2ccn1 ; CC(C)c1cccc2c1CC(=O)NS(=O)(=O)c1nn(cc1F)CCOc1cc-2ccn1 ; O=C1Cc2c(ccc3c2CCC3)-c2ccnc(c2)OCCc2cccc(c2)S(=O)(=O)N1 ; CC(C)c1cccc2c1CC(=O)NS(=O)(=O)c1cc(n(C(C)C)n1)CCOc1cc-2ccn1] 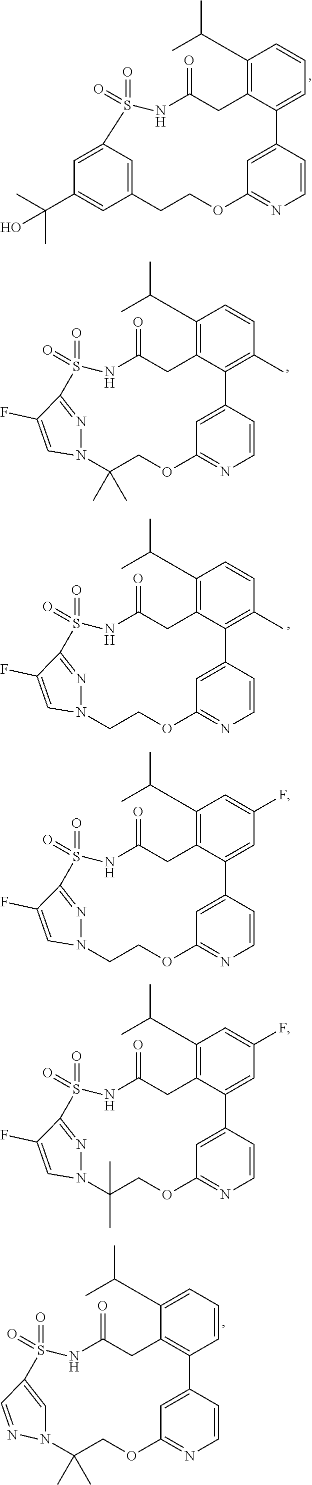 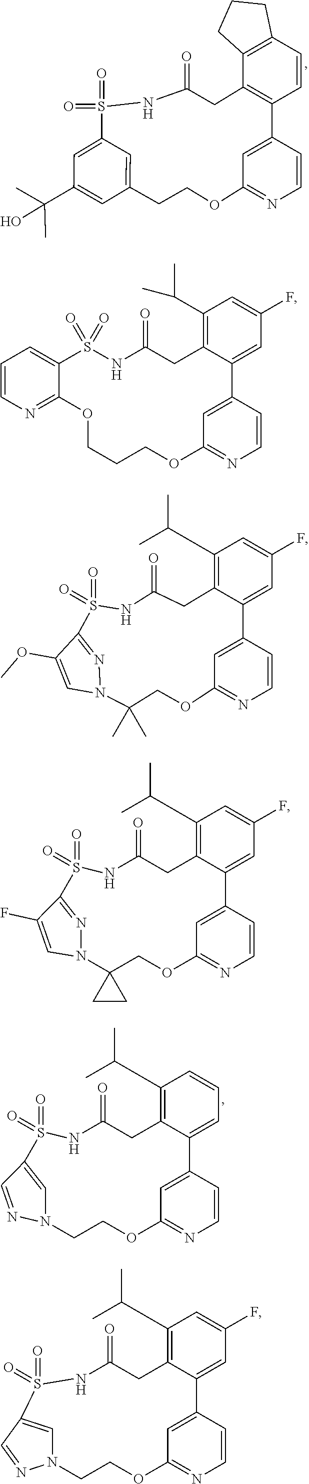 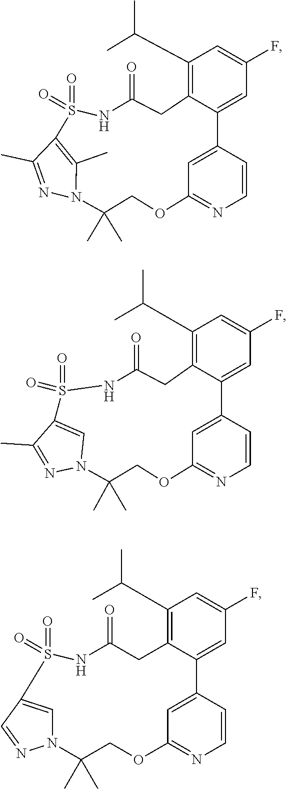 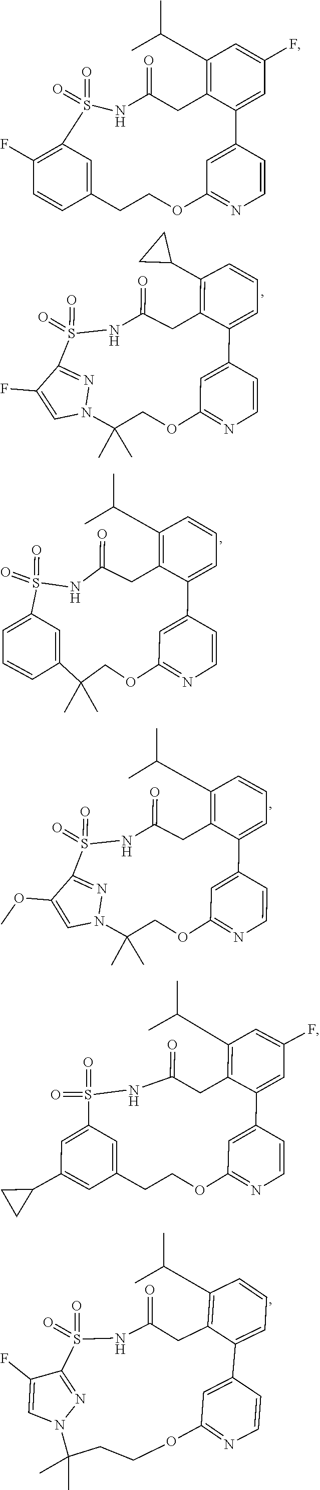 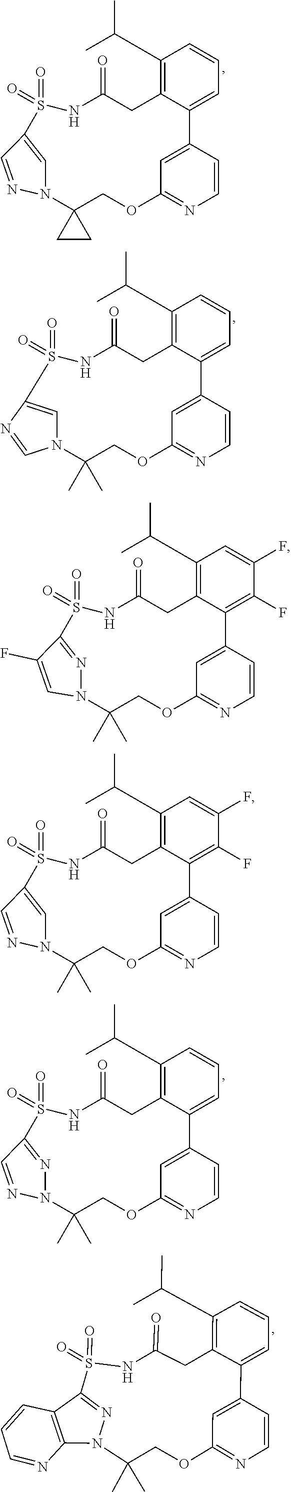 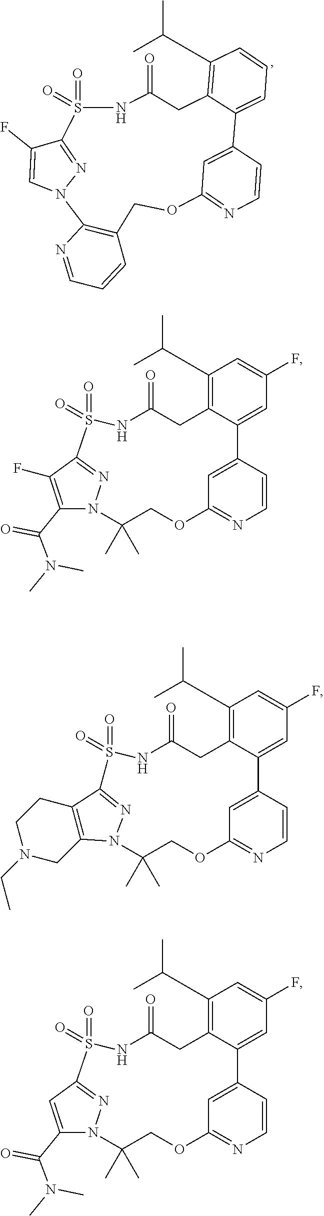 [SMILES: CC(C)c1cccc2c1CC(=O)NS(=O)(=O)c1nn(cc1F)-c1ncccc1COc1cc-2ccn1 ; CC(C)c1cc(F)cc2c1CC(=O)NS(=O)(=O)c1nn(c(C(=O)N(C)C)c1F)C(C)(C)COc1cc-2ccn1 ; CCN1CCc2c3nn(c2C1)C(C)(C)COc1cc(ccn1)-c1cc(F)cc(C(C)C)c1CC(=O)NS3(=O)=O ; CC(C)c1cc(F)cc2c1CC(=O)NS(=O)(=O)c1cc(C(=O)N(C)C)n(n1)C(C)(C)COc1cc-2ccn1] 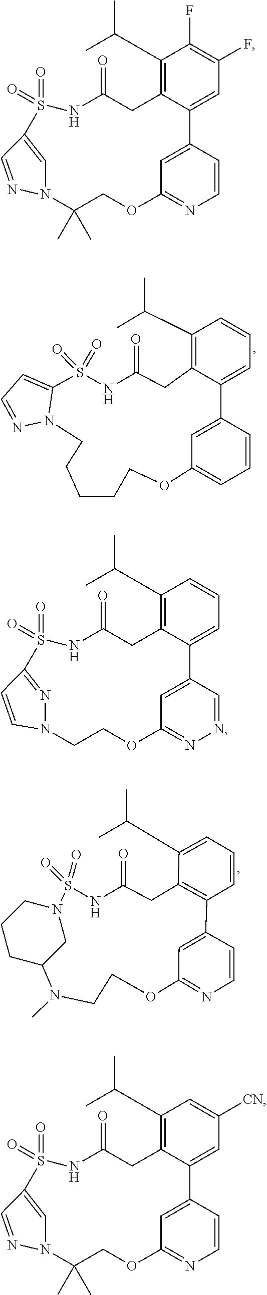 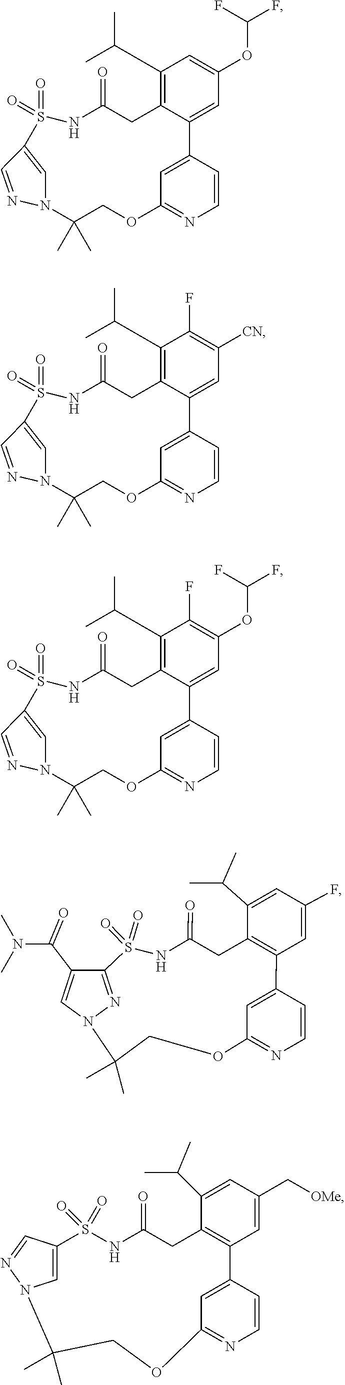 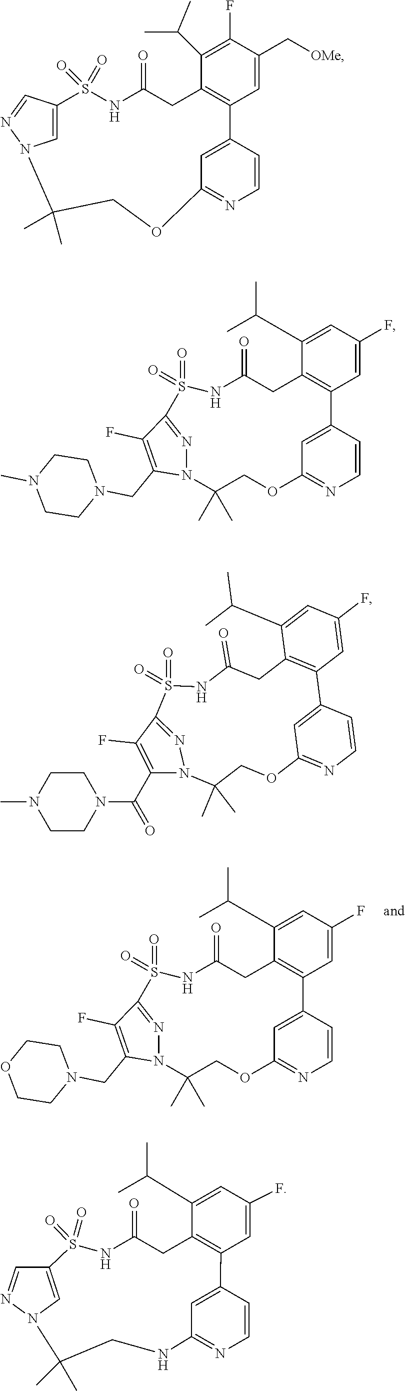
or (b) a pharmaceutically acceptable salt or solvate of the selected compound.

24. A prodrug of a compound as claimed in claim 1, or a pharmaceutically acceptable salt or solvate thereof.

25. A pharmaceutical composition comprising a compound or a pharmaceutically acceptable salt or solvate thereof, as claimed in claim 1, and a pharmaceutically acceptable excipient.

26. A method of treating or preventing a disease, disorder or condition in a subject, the method comprising the step of administering an effective amount of a compound or a pharmaceutically acceptable salt or solvate thereof, as claimed in claim 1, to the subject, thereby treating or preventing the disease, disorder or condition, optionally wherein the disease, disorder or condition is responsive to NLRP3 inhibition.

27. (canceled)

28. The method as claimed in claim 26 wherein the disease, disorder or condition is selected from:
(i) inflammation;
(ii) an auto-immune disease;
(iii) cancer;
(iv) an infection;
(v) a central nervous system disease;
(vi) a metabolic disease;
(vii) a cardiovascular disease;
(viii) a respiratory disease;
(ix) a liver disease;
(x) a renal disease;
(xi) an ocular disease;
(xii) a skin disease;
(xiii) a lymphatic condition;
(xiv) a psychological disorder;
(xv) graft versus host disease;
(xvi) pain;
(xvii) a condition associated with diabetes;
(xviii) a condition associated with arthritis;
(xix) a headache;
(xx) a wound or burn; and
(xxi) any disease where an individual has been determined to carry a germline or somatic non-silent mutation in NLRP3.

29. The method as claimed in claim 26, wherein the disease, disorder or condition is selected from:
(i) cryopyrin-associated periodic syndromes (CAPS);
(ii) Muckle-Wells syndrome (MWS);
(iii) familial cold autoinflammatory syndrome (FCAS);
(iv) neonatal onset multisystem inflammatory disease (NOMID);
(v) familial Mediterranean fever (FMF);
(vi) pyogenic arthritis, pyoderma gangrenosum and acne syndrome (PAPA);
(vii) hyperimmunoglobulinemia D and periodic fever syndrome (HIDS);
(viii) Tumour Necrosis Factor (TNF) Receptor-Associated Periodic Syndrome (TRAPS);
(ix) systemic juvenile idiopathic arthritis;
(x) adult-onset Still's disease (AOSD);
(xi) relapsing polychondritis;
(xii) Schnitzler's syndrome;
(xiii) Sweet's syndrome;
(xiv) Behcet's disease;
(xv) anti-synthetase syndrome;
(xvi) deficiency of interleukin 1 receptor antagonist (DIRA); and
(xvii) haploinsufficiency of A20 (HA20).

30. A method of inhibiting NLRP3 in a subject, the method comprising administering a compound or a pharmaceutically acceptable salt or solvate thereof as claimed in claim 1, to the subject thereby inhibiting NLRP3.

31. A method of analysing inhibition of NLRP3 or an effect of inhibition of NLRP3 by a compound, comprising contacting a cell or non-human animal with a compound or a pharmaceutically acceptable salt or solvate thereof, as claimed in claim 1, and analysing inhibition of NLRP3 or an effect of inhibition of NLRP3 in the cell or non-human animal by the compound.

32. The method as claimed in claim 26, wherein the compound is administered as a pharmaceutical composition further comprising a pharmaceutically acceptable excipient.

33. A pharmaceutical composition comprising a prodrug or a pharmaceutically acceptable salt or solvate thereof, as claimed in claim 24, and a pharmaceutically acceptable excipient.
